(19) United States
(12) Patent Application Publication
(10) Pub. No.: US 2024/0092739 A1
(43) Pub. Date: Mar. 21, 2024

(54) COMPOUNDS AND METHODS OF USE

(71) Applicant: Ferro Therapeutics, Inc, Palo Alto, CA (US)

(72) Inventors: Chun Jiang, Hillsborough, CA (US); Bin Wang, Dallas, TX (US); Rui Xu, Dallas, TX (US); Eli Wallace, Loveland, CO (US); Chunhe Xie, Palo Alto, CA (US)

(21) Appl. No.: 18/023,270

(22) PCT Filed: Aug. 26, 2021
(86) PCT No.: PCT/CN2021/114827

§ 371 Date: Feb. 24, 2023

Related U.S. Application Data
(60) Provisional application No. 63/070,750, filed on Aug. 26, 2020.
(60) Provisional application No. 63/070,757, filed on Aug. 26, 2020.

(51) Int. Cl. C07D 217/14; A61P 35/00; C07D 401/10; C07D 401/12; C07D 471/04; C07D 495/04; C07F 7/08

(52) U.S. Cl. CPC C07D 217/14; A61P 35/00; C07D 401/10; C07D 401/12; C07D 471/04; C07D 495/04; C07F 7/081

(57) ABSTRACT

Disclosed are heterocyclic compounds or their pharmaceutically acceptable salts with ferroptosis-inducing activity, Those compounds or their pharmaceutically acceptable salts can be used to treat cancer.

### CROSS REFERENCE TO RELATED APPLICATIONS

This application claims the benefit under 35 U.S.C. § 119(e) to U.S. Provisional application Nos. 63/070,750, filed Aug. 26, 2020 and 63/070,757, filed Aug. 26, 2020, each of which is incorporated by reference in its entirety.

### BACKGROUND

Glutathione peroxidase 4 (GPX4) can directly reduce phospholipid hydroperoxide. Depletion of GPX4 induces lipid peroxidation-dependent cell death. Cancer cells in a drug-induced, therapy-resistant state have an enhanced dependence on the lipid peroxidase activity of GPX4 to prevent undergoing ferroptotic cell death. Studies have shown that lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis (see, e.g., Viswanathan et al., Nature 547:453-7, 2017). It has also been experimentally determined that that GPX4 inhibition (GPX4i) can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, a GPX4 inhibitor can be useful to induce ferroptotic cancer cell death and thus treat cancer.

### SUMMARY

The present disclosure relates to compounds having ferroptosis inducing activity, and methods of using the compounds for, e.g., the treatment of cancer. Provided herein are compounds, such as a compound of Formula A-I, A-IA, A-IB, A-II, A-III, B-I, B-IA, B-IB, B-II, B-IIA, B-IIB, B-X, B-XIA, or B-XIB, or a tautomer, stereoisomer, mixture of stereoisomers, isotopically enriched analog, or pharmaceutically acceptable salt thereof, as well as compounds of Tables A-IA, A-IB, A-IC, A-2, A-3, B-1, B-2, B-3, B-4, B-5, B-6, B-7, B-XIA, and B-XIB, or a tautomer, stereoisomer, mixture of stereoisomers, isotopically enriched analog, or pharmaceutically acceptable salt thereof, as well as their use in compositions and methods disclosed herein.

In an aspect, the present disclosure provides a compound of Formula A-I:

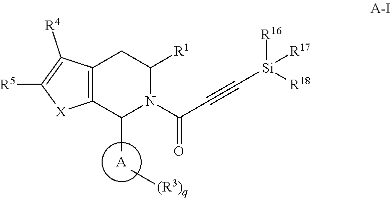

or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R⁵, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)R¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, at least one of R¹⁶, R¹⁷, and R¹⁸ is other than unsubstituted methyl.

In certain embodiments, the compound is not:

Structure

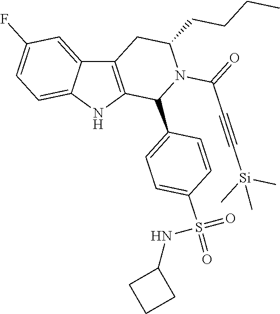

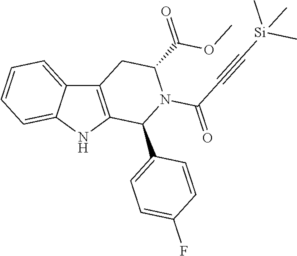 [SMILES: COC(=O)[C@H]1Cc2c([nH]c3ccccc23)[C@H](c2ccc(F)cc2)N1C(=O)C#C[Si](C)(C)C]

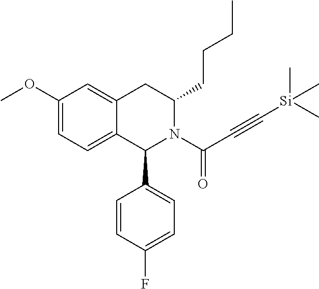

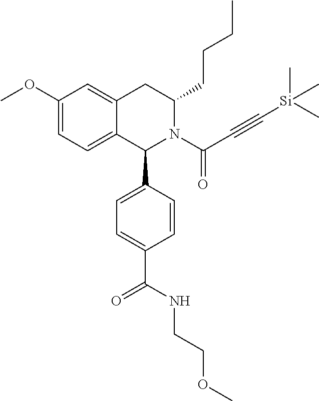

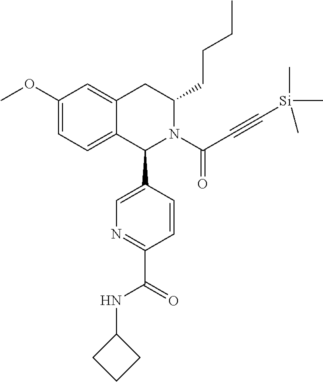

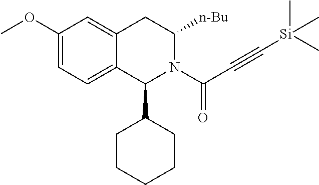

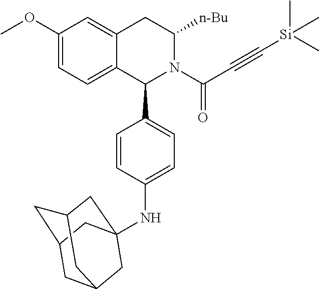

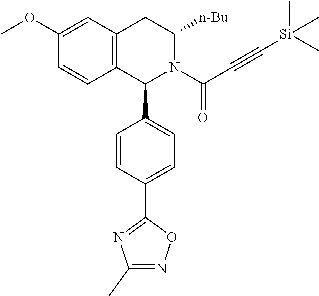

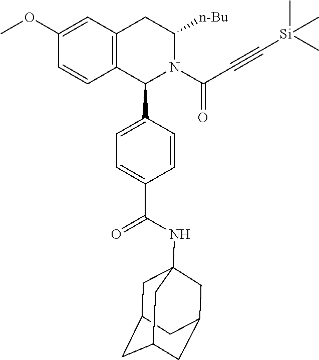

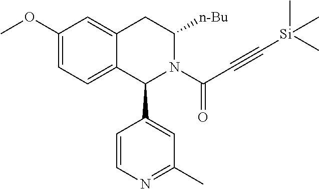

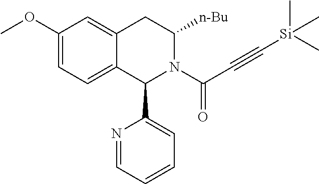

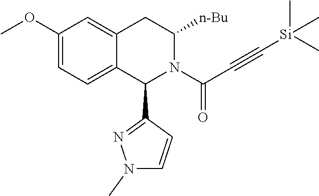

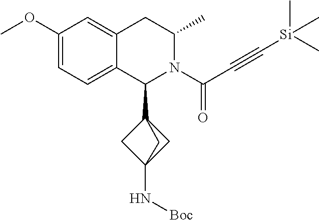

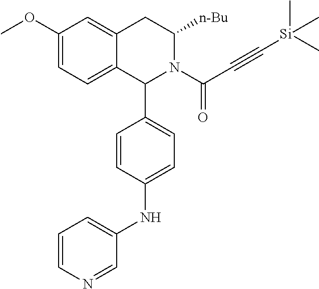

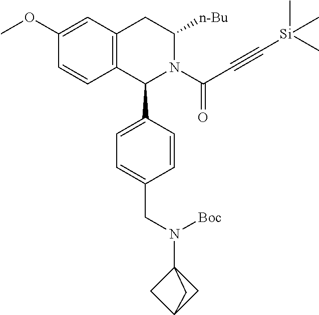

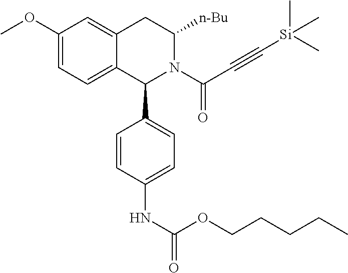

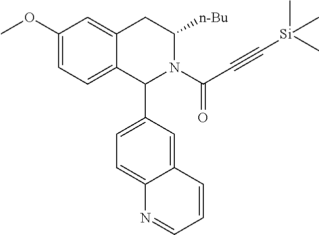

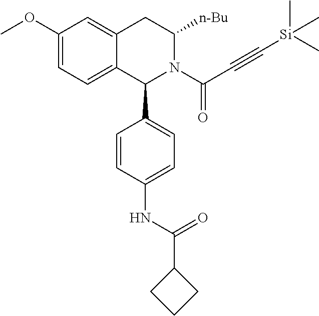

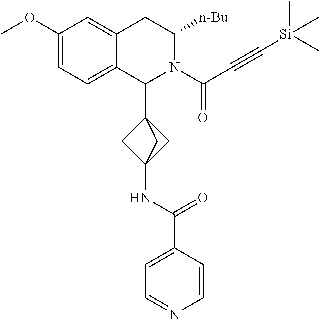

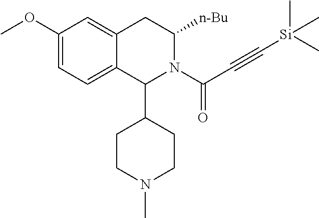

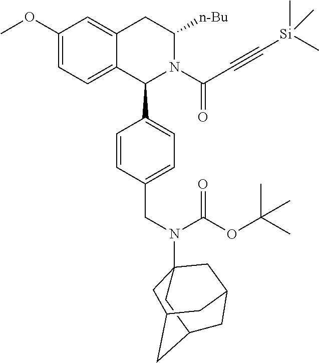

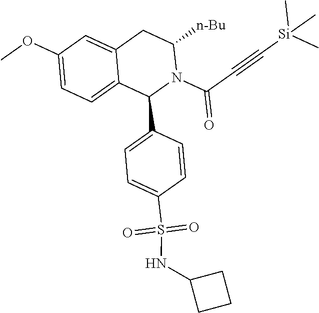

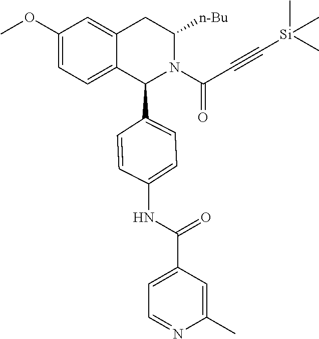

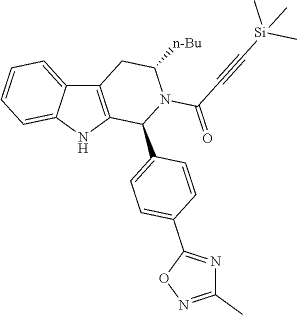

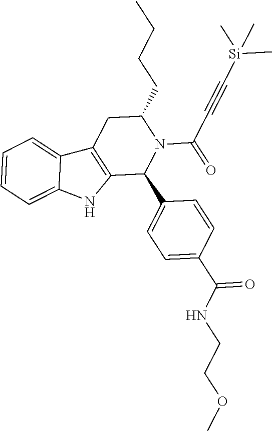 [SMILES: CCCC[C@H]1Cc2c([nH]c3ccccc23)[C@H](c2ccc(C(=O)NCCOC)cc2)N1C(=O)C#C[Si](C)(C)C]

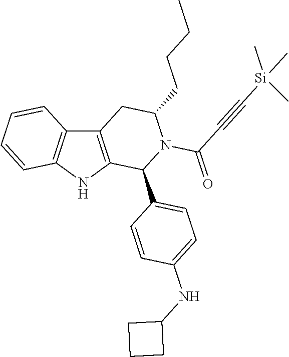 [SMILES: CCCC[C@H]1Cc2c([nH]c3ccccc23)[C@H](c2ccc(NC3CCC3)cc2)N1C(=O)C#C[Si](C)(C)C]

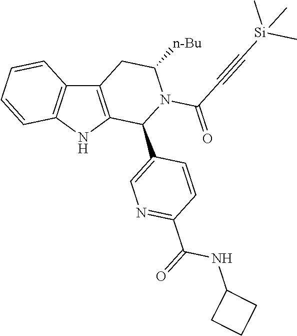

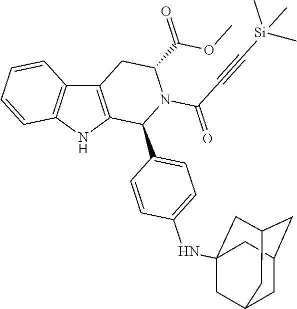 [SMILES: COC(=O)[C@H]1Cc2c([nH]c3ccccc23)[C@H](c2ccc(NC34CC5CC(CC(C5)C3)C4)cc2)N1C(=O)C#C[Si](C)(C)C]

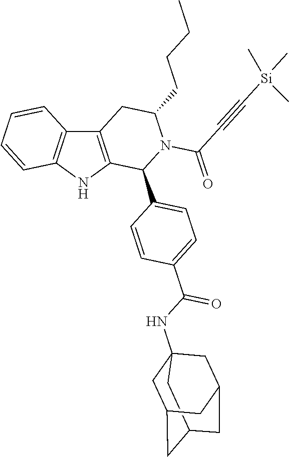 [SMILES: CCCC[C@H]1Cc2c([nH]c3ccccc23)[C@H](c2ccc(C(=O)NC34CC5CC(CC(C5)C3)C4)cc2)N1C(=O)C#C[Si](C)(C)C]

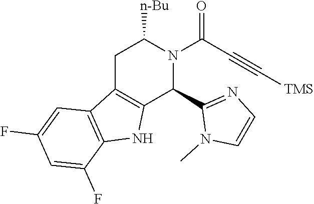

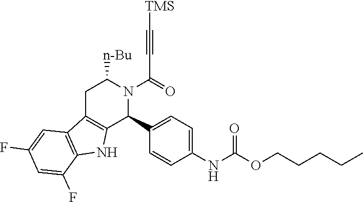

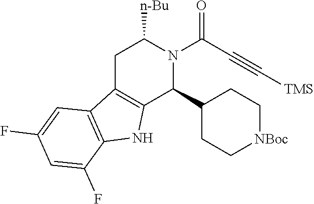

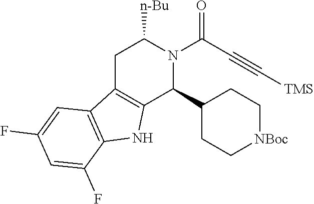

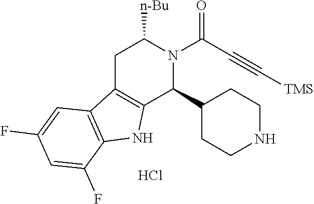

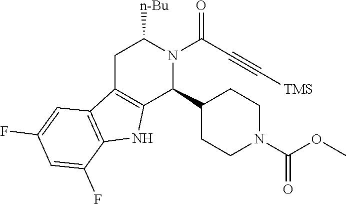

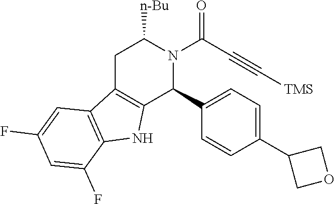

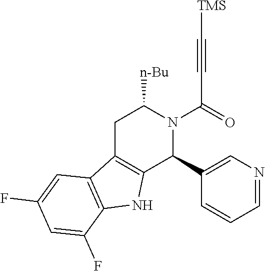

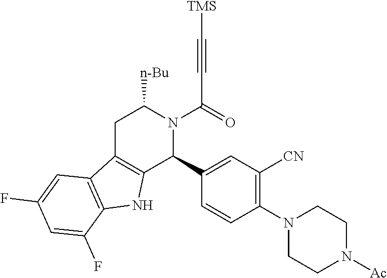

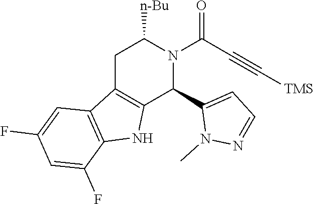

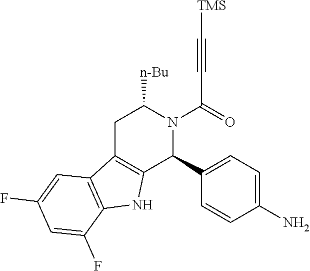

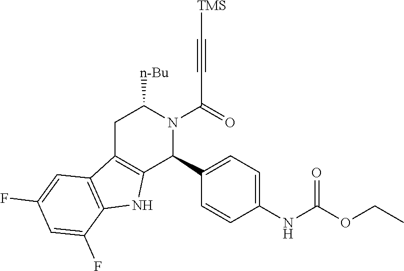

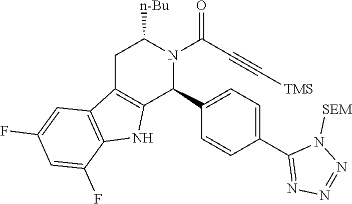

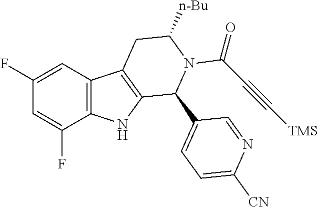

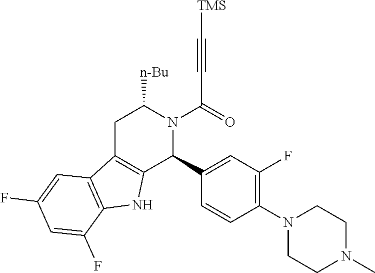

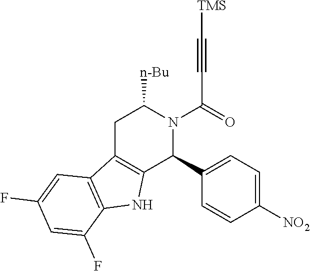

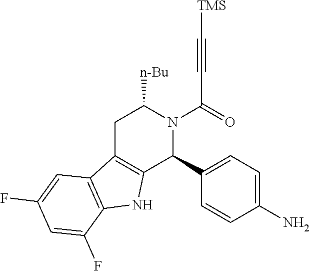

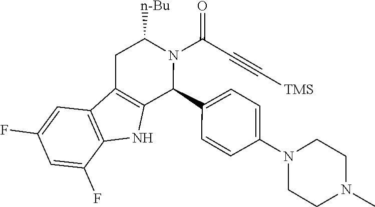

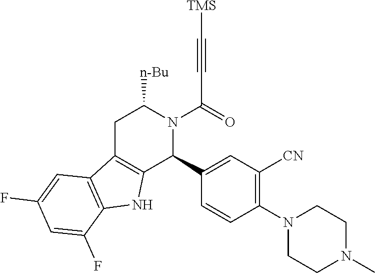

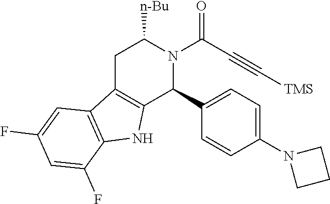

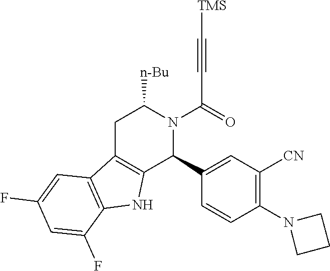

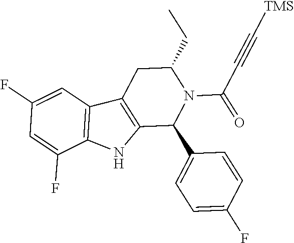 [SMILES: CC[C@H]1Cc2c([nH]c3c(F)cc(F)cc23)[C@H](c2ccc(F)cc2)N1C(=O)C#C[Si](C)(C)C]

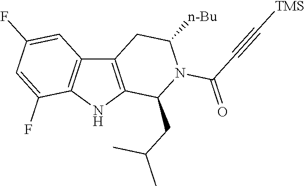

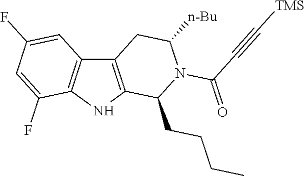

In certain embodiments, the compounds exhibit GPX4 inhibiting activity, and in certain embodiments, exhibit altered or enhanced stability (e.g., metabolic stability) and/or enhanced activity or other characteristics as compared to other GPX4 inhibitors. In certain embodiments, the compounds described herein are selective for GPX4 over other GPXs. In certain embodiments, the compounds are used in a method of inhibiting GPX4 in a cell, comprising contacting a cell with an effective amount of the compound described herein to inhibit GPX4 in the cell. In certain embodiments, the cell is a cancer cell.

In another aspect, the present disclosure provides a method of inducing ferroptosis in a cell comprising contacting the cell with an effective amount of a compound or composition provided herein, or a compound of formula A-I:

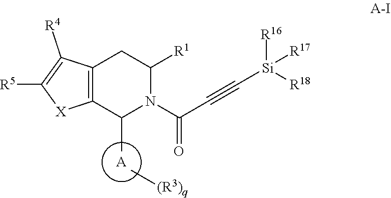

or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R is independently unsubstituted or substituted with one to three R¹⁰; or
when X is —NR²²—, —O—, or —S—, then R⁴ and R, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In a further aspect, the present disclosure provides a method for treating a disease or disorder (e.g., a cancer) in a subject in need thereof, comprising administering an effective amount of a compound or composition provided herein, or a compound of formula A-I:

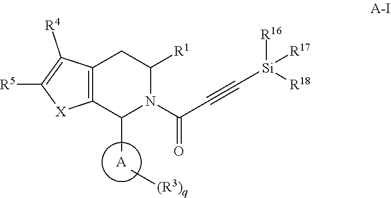

or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R⁵, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)R¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In another aspect, the present disclosure provides a method for treating a malignant solid tumor in a subject in need thereof, comprising administering to the subject an effective amount of a compound or composition provided herein, or a compound of formula A-I:

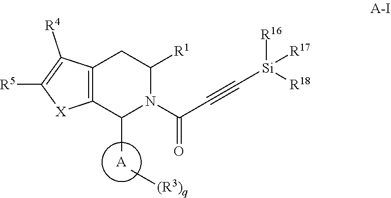

or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R⁵, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰. In certain embodiments, the malignant solid tumor is a sarcoma, carcinoma, or lymphoma.

### DETAILED DESCRIPTION

As used in this specification and the appended claims, the singular forms “a,” “an,” and “the” include plural referents unless the context clearly indicates otherwise. Thus, for example, reference to “a protein” includes more than one protein, and reference to “a compound” refers to more than one compound.

Also, the use of “or” means “and/or” unless stated otherwise. Similarly, “comprise,” “comprises,” “comprising” “include,” “includes,” and “including” are interchangeable and not intended to be limiting.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

It is to be further understood that where descriptions of various embodiments use the term “comprising,” those skilled in the art would understand that in some specific instances, an embodiment can be alternatively described using language “consisting essentially of” or “consisting of.”

It is to be understood that both the foregoing general description, including the drawings, and the following detailed description are exemplary and explanatory only and are not restrictive of this disclosure. The section headings used herein are for organizational purposes only and not to be construed as limiting the subject matter described.

### 1. Definitions

In reference to the present disclosure, the technical and scientific terms used in the descriptions herein will have the meanings commonly understood by one of ordinary skill in the art, unless specifically defined otherwise. Accordingly, the following terms are intended to have the meanings as described below.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Ferroptosis” refers to a form of cell death understood in the art as involving generation of reactive oxygen species mediated by iron, and characterized by, in part, lipid peroxidation.

“Ferroptosis inducer” or “ferroptosis activator” refers to an agent which induces, promotes, or activates ferroptosis.

“GPX4 inhibitor” refers to any agent that inhibits, or in certain embodiments at least partially inhibits, the activity of the enzyme glutathione peroxidase 4 (GPX4). A GPX4 inhibitor can be either a direct or indirect inhibitor. GPX4 is a phospholipid hydroperoxidase that catalyzes the reduction of hydrogen peroxide and organic peroxides, thereby protecting cells against membrane lipid peroxidation, or oxidative stress. Without wishing to be bound by theory, it is believed that GPX4 has a selenocysteine in the active site that is oxidized to a selenenic acid by a peroxide to afford a lipid-alcohol. The glutathione acts to reduce the selenenic acid (—SeOH) back to the selenol (—SeH). Should this catalytic cycle be disrupted, cell death occurs through an intracellular iron-mediated process known as ferroptosis. In certain embodiments a GPX4 inhibitor may be a compound that exhibits an IC₅₀ with respect to GPX4 activity of less than about 10 micromolar (μM), such as less than about 5 μM, 1 μM, 500 nM, 200 nM, 100 nM, 50 nM, or less.

“Subject” as used herein refers to a mammal. Non-limiting examples of mammals include humans, rodents (e.g., rates, mice, squirrels, guinea pigs, hamsters, etc.), lagomorphs (e.g., rabbits, hares, etc.), cats, dogs, non-human primates (e.g., monkeys, apes, etc.), goats, pigs, sheep, cows, deer, horses, and marsupials, for example in certain embodiments, is a dog, a cat, a horse, or a rabbit. In certain embodiments, the subject is a non-human primate, for example a monkey, chimpanzee, or gorilla. In certain embodiments, the subject is a human, sometimes referred to herein as a patient.

“Treating” or “treatment” of a disease, disorder, or syndrome, as used herein, includes (i) preventing the disease, disorder, or syndrome from occurring in a subject, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art, particularly in view of the guidance provided in the present disclosure.

In certain embodiments, “treat,” “treating,” or “treatment” refer to any indicia of success in the treatment or amelioration of a condition, disease, disorder, or syndrome, including any objective or subjective parameter such as abatement; remission, diminishing of symptoms or making the condition, disease, disorder, or syndrome, or symptom thereof, more tolerable to a subject; slowing in the rate of degeneration or decline; making the final point of degeneration less debilitating; and/or improving a subject's physical or mental well-being. The treatment or amelioration of symptoms can be based on objective or subject parameters, including the results of a physical examination, neuropsychiatric examination, and/or a psychiatric evaluation. Treatment may also be preemptive in nature; e.g., it may include prevention of a syndrome, disease, disorder or condition; prevention of onset of one or more symptoms of a syndrome, disease, disorder, or condition; and/or prevention of escalation of a syndrome, disease, disorder, or condition. Prevention of a syndrome, disease, disorder, or condition may involve complete protection from disease, and/or prevention of disease progression (e.g., to a later stage of the syndrome, disease, disorder, or condition). For example, prevention of a disease may not mean complete foreclosure of any effect related to a disease at any level, but may instead mean prevention of one or more symptoms of a syndrome, disease, disorder, or condition to a clinically significant or detectable level. In some embodiments, treating comprises (i) preventing the condition, disease, disorder, or syndrome, or a symptom thereof, from occurring in a subject, i.e. causing one or more clinical symptoms of the condition, disease, disorder, or syndrome not to develop, or to develop less fully or more slowly, in a subject (e.g., a subject that may be exposed to or predisposed to the condition, disease, disorder, or syndrome but does not yet experience or display the one or more symptoms of the condition, disease, disorder, or syndrome); (ii) inhibiting the condition, disease, disorder, or syndrome, i.e., at least partially arresting its development or the development of one or more symptoms thereof; and/or (iii) relieving the condition, disease, disorder, or syndrome or a symptom thereof, i.e., causing at least partial regression of the condition, disease, disorder, or syndrome, or a symptom thereof. Adjustments for systemic versus localized delivery, age, body weight, general health, sex, diet, time of administration, drug interaction, and the severity of the condition may be necessary to achieve efficacy, and will be ascertainable by a skilled practitioner, particularly in view of the guidance provided in the present disclosure.

“Therapeutically effective amount” refers to that amount of a compound or of a pharmaceutical composition useful for treating or ameliorating an identified syndrome, disease, disorder, or condition, or for exhibiting a detectable therapeutic or inhibitory effect. A therapeutically effective amount can be an amount which, when administered to an animal (e.g., human) for treating a disease, disorder, condition, or syndrome, is sufficient to effect such treatment for the disease, disorder, condition, or syndrome. “Therapeutically effective amount” may refer to that amount which, when administered to an animal (e.g., human) for treating a disease, is sufficient to effect such treatment for the disease, disorder, or condition. In certain embodiments, the treatment provides a therapeutic benefit such as amelioration of symptoms or slowing of disease progression. For example, a therapeutically effective amount may be an amount sufficient to decrease a symptom of a disease or condition of as described herein. An exact amount will depend on the purpose and mechanism of the treatment, and will be ascertainable by a skilled practitioner.

“Alkyl,” as used herein, alone or in combination, refers to a straight or branched chain hydrocarbon group of 1 to 20 carbon atoms (C₁-C₂₀ or C₁₋₂₀ alkyl), e.g., 1 to 12 carbon atoms (C₁-C₁₂ or C₁₋₁₂ alkyl), or 1 to 8 carbon atoms (C₁-C₈ or C₁₋₈ alkyl). Examples of alkyl radicals include, but are not limited to, methyl, ethyl, n-propyl, isopropyl (e.g., i-propyl), n-butyl, isobutyl, sec-butyl (e.g., s-butyl), tert-butyl (e.g., t-butyl), n-pentyl, s-pentyl, iso-amyl, hexyl, octyl, noyl, and the like.

“Alkenyl,” as used herein, alone or in combination, refers to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂₋₈), having at least one double bond. Examples of alkenyl groups include, but are not limited to, vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, and the like.

“Alkynyl,” as used herein, alone or in combination, refers to a straight or branched chain hydrocarbon group of 2 to 12 carbon atoms (C₂-C₁₂ or C₂₋₁₂), e.g., 2 to 8 carbon atoms (C₂-C₈ or C₂₋₈), containing at least one triple bond. Examples of alkynyl groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl, and the like.

“Alkylene,” “alkenylene,” and “alkynylene,” as used herein, alone or in combination, refer to a straight or branched chain divalent hydrocarbon radical of the corresponding alkyl, alkenyl, and alkynyl, respectively. In certain embodiments, “alkyl,” “alkenyl,” and “alkynyl” can represent the corresponding “alkylene,” “alkenylene” and “alkynylene,” such as, by way of example and not limitation, cycloalkylalkyl-, heterocycloalkylalkyl-, arylalkyl-, heteroarylalkyl-, cycloalkylalkenyl-, heterocycloalkylalkenyl-, arylalkenyl-, heteroarylalkenyl-, cycloalkylalkynyl-, heterocycloalkylalkynyl-, arylalkynyl-, heteroarylalkynyl-, and the like, wherein the cycloalkyl, heterocycloalkyl, aryl, and heteroaryl group is connected, as a substituent via the corresponding alkylene, alkenylene, or alkynylene group.

“Lower” in reference to substituents refers to a group having between one and six carbon atoms.

“Alkylhalo” or “haloalkyl,” as used herein, alone or in combination, refer to a straight or branched chain hydrocarbon group of 1 to 20 carbon atoms (C₁-C₂₀ or C₁₋₂₀), e.g., 1 to 12 carbon atoms (C₁-C₁₂ or C₁₋₁₂), or 1 to 8 carbon atoms (C₁-C₈ or C₁₋₈) wherein one or more (e.g., one to three, or one) hydrogen atom is replaced by a halogen (e.g., Cl, F, etc.). In certain embodiments, the term “alkylhalo” refers to an alkyl group as defined herein, wherein one hydrogen atom is replaced by a halogen (e.g., Cl, F, etc.). In certain embodiments, an alkylhalo group includes multiple halogen atoms. For example, the alkylhalo group may be —CHF₂ or —CF₃. In certain embodiments, the term “alkylhalo” refers to an alkylflouoride or alkylchloride.

“Alkenylhalo” or “haloalkenyl,” as used herein, alone or in combination, refer to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂₋₈), having at least one double bond, wherein one or more (e.g., one to three, or one) hydrogen atom is replaced by a halogen (e.g., Cl, F, etc.). In certain embodiments, the term “alkenylhalo” refers to an alkenyl group as defined herein, wherein one hydrogen atom is replaced by a halogen (e.g., Cl, F, etc.). In certain embodiments, an alkyenylhalo group includes multiple halogen atoms. In certain embodiments, the term “alkenylhalo” refers to an alkenylfluoride or alkenylchloride.

“Cycloalkyl,” or, alternatively, “carbocycle,” as used herein, alone or in combination, refers to any stable monocyclic or polycyclic system which consists of carbon atoms, any ring of which being saturated (i.e., non-aromatic). A cycloalkyl group may be a monocyclic or multicyclic (e.g., bicyclic, tricyclic, etc.) group wherein each cyclic moiety contains from 3 to 12 carbon atom ring members. A cycloalkyl group may comprise fused rings, a bridged ring system, and/or a spiro ring system (e.g., a system including two rings sharing a single carbon atom). “Cycloalkenyl,” as used herein, alone or in combination, refers to any stable monocyclic or polycyclic system which consists of carbon atoms, with at least one ring thereof being partially unsaturated (e.g., having one or more double bonds). Examples of cycloalkyls include, but are not limited to, cyclopropyl, cyclobutyl, cyclopentyl, cyclohexyl, cycloheptyl, cyclobutenyl, cyclopentenyl, cyclohexenyl, tetrahydronaphthyl, indanyl, octahydronaphthyl, 2,3-dihydro-1H-indenyl, cyclooctyl, bicycloalkyls and tricycloalkyls (e.g., adamantyl). In some embodiments, a cycloalkyl group comprises from 5 to 7 carbon atoms. In some embodiments, a cycloalkyl group consists of from 5 to 7 carbon atoms. “Bicyclic” and “tricyclic” as used herein are intended into include both fused ring systems as well as multicyclic (multicentered) saturated or partially unsaturated ring systems.

“Heterocycloalkyl” or “heterocyclyl,” as used herein, alone or in combination, refer to a 4 to 14 membered, mono- or polycyclic (e.g., bicyclic), non-aromatic hydrocarbon ring, wherein 1 to 3 carbon atoms are replaced by a heteroatom. A heterocycle may be a saturated, partially unsaturated, or fully unsaturated monocyclic, bicyclic, or tricyclic heterocyclic group containing at least one heteroatom as a ring member, wherein each heteroatom may be independently selected from nitrogen, oxygen, sulfur, and phosphorus. Heteroatoms and/or heteroatomic groups which can replace the carbon atoms include, but are not limited to, —O—, —S—, —S—O—, —NR⁴⁰—, —PH—, —C(O)—, —S(O)—, —S(O)₂—, —S(O)NR⁴⁰—, —S(O)₂NR⁴⁰—, and the like, including combinations thereof, where each R⁴⁰ is independently hydrogen or lower alkyl. Examples include, but are not limited to, thiazolidinyl, thiadiazolyl, triazinyl, morpholinyl, pyrrolidinonyl, pyrrolidinyl, piperidinyl, piperazinyl, 2,3-dihydrofuranyl, dihydropyranyl, hydantoinyl, valerolactamyl, oxiranyl, oxetanyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and the like. In certain embodiments, the “heterocycloalkyl” or “heterocyclyl” is a substituted or unsubstituted 4 to 7 membered monocyclic ring, wherein 1 to 3 carbon atoms are replaced by a heteroatom as described above.

In certain embodiments, the “heterocycloalkyl” or “heterocyclyl” is a 4 to 10, or 4 to 9, or 5 to 9, or 5 to 7, or 5 to 6 membered mono- or polycyclic (e.g., bicyclic) ring, wherein 1 to 3 carbon atoms are replaced by a heteroatom as described above. In certain embodiments, when the “heterocycloalkyl” or “heterocyclyl” is a substituted or unsubstituted bicyclic ring, one ring may be aromatic, provided at least one ring is non-aromatic, regardless of the point of attachment to the remainder of the molecule (e.g., indolinyl, isoindolinyl, and the like).

“Aryl,” as used herein, alone or in combination, refers to a 6 to 14-membered, mono- or bi-carbocyclic ring, wherein the monocyclic ring is aromatic and at least one of the rings in the bicyclic ring is aromatic. An aryl group may be a 5 to 20-membered, such as a carbocyclic aromatic system containing one or more rings, where rings of a polycyclic ring system are fused together. Unless stated otherwise, the valency of the group may be located on any atom of any ring within the radical, valency rules permitting. Examples of “aryl” groups include, but are not limited to, phenyl, naphthyl, indenyl, biphenyl, phenanthrenyl, naphthacenyl, and the like. In some embodiments, an aryl group includes 5 or 6 carbon atoms.

“Heteroaryl,” as used herein, alone or in combination, means an aromatic heterocyclic ring, including monocyclic and polycyclic (e.g., bicyclic or tricyclic) ring systems, where at least one carbon atom of one or both of the rings is replaced with a heteroatom independently selected from nitrogen, oxygen, and sulfur, or at least two carbon atoms of one or both of the rings are replaced with a heteroatom independently selected from nitrogen, oxygen, and sulfur. In certain embodiments, the heteroaryl can be a 5 to 6 membered monocyclic, or 7 to 11 membered bicyclic ring systems. In some embodiments, a heteroaryl includes at least 1 heteroatom as a ring member, such as at least 1, 2, or 3 heteroatoms. In some embodiments, a heteroaryl includes between 1 and 3 heteroatoms. In a fused ring system, at least one of the fused rings may be aromatic and include at least one heteroatom. In some embodiments, a heteroaryl ring may be fused to a non-aromatic ring such as a carbocycle or heterocycle. In some embodiments, heteroaryl ring may be fused to another heteroaryl ring. Examples of “heteroaryl” groups include pyrrolyl, pyrrolinyl, pyrimidinyl, pyridazinyl, pyranyl, oxadiazolyl, thiadiazolyl, isothiazoyl, isoindolyl, indolizinyl, pyrazolyl, imidazolyl, pyrazinyl, oxazolyl, isoxazolyl, thiazolyl, furyl, thienyl, pyridyl, pyrimidyl, benzothiazolyl, purinyl, benzimidazolyl, indolyl, isoquinolyl, quinoxalinyl, quinazolinyl, quinolyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl, carbazolyl, benzindolyl, phenanthrolinyl, dibenzofuranyl, acridinyl, phenanthridinyl, xanthenyl, and the like.

“Bridged bicyclic,” as used herein, refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 5-12 ring members and 0-4 heteroatoms independently selected from nitrogen, oxygen, and sulfur. Such bridged bicyclic groups include those groups set forth below where each group is attached to the rest of the molecule at any substitutable carbon or nitrogen atom.

Exemplary bridged bicyclics include, but are not limited to:

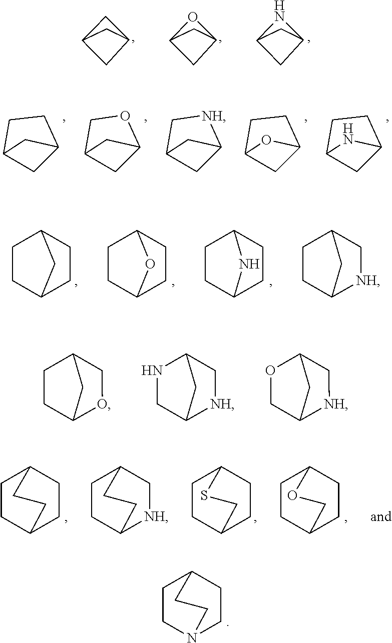

“Fused ring,” as used herein, refers a ring system with two or more rings having at least one bond and two atoms in common. A “fused aryl” and a “fused heteroaryl” refer to ring systems having at least one aryl and heteroaryl, respectively, that share at least one bond and two atoms in common with another ring.

“Halogen” or “halo” refers to fluorine, chlorine, bromine, and iodine.

“Acyl” refers to —C(O)R⁴³, where R⁴³ is hydrogen, or a group selected from alkyl, heteroalkyl, cycloalkyl, cycloalkylalkyl, heterocycloalkyl, heterocycloalkylalkyl, aryl, arylalkyl, heteroaryl, or heteroarylalkyl as defined herein. Examples of acyl groups include, but are not limited to, formyl, acetyl, cyclohexylcarbonyl, cyclohexylmethylcarbonyl, benzoyl, benzylcarbonyl, and the like.

“Alkyloxy” or “alkoxy” refers to —OR⁴⁴, wherein R⁴⁴ is an alkyl group that is unsubstituted or is substituted with one or more substituents.

“Aryloxy” refers to —OR⁴⁵, wherein R⁴⁵ is an aryl that is unsubstituted or is substituted with one or more substituents.

“Carboxy” refers to —COO⁻ or COOM, wherein M is hydrogen or a counterion (e.g., a cation, such as Na⁺, Ca²⁺, Mg²⁺, etc.).

“Carbamoyl” refers to —C(O)NR⁴⁶R⁴⁶, wherein each R⁴⁶ is independently selected from H or an a group selected from alkyl, cycloalkyl, cycloalkylalkyl, heterocycloalkyl, heterocycloalkylalkyl, aryl, arylalkyl, heteroaryl, or heteroarylalkyl, which group is unsubstituted or is substituted with one or more substituents.

“Ester” refers to a group such as —C(═O)OR⁴⁷, alternatively illustrated as —C(O)OR⁴⁷, wherein R⁴⁷ is selected from alkyl, cycloalkyl, cycloalkylalkyl, heterocycloalkyl, heterocycloalkylalkyl, aryl, arylalkyl, heteroaryl, and heteroarylalkyl, any of which is unsubstituted or is substituted with one or more substituents.

“Ether” refers to the group -alkyl-O-alkyl, where the term alkyl is as defined herein.

“Sulfanyl” refers to —SR⁴⁸, wherein R⁴⁸ is selected from alkyl, cycloalkyl, cycloalkylalkyl, heterocycloalkyl, heterocycloalkylalkyl, aryl, arylalkyl, heteroaryl, and heteroarylalkyl, any of which is unsubstituted or is substituted with one or more substituents. For example, —SR⁴⁸, wherein R⁴⁸ is an alkyl is an alkylsulfanyl.

“Sulfonyl” refers to —S(O)₂—, which may have various substituents to form different sulfonyl groups including sulfonic acids, sulfonamides, sulfonate esters, and sulfones. For example, —S(O)₂R⁴⁹, wherein R⁴⁹ is an alkyl refers to an alkylsulfonyl. In certain embodiments of —S(O)₂R⁴⁹, R⁴⁹ is selected from alkyl, cycloalkyl, cycloalkylalkyl, heterocycloalkyl, heterocycloalkylalkyl, aryl, arylalkyl, heteroaryl, and heteroarylalkyl, any of which is unsubstituted or is substituted with one or more substituents.

“Sulfinyl” refers to —S(O)—, which may have various substituents to form different sulfinyl groups including sulfinic acids, sulfinamides, and sulfinyl esters. For example, —S(O)R⁵⁰, wherein R⁵⁰ is an alkyl refers to an alkylsulfinyl. In certain embodiments of —S(O)R⁵⁰, R⁵⁰ is selected from alkyl, cycloalkyl, cycloalkylalkyl, heterocycloalkyl, heterocycloalkylalkyl, aryl, arylalkyl, heteroaryl, and heteroarylalkyl, any of which is unsubstituted or is substituted with one or more substituents.

“Silyl” refers to Si, which may have various substituents, for example —SiR⁵¹R⁵¹R⁵¹, where each R⁵¹ is independently selected from alkyl, cycloalkyl, cycloalkylalkyl, heterocycloalkyl, heterocycloalkylalkyl, aryl, arylalkyl, heteroaryl, and heteroarylalkyl, any of which is unsubstituted or is substituted with one or more substituents. As defined herein, any heterocycloalkyl or heteroaryl group present in a silyl group has from 1 to 3 heteroatoms selected independently from O, N, and S.

“TMS” refers to trimethylsilyl (—Si(CH₃)₃). “Amino” or “amine” refers to the group —N⁺R⁵²R⁵² or —N+R⁵²R⁵²R⁵², wherein each R⁵² is independently selected from hydrogen and a group selected from alkyl, cycloalkyl, heterocycloalkyl, alkyloxy, aryl, heteroaryl, heteroarylalkyl, acyl, —C(O)—O-alkyl, sulfanyl, sulfinyl, sulfonyl, and the like, which group is unsubstituted or is substituted with one or more substituents. Exemplary amino groups include, but are not limited to, dimethylamino, diethylamino, trimethylammonium, triethylammonium, methylsulfonylamino, furanyl-oxy-sulfamino, and the like.

“Amide” refers to a group such as —C(═O)NR⁵³R⁵³, wherein each R⁵³ is independently selected from H and a group selected from alkyl, cycloalkyl, cycloalkylalkyl, heterocycloalkyl, heterocycloalkylalkyl, aryl, arylalkyl, heteroaryl, and heteroarylalkyl, any of which is unsubstituted or is substituted with one or more substituents.

“Carbamate” refers to a group such as —O—C(═O)NR⁵³R⁵³ or —NR⁵³—C(═O)OR³, wherein each R⁵³ is independently selected from H and a group selected from alkyl, cycloalkyl, cycloalkylalkyl, heterocycloalkyl, heterocycloalkylalkyl, aryl, arylalkyl, heteroaryl, and heteroarylalkyl, any of which is unsubstituted or is substituted with one or more substituents.

“Sulfonamide” refers to —S(O)₂NR⁵⁴R⁵⁴, wherein each R⁵⁴ is independently selected from H and a group selected from alkyl, heteroalkyl, heteroaryl, heterocycle, alkenyl, alkynyl, arylalkyl, heteroarylalkyl, heterocyclylalkyl, alkylene-C(O)—OR⁵⁵, or alkylene-O—C(O)—OR⁵⁵, any of which is unsubstituted or is substituted with one or more substituents, where R⁵⁵ is selected from H, alkyl, heteroalkyl, cycloalkyl, heterocyclyl, aryl, heteroaryl, alkenyl, alkynyl, arylalkyl, heterocycloalkyl, heteroarylalkyl, amino, and sulfinyl.

“Adamantyl” refers to a compound of structural formula:

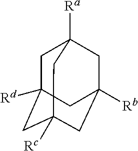

where R^{a}, R^{b}, R^{c}, and R^{d} are selected from H and a substituent (e.g., as defined herein). In some embodiments, each of R^{a}, R^{b}, R^{c}, and R^{d} are H. In some embodiments, at least one of R^{a}, R^{b}, R^{c}, and R^{d} is not H. In certain embodiments, adamantyl includes substituted adamantyl, e.g., 1- or 2-adamantyl, substituted by one or more substituents, including alkyl, halo, —OH, —NH₂, and alkoxy. Examples of adamantyl derivatives include methyladamantane, haloadamantane, hydroxyadamantane, and aminoadamantane (e.g., amantadine).

“N-protecting group” as used herein refers to those groups intended to protect a nitrogen atom against undesirable reactions during synthetic procedures. Examples of N-protecting groups include, but are not limited to, acyl groups such acetyl and t-butylacetyl; pivaloyl; alkoxycarbonyl groups such as methyloxycarbonyl and t-butyloxycarbonyl (Boc); aryloxycarbonyl groups such as benzyloxycarbonyl (Cbz) and fluorenylmethoxycarbonyl (Fmoc); and aroyl groups such as benzoyl. N-protecting groups are described in, e.g., Greene's Protective Groups in Organic Synthesis' 5th Edition, P. G. M. Wuts, ed., Wiley (2014).

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Substituted” as used herein means one or more hydrogen atoms of the group is replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, —OR⁵⁶ (e.g., hydroxyl, alkyloxy (e.g., methoxy, ethoxy, and propoxy), ether, ester, carbamate, etc.), hydroxyalkyl, —C(O)O-alkyl, —O-alkyl-O-alkyl, haloalkyl, alkyl-O-alkyl, SR⁵⁶ (e.g., —SH, —S-alkyl, —S-aryl, —S-heteroaryl, arylalkyl-S—, etc.), S⁺R⁵⁶, S(O)R⁵⁶, SO₂R⁵⁶, NR⁵⁶R⁵⁷ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, —SH, carboxy, aldehyde, keto, carboxylic acid, ester, amide, imine, and imide, including seleno and thio derivatives thereof, wherein each R⁵⁶ and R⁵⁷ are independently alkyl, alkenyl, alkynyl, heteroalkyl, cycloalkyl, cycloalkylalkyl, heterocycloalkyl, heterocycloalkylalkyl, aryl, arylalkyl, heteroaryl, or heteroarylalkyl, and wherein each of the substituents can be optionally further substituted. In embodiments in which a functional group with an aromatic carbon ring is substituted, such substitutions will typically number less than about 10 substitutions, or about 1 to 5, with about 1 or 2 substitutions in certain embodiments. In certain embodiments, a single carbon atom may bear one or more substituents. For example, a carbon atom may have one, two, or three substituents. As an example, a haloalkyl group such as —CF₃ may be alternatively described as an alkyl group (e.g., a methylene) having three fluoro substituents. Where a carbon atom bears two or more substituents, they may be the same or different.

“Pharmaceutically acceptable salt” is meant to include salts of the active compounds which are prepared with relatively nontoxic acids or bases, depending on the particular substituents found on the compounds described herein. When compounds as disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds as disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17th Ed., Mack Publishing Company, Easton, Pa., (1985) and Journal of Pharmaceutical Science, 66:2 (1977), each of which is incorporated herein by reference in its entirety.

“Pharmaceutically acceptable carrier” or “pharmaceutically acceptable excipient” refers to an excipient, carrier or adjuvant that can be administered to a subject, together with at least one compound, and which does not destroy the pharmacological activity thereof and is generally safe, nontoxic and neither biologically nor otherwise undesirable when administered in doses sufficient to deliver a therapeutic amount of the agent.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, e.g., a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds as disclosed herein, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable, and includes atropisomers. The present disclosure contemplates various stereoisomers and mixtures thereof and includes “enantiomers,” which refers to two stereoisomers whose molecules are non-superimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

### 2. Compounds

### Embodiment A

In certain embodiments, provided herein is a compound of Formula A-I, or a tautomer, stereoisomer, mixture of stereoisomers, isotopically enriched analog, or pharmaceutically acceptable salt thereof:

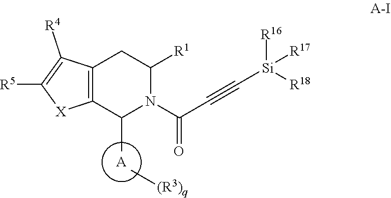

wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R⁵, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, that at least one of R¹⁶, R¹⁴, and R¹⁸ is other than unsubstituted methyl.

In certain embodiments, provided herein is a compound of Formula A-I, or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R′)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, the compound is not a compound of Table A-1B.

TABLE A-1B
Structures

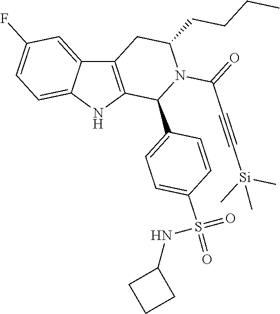 [SMILES: CCCC[C@H]1Cc2c([nH]c3ccc(F)cc23)[C@H](c2ccc(S(=O)(=O)NC3CCC3)cc2)N1C(=O)C#C[Si](C)(C)C]

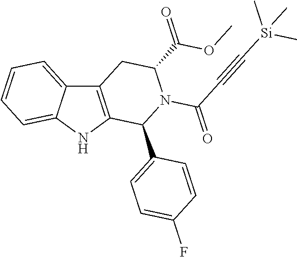 [SMILES: COC(=O)[C@H]1Cc2c([nH]c3ccccc23)[C@H](c2ccc(F)cc2)N1C(=O)C#C[Si](C)(C)C]

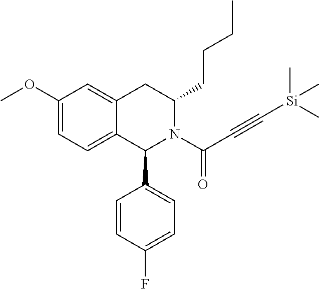

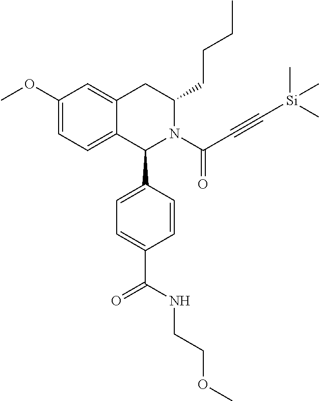

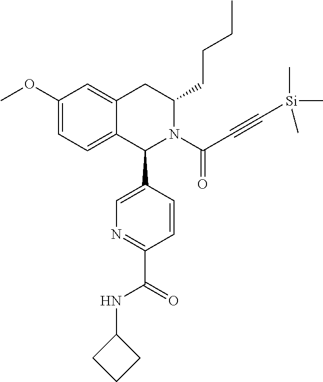

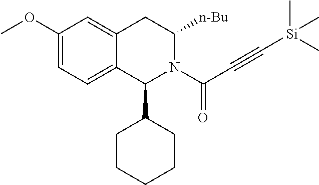

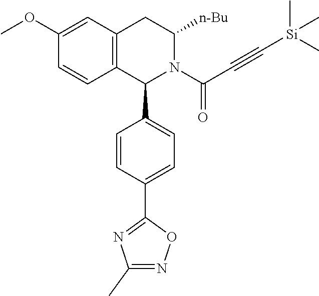

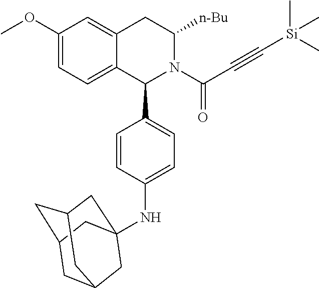

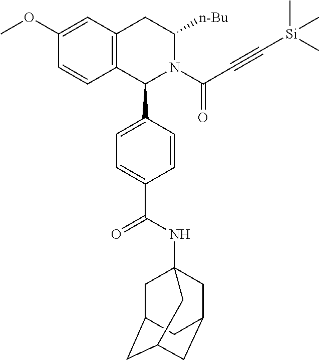

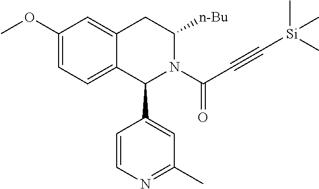

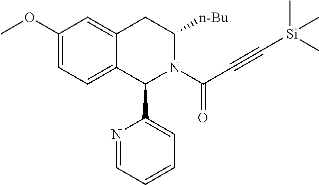

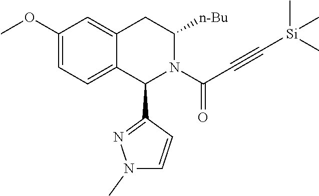

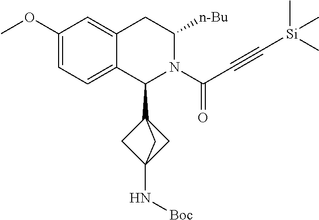

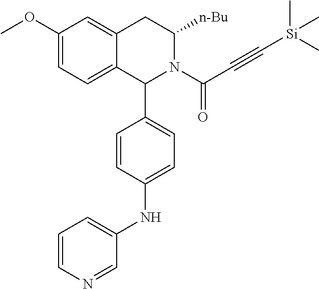

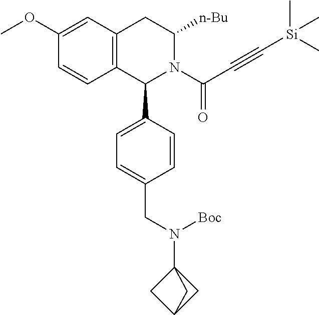

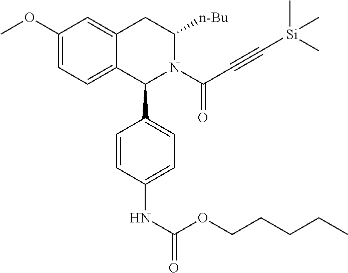

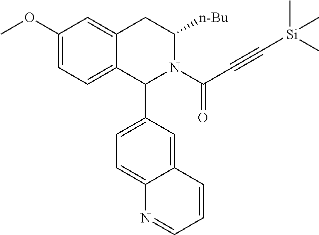

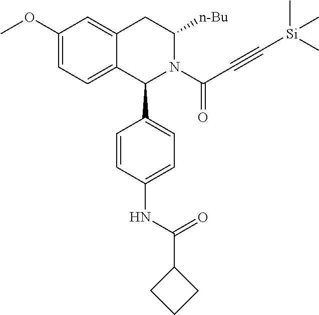

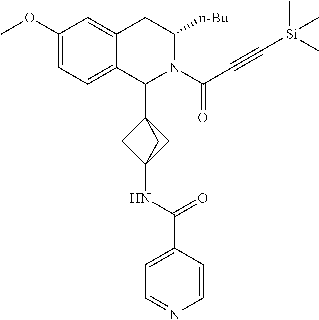

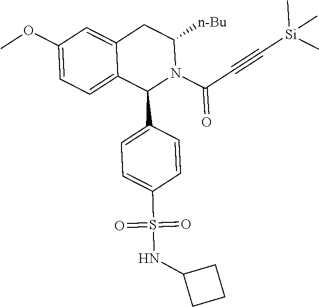

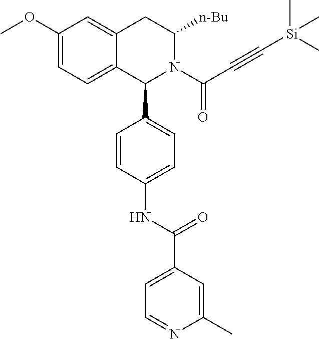

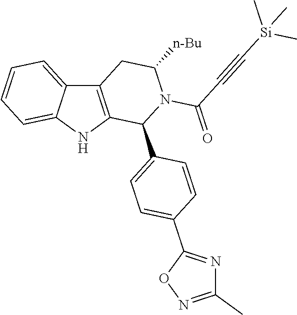

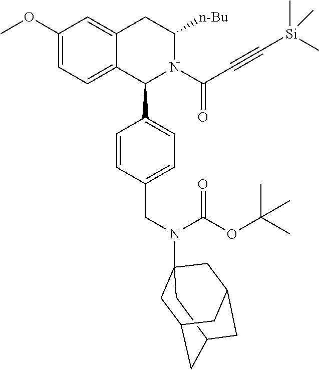

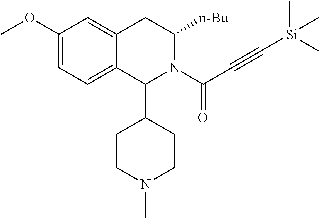

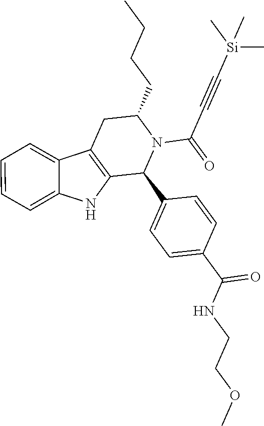 [SMILES: CCCC[C@H]1Cc2c([nH]c3ccccc23)[C@H](c2ccc(C(=O)NCCOC)cc2)N1C(=O)C#C[Si](C)(C)C]

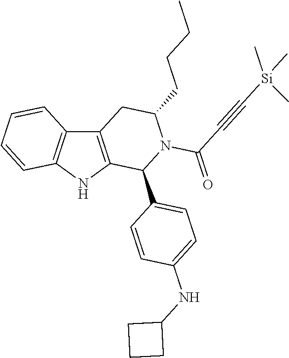 [SMILES: CCCC[C@H]1Cc2c([nH]c3ccccc23)[C@H](c2ccc(NC3CCC3)cc2)N1C(=O)C#C[Si](C)(C)C]

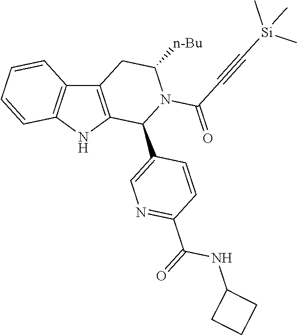

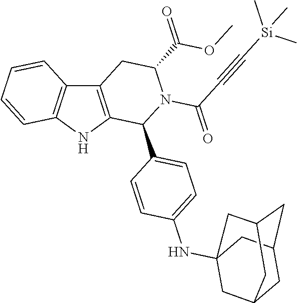 [SMILES: COC(=O)[C@H]1Cc2c([nH]c3ccccc23)[C@H](c2ccc(NC34CC5CC(CC(C5)C3)C4)cc2)N1C(=O)C#C[Si](C)(C)C]

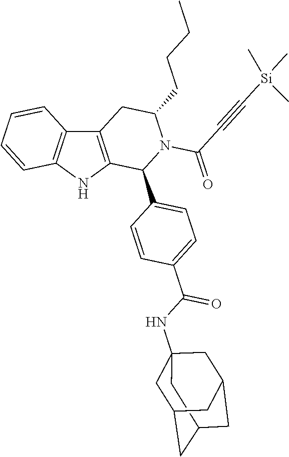 [SMILES: CCCC[C@H]1Cc2c([nH]c3ccccc23)[C@H](c2ccc(C(=O)NC34CC5CC(CC(C5)C3)C4)cc2)N1C(=O)C#C[Si](C)(C)C]

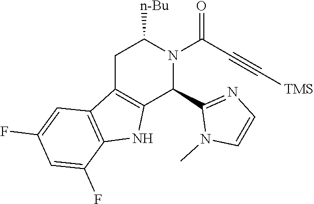

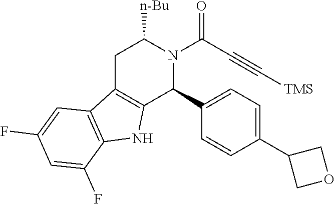

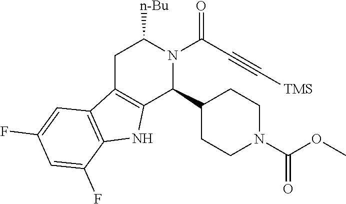

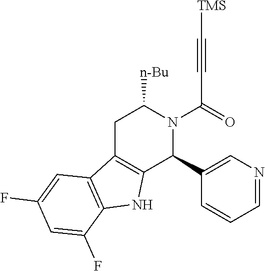

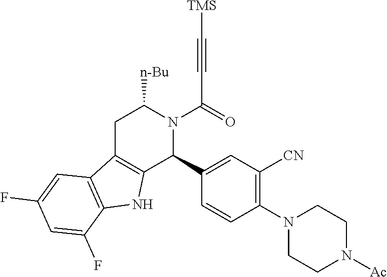

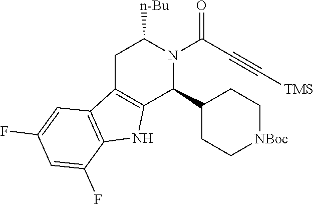

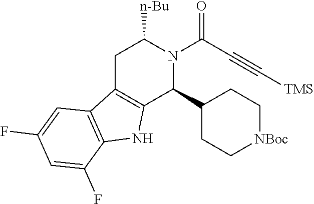

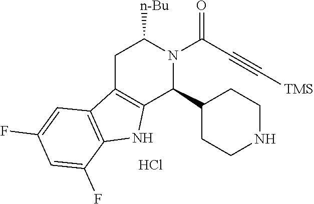

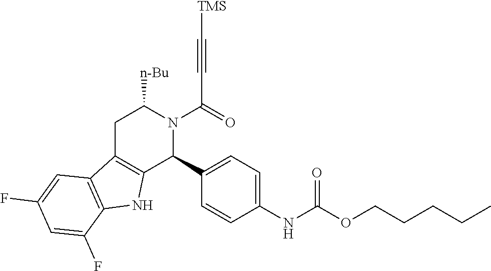

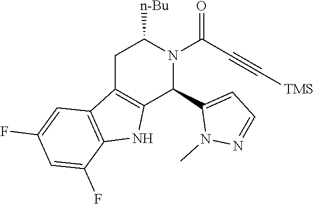

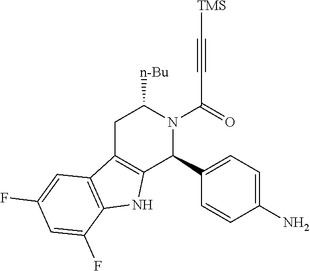

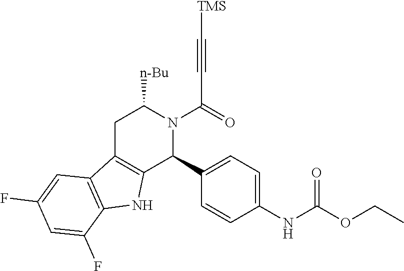

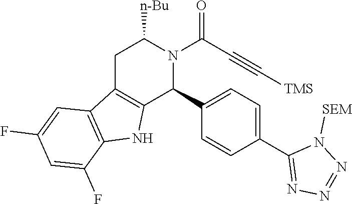

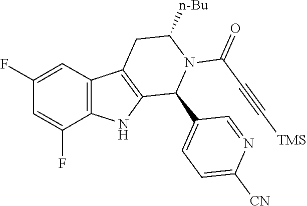

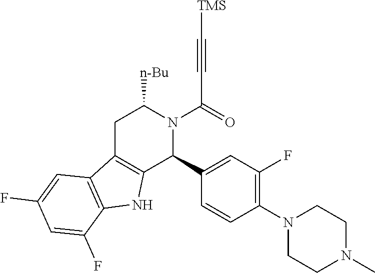

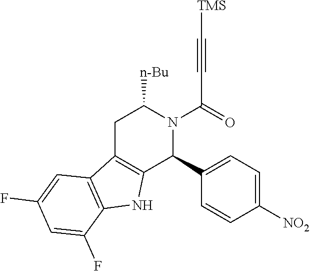

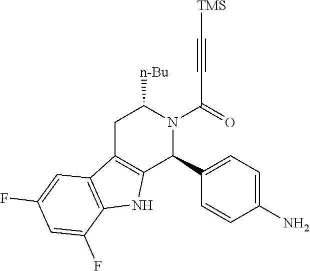

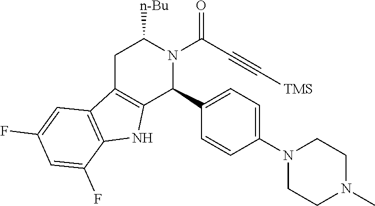

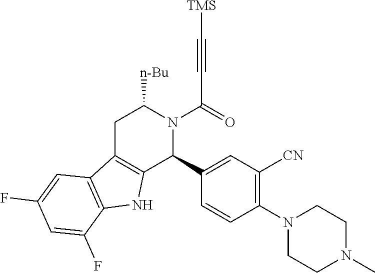

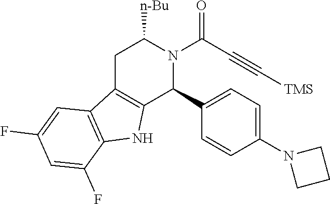

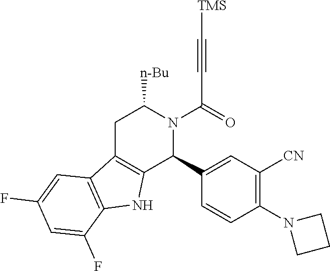

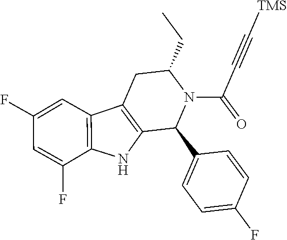 [SMILES: CC[C@H]1Cc2c([nH]c3c(F)cc(F)cc23)[C@H](c2ccc(F)cc2)N1C(=O)C#C[Si](C)(C)C]

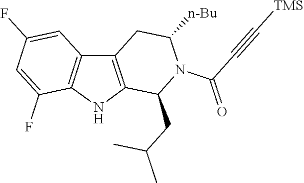

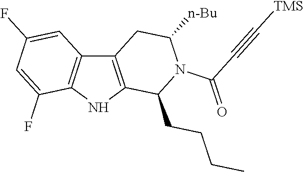

In certain embodiments, provided herein is a compound of Formula A-I, or a tautomer, stereoisomer, mixture of stereoisomers, isotopically enriched analog, or pharmaceutically acceptable salt thereof:

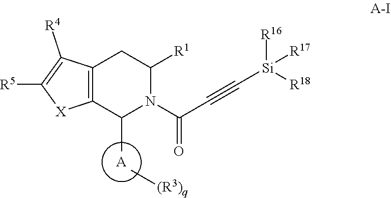

wherein:
X is —NR²²—, —O—, —S—, —N═CR⁹—, —CR⁹═CR⁹—, —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R is independently unsubstituted or substituted with one to three R¹⁰; or
when X is —NR²²—, —O—, or —S—, then R⁴ and R, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷ together with the nitrogen atom to which they are attached, form a 4 to 7 membered heterocyclyl, wherein each R⁷ or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or substituted with one to three R¹⁰.

In certain embodiments, provided herein is a compound of Formula A-I, or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R′)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷ together with the nitrogen atom to which they are attached, form a 4 to 7 membered heterocyclyl, wherein each R⁷ or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or substituted with one to three R¹⁰.

In certain embodiments, provided herein is a compound of Formula A-IA, or a tautomer, stereoisomer, mixture of stereoisomers, isotopically enriched analog, or pharmaceutically acceptable salt thereof:

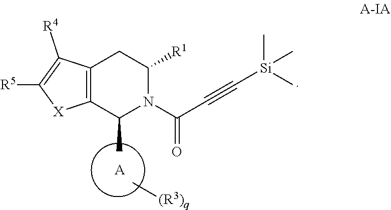

In certain embodiments, provided herein is a compound of Formula A-IB, or a tautomer, stereoisomer, mixture of stereoisomers, isotopically enriched analog, or pharmaceutically acceptable salt thereof:

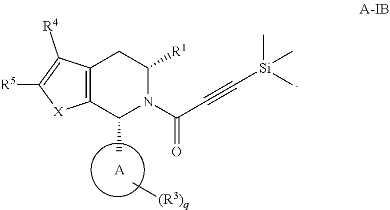

In certain embodiments, X is —NR²²—, —O—, or —S—.

In certain embodiments, X is —NH—.

In certain embodiments, X is —N═CR⁹—, —CR⁹═CR⁹—, or —CR⁹═N—.

In certain embodiments, X is —CR⁹═CR⁹—.

In certain embodiments, X is —CH═CH—.

In certain embodiments, provided herein is a compound of Formula A-I, or a tautomer, stereoisomer, mixture of stereoisomers, isotopically enriched analog, or pharmaceutically acceptable salt thereof:

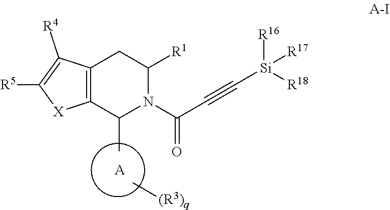

wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹⁵ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or substituted with one to three R¹⁰;
R¹⁷ is hydrogen or C₁-C₆ alkyl that is unsubstituted or substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, provided herein is a compound of Formula A-I, or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹⁵ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or substituted with one to three R¹⁰;
R¹⁷ is hydrogen or C₁-C₆ alkyl that is unsubstituted or substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, that at least one of R¹⁶, R¹⁴, and R¹⁸ is other than unsubstituted methyl.

In certain embodiments, each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently further substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently further substituted with one to three R¹¹; and
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently further substituted with one to three R¹¹.

In certain embodiments, the compound is not a compound of Table A-1C.

TABLE A-1C
Structures

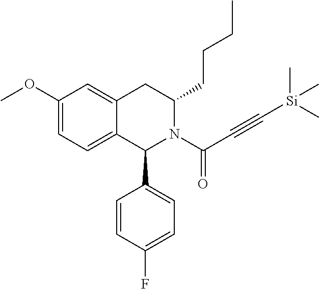

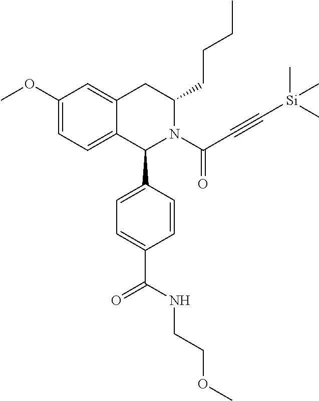

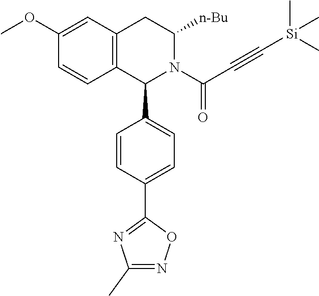

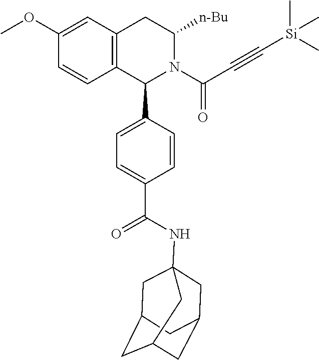

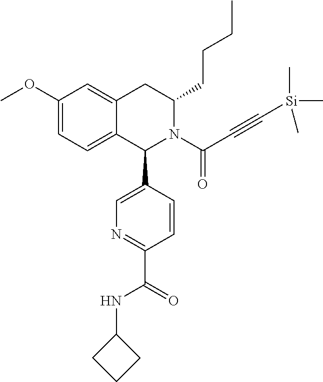

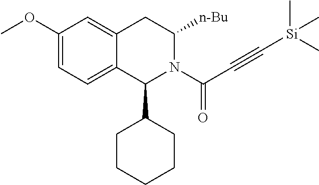

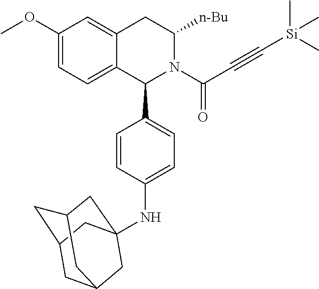

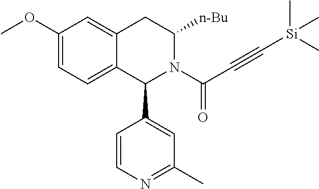

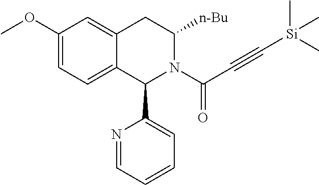

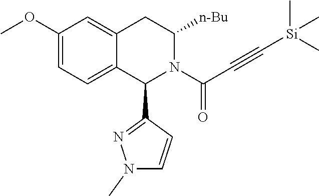

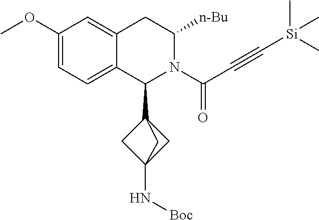

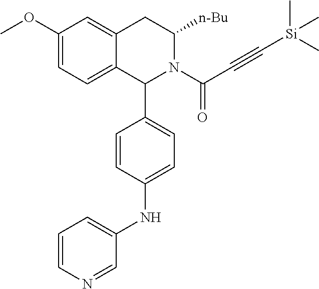

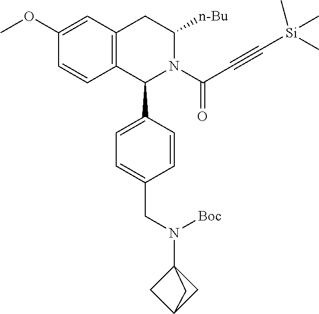

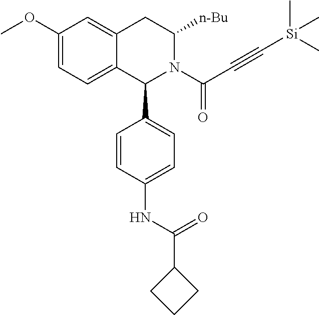

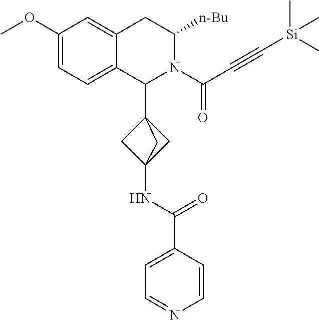

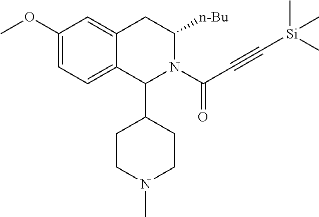

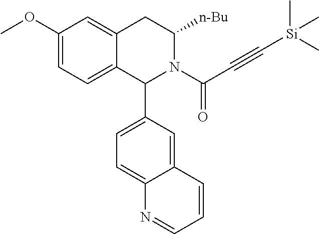

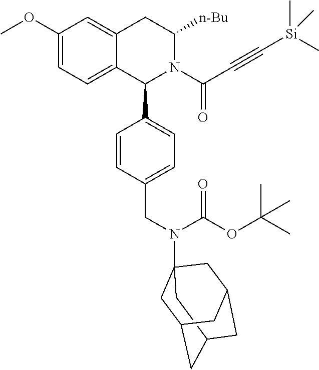

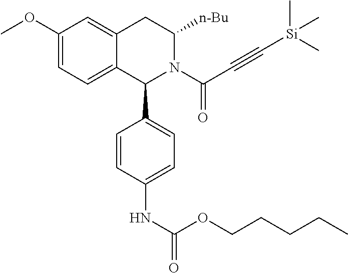

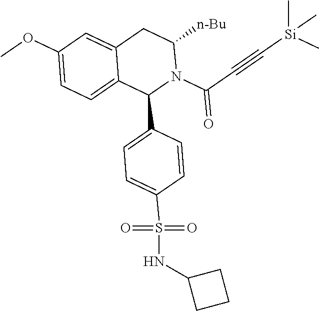

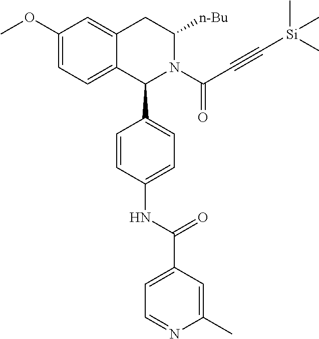

In certain embodiments, provided herein is a compound of Formula A-II, or a tautomer, stereoisomer, mixture of stereoisomers, isotopically enriched analog, or pharmaceutically acceptable salt thereof:

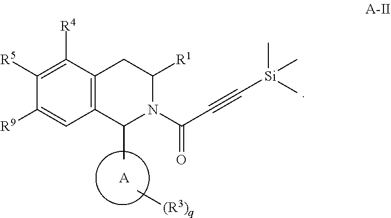

In certain embodiments, provided herein is a compound of Formula A-I, or a tautomer, stereoisomer, mixture of stereoisomers, isotopically enriched analog, or pharmaceutically acceptable salt thereof:

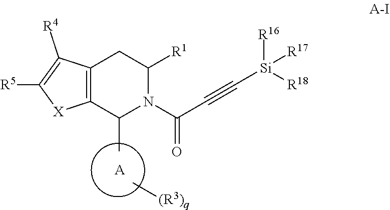

wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)R¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, provided herein is a compound of Formula A-I, or a tautomer, stereoisomer, mixture of stereoisomers, isotopically enriched analog, or pharmaceutically acceptable salt thereof:

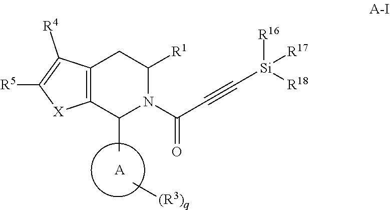

wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R is independently unsubstituted or substituted with one to three R¹⁰; or
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆ alkyl, C₂-C₆ alkenyl, C₂-C₆ alkynyl, or C₃-C₁₀ cycloalkyl, wherein each C₁-C₆ alkyl, C₂-C₆ alkenyl, C₂-C₆ alkynyl, or C₃-C₁₀ cycloalkyl of R⁴ is independently unsubstituted or substituted with one to three R¹⁰.

In certain embodiments, R⁴ and R⁵ are each independently hydrogen, halo, —CN, —OH, —OR⁸, C₁-C₆ alkyl, C₂-C₆ alkynyl, or C₃-C₁₀ cycloalkyl, wherein each C₁-C₆ alkyl, C₂-C₆ alkynyl, or C₃-C₁₀ cycloalkyl of R⁴ is independently unsubstituted or substituted with one to three R¹⁰.

In certain embodiments, provided herein is a compound of Formula A-III, or a tautomer, stereoisomer, mixture of stereoisomers, isotopically enriched analog, or pharmaceutically acceptable salt thereof:

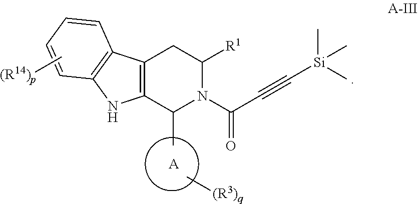

wherein p is 0, 1, 2, or 3.

In certain embodiments, p is 0. In certain embodiments, p is 0 or 1. In certain embodiments, p is 1 or 2. In certain embodiments, p is 1, 2, or 3. In certain embodiments, p is 1. In certain embodiments, p is 2.

In certain embodiments, p is 3.

In certain embodiments, each R¹⁴ is independently halo, —CN, —OH, —OR⁸, C₁-C₆ alkyl, C₂-C₆ alkynyl, or C₃-C₁₀ cycloalkyl.

In certain embodiments, ring A is aryl or heteroaryl. In certain embodiments, ring A is a monocyclic aryl or monocyclic heteroaryl. In certain embodiments, ring A is heterocyclyl. In certain embodiments, ring A is a 4 to 7 membered heterocyclyl. In certain embodiments, ring A is aryl. In certain embodiments, ring A is phenyl. In certain embodiments, ring A is heteroaryl. In certain embodiments, ring A is pyridyl. In certain embodiments, ring A is phenyl, pyridyl, piperidinyl, piperazinyl, or morpholinyl.

In certain embodiments, ring A is aryl or heteroaryl, any of which is substituted by one to three R³.

In certain embodiments, ring A is aryl or heteroaryl, any of which is substituted by one to three R³, where at least one R³ is C₃-C₁₀ cycloalkyl, heterocyclyl, aryl, or heteroaryl, wherein any C₃-C₁₀ cycloalkyl, heterocyclyl, aryl, and heteroaryl of R³ is unsubstituted or is substituted with one to three R¹⁰.

In certain embodiments, ring A is aryl or heteroaryl, any of which is substituted by two or three R³.

In certain embodiments, ring A is aryl or heteroaryl, any of which is substituted by two or three R³, wherein at least one R³ is halo.

In certain embodiments, ring A is:

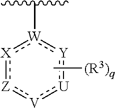

wherein 0 to 3 of U, V, W, X, Y, and Z is independently N, S, or O, and the remaining variables are CH or CR³ and each [symbol] independently represents a single or double bond, which comply with valency requirements based on U, V, W, X, Y and Z.

In certain embodiments, ring A is:

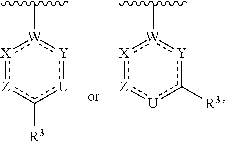

wherein 1 to 3 of U, W, X, Y, and Z is N, S, or O, and the remaining variables are CH or CR³ and [symbol] represents a single or double bond, which comply with valency requirements based on U, W, X, Y and Z.

In certain embodiments, ring A is aryl or heteroaryl. In certain embodiments, ring A is a monocyclic aryl or monocyclic heteroaryl. In certain embodiments, ring A is heterocyclyl. In certain embodiments, ring A is a 4 to 7 membered heterocyclyl. In certain embodiments, ring A is aryl. In certain embodiments, ring A is phenyl. In certain embodiments, ring A is heteroaryl. In certain embodiments, ring A is pyridyl. In certain embodiments, ring A is pyrazolyl. In certain embodiments, ring A is phenyl, pyridyl, piperidinyl, piperazinyl, or morpholinyl.

In certain embodiments, ring A is aryl or heteroaryl, any of which is substituted by one to three R³. In certain embodiments, ring A is aryl or heteroaryl, any of which is substituted by one to three R³, where at least one R³ is C₃-C₁₀ cycloalkyl, heterocyclyl, aryl, or heteroaryl, wherein any C₃-C₁₀ cycloalkyl, heterocyclyl, aryl, and heteroaryl of R³ is unsubstituted or is substituted with one to three R¹⁰.

In certain embodiments, ring A is aryl or heteroaryl, any of which is substituted by two or three R³. In certain embodiments, ring A is aryl or heteroaryl, any of which is substituted by two or three R³, wherein at least one R³ is halo.

In certain embodiments, ring A is cyclohexyl. In certain embodiments, ring A is C₄-C₁₀ cycloalkyl. In certain embodiments, ring A is a C₄-C₇ cycloalkyl. In certain embodiments, ring A is bicyclo[1.1.1]pentanyl. In certain embodiments, ring A is selected from cyclobutyl, cyclopentyl, cyclohexyl, and cycloheptyl.

In certain embodiments, ring A is:

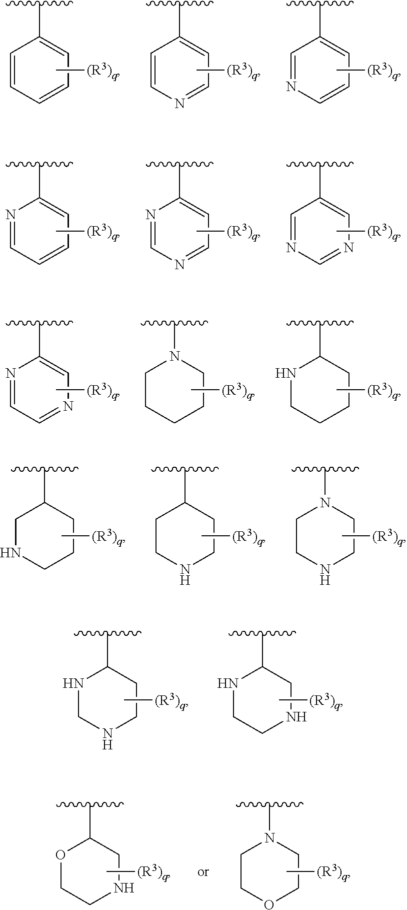

where q and each R³ is independently as defined herein.

In certain embodiments, ring A is:

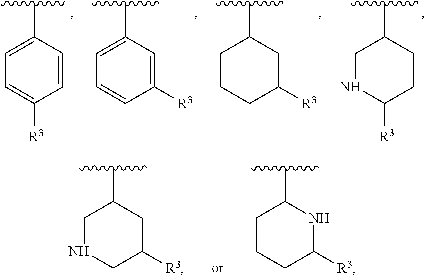

where R³ is independently as defined herein.

In certain embodiments, ring A is a bridged bicyclic ring selected from:

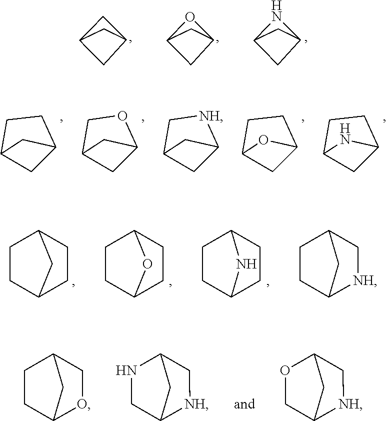

wherein each is substituted with one to three R³. In certain embodiments, ring A is a bridged bicyclic ring selected from:

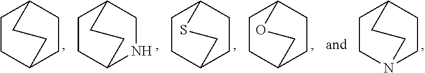

wherein each R³ is attached to a carbon atom on the bridged bicyclic ring.

In certain embodiments, ring A is:

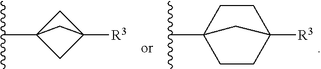

In certain embodiments, R¹ is C₁-C₆ alkyl, C₂-C₆ alkenyl, C₂-C₆ alkynyl, C₁-C₆ haloalkyl, C₃-C₁₀ cycloalkyl, —CN, —C(O)OR⁶, —C(O)N(R⁷)₂, —N(R⁷)₂, —OR⁸, or —C₁-C₆ alkyl-OR⁷.

In certain embodiments, R¹ is —C(O)OR⁶ or —C(O)N(R⁷)₂.

In certain embodiments, R¹ is C₁-C₆ alkyl. In certain embodiments, R¹ is C₂-C₆ alkyl. In certain embodiments, R¹ is C₃-C₆ alkyl. In certain embodiments, R¹ is C₅-C₆ alkyl. In certain embodiments, R¹ is C₂-C₃ alkyl. In certain embodiments, R¹ is C₄-C₆ alkyl. In certain embodiments, R¹ is methyl. In certain embodiments, R¹ is n-butyl or i-butyl. In certain embodiments, R¹ is n-butyl. In certain embodiments, R¹ is C₃-C₁₀ cycloalkyl. In certain embodiments, R¹ is —C₁-C₆ alkyl-C₃-C₁₀ cycloalkyl. In certain embodiments, R¹ is C₃-C₁₀ cycloalkyl or —C₁-C₆ alkyl-C₃-C₁₀ cycloalkyl.

In certain embodiments, R¹ is —CH₂—R³⁶, wherein R³⁶ is C₁-C₅ alkyl, C₂-C₅ alkenyl, C₂-C₅ alkynyl, C₁-C₅ haloalkyl, or —C₁-C₅ alkyl-OR. In certain embodiments, R¹ is —CH₂—R³⁶, wherein R³⁶ is C₁-C₅ alkyl, C₂-C₅ alkenyl, C₂-C₅ alkynyl, C₁-C₅ haloalkyl, or —C₁-C₅ alkyl-OR⁸.

In certain embodiments, R¹ is C₂-C₆ alkenyl, C₂-C₆ alkynyl, C₁-C₆ haloalkyl, C₃-C₁₀ cycloalkyl, —CN, —OR⁸, —C(O)N(R⁷)₂—OC(O)R⁶, —S(O)₂R, —S(O)₂N(R⁷)₂, —S(O)N(R⁷)₂, —S(O)R⁸, —N(R⁷)₂, —NO₂, —C₁-C₆ alkyl-OR⁸, or —Si(R¹⁵)₃.

In certain embodiments, R¹ is other than methyl. In certain embodiments, R¹ is other than n-butyl. In certain embodiments, R¹ is other than —C(O)OR⁶. In certain embodiments, R¹ is other than —C(O)OCH₃.

In certain embodiments, at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

In certain embodiments, at least one R³ is —NHR⁸, —OH, —OR⁸, —S(O)₂R, —S(O)R⁸, —NR¹²C(O)R⁸, —NR¹²C(O)OR⁸, —OC(O)R⁸, or —OC(O)CHR⁸N(R¹²)₂.

In certain embodiments, at least one R³ is halo.

In certain embodiments, at least one R³ is —NHR⁸. In certain embodiments, at least one R³ is —N(R⁸)₂. In certain embodiments, q is 2, and one R³ is halo or —CN, and the other R³ is —N(R⁸)₂. In certain embodiments, q is 2, and one R³ is halo and the other R³ is —N(R⁸)₂. In certain embodiments, q is 3, and two R³ are independently halo and one R³ is —N(R⁸)₂.

In certain embodiments, at least one R³ is —NHR⁸ or —OR⁸.

In certain embodiments, R⁸ is C₃-C₁₀ cycloalkyl.

In certain embodiments, R⁸ is adamantyl.

In certain embodiments, at least one R³ is —C(O)OR⁶ or —C(O)R⁶.

In certain embodiments, at least one R³ is —S(O)₂N(R⁷)₂, —S(O)N(R⁷)₂, or —C(O)N(R⁷)₂.

In certain embodiments, at least one R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂.

In certain embodiments, each R³ is independently halo, —CN, —OH, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆ alkyl, C₃-C₁₀ cycloalkyl, heterocyclyl, heteroaryl, or —C₁-C₆ alkylheterocyclyl, wherein each C₁-C₆ alkyl, C₃-C₁₀ cycloalkyl, heterocyclyl, heteroaryl, or —C₁-C₆ alkylheterocyclyl of R³ is independently unsubstituted or substituted with one to three R¹⁰.

In certain embodiments, each R³ is independently halo, —CN, —OH, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆ alkyl, C₃-C₁₀ cycloalkyl, heterocyclyl, heteroaryl, or —C₁-C₆ alkylheterocyclyl, wherein each C₁-C₆ alkyl, C₃-C₁₀ cycloalkyl, heterocyclyl, heteroaryl, or —C₁-C₆ alkylheterocyclyl is independently unsubstituted or substituted with one to three substituents independently selected from —OR¹², —N(R¹²)₂, —S(O)₂R¹³, —OC(O)CHR¹²N(R¹²)₂, and C₁-C₆ alkyl that is unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, R⁴ is hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆ alkyl, C₂-C₆ alkenyl, C₂-C₆ alkynyl, or C₃-C₁₀ cycloalkyl, wherein each C₁-C₆ alkyl, C₂-C₆ alkenyl, C₂-C₆ alkynyl, or C₃-C₁₀ cycloalkyl of R⁴ is independently unsubstituted or substituted with one to three R¹⁰.

In certain embodiments, R⁴ is hydrogen, halo, —CN, —OH, —OR⁸, C₁-C₆ alkyl, C₂-C₆ alkynyl, or C₃-C₁₀ cycloalkyl; wherein each C₁-C₆ alkyl, C₂-C₆ alkynyl, or C₃-C₁₀ cycloalkyl of R⁴ is independently unsubstituted or substituted with one to three R¹⁰.

In certain embodiments, R⁴ is hydrogen, halo, —CN, —OH, —OR⁸, C₁-C₆ alkyl, C₂-C₆ alkynyl, or C₃-C₁₀ cycloalkyl.

In certain embodiments, R⁴ is hydrogen, halo, —CN, —OH, —OR⁸, C₁-C₆ alkyl, or C₂-C₆ alkynyl, wherein the C₁-C₆ alkyl of R⁴ is unsubstituted or substituted with one to three R¹⁰.

In certain embodiments, R⁴ is hydrogen, halo, —CN, —OH, —OR⁸, C₁-C₆ alkyl, C₂-C₆ alkynyl, wherein the C₁-C₆ alkyl of R⁴ is unsubstituted or substituted with one to three substituents independently selected from —OR¹², —N(R¹²)₂, —S(O)₂R¹³, —OC(O)CHR¹²N(R¹²)₂, and C₁-C₆ alkyl that is unsubstituted or substituted with one to three halo, —OR¹², —N(R¹²)₂, —Si(R¹²)₃, —C(O)OR¹², —NR¹C(O)OR¹², —OC(O)CHR¹²N(R¹²)₂, C₁-C₆ alkyl, or heterocyclyl; wherein

R⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, R⁴ is halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆ alkyl, C₂-C₆ alkenyl, C₂-C₆ alkynyl, or C₃-C₁₀ cycloalkyl, wherein each C₁-C₆ alkyl, C₂-C₆ alkenyl, C₂-C₆ alkynyl, or C₃-C₁₀ cycloalkyl of R⁴ is independently unsubstituted or substituted with one to three R¹⁰.

In certain embodiments, R⁴ is halo, —CN, —OH, —OR⁸, C₁-C₆ alkyl, C₂-C₆ alkynyl, or C₃-C₁₀ cycloalkyl, wherein each C₁-C₆ alkyl, C₂-C₆ alkynyl, or C₃-C₁₀ cycloalkyl of R⁴ is independently unsubstituted or substituted with one to three R¹⁰.

In certain embodiments, R⁴ is halo, —CN, —OH, —OR⁸, C₁-C₆ alkyl, C₂-C₆ alkynyl, or C₃-C₁₀ cycloalkyl.

In certain embodiments, R⁴ is halo, —CN, —OH, —OR⁸, C₁-C₆ alkyl, or C₂-C₆ alkynyl, wherein the C₁-C₆ alkyl of R⁴ is unsubstituted or substituted with one to three R¹⁰.

In certain embodiments, R⁴ is halo, —CN, —OH, —OR⁸, C₁-C₆ alkyl, C₂-C₆ alkynyl, wherein the C₁-C₆ alkyl of R⁴ is unsubstituted or substituted with one to three substituents independently selected from —OR¹², —N(R¹²)₂, —S(O)₂R¹³, —OC(O)CHR¹²N(R¹²)₂, and C₁-C₆ alkyl that is unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein

R⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, R⁵ is hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆ alkyl, C₂-C₆ alkenyl, C₂-C₆ alkynyl, or C₃-C₁₀ cycloalkyl, wherein each C₁-C₆ alkyl, C₂-C₆ alkenyl, C₂-C₆ alkynyl, or C₃-C₁₀ cycloalkyl of R⁵ is independently unsubstituted or substituted with one to three R¹⁰.

In certain embodiments, R⁵ is hydrogen, halo, —CN, —OH, —OR⁸, C₁-C₆ alkyl, C₂-C₆ alkynyl, or C₃-C₁₀ cycloalkyl, wherein each C₁-C₆ alkyl, C₂-C₆ alkynyl, or C₃-C₁₀ cycloalkyl of R⁵ is independently unsubstituted or substituted with one to three R¹⁰.

In certain embodiments, R⁵ is hydrogen, halo, —CN, —OH, —OR⁸, C₁-C₆ alkyl, C₂-C₆ alkynyl, or C₃-C₁₀ cycloalkyl.

In certain embodiments, R is hydrogen, halo, —CN, —OH, —OR⁸, C₁-C₆ alkyl, or C₂-C₆ alkynyl, wherein the C₁-C₆ alkyl of R⁵ is unsubstituted or substituted with one to three R¹⁰.

In certain embodiments, R⁵ is hydrogen, halo, —CN, —OH, —OR⁸, C₁-C₆ alkyl, C₂-C₆ alkynyl, wherein the C₁-C₆ alkyl of R⁵ is unsubstituted or substituted with one to three substituents independently selected from —OR¹², —N(R¹²)₂, —S(O)₂R¹³, —OC(O)CHR¹²N(R¹²)₂, and C₁-C₆ alkyl that is unsubstituted or substituted with one to three halo, —OR¹², —N(R¹²)₂, —Si(R¹²)₃, —C(O)OR¹², —NR¹C(O)OR¹², —OC(O)CHR¹²N(R¹²)₂, C₁-C₆ alkyl, or heterocyclyl; wherein
R⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl; wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, each R⁶ is independently hydrogen, C₁-C₆ alkyl, C₂-C₆ alkenyl, or —C₁-C₆ alkylC₃-C₁₀ cycloalkyl, wherein each R⁶ is independently further substituted with one to three R¹¹.

In certain embodiments, each R⁶ is independently hydrogen, C₁-C₆ alkyl, C₂-C₆ alkenyl, or —C₁-C₆ alkylC₃-C₁₀ cycloalkyl, wherein each R⁶ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
each R¹² is independently hydrogen, C₁-C₆ alkyl, or C₃-C₁₀ cycloalkyl.

In certain embodiments, each R⁷ is independently hydrogen, C₁-C₆ alkyl, C₃-C₁₀ cycloalkyl, heterocyclyl, heteroaryl, —C₁-C₆ alkylC₃-C₆ cycloalkyl, —C₁-C₆ alkylheterocyclyl, or two R⁷, together with the nitrogen atom to which they are attached, form a 4 to 7 membered heterocyclyl, wherein each R or ring formed thereby is independently further substituted with one to three R¹¹.

In certain embodiments, each R⁷ is independently hydrogen, C₁-C₆ alkyl, C₃-C₁₀ cycloalkyl, heterocyclyl, heteroaryl, —C₁-C₆ alkylC₃-C₆ cycloalkyl, —C₁-C₆ alkylheterocyclyl, or two R⁷, together with the nitrogen atom to which they are attached, form a 4 to 7 membered heterocyclyl, wherein each R or ring formed thereby is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
each R¹² is independently hydrogen, C₁-C₆ alkyl, or C₃-C₁₀ cycloalkyl.

In certain embodiments, each R⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R⁸ is independently further substituted with one to three R¹¹.

In certain embodiments, each R⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein each R¹² is independently hydrogen, C₁-C₆ alkyl, or C₃-C₁₀ cycloalkyl.

In certain embodiments, R⁸ is C₁-C₆ alkyl. In certain embodiments, R⁸ is C₃-C₁₀ cycloalkyl. In certain embodiments, R⁸ is —C₁-C₆ alkylC₃-C₁₀ cycloalkyl. In certain embodiments, R⁸ is C₁-C₆ alkyl, C₂-C₆ alkynyl, C₃-C₁₀ cycloalkyl, or —C₁-C₆ alkylC₃-C₁₀ cycloalkyl.

In certain embodiments, R⁹ is hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆ alkyl, C₂-C₆ alkenyl, C₂-C₆ alkynyl, or C₃-C₁₀ cycloalkyl, wherein each C₁-C₆ alkyl, C₂-C₆ alkenyl, C₂-C₆ alkynyl, or C₃-C₁₀ cycloalkyl of R⁹ is independently unsubstituted or substituted with one to three R¹⁰.

In certain embodiments, R⁹ is hydrogen, halo, —CN, —OH, —OR⁸, C₁-C₆ alkyl, C₂-C₆ alkynyl, or C₃-C₁₀ cycloalkyl, wherein each C₁-C₆ alkyl, C₂-C₆ alkynyl, or C₃-C₁₀ cycloalkyl of R⁹ is independently unsubstituted or substituted with one to three R¹⁰.

In certain embodiments, R⁹ is hydrogen, halo, —CN, —OH, —OR⁸, C₁-C₆ alkyl, C₂-C₆ alkynyl, or C₃-C₁₀ cycloalkyl.

In certain embodiments, R⁹ is hydrogen, halo, —CN, —OH, —OR⁸, C₁-C₆ alkyl, or C₂-C₆ alkynyl, wherein the C₁-C₆ alkyl of R⁵ is unsubstituted or is substituted with one to three R¹⁰.

In certain embodiments, R⁹ is hydrogen, halo, —CN, —OH, —OR⁸, C₁-C₆ alkyl, C₂-C₆ alkynyl, wherein the C₁-C₆ alkyl of R⁹ is unsubstituted or is substituted with one to three substituents independently selected from —OR¹², —N(R¹²)₂, —S(O)₂R¹³, —OC(O)CHR¹²N(R¹²)₂, and C₁-C₆ alkyl that is unsubstituted or is substituted with one to three halo, —OR¹², —N(R¹²)₂, —Si(R¹²)₃, —C(O)OR¹², —NR¹C(O)OR¹², —OC(O)CHR¹²N(R¹²)₂, C₁-C₆ alkyl, or heterocyclyl; wherein
R⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, R¹⁶ is C₁-C₆ alkyl substituted with one to three R¹⁰. In certain embodiments, R¹⁶ is C₁-C₆ alkyl this is unsubstituted or is substituted with one to three R¹⁰. In certain embodiments, R¹⁶ is C₁-C₆ alkyl this is unsubstituted or is substituted with one to three —OH, CN, heterocyclyl, or —OC(O)R¹².

In certain embodiments, each R¹⁰ is independently —OR¹², —N(R¹²)₂, —S(O)₂R¹³, —OC(O)CHR¹²N(R¹²)₂, or C₁-C₆ alkyl, wherein the C₁-C₆ alkyl, of R¹⁰ is independently unsubstituted or substituted with one to three R¹¹;
each R¹⁵ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and each R¹³ is independently C₁-C₆ alkyl or C₃-C₁₀ cycloalkyl.

In certain embodiments, each R¹⁵ is independently C₁-C₆ alkyl.

In certain embodiments, q is 0. In certain embodiments, q is 0 or 1. In certain embodiments, q is 1 or 2. In certain embodiments, q is 1, 2, or 3. In certain embodiments, q is 1. In certain embodiments, q is 2.

In certain embodiments, q is 3.

Also provided is a compound, or a tautomer, stereoisomer, mixture of stereoisomers, isotopically enriched analog, or pharmaceutically acceptable salt thereof, selected from Table A-1A:

TABLE A-1A
Selected compounds of the disclosure.

| Compound | |
|---|---|
| No. | Structure |
| A-1 | 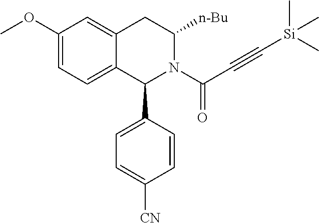 |
| A-3 | 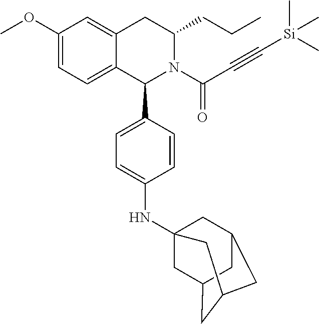 |
| A-4 | 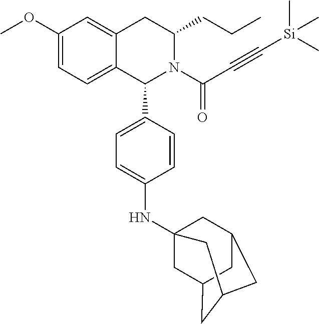 |
| A-5 | 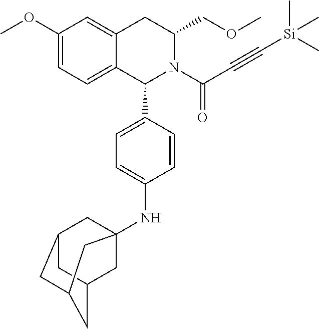 |
| A-7 | 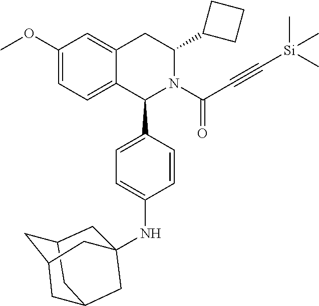 |
| A-8 | 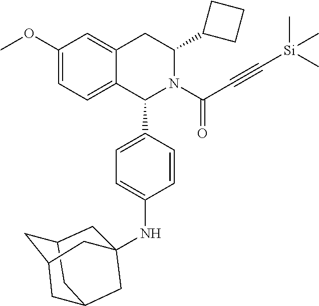 |
| A-9 | 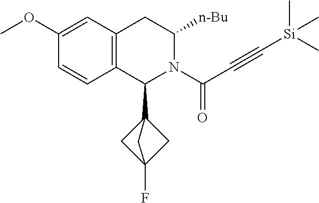 |
| A-10 | 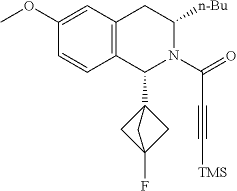 |
| A-11 | 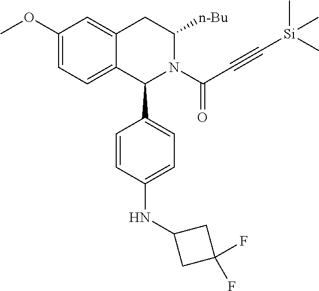 |
| A-12 | 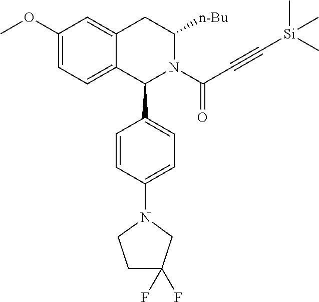 |
| A-13 | 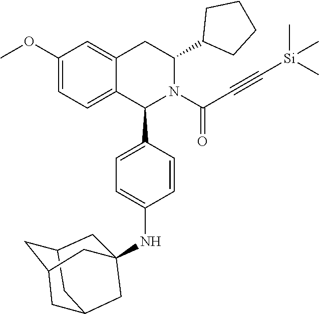 |
| A-14 | 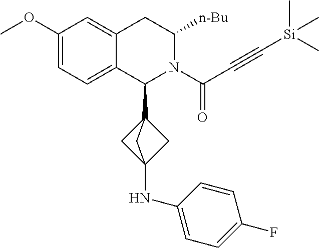 |
| A-15 | 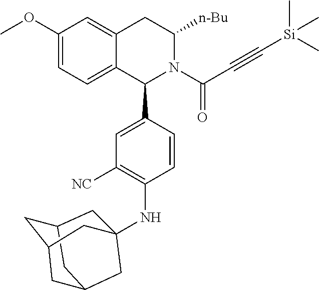 |
| A-16 | 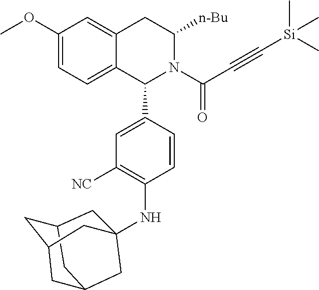 |
| A-17 | 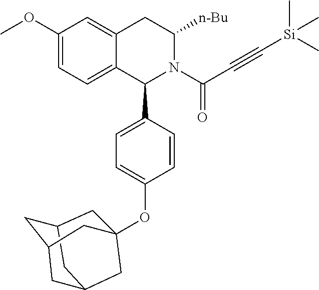 |
| A-18 | 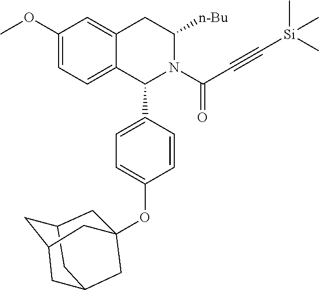 |
| A-19 | 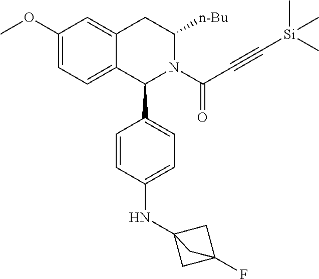 |
| A-20 | 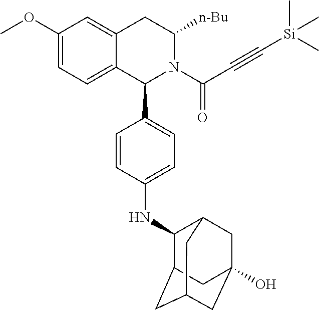 |
| A-21 | 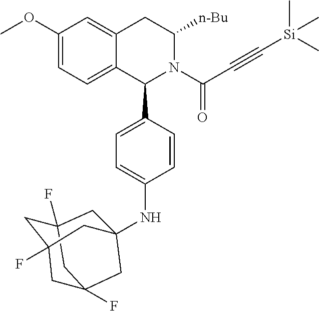 |
| A-22 | 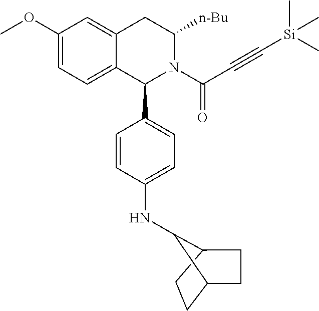 |
| A-23 | 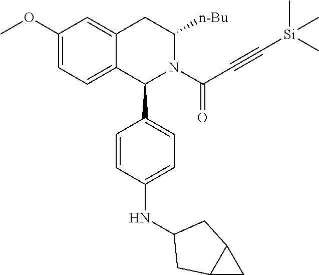 |
| A-24 | 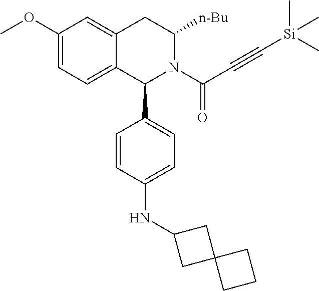 |
| A-25 | 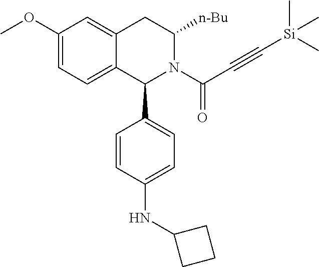 |
| A-26 | 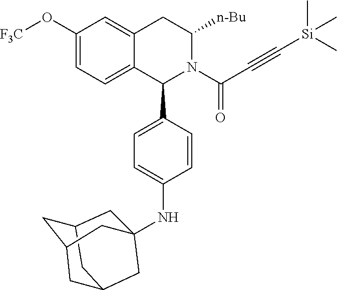 |
| A-27 | 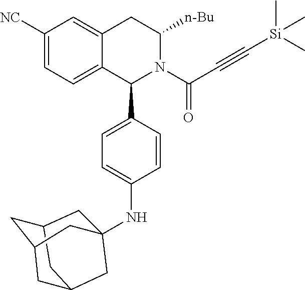 |
| A-28 | 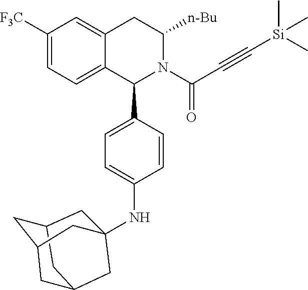 |
| A-29 | 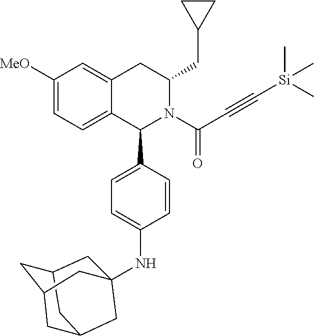 [SMILES: COc1ccc2c(c1)C[C@H](CC1CC1)N(C(=O)C#C[Si](C)(C)C)[C@H]2c1ccc(NC23CC4CC(CC(C4)C2)C3)cc1] |
| A-30 | 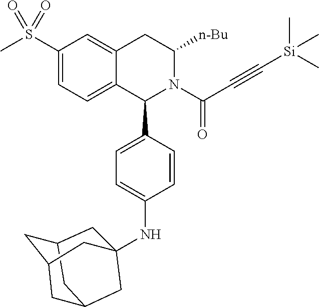 |
| A-31 | 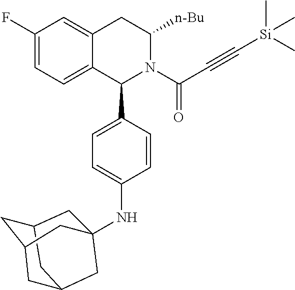 |
| A-32 | 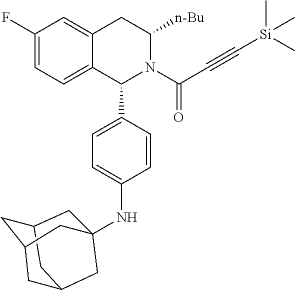 |
| A-33 | 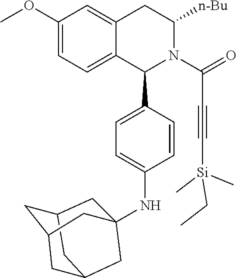 |
| A-34 | 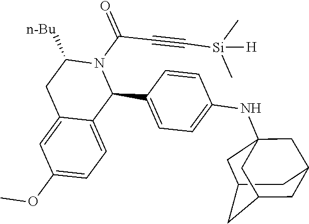 |
| A-35 | 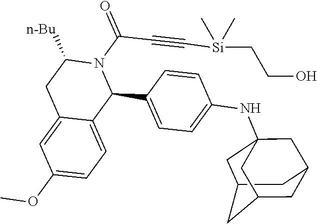 |
| A-36 | 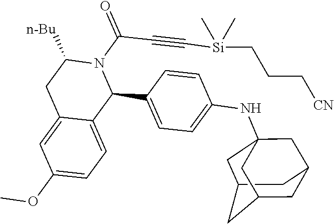 |
| A-37 | 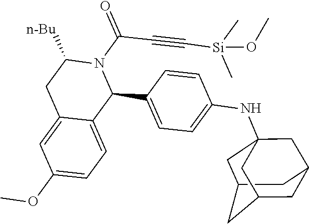 |
| A-38 | 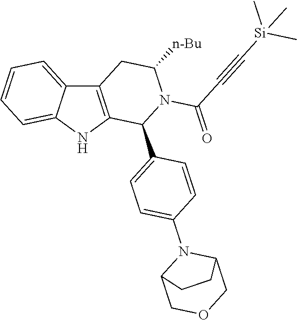 |
| A-39 | 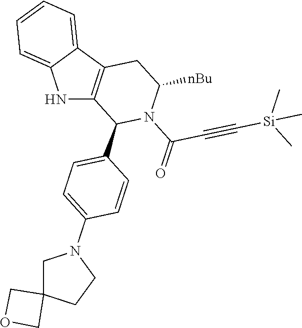 |
| A-40 | 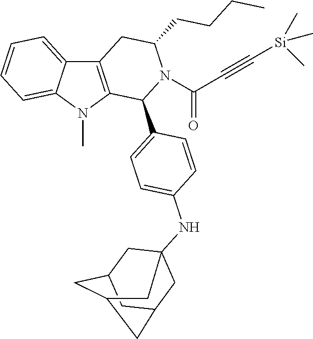 [SMILES: CCCC[C@H]1Cc2c(n(C)c3ccccc23)[C@H](c2ccc(NC34CC5CC(CC(C5)C3)C4)cc2)N1C(=O)C#C[Si](C)(C)C] |

Also provided is a compound, or a tautomer, stereoisomer, mixture of stereoisomers, isotopically enriched analog, or pharmaceutically acceptable salt thereof, selected from Table A-2A:

TABLE A-2A
Selected compounds of the disclosure.

| Compound | |
|---|---|
| No. | Structure |
| A-41 | 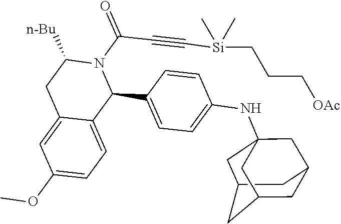 |
| A-42 | 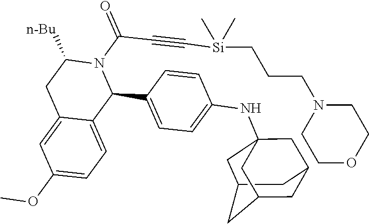 |
| A-43 | 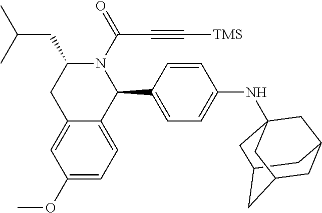 [SMILES: COc1ccc2c(c1)C[C@H](CC(C)C)N(C(=O)C#C[Si](C)(C)C)[C@H]2c1ccc(NC23CC4CC(CC(C4)C2)C3)cc1] |
| A-44 | 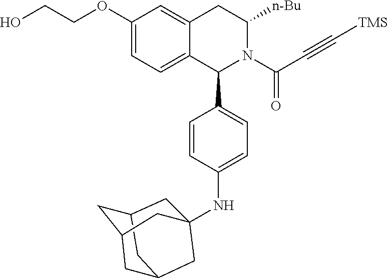 |
| A-45 | 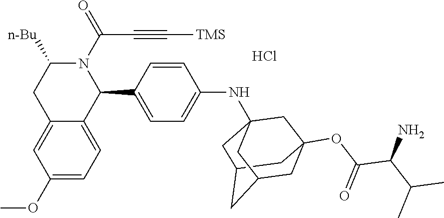 |
| A-46 | 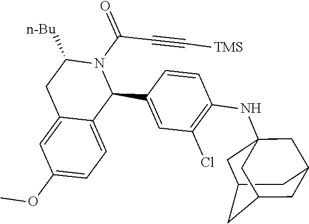 |
| A-47 | 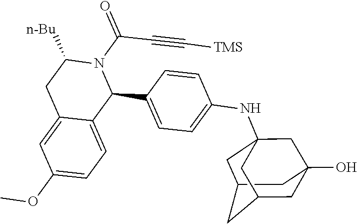 |
| A-48 | 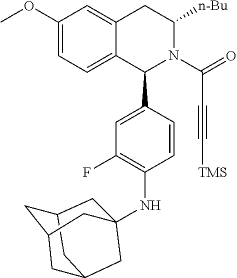 |
| A-55 | 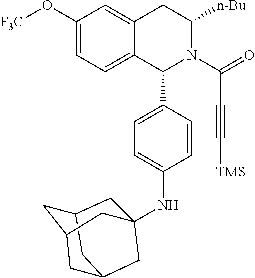 |
| A-57 | 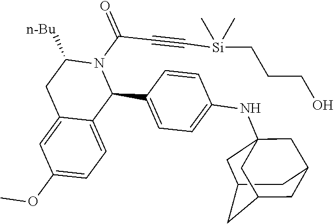 |

### Embodiment B

In certain embodiments, provided herein is a compound or a tautomer, stereoisomer, mixture of stereoisomers, isotopically enriched analog, or pharmaceutically acceptable salt thereof, represented by Formula B-I, as in Table B-1:

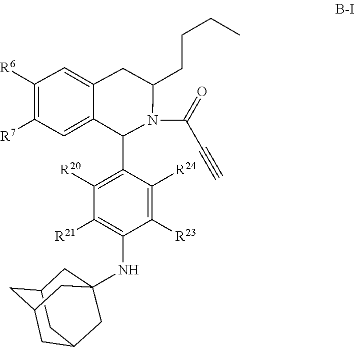

TABLE B-1
Selected compounds of the disclosure.

| Compound | | | | | | |
|---|---|---|---|---|---|---|
| No. | R⁶ | R⁷ | R²⁰ | R²¹ | R²³ | R²⁴ |
| B-I-1 | —OCH₃ | —H | —CN | —H | —H | —H |
| B-I-2 | —OCF₃ | —H | —CN | —H | —H | —H |
| B-I-3 | —CF₃ | —H | —CN | —H | —H | —H |
| B-I-4 | —CN | —H | —CN | —H | —H | —H |
| B-I-5 | —F | —H | —CN | —H | —H | —H |
| B-I-6 | —CF₂H | —H | —CN | —H | —H | —H |
| B-I-7 | —SO₂CH₃ | —H | —CN | —H | —H | —H |
| B-I-8 | —F | —F | —CN | —H | —H | —H |
| B-I-9 | —CN | —F | —CN | —H | —H | —H |
| B-I-10 | —F | —CN | —CN | —H | —H | —H |
| B-I-11 | —CF₃ | —F | —CN | —H | —H | —H |
| B-I-12 | —F | —CF₃ | —CN | —H | —H | —H |
| B-I-13 | —OCF₃ | —F | —CN | —H | —H | —H |
| B-I-14 | —F | —OCF₃ | —CN | —H | —H | —H |
| B-I-15 | —OCH₃ | —H | —CN | —F | —H | —H |
| B-I-16 | —OCF₃ | —H | —CN | —F | —H | —H |
| B-I-17 | —CF₃ | —H | —CN | —F | —H | —H |
| B-I-18 | —CN | —H | —CN | —F | —H | —H |
| B-I-19 | —F | —H | —CN | —F | —H | —H |
| B-I-20 | —CF₂H | —H | —CN | —F | —H | —H |
| B-I-21 | —SO₂CH₃ | —H | —CN | —F | —H | —H |
| B-I-22 | —F | —F | —CN | —F | —H | —H |
| B-I-23 | —CN | —F | —CN | —F | —H | —H |
| B-I-24 | —F | —CN | —CN | —F | —H | —H |
| B-I-25 | —CF₃ | —F | —CN | —F | —H | —H |
| B-I-26 | —F | —CF₃ | —CN | —F | —H | —H |
| B-I-27 | —OCF₃ | —F | —CN | —F | —H | —H |
| B-I-28 | —F | —OCF₃ | —CN | —F | —H | —H |
| B-I-29 | —OCH₃ | —H | —CN | —H | —F | —H |
| B-I-30 | —OCF₃ | —H | —CN | —H | —F | —H |
| B-I-31 | —CF₃ | —H | —CN | —H | —F | —H |
| B-I-32 | —CN | —H | —CN | —H | —F | —H |
| B-I-33 | —F | —H | —CN | —H | —F | —H |
| B-I-34 | —CF₂H | —H | —CN | —H | —F | —H |
| B-I-35 | —SO₂CH₃ | —H | —CN | —H | —F | —H |
| B-I-36 | —F | —F | —CN | —H | —F | —H |
| B-I-37 | —CN | —F | —CN | —H | —F | —H |
| B-I-38 | —F | —CN | —CN | —H | —F | —H |
| B-I-39 | —CF₃ | —F | —CN | —H | —F | —H |
| B-I-40 | —F | —CF₃ | —CN | —H | —F | —H |
| B-I-41 | —OCF₃ | —F | —CN | —H | —F | —H |
| B-I-42 | —F | —OCF₃ | —CN | —H | —F | —H |
| B-I-43 | —OCH₃ | —H | —CN | —H | —H | —F |
| B-I-44 | —OCF₃ | —H | —CN | —H | —H | —F |
| B-I-45 | —CF₃ | —H | —CN | —H | —H | —F |
| B-I-46 | —CN | —H | —CN | —H | —H | —F |
| B-I-47 | —F | —H | —CN | —H | —H | —F |
| B-I-48 | —CF₂H | —H | —CN | —H | —H | —F |
| B-I-49 | —SO₂CH₃ | —H | —CN | —H | —H | —F |
| B-I-50 | —F | —F | —CN | —H | —H | —F |
| B-I-51 | —CN | —F | —CN | —H | —H | —F |
| B-I-52 | —F | —CN | —CN | —H | —H | —F |
| B-I-53 | —CF₃ | —F | —CN | —H | —H | —F |
| B-I-54 | —F | —CF₃ | —CN | —H | —H | —F |
| B-I-55 | —OCF₃ | —F | —CN | —H | —H | —F |
| B-I-56 | —F | —OCF₃ | —CN | —H | —H | —F |
| B-I-57 | —OCH₃ | —H | —H | —CN | —H | —H |
| B-I-58 | —OCF₃ | —H | —H | —CN | —H | —H |
| B-I-59 | —CF₃ | —H | —H | —CN | —H | —H |
| B-I-60 | —CN | —H | —H | —CN | —H | —H |
| B-I-61 | —F | —H | —H | —CN | —H | —H |
| B-I-62 | —CF₂H | —H | —H | —CN | —H | —H |
| B-I-63 | —SO₂CH₃ | —H | —H | —CN | —H | —H |
| B-I-64 | —F | —F | —H | —CN | —H | —H |
| B-I-65 | —CN | —F | —H | —CN | —H | —H |
| B-I-66 | —F | —CN | —H | —CN | —H | —H |
| B-I-67 | —CF₃ | —F | —H | —CN | —H | —H |
| B-I-68 | —F | —CF₃ | —H | —CN | —H | —H |
| B-I-69 | —OCF₃ | —F | —H | —CN | —H | —H |
| B-I-70 | —F | —OCF₃ | —H | —CN | —H | —H |
| B-I-71 | —OCH₃ | —H | —F | —CN | —H | —H |
| B-I-72 | —OCF₃ | —H | —F | —CN | —H | —H |
| B-I-73 | —CF₃ | —H | —F | —CN | —H | —H |
| B-I-74 | —CN | —H | —F | —CN | —H | —H |
| B-I-75 | —F | —H | —F | —CN | —H | —H |
| B-I-76 | —CF₂H | —H | —F | —CN | —H | —H |
| B-I-77 | —SO₂CH₃ | —H | —F | —CN | —H | —H |
| B-I-78 | —F | —F | —F | —CN | —H | —H |
| B-I-79 | —CN | —F | —F | —CN | —H | —H |
| B-I-80 | —F | —CN | —F | —CN | —H | —H |
| B-I-81 | —CF₃ | —F | —F | —CN | —H | —H |
| B-I-82 | —F | —CF₃ | —F | —CN | —H | —H |
| B-I-83 | —OCF₃ | —F | —F | —CN | —H | —H |
| B-I-84 | —F | —OCF₃ | —F | —CN | —H | —H |
| B-I-85 | —OCH₃ | —H | —H | —CN | —F | —H |
| B-I-86 | —OCF₃ | —H | —H | —CN | —F | —H |
| B-I-87 | —CF₃ | —H | —H | —CN | —F | —H |
| B-I-88 | —CN | —H | —H | —CN | —F | —H |
| B-I-89 | —F | —H | —H | —CN | —F | —H |
| B-I-90 | —CF₂H | —H | —H | —CN | —F | —H |
| B-I-91 | —SO₂CH₃ | —H | —H | —CN | —F | —H |
| B-I-92 | —F | —F | —H | —CN | —F | —H |
| B-I-93 | —CN | —F | —H | —CN | —F | —H |
| B-I-94 | —F | —CN | —H | —CN | —F | —H |
| B-I-95 | —CF₃ | —F | —H | —CN | —F | —H |
| B-I-96 | —F | —CF₃ | —H | —CN | —F | —H |
| B-I-97 | —OCF₃ | —F | —H | —CN | —F | —H |
| B-I-98 | —F | —OCF₃ | —H | —CN | —F | —H |
| B-I-99 | —OCH₃ | —H | —H | —CN | —H | —F |
| B-I-100 | —OCF₃ | —H | —H | —CN | —H | —F |
| B-I-101 | —CF₃ | —H | —H | —CN | —H | —F |
| B-I-102 | —CN | —H | —H | —CN | —H | —F |
| B-I-103 | —F | —H | —H | —CN | —H | —F |
| B-I-104 | —CF₂H | —H | —H | —CN | —H | —F |
| B-I-105 | —SO₂CH₃ | —H | —H | —CN | —H | —F |
| B-I-106 | —F | —F | —H | —CN | —H | —F |
| B-I-107 | —CN | —F | —H | —CN | —H | —F |
| B-I-108 | —F | —CN | —H | —CN | —H | —F |
| B-I-109 | —CF₃ | —F | —H | —CN | —H | —F |
| B-I-110 | —F | —CF₃ | —H | —CN | —H | —F |
| B-I-111 | —OCF₃ | —F | —H | —CN | —H | —F |
| B-I-112 | —F | —OCF₃ | —H | —CN | —H | —F |
| B-I-113 | —OCH₃ | —H | —F | —H | —H | —H |
| B-I-114 | —OCF₃ | —H | —F | —H | —H | —H |
| B-I-115 | —CF₃ | —H | —F | —H | —H | —H |
| B-I-116 | —CN | —H | —F | —H | —H | —H |
| B-I-117 | —F | —H | —F | —H | —H | —H |
| B-I-118 | —CF₂H | —H | —F | —H | —H | —H |
| B-I-119 | —SO₂CH₃ | —H | —F | —H | —H | —H |
| B-I-120 | —F | —F | —F | —H | —H | —H |
| B-I-121 | —CN | —F | —F | —H | —H | —H |
| B-I-122 | —F | —CN | —F | —H | —H | —H |
| B-I-123 | —CF₃ | —F | —F | —H | —H | —H |
| B-I-124 | —F | —CF₃ | —F | —H | —H | —H |
| B-I-125 | —OCF₃ | —F | —F | —H | —H | —H |
| B-I-126 | —F | —OCF₃ | —F | —H | —H | —H |
| B-I-127 | —OCH₃ | —H | —H | —F | —H | —H |
| B-I-128 | —OCF₃ | —H | —H | —F | —H | —H |
| B-I-129 | —CF₃ | —H | —H | —F | —H | —H |
| B-I-130 | —CN | —H | —H | —F | —H | —H |
| B-I-131 | —F | —H | —H | —F | —H | —H |
| B-I-132 | —CF₂H | —H | —H | —F | —H | —H |
| B-I-133 | —SO₂CH₃ | —H | —H | —F | —H | —H |
| B-I-134 | —F | —F | —H | —F | —H | —H |
| B-I-135 | —CN | —F | —H | —F | —H | —H |
| B-I-136 | —F | —CN | —H | —F | —H | —H |
| B-I-137 | —CF₃ | —F | —H | —F | —H | —H |
| B-I-138 | —F | —CF₃ | —H | —F | —H | —H |
| B-I-139 | —OCF₃ | —F | —H | —F | —H | —H |
| B-I-140 | —F | —OCF₃ | —H | —F | —H | —H |
| B-I-141 | —OCH₃ | —H | —F | —F | —H | —H |
| B-I-142 | —OCF₃ | —H | —F | —F | —H | —H |
| B-I-143 | —CF₃ | —H | —F | —F | —H | —H |
| B-I-144 | —CN | —H | —F | —F | —H | —H |
| B-I-145 | —F | —H | —F | —F | —H | —H |
| B-I-146 | —CF₂H | —H | —F | —F | —H | —H |
| B-I-147 | —SO₂CH₃ | —H | —F | —F | —H | —H |
| B-I-148 | —F | —F | —F | —F | —H | —H |
| B-I-149 | —CN | —F | —F | —F | —H | —H |
| B-I-150 | —F | —CN | —F | —F | —H | —H |
| B-I-151 | —CF₃ | —F | —F | —F | —H | —H |
| B-I-152 | —F | —CF₃ | —F | —F | —H | —H |
| B-I-153 | —OCF₃ | —F | —F | —F | —H | —H |
| B-I-154 | —F | —OCF₃ | —F | —F | —H | —H |
| B-I-155 | —OCH₃ | —H | —F | —H | —F | —H |
| B-I-156 | —OCF₃ | —H | —F | —H | —F | —H |
| B-I-157 | —CF₃ | —H | —F | —H | —F | —H |
| B-I-158 | —CN | —H | —F | —H | —F | —H |
| B-I-159 | —F | —H | —F | —H | —F | —H |
| B-I-160 | —CF₂H | —H | —F | —H | —F | —H |
| B-I-161 | —SO₂CH₃ | —H | —F | —H | —F | —H |
| B-I-162 | —F | —F | —F | —H | —F | —H |
| B-I-163 | —CN | —F | —F | —H | —F | —H |
| B-I-164 | —F | —CN | —F | —H | —F | —H |
| B-I-165 | —CF₃ | —F | —F | —H | —F | —H |
| B-I-166 | —F | —CF₃ | —F | —H | —F | —H |
| B-I-167 | —OCF₃ | —F | —F | —H | —F | —H |
| B-I-168 | —F | —OCF₃ | —F | —H | —F | —H |
| B-I-169 | —OCH₃ | —H | —F | —H | —H | —F |
| B-I-170 | —OCF₃ | —H | —F | —H | —H | —F |
| B-I-171 | —CF₃ | —H | —F | —H | —H | —F |
| B-I-172 | —CN | —H | —F | —H | —H | —F |
| B-I-173 | —F | —H | —F | —H | —H | —F |
| B-I-174 | —CF₂H | —H | —F | —H | —H | —F |
| B-I-175 | —SO₂CH₃ | —H | —F | —H | —H | —F |
| B-I-176 | —F | —F | —F | —H | —H | —F |
| B-I-177 | —CN | —F | —F | —H | —H | —F |
| B-I-178 | —F | —CN | —F | —H | —H | —F |
| B-I-179 | —CF₃ | —F | —F | —H | —H | —F |
| B-I-180 | —F | —CF₃ | —F | —H | —H | —F |
| B-I-181 | —OCF₃ | —F | —F | —H | —H | —F |
| B-I-182 | —F | —OCF₃ | —F | —H | —H | —F |
| B-I-183 | —OCH₃ | —H | —H | —F | —F | —H |
| B-I-184 | —OCF₃ | —H | —H | —F | —F | —H |
| B-I-185 | —CF₃ | —H | —H | —F | —F | —H |
| B-I-186 | —CN | —H | —H | —F | —F | —H |
| B-I-187 | —F | —H | —H | —F | —F | —H |
| B-I-188 | —CF₂H | —H | —H | —F | —F | —H |
| B-I-189 | —SO₂CH₃ | —H | —H | —F | —F | —H |
| B-I-190 | —F | —F | —H | —F | —F | —H |
| B-I-191 | —CN | —F | —H | —F | —F | —H |
| B-I-192 | —F | —CN | —H | —F | —F | —H |
| B-I-193 | —CF₃ | —F | —H | —F | —F | —H |
| B-I-194 | —F | —CF₃ | —H | —F | —F | —H |
| B-I-195 | —OCF₃ | —F | —H | —F | —F | —H |
| B-I-196 | —F | —OCF₃ | —H | —F | —F | —H |
| B-I-197 | —OCH₃ | —H | —CF₃ | —H | —H | —H |
| B-I-198 | —OCF₃ | —H | —CF₃ | —H | —H | —H |
| B-I-199 | —CF₃ | —H | —CF₃ | —H | —H | —H |
| B-I-200 | —CN | —H | —CF₃ | —H | —H | —H |
| B-I-201 | —F | —H | —CF₃ | —H | —H | —H |
| B-I-202 | —CF₂H | —H | —CF₃ | —H | —H | —H |
| B-I-203 | —SO₂CH₃ | —H | —CF₃ | —H | —H | —H |
| B-I-204 | —F | —F | —CF₃ | —H | —H | —H |
| B-I-205 | —CN | —F | —CF₃ | —H | —H | —H |
| B-I-206 | —F | —CN | —CF₃ | —H | —H | —H |
| B-I-207 | —CF₃ | —F | —CF₃ | —H | —H | —H |
| B-I-208 | —F | —CF₃ | —CF₃ | —H | —H | —H |
| B-I-209 | —OCF₃ | —F | —CF₃ | —H | —H | —H |
| B-I-210 | —F | —OCF₃ | —CF₃ | —H | —H | —H |
| B-I-211 | —OCH₃ | —H | —CF₃ | —F | —H | —H |
| B-I-212 | —OCF₃ | —H | —CF₃ | —F | —H | —H |
| B-I-213 | —CF₃ | —H | —CF₃ | —F | —H | —H |
| B-I-214 | —CN | —H | —CF₃ | —F | —H | —H |
| B-I-215 | —F | —H | —CF₃ | —F | —H | —H |
| B-I-216 | —CF₂H | —H | —CF₃ | —F | —H | —H |
| B-I-217 | —SO₂CH₃ | —H | —CF₃ | —F | —H | —H |
| B-I-218 | —F | —F | —CF₃ | —F | —H | —H |
| B-I-219 | —CN | —F | —CF₃ | —F | —H | —H |
| B-I-220 | —F | —CN | —CF₃ | —F | —H | —H |
| B-I-221 | —CF₃ | —F | —CF₃ | —F | —H | —H |
| B-I-222 | —F | —CF₃ | —CF₃ | —F | —H | —H |
| B-I-223 | —OCF₃ | —F | —CF₃ | —F | —H | —H |
| B-I-224 | —F | —OCF₃ | —CF₃ | —F | —H | —H |
| B-I-225 | —OCH₃ | —H | —CF₃ | —H | —F | —H |
| B-I-226 | —OCF₃ | —H | —CF₃ | —H | —F | —H |
| B-I-227 | —CF₃ | —H | —CF₃ | —H | —F | —H |
| B-I-228 | —CN | —H | —CF₃ | —H | —F | —H |
| B-I-229 | —F | —H | —CF₃ | —H | —F | —H |
| B-I-230 | —CF₂H | —H | —CF₃ | —H | —F | —H |
| B-I-231 | —SO₂CH₃ | —H | —CF₃ | —H | —F | —H |
| B-I-232 | —F | —F | —CF₃ | —H | —F | —H |
| B-I-233 | —CN | —F | —CF₃ | —H | —F | —H |
| B-I-234 | —F | —CN | —CF₃ | —H | —F | —H |
| B-I-235 | —CF₃ | —F | —CF₃ | —H | —F | —H |
| B-I-236 | —F | —CF₃ | —CF₃ | —H | —F | —H |
| B-I-237 | —OCF₃ | —F | —CF₃ | —H | —F | —H |
| B-I-238 | —F | —OCF₃ | —CF₃ | —H | —F | —H |
| B-I-239 | —OCH₃ | —H | —CF₃ | —H | —H | —F |
| B-I-240 | —OCF₃ | —H | —CF₃ | —H | —H | —F |
| B-I-241 | —CF₃ | —H | —CF₃ | —H | —H | —F |
| B-I-242 | —CN | —H | —CF₃ | —H | —H | —F |
| B-I-243 | —F | —H | —CF₃ | —H | —H | —F |
| B-I-244 | —CF₂H | —H | —CF₃ | —H | —H | —F |
| B-I-245 | —SO₂CH₃ | —H | —CF₃ | —H | —H | —F |
| B-I-246 | —F | —F | —CF₃ | —H | —H | —F |
| B-I-247 | —CN | —F | —CF₃ | —H | —H | —F |
| B-I-248 | —F | —CN | —CF₃ | —H | —H | —F |
| B-I-249 | —CF₃ | —F | —CF₃ | —H | —H | —F |
| B-I-250 | —F | —CF₃ | —CF₃ | —H | —H | —F |
| B-I-251 | —OCF₃ | —F | —CF₃ | —H | —H | —F |
| B-I-252 | —F | —OCF₃ | —CF₃ | —H | —H | —F |
| B-I-253 | —OCH₃ | —H | —F | —CF₃ | —H | —H |
| B-I-254 | —OCF₃ | —H | —F | —CF₃ | —H | —H |
| B-I-255 | —CF₃ | —H | —F | —CF₃ | —H | —H |
| B-I-256 | —CN | —H | —F | —CF₃ | —H | —H |
| B-I-257 | —F | —H | —F | —CF₃ | —H | —H |
| B-I-258 | —CF₂H | —H | —F | —CF₃ | —H | —H |
| B-I-259 | —SO₂CH₃ | —H | —F | —CF₃ | —H | —H |
| B-I-260 | —F | —F | —F | —CF₃ | —H | —H |
| B-I-261 | —CN | —F | —F | —CF₃ | —H | —H |
| B-I-262 | —F | —CN | —F | —CF₃ | —H | —H |
| B-I-263 | —CF₃ | —F | —F | —CF₃ | —H | —H |
| B-I-264 | —F | —CF₃ | —F | —CF₃ | —H | —H |
| B-I-265 | —OCF₃ | —F | —F | —CF₃ | —H | —H |
| B-I-266 | —F | —OCF₃ | —F | —CF₃ | —H | —H |
| B-I-267 | —OCH₃ | —H | —H | —CF₃ | —F | —H |
| B-I-268 | —OCF₃ | —H | —H | —CF₃ | —F | —H |
| B-I-269 | —CF₃ | —H | —H | —CF₃ | —F | —H |
| B-I-270 | —CN | —H | —H | —CF₃ | —F | —H |
| B-I-271 | —F | —H | —H | —CF₃ | —F | —H |
| B-I-272 | —CF₂H | —H | —H | —CF₃ | —F | —H |
| B-I-273 | —SO₂CH₃ | —H | —H | —CF₃ | —F | —H |
| B-I-274 | —F | —F | —H | —CF₃ | —F | —H |
| B-I-275 | —CN | —F | —H | —CF₃ | —F | —H |
| B-I-276 | —F | —CN | —H | —CF₃ | —F | —H |
| B-I-277 | —CF₃ | —F | —H | —CF₃ | —F | —H |
| B-I-278 | —F | —CF₃ | —H | —CF₃ | —F | —H |
| B-I-279 | —OCF₃ | —F | —H | —CF₃ | —F | —H |
| B-I-280 | —F | —OCF₃ | —H | —CF₃ | —F | —H |
| B-I-281 | —OCH₃ | —H | —H | —CF₃ | —H | —F |
| B-I-282 | —OCF₃ | —H | —H | —CF₃ | —H | —F |
| B-I-283 | —CF₃ | —H | —H | —CF₃ | —H | —F |
| B-I-284 | —CN | —H | —H | —CF₃ | —H | —F |
| B-I-285 | —F | —H | —H | —CF₃ | —H | —F |
| B-I-286 | —CF₂H | —H | —H | —CF₃ | —H | —F |
| B-I-287 | —SO₂CH₃ | —H | —H | —CF₃ | —H | —F |
| B-I-288 | —F | —F | —H | —CF₃ | —H | —F |
| B-I-289 | —CN | —F | —H | —CF₃ | —H | —F |
| B-I-290 | —F | —CN | —H | —CF₃ | —H | —F |
| B-I-291 | —CF₃ | —F | —H | —CF₃ | —H | —F |
| B-I-292 | —F | —CF₃ | —H | —CF₃ | —H | —F |
| B-I-293 | —OCF₃ | —F | —H | —CF₃ | —H | —F |
| B-I-294 | —F | —OCF₃ | —H | —CF₃ | —H | —F |

In certain embodiments, provided herein is a compound, or a tautomer, stereoisomer, mixture of stereoisomers, isotopically enriched analog, or pharmaceutically acceptable salt thereof, represented by Formula B-IA, as in Table B-2:

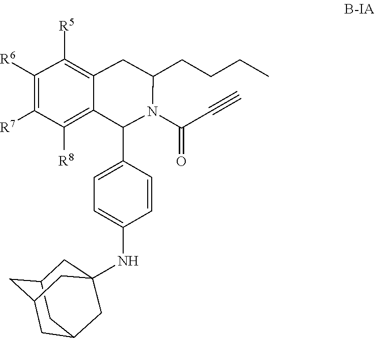

TABLE B-2
Selected compounds of the disclosure.

| Compound No. | R⁵ | R⁶ | R⁷ | R⁸ |
|---|---|---|---|---|
| B-IA-1 | —OCH₃ | —H | —H | —H |
| B-IA-2 | —H | —OCH₃ | —H | —H |
| B-IA-3 | —H | —H | —OCH₃ | —H |
| B-IA-4 | —H | —H | —H | —OCH₃ |
| B-IA-5 | —OCF₃ | —H | —H | —H |
| B-IA-6 | —H | —OCF₃ | —H | —H |
| B-IA-7 | —H | —H | —OCF₃ | —H |
| B-IA-8 | —H | —H | —H | —OCF₃ |
| B-IA-9 | —CF₃ | —H | —H | —H |
| B-IA-10 | —H | —CF₃ | —H | —H |
| B-IA-11 | —H | —H | —CF₃ | —H |
| B-IA-12 | —H | —H | —H | —CF₃ |
| B-IA-13 | —CN | —H | —H | —H |
| B-IA-14 | —H | —CN | —H | —H |
| B-IA-15 | —H | —H | —CN | —H |
| B-IA-16 | —H | —H | —H | —CN |
| B-IA-17 | —F | —H | —H | —H |
| B-IA-18 | —H | —F | —H | —H |
| B-IA-19 | —H | —H | —F | —H |
| B-IA-20 | —H | —H | —H | —F |
| B-IA-21 | —CF₂H | —H | —H | —H |
| B-IA-22 | —H | —CF₂H | —H | —H |
| B-IA-23 | —H | —H | —CF₂H | —H |
| B-IA-24 | —H | —H | —H | —CF₂H |
| B-IA-25 | —SO₂CH₃ | —H | —H | —H |
| B-IA-26 | —H | —SO₂CH₃ | —H | —H |
| B-IA-27 | —H | —H | —SO₂CH₃ | —H |
| B-IA-28 | —H | —H | —H | —SO₂CH₃ |
| B-IA-29 | —SO₂NHCH₃ | —H | —H | —H |
| B-IA-30 | —H | —SO₂NHCH₃ | —H | —H |
| B-IA-31 | —H | —H | —SO₂NHCH₃ | —H |
| B-IA-32 | —H | —H | —H | —SO₂NHCH₃ |

In certain embodiments, provided herein is a compound, or a tautomer, stereoisomer, mixture of stereoisomers, isotopically enriched analog, or pharmaceutically acceptable salt thereof, represented by Formula B-IB, as in Table B-3:

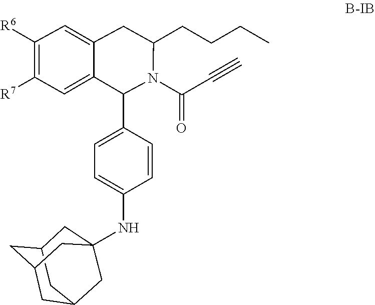

TABLE B-3
Selected compounds of the disclosure.

| Compound | | |
|---|---|---|
| No. | R⁶ | R⁷ |
| B-IB-1 | —OCH₃ | —F |
| B-IB-2 | —F | —OCH₃ |
| B-IB-3 | —OCF₃ | —F |
| B-IB-4 | —F | —OCF₃ |
| B-IB-5 | —F | —F |
| B-IB-6 | —OCH₃ | —CN |
| B-IB-7 | —CN | —OCH₃ |
| B-IB-8 | —SO₂CH₃ | —F |
| B-IB-9 | —F | —SO₂CH₃ |
| B-IB-10 | —CN | —F |
| B-IB-11 | —F | —CN |

In certain embodiments, provided herein is a compound, or a tautomer, stereoisomer, mixture of stereoisomers, isotopically enriched analog, or pharmaceutically acceptable salt thereof, represented by Formula B-II, as in Table B-4:

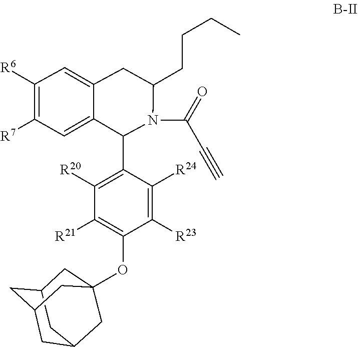

TABLE B-4

| Compound | | | | | | |
|---|---|---|---|---|---|---|
| No. | R⁶ | R⁷ | R²⁰ | R²¹ | R²³ | R²⁴ |
| B-II-1 | —OCH₃ | —H | —CN | —H | —H | —H |
| B-II-2 | —OCF₃ | —H | —CN | —H | —H | —H |
| B-II-3 | —CF₃ | —H | —CN | —H | —H | —H |
| B-II-4 | —CN | —H | —CN | —H | —H | —H |
| B-II-5 | —F | —H | —CN | —H | —H | —H |
| B-II-6 | —CF₂H | —H | —CN | —H | —H | —H |
| B-II-7 | —SO₂CH₃ | —H | —CN | —H | —H | —H |
| B-II-8 | —F | —F | —CN | —H | —H | —H |
| B-II-9 | —CN | —F | —CN | —H | —H | —H |
| B-II-10 | —F | —CN | —CN | —H | —H | —H |
| B-II-11 | —CF₃ | —F | —CN | —H | —H | —H |
| B-II-12 | —F | —CF₃ | —CN | —H | —H | —H |
| B-II-13 | —OCF₃ | —F | —CN | —H | —H | —H |
| B-II-14 | —F | —OCF₃ | —CN | —H | —H | —H |
| B-II-15 | —OCH₃ | —H | —CN | —F | —H | —H |
| B-II-16 | —OCF₃ | —H | —CN | —F | —H | —H |
| B-II-17 | —CF₃ | —H | —CN | —F | —H | —H |
| B-II-18 | —CN | —H | —CN | —F | —H | —H |
| B-II-19 | —F | —H | —CN | —F | —H | —H |
| B-II-20 | —CF₂H | —H | —CN | —F | —H | —H |
| B-II-21 | —SO₂CH₃ | —H | —CN | —F | —H | —H |
| B-II-22 | —F | —F | —CN | —F | —H | —H |
| B-II-23 | —CN | —F | —CN | —F | —H | —H |
| B-II-24 | —F | —CN | —CN | —F | —H | —H |
| B-II-25 | —CF₃ | —F | —CN | —F | —H | —H |
| B-II-26 | —F | —CF₃ | —CN | —F | —H | —H |
| B-II-27 | —OCF₃ | —F | —CN | —F | —H | —H |
| B-II-28 | —F | —OCF₃ | —CN | —F | —H | —H |
| B-II-29 | —OCH₃ | —H | —CN | —H | —F | —H |
| B-II-30 | —OCF₃ | —H | —CN | —H | —F | —H |
| B-II-31 | —CF₃ | —H | —CN | —H | —F | —H |
| B-II-32 | —CN | —H | —CN | —H | —F | —H |
| B-II-33 | —F | —H | —CN | —H | —F | —H |
| B-II-34 | —CF₂H | —H | —CN | —H | —F | —H |
| B-II-35 | —SO₂CH₃ | —H | —CN | —H | —F | —H |
| B-II-36 | —F | —F | —CN | —H | —F | —H |
| B-II-37 | —CN | —F | —CN | —H | —F | —H |
| B-II-38 | —F | —CN | —CN | —H | —F | —H |
| B-II-39 | —CF₃ | —F | —CN | —H | —F | —H |
| B-II-40 | —F | —CF₃ | —CN | —H | —F | —H |
| B-II-41 | —OCF₃ | —F | —CN | —H | —F | —H |
| B-II-42 | —F | —OCF₃ | —CN | —H | —F | —H |
| B-II-43 | —OCH₃ | —H | —CN | —H | —H | —F |
| B-II-44 | —OCF₃ | —H | —CN | —H | —H | —F |
| B-II-45 | —CF₃ | —H | —CN | —H | —H | —F |
| B-II-46 | —CN | —H | —CN | —H | —H | —F |
| B-II-47 | —F | —H | —CN | —H | —H | —F |
| B-II-48 | —CF₂H | —H | —CN | —H | —H | —F |
| B-II-49 | —SO₂CH₃ | —H | —CN | —H | —H | —F |
| B-II-50 | —F | —F | —CN | —H | —H | —F |
| B-II-51 | —CN | —F | —CN | —H | —H | —F |
| B-II-52 | —F | —CN | —CN | —H | —H | —F |
| B-II-53 | —CF₃ | —F | —CN | —H | —H | —F |
| B-II-54 | —F | —CF₃ | —CN | —H | —H | —F |
| B-II-55 | —OCF₃ | —F | —CN | —H | —H | —F |
| B-II-56 | —F | —OCF₃ | —CN | —H | —H | —F |
| B-II-57 | —OCH₃ | —H | —H | —CN | —H | —H |
| B-II-58 | —OCF₃ | —H | —H | —CN | —H | —H |
| B-II-59 | —CF₃ | —H | —H | —CN | —H | —H |
| B-II-60 | —CN | —H | —H | —CN | —H | —H |
| B-II-61 | —F | —H | —H | —CN | —H | —H |
| B-II-62 | —CF₂H | —H | —H | —CN | —H | —H |
| B-II-63 | —SO₂CH₃ | —H | —H | —CN | —H | —H |
| B-II-64 | —F | —F | —H | —CN | —H | —H |
| B-II-65 | —CN | —F | —H | —CN | —H | —H |
| B-II-66 | —F | —CN | —H | —CN | —H | —H |
| B-II-67 | —CF₃ | —F | —H | —CN | —H | —H |
| B-II-68 | —F | —CF₃ | —H | —CN | —H | —H |
| B-II-69 | —OCF₃ | —F | —H | —CN | —H | —H |
| B-II-70 | —F | —OCF₃ | —H | —CN | —H | —H |
| B-II-71 | —OCH₃ | —H | —F | —CN | —H | —H |
| B-II-72 | —OCF₃ | —H | —F | —CN | —H | —H |
| B-II-73 | —CF₃ | —H | —F | —CN | —H | —H |
| B-II-74 | —CN | —H | —F | —CN | —H | —H |
| B-II-75 | —F | —H | —F | —CN | —H | —H |
| B-II-76 | —CF₂H | —H | —F | —CN | —H | —H |
| B-II-77 | —SO₂CH₃ | —H | —F | —CN | —H | —H |
| B-II-78 | —F | —F | —F | —CN | —H | —H |
| B-II-79 | —CN | —F | —F | —CN | —H | —H |
| B-II-80 | —F | —CN | —F | —CN | —H | —H |
| B-II-81 | —CF₃ | —F | —F | —CN | —H | —H |
| B-II-82 | —F | —CF₃ | —F | —CN | —H | —H |
| B-II-83 | —OCF₃ | —F | —F | —CN | —H | —H |
| B-II-84 | —F | —OCF₃ | —F | —CN | —H | —H |
| B-II-85 | —OCH₃ | —H | —H | —CN | —F | —H |
| B-II-86 | —OCF₃ | —H | —H | —CN | —F | —H |
| B-II-87 | —CF₃ | —H | —H | —CN | —F | —H |
| B-II-88 | —CN | —H | —H | —CN | —F | —H |
| B-II-89 | —F | —H | —H | —CN | —F | —H |
| B-II-90 | —CF₂H | —H | —H | —CN | —F | —H |
| B-II-91 | —SO₂CH₃ | —H | —H | —CN | —F | —H |
| B-II-92 | —F | —F | —H | —CN | —F | —H |
| B-II-93 | —CN | —F | —H | —CN | —F | —H |
| B-II-94 | —F | —CN | —H | —CN | —F | —H |
| B-II-95 | —CF₃ | —F | —H | —CN | —F | —H |
| B-II-96 | —F | —CF₃ | —H | —CN | —F | —H |
| B-II-97 | —OCF₃ | —F | —H | —CN | —F | —H |
| B-II-98 | —F | —OCF₃ | —H | —CN | —F | —H |
| B-II-99 | —OCH₃ | —H | —H | —CN | —H | —F |
| B-II-100 | —OCF₃ | —H | —H | —CN | —H | —F |
| B-II-101 | —CF₃ | —H | —H | —CN | —H | —F |
| B-II-102 | —CN | —H | —H | —CN | —H | —F |
| B-II-103 | —F | —H | —H | —CN | —H | —F |
| B-II-104 | —CF₂H | —H | —H | —CN | —H | —F |
| B-II-105 | —SO₂CH₃ | —H | —H | —CN | —H | —F |
| B-II-106 | —F | —F | —H | —CN | —H | —F |
| B-II-107 | —CN | —F | —H | —CN | —H | —F |
| B-II-108 | —F | —CN | —H | —CN | —H | —F |
| B-II-109 | —CF₃ | —F | —H | —CN | —H | —F |
| B-II-110 | —F | —CF₃ | —H | —CN | —H | —F |
| B-II-111 | —OCF₃ | —F | —H | —CN | —H | —F |
| B-II-112 | —F | —OCF₃ | —H | —CN | —H | —F |
| B-II-113 | —OCH₃ | —H | —F | —H | —H | —H |
| B-II-114 | —OCF₃ | —H | —F | —H | —H | —H |
| B-II-115 | —CF₃ | —H | —F | —H | —H | —H |
| B-II-116 | —CN | —H | —F | —H | —H | —H |
| B-II-117 | —F | —H | —F | —H | —H | —H |
| B-II-118 | —CF₂H | —H | —F | —H | —H | —H |
| B-II-119 | —SO₂CH₃ | —H | —F | —H | —H | —H |
| B-II-120 | —F | —F | —F | —H | —H | —H |
| B-II-121 | —CN | —F | —F | —H | —H | —H |
| B-II-122 | —F | —CN | —F | —H | —H | —H |
| B-II-123 | —CF₃ | —F | —F | —H | —H | —H |
| B-II-124 | —F | —CF₃ | —F | —H | —H | —H |
| B-II-125 | —OCF₃ | —F | —F | —H | —H | —H |
| B-II-126 | —F | —OCF₃ | —F | —H | —H | —H |
| B-II-127 | —OCH₃ | —H | —H | —F | —H | —H |
| B-II-128 | —OCF₃ | —H | —H | —F | —H | —H |
| B-II-129 | —CF₃ | —H | —H | —F | —H | —H |
| B-II-130 | —CN | —H | —H | —F | —H | —H |
| B-II-131 | —F | —H | —H | —F | —H | —H |
| B-II-132 | —CF₂H | —H | —H | —F | —H | —H |
| B-II-133 | —SO₂CH₃ | —H | —H | —F | —H | —H |
| B-II-134 | —F | —F | —H | —F | —H | —H |
| B-II-135 | —CN | —F | —H | —F | —H | —H |
| B-II-136 | —F | —CN | —H | —F | —H | —H |
| B-II-137 | —CF₃ | —F | —H | —F | —H | —H |
| B-II-138 | —F | —CF₃ | —H | —F | —H | —H |
| B-II-139 | —OCF₃ | —F | —H | —F | —H | —H |
| B-II-140 | —F | —OCF₃ | —H | —F | —H | —H |
| B-II-141 | —OCH₃ | —H | —F | —F | —H | —H |
| B-II-142 | —OCF₃ | —H | —F | —F | —H | —H |
| B-II-143 | —CF₃ | —H | —F | —F | —H | —H |
| B-II-144 | —CN | —H | —F | —F | —H | —H |
| B-II-145 | —F | —H | —F | —F | —H | —H |
| B-II-146 | —CF₂H | —H | —F | —F | —H | —H |
| B-II-147 | —SO₂CH₃ | —H | —F | —F | —H | —H |
| B-II-148 | —F | —F | —F | —F | —H | —H |
| B-II-149 | —CN | —F | —F | —F | —H | —H |
| B-II-150 | —F | —CN | —F | —F | —H | —H |
| B-II-151 | —CF₃ | —F | —F | —F | —H | —H |
| B-II-152 | —F | —CF₃ | —F | —F | —H | —H |
| B-II-153 | —OCF₃ | —F | —F | —F | —H | —H |
| B-II-154 | —F | —OCF₃ | —F | —F | —H | —H |
| B-II-155 | —OCH₃ | —H | —F | —H | —F | —H |
| B-II-156 | —OCF₃ | —H | —F | —H | —F | —H |
| B-II-157 | —CF₃ | —H | —F | —H | —F | —H |
| B-II-158 | —CN | —H | —F | —H | —F | —H |
| B-II-159 | —F | —H | —F | —H | —F | —H |
| B-II-160 | —CF₂H | —H | —F | —H | —F | —H |
| B-II-161 | —SO₂CH₃ | —H | —F | —H | —F | —H |
| B-II-162 | —F | —F | —F | —H | —F | —H |
| B-II-163 | —CN | —F | —F | —H | —F | —H |
| B-II-164 | —F | —CN | —F | —H | —F | —H |
| B-II-165 | —CF₃ | —F | —F | —H | —F | —H |
| B-II-166 | —F | —CF₃ | —F | —H | —F | —H |
| B-II-167 | —OCF₃ | —F | —F | —H | —F | —H |
| B-II-168 | —F | —OCF₃ | —F | —H | —F | —H |
| B-II-169 | —OCH₃ | —H | —F | —H | —H | —F |
| B-II-170 | —OCF₃ | —H | —F | —H | —H | —F |
| B-II-171 | —CF₃ | —H | —F | —H | —H | —F |
| B-II-172 | —CN | —H | —F | —H | —H | —F |
| B-II-173 | —F | —H | —F | —H | —H | —F |
| B-II-174 | —CF₂H | —H | —F | —H | —H | —F |
| B-II-175 | —SO₂CH₃ | —H | —F | —H | —H | —F |
| B-II-176 | —F | —F | —F | —H | —H | —F |
| B-II-177 | —CN | —F | —F | —H | —H | —F |
| B-II-178 | —F | —CN | —F | —H | —H | —F |
| B-II-179 | —CF₃ | —F | —F | —H | —H | —F |
| B-II-180 | —F | —CF₃ | —F | —H | —H | —F |
| B-II-181 | —OCF₃ | —F | —F | —H | —H | —F |
| B-II-182 | —F | —OCF₃ | —F | —H | —H | —F |
| B-II-183 | —OCH₃ | —H | —H | —F | —F | —H |
| B-II-184 | —OCF₃ | —H | —H | —F | —F | —H |
| B-II-185 | —CF₃ | —H | —H | —F | —F | —H |
| B-II-186 | —CN | —H | —H | —F | —F | —H |
| B-II-187 | —F | —H | —H | —F | —F | —H |
| B-II-188 | —CF₂H | —H | —H | —F | —F | —H |
| B-II-189 | —SO₂CH₃ | —H | —H | —F | —F | —H |
| B-II-190 | —F | —F | —H | —F | —F | —H |
| B-II-191 | —CN | —F | —H | —F | —F | —H |
| B-II-192 | —F | —CN | —H | —F | —F | —H |
| B-II-193 | —CF₃ | —F | —H | —F | —F | —H |
| B-II-194 | —F | —CF₃ | —H | —F | —F | —H |
| B-II-195 | —OCF₃ | —F | —H | —F | —F | —H |
| B-II-196 | —F | —OCF₃ | —H | —F | —F | —H |
| B-II-197 | —OCH₃ | —H | —CF₃ | —H | —H | —H |
| B-II-198 | —OCF₃ | —H | —CF₃ | —H | —H | —H |
| B-II-199 | —CF₃ | —H | —CF₃ | —H | —H | —H |
| B-II-200 | —CN | —H | —CF₃ | —H | —H | —H |
| B-II-201 | —F | —H | —CF₃ | —H | —H | —H |
| B-II-202 | —CF₂H | —H | —CF₃ | —H | —H | —H |
| B-II-203 | —SO₂CH₃ | —H | —CF₃ | —H | —H | —H |
| B-II-204 | —F | —F | —CF₃ | —H | —H | —H |
| B-II-205 | —CN | —F | —CF₃ | —H | —H | —H |
| B-II-206 | —F | —CN | —CF₃ | —H | —H | —H |
| B-II-207 | —CF₃ | —F | —CF₃ | —H | —H | —H |
| B-II-208 | —F | —CF₃ | —CF₃ | —H | —H | —H |
| B-II-209 | —OCF₃ | —F | —CF₃ | —H | —H | —H |
| B-II-210 | —F | —OCF₃ | —CF₃ | —H | —H | —H |
| B-II-211 | —OCH₃ | —H | —CF₃ | —F | —H | —H |
| B-II-212 | —OCF₃ | —H | —CF₃ | —F | —H | —H |
| B-II-213 | —CF₃ | —H | —CF₃ | —F | —H | —H |
| B-II-214 | —CN | —H | —CF₃ | —F | —H | —H |
| B-II-215 | —F | —H | —CF₃ | —F | —H | —H |
| B-II-216 | —CF₂H | —H | —CF₃ | —F | —H | —H |
| B-II-217 | —SO₂CH₃ | —H | —CF₃ | —F | —H | —H |
| B-II-218 | —F | —F | —CF₃ | —F | —H | —H |
| B-II-219 | —CN | —F | —CF₃ | —F | —H | —H |
| B-II-220 | —F | —CN | —CF₃ | —F | —H | —H |
| B-II-221 | —CF₃ | —F | —CF₃ | —F | —H | —H |
| B-II-222 | —F | —CF₃ | —CF₃ | —F | —H | —H |
| B-II-223 | —OCF₃ | —F | —CF₃ | —F | —H | —H |
| B-II-224 | —F | —OCF₃ | —CF₃ | —F | —H | —H |
| B-II-225 | —OCH₃ | —H | —CF₃ | —H | —F | —H |
| B-II-226 | —OCF₃ | —H | —CF₃ | —H | —F | —H |
| B-II-227 | —CF₃ | —H | —CF₃ | —H | —F | —H |
| B-II-228 | —CN | —H | —CF₃ | —H | —F | —H |
| B-II-229 | —F | —H | —CF₃ | —H | —F | —H |
| B-II-230 | —CF₂H | —H | —CF₃ | —H | —F | —H |
| B-II-231 | —SO₂CH₃ | —H | —CF₃ | —H | —F | —H |
| B-II-232 | —F | —F | —CF₃ | —H | —F | —H |
| B-II-233 | —CN | —F | —CF₃ | —H | —F | —H |
| B-II-234 | —F | —CN | —CF₃ | —H | —F | —H |
| B-II-235 | —CF₃ | —F | —CF₃ | —H | —F | —H |
| B-II-236 | —F | —CF₃ | —CF₃ | —H | —F | —H |
| B-II-237 | —OCF₃ | —F | —CF₃ | —H | —F | —H |
| B-II-238 | —F | —OCF₃ | —CF₃ | —H | —F | —H |
| B-II-239 | —OCH₃ | —H | —CF₃ | —H | —H | —F |
| B-II-240 | —OCF₃ | —H | —CF₃ | —H | —H | —F |
| B-II-241 | —CF₃ | —H | —CF₃ | —H | —H | —F |
| B-II-242 | —CN | —H | —CF₃ | —H | —H | —F |
| B-II-243 | —F | —H | —CF₃ | —H | —H | —F |
| B-II-244 | —CF₂H | —H | —CF₃ | —H | —H | —F |
| B-II-245 | —SO₂CH₃ | —H | —CF₃ | —H | —H | —F |
| B-II-246 | —F | —F | —CF₃ | —H | —H | —F |
| B-II-247 | —CN | —F | —CF₃ | —H | —H | —F |
| B-II-248 | —F | —CN | —CF₃ | —H | —H | —F |
| B-II-249 | —CF₃ | —F | —CF₃ | —H | —H | —F |
| B-II-250 | —F | —CF₃ | —CF₃ | —H | —H | —F |
| B-II-251 | —OCF₃ | —F | —CF₃ | —H | —H | —F |
| B-II-252 | —F | —OCF₃ | —CF₃ | —H | —H | —F |
| B-II-253 | —OCH₃ | —H | —F | —CF₃ | —H | —H |
| B-II-254 | —OCF₃ | —H | —F | —CF₃ | —H | —H |
| B-II-255 | —CF₃ | —H | —F | —CF₃ | —H | —H |
| B-II-256 | —CN | —H | —F | —CF₃ | —H | —H |
| B-II-257 | —F | —H | —F | —CF₃ | —H | —H |
| B-II-258 | —CF₂H | —H | —F | —CF₃ | —H | —H |
| B-II-259 | —SO₂CH₃ | —H | —F | —CF₃ | —H | —H |
| B-II-260 | —F | —F | —F | —CF₃ | —H | —H |
| B-II-261 | —CN | —F | —F | —CF₃ | —H | —H |
| B-II-262 | —F | —CN | —F | —CF₃ | —H | —H |
| B-II-263 | —CF₃ | —F | —F | —CF₃ | —H | —H |
| B-II-264 | —F | —CF₃ | —F | —CF₃ | —H | —H |
| B-II-265 | —OCF₃ | —F | —F | —CF₃ | —H | —H |
| B-II-266 | —F | —OCF₃ | —F | —CF₃ | —H | —H |
| B-II-267 | —OCH₃ | —H | —H | —CF₃ | —F | —H |
| B-II-268 | —OCF₃ | —H | —H | —CF₃ | —F | —H |
| B-II-269 | —CF₃ | —H | —H | —CF₃ | —F | —H |
| B-II-270 | —CN | —H | —H | —CF₃ | —F | —H |
| B-II-271 | —F | —H | —H | —CF₃ | —F | —H |
| B-II-272 | —CF₂H | —H | —H | —CF₃ | —F | —H |
| B-II-273 | —SO₂CH₃ | —H | —H | —CF₃ | —F | —H |
| B-II-274 | —F | —F | —H | —CF₃ | —F | —H |
| B-II-275 | —CN | —F | —H | —CF₃ | —F | —H |
| B-II-276 | —F | —CN | —H | —CF₃ | —F | —H |
| B-II-277 | —CF₃ | —F | —H | —CF₃ | —F | —H |
| B-II-278 | —F | —CF₃ | —H | —CF₃ | —F | —H |
| B-II-279 | —OCF₃ | —F | —H | —CF₃ | —F | —H |
| B-II-280 | —F | —OCF₃ | —H | —CF₃ | —F | —H |
| B-II-281 | —OCH₃ | —H | —H | —CF₃ | —H | —F |
| B-II-282 | —OCF₃ | —H | —H | —CF₃ | —H | —F |
| B-II-283 | —CF₃ | —H | —H | —CF₃ | —H | —F |
| B-II-284 | —CN | —H | —H | —CF₃ | —H | —F |
| B-II-285 | —F | —H | —H | —CF₃ | —H | —F |
| B-II-286 | —CF₂H | —H | —H | —CF₃ | —H | —F |
| B-II-287 | —SO₂CH₃ | —H | —H | —CF₃ | —H | —F |
| B-II-288 | —F | —F | —H | —CF₃ | —H | —F |
| B-II-289 | —CN | —F | —H | —CF₃ | —H | —F |
| B-II-290 | —F | —CN | —H | —CF₃ | —H | —F |
| B-II-291 | —CF₃ | —F | —H | —CF₃ | —H | —F |
| B-II-292 | —F | —CF₃ | —H | —CF₃ | —H | —F |
| B-II-293 | —OCF₃ | —F | —H | —CF₃ | —H | —F |
| B-II-294 | —F | —OCF₃ | —H | —CF₃ | —H | —F |

In certain embodiments, provided herein is a compound, or a tautomer, stereoisomer, mixture of stereoisomers, isotopically enriched analog, or pharmaceutically acceptable salt thereof, represented by Formula B-IIA, as in Table B-5:

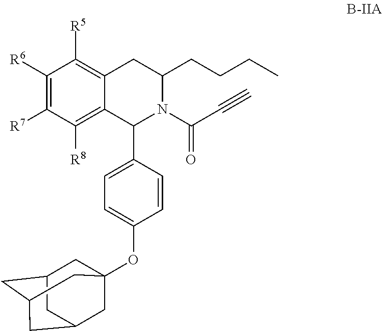

TABLE B-5

| Compound No. | R⁵ | R⁶ | R⁷ | R⁸ |
|---|---|---|---|---|
| B-IIA-1 | —OCH₃ | —H | —H | —H |
| B-IIA-2 | —H | —OCH₃ | —H | —H |
| B-IIA-3 | —H | —H | —OCH₃ | —H |
| B-IIA-4 | —H | —H | —H | —OCH₃ |
| B-IIA-5 | —OCF₃ | —H | —H | —H |
| B-IIA-6 | —H | —OCF₃ | —H | —H |
| B-IIA-7 | —H | —H | —OCF₃ | —H |
| B-IIA-8 | —H | —H | —H | —OCF₃ |
| B-IIA-9 | —CF₃ | —H | —H | —H |
| B-IIA-10 | —H | —CF₃ | —H | —H |
| B-IIA-11 | —H | —H | —CF₃ | —H |
| B-IIA-12 | —H | —H | —H | —CF₃ |
| B-IIA-13 | —CN | —H | —H | —H |
| B-IIA-14 | —H | —CN | —H | —H |
| B-IIA-15 | —H | —H | —CN | —H |
| B-IIA-16 | —H | —H | —H | —CN |
| B-IIA-17 | —F | —H | —H | —H |
| B-IIA-18 | —H | —F | —H | —H |
| B-IIA-19 | —H | —H | —F | —H |
| B-IIA-20 | —H | —H | —H | —F |
| B-IIA-21 | —CF₂H | —H | —H | —H |
| B-IIA-22 | —H | —CF₂H | —H | —H |
| B-IIA-23 | —H | —H | —CF₂H | —H |
| B-IIA-24 | —H | —H | —H | —CF₂H |
| B-IIA-25 | —SO₂CH₃ | —H | —H | —H |
| B-IIA-26 | —H | —SO₂CH₃ | —H | —H |
| B-IIA-27 | —H | —H | —SO₂CH₃ | —H |
| B-IIA-28 | —H | —H | —H | —SO₂CH₃ |
| B-IIA-29 | —SO₂NHCH₃ | —H | —H | —H |
| B-IIA-30 | —H | —SO₂NHCH₃ | —H | —H |
| B-IIA-31 | —H | —H | —SO₂NHCH₃ | —H |
| B-IIA-32 | —H | —H | —H | —SO₂NHCH₃ |

In certain embodiments, provided herein is a compound, or a tautomer, stereoisomer, mixture of stereoisomers, isotopically enriched analog, or pharmaceutically acceptable salt thereof, represented by Formula B-IIB, as in Table B-6:

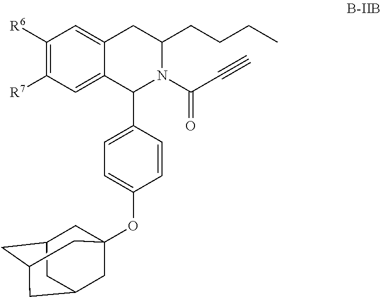

TABLE B-6

| Compound | | |
|---|---|---|
| No. | R⁶ | R⁷ |
| B-IIB-1 | —OCH₃ | —F |
| B-IIB-2 | —F | —OCH₃ |
| B-IIB-3 | —OCF₃ | —F |
| B-IIB-4 | —F | —OCF₃ |
| B-IIB-5 | —F | —F |
| B-IIB-6 | —OCH₃ | —CN |
| B-IIB-7 | —CN | —OCH₃ |
| B-IIB-8 | —SO₂CH₃ | —F |
| B-IIB-9 | —F | —SO₂CH₃ |
| B-IIB-10 | —CN | —F |
| B-IIB-11 | —F | —CN |

In certain embodiments, provided herein is a compound, or a tautomer, stereoisomer, mixture of stereoisomers, isotopically enriched analog, or pharmaceutically acceptable salt thereof, as in Table B-7:

TABLE B-7
Selected compounds of the disclosure.

| Compound No. | Structure |
|---|---|
| B-2 | 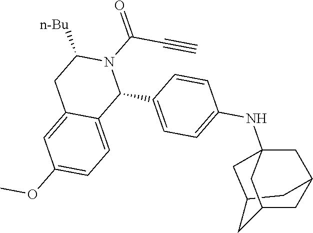 |
| B-3 | 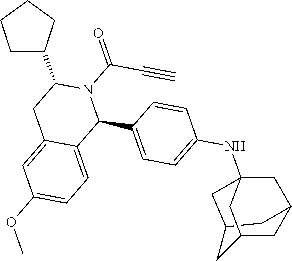 [SMILES: C#CC(=O)N1[C@@H](c2ccc(NC34CC5CC(CC(C5)C3)C4)cc2)c2ccc(OC)cc2C[C@@H]1C1CCCC1] |
| B-4 | 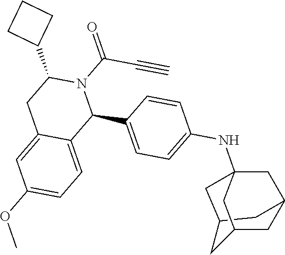 [SMILES: C#CC(=O)N1[C@@H](c2ccc(NC34CC5CC(CC(C5)C3)C4)cc2)c2ccc(OC)cc2C[C@@H]1C1CCC1] |
| B-5 | 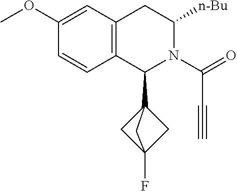 |
| B-6 | 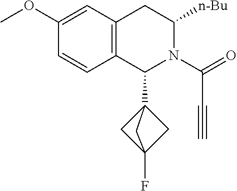 |
| B-7 | 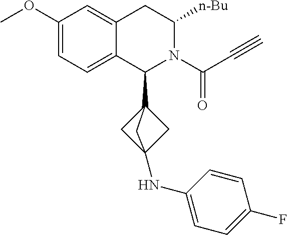 |
| B-8 | 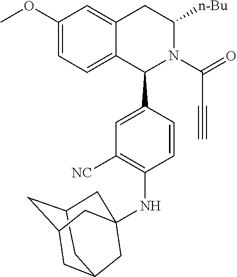 |
| B-9 | 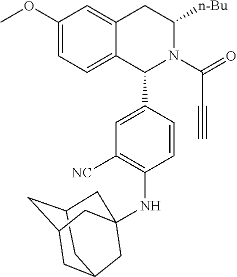 |
| B-10 | 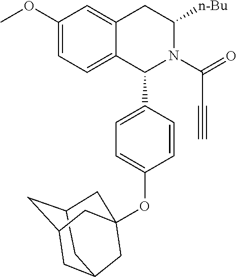 |
| B-11 | 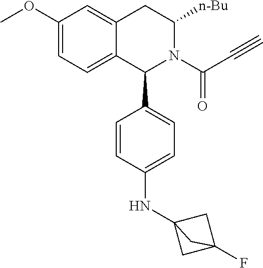 |
| B-12 | 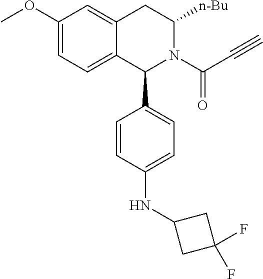 |
| B-13 | 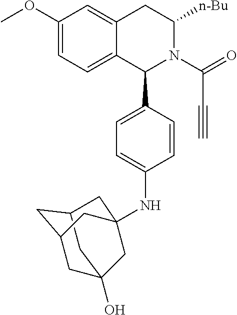 |
| B-14 | 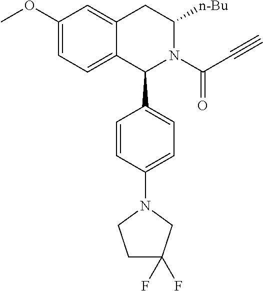 |
| B-15 | 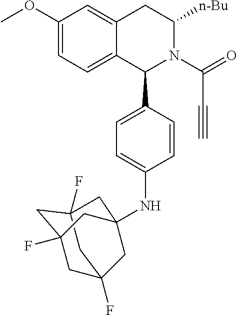 |
| B-16 | 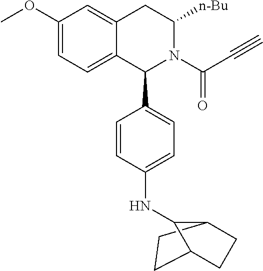 |
| B-17 | 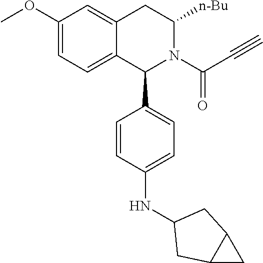 |
| B-18 | 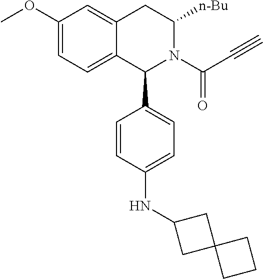 |
| B-19 | 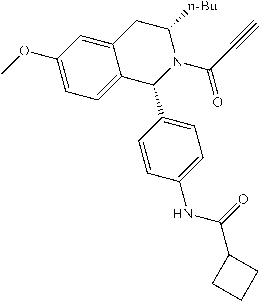 |
| B-20 | 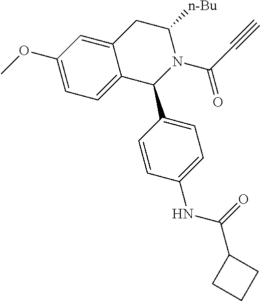 |
| B-21 | 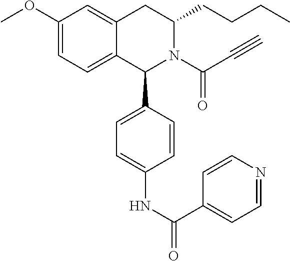 [SMILES: C#CC(=O)N1[C@@H](CCCC)Cc2cc(OC)ccc2[C@@H]1c1ccc(NC(=O)c2ccncc2)cc1] |
| B-22 | 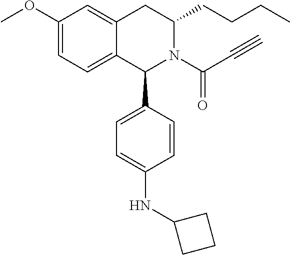 [SMILES: C#CC(=O)N1[C@@H](CCCC)Cc2cc(OC)ccc2[C@@H]1c1ccc(NC2CCC2)cc1] |
| B-24 | 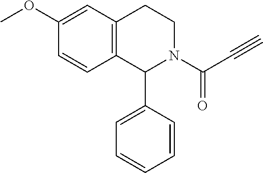 [SMILES: C#CC(=O)N1CCc2cc(OC)ccc2C1c1ccccc1] |
| B-26 | 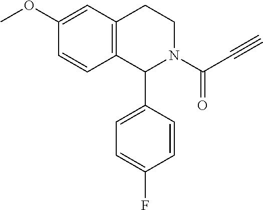 [SMILES: C#CC(=O)N1CCc2cc(OC)ccc2C1c1ccc(F)cc1] |
| B-28 | 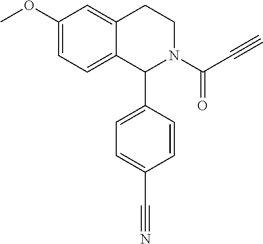 [SMILES: C#CC(=O)N1CCc2cc(OC)ccc2C1c1ccc(C#N)cc1] |
| B-29 | 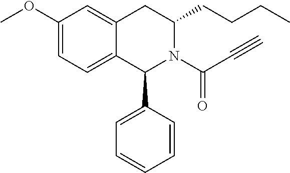 [SMILES: C#CC(=O)N1[C@@H](CCCC)Cc2cc(OC)ccc2[C@@H]1c1ccccc1] |
| B-31 | 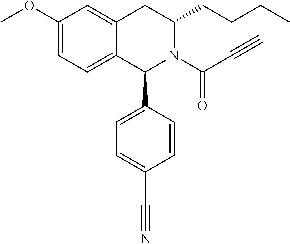 [SMILES: C#CC(=O)N1[C@@H](CCCC)Cc2cc(OC)ccc2[C@@H]1c1ccc(C#N)cc1] |
| B-32 | 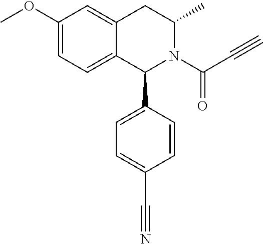 [SMILES: C#CC(=O)N1[C@@H](c2ccc(C#N)cc2)c2ccc(OC)cc2C[C@@H]1C] |
| B-35 | 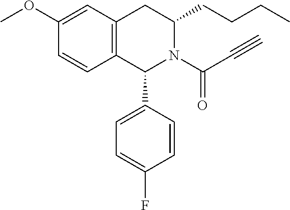 [SMILES: C#CC(=O)N1[C@@H](CCCC)Cc2cc(OC)ccc2[C@H]1c1ccc(F)cc1] |
| B-37 | 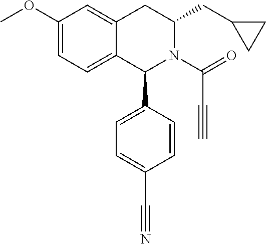 [SMILES: C#CC(=O)N1[C@@H](CC2CC2)Cc2cc(OC)ccc2[C@@H]1c1ccc(C#N)cc1] |
| B-39 | 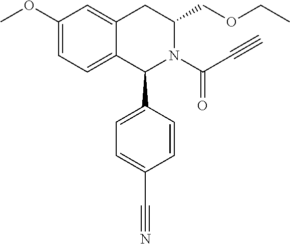 [SMILES: C#CC(=O)N1[C@@H](COCC)Cc2cc(OC)ccc2[C@@H]1c1ccc(C#N)cc1] |
| B-40 | 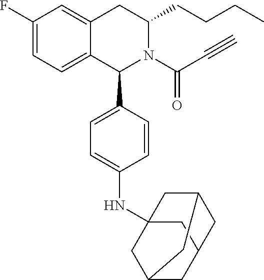 [SMILES: C#CC(=O)N1[C@@H](CCCC)Cc2cc(F)ccc2[C@@H]1c1ccc(NC23CC4CC(CC(C4)C2)C3)cc1] |
| B-41 | 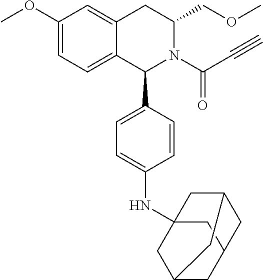 |
| B-42 | 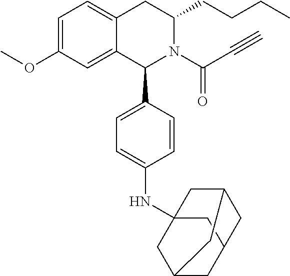 [SMILES: C#CC(=O)N1[C@@H](CCCC)Cc2ccc(OC)cc2[C@@H]1c1ccc(NC23CC4CC(CC(C4)C2)C3)cc1] |
| B-43 | 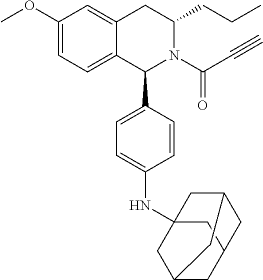 [SMILES: C#CC(=O)N1[C@@H](CCC)Cc2cc(OC)ccc2[C@@H]1c1ccc(NC23CC4CC(CC(C4)C2)C3)cc1] |
| B-44 | 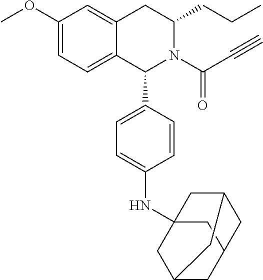 [SMILES: C#CC(=O)N1[C@@H](CCC)Cc2cc(OC)ccc2[C@H]1c1ccc(NC23CC4CC(CC(C4)C2)C3)cc1] |
| B-45 | 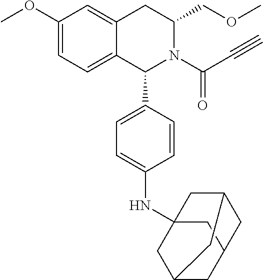 |
| B-47 | 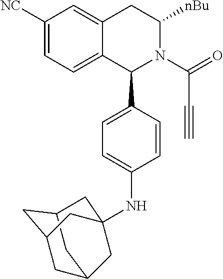 |
| B-48 | 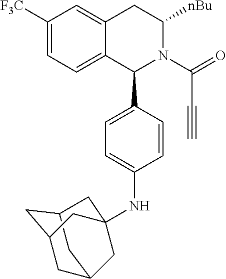 |
| B-49 | 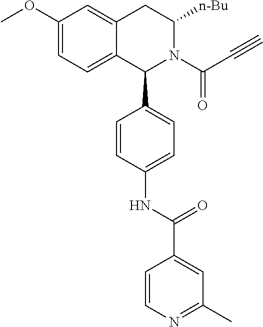 |
| B-50 | 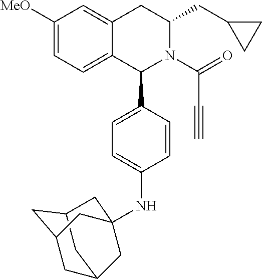 [SMILES: C#CC(=O)N1[C@@H](CC2CC2)Cc2cc(OC)ccc2[C@@H]1c1ccc(NC23CC4CC(CC(C4)C2)C3)cc1] |
| B-51 | 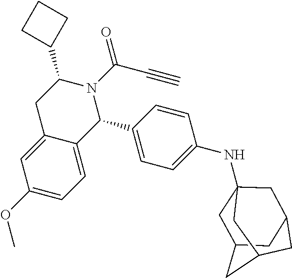 [SMILES: C#CC(=O)N1[C@H](c2ccc(NC34CC5CC(CC(C5)C3)C4)cc2)c2ccc(OC)cc2C[C@@H]1C1CCC1] |
| B-52 | 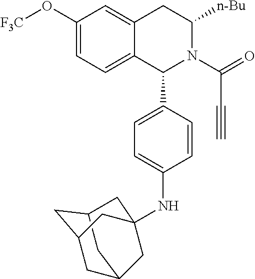 |

In certain embodiments, provided herein is a compound, or a tautomer, stereoisomer, mixture of stereoisomers, isotopically enriched analog, or pharmaceutically acceptable salt thereof, as in Table B-8:

TABLE B-8
Selected compounds of the disclosure.
Structures

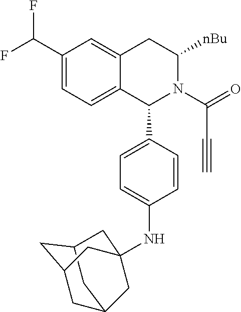

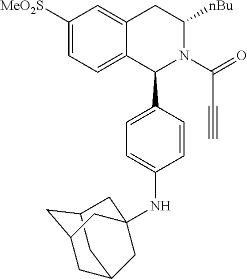

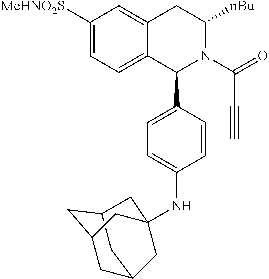

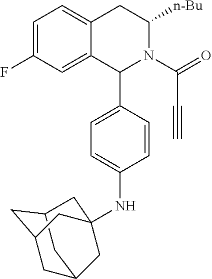

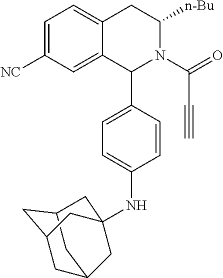

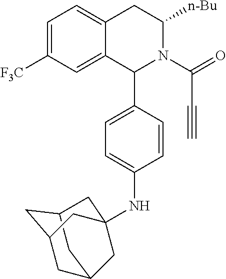

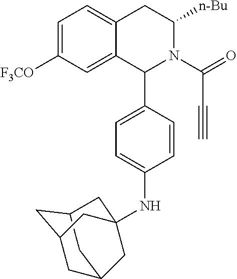

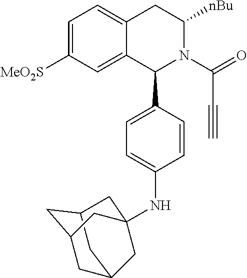

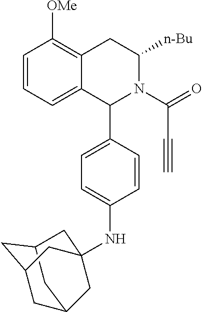

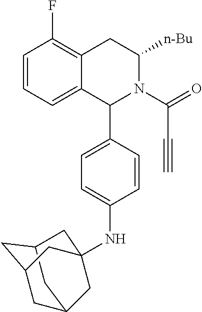

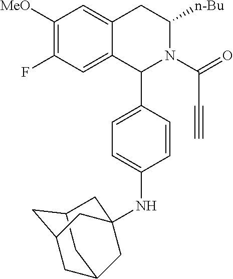

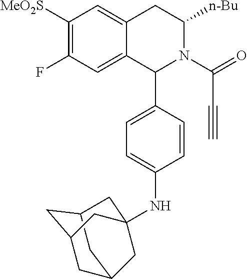

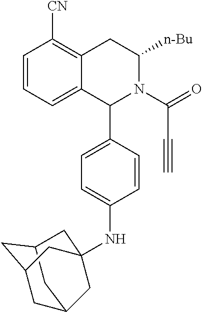

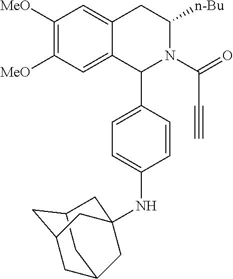

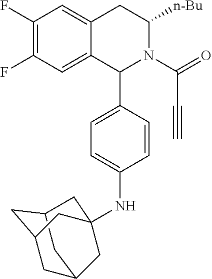

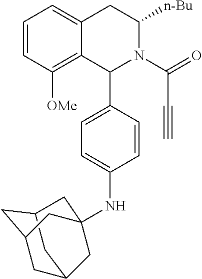

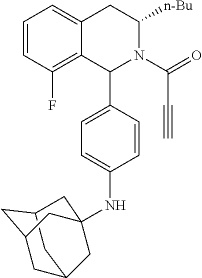

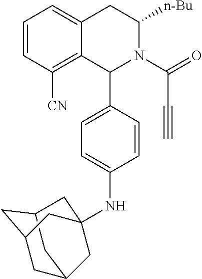

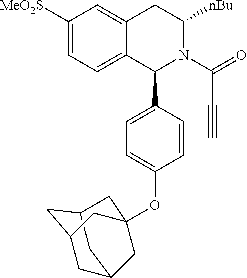

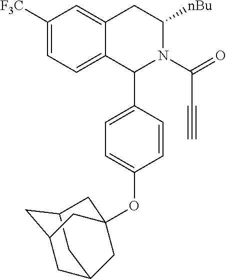

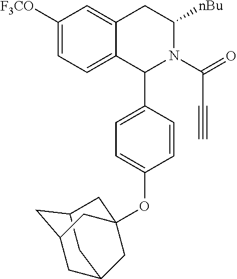

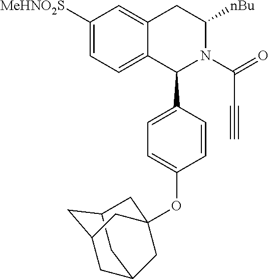

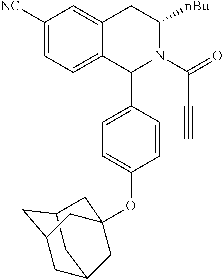

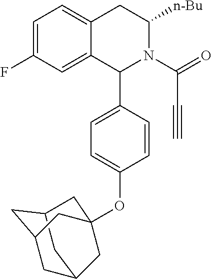

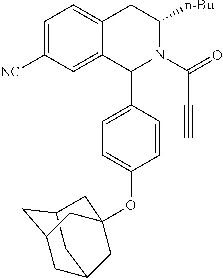

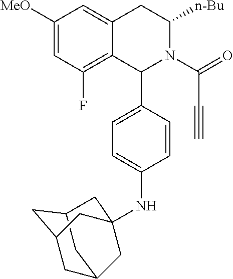

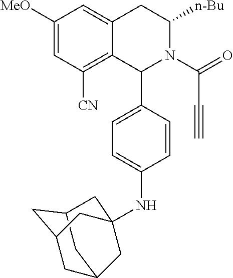

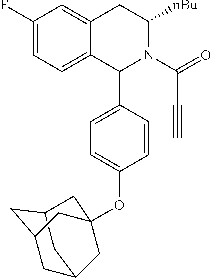

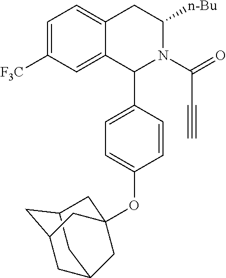

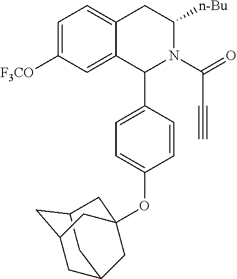

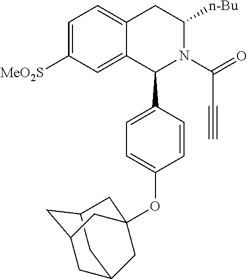

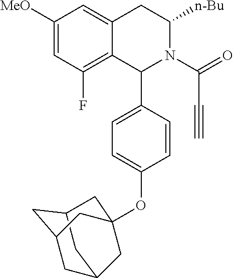

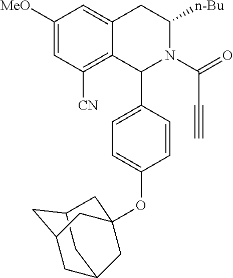

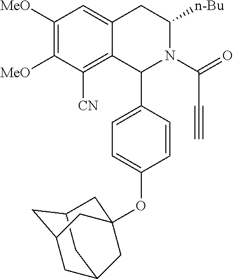

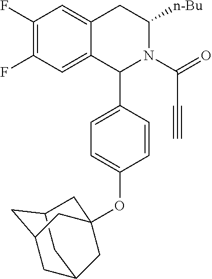

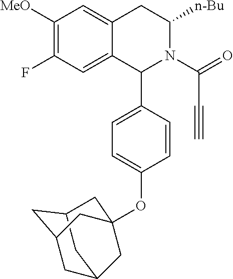

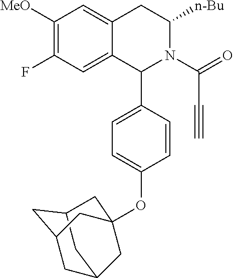

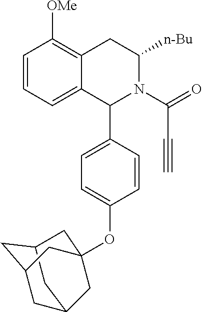

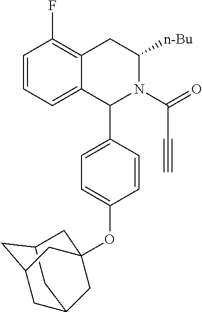

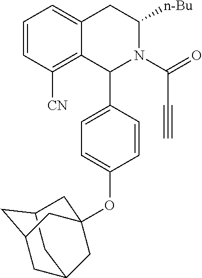

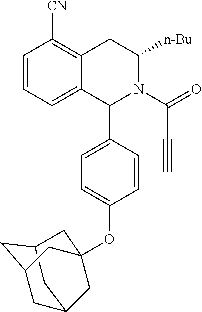

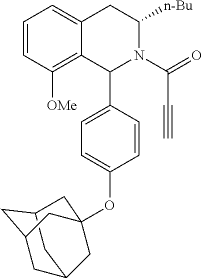

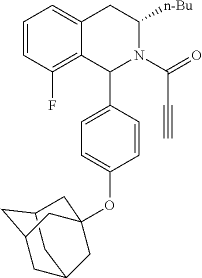

In certain embodiments, provided herein is a compound, or a tautomer, stereoisomer, mixture of stereoisomers, isotopically enriched analog, or pharmaceutically acceptable salt thereof, as in Table B-9:

TABLE B-9
Selected compounds of the disclosure.
Structures

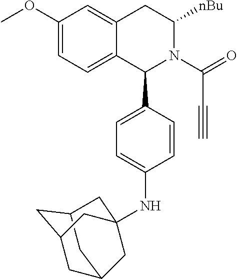

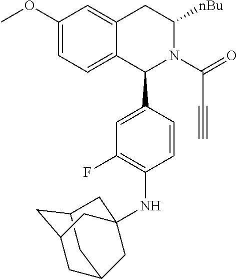

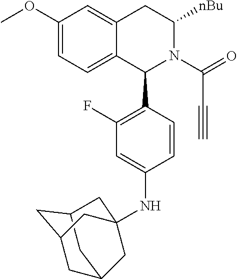

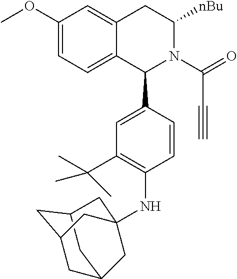

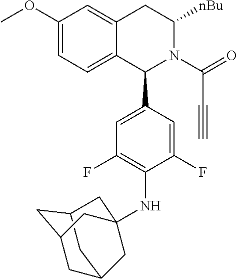

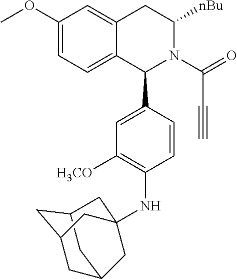

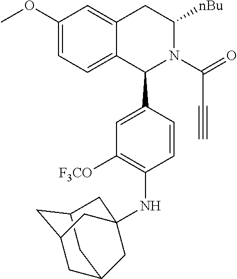

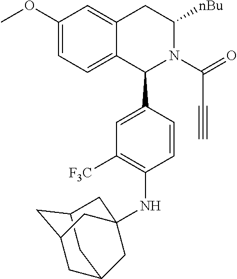

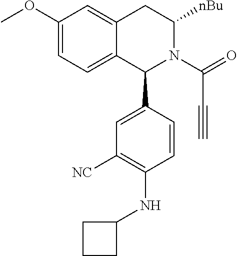

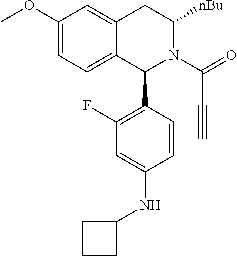

In certain embodiments, provided herein is a compound, or a tautomer, stereoisomer, mixture of stereoisomers, isotopically enriched analog, or pharmaceutically acceptable salt thereof, as in Table B-10:

TABLE B-10
Selected compounds of the disclosure.

| Compound No. | Structure |
|---|---|
| B-53 | 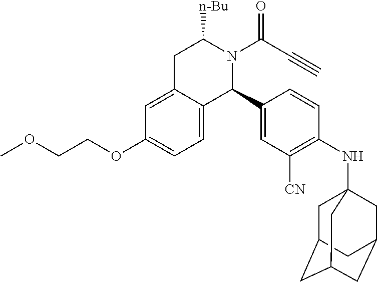 |
| B-54 | 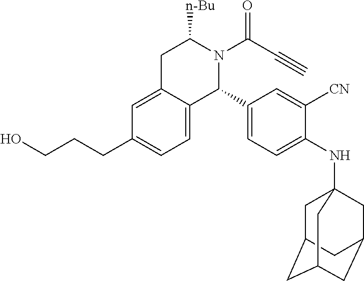 |
| B-55 | 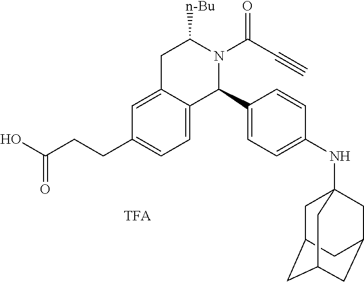 |
| B-56 | 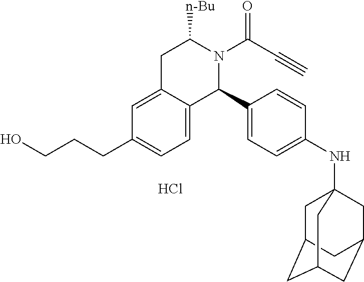 |
| B-57 | 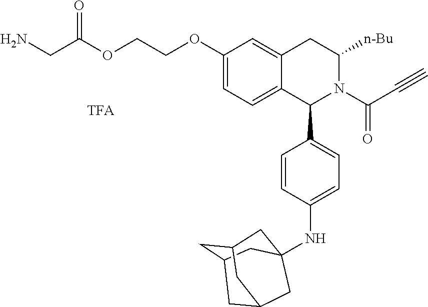 |
| B-58 | 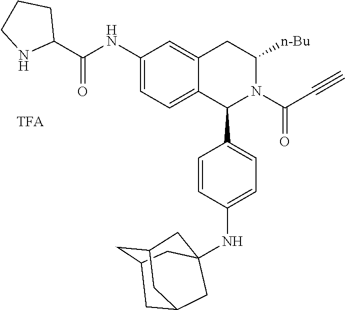 |
| B-59 | 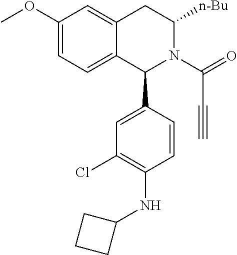 |
| B-60 | 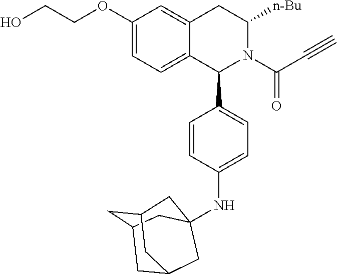 |
| B-61 | 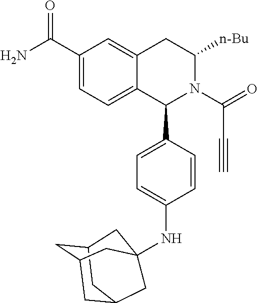 |
| B-62 | 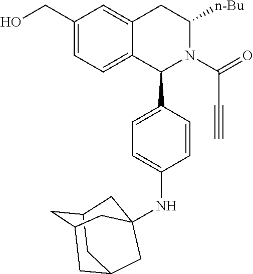 |
| B-63 | 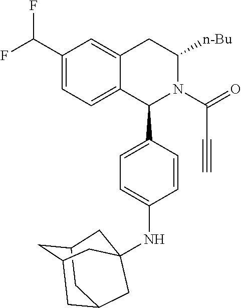 |
| B-64 | 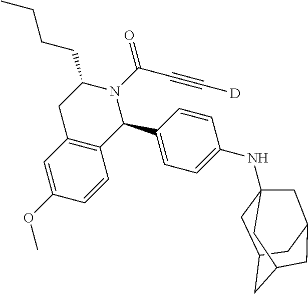 [SMILES: [2H]C#CC(=O)N1[C@@H](CCCC)Cc2cc(OC)ccc2[C@@H]1c1ccc(NC23CC4CC(CC(C4)C2)C3)cc1] |
| B-66 | 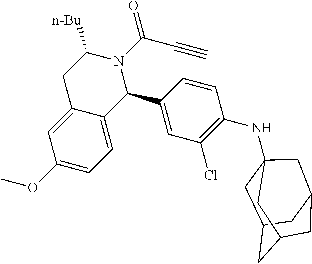 |
| B-67 | 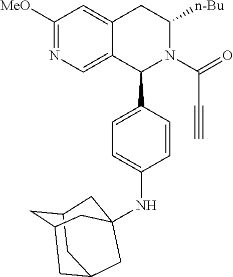 |
| B-68 | 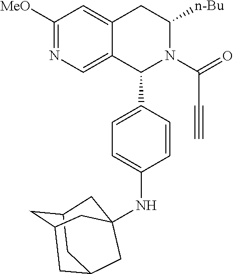 |
| B-69 | 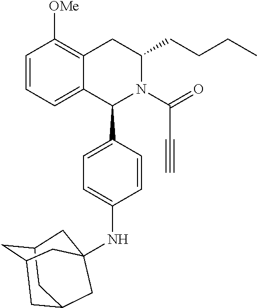 [SMILES: C#CC(=O)N1[C@@H](CCCC)Cc2c(OC)cccc2[C@@H]1c1ccc(NC23CC4CC(CC(C4)C2)C3)cc1] |
| B-65 | 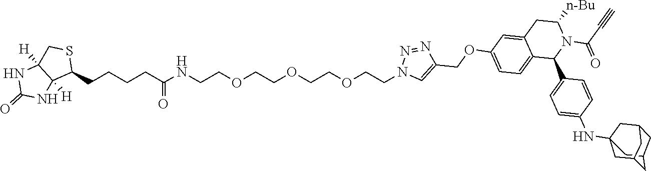 |
| B-70 | 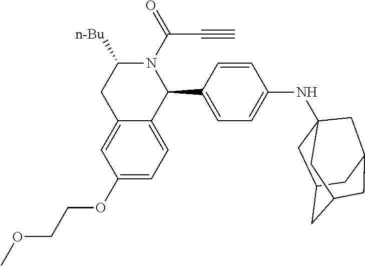 |
| B-71 | 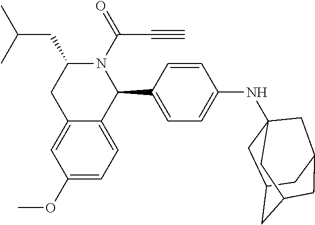 [SMILES: C#CC(=O)N1[C@@H](CC(C)C)Cc2cc(OC)ccc2[C@@H]1c1ccc(NC23CC4CC(CC(C4)C2)C3)cc1] |
| B-72 | 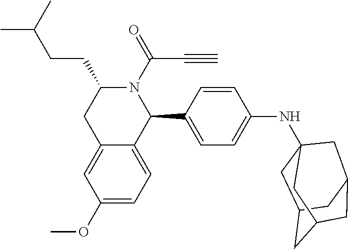 [SMILES: C#CC(=O)N1[C@@H](CCC(C)C)Cc2cc(OC)ccc2[C@@H]1c1ccc(NC23CC4CC(CC(C4)C2)C3)cc1] |
| B-73 | 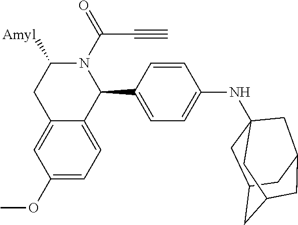 |
| B-74 | 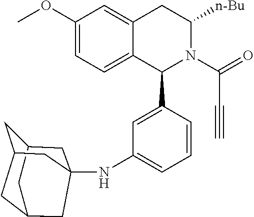 |
| B-75 | 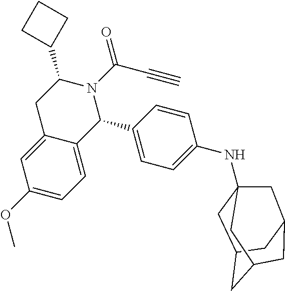 [SMILES: C#CC(=O)N1[C@H](c2ccc(NC34CC5CC(CC(C5)C3)C4)cc2)c2ccc(OC)cc2C[C@@H]1C1CCC1] |
| B-76 | 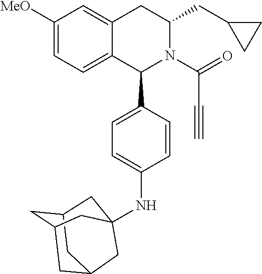 [SMILES: C#CC(=O)N1[C@@H](CC2CC2)Cc2cc(OC)ccc2[C@@H]1c1ccc(NC23CC4CC(CC(C4)C2)C3)cc1] |
| B-77 | 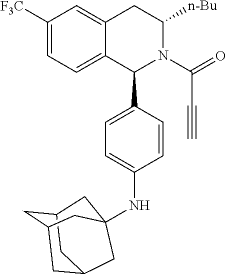 |
| B-78 | 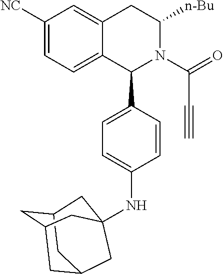 |
| B-79 | 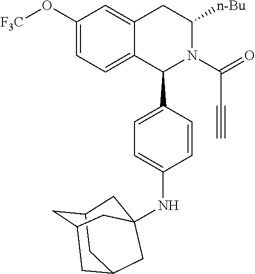 |
| B-80 | 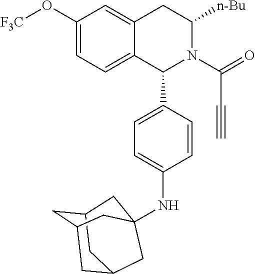 |
| B-81 | 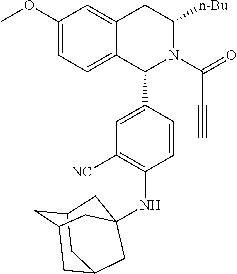 |
| B-82 | 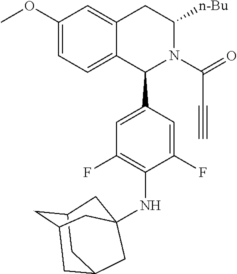 |
| B-83 | 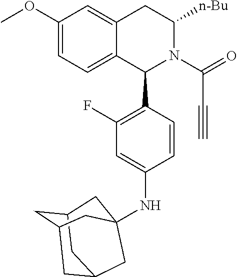 |
| B-84 | 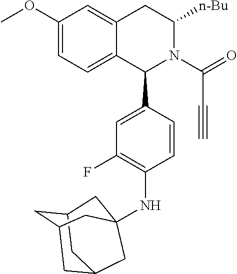 |

In certain embodiments, provided herein is a compound of Formula B-X, or a tautomer, stereoisomer, mixture of stereoisomers, isotopically enriched analog, or pharmaceutically acceptable salt thereof:

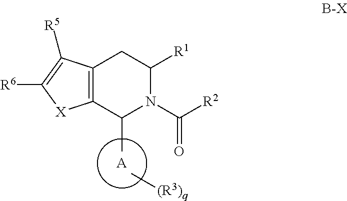

wherein:
ring A is C₄-C₁₀ cycloalkyl, heterocyclyl, aryl, or heteroaryl;
X is —CR¹⁴═CR¹⁴— or —CR¹⁴═N—;
q is 0, 1, 2, or 3;
R¹ is C₁-C₆ alkyl, C₂-C₆ alkenyl, C₂-C₆ alkynyl, C₁-C₆ haloalkyl, C₃-C₁₀ cycloalkyl, —CN, —OR²⁷, —C(O)OR²⁶, —C(O)N(R²⁷)₂, —OC(O)R²⁶, —S(O)₂R²⁸, —S(O)N(R²⁷)₂, —S(O)R²⁸, —N(R²⁷)₂, —NO₂, —C₁-C₆ alkyl-OR²⁷, or —Si(R¹⁵)₃;
R² is

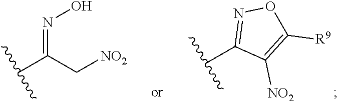

each R³ is independently halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹²)₃, —SF₅, —C(O)OR²⁶, —C(O)N(R²⁷)₂, —NR¹²C(O)R²⁸, —NR¹²C(O)OR²⁸, —OC(O)N(R²⁷)₂, —OC(O)R²⁸, —C(O)R²⁶, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁵ is hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁵ is independently unsubstituted or substituted with one to three R¹⁰;
R⁶ is hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁶ is independently unsubstituted or substituted with one to three R¹⁰;
R⁹ is C₁-C₄ alkyl, C₁-C₄ haloalkyl, or C₂-C₄ alkenyl, wherein each C₁-C₄ alkyl, C₁-C₄ haloalkyl, or C₂-C₄ alkenyl of R⁹ is independently unsubstituted or substituted with one to three R¹¹;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)R¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, C₁-C₆ alkylheterocyclyl, or C₃-C₁₀ cycloalkyl;
each R¹³ is independently C₁-C₆ alkyl or C₃-C₁₀ cycloalkyl;
each R¹⁴ is independently hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkylaryl, —C₂-C₆ alkenylaryl, —C₁-C₆ alkylheteroaryl, or —C₂-C₆ alkenylheteroaryl;
each R²⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R²⁶ is independently unsubstituted or substituted with one to three R¹¹;
each R²⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R²⁷, together with the nitrogen atom to which they are attached, form a 4 to 7 membered heterocyclyl, wherein each R⁷ or ring formed thereby is independently unsubstituted or substituted with one to three R¹¹;
each R²⁸ is independently —(CH₂)ᵤP(O)R^{a}R^{b}, —CH₂)ᵤCH₂OP(O)(R^{a})(R^{b}), C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R²⁸ is independently unsubstituted or substituted with one to three R¹¹;
u is 0, 1, 2, 3, or 4;
each R^{a} is independently selected from the group consisting of hydrogen, —OR¹², —N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently unsubstituted or substituted with one to three R¹¹; and each R⁷ is independently selected from the group consisting of hydrogen, —OR¹², —N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently unsubstituted or substituted with one to three R¹¹; or
R^{a} and R^{b} may combine together to form a ring consisting of 3-8 ring atoms that are C, N, O, or S, wherein the ring is unsubstituted or is substituted with one to three R¹¹.

In certain embodiments, provided herein is a compound of Formula B-X, or a tautomer, stereoisomer, mixture of stereoisomers, isotopically enriched analog, or pharmaceutically acceptable salt thereof, wherein:
ring A is C₄-C₁₀ cycloalkyl, heterocyclyl, aryl, or heteroaryl;
X is —CR¹⁴═CR¹⁴— or —CR¹⁴═N—;
q is 0, 1, 2, or 3;
R¹ is C₁-C₆ alkyl, C₂-C₆ alkenyl, C₂-C₆ alkynyl, C₁-C₆ haloalkyl, C₃-C₁₀ cycloalkyl, —CN, —OR²⁷, —C(O)OR²⁶, —C(O)N(R²⁷)₂—OC(O)R²⁶, —S(O)₂R²⁸, —S(O)₂N(R²⁷)₂, —S(O)N(R²⁷)₂, —S(O)R²⁸, —N(R²⁷)₂, —NO₂, —C₁-C₆ alkyl-OR²⁷, or —Si(R¹⁵)₃;
R² is

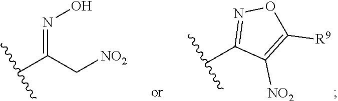

each R³ is independently halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹²)₃, —SF₅, —C(O)OR²⁶, —C(O)N(R²⁷)₂, —NR¹²C(O)R²⁸, —NR¹²C(O)OR²⁸, —OC(O)N(R²⁷)₂, —OC(O)R²⁸, —C(O)R²⁶, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁵ is hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁵ is independently unsubstituted or substituted with one to three R¹⁰;
R⁶ is hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁶ is independently unsubstituted or substituted with one to three R¹⁰;
R⁹ is C₁-C₄ alkyl, C₁-C₄ haloalkyl, or C₂-C₄ alkenyl, wherein each C₁-C₄ alkyl, C₁-C₄ haloalkyl, or C₂-C₄ alkenyl of R⁹ is independently unsubstituted or substituted with one to three R¹¹;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkylaryl, —C₂-C₆ alkenylaryl, —C₁-C₆ alkylheteroaryl, or —C₂-C₆ alkenylheteroaryl;
each R²⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R²⁶ is independently unsubstituted or substituted with one to three R¹¹;
each R²⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R²⁷, together with the nitrogen atom to which they are attached, form a 4 to 7 membered heterocyclyl, wherein each R²⁷ or ring formed thereby is independently unsubstituted or substituted with one to three R¹¹;
each R²⁸ is independently —(CH₂)ᵤP(O)R^{a}R^{b}, —CH₂)ᵤCH₂OP(O)(R^{a})(R), C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R²⁸ is independently unsubstituted or substituted with one to three R¹¹;
u is 0, 1, 2, 3, or 4;
each R^{a} is independently selected from the group consisting of hydrogen, —OR¹², —N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently unsubstituted or substituted with one to three R¹¹; and
each R^{b} is independently selected from the group consisting of hydrogen, —OR¹², —N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently unsubstituted or substituted with one to three R¹¹; or
R^{a} and R^{b} may combine together to form a ring consisting of 3-8 ring atoms that are C, N, O, or S, wherein the ring is unsubstituted or is substituted with one to three R¹¹.

In certain embodiments, each R²⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R²⁶ is independently further substituted with one to three R¹¹;
each R²⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R²⁷, together with the nitrogen atom to which they are attached, form a 4 to 7 membered heterocyclyl, wherein each R²⁷ or ring formed thereby is independently further substituted with one to three R¹¹;
each R²⁸ is independently —(CH₂)ᵤP(O)R^{a}R^{b}, —CH₂)ᵤCH₂OP(O)(R^{a})(R), C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R²⁸ is independently further substituted with one to three R¹¹;
u is 0, 1, 2, 3, or 4;
each R^{a} is independently selected from the group consisting of hydrogen, —OR¹², —N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently further substituted with one to three R¹¹; and
each R^{b} is independently selected from the group consisting of hydrogen, —OR¹², —N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently further substituted with one to three R¹¹; or
R^{a} and R^{b} may combine together to form a ring consisting of 3-8 ring atoms that are C, N, O, or S wherein the ring is unsubstituted or is substituted with one to three R¹¹.

In certain embodiments, R¹ is C₁-C₆ alkyl, C₂-C₆ alkenyl, C₂-C₆ alkynyl, C₁-C₆ haloalkyl, C₃-C₁₀ cycloalkyl, —CN, —C(O)OR²⁶, —C(O)N(R²⁷)₂, —N(R²⁷)₂, —OR²⁷, or —C₁-C₆ alkyl-OR²⁷.

In certain embodiments, R¹ is C₁-C₆ alkyl, C₃-C₁₀ cycloalkyl, or —C₁-C₆ alkyl-OR²⁷.

In certain embodiments, R¹ is C₁-C₄ alkyl.

In certain embodiments, R⁹ is C₁-C₄ alkyl.

In certain embodiments R² is:

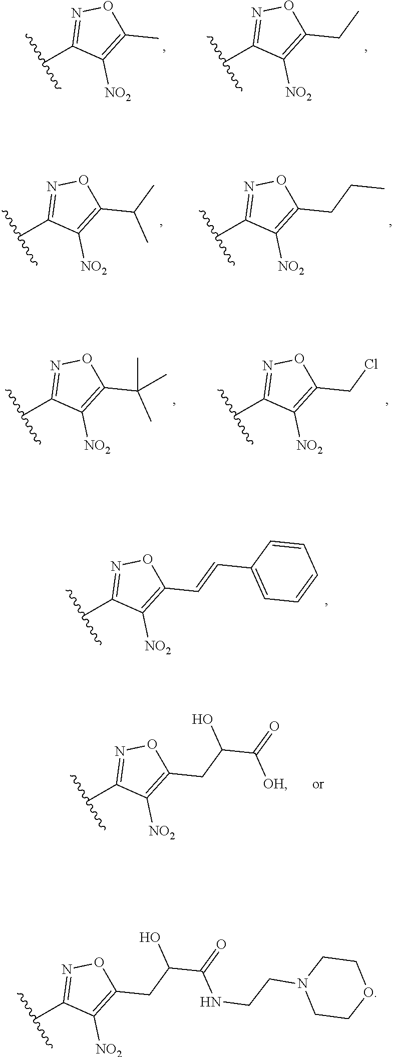

In certain embodiments, R³ is C₃-C₁₀ cycloalkyl.

In certain embodiments, R⁶ is halo, —CN, —OR²⁸, —S(O)₂R²⁸, —S(O)₂N(R²⁷)₂, or C₁-C₆ alkyl, wherein each C₁-C₆ alkyl of R⁶ is independently unsubstituted or substituted with one to three R¹⁰.

In certain embodiments, R⁶ is —CN, —F, CF₂H, —OCH₃, —OCF₃, —CF₃, —S(O)₂CH₃, or —S(O)₂NHCH₃.

In certain embodiments, R⁵ is hydrogen.

In certain embodiments, one R¹⁴ is hydrogen and the other R¹⁴ is hydrogen, halo, —CN, —OR²⁸, —S(O)₂R²⁸, —S(O)₂N(R²⁷)₂, or C₁-C₆ alkyl, wherein each C₁-C₆ alkyl of R¹⁴ is independently unsubstituted or substituted with one to three R¹⁰.

In certain embodiments, ring A is aryl or heteroaryl. In certain embodiments, ring A is a monocyclic aryl or monocyclic heteroaryl. In certain embodiments, ring A is heterocyclyl. In certain embodiments, ring A is a 4 to 7 membered heterocyclyl. In certain embodiments, ring A is aryl. In certain embodiments, ring A is phenyl. In certain embodiments, ring A is heteroaryl. In certain embodiments, ring A is pyridyl. In certain embodiments, ring A is phenyl, pyridyl, piperidynyl, piperazinyl, or morpholinyl.

In certain embodiments, ring A is aryl or heteroaryl, any of which is substituted by one to three R³. In certain embodiments, ring A is aryl or heteroaryl, any of which is substituted by one to three R³, where at least one R³ is C₃-C₁₀ cycloalkyl, heterocyclyl, aryl, or heteroaryl, wherein each C₃-C₁₀ cycloalkyl, heterocyclyl, aryl, and heteroaryl of R³ is unsubstituted or is substituted with one to three R¹⁰.

In certain embodiments, ring A is aryl or heteroaryl, any of which is substituted by two or three R³. In certain embodiments, ring A is aryl or heteroaryl, any of which is substituted by two or three R³, wherein at least one R³ is halo.

In certain embodiments, ring A is:

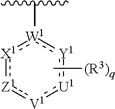

wherein 0 to 3 of U¹, V¹, W¹, X¹, Y¹, and Z¹ is independently N, S, or O, and the remaining variables are CH or CR³ and each [symbol] independently represents a single or double bond, which comply with valency requirements based on U¹, V¹, W¹, X¹, Y¹, and Z¹.

In certain embodiments, ring A is:

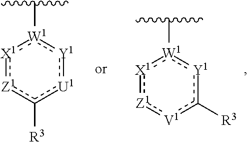

wherein 1 to 3 of U¹, W¹, X¹, Y¹, and Z¹ is N, S, or O, and the remaining variables are CH or CR³ and [symbol] represents a single or double bond, which comply with valency requirements based on U¹, W¹, X¹, Y¹, and Z¹.

In certain embodiments, ring A is aryl or heteroaryl. In certain embodiments, ring A is a monocyclic aryl or monocyclic heteroaryl. In certain embodiments, ring A is heterocyclyl. In certain embodiments, ring A is a 4 to 7 membered heterocyclyl. In certain embodiments, ring A is aryl. In certain embodiments, ring A is phenyl. In certain embodiments, ring A is heteroaryl. In certain embodiments, ring A is pyridyl. In certain embodiments, ring A is pyrazolyl. In certain embodiments, ring A is phenyl, pyridyl, piperidynyl, piperazinyl, or morpholinyl.

In certain embodiments, ring A is aryl or heteroaryl, any of which is substituted by one to three R. In certain embodiments, ring A is aryl or heteroaryl, any of which is substituted by one to three R³, where at least one R³ is C₃-C₁₀ cycloalkyl, heterocyclyl, aryl, or heteroaryl, wherein each C₃-C₁₀ cycloalkyl, heterocyclyl, aryl, and heteroaryl of R³ is unsubstituted or is substituted with one to three R¹⁰.

In certain embodiments, ring A is aryl or heteroaryl, any of which is substituted by two or three R³. In certain embodiments, ring A is aryl or heteroaryl, any of which is substituted by two or three R³, wherein at least one R³ is halo.

In certain embodiments, ring A is cyclohexyl. In certain embodiments, ring A is C₄-C₁₀ cycloalkyl. In certain embodiments, ring A is a C₄-C₇ cycloalkyl. In certain embodiments, ring A is bicyclo[1.1.1]pentanyl. In certain embodiments, ring A is selected from cyclobutyl, cyclopentyl, cyclohexyl, and cycloheptyl.

In certain embodiments, ring A is:

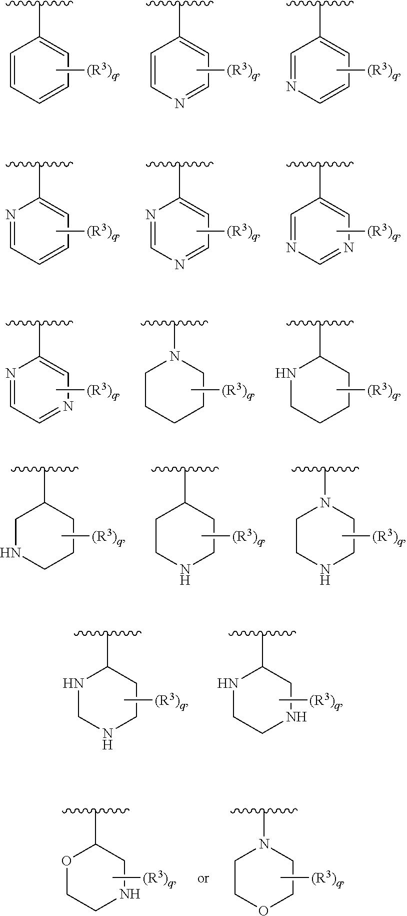

where q and each R³ is independently as defined herein.

In certain embodiments, ring A is:

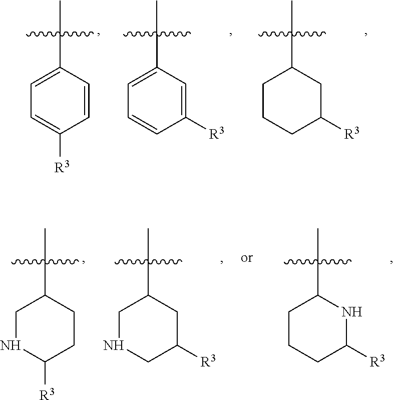

where R³ is independently as defined herein.

In certain embodiments, ring A is a bridged bicyclic ring selected from:

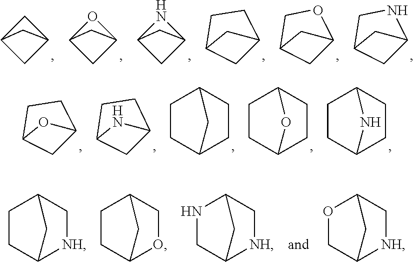

wherein each is unsubstituted or substituted with one to three R³. In certain embodiments, ring A is a bridged bicyclic ring selected from:

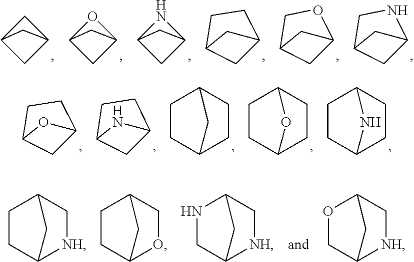

wherein each is substituted with one to three R³. In certain embodiments, ring A is a bridged bicyclic ring selected from:

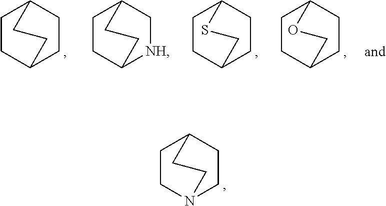

wherein each R³ is attached to a carbon atom on the bridged bicyclic ring.

In certain embodiments, ring A is:

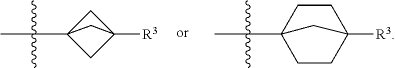

In certain embodiments, at least one R³ is halo, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹²)₃, —SF₅, —C(O)OR²⁶, —C(O)N(R²⁷)₂, —NR¹²C(O)R²⁸, —NR¹²C(O)OR²⁸, —OC(O)R²⁸, —C(O)R²⁶, or —OC(O)CHR²⁸N(R¹²)₂.

In certain embodiments, at least one R³ is —NHR²⁸, —OH, —OR²⁸, —S(O)₂R²⁸, —S(O)R²⁸, —NR¹²C(O)R²⁸, —NR¹²C(O)OR²⁸, —OC(O)R²⁸, or —OC(O)CHR²⁸N(R¹²)₂.

In certain embodiments, at least one R³ is halo.

In certain embodiments, at least one R³ is —NHR²⁸. In certain embodiments, at least one R³ is —N(R²⁸)₂. In certain embodiments, q is 2, and one R²⁸ is halo or —CN, and the other R²⁸ is —N(R²⁸)₂. In certain embodiments, q is 2, and one R³ is halo and the other R³ is —N(R²⁸)₂. In certain embodiments, q is 3, and two R³ are independently halo and one R³ is —N(R²⁸)₂.

In certain embodiments, at least one R³ is —NHR²⁸ or —OR²⁸.

In certain embodiments, at least one R³ is —C(O)OR²⁶ or —C(O)R²⁶.

In certain embodiments, at least one R³ is —S(O)₂N(R²⁷)₂, —S(O)N(R²⁷)₂, or —C(O)N(R²⁷)₂.

In certain embodiments, at least one R³ is —S(O)₂R²⁸, —S(O)R²⁸, —NR¹²C(O)R²⁸, —NR¹²C(O)OR²⁸, —OC(O)R²⁸, or —OC(O)CHR²⁸N(R¹²)₂.

In certain embodiments, each R³ is independently halo, —CN, —OH, —OR²⁸, —NHR²⁸, —S(O)₂R²⁸, —S(O)₂N(R²⁷)₂, —NO₂, —Si(R¹²)₃, —SF₅, —C(O)OR²⁶, —C(O)N(R²⁷)₂, —NR¹²C(O)R²⁸, —NR¹²C(O)OR²⁸, —OC(O)R²⁸, —OC(O)CHR²⁸N(R¹²)₂, C₁-C₆ alkyl, C₃-C₁₀ cycloalkyl, heterocyclyl, heteroaryl, or —C₁-C₆ alkylheterocyclyl, wherein each C₁-C₆ alkyl, C₃-C₁₀ cycloalkyl, heterocyclyl, heteroaryl, or —C₁-C₆ alkylheterocyclyl of R³ is independently unsubstituted or substituted with one to three R¹⁰.

In certain embodiments, each R³ is independently halo, —CN, —OH, —OR²⁸, —NHR²⁸, —S(O)₂R²⁸, —S(O)₂N(R²⁷)₂, —NO₂, —Si(R¹²)₃, —SF₅, —C(O)OR²⁶, —C(O)N(R²⁷)₂, —NR¹²C(O)R²⁸, —NR¹²C(O)OR²⁸, —OC(O)R²⁸, —OC(O)CHR²⁸N(R¹²)₂, C₁-C₆ alkyl, C₃-C₁₀ cycloalkyl, heterocyclyl, heteroaryl, or —C₁-C₆ alkylheterocyclyl, wherein each C₁-C₆ alkyl, C₃-C₁₀ cycloalkyl, heterocyclyl, heteroaryl, or —C₁-C₆ alkylheterocyclyl is independently unsubstituted or substituted with one to three substituents independently selected from —OR¹², —N(R¹²)₂, —S(O)₂R¹³, —OC(O)CHR¹²N(R¹²)₂, and C₁-C₆ alkyl that is unsubstituted or is substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, R⁵ is hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, C₁-C₆ alkyl, C₂-C₆ alkenyl, C₂-C₆ alkynyl, or C₃-C₁₀ cycloalkyl, wherein each C₁-C₆ alkyl, C₂-C₆ alkenyl, C₂-C₆ alkynyl, or C₃-C₁₀ cycloalkyl of R⁵ is independently unsubstituted or substituted with one to three R¹⁰.

In certain embodiments, R⁵ is hydrogen, halo, —CN, —OH, —OR²⁸, C₁-C₆ alkyl, C₂-C₆ alkynyl, or C₃-C₁₀ cycloalkyl, wherein each C₁-C₆ alkyl, C₂-C₆ alkynyl, or C₃-C₁₀ cycloalkyl of R⁵ is independently unsubstituted or substituted with one to three R¹⁰.

In certain embodiments, R⁵ is hydrogen, halo, —CN, —OH, —OR²⁸, C₁-C₆ alkyl, C₂-C₆ alkynyl, or C₃-C₁₀ cycloalkyl.

In certain embodiments, R⁵ is hydrogen, halo, —CN, —OH, —OR²⁸, C₁-C₆ alkyl, or C₂-C₆ alkynyl, wherein the C₁-C₆ alkyl of R⁵ is unsubstituted or is substituted with one to three R¹⁰.

In certain embodiments, R⁵ is hydrogen, halo, —CN, —OH, —OR²⁸, C₁-C₆ alkyl, C₂-C₆ alkynyl, wherein the C₁-C₆ alkyl of R⁵ is unsubstituted or is substituted with one to three substituents independently selected from —OR¹², —N(R¹²)₂, —S(O)₂R¹³, —OC(O)CHR¹²N(R¹²)₂, and C₁-C₆ alkyl that is unsubstituted or is substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
R²⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R²⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, R⁶ is halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, C₁-C₆ alkyl, C₂-C₆ alkenyl, C₂-C₆ alkynyl, or C₃-C₁₀ cycloalkyl, wherein each C₁-C₆ alkyl, C₂-C₆ alkenyl, C₂-C₆ alkynyl, or C₃-C₁₀ cycloalkyl of R¹⁸ is independently unsubstituted or substituted with one to three R¹⁰.

In certain embodiments, R⁶ is halo, —CN, —OH, —OR²⁸, C₁-C₆ alkyl, C₂-C₆ alkynyl, or C₃-C₁₀ cycloalkyl, wherein each C₁-C₆ alkyl, C₂-C₆ alkynyl, or C₃-C₁₀ cycloalkyl of R⁶ is independently unsubstituted or substituted with one to three R¹⁰.

In certain embodiments, R⁶ is halo, —CN, —OH, —OR²⁸, C₁-C₆ alkyl, C₂-C₆ alkynyl, or C₃-C₁₀ cycloalkyl.

In certain embodiments, R⁶ is halo, —CN, —OH, —OR²⁸, C₁-C₆ alkyl, or C₂-C₆ alkynyl, wherein the C₁-C₆ alkyl of R⁶ is unsubstituted or is substituted with one to three R¹⁰.

In certain embodiments, R⁶ is halo, —CN, —OH, —OR²⁸, C₁-C₆ alkyl, C₂-C₆ alkynyl, wherein the C₁-C₆ alkyl of R⁶ is unsubstituted or is substituted with one to three substituents independently selected from —OR¹², —N(R¹²)₂, —S(O)₂R¹³, —OC(O)CHR¹²N(R¹²)₂, and C₁-C₆ alkyl that is unsubstituted or is substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein

R²⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R²⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, R⁵ is hydrogen, halo, —CN, —OH, —OR²⁸, C₁-C₆ alkyl, C₂-C₆ alkynyl, or C₃-C₁₀ cycloalkyl, wherein each C₁-C₆ alkyl, C₂-C₆ alkynyl, or C₃-C₁₀ cycloalkyl of R⁵ is independently unsubstituted or substituted with one to three R¹⁰.

In certain embodiments, each R¹⁴ is independently hydrogen, halo, —CN, —OH, —OR²⁸, C₁-C₆ alkyl, C₂-C₆ alkynyl, or C₃-C₁₀ cycloalkyl.

In certain embodiments, each R¹⁴ is independently hydrogen, halo, —CN, —OH, —OR²⁸, C₁-C₆ alkyl, or C₂-C₆ alkynyl, wherein the C₁-C₆ alkyl of R¹⁴ is unsubstituted or is substituted with one to three R¹⁰.

In certain embodiments, each R²⁶ is independently hydrogen, C₁-C₆ alkyl, C₂-C₆ alkenyl, or —C₁-C₆ alkylC₃-C₁₀ cycloalkyl, wherein each R²⁶ is independently further substituted with one to three R¹¹.

In certain embodiments, each R²⁶ is independently hydrogen, C₁-C₆ alkyl, C₂-C₆ alkenyl, or —C₁-C₆ alkylC₃-C₁₀ cycloalkyl, wherein each R²⁶ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
each R¹² is independently hydrogen, C₁-C₆ alkyl, or C₃-C₁₀ cycloalkyl.

In certain embodiments, each R²⁷ is independently hydrogen, C₁-C₆ alkyl, C₃-C₁₀ cycloalkyl, heterocyclyl, heteroaryl, —C₁-C₆ alkylC₃-C₆ cycloalkyl, —C₁-C₆ alkylheterocyclyl, or two R²⁷, together with the nitrogen atom to which they are attached, form a 4 to 7 membered heterocyclyl, wherein each R²⁷ or ring formed thereby is independently further substituted with one to three R¹¹.

In certain embodiments, each R²⁷ is independently hydrogen, C₁-C₆ alkyl, C₃-C₁₀ cycloalkyl, heterocyclyl, heteroaryl, —C₁-C₆ alkylC₃-C₆ cycloalkyl, —C₁-C₆ alkylheterocyclyl, or two R²⁷, together with the nitrogen atom to which they are attached, form a 4 to 7 membered heterocyclyl, wherein each R²⁷ or ring formed thereby is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
each R¹² is independently hydrogen, C₁-C₆ alkyl, or C₃-C₁₀ cycloalkyl.

In certain embodiments, each R²⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R²⁸ is independently further substituted with one to three R¹¹.

In certain embodiments, each R²⁸ is independently C₁-C₆ alkyl, C₂-C₆ alkynyl, C₃-C₁₀ cycloalkyl, —C₁-C₆ alkylC₃-C₁₀ cycloalkyl, or —C₁-C₆ alkylaryl, wherein each R²⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆ alkyl, or heterocyclyl; wherein
each R¹² is independently hydrogen, C₁-C₆ alkyl, or C₃-C₁₀ cycloalkyl.

In certain embodiments, R²⁸ is C₁-C₆ alkyl. In certain embodiments, R²⁸ is C₃-C₁₀ cycloalkyl. In certain embodiments, R²⁸ is —C₁-C₆ alkylC₃-C₁₀ cycloalkyl. In certain embodiments, R²⁸ is C₁-C₆ alkyl, C₂-C₆ alkynyl, C₃-C₁₀ cycloalkyl, or —C₁-C₆ alkylC₃-C₁₀ cycloalkyl.

In certain embodiments, each R¹⁰ is independently —OR¹², —N(R¹²)₂, —S(O)₂R¹³, —OC(O)CHR¹²N(R¹²)₂, or C₁-C₆ alkyl, wherein the C₁-C₆ alkyl, of R¹⁵ is independently unsubstituted or substituted with one to three R¹¹;
each R¹¹ is independently halo, —OR¹², —N(R¹²)₂, —Si(R¹²)₃, —C(O)OR¹², —NR¹²C(O)R¹², —OC(O)CHR¹²N(R¹²)₂, C₁-C₆ alkyl, or heterocyclyl;
each R¹² is independently hydrogen, C₁-C₆ alkyl, or C₃-C₁₀ cycloalkyl; and
each R¹³ is independently C₁-C₆ alkyl or C₃-C₁₀ cycloalkyl.

In certain embodiments, each R¹⁵ is independently C₁-C₆ alkyl.

Also provided is a compound, or a tautomer, stereoisomer, mixture of stereoisomers, isotopically enriched analog, or pharmaceutically acceptable salt thereof, represented by Formula B-XIA, as in Table B-XIA:

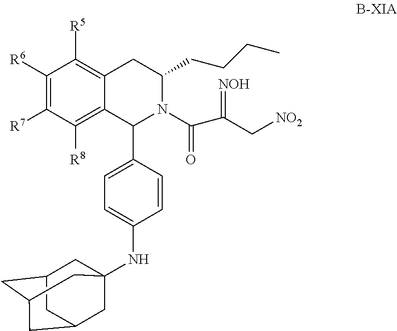

TABLE B-XIA

| No. | R⁵ | R⁶ | R⁷ | R⁸ |
|---|---|---|---|---|
| B-XIA-1 | —OCH₃ | —H | —H | —H |
| B-XIA-2 | —H | —OCH₃ | —H | —H |
| B-XIA-3 | —H | —H | —OCH₃ | —H |
| B-XIA-4 | —H | —H | —H | —OCH₃ |
| B-XIA-5 | —OCF₃ | —H | —H | —H |
| B-XIA-6 | —H | —OCF₃ | —H | —H |
| B-XIA-7 | —H | —H | —OCF₃ | —H |
| B-XIA-8 | —H | —H | —H | —OCF₃ |
| B-XIA-9 | —CH₃ | —H | —H | —H |
| B-XIA-10 | —H | —CH₃ | —H | —H |
| B-XIA-11 | —H | —H | —CH₃ | —H |
| B-XIA-12 | —H | —H | —H | —CH₃ |
| B-XIA-13 | —CF₃ | —H | —H | —H |
| B-XIA-14 | —H | —CF₃ | —H | —H |
| B-XIA-15 | —H | —H | —CF₃ | —H |
| B-XIA-16 | —H | —H | —H | —CF₃ |
| B-XIA-17 | —CN | —H | —H | —H |
| B-XIA-18 | —H | —CN | —H | —H |
| B-XIA-19 | —H | —H | —CN | —H |
| B-XIA-20 | —H | —H | —H | —CN |
| B-XIA-21 | —F | —H | —H | —H |
| B-XIA-22 | —H | —F | —H | —H |
| B-XIA-23 | —H | —H | —F | —H |
| B-XIA-24 | —H | —H | —H | —F |
| B-XIA-25 | —CF₂H | —H | —H | —H |
| B-XIA-26 | —H | —CF₂H | —H | —H |
| B-XIA-27 | —H | —H | —CF₂H | —H |
| B-XIA-28 | —H | —H | —H | —CF₂H |
| B-XIA-29 | —SO₂CH₃ | —H | —H | —H |
| B-XIA-30 | —H | —SO₂CH₃ | —H | —H |
| B-XIA-31 | —H | —H | —SO₂CH₃ | —H |
| B-XIA-32 | —H | —H | —H | —SO₂CH₃ |
| B-XIA-33 | —SO₂NHCH₃ | —H | —H | —H |
| B-XIA-34 | —H | —SO₂NHCH₃ | —H | —H |
| B-XIA-35 | —H | —H | —SO₂NHCH₃ | —H |
| B-XIA-36 | —H | —H | —H | —SO₂NHCH₃ |
| B-XIA-37 | —OCH₃ | —OCH₃ | —H | —H |
| B-XIA-38 | —H | —OCH₃ | —OCH₃ | —H |
| B-XIA-39 | —H | —OCH₃ | —H | —OCH₃ |
| B-XIA-40 | —OCH₃ | —H | —OCH₃ | —H |
| B-XIA-41 | —H | —H | —OCH₃ | —OCH₃ |
| B-XIA-42 | —OCH₃ | —H | —H | —OCH₃ |
| B-XIA-43 | —F | —OCH₃ | —H | —H |
| B-XIA-44 | —H | —OCH₃ | —F | —H |
| B-XIA-45 | —H | —OCH₃ | —H | —F |
| B-XIA-46 | —F | —H | —OCH₃ | —H |
| B-XIA-47 | —H | —F | —OCH₃ | —H |
| B-XIA-48 | —H | —H | —OCH₃ | —F |
| B-XIA-49 | —OCH₃ | —F | —H | —H |
| B-XIA-50 | —OCH₃ | —H | —F | —H |
| B-XIA-51 | —OCH₃ | —H | —H | —F |
| B-XIA-52 | —F | —H | —H | —OCH₃ |
| B-XIA-53 | —H | —F | —H | —OCH₃ |
| B-XIA-54 | —H | —H | —F | —OCH₃ |
| B-XIA-55 | —CN | —OCH₃ | —H | —H |
| B-XIA-56 | —H | —OCH₃ | —CN | —H |
| B-XIA-57 | —H | —OCH₃ | —H | —CN |
| B-XIA-58 | —CN | —H | —OCH₃ | —H |
| B-XIA-59 | —H | —CN | —OCH₃ | —H |
| B-XIA-60 | —H | —H | —OCH₃ | —CN |
| B-XIA-61 | —OCH₃ | —CN | —H | —H |
| B-XIA-62 | —OCH₃ | —H | —CN | —H |
| B-XIA-63 | —OCH₃ | —H | —H | —CN |
| B-XIA-64 | —CN | —H | —H | —OCH₃ |
| B-XIA-65 | —H | —CN | —H | —OCH₃ |
| B-XIA-66 | —H | —H | —CN | —OCH₃ |
| B-XIA-67 | —F | —SO₂CH₃ | —H | —H |
| B-XIA-68 | —H | —SO₂CH₃ | —F | —H |
| B-XIA-69 | —H | —SO₂CH₃ | —H | —F |
| B-XIA-70 | —F | —H | —SO₂CH₃ | —H |
| B-XIA-71 | —H | —F | —SO₂CH₃ | —H |
| B-XIA-72 | —H | —H | —SO₂CH₃ | —F |
| B-XIA-73 | —SO₂CH₃ | —F | —H | —H |
| B-XIA-74 | —SO₂CH₃ | —H | —F | —H |
| B-XIA-75 | —SO₂CH₃ | —H | —H | —F |
| B-XIA-76 | —F | —H | —H | —SO₂CH₃ |
| B-XIA-77 | —H | —F | —H | —SO₂CH₃ |
| B-XIA-78 | —H | —H | —F | —SO₂CH₃ |
| B-XIA-79 | —F | —F | —H | —H |
| B-XIA-80 | —H | —F | —F | —H |
| B-XIA-81 | —H | —F | —H | —F |
| B-XIA-82 | —F | —H | —F | —H |
| B-XIA-83 | —H | —H | —F | —F |
| B-XIA-84 | —F | —H | —H | —F |
| B-XIA-85 | —CN | —F | —H | —H |
| B-XIA-86 | —H | —F | —CN | —H |
| B-XIA-87 | —H | —F | —H | —CN |
| B-XIA-88 | —CN | —H | —F | —H |
| B-XIA-89 | —H | —CN | —F | —H |
| B-XIA-90 | —H | —H | —F | —CN |
| B-XIA-91 | —F | —CN | —H | —H |
| B-XIA-92 | —F | —H | —CN | —H |
| B-XIA-93 | —F | —H | —H | —CN |
| B-XIA-94 | —CN | —H | —H | —F |
| B-XIA-95 | —H | —CN | —H | —F |
| B-XIA-96 | —H | —H | —CN | —F |

Also provided is a compound, or a tautomer, stereoisomer, mixture of stereoisomers, isotopically enriched analog, or pharmaceutically acceptable salt thereof, represented by Formula B-XIB, as in Table B-XIB:

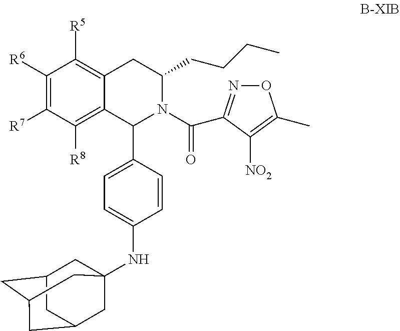

TABLE B-XIB

| No. | R⁵ | R⁶ | R⁷ | R⁸ |
|---|---|---|---|---|
| B-XIB-1 | —OCH₃ | —H | —H | —H |
| B-XIB-2 | —H | —OCH₃ | —H | —H |
| B-XIB-3 | —H | —H | —OCH₃ | —H |
| B-XIB-4 | —H | —H | —H | —OCH₃ |
| B-XIB-5 | —OCF₃ | —H | —H | —H |
| B-XIB-6 | —H | —OCF₃ | —H | —H |
| B-XIB-7 | —H | —H | —OCF₃ | —H |
| B-XIB-8 | —H | —H | —H | —OCF₃ |
| B-XIB-9 | —CH₃ | —H | —H | —H |
| B-XIB-10 | —H | —CH₃ | —H | —H |
| B-XIB-11 | —H | —H | —CH₃ | —H |
| B-XIB-12 | —H | —H | —H | —CH₃ |
| B-XIB-13 | —CF₃ | —H | —H | —H |
| B-XIB-14 | —H | —CF₃ | —H | —H |
| B-XIB-15 | —H | —H | —CF₃ | —H |
| B-XIB-16 | —H | —H | —H | —CF₃ |
| B-XIB-17 | —CN | —H | —H | —H |
| B-XIB-18 | —H | —CN | —H | —H |
| B-XIB-19 | —H | —H | —CN | —H |
| B-XIB-20 | —H | —H | —H | —CN |
| B-XIB-21 | —F | —H | —H | —H |
| B-XIB-22 | —H | —F | —H | —H |
| B-XIB-23 | —H | —H | —F | —H |
| B-XIB-24 | —H | —H | —H | —F |
| B-XIB-25 | —CF₂H | —H | —H | —H |
| B-XIB-26 | —H | —CF₂H | —H | —H |
| B-XIB-27 | —H | —H | —CF₂H | —H |
| B-XIB-28 | —H | —H | —H | —CF₂H |
| B-XIB-29 | —SO₂CH₃ | —H | —H | —H |
| B-XIB-30 | —H | —SO₂CH₃ | —H | —H |
| B-XIB-31 | —H | —H | —SO₂CH₃ | —H |
| B-XIB-32 | —H | —H | —H | —SO₂CH₃ |
| B-XIB-33 | —SO₂NHCH₃ | —H | —H | —H |
| B-XIB-34 | —H | —SO₂NHCH₃ | —H | —H |
| B-XIB-35 | —H | —H | —SO₂NHCH₃ | —H |
| B-XIB-36 | —H | —H | —H | —SO₂NHCH₃ |
| B-XIB-37 | —OCH₃ | —OCH₃ | —H | —H |
| B-XIB-38 | —H | —OCH₃ | —OCH₃ | —H |
| B-XIB-39 | —H | —OCH₃ | —H | —OCH₃ |
| B-XIB-40 | —OCH₃ | —H | —OCH₃ | —H |
| B-XIB-41 | —H | —H | —OCH₃ | —OCH₃ |
| B-XIB-42 | —OCH₃ | —H | —H | —OCH₃ |
| B-XIB-43 | —F | —OCH₃ | —H | —H |
| B-XIB-44 | —H | —OCH₃ | —F | —H |
| B-XIB-45 | —H | —OCH₃ | —H | —F |
| B-XIB-46 | —F | —H | —OCH₃ | —H |
| B-XIB-47 | —H | —F | —OCH₃ | —H |
| B-XIB-48 | —H | —H | —OCH₃ | —F |
| B-XIB-49 | —OCH₃ | —F | —H | —H |
| B-XIB-50 | —OCH₃ | —H | —F | —H |
| B-XIB-51 | —OCH₃ | —H | —H | —F |
| B-XIB-52 | —F | —H | —H | —OCH₃ |
| B-XIB-53 | —H | —F | —H | —OCH₃ |
| B-XIB-54 | —H | —H | —F | —OCH₃ |
| B-XIB-55 | —CN | —OCH₃ | —H | —H |
| B-XIB-56 | —H | —OCH₃ | —CN | —H |
| B-XIB-57 | —H | —OCH₃ | —H | —CN |
| B-XIB-58 | —CN | —H | —OCH₃ | —H |
| B-XIB-59 | —H | —CN | —OCH₃ | —H |
| B-XIB-60 | —H | —H | —OCH₃ | —CN |
| B-XIB-61 | —OCH₃ | —CN | —H | —H |
| B-XIB-62 | —OCH₃ | —H | —CN | —H |
| B-XIB-63 | —OCH₃ | —H | —H | —CN |
| B-XIB-64 | —CN | —H | —H | —OCH₃ |
| B-XIB-65 | —H | —CN | —H | —OCH₃ |
| B-XIB-66 | —H | —H | —CN | —OCH₃ |
| B-XIB-67 | —F | —SO₂CH₃ | —H | —H |
| B-XIB-68 | —H | —SO₂CH₃ | —F | —H |
| B-XIB-69 | —H | —SO₂CH₃ | —H | —F |
| B-XIB-70 | —F | —H | —SO₂CH₃ | —H |
| B-XIB-71 | —H | —F | —SO₂CH₃ | —H |
| B-XIB-72 | —H | —H | —SO₂CH₃ | —F |
| B-XIB-73 | —SO₂CH₃ | —F | —H | —H |
| B-XIB-74 | —SO₂CH₃ | —H | —F | —H |
| B-XIB-75 | —SO₂CH₃ | —H | —H | —F |
| B-XIB-76 | —F | —H | —H | —SO₂CH₃ |
| B-XIB-77 | —H | —F | —H | —SO₂CH₃ |
| B-XIB-78 | —H | —H | —F | —SO₂CH₃ |
| B-XIB-79 | —F | —F | —H | —H |
| B-XIB-80 | —H | —F | —F | —H |
| B-XIB-81 | —H | —F | —H | —F |
| B-XIB-82 | —F | —H | —F | —H |
| B-XIB-83 | —H | —H | —F | —F |
| B-XIB-84 | —F | —H | —H | —F |
| B-XIB-85 | —CN | —F | —H | —H |
| B-XIB-86 | —H | —F | —CN | —H |
| B-XIB-87 | —H | —F | —H | —CN |
| B-XIB-88 | —CN | —H | —F | —H |
| B-XIB-89 | —H | —CN | —F | —H |
| B-XIB-90 | —H | —H | —F | —CN |
| B-XIB-91 | —F | —CN | —H | —H |
| B-XIB-92 | —F | —H | —CN | —H |
| B-XIB-93 | —F | —H | —H | —CN |
| B-XIB-94 | —CN | —H | —H | —F |
| B-XIB-95 | —H | —CN | —H | —F |
| B-XIB-96 | —H | —H | —CN | —F |

### 3. Compositions and Methods of Use

Compounds for use in the compositions and methods disclosed herein include a compound of formula A-I, A-IA, A-IB, A-II, A-III, B-I, B-IA, B-IB, B-II, B-IIA, B-IIB, B-X, B-XIA, B-XIB, or a tautomer, stereoisomer, mixture of stereoisomers, isotopically enriched analog, or pharmaceutically acceptable salt thereof, a compound of Table A-1A, A-2A, A-IB, A-IC, A-2, A-3, B-1, B-2, B-3, B-4, B-5, B-6, B-7, B-8, B-9, B-10, B-XIA, or B-XIB, or a tautomer, stereoisomer, mixture of stereoisomers, isotopically enriched analog, or pharmaceutically acceptable salt thereof, or a compound of formula A-I:

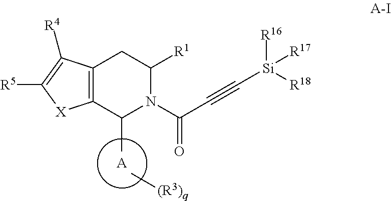

wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆ alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R is independently unsubstituted or substituted with one to three R¹⁰; or
when X is —NR²²—, —O—, or —S—, then R⁴ and R, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently unsubstituted or substituted with one to three R¹¹;
each R⁷ is independently hydrogen, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R or ring formed thereby is independently unsubstituted or substituted with one to three R¹¹;
each R⁸ is independently C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently unsubstituted or substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆ alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

In certain embodiments, the compounds described herein are used in a method of treating a disease, disorder, or condition, such as a cancer. In certain embodiments, the method of treating cancer comprises administering to a subject in need thereof a therapeutically effective amount any of the compounds described herein.

In certain embodiments, the compounds described herein are used in a method of treating a disease, disorder, or condition, such as a cancer. In certain embodiments, the method of treating a disease, disorder, or condition (e.g., cancer) comprises providing (e.g., administering) to a subject (e.g., patient) in need thereof a therapeutically effective amount any of the compounds described herein, or a salt, ester, tautomer, prodrug, zwitterionic form, or stereoisomer thereof, or a composition or pharmaceutical composition comprising the same.

In certain embodiments, the compounds provided herein are used in a method of modulating GPX4 in a cell, comprising contacting the cell with an effective amount of a compound or composition described herein to modulate GPX4 in the cell. In certain embodiments, the compounds are used in a method of inhibiting GPX4 in a cell, comprising contacting the cell with an effective amount of a compound or composition described herein to inhibit GPX4 in the cell. In certain embodiments, the cell is a cancer cell. In certain embodiments, a method comprising contacting a cell with a compound or composition provided herein is a component of a biochemical or cellular assay. In certain embodiments, the cell is disposed within the body of a mammal, such as a human subject. In certain embodiments, the method comprises administering an effective amount of a compound or composition described herein to a subject in need thereof.

In certain embodiments, the compounds are used in a method of inducing ferroptosis in a cell comprising contacting the cell with an effective amount of a compound or composition provided herein. In certain embodiments, the cell is disposed within the body of a mammal, such as a human subject. In certain embodiments, the method comprises administering an effective amount of a compound or composition described herein to a subject in need thereof.

In certain embodiments, provided is a method for treating a cancer in a patient in need thereof, comprising administering an effective amount of a compound or composition provided herein. In certain embodiments, provided is a method for treating a disease, disorder, or condition (e.g., cancer) in a subject in need thereof, comprising administering an effective amount of a compound or composition provided herein. In some embodiments, the subject has previously been diagnosed with or is otherwise known to have the disease, disorder, or condition, such as a cancer. In some embodiments, the subject has previously undergone treatment for the disease, disorder, or condition (e.g., cancer), or has previously entered remission for the disease, disorder, or condition (e.g., cancer).

In certain embodiments, the compounds are used in a method of treating cancer in a subject in need thereof, comprising administering to a subject having cancer a therapeutically effective amount of a compound (e.g., ferroptosis inducing compound) disclosed herein. Various cancers for treatment with the compounds include, but are not limited to, adrenocortical cancer, anal cancer, biliary cancer, bladder cancer, bone cancer, gliomas, astrocytoma, neuroblastoma, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, lymphoma, and soft tissue carcinomas. In certain embodiments, the compound is used to treat pancreatic cancer.

In certain embodiments, the cancer is renal cell carcinoma (RCC), pancreatic cancer, lung cancer, breast cancer, lymphoma, or prostate cancer. In certain embodiments, provided is a method for treating renal cell carcinoma (RCC) in a subject in need thereof, comprising administering an effective amount of a compound or composition provided herein. In certain embodiments, provided is a method for treating pancreatic cancer in a subject in need thereof, comprising administering an effective amount of a compound or composition provided herein. In certain embodiments, provided is a method for treating lung cancer in a subject in need thereof, comprising administering an effective amount of a compound or composition provided herein. In certain embodiments, provided is a method for treating breast cancer in a subject in need thereof, comprising administering an effective amount of a compound or composition provided herein. In certain embodiments, provided is a method for treating lymphoma in a subject in need thereof, comprising administering an effective amount of a compound or composition provided herein. In certain embodiments, provided is a method for treating prostate cancer in a subject in need thereof, comprising administering an effective amount of a compound or composition provided herein.

In certain embodiments, provided is a method for treating a malignant solid tumor in a subject in need thereof, comprising administering an effective amount of a compound or composition provided herein to the subject. In certain embodiments, the malignant solid tumor is a carcinoma. In certain embodiments, the malignant solid tumor is a lymphoma. In certain embodiments, the malignant solid tumor is a sarcoma.

In certain embodiments, the cancer for treatment with a compound or composition provided herein can be selected from, among others, adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, vaginal cancer, sarcoma, and soft tissue carcinomas. In certain embodiments, the cancer is renal cell carcinoma (RCC). In certain embodiments, the cancer is pancreatic cancer. In certain embodiments, the cancer is lung cancer. In certain embodiments, the cancer is breast cancer. In certain embodiments, the cancer is prostate cancer. In certain embodiments, the cancer is lymphoma.

In certain embodiments, the cancer for treatment with the compound is pancreatic cancer. In certain embodiments, the pancreatic cancer for treatment with the compounds is pancreatic adenocarcinoma or metastatic pancreatic cancer. In certain embodiments, the cancer for treatment with the compounds is stage I, stage II, stage III, or stage IV pancreatic adenocarcinoma.

In certain embodiments, the cancer for treatment with the compounds is lung cancer. In certain embodiments, the lung cancer for treatment with the compounds is small cell lung cancer or non-small cell lung cancer. In certain embodiments, the non-small cell lung cancer for treatment with the compounds is an adenocarcinoma, squamous cell carcinoma, or large cell carcinoma. In certain embodiments, the lung cancer for treatment with the compounds is metastatic lung cancer.

In certain embodiments, the cancer for treatment with the compounds is a hematologic cancer. In certain embodiments, the hematologic cancer is selected from acute lymphoblastic leukemia (ALL), acute myeloid leukemia (AML), lymphoma (e.g., Hodgkin's lymphoma, Non-Hodgkin's lymphoma, Burkitt's lymphoma, or diffuse large B-cell lymphoma), chronic lymphocytic leukemia (CLL), chronic myelogenous leukemia (CML), Hairy Cell chronic myelogenous leukemia (CML), and multiple myeloma.

In certain embodiments, the cancer for treatment with the compounds is a leukemia selected from acute lymphoblastic leukemia (ALL), acute myeloid leukemia (AML), chronic lymphocytic leukemia (CLL), chronic myelogenous leukemia (CML), Hairy Cell chronic myelogenous leukemia (CML), and multiple myeloma.

In certain embodiments, the cancer for treatment with the compound is a lymphoma selected from Hodgkin's lymphoma, non-Hodgkin's lymphoma, diffuse large B-cell lymphoma, and Burkitt's lymphoma. In some embodiments, the cancer is Hodgkin's lymphoma. In some embodiments, the cancer is non-Hodgkin's lymphoma. In some embodiments, the cancer is Burkitt's lymphoma. In some embodiments, the cancer is diffuse large B-cell lymphoma.

In certain embodiments, the cancer for treatment with the compound is a cancer characterized by mesenchymal features or mesenchymal phenotype. In some cancers, gain of mesenchymal features is associated with migratory (e.g., intravasation) and invasiveness of cancers. Mesenchymal features can include, among others, enhanced migratory capacity, invasiveness, elevated resistance to apoptosis, and increased production of extracellular matrix (ECM) components. In addition to these physiological characteristics, the mesenchymal features can include expression of certain biomarkers, including among others, E-cadherin, N-cadherin, integrins, FSP-1, α-SMA, vimentin, β-catenin, collagen I, collagen II, collagen III, collagen IV, fibronectin, laminin 5, SNAIL-1, SNAIL-2, Twist-1, Twist-2, and Lef-1. In certain embodiments, the cancer selected for treatment with the compounds herein include, among others, breast cancer, lung cancer, head and neck cancer, prostate cancer, and colon cancer. In certain embodiments, the mesenchymal features can be inherent to the cancer type or induced by or selected for by treatment of cancers with chemotherapy and/or radiation therapy.

In certain embodiments, the cancer for treatment with the compound is identified as having or determined to have an activating or oncogenic RAS activity. In certain embodiments, the RAS is K-RAS, H-RAS or N-RAS. In certain embodiments, the activating or oncogenic RAS is an activating or oncogenic RAS mutation.

In certain embodiments, the cancer selected for treatment with the compounds are determined to have or identified as having an activating or oncogenic RAS activity. In certain embodiments, the activating or oncogenic RAS activity is an activating or oncogenic RAS mutation. In certain embodiments, the activating or oncogenic RAS activity is an activating or oncogenic K-RAS activity, particularly an activating or oncogenic K-RAS mutation. In certain embodiments, the activating or oncogenic RAS activity is an activating or oncogenic N-RAS activity, particularly an activating or oncogenic N-RAS mutation. In certain embodiments, the activating or oncogenic RAS activity is an activating or oncogenic H-RAS activity, particularly an activating or oncogenic H-RAS mutation.

In certain embodiments, the compounds can be used to treat a cancer that is refractory to one or more other chemotherapeutic agents, particularly cytotoxic chemotherapeutic agents; or treat a cancer resistant to radiation treatment. In certain embodiments, the compounds are used to treat cancers that have developed tolerance to chemotherapeutic agents activating other cell death pathways, such as apoptosis, mitotic catastrophe, necrosis, senescence, and/or autophagy.

In certain embodiments, the cancer for treatment with the compounds is identified as being refractory or resistant to chemotherapy. In certain embodiments, the cancer is refractory or resistant to one or more of alkylating agents; anti-cancer antibiotic agents; antimetabolic agents (e.g., folate antagonists, purine analogs, pyrimidine analogs, etc.); topoisomerase inhibiting agents; anti-microtubule agents (e.g., taxanes, vinca alkaloids); hormonal agents (e.g., aromatase inhibitors); plant-derived agents and their synthetic derivatives; anti-angiogenic agents; differentiation inducing agents; cell growth arrest inducing agents; apoptosis inducing agents; cytotoxic agents; agents affecting cell bioenergetics i.e., affecting cellular ATP levels and molecules/activities regulating these levels; biologic agents, e.g., monoclonal antibodies; kinase inhibitors; and inhibitors of growth factors and their receptors.

In certain embodiments, the cancer for treatment with the compounds is a cancer identified as being refractory or resistant to one or more of afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelborae), vincristine, vinblastine, vinorelbine, and vindesine.

In certain embodiments, the cancer for treatment with the compound is identified as being refractory or resistant to one or more chemotherapeutics agents selected from cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and erlotinib.

In certain embodiments, the cancer for treatment with the compounds is a cancer resistant to ionizing radiation therapy. The radioresistance of the cancer can be inherent or as a result of radiation therapy. In certain embodiments, the cancers for treatment with the compounds is, among others, a radioresistant adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, or vaginal cancer. In certain embodiments, the cancer is pancreatic cancer, breast cancer, glioblastoma, advanced non-small-cell lung cancer, bladder cancer, sarcoma, lymphoma, or soft tissue carcinoma.

In certain embodiments, the compounds described herein are used in combination with one or more of other (e.g., second therapeutic agent) therapeutic treatments for cancer. In certain embodiments, the compounds can be used as monotherapy, or as further provided below, in a combination therapy with one or more therapeutic treatments, particularly in combination with one or more chemotherapeutic agents. In certain embodiments, the compounds are used in combination with a second therapeutic agent, where the compounds are used at levels that sensitizes the cancer or cancer cell to the second therapeutic agent, for example at levels of the compound that do not cause significant cell death. In certain embodiments, the compounds can be used in combination with radiation therapy, either to sensitize the cells to radiation therapy or as an adjunct to radiation therapy (e.g., at doses sufficient to activate cell death pathway).

In certain embodiments, a subject with cancer is treated with a combination of a compound described herein and radiation therapy. In certain embodiments, the method comprises administering to a subject with cancer a therapeutically effective amount of a compound of the disclosure, and adjunctively treating the subject with an effective amount of radiation therapy. In certain embodiments, the compound is administered to the subject in need thereof prior to, concurrently with, or subsequent to the treatment with radiation.

In certain embodiments, the method comprises administering an effective amount of a compound described herein to a subject with cancer to sensitize the cancer to radiation treatment, and administering a therapeutically effective amount of radiation therapy to treat the cancer. In certain embodiments, an effective amount of X-ray and gamma ray is administered to the subject. In certain embodiments, an effective amount of particle radiation is administered to the subject, where the particle radiation is selected from electron beam, proton beam, and neutron beam radiation. In certain embodiments, the radiation therapy is fractionated.

In certain embodiments, a subject with cancer is administered a therapeutically effective amount of a compound described herein, or a first pharmaceutical composition thereof, and adjunctively administered a therapeutically effective amount of a second chemotherapeutic agent, or a second pharmaceutical composition thereof.

In certain embodiments, the second chemotherapeutic agent is selected from an platinating agent; alkylating agent; anti-cancer antibiotic agent; antimetabolic agent (e.g., folate antagonists, purine analogs, pyrimidine analogs, etc.); topoisomerase I inhibiting agent; topoisomerase II inhibiting agent antimicrotubule agent (e.g., taxanes, vinca alkaloids); hormonal agent (e.g., aromatase inhibitors); plant-derived agent and synthetic derivatives thereof; anti-angiogenic agent; differentiation inducing agent; cell growth arrest inducing agent; apoptosis inducing agent; cytotoxic agent; agent affecting cell bioenergetics, i.e., affecting cellular ATP levels and molecules/activities regulating these levels; anti-cancer biologic agent (e.g., monoclonal antibodies); kinase inhibitors; and inhibitors of growth factors and their receptors.

In certain embodiments, the second chemotherapeutic agent is an angiogenesis inhibitor, such as, but not limited to, an inhibitor of soluble VEGFR-1, NRP-1, angiopoietin 2, TSP-1, TSP-2, angiostatin and related molecules, endostatin, vasostatin, calreticulin, platelet factor-4, TIMP, CDAI, Meth-1, Meth-2, IFN-α, IFN-β, IFN-γ, CXCL10, IL-4, IL-12, IL-18, prothrombin (kringle domain-2), antithrombin III fragment, prolactin, VEGI, SPARC, osteopontin, maspin, canstatin (a fragment of COL4A2), or proliferin-related protein. In certain embodiments, the angiogenesis inhibitor is bevacizumab (Avastin), itraconazole, carboxyamidotriazole, TNP-470 (an analog of fumagillin), CM101, IFN-α, IL-12, platelet factor-4, suramin, SU5416, thrombospondin, a VEGFR antagonist, an angiostatic steroid plus heparin, cartilage-derived angiogenesis inhibitory factor (CDAI), a matrix metalloproteinase inhibitor, angiostatin, endostatin, 2-methoxyestradiol, tecogalan, tetrathiomolybdate, thalidomide, thrombospondin, prolactin, a αVβ3 inhibitor, linomide, ramucirumab, tasquinimod, ranibizumab, sorafenib (Nexavar), sunitinib (Sutent), pazopanib (Votrient), or everolimus (Afinitor).

In certain embodiments, the second chemotherapeutic agent is a cyclin-dependent kinase (CDK) inhibitor (e.g., a CDK4/CDK6 inhibitor). Examples include, but are not limited to, palbociclib (Ibrance), Ribociclib (optionally further in combination with letrozole), abemaciclib (LY2835219; Verzenio), P1446A-05, and Trilaciclib (G1T28).

In certain embodiments, the second chemotherapeutic agent is a Bruton's tyrosine kinase (BTK) inhibitor, such as but not limited to, Ibrutinib (PCI-32765), acalabrutinib, ONO-4059 (GS-4059), spebrutinib (AVL-292, CC-292), BGB-3111, and HM71224.

In certain embodiments, the second chemotherapeutic agent is a BRAF inhibitor. Examples include, but are not limited to, BAY43-9006 (Sorafenib, Nexavar), PLX-4032 (Vemurafenib), GDC-0879, PLX-4720, dabrafenib and LGX818.

In certain embodiments, the second chemotherapeutic agent is a EGFR inhibitor. Examples include, but are not limited to, gefitinib, erlotinib, afatinib, brigatinib, icotinib, cetuximab, osimertinib, panitumumab, brigatinib, lapatinib, cimaVax-EGF, and veristrat.

In certain embodiments, the second chemotherapeutic agent is a human epidermal growth factor receptor 2 (HER2) inhibitor. Examples include, but are not limited to, trastuzumab, pertuzumab (optionally further in combination with trastuzumab), margetuximab, and NeuVax.

In certain embodiments, disclosed herein is a method of increasing a subject's responsiveness to an immunotherapeutic or immunogenic chemotherapeutic agent, the method comprising administering to the subject in need thereof an effective amount of a compound described herein and an effective amount of an immunotherapeutic agent and/or an immunogenic chemotherapeutic agent. In certain embodiments, the method further includes administering to the subject a lipoxygenase inhibitor. In certain embodiments, the subject has a tumor whose cellular microenvironment is stromal cell rich. In certain embodiments, the administration of compound described herein results in killing one or more stromal cells in the tumor cells' microenvironment. In certain embodiments, the administration of an effective amount of an immunotherapeutic agent and/or an immunogenic chemotherapeutic agent results in killing one or more tumor cells. Also provided herein is a combination comprising a compound described herein and an immunotherapeutic agent, lipoxygenase inhibitor, or immunogenic chemotherapeutic agent. In certain embodiments, the immunotherapeutic agent is selected from a CTLA4, PDL1 or PD1 inhibitor. In certain embodiments, the immunotherapeutic agent can be selected from CTLA4 inhibitor such as ipilimumab, a PD1 inhibitor such as pembrolizumab or nivolumab or a PDL1 inhibitor such as atezolizumab or durvalumab. In certain embodiments, the immunotherapeutic agent is pembrolizumab. In other embodiments, the immunogenic chemotherapeutic agent is a compound selected from anthracycline, doxorubicin, cyclophosphamide, paclitaxel, docetaxel, cisplatin, oxaliplatin or carboplatin. In certain embodiments, provided herein is a combination comprising a compound described herein and a lipoxygenase inhibitor. In certain embodiments, the lipoxygenase inhibitor is selected from PD147176 and/or ML351. In certain embodiments, the lipoxygenase inhibitor may be a 15-lipoxygenase inhibitor (see, e.g., Sadeghian et al., Expert Opinion on Therapeutic Patents, 2015, 26:1, 65-88).

In certain embodiments, the second chemotherapeutic agent is selected from an alkylating agent, including, but not limiting to, adozelesin, altretamine, bendamustine, bizelesin, busulfan, carboplatin, carboquone, carmofur, carmustine, chlorambucil, cisplatin, cyclophosphamide, dacarbazine, estramustine, etoglucid, fotemustine, hepsulfam, ifosfamide, improsulfan, irofulven, lomustine, mannosulfan, mechlorethamine, melphalan, mitobronitol, nedaplatin, nimustine, oxaliplatin, piposulfan, prednimustine, procarbazine, ranimustine, satraplatin, semustine, streptozocin, temozolomide, thiotepa, treosulfan, triaziquone, triethylenemelamine, triplatin tetranitrate, trofosphamide, and uramustine; an antibiotic, including, but not limiting to, aclarubicin, amrubicin, bleomycin, dactinomycin, daunorubicin, doxorubicin, elsamitrucin, epirubicin, idarubicin, menogaril, mitomycin, neocarzinostatin, pentostatin, pirarubicin, plicamycin, valrubicin, and zorubicin; an antimetabolite, including, but not limiting to, aminopterin, azacitidine, azathioprine, capecitabine, cladribine, clofarabine, cytarabine, decitabine, floxuridine, fludarabine, 5-fluorouracil, gemcitabine, hydroxyurea, mercaptopurine, methotrexate, nelarabine, pemetrexed, raltitrexed, tegafur-uracil, thioguanine, trimethoprim, trimetrexate, and vidarabine; an immunotherapy, an antibody therapy, including, but not limiting to, alemtuzumab, bevacizumab, cetuximab, galiximab, gemtuzumab, panitumumab, pertuzumab, rituximab, brentuximab, tositumomab, trastuzumab, 90 Y ibritumomab tiuxetan, ipilimumab, tremelimumab and anti-CTLA-4 antibodies; a hormone or hormone antagonist, including, but not limiting to, anastrozole, androgens, buserelin, diethylstilbestrol, exemestane, flutamide, fulvestrant, goserelin, idoxifene, letrozole, leuprolide, magestrol, raloxifene, tamoxifen, and toremifene; a taxane, including, but not limiting to, DJ-927, docetaxel, TPI 287, larotaxel, ortataxel, paclitaxel, DHA-paclitaxel, and tesetaxel; a retinoid, including, but not limiting to, alitretinoin, bexarotene, fenretinide, isotretinoin, and tretinoin; an alkaloid, including, but not limiting to, demecolcine, homoharringtonine, vinblastine, vincristine, vindesine, vinflunine, and vinorelbine; an antiangiogenic agent, including, but not limiting to, AE-941 (GW786034, Neovastat), ABT-510, 2-methoxyestradiol, lenalidomide, and thalidomide; a topoisomerase inhibitor, including, but not limiting to, amsacrine, belotecan, edotecarin, etoposide, etoposide phosphate, exatecan, irinotecan (also active metabolite SN-38 (7-ethyl-10-hydroxy-camptothecin)), lucanthone, mitoxantrone, pixantrone, rubitecan, teniposide, topotecan, and 9-aminocamptothecin; a kinase inhibitor, including, but not liming to, axitinib (AG 013736), dasatinib (BMS 354825), erlotinib, gefitinib, flavopiridol, imatinib mesylate, lapatinib, motesanib diphosphate (AMG 706), nilotinib (AMN107), seliciclib, sorafenib, sunitinib malate, AEE-788, BMS-599626, UCN-01 (7-hydroxystaurosporine), vemurafenib, dabrafenib, selumetinib, paradox breakers (such as PLX8394 or PLX7904), LGX818, BGB-283, pexidartinib (PLX3397) and vatalanib; a targeted signal transduction inhibitor including, but not limiting to bortezomib, geldanamycin, and rapamycin; a biological response modifier, including, but not limiting to, imiquimod, interferon-α, and interleukin-2; and other chemotherapeutics, including, but not limiting to 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguazone, oblimersen, sulindac, testolactone, tiazofurin, mTOR inhibitors (e.g. sirolimus, temsirolimus, everolimus, deforolimus, INK28, AZD8055, PI3K inhibitors (e.g. BEZ235, GDC-0941, XL147, XL765, BMK120), cyclin dependent kinase (CDK) inhibitors (e.g., a CDK4 inhibitor or a CDK6 inhibitor, such as Palbociclib (PD-0332991), Ribocyclib (LEE011), Abemaciclib (LY2835219), P1446A-05, Abemaciclib (LY2835219), Trilaciclib (G1T28), etc.), AKT inhibitors, Hsp90 inhibitors (e.g. geldanamycin, radicicol, tanespimycin), farnesyltransferase inhibitors (e.g. tipifarnib), Aromatase inhibitors (anastrozole letrozole exemestane); an MEK inhibitor including, but are not limited to, AS703026, AZD6244 (Selumetinib), AZD8330, BIX 02188, CI-1040 (PD184352), GSK1120212 (also known as trametinib or JTP-74057), cobimetinib, PD0325901, PD318088, PD98059, RDEA119 (BAY 869766), TAK-733 and U0126-EtOH; tyrosine kinase inhibitors, including, but are not limited to, AEE788, AG-1478 (Tyrphostin AG-1478), AG-490, Apatinib (YN968D1), AV-412, AV-951 (Tivozanib), Axitinib, AZD8931, BIBF1120 (Vargatef), BIBW2992 (Afatinib), BMS794833, BMS-599626, Brivanib (BMS-540215), Brivanib alaninate (BMS-582664), Cediranib (AZD2171), Chrysophanic acid (Chrysophanol), Crenolanib (CP-868569), CUDC-101, CYC116, Dovitinib Dilactic acid (TK1258 Dilactic acid), E7080, Erlotinib Hydrochloride (Tarceva, CP-358774, OSI-774, NSC-718781), Foretinib (GSK1363089, XL880), Gefitinib (ZD-1839 or Iressa), Imatinib (Gleevec), Imatinib Mesylate, Ki8751, KRN 633, Lapatinib (Tykerb), Linifanib (ABT-869), Masitinib (Masivet, AB1010), MGCD-265, Motesanib (AMG-706), MP-470, Mubritinib (TAK 165), Neratinib (HKI-272), NVP-BHG712, OSI-420 (Desmethyl Erlotinib, CP-473420), OSI-930, Pazopanib HCl, PD-153035 HCl, PD173074, Pelitinib (EKB-569), PF299804, Ponatinib (AP24534), PP121, RAF265 (CHIR-265), Raf265 derivative, Regorafenib (BAY 73-4506), Sorafenib Tosylate (Nexavar), Sunitinib Malate (Sutent), Telatinib (BAY 57-9352), TSU-68 (SU6668), Vandetanib (Zactima), Vatalanib dihydrochloride (PTK787), WZ3146, WZ4002, WZ8040, quizartinib, Cabozantinib, XL647, EGFR siRNA, FLT4 siRNA, KDR siRNA, Antidiabetic agents such as metformin, PPAR agonists (rosiglitazone, pioglitazone, bezafibrate, ciprofibrate, clofibrate, gemfibrozil, fenofibrate, indeglitazar), DPP4 inhibitors (sitagliptin, vildagliptin, saxagliptin, dutogliptin, gemigliptin, alogliptin) or an EGFR inhibitor, including, but not limited to, AEE-788, AP-26113, BIBW-2992 (Tovok), CI-1033, GW-572016, Iressa, LY2874455, RO-5323441, Tarceva (Erlotinib, OSI-774), CUDC-101 and WZ4002.

In certain embodiments, the second chemotherapeutic agent is selected from afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, idelalisib, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, olaparib, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelboraf), vincristine, vinblastine, vinorelbine, vindesine, and the like. In certain embodiments, the compounds herein are administered prior to, concurrently with, or subsequent to the treatment with the chemotherapeutic agent.

In certain embodiments, the method of treating a cancer comprises administering a therapeutically effective amount of a compound described herein and a therapeutically effective amount a biologic agent used to treat cancer. In certain embodiments, the biologic agent is selected from anti-BAFF (e.g., belimumab); anti-CCR4 (e.g., mogamulizumab); anti-CD19/CD3 (e.g., blinatumomab); anti-CD20 (e.g., obinutuzumab, rituximab, ibritumomab tiuxetan, ofatumumab, tositumomab); anti-CD22 (e.g., moxetumomab pasudotox); anti-CD30 (e.g., brentuximab vedotin); anti-CD33 (e.g., gemtuzumab); anti-CD37 (e.g., otlertuzumab); anti-CD38 (e.g., daratumumab); anti-CD52 (e.g., alemtuzumab); anti-CD56 (e.g., lorvotuzumab mertansine); anti-CD74 (e.g., milatuzumab); anti-CD105; anti-CD248 (TEM1) (e.g., ontuxizumab); anti-CTLA4 (e.g., tremelimumab, ipilimumab); anti-EGFL7 (e.g., parsatuzumab); anti-EGFR (HER1/ERBB1) (e.g., panitumumab, nimotuzumab, necitumumab, cetuximab, imgatuzumab, futuximab); anti-FZD7 (e.g., vantictumab); anti-HER2 (ERBB2/neu) (e.g., margetuximab, pertuzumab, ado-trastuzumab emtansine, trastuzumab); anti-HER3 (ERBB3); anti-HGF (e.g., rilotumumab, ficlatuzumab); anti-IGF-1R (e.g., ganitumab, figitumumab, cixutumumab, dalotuzumab); anti-IGF-2R; anti-KIR (e.g., lirilumab, onartuzumab); anti-MMP9; anti-PD-1 (e.g., nivolumab, pidilizumab, lambrolizumab); anti-PD-L1 (e.g. Atezolizumab); anti-PDGFRa (e.g., ramucirumab, tovetumab); anti-PD-L2; anti-PIGF (e.g., ziv-aflibercept); anti-RANKL (e.g., denosumab); anti-TNFRSF 9 (CD 137/4-1 BB) (e.g., urelumab); anti-TRAIL-RI/DR4, R2/D5 (e.g., dulanermin); anti-TRAIL-RI/D4 (e.g., mapatumumab); anti-TRAIL-R²/D5 (e.g., conatumumab, lexatumumab, apomab); anti-VEGFA (e.g., bevacizumab, ziv-aflibercept); anti-VEGFB (e.g., ziv-aflibercept); and anti-VEGFR2 (e.g., ramucirumab).

In certain embodiments, the pharmaceutical compositions of the compounds can be formulated by standard techniques using one or more physiologically acceptable carriers or excipients. Suitable pharmaceutical carriers are described herein and in Remington: The Science and Practice of Pharmacy, 21^{st} Ed. (2005). The therapeutic compounds and their physiologically acceptable salts, hydrates and solvates can be formulated for administration by any suitable route, including, among others, topically, nasally, orally, parenterally, rectally, or by inhalation. In certain embodiments, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets, capsules, and solutions can be administered orally, rectally, or vaginally.

For oral administration, a pharmaceutical composition can take the form of, for example, a tablet or a capsule prepared by various methods with a pharmaceutically acceptable excipient. Tablets and capsules comprising the active ingredient can be prepared together with excipients such as: (a) diluents or fillers, e.g., lactose, dextrose, sucrose, mannitol, sorbitol, cellulose (e.g., ethyl cellulose, microcrystalline cellulose), glycine, pectin, polyacrylates, and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; (c) binders, e.g., magnesium aluminum silicate, starch paste, gelatin, tragacanth, methylcellulose, sodium carboxymethylcellulose, polyvinylpyrrolidone, and/or hydroxypropyl methylcellulose; (d) disintegrants, e.g., starches (including potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners. The compositions are prepared using various methods including, for example, mixing, granulating, and coating methods.

In certain embodiments, the carrier is a cyclodextrin, such as to enhance solubility and/or bioavailability of the compounds herein. In certain embodiments, the cyclodextrin for use in the pharmaceutical compositions can be selected from α-cyclodextrin, β-cyclodextrin, γ-cyclodextrin, derivatives thereof, and combinations thereof. In certain embodiments, the cyclodextrin is selected from β-cyclodextrin, γ-cyclodextrin, derivatives thereof, and combinations thereof.

In certain embodiments, the compounds can be formulated with a cyclodextrin or derivative thereof selected from carboxyalkyl cyclodextrin, hydroxyalkyl cyclodextrin, sulfoalkylether cyclodextrin, and an alkyl cyclodextrin. In various embodiments, the alkyl group in the cyclodextrin is methyl, ethyl, propyl, butyl, or pentyl.

When used in a formulation with the compound of the present disclosure, the cyclodextrin can be present at about 0.1 w/v to about 30% w/v, about 0.1 w/v to about 20% w/v, about 0.5% w/v to about 10% w/v, or about 1% w/v to about 5% w/v. In certain embodiments, the cyclodextrin is present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, or about 30% w/v or more.

Tablets may be either film coated or enteric coated according to methods known in the art. Liquid preparations for oral administration can take the form of, for example, solutions, syrups, or suspensions, or they can be presented as a dry product for reconstitution with water or other suitable vehicle before use. Such liquid preparations can be prepared by various methods with pharmaceutically acceptable carriers and additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

The compounds can be formulated for parenteral administration, for example by bolus injection or continuous infusion. Formulations for injection can be presented in unit dosage form, for example, in ampoules or in multi-dose containers, with an optionally added preservative. Injectable compositions can be aqueous isotonic solutions or suspensions. In certain embodiments for parenteral administration, the compounds can be prepared with a surfactant, such as Cremaphor, or lipophilic solvents, such as triglycerides or liposomes. The compositions may be sterilized and/or contain adjuvants, such as preserving, stabilizing, wetting or emulsifying agents, solution promoters, salts for regulating the osmotic pressure and/or buffers. Alternatively, the compound can be in powder form for reconstitution with a suitable vehicle, for example, sterile pyrogen-free water, before use. In addition, they may also contain other therapeutically effective substances.

For administration by inhalation, the compound may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound with a carrier. Preferred carriers include absorbable pharmacologically acceptable solvents to assist passage through the skin of the subject. For example, transdermal devices are in the form of a bandage or patch comprising a backing member, a reservoir containing the compound optionally with carriers, optionally a rate controlling barrier to deliver the compound to the skin of the host at a controlled and predetermined rate over a prolonged period of time, and a means to secure the device to the skin. Matrix transdermal formulations may also be used.

Suitable formulations for topical application, e.g., to the skin and eyes, are preferably aqueous solutions, ointments, creams or gels well-known in the art. The formulations may contain solubilizers, stabilizers, tonicity enhancing agents, buffers and preservatives.

In certain embodiments, the compound can also be formulated as a rectal composition, for example, suppositories or retention enemas, for example, containing suppository bases, for example, cocoa butter or other glycerides, or gel forming agents, such as carbomers.

In certain embodiments, the compound can be formulated as a depot preparation. Such long-acting formulations can be administered by implantation (for example, subcutaneously or intramuscularly) or by intramuscular injection. The compound can be formulated with suitable polymeric or hydrophobic materials (for example as an emulsion in an acceptable oil), ion exchange resins, biodegradable polymers, or as sparingly soluble derivatives, for example, as a sparingly soluble salt.

The pharmaceutical compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

In certain embodiments, a pharmaceutical composition of the compound is administered to a subject, preferably a human, at a therapeutically effective dose to prevent, treat, or control a condition or disease as described herein. The pharmaceutical composition is administered to a subject in an amount sufficient to elicit an effective therapeutic response in the subject. An effective therapeutic response is a response that at least partially arrests or slows the symptoms or complications of the condition or disease. An amount adequate to accomplish this is defined as “therapeutically effective dose” or “therapeutically effective amount.” The dosage of compounds can take into consideration, among others, the species of warm-blooded animal (mammal), the body weight, age, condition being treated, the severity of the condition being treated, the form of administration, route of administration. The size of the dose also will be determined by the existence, nature, and extent of any adverse effects that accompany the administration of a particular therapeutic compound in a particular subject.

In certain embodiments, a suitable dosage of the compounds of the disclosure or a composition thereof is from about 1 nanogram per kilogram (ng/kg) to about 1000 milligrams per kilogram (mg/kg), from 0.01 mg/kg to 900 mg/kg, 0.1 mg/kg to 800 mg/kg, from about 1 mg/kg to about 700 mg/kg, from about 2 mg/kg to about 500 mg/kg, from about 3 mg/kg to about 400 mg/kg, 4 mg/kg to about 300 mg/kg, or from about 5 mg/kg to about 200 mg/kg, or a suitable range therein. In certain embodiments, the suitable dosages of the compound can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In certain embodiments, the dose of the compound can be administered once per day or divided into subdoses and administered in multiple doses, e.g., twice, three times, or four times per day.

In certain embodiments, the compounds can be administered with one or more of a second compound, sequentially or concurrently, either by the same route or by different routes of administration. When administered sequentially, the time between administrations is selected to benefit, among others, the therapeutic efficacy and/or safety of the combination treatment. In certain embodiments, the compounds herein can be administered first followed by a second compound, or alternatively, the second compound administered first followed by the compounds of the present disclosure. By way of example and not limitation, the time between administrations is about 1 hour (hr), about 2 hr, about 4 hr, about 6 hr, about 12 hr, about 16 hr or about 20 hr. In certain embodiments, the time between administrations is about 1, about 2, about 3, about 4, about 5, about 6, or about 7 more days. In certain embodiments, the time between administrations is about 1 week, 2 weeks, 3 weeks, or 4 weeks or more. In certain embodiments, the time between administrations is about 1 month or 2 months or more.

When administered concurrently, the compound can be administered separately at the same time as the second compound, by the same or different routes, or administered in a single composition by the same route. In certain embodiments, the amount and frequency of administration of the second compound can used standard dosages and standard administration frequencies used for the particular compound. See, e.g., Physicians' Desk Reference, 70th Ed., PDR Network, 2015; incorporated herein by reference.

In certain embodiments where the compounds of the present disclosure is administered in combination with a second compound, the dose of the second compound is administered at a therapeutically effective dose. In certain embodiments, a suitable dose can be from about 1 ng/kg to about 1000 mg/kg, from about 0.01 mg/kg to about 900 mg/kg, from about 0.1 mg/kg to about 800 mg/kg, from about 1 mg/kg to about 700 mg/kg, from about 2 mg/kg to about 500 mg/kg, from about 3 mg/kg to about 400 mg/kg, from about 4 mg/kg to about 300 mg/kg, or from about 5 mg/kg to about 200 mg/kg, or a suitable range therein. In certain embodiments, the suitable dosages of the second compound can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In certain embodiments, guidance for dosages of the second compound is provided in Physicians' Desk Reference, 70^{th}Ed, PDR Network (2015), incorporated herein by reference.

It to be understood that optimum dosages, toxicity, and therapeutic efficacy of such compounds may vary depending on the relative potency of individual compound and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. compounds or combinations thereof that exhibit large therapeutic indices are preferred. While certain agents that exhibit toxic side effects can be used, care should be used to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods disclosed herein, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

### 4. Methods of Preparation

The following examples are provided to further illustrate the methods of the present disclosure, and the compounds and compositions for use in the methods. The examples described are illustrative only and are not intended to limit the scope of the embodiments in any way. The disclosures of all articles and references mentioned in this application, including patents, are incorporated herein by reference in their entirety.

The compounds of the present disclosure can be synthesized in view of the guidance provided herein, incorporating known chemical reactions and related procedures such as separation and purification. Representative methods and procedures for preparation of the compounds in this disclosure are described below and in the Examples. Acronyms are abbreviations are used per convention which can be found in literature and scientific journals.

It is understood that the starting materials and reaction conditions may be varied, the sequence of the reactions altered, and additional steps employed to produce compounds encompassed by the present disclosure, as demonstrated by the following examples. General references for known chemical reactions useful for synthesizing the disclosed compounds are available (see, e.g., Smith and March, March's Advanced Organic Chemistry: Reactions, Mechanisms, and Structure, Fifth Edition, Wiley Interscience, 2001; or Carey and Sundberg, Advanced Organic Chemistry, Part B. Reaction and Synthesis; Fifth Edition, Springer, 2007; or Li, J. J. Name Reactions, A Collection of Detailed Mechanisms and Synthetic Applications; Fifth Edition, Springer, 2014).

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be readily determined by a skilled practitioner.

Additionally, protecting groups may be necessary to prevent certain functional groups from undergoing undesired reactions. Suitable protecting groups for various functional groups as well as suitable conditions for protecting and deprotecting particular functional groups are described in, for example, Wuts, P. G. M., Greene, T. W., & Greene, T. W. (2006) and Greene's protective groups in organic synthesis. Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons' 5th Edition, 2001), and Larock's Comprehensive Organic Transformations (VCH Publishers Inc., 1989).

### General Synthesis

In certain embodiments, compounds disclosed herein can be according to the general schemes shown below. For example, compounds disclosed herein (e.g., compounds of the Tables and of Formula A-I and B-I) can be prepared according to the general syntheses outlined below in Scheme 1, where suitable reagents can be purchased form commercial sources or synthesized via known methods or methods adapted from the examples provided herein. In Scheme 1, each of ring A, X, R¹, R², R³, R⁴, R⁵, R¹⁶, R¹⁷, R¹⁸, and q are independently as defined herein.

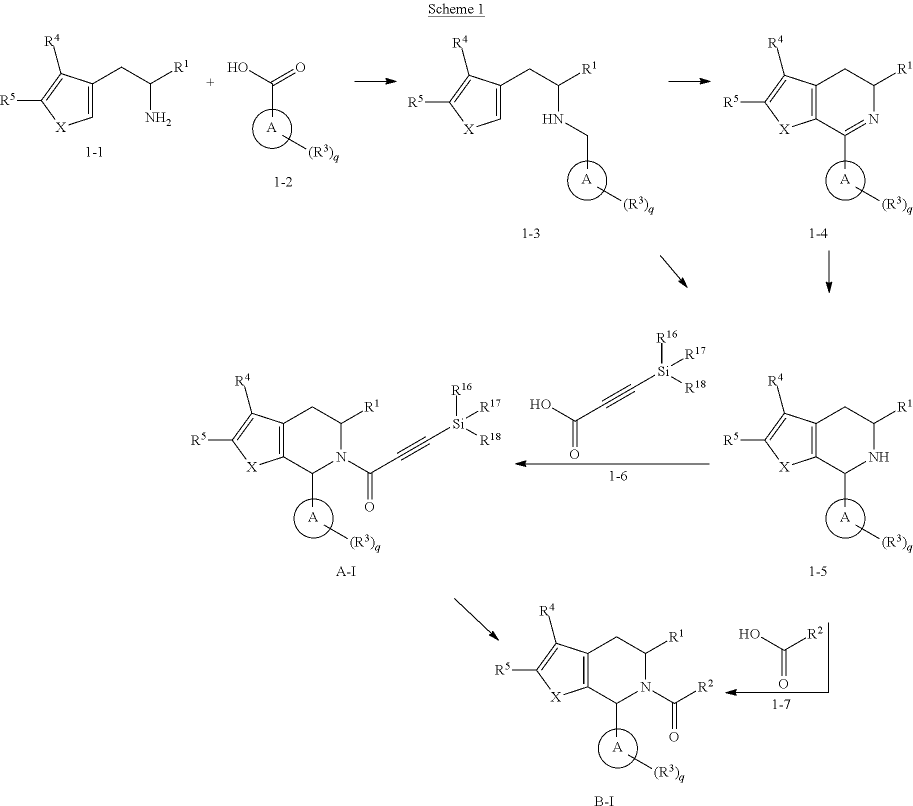

In Scheme 1, compound 1-3 can be provided by coupling amine 1-1 with acid 1-2 under standard amide bond forming reaction conditions. Cyclization of compound 1-3 to provide compound 1-5 can be achieved by first forming compound 1-4 followed by reduction using a hydride (e.g., NaBH₄, LiAlH₄, etc.). Alternatively, compound 1-5 can be provided directly from compound 1-3 under suitable conditions, such as an aprotic solvent in the presence of an acid catalyst. Compounds of Formula A-I can then be provided by coupling compound 1-5 with compound 1-6 under reaction conditions suitable to provide compounds of Formula A-I. Compounds of Formula B-I can then be provided by coupling compound 1-5 with compound 1-7 under reaction conditions suitable to provide compounds of Formula B-I. Upon each reaction completion, each of the intermediate or final compounds can be recovered, and optionally purified, by various techniques discernable by skill practitioners including, for example, neutralization, extraction, precipitation, chromatography, filtration, and the like.

Appropriate starting materials and reagents for use in Scheme 1 can be purchased or prepared by various techniques. As shown in Scheme 2, chiral or enantiomerically enriched starting materials can be provided for use in the method of Scheme 1 by converting a chiral or enantiomerically enriched amino alcohol to a oxathiazolidine dioxide 2-2. In Scheme 2, X, R¹, R⁴, and R⁵ are independently as defined herein, M is a metal halide (e.g., MgBr) and PG is a protecting group (e.g., Boc).

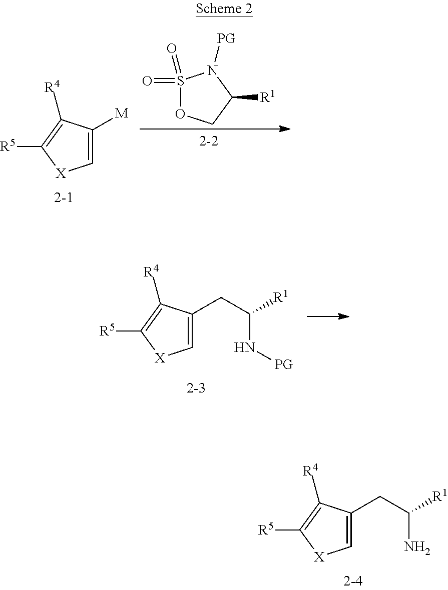

Referring to Scheme 2, compound 2-1 is coupled to compound 2-2 under standard coupling conditions to produce compound 2-3. The reaction is typically conducted in the presence of suitable catalyst (e.g., CuI) using suitable solvents/solvent mixtures. Deprotection of compound 2-3 provides compound 2-4. Upon reaction completion, each intermediate can be recovered by various techniques such as neutralization, extraction, precipitation, chromatography, filtration, and the like.

In some embodiments of the methods of Scheme 1 and Scheme 2, the various substituents on the starting compound (e.g., compound 1-1 and compound 1-2, (e.g., ring A, R¹, R³, R⁴, R⁵, etc.) are as defined herein (e.g., for Formula A-I or B-I). However, it should also be appreciated that chemical derivatization and/or functional group interconversion, can be used to further modify of any of the compounds of Scheme 1 or Scheme 2 in order to provide the various compounds disclosed herein (e.g., compounds of Formula A-I or B-I, or compounds of any of the Tables disclosed herein).

Other compounds of the disclosure can be synthesized using the synthetic routes above and adapting chemical synthetic procedures available to the skilled artisan. Exemplary methods of synthesis are provided in the Examples. It is to be understood that each of the procedures describing synthesis of exemplary compounds are part of the specification, and thus incorporated herein into the Detailed Description of this disclosure.

### Synthetic Examples

### Procedure 1: Synthesis of Compound A-6

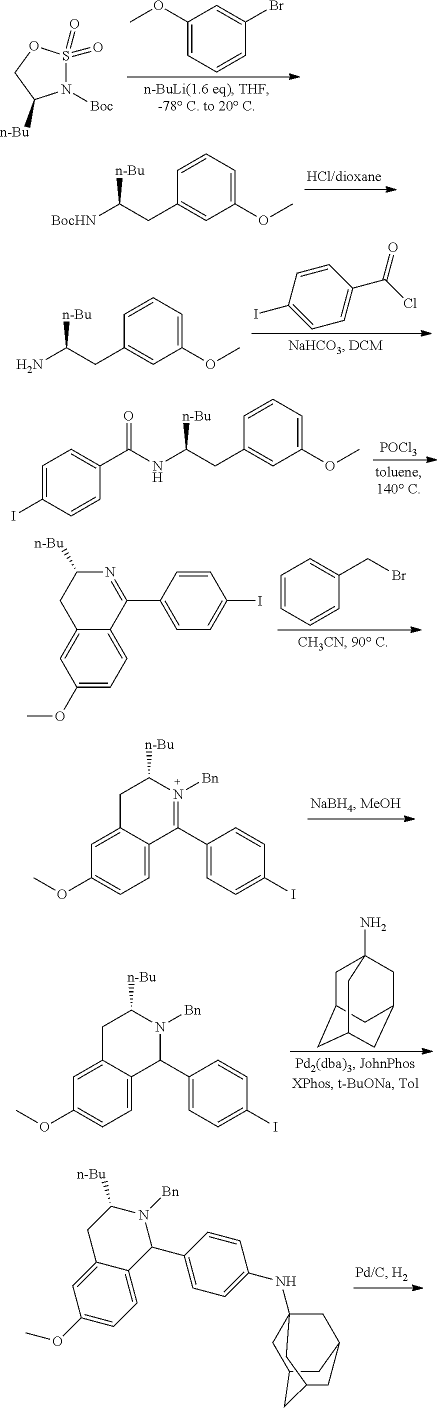

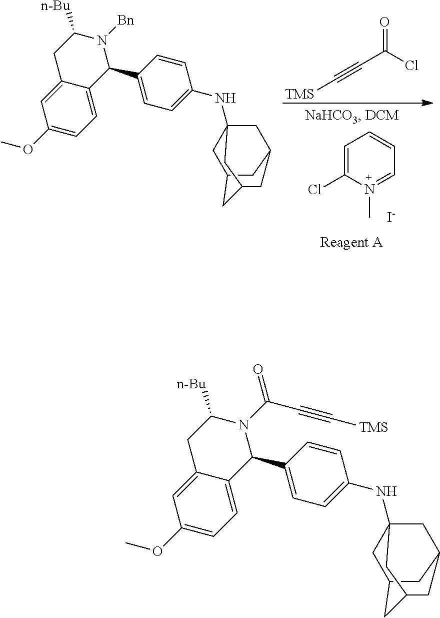

To a solution of 1-bromo-3-methoxy-benzene (10.04 g, 53.70 mmol, 6.79 mL, 1.5 eq) in THF (100 mL) was added n-BuLi (2.5 M, 22.91 mL, 1.6 eq) at −78° C. under N₂. The reaction was stirred at −78° C. under N₂ for 0.5 hr. Then tert-butyl (S)-4-butyl-1,2,3-oxathiazolidine-3-carboxylate 2,2-dioxide (10 g, 35.80 mmol, 1 eq) in THF (100 mL) was added dropwise at −78° C. The reaction was allowed to stir at 20° C. for 11.5 hr to give a yellow solution. TLC (PE/EtOAc=6:1) showed the reaction was completed. The reaction was quenched with sat. citric acid (200 mL) and extracted with EtOAc (100 mL*3). The organic layers were dried over Na₂SO₄, filtered and concentrated to give the crude product. The crude product was purified by flash column (SiO₂:PE/EtOAc=0% to 10%) to give tert-butyl (S)-(1-(3-methoxyphenyl)hexan-2-yl)carbamate. ¹H NMR (CDCl₃, 400 MHz): δ=7.19 (t, J=8.0 Hz, 1H), 6.80-6.69 (m, 3H), 4.40-4.30 (m, 1H), 3.87-3.74 (m, 4H), 2.80-2.62 (m, 2H), 1.55 (s, 2H), 1.43-1.28 (m, 13H), 0.89-0.83 (m, 3H).

To a solution of tert-butyl (S)-(1-(3-methoxyphenyl)hexan-2-yl)carbamate (5.35 g, 17.40 mmol, 1 eq) was added HCl/dioxane (4 M, 108.77 mL, 25 eq). The mixture was stirred at 20° C. for 12 hr to give a yellow suspension. TLC (PE/EtOAc=6:1) showed the reaction was completed. The mixture was concentrated to give (S)-1-(3-methoxyphenyl)hexan-2-amine HCl. The product was used in the next step without further purification. ¹H NMR (MeOD, 400 MHz): δ ppm 7.40-7.19 (m, 1H), 7.01-6.75 (m, 3H), 3.82 (s, 2H), 3.68 (s, 3H), 3.52-3.40 (m, 1H), 3.02-2.81 (m, 2H), 1.72-1.55 (m, 2H), 1.46-1.30 (m, 4H), 0.98-0.91 (m, 3H).

To a solution of (S)-1-(3-methoxyphenyl)hexan-2-amine (3.6 g, 17.37 mmol, 1 eq) in DCM (40 mL)/H₂O (20 mL) was added NaHCO₃ (17.51 g, 208.38 mmol, 8.10 mL, 12 eq) and 4-iodobenzoyl chloride (4.86 g, 18.23 mmol, 1.05 eq). The reaction was stirred at 20° C. for 1 hr to give a yellow solution. LCMS and TLC (eluting with PE/EtOAc=3/1) showed the reaction was completed. The reaction mixture was diluted with H₂O (10 mL) and concentrated to give the crude product. The crude product was purified by flash column (eluting with PE/EtOAc=0% to 20%) to give (S)-4-iodo-N-(1-(3-methoxyphenyl)hexan-2-yl)benzamide. ¹H NMR (CDCl₃, 400 MHz): δ=7.94-7.70 (m, 2H), 7.44-7.40 (m, 2H), 7.25-7.20 (m, 1H), 6.80-6.76 (m, 3H), 5.81-5.78 (m, 1H), 4.44-4.33 (m, 1H), 3.84 (s, 3H), 2.97-2.84 (m, 2H), 1.46-1.37 (m, 4H), 1.20-1.14 (m, 2H), 0.92-0.90 (m, 3H).

To a solution of (S)-4-iodo-N-(1-(3-methoxyphenyl)hexan-2-yl)benzamide (5.9 g, 13.49 mmol, 1 eq) in toluene (60 mL) was added POCl₃ (20.69 g, 134.91 mmol, 12.54 mL, 10 eq). The reaction was stirred at 140° C. for 1 hr to give a yellow solution. TLC (eluting with PE/EtOAc=3/1) showed the reaction was completed. The reaction mixture was poured into H₂O (100 mL) and extracted with EtOAc (100 mL*3). The organic layers were dried over Na₂SO₄, filtered and concentrated to give the crude product. The crude product was purified by flash column (eluting with PE/EtOAc=0% to 20%) to give (S)-3-butyl-1-(4-iodophenyl)-6-methoxy-3,4-dihydroisoquinoline. ¹H NMR (CDCl₃, 400 MHz): δ=7.78-7.76 (d, J=8.4 Hz, 2H), 7.36-7.34 (d, J=8.0 Hz, 2H), 7.19-7.14 (m, 1H), 6.81-6.76 (m, 1H), 6.75-6.72 (m, 1H), 3.85 (s, 3H), 3.55-3.45 (m, 1H), 2.84-2.60 (m, 1H), 2.65-2.59 (m, 1H), 1.95-1.85 (m, 1H), 1.66-1.36 (m, 6H), 0.98-0.94 (m, 3H).

To a solution of (S)-3-butyl-1-(4-iodophenyl)-6-methoxy-3,4-dihydroisoquinoline (3.6 g, 8.59 mmol, 1 eq) in CH₃CN (40 mL) was added bromomethyl benzene (7.34 g, 42.93 mmol, 5.10 mL, 5 eq). The reaction was stirred at 95° C. for 12 hr to give a yellow solution. LCMS showed the reaction was completed. The reaction mixture was concentrated to give the crude product. The crude product was triturated with PE (50 ml) and MTBE (50 mL) to give (S)-2-benzyl-3-butyl-1-(4-iodophenyl)-6-methoxy-3,4-dihydroisoquinolin-2-ium, which was used for the next step without further purification. ¹H NMR (MeOD, 400 MHz): δ=8.18-8.16 (m, 1H), 8.10-8.03 (m, 2H), 7.55-7.45 (m, 1H), 7.40-7.30 (m, 7H), 7.18-7.10 (m, 2H), 5.24-5.20 (m, 1H), 4.97-4.92 (m, 1H), 4.26-4.20 (m, 1H), 4.00 (m, 1H), 3.25-3.20 (m, 1H), 1.90-1.36 (m, 6H), 0.95-0.90 (m, 3H).

To a solution of (S)-2-benzyl-3-butyl-1-(4-iodophenyl)-6-methoxy-3,4-dihydroisoquinolin-2-ium (3.8 g, 7.44 mmol, 1 eq) in THF (40 mL) was added NaBH₄ (337.98 mg, 8.93 mmol, 1.2 eq) and MeOH (715.63 mg, 22.33 mmol, 903.80 μL, 3 eq) in THF (10 mL) at −78° C. The reaction was stirred at −78° C. for 0.5 hr to give a yellow solution. TLC (eluting with PE/EtOAc=3/1) showed the reaction was completed. The reaction mixture was quenched with sat. NH₄Cl (50 mL) and extracted with EtOAc (50 mL*3). The organic layers were dried over Na₂SO₄, filtered and concentrated to give the crude product. The crude product was purified by flash column (eluting with PE/EtOAc=0% to 15%) to give (3S)-2-benzyl-3-butyl-1-(4-iodophenyl)-6-methoxy-1,2,3,4-tetrahydroisoquinoline. ¹H NMR (CDCl₃, 400 MHz): δ=7.57-7.55 (m, 2H), 7.40-7.34 (m, 4H), 7.25-7.14 (m, 1H), 7.10-7.02 (m, 1H), 6.97-6.93 (m, 1H), 6.76-6.70 (m, 3H), 4.67-4.60 (m, 1H), 3.83 (s, 3H), 3.80-3.68 (m, 1H), 3.18-2.70 (m, 3H), 1.40-1.30 (m, 6H), 0.89-0.83 (m, 3H).

To a solution of (3S)-2-benzyl-3-butyl-1-(4-iodophenyl)-6-methoxy-1,2,3,4-tetrahydroisoquinoline (2.2 g, 4.30 mmol, 1 eq) in t-BuOH (20 mL) was added adamantan-1-amine (1.95 g, 12.90 mmol, 3 eq), XPhos (205.06 mg, 430.16 μmol, 0.1 eq), Pd₂(dba)₃ (196.95 mg, 215.08 μmol, 0.05 eq), JohnPhos (128.36 mg, 430.16 μmol, 0.1 eq) and t-BuONa (1.24 g, 12.90 mmol, 3 eq) under N₂. The reaction was stirred at 120° C. for 12 hr to give a black suspension. TLC (eluting with PE/EtOAc=6/1) showed the reaction was completed. The reaction mixture was filtered on celite and washed with EtOAc (50 mL). The filtrate was concentrated to give the crude product. The crude product was purified by flash column (eluting with PE/EtOAc=0% to 10%) to give (1S,3R)—N-(4-((3S)-2-benzyl-3-butyl-6-methoxy-1,2,3,4-tetrahydroisoquinolin-1-yl)phenyl)adamantan-1-amine. ¹H NMR (CDCl₃, 400 MHz): δ=7.42-7.28 (m, 4H), 7.27-7.22 (m, 1H), 7.15-6.94 (m, 2H), 6.75-6.66 (m, 5H), 4.66-4.59 (m, 1H), 3.80 (s, 3H), 3.78-3.40 (m, 3H), 2.90-2.16 (m, 3H), 2.90-2.10 (brs, 3H), 1.87-1.84 (m, 6H), 1.68-1.54 (m, 6H), 1.50-1.29 (m, 6H), 0.92-0.86 (m, 3H).

To a solution of (1S,3R)—N-(4-((3S)-2-benzyl-3-butyl-6-methoxy-1,2,3,4-tetrahydroisoquinolin-1-yl)phenyl)adamantan-1-amine (1.1 g, 2.06 mmol, 1 eq) in MeOH (20 mL) was added Pd(OH)₂ (224.49 mg, 15.99 μmol, 7.77 e-3 eq) under N₂. The suspension was degassed under vacuum and purged with H₂ several times. The mixture was stirred under H₂ (4.15 mg, 2.06 mmol, 1 eq) (15 psi) at 20° C. for 12 hr to give a black suspension. TLC (eluting with PE/EtOAc=2/1) showed the reaction was completed. The reaction mixture was filtered on celite and washed with MeOH (50 mL). The filtrate was concentrated to give the crude product. The crude product was purified by prep-TLC (eluting with PE/EtOAc=3/1) to give N-[4-[(1S,3S)-3-butyl-6-methoxy-1,2,3,4-tetrahydroisoquinolin-1-yl]phenyl]adamantan-1-amine. ¹H NMR (CDCl₃, 400 MHz): δ=6.94-6.84 (m, 3H), 6.70-6.50 (m, 4H), 5.11 (s, 1H), 3.80 (s, 3H), 2.98-2.94 (m, 1H), 2.87-2.80 (m, 1H), 2.58-2.46 (m, 1H), 2.09 (brs, 3H), 1.86-1.83 (m, 6H), 1.70-1.64 (m, 6H), 1.42-1.40 (m, 2H), 1.39-1.20 (m, 4H), 0.86-0.82 (m, 3H).

To a solution of 3-trimethylsilylprop-2-ynoic acid (463.80 mg, 3.26 mmol, 1 eq) in DCM (20 mL) was added 2-chloro-1-methylpyridin-1-ium iodide (833.13 mg, 3.26 mmol, 1 eq). The reaction was stirred at 20° C. for 0.5 hr. Then the mixture was added dropwise the a solution of N-[4-[(1S,3S)-3-butyl-6-methoxy-1,2,3,4-tetrahydroisoquinolin-1-yl]phenyl]adamantan-1-amine (1.45 g, 3.26 mmol, 1 eq) and Et₃N (329.98 mg, 3.26 mmol, 453.89 μL, 1 eq) in DCM (20 mL) at 0° C. The reaction was stirred at 0° C. for 1 hr to give a yellow solution. LCMS and TLC (eluting with PE/EtOAc=2/1) showed the reaction was completed. The reaction mixture was quenched with H₂O (30 mL) and extracted with DCM (30 mL*3). The organic layers were washed with HCl (0.5N, 20 mL), then washed with sat. NaHCO₃ (30 mL). The organic layers were then dried over Na₂SO₄, filtered and concentrated to give the crude product. The crude product was purified by flash column (eluting with PE/EtOAc=0% to 30%) to give 1-((1S,3S)-1-(4-(((1s,3R)-adamantan-1-yl)amino)phenyl)-3-butyl-6-methoxy-3,4-dihydroisoquinolin-2(1H)-yl)-3-(trimethylsilyl)prop-2-yn-1-one. LC-MS (m/z): 569.3[M+H]⁺. ¹H NMR (400 MHz, DMSO-d6) δ=7.33-7.10 (m, 1H), 6.70-6.58 (m, 4H), 6.56-6.36 (m, 2H), 5.98-5.70 (m, 1H), 4.70-4.50 (m, 1H), 4.46-4.10 (m, 2H), 3.60-3.10 (m, 3H), 2.84-2.44 (m, 2H), 1.87 (brs, 3H), 1.60 (brs, 6H), 1.40 (brs, 6H), 1.10-0.86 (m, 6H), 0.64-0.52 (m, 3H), 0.05-0.01 (m, 9H).

### Procedure 2: Synthesis of Compound A-7

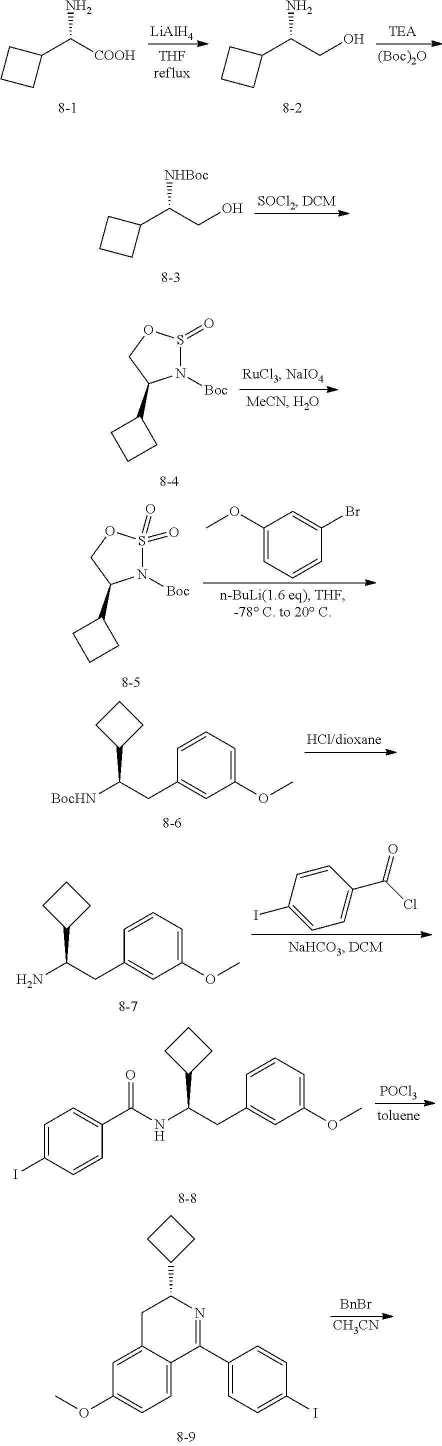

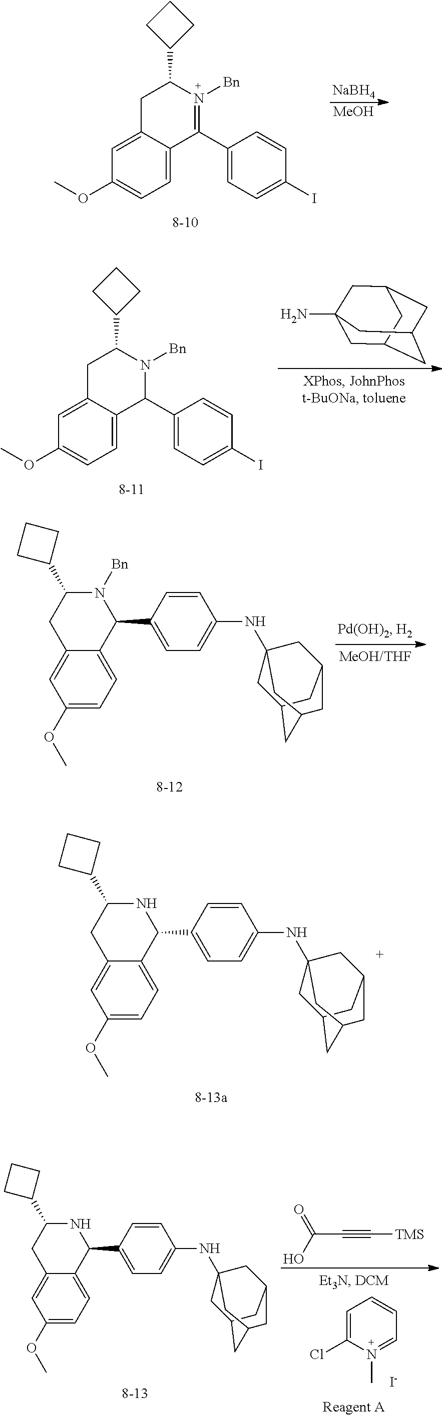

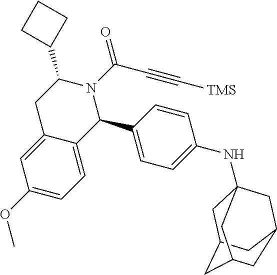 [SMILES: COc1ccc2c(c1)C[C@H](C1CCC1)N(C(=O)C#C[Si](C)(C)C)[C@H]2c1ccc(NC23CC4CC(CC(C4)C2)C3)cc1]

To a solution of LiAlH₄ (1.47 g, 38.71 mmol, 2.5 eq) in THF (20 mL) was added (2S)-2-amino-2-cyclobutyl-acetic acid (2 g, 15.49 mmol, 1 eq) at 0° C. The reaction was stirred at 50° C. for 4 hr to give a yellow suspension. NMR showed the reaction was completed. The reaction mixture was quenched with H₂O (1.47 mL), NaOH (15%, 1.47 mL) and H₂O (4.41 mL). The mixture was diluted with THF (30 mL). The mixture was filtered on celite and washed with THF (50 ml). The filtrate was concentrated to give intermediate 8-2, which was used for the next step without further purification. ¹H NMR (CDCl₃, 400 MHz): δ=3.60-3.50 (m, 1H), 3.25-3.15 (m, 1H), 2.80-2.74 (m, 1H), 2.25-1.90 (m, 4H), 1.80-1.66 (m, 3H).

To a solution of intermediate 8-2 (1.5 g, 13.02 mmol, 1 eq) in DCM (10 mL) was added Et₃N (1.98 g, 19.54 mmol, 2.72 mL, 1.5 eq) at 0° C. Then Boc₂O (2.84 g, 13.02 mmol, 2.99 mL, 1 eq) in DCM (10 mL) was added dropwise at 0° C. The reaction was allowed to stir at 20° C. for 12 hr to give a yellow solution. TLC (eluting with PE/EtOAc=2/1) showed the reaction was completed. The reaction was quenched with H₂O (30 mL) and extracted with DCM (30 mL*3). The organic layers were dried over Na₂SO₄ and concentrated to give the crude product. The crude product was purified by flash column (eluting with PE/EtOAc=0% to 30%) to give intermediate 8-3. ¹H NMR (CDCl₃, 400 MHz): δ=4.60 (brs, 1H), 3.82-3.41 (m, 3H), 2.42 (brs, 1H), 2.07-1.80 (m, 6H), 1.47 (s, 9H).

Imidazole (3.71 g, 54.44 mmol, 5.86 eq) was dissolved in DCM (60 mL) and cooled to 0° C. SOCl₂ (1.92 g, 16.16 mmol, 1.17 mL, 1.74 eq) dissolved in DCM (12 mL) was added dropwise and the resulting suspension was allowed to warm to 20° C. Stirring was continued for 1 h at 20° C., and then the mixture was cooled to −78° C. A solution of intermediate 8-3 (2 g, 9.29 mmol, 1 eq) in DCM (60 mL) was added over a period of 1 h. The resulting mixture was allowed to warm to 20° C. and stirred for 12 hr to give a yellow suspension. TLC eluting with PE/EtOAc=2/1 showed the reaction was completed. The reaction mixture was filtered and washed with DCM (40 mL). The organic layers were dried over Na₂SO₄ and concentrated to give intermediate 8-4, which was used for the next step without further purification. ¹H NMR (CDCl₃, 400 MHz): δ=4.88-4.02 (m, 3H), 2.80-2.48 (m, 1H), 2.00-1.60 (m, 6H), 1.45 (s, 9H).

To a solution of intermediate 8-4 (2.5 g, 9.57 mmol, 1 eq) in CH₃CN (20 mL)/H₂O (10 mL) was added RuCl₃·H₂O (10.78 mg, 47.83 μmol, 0.005 eq)/NaIO₄ (2.25 g, 10.52 mmol, 583.09 μL, 1.1 eq) at 0° C. The reaction was allowed to stir 20° C. for 12 hr to give a yellow solution. TLC (eluting with PE/EtOAc=3/1) showed the reaction was completed. The reaction mixture was filtered and washed with EtOAc (30 mL*3). The filtrate was washed with H₂O (30 mL). The organic layer was dried over Na₂SO₄ and concentrated to give intermediate 8-5, which was used for the next step without further purification. ¹H NMR (CDCl₃, 400 MHz): δ=4.56-4.50 (m, 1H), 4.25-4.16 (m, 2H), 2.82-2.69 (m, 1H), 2.82-2.69 (m, 1H), 2.00-1.70 (m, 6H), 1.48 (s, 9H).

To a solution of intermediate 8-5 (5 g, 18.03 mmol, 1 eq) in THF (50 mL) was added dropwise n-BuLi (2.5 M, 11.54 mL, 1.6 eq) at −78° C. The reaction was stirred at −78° C. for 0.5 hr. Then 1-bromo-3-methoxy-benzene (5.06 g, 27.04 mmol, 3.42 mL, 1.5 eq) in THF (30 mL) was added dropwise at −78° C. The reaction was allowed to stir at 20° C. for 11.5 hr to give a yellow solution. LCMS and TLC (eluting with PE/EtOAc=5/1) showed the reaction was completed. The reaction mixture was quenched with sat. citric acid (20 mL) and extracted with EtOAc (30 mL*3). The organic layers were dried over Na₂SO₄ and concentrated to give the crude product. The crude product was purified by flash column (eluting with PE/EtOAc=0% to 20%) to give intermediate 8-6.

Intermediate 8-6 (1.5 g, 4.91 mmol, 1 eq) was dissolved in HCl/dioxane (4 M, 20 mL, 16.29 eq). The reaction was stirred at 20° C. for 12 hr to give a yellow solution. NMR showed the reaction was completed. The reaction mixture was concentrated to give intermediate 8-7, which was used for the next step without further purification. ¹H NMR (MeOD, 400 MHz): δ ppm 7.31-7.30 (t, J=4.8 Hz, 1H), 6.90-6.82 (m, 3H), 3.82 (s, 3H), 3.44-3.30 (m, 1H), 2.95-2.86 (m, 2H), 2.79-2.74 (m, 1H), 2.01-1.81 (m, 6H).

To a solution of intermediate 8-7 (205 mg, 847.96 μmol, 1 eq, HCl) in DCM (10 mL)/H₂O (3 mL) was added NaHCO₃ (71.23 mg, 847.96 μmol, 32.98 μL, 1 eq) and 4-iodobenzoyl chloride (239.51 mg, 898.84 μmol, 1.06 eq). The reaction was stirred at 25° C. for 12 hr to give a yellow solution. LCMS and TLC (eluting with PE/EtOAc=5/1) showed the reaction was completed. The reaction mixture was diluted with H₂O (20 mL) and extracted with DCM (20 mL*3). The organic layers were dried over Na₂SO₄ and concentrated to give the crude product. The crude product was purified by flash column (eluting with PE/EtOAc=0% to 30%) to give intermediate 8-8. ¹H NMR (CDCl₂, 400 MHz): δ ppm 7.79-7.77 (d, J=6.8 Hz, 1H), 7.42-7.40 (d, J=8.4 Hz, 1H), 7.25-7.18 (m, 1H), 6.80-6.70 (m, 3H), 5.70-6.68 (d, J=9.2 Hz, 3H), 4.45-4.30 (m, 1H), 3.79 (s, 3H), 2.96-2.89 (m, 1H), 2.78-2.70 (m, 1H), 2.45-2.40 (m, 1H), 2.01-1.81 (m, 6H).

To a solution of intermediate 8-8 (1.4 g, 3.22 mmol, 1 eq) in toluene (10 mL) was added POCl₃ (4.93 g, 32.16 mmol, 2.99 mL, 10 eq). The reaction was stirred at 140° C. for 1 hr to give a yellow solution. TLC (eluting with PE/EtOAc=5/1) showed the reaction was completed. The reaction mixture was poured into H₂O (50 ml) and extracted with EtOAc (50 mL*3). The organic layers were dried over Na₂SO₄ and concentrated to give intermediate 8-9, which was used for the next step without further purification.

To a solution of 8-9 (1.34 g, 3.21 mmol, 1 eq) in CH₃CN (20 mL) was added BnBr (1.65 g, 9.63 mmol, 1.14 mL, 3 eq). The reaction was stirred at 85° C. for 12 hr to give a yellow solution. LCMS showed the reaction was completed. The reaction mixture was concentrated to give intermediate 8-10, which was used for the next step without further purification.

To a solution of 8-10 (1.63 g, 3.21 mmol, 1 eq) in MeOH (20 mL) was added NaBH₄ (363.88 mg, 9.62 mmol, 3 eq) at −78° C. for 1 h to give a solution. TLC (eluting with PE/EtOAc=3/1) showed the reaction was completed. The reaction mixture was quenched with sat. NH₄Cl (20 mL) and extracted with EtOAc (20 mL*3). The organic layers were dried over Na₂SO₄ and concentrated to give the crude product. The crude product was purified by flash column (eluting with PE/EtOAc=0% to 20%) to give intermediate 8-11. NMR showed a mixture of cis- and trans-isomers.

To a solution of intermediate 8-11 (500 mg, 981.51 μmol, 1 eq) in toluene (8 mL) was added adamantan-1-amine (445.35 mg, 2.94 mmol, 3 eq) and t-BuONa (282.98 mg, 2.94 mmol, 3 eq) under N₂. Then XPhos (93.58 mg, 196.30 μmol, 0.2 eq), JohnPhos (117.15 mg, 392.60 μmol, 0.4 eq) and Pd₂(dba)₃ (89.88 mg, 98.15 μmol, 0.1 eq) were added under N₂. The reaction was stirred at 125° C. for 12 hr to give a black suspension. LCMS and TLC (eluting with PE/EtOAc=5/1) showed the reaction was completed. The reaction mixture was concentrated to give the crude product. The crude product was purified by flash column (eluting with PE/EtOAc=0% to 30%) to give intermediate 8-12. ¹H NMR (CDCl₃, 400 MHz): δ ppm 7.32-7.25 (m, 4H), 7.24-7.18 (m, 1H), 6.86-6.60 (m, 7H), 4.58-4.47 (m, 1H), 3.76-3.50 (m, 5H), 3.34-3.20 (m, 1H), 2.90-2.70 (m, 1H), 2.64-2.40 (m, 3H), 2.01-1.85 (m, 5H), 1.78-1.60 (m, 7H), 1.54-1.50 (m, 8H).

To a solution of intermediate 8-12 (320 mg, 600.65 μmol, 1 eq) in EtOH (20 mL) was added Pd/C (100 mg, 50% purity) and cone. HCl (110.00 mg, 1.32 mmol, 0.1 mL, 2.19 eq) under N₂. The suspension was degassed under vacuum and purged with H₂ several times. The mixture was stirred under H₂ (1.21 mg, 600.65 μmol, 1 eq) (15 psi) at 25° C. for 2 hr. LCMS and TLC (eluting with PE/EtOAc=1/1) showed the reaction was completed. The reaction mixture was filtered and washed with MeOH (20 mL). The filtrate was concentrated to give the crude product. The crude product was purified by flash column (eluting with PE/EtOAc=0% to 50%) to give intermediate 8-13 and intermediate 8-13a. ¹H NMR (CDCl₃, 400 MHz): δ ppm 6.86-6.74 (m, 3H), 6.66-6.56 (m, 5H), 5.02 (s, 1H), 3.76-3.72 (s, 3H), 2.83-2.70 (m, 2H), 2.45-2.23 (m, 2H), 2.05-1.94 (m, 3H), 1.92-1.85 (m, 3H), 1.80-1.70 (m, 15H).

To a solution of 3-trimethylsilylprop-2-ynoic acid (37.59 mg, 264.33 μmol, 0.9 eq) in DCM (5 mL) was added 2-chloro-1-methylpyridin-1-ium iodide (67.53 mg, 264.33 μmol, 0.9 eq). The reaction was stirred at 25° C. for 0.5 hr. Then the mixture was added dropwise the a solution of intermediate 8-13 (130 mg, 293.70 μmol, 1 eq) and Et₃N (29.72 mg, 293.70 μmol, 40.88 μL, 1 eq) in DCM (5 mL) at 0° C. The reaction was stirred at 0° C. for 0.5 hr to give a yellow solution. TLC (eluting with PE/EtOAc=2/1) showed the reaction was completed. The reaction mixture was quenched with H₂O (0.5N, 10 mL) and extracted with DCM (20 mL*3). The organic layers were washed with sat. NaHCO₃ (15 mL). The organic layers were dried over Na₂SO₄ and concentrated to give the crude product. The crude product was purified by flash column (eluting with PE/EtOAc=0% to 30%) to give 1-((1S,3R)-1-(4-(((3R,5R,7R)-adamantan-1-yl)amino)phenyl)-3-cyclobutyl-6-methoxy-3,4-dihydroisoquinolin-2(1H)-yl)-3-(trimethylsilyl)prop-2-yn-1-one. LC-MS (m/z): 567.3[M+H]⁺. ¹H NMR (400 MHz, CDCl₃) δ=7.19-7.02 (m, 1H), 6.88-6.84 (m, 2H), 6.71-6.48 (m, 4H), 6.15-5.92 (m, 1H), 4.65-4.48 (m, 1H), 3.70-3.67 (m, 3H), 3.14-3.05 (m, 2H), 2.82-2.52 (m, 1H), 2.15-1.80 (m, 5H), 1.71-1.65 (m, 8H), 1.63-1.58 (m, 8H), 0.18-0.08 (m, 9H).

### Procedure 3: Synthesis of Compound A-8

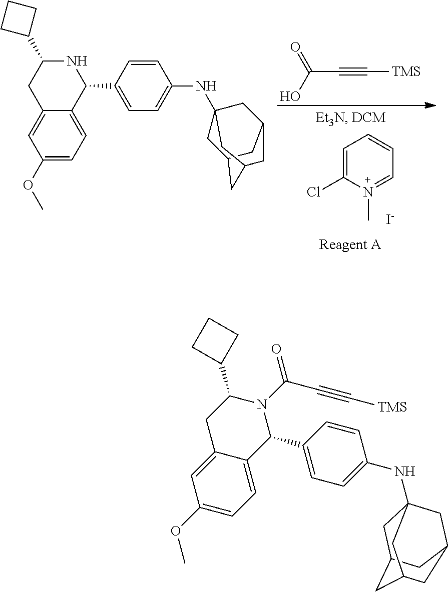

To a solution of 3-trimethylsilylprop-2-ynoic acid (22.49 mg, 158.14 μmol, 1 eq) in DCM (3 mL) was added 2-chloro-1-methyl-pyridin-1-ium iodide (40.40 mg, 158.14 μmol, 1 eq). The mixture was stirred at 25° C. for 1 hr to give a yellow suspension. The resulting mixture was added dropwise to a solution of (1s,3S)—N-(4-((1R,3R)-3-cyclobutyl-6-methoxy-1,2,3,4-tetrahydroisoquinolin-1-yl)phenyl)adamantan-1-amine (70 mg, 158.14 μmol, 1 eq) and TEA (16.00 mg, 158.14 μmol, 22.01 μL, 1 eq) in DCM (5 mL) at 0° C. The mixture was stirred at 0° C. for 2 hr to give a yellow solution. LCMS and TLC (twice PE:EtOAc=1:1, then PE:EtOAc=3:1) showed the reaction was completed. The reaction was quenched with H₂O (8 mL) and extracted with DCM (8 mL*3). The organic layers were washed with HCl (1N, 10 mL). The organic layer was separated. The organic layers were washed with sat. NaHCO₃ (10 mL). The organic layer was separated and dried over sodium sulfate, filtered and concentrated under vacuum. The residue was purified by column chromatography (SiO₂, Petroleum ether/Ethyl acetate=100/0 to 80/20) to give 1-((1R,3R)-1-(4-(((3S,5S,7S)-adamantan-1-yl)amino)phenyl)-3-cyclobutyl-6-methoxy-3,4-dihydroisoquinolin-2(1H)-yl)-3-(trimethylsilyl)prop-2-yn-1-one. LC-MS (m/z): 567.2 [M+H]⁺. ¹H NMR (400 MHz, CDCl₃) δ ppm 7.13-6.97 (m, 3H) 6.81-6.60 (m, 5H) 4.85-4.72 (m, 2H) 3.85-3.77 (m, 3H) 3.16-2.17 (m, 6H) 2.10 (br s, 3H) 1.88-1.80 (m, 6H) 1.73-1.62 (m, 8H) 0.31-0.21 (m, 9H).

### Procedure 4: Synthesis of Compound A-9

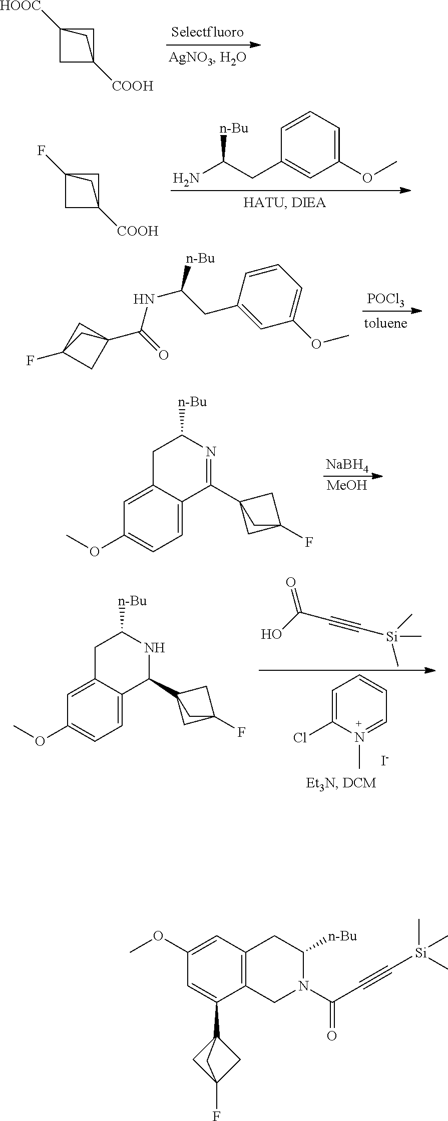

To a solution of bicyclo[1.1.1]pentane-1,3-dicarboxylic acid (2 g, 12.81 mmol, 1 eq) in H₂O (40 mL) was added 1-(chloromethyl)-4-fluoro-1,4-diazoniabicyclo[2.2.2]octane ditetrafluoroborate (8.17 g, 23.06 mmol, 1.8 eq) and AgNO₃ (435.19 mg, 2.56 mmol, 0.2 eq) under N₂ at 20° C. The resulting mixture was stirred at 55° C. for 12 hr to give a yellow solution. TLC (eluting with EtOAc) showed the reaction was completed. The reaction mixture was filtered, and the filtrate was extracted with MTBE (50 mL*3). The organic layers were dried over Na₂SO₄ and concentrated under vacuum to give 3-fluorobicyclo[1.1.1]pentane-1-carboxylic acid.

To a solution of 3-fluorobicyclo[1.1.1]pentane-1-carboxylic acid (550 mg, 4.23 mmol, 1 eq) in DMF (10 mL) was added (2S)-1-(3-methoxyphenyl)hexan-2-amine (963.93 mg, 4.65 mmol, 1.1 eq), DIEA (546.29 mg, 4.23 mmol, 736.25 μL, 1 eq) and HATU (1.61 g, 4.23 mmol, 1 eq). The mixture was stirred at 20° C. for 12 hr to give a yellow solution. TLC (eluting with PE/EtOAc=3/1) showed the reaction was completed. To the mixture was added H₂O (15 mL), then the reaction mixture was extracted with DCM (15 mL*3). The combined organic layers were dried over Na₂SO₄, filtered and concentrated. The residue was purified by column chromatography (SiO₂, Petroleum ether/Ethyl acetate=100/0 to 80/20) to give (S)-3-fluoro-N-(1-(3-methoxyphenyl)hexan-2-yl)bicyclo[1.1.1]pentane-1-carboxamide. ¹H NMR (400 MHz, CDCl₃) δ ppm 7.21 (t, J=8.0 Hz, 1H) 6.81-6.67 (m, 3H) 5.21 (br d, J=8.8 Hz, 1H) 4.21-4.06 (m, 1H) 3.91-3.71 (m, 3H) 2.89-2.66 (m, 2H) 2.25 (d, J=2.4 Hz, 6H) 1.63-1.20 (m, 6H) 1.01-0.72 (m, 3H).

To a solution of (S)-3-fluoro-N-(1-(3-methoxyphenyl)hexan-2-yl)bicyclo[1.1.1]pentane-1-carboxamide (980 mg, 3.07 mmol, 1 eq) in toluene (10 mL) was added POCl₃ (3.76 g, 24.55 mmol, 2.28 mL, 8 eq). The reaction was stirred at 140° C. for 1 hr to give a yellow solution. TLC (eluting with PE/EtOAc=3/1) showed the reaction was completed. The reaction mixture was poured into H₂O (15 mL), basified with sat. NaHCO₃ to pH=8, and extracted with EtOAc (15 mL*3). The organic layers were dried over Na₂SO₄, filtered and concentrated to give the crude product. The crude residue was purified by column chromatography (SiO₂, Petroleum ether/Ethyl acetate=100/0 to 88/12) to give (S)-3-butyl-1-(3-fluorobicyclo[1.1.1]pentan-1-yl)-6-methoxy-3,4-dihydroisoquinoline. ¹H NMR (400 MHz, CDCl₃) δ ppm 7.50 (d, J=8.8 Hz, 1H) 6.82-6.66 (m, 2H) 3.90-3.78 (m, 3H) 3.55-3.38 (m, 1H) 2.74 (dd, J=15.6, 5.2 Hz, 1H) 2.49-2.42 (m, 7H) 1.79-1.30 (m, 6H) 0.98-0.82 (m, 3H).

A solution of (S)-3-butyl-1-(3-fluorobicyclo[1.1.1]pentan-1-yl)-6-methoxy-3,4-dihydroisoquinoline (980 mg, 3.25 mmol, 1 eq) in MeOH (10 mL) was prepared. Then a solution of NaBH₄ (123.01 mg, 3.25 mmol, 1 eq) in MeOH (5 mL) was added dropwise at 20° C. The reaction was stirred 0.5 hr to give a yellow solution. LCMS and TLC (eluting with PE/EtOAc=3/1) showed the reaction was completed. Then NaHCO₃ (40 mL) was added, and the reaction mixture was extracted with EtOAc (30 mL*3). The organic layers were diluted with brine and dried over Na₂SO₄, filtered and concentrated to give crude product. The crude product was purified by flash column (SiO₂, eluting with PE/EtOAc=0% to 15%) to give (1S,3S)-3-butyl-1-(3-fluorobicyclo[1.1.1]pentan-1-yl)-6-methoxy-1,2,3,4-tetrahydroisoquinoline. LC-MS (m/z): 304.3 [M+H]⁺. ¹H NMR (400 MHz, CDCl₃) δ ppm 6.89 (d, J=8.4 Hz, 1H) 6.74-6.58 (m, 2H) 4.25 (s, 1H) 3.87-3.72 (m, 3H) 3.09-3.06 (m, 1H) 2.73-2.68 (m, 1H) 2.50-2.38 (m, 1H) 2.08-1.95 (m, 6H) 1.56-1.33 (m, 6H) 0.95 (br t, J=6.8 Hz, 3H).

To a solution of 3-trimethylsilylprop-2-ynoic acid (93.75 mg, 659.17 μmol, 2 eq) in DCM (6 mL) was added 2-chloro-1-methyl-pyridin-1-ium iodide (126.30 mg, 494.38 μmol, 1.5 eq). The mixture was stirred at 20° C. for 0.5 hr to give a yellow suspension. Then a solution of (1S,3S)-3-butyl-1-(3-fluorobicyclo[1.1.1]pentan-1-yl)-6-methoxy-1,2,3,4-tetrahydroisoquinoline (100 mg, 329.58 μmol, 1 eq) and TEA (50.03 mg, 494.37 μmol, 68.81 μL, 1.50 eq) in DCM (5 mL) was added dropwise. The resulting mixture was stirred at 20° C. for 1 hr to give a yellow solution. LCMS showed the reaction was not completed. Stirring was continued at 20° C. for 1 hr. TLC (eluting with PE/EtOAc=3/1) showed the reaction was completed. To the mixture was added H₂O (15 mL), and the reaction mixture was extracted with DCM (15 mL*3). The combined organic layers were dried over Na₂SO₄, filtered and concentrated. The crude product obtained was purified by flash column (SiO₂, eluting with PE/EtOAc=0% to 12%) to give 1-((1S,3S)-3-butyl-1-(3-fluorobicyclo[1.1.1]pentan-1-yl)-6-methoxy-3,4-dihydroisoquinolin-2(1H)-yl)-3-(trimethylsilyl)prop-2-yn-1-one. LC-MS (m/z): 428.2 [M+H]⁺. ¹H NMR (400 MHz, CDCl₃) δ ppm 7.03-6.89 (m, 1H) 6.85-6.64 (m, 2H) 5.51-5.37 (m, 1H) 4.55-4.38 (m, 1H) 3.89-3.77 (m, 3H) 3.09-2.87 (m, 1H) 2.74 (br d, J=15.2 Hz, 1H) 2.01-1.71 (m, 6H) 1.28-0.98 (m, 6H) 0.89-0.79 (m, 3H) 0.35-0.17 (m, 9H).

### Procedure-5: Synthesis of Compound A-10

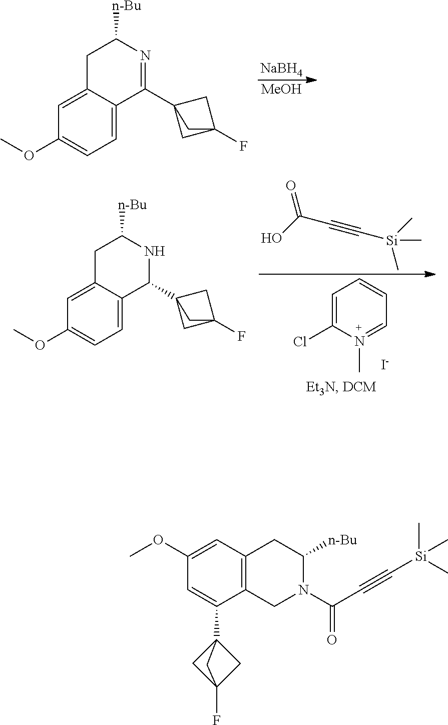

A solution of (S)-3-butyl-1-(3-fluorobicyclo[1.1.1]pentan-1-yl)-6-methoxy-3,4-dihydroisoquinoline (100 mg, 331.79 μmol, 1 eq) in MeOH (2 mL) at −78° C. under N₂ was prepared. Then a solution of NaBH₄ (12.55 mg, 331.79 μmol, 1 eq) in MeOH (2 mL) was added dropwise at −78° C. The reaction was stirred 0.5 hr to give a yellow solution. LCMS showed the reaction was completed. Then a sat. NaHCO₃ solution (40 mL) was added, and the reaction mixture was extracted with EtOAc (30 mL*3). The organic layers were diluted with a sat. NaCl solution and dried over Na₂SO₄, filtered and concentrated to give (1R,3S)-3-butyl-1-(3-fluorobicyclo[1.1.1]pentan-1-yl)-6-methoxy-1,2,3,4-tetrahydroisoquinoline. ¹H NMR (400 MHz, CDCl₃) δ ppm 7.07-6.85 (m, 1H) 6.79-6.68 (m, 1H) 6.62 (d, J=2.0 Hz, 1H) 4.35 (s, 1H) 3.79 (s, 3H) 2.86-2.60 (m, 2H) 2.52-2.39 (m, 1H) 2.04-1.97 (m, 4H) 1.66-1.30 (m, 9H) 1.01-0.88 (m, 3H).

To a solution of 3-trimethylsilylprop-2-ynoic acid (63.28 mg, 444.94 μmol, 1.5 eq) in DCM (2 mL) was added 2-chloro-1-methyl-pyridin-1-ium iodide (113.67 mg, 444.94 μmol, 1.5 eq). The mixture was stirred at 20° C. for 0.5 hr to give a yellow suspension. Then a solution of (1R,3S)-3-butyl-1-(3-fluorobicyclo[1.1.1]pentan-1-yl)-6-methoxy-1,2,3,4-tetrahydroisoquinoline (90.00 mg, 296.63 μmol, 1 eq) and TEA (45.02 mg, 444.94 μmol, 61.93 μL, 1.5 eq) in DCM (2 mL) was added dropwise. The resulting mixture was stirred at 20° C. for 1 hr to give a yellow solution. LCMS and TLC (PE/EtOAc=3:1) showed the reaction was completed. To the mixture was added H₂O (15 mL), and the reaction mixture was extracted with DCM (15 mL*3). The combined organic layers were dried over Na₂SO₄, filtered and concentrated. The resulting crude product was purified by flash column (SiO₂, eluting with PE/EtOAc=0% to 15%) to give 1-((1R,3S)-3-butyl-1-(3-fluorobicyclo[1.1.1]pentan-1-yl)-6-methoxy-3,4-dihydroisoquinolin-2(1H)-yl)-3-(trimethylsilyl)prop-2-yn-1-one. LC-MS (m/z): 428.2 [M+H]⁺. ¹H NMR (400 MHz, CDCl₃) δ ppm 7.10-6.90 (m, 1H) 6.83-6.67 (m, 2H) 5.90-5.73 (m, 1H) 4.57-4.39 (m, 1H) 3.88-3.77 (m, 3H) 3.17-2.90 (m, 1H) 2.79-2.71 (m, 1H) 1.97-1.71 (m, 6H) 1.38-1.08 (m, 6H) 0.87-0.75 (m, 3H) 0.34-0.18 (m, 9H).

### Procedure 6: Synthesis of Compound A-11

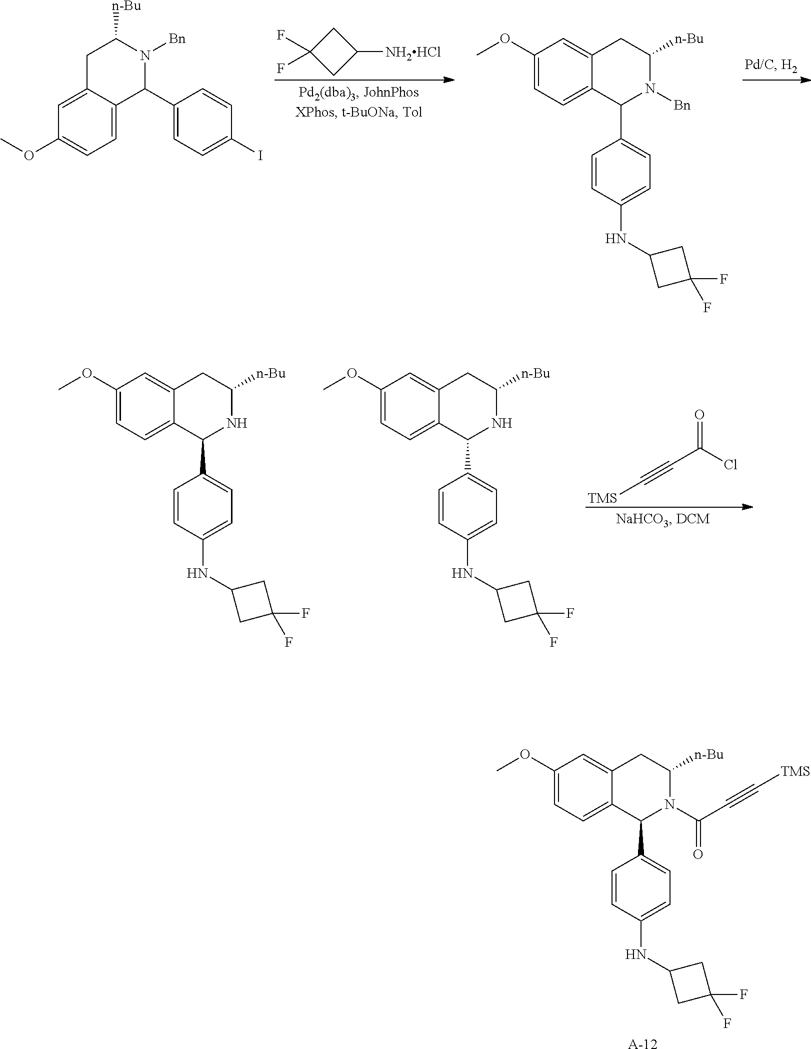

To a solution of (3S)-2-benzyl-3-butyl-1-(4-iodophenyl)-6-methoxy-1,2,3,4-tetrahydroisoquinoline (200 mg, 391.06 μmol, 1 eq) in toluene (5 mL), was added 3,3-difluorocyclobutanamine (168.42 mg, 1.17 mmol, 3 eq, HCl salt), t-BuONa (187.91 mg, 1.96 mmol, 5 eq), XPhos (37.28 mg, 78.21 μmol, 0.2 eq), JohnPhos (23.34 mg, 78.21 μmol, 0.2 eq), and Pd₂(dba)₃ (35.81 mg, 39.11 μmol, 0.1 eq). The reaction mixture was stirred at 130° C. under N₂ for 12 hr to give a black suspension. LCMS showed the reaction was completed. The reaction mixture was filtered on celite and washed with EtOAc (30 mL). The filtrate was concentrated to give crude product. The crude product was purified by flash column (eluting with PE/EtOAc=0% to 30%) to give 4-((3S)-2-benzyl-3-butyl-6-methoxy-1,2,3,4-tetrahydroisoquinolin-1-yl)-N-(3,3-difluorocyclobutyl)aniline. ¹H NMR (400 MHz, CDCl₃) δ=7.42-7.28 (m, 4H), 7.24-7.00 (m, 4H), 6.72-6.60 (m, 3H), 6.50-6.44 (m, 2H), 4.65-4.56 (m, 1H), 3.85-3.70 (m, 5H), 3.66-3.40 (m, 2H), 3.08-3.00 (m, 2H), 2.85-2.40 (m, 4H), 1.30-1.10 (m, 6H), 0.90-0.82 (m, 3H).

To a solution of 4-((3S)-2-benzyl-3-butyl-6-methoxy-1,2,3,4-tetrahydroisoquinolin-1-yl)-N-(3,3-difluorocyclobutyl)aniline (90 mg, 183.44 μmol, 1 eq) in EtOH (10 mL) was added Pd/C (50 mg, 10% purity) and HCl (23.89 mg, 183.44 μmol, 23.42 μL, 28% purity, 1 eq) under N₂. The resulting suspension was degassed under vacuum and purged with H₂ several times. The mixture was stirred under H₂ (41.09 ug, 20.38 μmol) (15 psi) at 20° C. for 3 hr to give a black suspension. LCMS showed the reaction was completed. The reaction mixture was filtered on celite and washed with MeOH (30 mL). The filtrate was concentrated to give 4-((1S,3S)-3-butyl-6-methoxy-1,2,3,4-tetrahydroisoquinolin-1-yl)-N-(3,3-difluorocyclobutyl)aniline (trans-isomer) and 4-((1R,3S)-3-butyl-6-methoxy-1,2,3,4-tetrahydroisoquinolin-1-yl)-N-(3,3-difluorocyclobutyl)aniline (cis-isomer). The product was used in the next step without further purification.

To a solution of 3-trimethylsilylprop-2-ynoic acid (12.78 mg, 89.89 μmol, 0.9 eq) in DCM (5 mL), was added Et₃N (10.11 mg, 99.87 μmol, 13.90 μL, 1 eq). The reaction mixture was stirred at 20° C. for 0.5 hr. Then 4-((1S,3S)-3-butyl-6-methoxy-1,2,3,4-tetrahydroisoquinolin-1-yl)-N-(3,3-difluorocyclobutyl)aniline (40.00 mg, 99.87 μmol, 1 eq) and 2-chloro-1-methylpyridin-1-ium iodide (22.96 mg, 89.89 μmol, 0.9 eq) was added dropwise at 0° C. The reaction was stirred at 0° C. for 0.5 hr to give a solution. TLC (eluting with PE/EtOAc=2/1) showed the reaction was completed. The reaction mixture was quenched with H₂O (20 mL) and extracted with DCM (30 mL*2). The organic layers were washed with HCl (1N, 10 mL). The organic layers were then separated and washed with sat. NaHCO₃ (15 mL). The organic layer was again separated, and then dried over Na₂SO₄, filtered and concentrated to give crude product. The crude product was purified by flash column (eluting with PE/EtOAc=0% to 40%) to give 1-((1S,3S)-3-butyl-1-(4-((3,3-difluorocyclobutyl)amino)phenyl)-6-methoxy-3,4-dihydroisoquinolin-2(1H)-yl)-3-(trimethylsilyl)prop-2-yn-1-one. LC-MS (m/z): 525.3[M+H]⁺. ¹H NMR (400 MHz, DMSO-d6) δ=7.38-7.26 (m, 1H), 6.92-6.82 (m, 2H), 6.75-6.70 (m, 2H), 6.40-6.30 (m, 2H), 6.10-5.80 (m, 2H), 4.56-4.40 (m, 1H), 3.66 (s, 3H), 3.00-2.70 (m, 5H), 2.34-2.26 (m, 2H), 1.46-1.10 (m, 6H), 0.80-0.70 (m, 3H), 0.20-0.02 (m, 9H).

### Procedure 7: Synthesis of Compound A-12

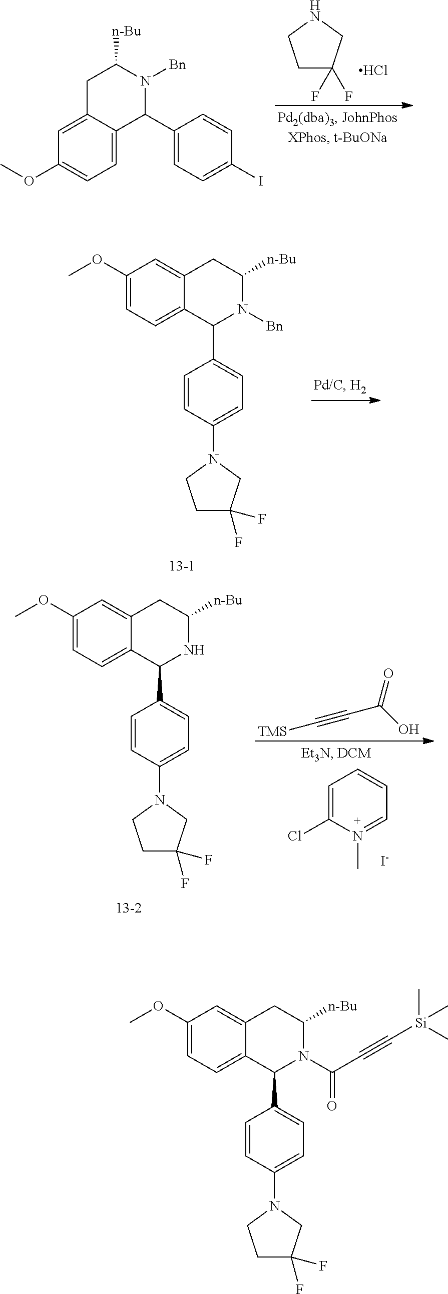

To a solution of (3S)-2-benzyl-3-butyl-1-(4-iodophenyl)-6-methoxy-1,2,3,4-tetrahydroisoquinoline (100 mg, 195.53 μmol, 1 eq) in toluene (5 mL) was added 3,3-difluoropyrrolidine hydrochloride (105.60 mg, 586.58 μmol, 3 eq, HCl), t-BuONa (93.95 mg, 977.64 μmol, 5 eq), XPhos (18.64 mg, 39.11 μmol, 0.2 eq), JohnPhos (11.67 mg, 39.11 μmol, 0.2 eq) and Pd₂(dba)₃ (17.90 mg, 19.55 μmol, 0.1 eq). The reaction was stirred at 125° C. under N₂ for 12 hr to give a black suspension. TLC and LCMS show the reaction was completed. To the reaction mixture was added DCM (50 ml), and the resulting mixture was concentrated to give crude product. The crude product was purified by flash column (SiO₂, eluting with PE/EtOAc=0-10%) to give intermediate 13-1. ¹H NMR (400 MHz, CDCl₃) δ ppm 7.26-7.19 (m, 1H) 6.98-6.90 (m, 2H) 6.85-6.75 (m, 1H) 6.70-6.66 (m, 1H) 6.46-6.35 (m, 2H) 6.24-6.19 (m, 1H) 4.61-4.48 (m, 1H) 3.82 (br s, 1H) 3.81-3.80 (m, 3H) 3.78-3.69 (m, 1H) 3.16-2.85 (m, 1H) 2.74-2.67 (m, 1H) 2.42-2.31 (m, 2H) 1.84-1.70 (m, 4H) 1.32-1.16 (m, 6H) 0.88-0.80 (m, 3H) 0.29-0.07 (m, 9H).

To a solution of intermediate 13-1 (180 mg, 366.88 μmol, 1 eq) in EtOH (10 mL) was added Pd/C (100 mg, 50% purity, 1.00 eq) and conc. HCl (110.00 mg, 1.32 mmol, 0.1 mL, 3.59 eq) under N₂. The suspension was degassed under vacuum and purged with H₂ several times. The mixture was stirred under H₂ (15 psi) at 25° C. for 12 hr to give a black suspension. LCMS showed the reaction was completed. The reaction mixture was filtered on celite and washed with MeOH (30 mL). The filtrate was concentrated to give the crude product. The crude product was purified by flash column (eluting with PE/EtOAc=0% to 40%) to give intermediate 13-2. ¹H NMR (400 MHz, CDCl₃) δ ppm 7.02-6.94 (m, 2H), 6.77-6.74 (m, 1H), 6.62-6.53 (m, 2H), 6.43-6.40 (m, 2H), 5.09 (s, 1H) 3.73 (s, 3H), 3.63-3.55 (m, 2H), 3.45-3.42 (m, 2H), 2.90-2.78 (m, 2H), 2.44-2.36 (m, 3H), 1.44-1.20 (m, 1H), 0.81-0.79 (t, J=6.8 Hz, 3H).

To a solution of 3-trimethylsilylprop-2-ynoic acid (31.96 mg, 224.72 μmol, 1 eq) in DCM (5 mL) was added 2-chloro-1-methylpyridin-1-ium iodide (57.41 mg, 224.72 μmol, 1 eq). The reaction was stirred at 20° C. for 0.5 hr. Then intermediate 13-2 and Et₃N (22.74 mg, 224.72 μmol, 31.28 μL, 1 eq) was added dropwise at 0° C. The reaction was stirred at 0° C. for 0.5 hr. TLC (eluting with PE/EtOAc=2/1) showed the reaction was completed. The reaction mixture was quenched with H₂O (20 mL) and extracted with DCM (30 mL*2). The organic layers were washed with HCl (1N, 10 mL). The organic layer was separated and washed sat. NaHCO₃ (15 mL). The organic layer was then separated, dried over Na₂SO₄ and concentrated to give the crude product. The crude product was purified by flash column (SiO₂, eluting with PE/EtOAc=0% to 40%) to give 1-((1S,3S)-3-butyl-1-(4-(3,3-difluoropyrrolidin-1-yl)phenyl)-6-methoxy-3,4-dihydroisoquinolin-2(1H)-yl)-3-(trimethylsilyl)prop-2-yn-1-one. LC-MS (m/z): 525.2 [M+H]⁺. ¹H NMR (400 MHz, CDCl₃) δ ppm 7.26-7.17 (m, 1H) 7.11-6.94 (m, 2H) 6.86-6.75 (m, 1H) 6.73-6.62 (m, 1H) 6.50-6.37 (m, 2H) 6.33-6.15 (m, 1H) 4.68-4.40 (m, 1H) 3.86-3.75 (m, 3H) 3.68-3.54 (m, 2H) 3.52-3.38 (m, 2H) 3.20-2.80 (m, 1H) 2.78-2.63 (m, 1H) 2.55-2.35 (m, 2H) 1.31-1.13 (m, 6H) 0.89-0.80 (m, 3H) 0.06-0.33 (m, 9H).

### Procedure 8: Synthesis of Compound A-15

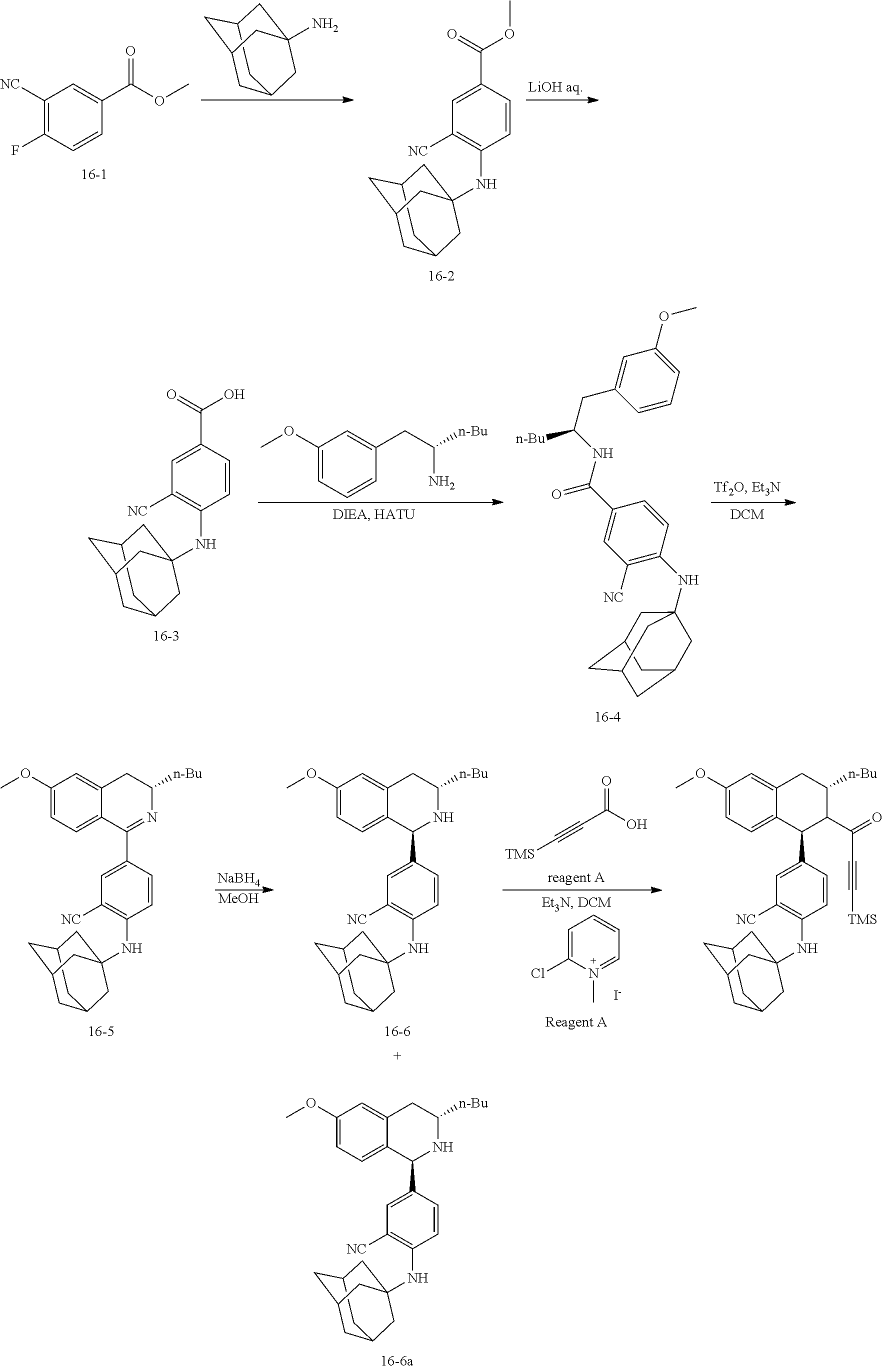

To a solution of 16-1 (9.56 g, 53.36 mmol, 1 eq) in DMSO (60 mL) were added adamantan-1-amine (12.11 g, 80.05 mmol, 1.5 eq) and Et₃N (16.20 g, 160.09 mmol, 22.28 mL, 3 eq). The reaction was stirred at 20° C. for 12 hr to give yellow solution. TLC showed the reaction was completed. The reaction was diluted with H₂O (30 mL) and extracted with MBTE (30 mL). The organic layers were dried over Na₂SO₄ and concentrated in vacuum to give 16-2 (11.8 g, crude) as a yellow solid. ¹H NMR (400 MHz, CDCl₃) δ=8.01 (d, J=2.4 Hz, 1H), 7.89 (dd, J=2.4 Hz, 8.8 Hz, 1H), 7.24 (d, J=8.8 Hz, 1H), 5.33 (s, 1H), 3.79 (s, 3H), 2.10-2.30 (m, 3H), 1.90-2.10 (m, 6H), 1.6-0-1.80 (m, 6H).

To a solution of 16-2 (11.8 g, 38.02 mmol, 1 eq) in THF (100 mL)/H2O (20 mL)/MeOH (20 mL) was added a solution of LiOH·H₂O (4.79 g, 114.05 mmol, 3 eq). The reaction was stirred at 20° C. for 12 hr to give yellow solution. TLC (eluting with:PE/EtOAc=3/1) showed the reaction was completed. The reaction was diluted with H₂O (30 mL) and extracted with MBTE (30 mL). The water layer was acidified to pH=5-6 and extracted with EtOAc (30 mL*3). The organic layers were dried over Na₂SO₄ and concentrated in vacuum to give 16-3 (8.3 g, crude) as a yellow solid. ¹H NMR (400 MHz, DMSO-d₆) δ 12.8 (brs, 1H), 7.98 (d, J=2 Hz, 1H), 7.88 (dd, J=2.0 Hz, 9.2 Hz, 1H), 7.22 (d, J=9.2 Hz), 5.27 (s, 1H), 1.50-1.80 (m, 6H), 1.90-2.20 (m, 9H).

To a solution of intermediate 16-3 (500 mg, 1.69 mmol, 1 eq) in DMF (10 mL) was added DIEA (436.10 mg, 3.37 mmol, 587.73 μL, 2 eq), (2S)-1-(3-methoxyphenyl)hexan-2-amine (349.76 mg, 1.69 mmol, 1 eq), and HATU (673.57 mg, 1.77 mmol, 1.05 eq) at 0° C. The reaction was allowed to stir at 25° C. for 12 hr to give a yellow solution. LCMS and TLC (eluting with PE/EtOAc=3/1) showed the reaction was completed. The reaction mixture was quenched with H₂O (30 mL) and extracted with MTBE (30 mL*3). The organic layers were dried over Na₂SO₄ and concentrated to give the crude product. The crude product was purified by flash column (eluting with PE/EtOAc=0% to 30%) to give intermediate 16-4. ¹H NMR (400 MHz, CDCl₃) δ=7.63-7.58 (m, 1H), 7.17-7.15 (t, J=8.0 Hz, 1H), 6.95-6.90 (m, 1H), 6.73-6.64 (m, 3H), 5.58-5.54 (m, 1H), 4.68 (s, 1H), 4.33-4.25 (m, 1H), 3.71 (s, 3H), 2.85-2.70 (m, 2H), 2.11 (s, 3H), 1.94 (s, 6H), 1.68-1.60 (m, 6H), 1.45-1.20 (m, 6H), 0.82-0.80 (t, J=6.8 Hz, 3H).

To a solution of intermediate 16-4 (500 mg, 1.03 mmol, 1 eq) in DCM (10 mL) was added 2-chloropyridine (350.69 mg, 3.09 mmol, 292.24 μL, 3 eq) and Tf₂O (871.41 mg, 3.09 mmol, 509.60 μL, 3 eq) at −78° C. The reaction was allowed to stir at 25° C. for 1 hr to give a yellow solution. LCMS showed the reaction was completed. The reaction mixture was quenched with sat. NaHCO₃ (20 mL) and extracted with DCM (30 mL*3). The organic layers were dried over Na₂SO₄ and concentrated to give the crude product. The crude product was purified by flash column (eluting with PE/EtOAc=0% to 30%) to give intermediate 16-5. ¹H NMR (400 MHz, CDCl₃) δ=7.60-7.52 (m, 1H), 7.19-7.14 (m, 1H), 7.00-6.92 (m, 1H), 6.73-6.64 (m, 3H), 4.52 (brs, 1H), 3.79 (s, 3H), 3.33 (brs, 1H), 2.72-2.60 (m, 1H), 2.49-2.40 (m, 1H), 2.10 (s, 3H), 1.96 (s, 6H), 1.68-1.60 (m, 6H), 1.45-1.20 (m, 6H), 0.91-0.86 (m, 3H).

To a solution of intermediate 16-5 (320 mg, 684.28 μmol, 1 eq) in MeOH (10 mL) was added NaBH₄ (129.44 mg, 3.42 mmol, 5 eq). The reaction was stirred at 25° C. for 0.5 hr to give a yellow solution. TLC (eluting with PE/EtOAc=2/1) showed the reaction was completed. The reaction mixture was quenched with sat. NH₄Cl (10 mL) and extracted with EtOAc (20 mL*3). The organic layers were dried over Na₂SO₄ and concentrated to give the crude product. The crude product was purified by flash column (eluting with PE/EtOAc=1/1) to give intermediate 16-6 and. ¹H NMR (400 MHz, CDCl₃) δ=7.19-7.10 (m, 1H), 7.00-6.87 (m, 1H), 6.84-6.80 (m, 1H), 6.74-6.68 (m, 1H), 6.64-6.60 (m, 1H), 5.00 (brs, 1H), 4.29 (s, 1H), 3.33 (brs, 1H), 3.76 (s, 3H), 2.84-2.70 (m, 2H), 2.52-2.45 (m, 1H), 2.08 (s, 3H), 1.90 (s, 6H), 1.68-1.60 (m, 6H), 1.45-1.20 (m, 6H), 0.82-0.80 (t, J=7.2 Hz, 3H).

To a solution of 3-trimethylsilylprop-2-ynoic acid (21.80 mg, 153.30 μmol, 0.9 eq) in DCM (5 mL) was added 2-chloro-1-methylpyridinium iodide (21.86 mg, 153.30 μmol, 0.9 eq). The reaction was stirred at 25° C. for 0.5 hr. Then the mixture was added dropwise to a solution of intermediate 16-6 (80 mg, 170.34 μmol, 1 eq) and Et₃N (17.24 mg, 170.34 μmol, 23.71 μL, 1 eq) in DCM (5 mL) at 0° C. The reaction stirred at 0° C. for 0.5 hr to give a yellow solution. TLC (eluting with PE/EtOAc=2/1) showed the reaction was completed. The reaction mixture was diluted with HCl (1N, 15 mL) and extracted with DCM (20 mL*3). The organic layers were dried over Na₂SO₄ and concentrated to give the crude product. The crude product was purified by flash column (eluting with PE/EtOAc=0% to 30%) to give 2-(((3R,5R,7R)-adamantan-1-yl)amino)-5-((1S,3S)-3-butyl-6-methoxy-2-(3-(trimethylsilyl)propioloyl)-1,2,3,4-tetrahydroisoquinolin-1-yl)benzonitrile. LC-MS (m/z): 594.2[M+H]⁺. ¹H NMR (400 MHz, DMSO-d6) δ=7.08-7.05 (m, 2H), 6.92-6.82 (m, 1H), 6.80-6.69 (m, 2H), 6.62-6.58 (m, 1H), 6.05-6.00 (m, 2H), 4.48-4.40 (m, 1H), 4.22-4.18 (m, 1H), 3.71 (s, 3H), 3.00-2.60 (m, 2H), 2.00 (s, 3H), 1.86-1.76 (m, 6H), 1.68-1.60 (m, 6H), 1.45-1.05 (m, 6H), 0.78-0.72 (m, 3H), 0.18-0.02 (m, 9H).

### Procedure 9: Synthesis of Compound A-16

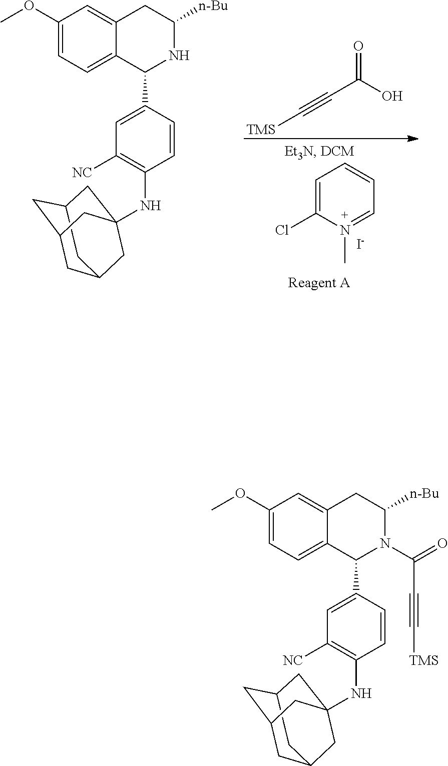

To a solution of 3-trimethylsilylprop-2-ynoic acid (32.10 mg, 225.70 μmol, 1 eq) in DCM (3 mL) was added 2-chloro-1-methyl-pyridin-1-ium iodide (57.66 mg, 225.70 μmol, 1 eq). The reaction was stirred at 25° C. for 0.5 hr. Then the mixture was added dropwise the a solution of 2-(((3S,5S,7S)-adamantan-1-yl)amino)-5-((1R,3S)-3-butyl-6-methoxy-1,2,3,4-tetrahydroisoquinolin-1-yl)benzonitrile and Et₃N (22.84 mg, 225.70 μmol, 31.41 μL, 1 eq) in DCM (10 mL) at 0° C. The reaction was stirred at 0° C. for 1 hr to give a yellow solution. LCMS and TLC (PE/EtOAc=3/1) showed the reaction was completed. The reaction mixture was quenched with H₂O (10 mL) and extracted with DCM (5 mL*3). The organic layers were washed with HCl (0.5N, 5 mL), then washed with sat. NaHCO₃ (10 mL). The organic layers were dried over Na₂SO₄ and concentrated to give the crude product. The crude product was purified by column chromatography (SiO₂, Petroleum ether/Ethyl acetate=100/0 to 90/10) to give 2-(((3S,5S,7S)-adamantan-1-yl)amino)-5-((1R,3S)-3-butyl-6-methoxy-2-(3-(trimethylsilyl)propioloyl)-1,2,3,4-tetrahydroisoquinolin-1-yl)benzonitrile. LC-MS (m/z): 594.5 [M+H]⁺. ¹H NMR (400 MHz, CHLOROFORM-d) δ ppm 7.38 (dd, J=9.07, 2.06 Hz, 1H) 6.90-7.16 (m, 3H) 6.71-6.86 (m, 2H) 6.51-6.66 (m, 1H) 4.58-4.69 (m, 1H) 4.32-4.48 (m, 1H) 3.78-3.88 (m, 3H) 2.93-3.19 (m, 1H) 2.38-2.72 (m, 1H) 2.15 (br s, 3H) 1.98 (br s, 6H) 1.67-1.75 (m, 6H) 1.26 (s, 2H) 1.01-1.21 (m, 4H) 0.71-0.82 (m, 3H) 0.18-0.30 (m, 9H).

### Procedure 10: Synthesis of Compound A-25

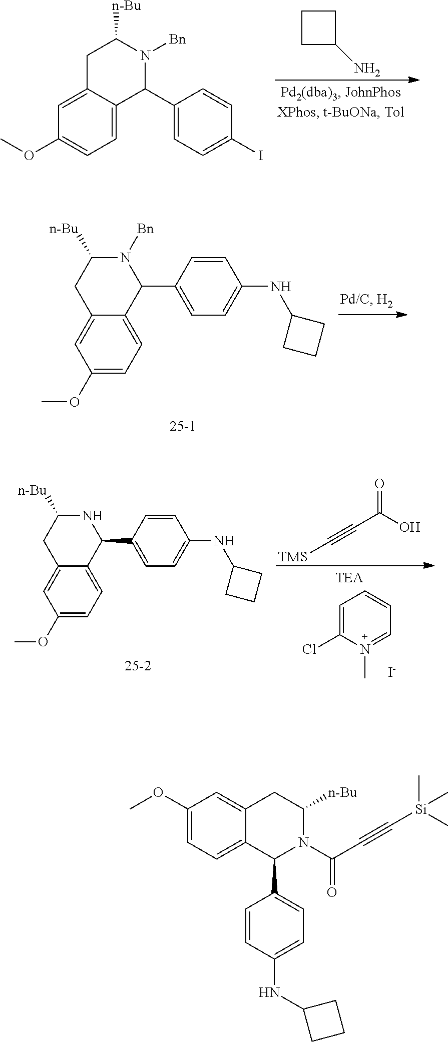

To a solution of (3S)-2-benzyl-3-butyl-1-(4-iodophenyl)-6-methoxy-1,2,3,4-tetrahydroisoquinoline (300 mg, 586.58 μmol, 1 eq) in toluene (5 mL) was added cyclobutanamine (125.15 mg, 1.76 mmol, 150.79 μL, 3.00 eq), t-BuONa (281.86 mg, 2.93 mmol, 5 eq), XPhos (55.93 mg, 117.32 μmol, 0.2 eq), JohnPhos (35.01 mg, 117.32 μmol, 0.2 eq), and Pd₂(dba)₃ (53.71 mg, 58.66 μmol, 0.1 eq). The reaction was stirred at 125° C. under N₂ for 12 hr to give a black suspension. LCMS and TLC (PE:EtOAc=10:1) showed the reaction was completed. To the reaction mixture was added DCM (50 ml), and the mixture was concentrated to give crude product. The crude product was purified by flash column (SiO₂, eluting with PE/EtOAc=0-10%) to give intermediate 25-1. ¹H NMR (400 MHz, CDCl₃) δ ppm 7.42-7.29 (m, 4H) 7.25-7.18 (m, 1H) 7.13-6.93 (m, 2H) 6.84-6.62 (m, 3H) 6.50-6.39 (m, 2H) 5.34-5.28 (m, 2H) 4.63-4.52 (m, 1H) 3.90-3.83 (m, 1H) 3.82-3.78 (m, 3H) 3.78-3.70 (m, 1H) 3.65-3.40 (m, 2H) 3.09-2.99 (m, 1H) 2.83-2.73 (m, 1H) 2.72-2.57 (m, 1H) 2.49-2.30 (m, 2H) 1.87-1.74 (m, 4H) 1.34-1.15 (m, 6H) 0.87-0.76 (m, 3H).

To a solution of intermediate 25-1 (380 mg, 835.82 μmol, 1 eq) in EtOH (20 mL) was added Pd/C (100 mg, 835.82 μmol, 50% purity, 1 eq) and HCl (12 M, 69.65 μL, 1 eq) under N₂. The suspension was degassed under vacuum and purged with H₂ several times. The mixture was stirred under H₂ (1.68 mg, 835.82 μmol, 1 eq) at 25° C. for 12 hr to give a black suspension. LCMS and TLC (eluting with PE/EtOAc=1/1) showed the reaction was completed. The reaction mixture was filtered on celite and washed with MeOH (30 mL). The filtrate was concentrated to give the crude product. The crude product was purified by flash column (eluting with PE/EtOAc=0% to 40%) to give intermediate 25-2. ¹H NMR (400 MHz, CDCl₃) δ ppm 6.96-6.92 (m, 2H), 6.88-6.84 (m, 1H), 6.75-6.65 (m, 1H), 6.49-6.36 (m, 2H), 5.16 (s, 1H), 3.92-3.86 (m, 1H), 3.81 (s, 3H), 3.68-3.54 (m, 1H), 3.03-2.91 (m, 1H), 2.88-2.86 (m, 1H), 2.65-2.60 (m, 1H), 2.42-2.39 (m, 1H), 1.83-1.73 (m, 2H), 1.47-1.31 (m, 6H), 0.88-0.84 (m, 3H).

To a solution of 3-trimethylsilylprop-2-ynoic acid (21.07 mg, 148.14 μmol, 0.9 eq) in DCM (5 mL) was added 2-chloro-1-methylpyridin-1-ium iodide (37.85 mg, 148.14 μmol, 0.9 eq). The reaction was stirred at 20° C. for 0.5 hr. Then intermediate 25-2 (60 mg, 164.60 μmol, 1 eq) and Et₃N (16.66 mg, 164.60 μmol, 22.91 μL, 1 eq) was added dropwise at 0° C. The reaction was stirred at 0° C. for 0.5 hr to give a solution. TLC (PE:EtOAc=2:1) showed the reaction was completed. The reaction mixture was quenched with H₂O (20 mL) and extracted with DCM (30 mL*2). The organic layers were washed with HCl (1N, 10 mL) and separated. The organic layer was then washed sat. NaHCO₃ (15 mL) and separated. The organic layer was then dried over Na₂SO₄ and concentrated to give 1-((1S,3S)-3-butyl-1-(4-(cyclobutylamino)phenyl)-6-methoxy-3,4-dihydroisoquinolin-2(1H)-yl)-3-(trimethylsilyl)prop-2-yn-1-one. MS (m/z): 489.5 [M+H]⁺. ¹H NMR (400 MHz, CDCl₃) δ ppm 7.26-7.19 (m, 1H) 6.98-6.90 (m, 2H) 6.85-6.75 (m, 1H) 6.70-6.66 (m, 1H) 6.46-6.35 (m, 2H) 6.24-6.19 (m, 1H) 4.61-4.48 (m, 1H) 3.82 (br s, 1H) 3.81-3.80 (m, 3H) 3.78-3.69 (m, 1H) 3.16-2.85 (m, 1H) 2.74-2.67 (m, 1H) 2.42-2.31 (m, 2H) 1.84-1.70 (m, 4H) 1.32-1.16 (m, 6H) 0.88-0.80 (m, 3H) 0.29-0.07 (m, 9H).

### Procedure 11: Synthesis of Compound A-26

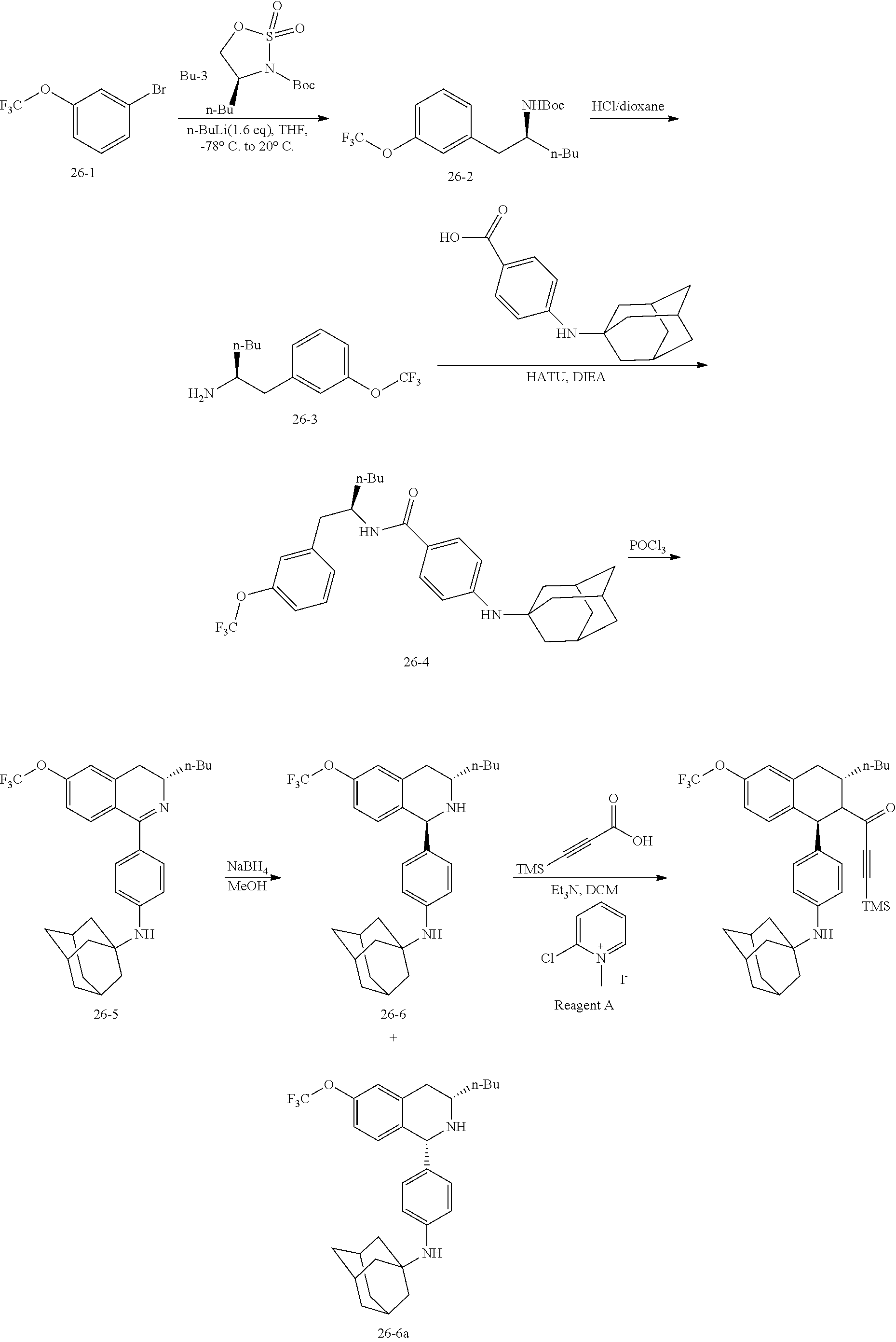

To a solution of starting material 26-1 (6.5 g, 26.97 mmol, 4.01 mL, 1.5 eq) in THF (50 mL) was added dropwise n-BuLi (2.5 M, 11.51 mL, 1.6 eq) at −78° C. The reaction was stirred at −78° C. for 0.5 hr. Then reagent Bu-3 (5.02 g, 17.98 mmol, 1 eq) in THF (30 mL) was added dropwise at −78° C. The reaction was allowed to stir at 20° C. for 12 hr to give a yellow solution. LCMS and TLC (eluting with PE/EtOAc=6/1) showed the reaction was completed. The reaction mixture was quenched with sat. citric acid (20 mL) and extracted with EtOAc (30 mL*3). The organic layers were dried over Na₂SO₄ and concentrated to give the crude product. The crude product was purified by flash column (eluting with PE/EtOAc=0% to 15%) to give intermediate 26-2. ¹H NMR (400 MHz, CDCl₃) δ ppm 7.26-7.20 (m, 1H), 7.18-7.08 (m, 1H), 7.02 (br dd, J=19.95, 7.57 Hz, 1H), 4.31-4.14 (m, 1H), 2.78-2.58 (m, 1H), 1.38-1.30 (m, 8H), 1.29 (br s, 2H), 1.27-1.23 (m, 1H), 1.21 (br dd, J=5.44, 2.06 Hz, 1H), 0.83-0.77 (m, 3H).

Intermediate 26-2 (1.6 g, 4.43 mmol, 1 eq) was dissolved in HCl/dioxane (4 M, 1.11 mL, 1 eq) at 0° C. The reaction was allowed to stir at 25° C. for 12 hr to give a colorless solution. LCMS showed the reaction was completed. The reaction mixture was concentrated directly to give intermediate 26-3. The product was used in the next step without further purification. ¹H NMR (400 MHz, MeOD) δ ppm 7.53-7.47 (m, 1H), 7.47 (br s, 1H), 7.36-7.29 (m, 1H), 7.25 (br s, 2H), 3.50 (quin, J=6.69 Hz, 1H), 3.01 (d, J=7.13 Hz, 2H), 1.70-1.58 (m, 2H), 1.42-1.32 (m, 4H), 0.96-0.91 (m, 1H), 0.98-0.90 (m, 3H).

To a solution of 4-(1-adamantylamino)benzoic acid (1.04 g, 3.83 mmol, 1 eq) in DMF (20 mL) was added DIEA (989.30 mg, 7.65 mmol, 1.33 mL, 2 eq), intermediate 26-3 (1 g, 3.83 mmol, 1 eq) and HATU (1.53 g, 4.02 mmol, 1.05 eq) at 0° C. The reaction was allowed to stir at 0-25° C. for 12 hr to give a black-brown liquid. LCMS and TLC (eluting with PE/EtOAc=3/1) showed the reaction was completed. The reaction mixture was quenched with H₂O (100 mL) and extracted with MTBE (30 mL*3). The organic layers were dried over Na₂SO₄ and concentrated to give the crude product. The crude product was purified by flash column (eluting with PE/EtOAc=0% to 20%) to give intermediate 26-4. ¹H NMR (400 MHz, CDCl₃) δ ppm 7.45-7.39 (m, 2H), 7.27-7.21 (m, 1H), 7.09 (d, J=7.63 Hz, 1H), 7.03-6.97 (m, 2H), 6.65-6.59 (m, 2H), 4.33-4.23 (m, 1H), 2.86-2.77 (m, 2H), 2.06 (br s, 3H), 1.91-1.84 (m, 7H), 1.63 (br s, 6H), 1.31-1.27 (m, 2H), 1.19 (t, J=7.13 Hz, 4H), 0.81-0.76 (m, 4H).

To solution of intermediate 26-4 (620 mg, 1.20 mmol, 1 eq) in DCM (10 mL) was added 2-chloropyridine (410.41 mg, 3.61 mmol, 342.00 μL, 3 eq) and Tf₂O (1.02 g, 3.61 mmol, 596.34 μL, 3 eq) at −78° C. The reaction was allowed to stir at 25° C. for 12 hr to give a yellow solution. LCMS and TLC (eluting with PE/EtOAc=3/1) showed the reaction was completed. The reaction mixture was quenched with sat. NaHCO₃ (20 mL) and extracted with DCM (30 mL*3). The organic layers were dried over Na₂SO₄ and concentrated to give the crude product. The crude product was purified by flash column (eluting with PE/EtOAc=0% to 30%) to give intermediate 26-5. ¹H NMR (400 MHz, CDCl₃) δ ppm 7.53-7.48 (m, 1H), 7.48-7.40 (m, 3H), 7.13-7.05 (m, 3H), 3.55-3.38 (m, 1H), 2.98-2.89 (m, 1H), 2.67-2.53 (m, 1H), 2.00-1.92 (m, 9H), 1.75-1.70 (m, 8H), 1.46-1.35 (m, 4H), 0.98-0.93 (m, 3H).

To a solution of intermediate 26-5 (170 mg, 342.32 μmol, 1 eq) in MeOH (5 mL) was added NaBH₄ (64.75 mg, 1.71 mmol, 5 eq). The reaction was stirred at 25° C. for 0.5 hr to give a yellow solution. TLC (eluting with PE/EtOAc=2/1) showed the reaction was completed. The reaction was quenched with sat. NH₄Cl (10 mL) and extracted with EtOAc (20 mL*3). The organic layers were dried over Na₂SO₄ and concentrated to give the crude product. The crude product was purified by flash column (eluting with PE/EtOAc=0% to 50%) to give intermediate 26-6 and intermediate 26-6a. ¹H NMR (400 MHz, CDCl₃) δ ppm 7.12-7.06 (m, 2H), 6.96-6.84 (m, 2H), 6.79-6.74 (m, 3H), 4.95 (s, 1H), 3.08-2.82 (m, 3H), 2.13 (s, 3H), 1.90 (s, 6H), 1.72-1.66 (m, 6H), 1.44-1.35 (m, 6H), 0.96-0.92 (m, 3H).

To a solution of 3-trimethylsilylprop-2-ynoic acid (7.96 mg, 55.95 μmol, 0.9 eq) in DCM (3 mL) was added 2-chloro-1-methyl-pyridin-1-ium iodide (14.30 mg, 55.95 μmol, 0.9 eq). The reaction was stirred at 25° C. for 0.5 hr. Then the mixture was added dropwise to a solution of intermediate 26-6 (31.00 mg, 62.17 μmol, 1 eq) and Et₃N (6.29 mg, 62.17 μmol, 8.65 μL, 1 eq) in DCM (3 mL) at 0° C. LCMS showed the reaction was completed. The reaction mixture was quenched with H₂O (0.5N, 10 mL) and extracted with DCM (20 mL*3). The organic layers were washed with sat. NaHCO₃ (15 mL). The organic layers were dried over Na₂SO₄ and concentrated to give the crude product. The crude product was purified by flash column (eluting with PE/EtOAc=0% to 30%) to give 1-((1S,3S)-1-(4-(((3R,5R,7R)-adamantan-1-yl)amino)phenyl)-3-butyl-6-(trifluoromethoxy)-3,4-dihydroisoquinolin-2(1H)-yl)-3-(trimethylsilyl)prop-2-yn-1-one. MS (m/z): 623.4 [M+H]⁺

### Procedure 12: Synthesis of Compound A-29

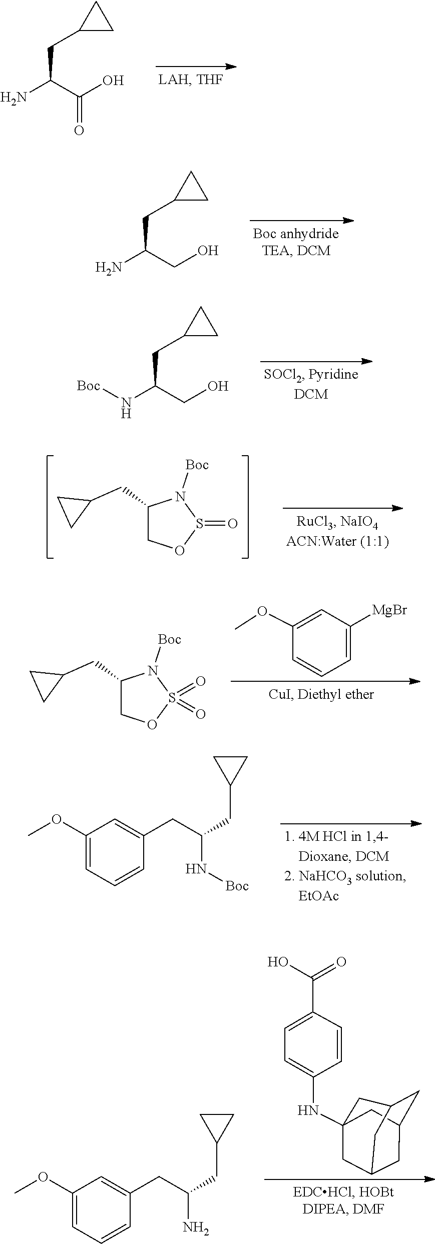

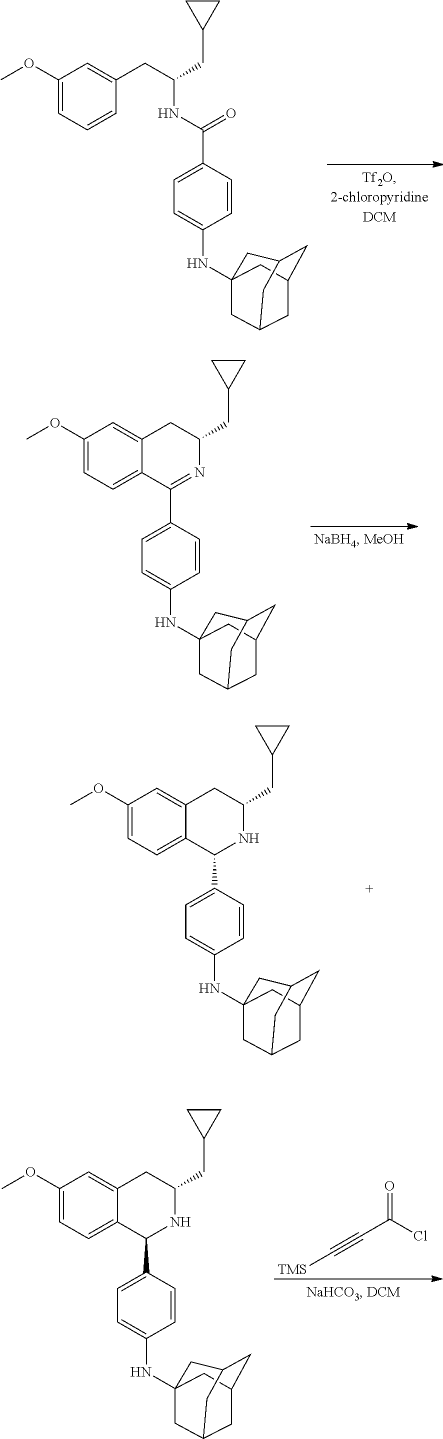

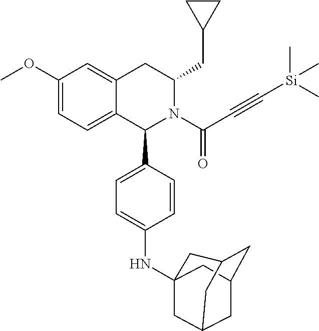

To a solution of (2S)-2-amino-3-cyclopropylpropanoic acid (5.0 g, 38.7 mmol, 1 eq) in THF (100.0 mL) at 0° C. was added a 1 M LAH solution in THF (77.4 mL, 38.7 mmol, 2 eq). The reaction mixture was warmed to room temperature, and then the mixture was stirred at 70° C. for 16 hr under nitrogen atmosphere. TLC (5% MeOH in DCM) showed the reaction was incomplete. The reaction mixture was cooled to room temperature. The reaction was diluted with diethyl ether (30 mL), cooled to 0° C., and quenched with dropwise addition of 2.94 mL of water and 2.94 mL of 15% aqueous sodium hydroxide solution, followed by the addition of 8.8 mL of water. The reaction mixture was stirred for 15 mins at room temperature. Then sodium sulphate was added to the reaction mixture, and the mixture was stirred for another 10 mins. The reaction mixture was filtered through a celite bed, washed with EtOAc (150 mL) and the filtrate was concentrated under reduced pressure to afford (2S)-2-amino-3-cyclopropylpropan-1-ol. LC-MS (ES) m/z=116.2 [M+H]⁺.

To a solution of (2S)-2-amino-3-cyclopropylpropan-1-ol (4.90 g, 42.5 mmol, 1.0 eq) in DCM (70 mL) under nitrogen atmosphere was added TEA (12 mL, 42.5 mmol, 2.0 eq) at 0° C. dropwise, and the resulting mixture was stirred for 5 mins, then di-tert-butyl dicarbonate (11.7 mL, 42.5 mmol, 1.2 eq) was added. The reaction was stirred at room temperature for 16 hr. The reaction was monitored by TLC (5% MeOH-DCM). After completion of the reaction, the reaction mixture was diluted with DCM (100 mL), washed with water (50 mL) and brine (25 mL), dried over Na₂SO₄, filtered and concentrated under reduced pressure to give the crude product. The crude was purified by silica gel column chromatography using 2-3% MeOH in DCM as an eluent to afford tert-butyl N-[(2S)-1-cyclopropyl-3-hydroxypropan-2-yl]carbamate. ¹H NMR (400 MHz, CDCl₃) δ ppm 0.08 (s, 2H), 0.48 (d, J=7.2 Hz, 2H), 0.67-0.69 (m, 1H), 1.44 (s, 9H), 1.62 (s, 2H), 2.47 (bs, 1H), 3.62-3.72 (m, 3H), 4.73 (bs, 1H).

To a solution of thionyl chloride (3.96 mL, 21.8 mmol, 2.5 eq) in DCM (50.0 mL) at −40° C. was added tert-butyl N-[(2S)-1-cyclopropyl-3-hydroxypropan-2-yl]carbamate (4.70 g, 21.8 mmol, 1.0 eq) in DCM (20 mL) and pyridine (9.14 mL, 21.8 mmol, 5.2 eq.). The mixture was stirred at −40° C. for 2 h under nitrogen atmosphere. TLC (20% EtOAc in hexane) showed the reaction was completed. The reaction was diluted with DCM:EtOAc (50 mL:50 mL) and the precipitate was filtered. The filtrate was washed with brine (50 mL). The organic layer was dried over anhydrous Na₂SO₄, filtered and concentrated under reduced pressure to get tert-butyl (4S)-4-(cyclopropylmethyl)-2-oxo-1,2λ⁴,3-oxathiazolidine-3-carboxylate.

Tert-butyl (4S)-4-(cyclopropylmethyl)-2-oxo-1,2λ⁴,3-oxathiazolidine-3-carboxylate (4.80 g, 18.4 mmol, 1.0 eq) was dissolved in MeCN (30 mL) and then ruthenium chloride (0.0533 g, 18.4 mmol, 0.014 eq) and sodium periodate (4.32 g, 18.4 mmol, 1.1 eq) were added at 0° C. Then water (30 mL) was added. The mixture was stirred at 0° C. for 15 mins and then at room temperature for 2 hr. TLC (20% EtOAc in hexane) showed the reaction was completed. The reaction mixture was filtered through a celite bed and the bed was washed with EtOAc (100 mL). The filtrate was washed with water (30 mL) and brine solution (20 mL). The organic layer was dried over anhydrous Na₂SO₄, filtered and concentrated under reduced pressure to get the crude product. The crude was purified by flash chromatography using 10-15% EtOAc in hexane as an eluent to give tert-butyl (4S)-4-(cyclopropylmethyl)-2,2-dioxo-1,2R⁶,3-oxathiazolidine-3-carboxylate. ¹H NMR (400 MHz, CDCl3) δ ppm 0.15-0.16 (m, 2H), 0.48-0.59 (m, 2H), 0.64-0.66 (m, 1H), 1.48 (s, 9H), 1.67-1.73 (m, 1H), 1.79-1.87 (m, 1H), 4.37 (s, 1H), 4.46 (d, J=8.8 Hz, 1H), 4.65-4.69 (m, 1H).

To a solution of copper(I) iodide (206 mg, 0.1 eq., 1.08 mmol) in diethyl ether (20 mL) was added bromo(3-methoxyphenyl)magnesium (21.6 mL, 2 eq., 21.6 mmol) dropwise over a period of 10 min at −20° C. (salt and ice mixture bath). The reaction mixture stirred for 30 min at −20° C. (salt and ice mixture bath). After this time, a solution of tert-butyl (4S)-4-(cyclopropylmethyl)-2,2-dioxo-1,2λ⁶,3-oxathiazolidine-3-carboxylate (3.00 g, 9.82 mmol) in diethyl ether (10 mL) was added at −20° C. (salt and ice mixture bath) dropwise over a period of 20 min to the reaction mass. The resulting mixture stirred for 3 hr at −20° C. The reaction was monitored by TLC (10% EtOAc in n-hexane). After the completion of the reaction, the reaction mixture quenched with 10% aqueous citric acid solution (100 mL) at −20° C. (salt and ice mixture bath). The mixture was allowed to warm to RT and stirred for 10 min. The mixture was filtered through a celite pad and washed with ethyl acetate thoroughly. The filtrate was washed with water (50 mL) and brine (30 mL), dried over anhydrous sodium sulfate, filtered and concentrated to give the crude product. The crude was purified by silica gel column chromatography using 4-5% EtOAc in n-hexane as an eluent to afford tert-butyl N-[(2S)-1-cyclopropyl-3-(3-methoxyphenyl)propan-2-yl]carbamate. LC-MS (m/z)=250.2 ([M+H]⁺ after cleavage of boc group. ¹H NMR (400 MHz, DMSO-d6) δ ppm −0.09 (s, 1H), 0.01 (s, 1H), 0.33-0.35 (m, 2H), 0.66 (s, 1H), 1.13-1.29 (s, 11H), 2.60-2.64 (m, 2H), 3.70 (s, 3H), 6.29-6.33 (m, 1H), 6.62 (d, J=8.8 Hz, 1H), 6.71 (s, 2H), 7.13 (t, J=7.2 Hz, 1H).

To a solution of tert-butyl N-[(2S)-1-cyclopropyl-3-(3-methoxyphenyl)propan-2-yl]carbamate (3.0 g, 9.82 mmol, 1.0 equiv.) in DCM (30.0 mL) under nitrogen atmosphere was added 4 M HCl in 1,4-dioxane (8.0 mL) dropwise, and the reaction mixture was stirred for 16 hr at room temperature. The reaction was monitored by TLC (50% EtOAc in n-hexane). After completion of the reaction, the reaction mixture was concentrated under reduced pressure to provide residue. The residue was then diluted with EtOAc (100 mL), and saturated sodium bicarbonate solution was added until pH ˜8. The mixture was stirred for 30 mins. The layers were separated and the aqueous layer was extracted with EtOAc (50 mL). Combined organic layers were dried over anhydrous sodium sulfate, filtered and concentrated under reduced pressure to afford (2S)-1-cyclopropyl-3-(3-methoxyphenyl)propan-2-amine. LC-MS (m/z)=206.2 ([M+H]⁺. ¹H NMR (400 MHz, DMSO-d6) δ ppm 0.08-0.07 (m, 2H), 0.34-0.40 (m, 2H), 0.76 (s, 1H), 1.09-1.22 (m, 2H), 1.43-1.45 (m, 2H), 2.37-2.47 (m, 1H), 2.61-2.66 (m, 1H), 2.91-2.93 (m, 1H), 3.70 (s, 3H), 6.72 (s, 3H), 7.16 (t, J=7.8 Hz, 1H).

To a solution of 4-[(adamantan-1-yl)amino]benzoic acid (2.51 g, 9.25 mmol) and (2S)-1-cyclopropyl-3-(3-methoxyphenyl)propan-2-amine (1.90 g, 9.25 mmol) in N,N-dimethylformamide (30.0 mL) was added N,N-dimethylpyridin-4-amine (2.83 g, 2.5 eq., 23.1 mmol), and the resulting mixture was stirred for 5 min. Then ({[3-(dimethylamino)propyl]imino}methylidene)(ethyl)amine hydrochloride (3.55 g, 2 eq., 18.5 mmol) was added at 0° C. This reaction mixture was stirred at room temperature for 16 hr. Progress of the reaction was monitored by TLC (30% ethyl acetate in n-hexane). After this time, the reaction mixture was diluted with EtOAc and saturated sodium bicarbonate solution. The organic layer was separated, washed with water and brine solution, dried over anhydrous sodium sulfate, filtered and concentrated under reduced pressure to get the crude product. The obtained crude product was purified by flash chromatography on silica gel. The desired product was eluted at 30% ethyl acetate in n-hexane. Fractions containing product were combined and concentrated under reduced pressure to get 4-[(adamantan-1-yl)amino]-N-[(2S)-1-(3-methoxyphenyl)hexan-2-yl]benzamide. LC-MS (ES) (m/z)=458.6 [M+H]⁺. ¹H NMR (400 MHz, DMSO) δ ppm 0.039-0.051 (m, 2H), 0.34 (d, J=7.6 Hz, 2H), 0.71 (bs, 1H), 1.29-1.47 (m, 2H), 1.64 (s, 6H), 1.81 (s, 6H), 2.05 (s, 3H), 2.77-2.79 (m, 2H), 3.67 (s, 3H), 4.16-4.19 (m, 1H), 5.52 (s, 1H), 6.69 (d, J=8.4 Hz, 3H), 6.76-6.78 (m, 2H), 7.13 (t, J=8.0 Hz, 1H), 7.51 (d, J=8.8 Hz, 2H), 7.70 (d, J=8.4 Hz, 1H).

To stirred solution of 4-[(adamantan-1-yl)amino]-N-[(2S)-1-cyclopropyl-3-(3-methoxyphenyl)propan-2-yl]benzamide (1.25 g, 2.73 mmol) and 2-chloropyridine (1.55 mL, 6 eq., 16.4 mmol) in DCM (30 mL) was added trifluoromethanesulfonic anhydride (15 mL, 89.2 mmol, 3.0 equiv) slowly via syringe dropwise at −78° C. After 5 min, the reaction mixture was placed in an ice-water bath and warmed to 0° C. After 5 min, the resulting solution was allowed to stir at room temperature for 1.5 hr. The reaction mixture was quenched with aqueous sodium hydroxide solution (50 mL, 1N) to neutralize the trifluoromethanesulfonate salts. Dichloromethane (50 mL) was added to dilute the mixture and the layers were separated. The aqueous layer was extracted with DCM (30 mL). The combined organic layer was washed with brine (25 mL), dried over anhydrous sodium sulfate and filtered. The organic layer was filtered and concentrated under reduced pressure to give crude (1S,3R,5S)—N-(4-((S)-3-(cyclopropylmethyl)-6-methoxy-3,4-dihydroisoquinolin-1-yl)phenyl)adamantan-1-amine. LCMS (ES) m/z: 440.6 [M+H]⁺. ¹H NMR (400 MHz, DMSO) δ ppm: 0.06-0.07 (m, 1H), 0.43 (d, J=7.2 Hz, 2H), 0.90 (bs, 1H), 1.32-1.49 (m, 2H), 1.40-1.49 (m, 1H) 1.64 (s, 6H), 1.80 (s, 6H), 2.06 (s, 3H), 2.55-2.58 (m, 1H), 2.86-2.89 (m, 1H), 3.45 (bs, 1H), 3.8 (s, 3H), 5.57 (ds, 1H), 6.77-6.85 (m, 3H), 6.93 (s, 1H), 7.28 (d, J=8.4, 3H).

To a solution of N-{4-[(3S)-3-(cyclopropylmethyl)-6-methoxy-3,4-dihydroisoquinolin-1-yl]phenyl}adamantan-1-amine (1.00 g, 2.27 mmol) in methanol (5.00 mL) was added sodium boranuide (258 mg, 3 eq., 6.81 mmol) at 0° C. portion wise. The suspension was stirred at RT for 2 hr. After this time, the reaction mixture was concentrated and the crude obtained was diluted with EtOAc and water. The organic layer was separated, washed with brine solution, dried over anhydrous sodium sulfate, filtered and concentrated under reduced pressure to give the crude product. The crude was purified by flash chromatography using 35-45% EtOAc in hexane as an eluent to give (1S,3R,5S)—N-(4-((1R,3S)-3-(cyclopropylmethyl)-6-methoxy-1,2,3,4-tetrahydroisoquinolin-1-yl)phenyl)adamantan-1-amine and (1S,3R,5S)—N-(4-((1S,3S)-3-(cyclopropylmethyl)-6-methoxy 1,2,3,4 tetrahydroisoquinolin-1-yl)phenyl)adamantan-1-amine. LC-MS (ES) (m/z)=442.6 [M+H]⁺. ¹H NMR (400 MHz, DMSO) δ ppm; 0.05 (bs, 2H), 0.34 (m, 2H), 0.66 (bs, 1H), 0.81-0.92 (m, 1H), 1.20 (s, 3H), 1.32-1.43 (m, 2H), 1.59 (s, 6H), 1.80 (s, 6H), 2.00 (s, 3H), 2.85-2.89 (m, 1H), 4.97 (s, 1H) 5.52 (s, 1H), 2.97 (bs, 1H), 6.62-6.72 (m, 4H), 6.78-6.87 (m, 3H).

To a solution of (3R,5R,7R)—N-(4-((1S,3S)-3-(cyclopropylmethyl)-6-methoxy-1,2,3,4-tetrahydroisoquinolin-1-yl)phenyl)adamantan-1-amine (50 mg, 112.96 μmol, 1 eq) in DCM (1 mL) was added NaHCO₃ (75.92 mg, 903.68 μmol, 35.15 μL, 8 eq) and 3-trimethylsilylprop-2-ynoyl chloride (0.2 M, 1.13 mL, 2 eq). The resulting mixture was stirred at 20° C. for 12 hr to give yellow suspension. LCMS and TLC (PE/EtOAc=3:1) showed the reaction were completed. The solution was washed H₂O (10 mL) and extracted with DCM (10 mL×3). The organic layer was dried over Na₂SO₄, filtrated and concentrated. The crude product provided was purified by flash column (SiO₂, eluting with PE/EtOAc=0% to 30%) to give 1-((1S,3S)-1-(4-(((3R,5R,7R)-adamantan-1-yl)amino)phenyl)-3-(cyclopropylmethyl)-6-methoxy-3,4-dihydroisoquinolin-2(1H)-yl)-3-(trimethylsilyl)prop-2-yn-1-one. LC-MS (m/z): 495.3 [M+H]⁺

¹H NMR (400 MHz, CDCl₃) δ ppm 7.29 (d, J=8.28 Hz, 1H) 7.21 (d, J=8.53 Hz, 1H) 6.91 (dd, J=15.18, 8.41 Hz, 2H) 6.80 (ddd, J=15.18, 8.28, 2.64 Hz, 1H) 6.74-6.70 (m, 1H) 6.64 (dd, J=11.04, 8.53 Hz, 2H) 6.26-6.15 (m, 1H) 4.82-4.56 (m, 1H) 3.81 (d, J=5.77 Hz, 3H) 3.22-3.10 (m, 1H) 3.03-2.81 (m, 2H) 2.13-2.00 (m, 3H) 1.83 (dd, J=11.42, 2.38 Hz, 6H) 1.59-1.41 (m, 6H) 1.35-0.79 (m, 3H) 0.72-0.55 (m, 1H) 0.53-0.32 (m, 2H) 0.16-0.05 (m, 2H)

### Procedure 13: Synthesis of Compound A-33

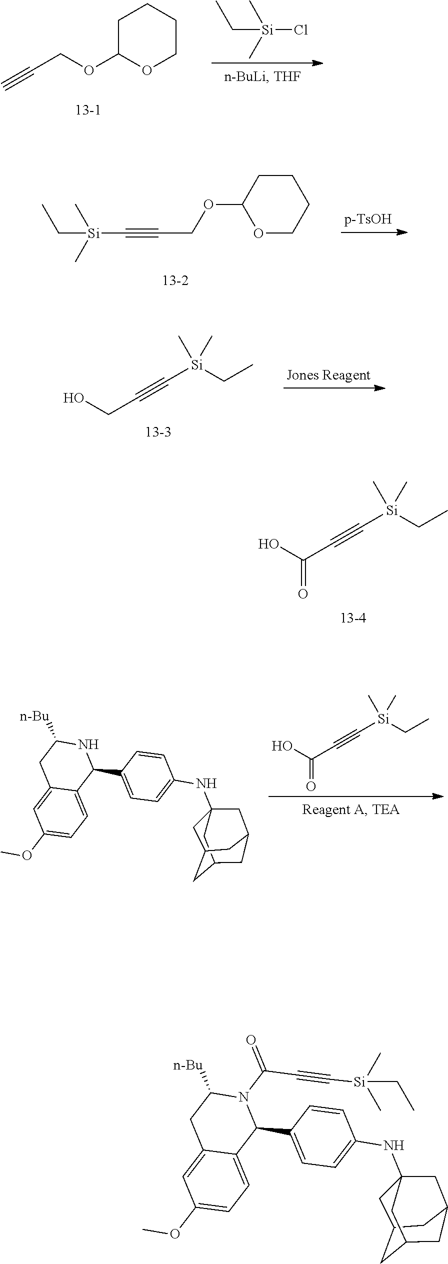

To a solution of 13-1 (1 g, 7.13 mmol, 1.00 mL, 1 eq) in THF (10 mL) was added dropwise n-BuLi (2.5 M, 3.42 mL, 1.2 eq) at −78° C. The reaction was stirred at −78° C. for 0.5 hr under N₂. Then chloro-ethyl-dimethyl-silane (1.05 g, 8.56 mmol, 1.2 eq) in THF (5 mL) was added dropwise at −78° C. The reaction was allowed to stir at 0° C. for 2 hr to give a yellow solution. TLC (eluting with:PE/EtOAc=10/1) showed the reaction was completed. The reaction mixture was quenched with Sat·NH₄Cl (20 mL) and extracted with EtOAc (30 mL×3). The organic layers were dried over Na₂SO₄ and concentrated to give 13-2. ¹H NMR (400 MHz, CDCl₃) δ ppm 4.77-4.74 (m, 1H), 3.74-3.65 (m, 1H), 3.44-3.35 (m, 1H), 1.70-1.38 (m, 8H), 0.85 (t, J=8.0 Hz, 3H), 0.50-0.42 (m, 2H), 0.08 (s, 6H).

To a solution of 13-2 (1.4 g, 6.18 mmol, 1 eq) in MeOH (14 mL) was added p-TsOH (1.28 g, 7.42 mmol, 1.2 eq). The mixture was stirred at 30° C. for 12 hr to give a yellow solution. TLC (PE:EtOAc=10:1) showed the mixture was completed. The mixture was added H₂O (10 mL), extracted with EtOAc (15 mL*3). The organic layers were dried over sodium sulfate, filtered and concentrated under vacuum. The residue was purified by column chromatography (SiO₂, Petroleum ether/Ethyl acetate=100/0 to 95/5) to give 13-3. ¹H NMR (400 MHz, CDCl₃) δ ppm 4.27 (s, 2H) 1.79 (s, 1H) 0.99 (t, J=8.0 Hz, 3H) 0.65-0.55 (m, 2H) 0.17-0.10 (m, 6H).

To a solution of 13-3 (440 mg, 3.09 mmol, 1 eq) in acetone (4 mL) was added Jones Reagent (612.58 mg, 3.09 mmol, 1 eq) at 0° C. The mixture was stirred at 25° C. for 12 hr to give a black solution. TLC (PE:EtOAc=5:1) showed the mixture was completed. The mixture was diluted with MTBE (8 mL) and concentrated under vacuum. Then it was diluted with MTBE (5 mL) and H₂O (5 mL), extracted with MTBE (5 ml*3). The organic layers were dried over sodium sulfate, filtered and concentrated under vacuum. 13-4 was used next step without further purification. ¹H NMR (400 MHz, CDCl₃) δ ppm 1.05-0.99 (m, 3H) 0.73-0.64 (m, 2H) 0.23 (s, 6H).

To a solution of 13-4 (15.81 mg, 101.20 μmol, 0.9 eq) in DCM (3 mL) was added 2-chloro-1-methyl-pyridin-1-ium; iodide (43.09 mg, 168.67 μmol, 1.5 eq). The mixture was stirred at 25° C. for 1 hr to give a yellow suspension. The result mixture was dropwise to a solution of (1s,3R)—N-(4-((1S,3S)-3-butyl-6-methoxy-1,2,3,4-tetrahydroisoquinolin-1-yl)phenyl)adamantan-1-amine (50 mg, 112.45 μmol, 1 eq) and TEA (17.07 mg, 168.67 μmol, 23.48 uL, 1.5 eq) in DCM (3 mL) at 0° C. It was stirred at 0° C. for 2 hr to give a yellow solution. LCMS and TLC (PE:EtOAc=0/1, 3/1) showed (1s,3R)—N-(4-((1S,3S)-3-butyl-6-methoxy-1,2,3,4-tetrahydroisoquinolin-1-yl)phenyl)adamantan-1-amine remained and desired mass found. The mixture was quenched with H₂O (8 mL), extracted with DCM (8 mL*3). The organic layers were washed with HCl (1N, 10 mL). The organic layer was separated. The organic layers were washed with Sat·NaHCO₃ (15 mL). The organic layer was separated and dried over sodium sulfate, filtered and concentrated under vacuum. The residue was purified by column chromatography (SiO₂, Petroleum ether/Ethyl acetate=100/0 to 80/20) to give 1-((1S,3S)-1-(4-(((1s,3R)-adamantan-1-yl)amino)phenyl)-3-butyl-6-methoxy-3,4-dihydroisoquinolin-2(1H)-yl)-3-(ethyldimethylsilyl)prop-2-yn-1-one (33). LC-MS (m/z): 583.3 [M+H]⁺. ¹H NMR (400 MHz, CDCl₃) δ ppm 7.33-7.28 (m, 1H) 7.23 (br d, J=8.0 Hz, 1H) 6.92 (br d, J=6.8 Hz, 2H) 6.86-6.76 (m, 1H) 6.71-6.60 (m, 2H) 6.25 (br d, J=14.8 Hz, 1H) 4.56 (br s, 1H) 3.81 (s, 3H) 3.17-2.81 (m, 1H) 2.76-2.58 (m, 1H) 2.07 (br s, 3H) 1.75-1.58 (m, 12H) 1.32-1.16 (m, 6H) 1.04 (br t, J=7.6 Hz, 2H) 0.89-0.82 (m, 4H) 0.74-0.46 (m, 2H) 0.23 (s, 3H) 0.06 (br d, J=6.0 Hz, 3H).

### Procedure 14: Compound A-38

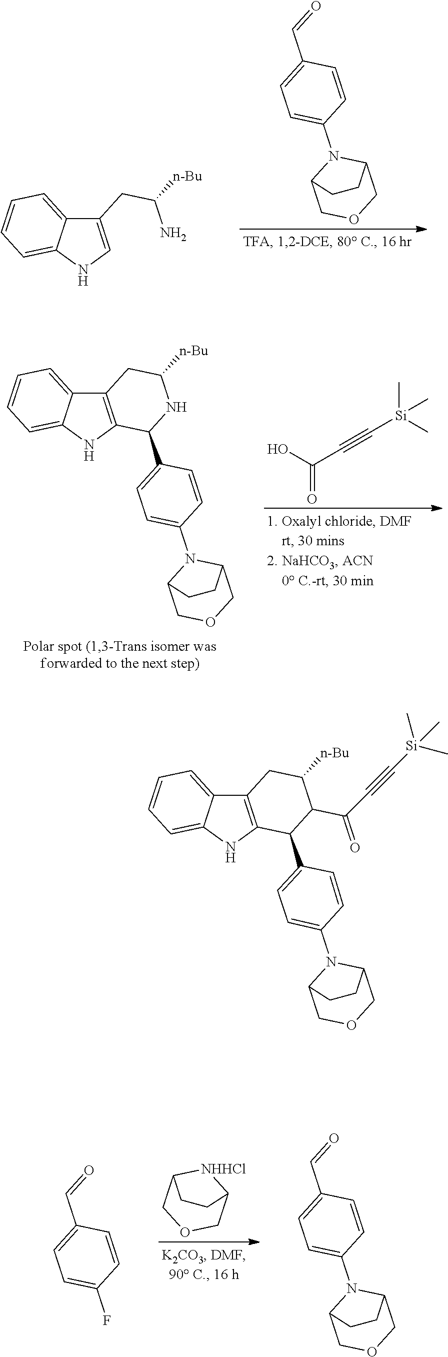

To a stirred solution of (1R,5S)-3-oxa-8-azabicyclo[3.2.1]octane hydrochloride (1.57 g, 10.5 mmol, 1.3 equiv.) DMSO (20.0 mL) was added potassium carbonate (5.57 g, 40.3 mmol, 5.0 equiv) at room temperature followed by the addition of 4-fluorobenzaldehyde (1.0 g, 8.06 mmol, 1.0 equiv) and then the reaction mixture was heated to 90° C. and stirred for 16 h. TLC (40% EtOAc in hexane) showed that the reaction was complete after this time. The reaction mixture was then cooled to room temperature, diluted with EtOAc (150 mL) and washed with water (20.0 mL). Organic layer was dried over anhydrous sodium sulfate, filtered and concentrated under reduced pressure to get the crude. The crude was purified by silica gel column chromatography using 20-25% EtOAc in hexane as an eluent to afford 4-(3-oxa-8-azabicyclo[3.2.1]octan-8-yl)benzaldehyde. LC-MS (ES) m/z: 218.1 [M+H]⁺

¹H NMR (400 MHz, DMSO-d6) δ ppm 1.91-1.99 (m, 4H), 3.46 (d, J=10.8 Hz, 2H), 3.61 (d, J=10.8 Hz, 2H), 4.37 (s, 2H), 6.93 (d, J=8.4 Hz, 2H), 7.67 (d, J=8.8 Hz, 2H), 9.66 (s, 1H).

To a solution of (2S)-1-(1H-indol-3-yl)hexan-2-amine (0.5 g, 2.31 mmol, 1.0 equiv) in 1,2-dichloroethane (5.0 mL) taken in a sealed tube was added 4-{3-oxa-8-azabicyclo[3.2.1]octan-8-yl}benzaldehyde (452 mg, 2.08 mmol, 0.9 equiv) at room temperature followed by the addition of TFA (0.36 mL, 4.62 mmol, 2.0 equiv.) and then the reaction was stirred at 80° C. for 10 h. TLC (5% MeOH/DCM) showed that the reaction was completed after 10 h. The reaction mixture was quenched with saturated NaHCO₃ solution and extracted into EtOAc (1×20 mL). Combined organic layer was dried over anhydrous Na₂SO₄, filtered and concentrated under reduced pressure to get the crude. Obtained crude product was purified by silica gel flash column chromatography MeOH/DCM 1-2% as eluent to give 8-{4-[(1S,3S)-3-butyl-1H,2H,3H,4H,9H-pyrido[3,4-b]indol-1-yl]phenyl}-3-oxa-8-azabicyclo[3.2.1]octane. LC-MS (ES) m/z: 416.2 [M+H]⁺

¹H NMR (400 MHz, DMSO-d6) δ ppm 0.81-0.91 (m, 4H), 1.16-1.89 (m, 11H), 2.25-2.31 (m, 1H), 2.65-2.78 (m, 2H), 3.06-3.48 (m, 3H), 3.55-4.06 (m, 2H), 4.16 (s, 2H), 5.01 (s, 1H), 6.75 (d, J=8.4 Hz, 1H), 6.90-7.00 (m, 4H), 7.21 (d, J=8.0 Hz, 1H), 7.38 (d, J=8.0 Hz, 1H), 10.64 (s, 1H).

To a stirred solution of 3-(trimethylsilyl)propiolic acid (0.103 g, 0.724 mmol, 1.0 equiv) in DMF (0.002 mL, 0.002 mmol, 0.04 equiv) was added oxalyl chloride (0.063 mL, 0.796 mmol, 1.1 equiv), was added at room temperature and stirred for 30 min. After completion, the reaction mass was concentrated under nitrogen atmosphere and taken to next step.

To a stirred solution of 8-{4-[(1S,3S)-3-butyl-1H,2H,3H,4H,9H-pyrido[3,4-b]indol-1-yl]phenyl}-3-oxa-8-azabicyclo[3.2.1]octane (100 mg, 241 μmol, 1.0 equiv.) in ACN (5.0 mL) was added sodium bicarbonate (0.141 g, 1.68 mmol, 7.0 equiv.) at 0° C., stirred at 0° C. for 15 mins and then 3-(trimethylsilyl)propioloyl chloride (0.090 g, 0.722 mmol, 2.0 equiv.) in ACN (2.0 mL) was added at 0° C. and the reaction was stirred at room temperature for 30 mins. Reaction was monitored by TLC (70% EtOAc in hexane). After this time the reaction mixture was diluted with EtOAc (100 mL) and was washed with water (10 mL). Organic layer was dried over anhydrous sodium sulfate, filtered and concentrated under reduced pressure to afford 1-[(1S,3S)-3-butyl-1-(4-{3-oxa-8-azabicyclo[3.2.1]octan-8-yl}phenyl)-1H,2H,3H,4H,9H-pyrido[3,4-b]indol-2-yl]-3-(trimethylsilyl)prop-2-yn-1-one. LC-MS (ES) m/z: 540.3 [M+H]⁺.

### Procedure 15—Synthesis of Compound A-39

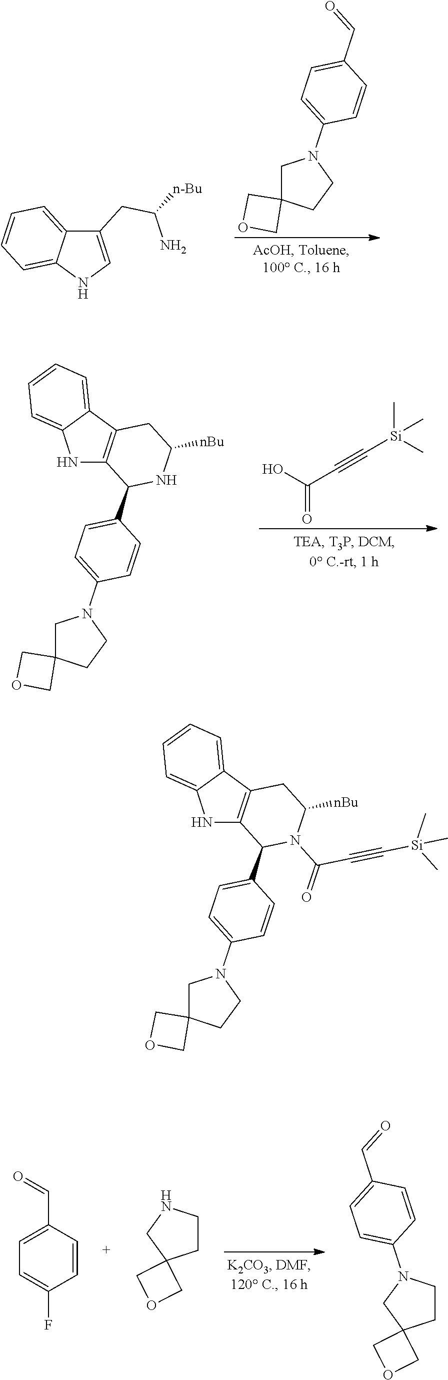

To a stirred solution of 2-oxa-6-azaspiro[3.4]octane (1.19 g, 10.5 mmol, 1.3 equiv) in DMF (15.0 mL) was added potassium carbonate (2.26 g, 16.1 mmol, 2.0 equiv) at room temperature followed by the addition of 4-fluorobenzaldehyde 66-A (1.0 g, 8.06 mmol, 1.0 equiv) and then the reaction mixture was heated to 120° C. and stirred for 16 h. TLC (40% EtOAc in hexane) showed that the reaction was complete after this time. The reaction mixture was then cooled to room temperature and diluted with ice and then extracted with EtOAc (50 mL). Organic layer was dried over anhydrous sodium sulfate, filtered and concentrated under reduced pressure to get the crude. The crude was purified by silica gel column chromatography using 20-25% EtOAc in hexane as an eluent to afford 4-{2-oxa-6-azaspiro[3.4]octan-6-yl}benzaldehyde. LC-MS (ES) m/z: 218.1 [M+H]⁺. ¹H NMR (400 MHz, DMSO-d6): δ ppm 2.25-2.28 (m, 2H), 3.29-3.37 (m, 2H), 3.60 (s, 2H), 4.50-4.58 (m, 4H), 6.62 (d, J=8.4 Hz, 2H), 7.66 (d, J=8.8 Hz, 2H), 9.64 (s, 1H).

To a solution of (2S)-1-(1H-indol-3-yl)hexan-2-amine (850 mg, 3.93 mmol, 1.0 equiv.) in Toluene (10.0 mL) take in sealed tube was added 4-{2-oxa-6-azaspiro[3.4]octan-6-yl}benzaldehyde (1.11 g, 1.3 equiv, 5.11 mmol) at room temperature followed by the addition of acetic acid (0.67 ml, 3 equiv, 7.86 mmol) and then the reaction was stirred at 100° C. for 16 hr. Reaction mixture was cooled to room temperature and evaporated under reduced pressure. obtained crude was quenched with saturated sodium bicarbonate solution then extracted with ethyl acetate (2×30 mL). Combined organic layer was washed with brine, dried over anhydrous sodium sulphate. Organic layer was filtered and concentrated under reduced pressure to get crude product. Crude was purified by flash column chromatography using ethyl acetate in hexane as eluent. Product was isolated at 20-22% ethyl acetate in hexane. Product fractions collected and concentrated under reduced pressure to get 6-{4-[(1S,3S)-3-butyl-1H,2H,3H,4H,9H-pyrido[3,4-b]indol-1-yl]phenyl}-2-oxa-6-azaspiro[3.4]octane. LC-MS (ES) m/z: 416.3 [M+H]L.

To a stirred solution of 3-(trimethylsilyl)prop-2-ynoic acid (0.103 g, 0.722 mmol, 1.2 equiv) in DCM (8.0 mL) at 0° C. was added triethylamine (0.254 mL, 1.80 mmol, 3.0 equiv) followed by propanephosphonic acid anhydride (T3P) (50 wt. % in EA, 1.1 mL, 0.902 mmol, 1.5 equiv). After stirring for 5 minutes 6-{4-[(1S,3S)-3-butyl-1H,2H,3H,4H,9H-pyrido[3,4-b]indol-1-yl]phenyl}-2-oxa-6-azaspiro[3.4]octane (250 mg, 0.602 μmol, 1.0 equiv.) was added. Then reaction was stirred at room temperature for 1 h. Reaction was monitored by TLC (25% EtOAc in hexane). After this time the reaction mixture was diluted with EtOAc (25 mL) and was washed with water (5 mL). Organic layer was dried over anhydrous sodium sulfate, filtered and concentrated under reduced pressure to obtain 1-[(1S,3S)-3-butyl-1-(4-{2-oxa-6-azaspiro[3.4]octan-6-yl}phenyl)-1H,2H,3H,4H,9H-pyrido[3,4-b]indol-2-yl]-3-(trimethylsilyl)prop-2-yn-1-one.

### Procedure 16—Preparation of Compound A-40

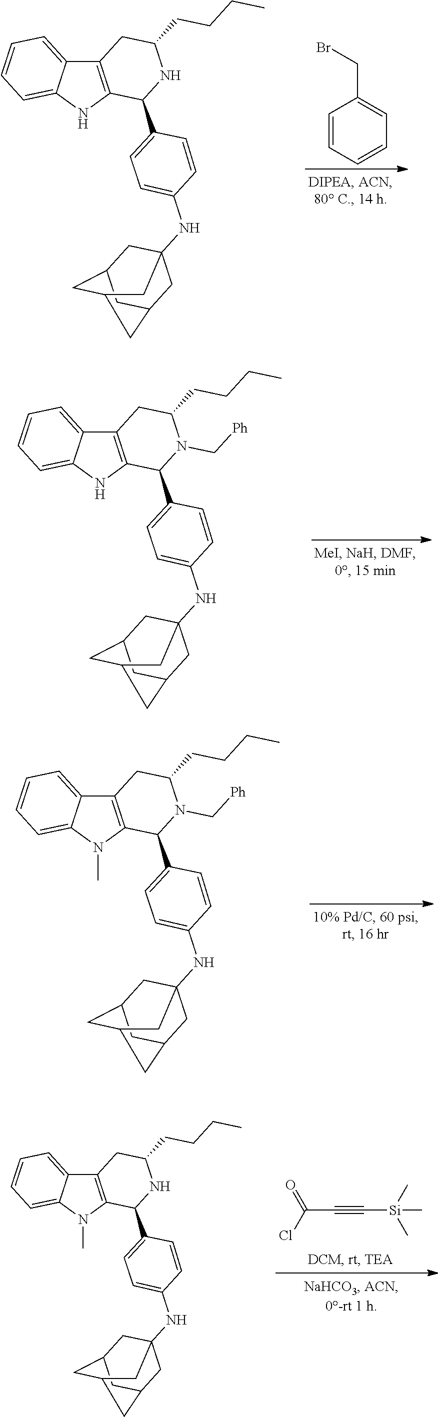

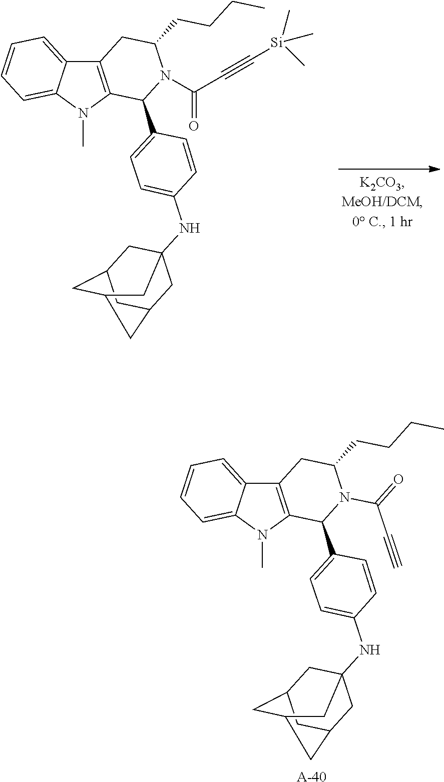

To a solution of N-{4-[(1S,3S)-3-butyl-1H,2H,3H,4H,9H-pyrido[3,4-b]indol-1-yl]phenyl}adamantan-1-amine (400 mg, 882 μmol, 1 equiv) in acetonitrile (5.0 mL) was added DIPEA (462 μL, 2.65 mmol, 3 equiv) and (bromomethyl)benzene (209 μL, 1.76 mmol, 2 equiv) at room temperature. This reaction was stirred at 80° C. for 14 h. TLC (8% EtOAc in hexane) showed the reaction was completed. The reaction was cooled to room temperature and was concentrated under reduced pressure to get the crude product. This crude product was taken to next step. LCMS (ES) m/z=544.2 [M+H]⁺

To a stirred solution of tert-butyl (1S,3S)-1-{4-[(adamantan-1-yl)amino]phenyl}-3-butyl-1H,2H,3H,4H,9H-pyrido[3,4-b]indole-2-carboxylate (0.500 g, 0.919 mmol, 1.0 equiv) in DMF (5 mL) at 0° C. was added sodium hydride (60% in mineral oil, 0.044 g, 1.08 mmol, 1.2 equiv). Then reaction mixture was stirred at same temperature for 15 minutes, then methyl iodide (0.090 mL, 1.35 mmol, 1.5 equiv) added. Then reaction mixture was allowed to stir at room temperature for 30 minutes. Then reaction mixture was quenched with ice water, extracted with ethyl acetate (2×15 mL). Combined organic layer was washed with brine (5 mL), dried over anhydrous sodium sulphate. Organic layer was filtered and concentrated under reduced pressure to get crude N-{4-[(1S,3S)-2-benzyl-3-butyl-9-methyl-1H,2H,3H,4H,9H-pyrido[3,4-b]indol-1-yl]phenyl}adamantan-1-amine.

In a par hydrogenation vessel to a solution of N-{4-[(1S,3S)-2-benzyl-3-butyl-9-methyl-1H,2H,3H,4H,9H-pyrido[3,4-b]indol-1-yl]phenyl}adamantan-1-amine (0.500 g, 0.896 mmol, 1.0 equiv) in methanol (20 mL) at rt, 10% palladium on carbon (50 mg) was added. Then reaction mixture was stirred under hydrogen at 60 psi for 16 h. Then reaction mixture was filtered through celite bed. Celite bed was washed with methanol. Organic layer was filtered and concentrated under reduced pressure. Obtained crude N-{4-[(1S,3S)-3-butyl-9-methyl-1H,2H,3H,4H,9H-pyrido[3,4-b]indol-1-yl]phenyl}adamantan-1-amine was taken forward without further purification. LC-MS(ES) m/z: 468.4 [M+H]⁺

To a stirred solution of N-{4-[(1S,3S)-3-butyl-9-methyl-1H,2H,3H,4H,9H-pyrido[3,4-b]indol-1-yl]phenyl}adamantan-1-amine (0.210 g, 0.449 mmol, 1.0 equiv) in acetonitrile (10.0 mL) at 0° C. was added sodium bicarbonate (0.302 g, 3.59 mmol, 8.0 equiv) in acetonitrile (2 mL) at 0° C. After stirring for 5 minutes 3-(trimethylsilyl)prop-2-ynoyl chloride (0.072 g, 0.449 mmol, 1.2 equiv) was added at same temperature. Then reaction mixture was allowed to stirred at room temperature for 15 minutes. Then reaction mixture was diluted with water (5 mL), extracted with ethyl acetate (2×10 mL). Combined organic layer was washed with brine (5 mL), dried over anhydrous sodium sulphate. Organic layer was filtered and concentrated under reduced pressure to obtain 1-((1S,3S)-1-(4-(((1R,3R,5S)-adamantan-1-yl)amino)phenyl)-3-butyl-9-methyl-1,3,4,9-tetrahydro-2H-pyrido[3,4-b]indol-2-yl)-3-(trimethylsilyl)prop-2-yn-1-one. LC-MS(ES) m/z: 592.3 [M+H]⁺

Compounds disclosed herein, can be or were, synthesized according to the procedures described above using the appropriate reagents and starting materials. Select data are shown in Tables A-2 and A-3.

TABLE A-2
Characterization details of selected compounds.

| | | MS |
|---|---|---|
| No. | Structure | [M + H]⁺ |
| A-1 | 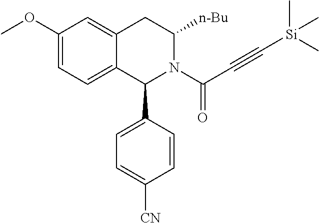 | 445 |
| A-2 | 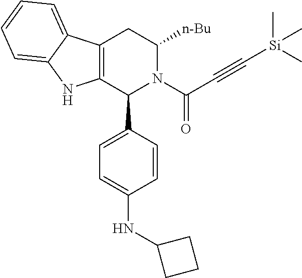 | 498.3 |
| A-3 | 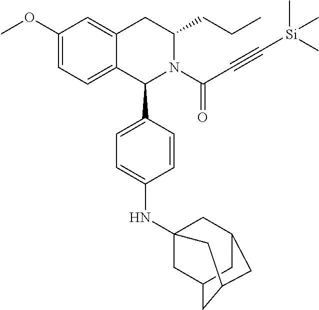 | 555.5 |
| A-4 | 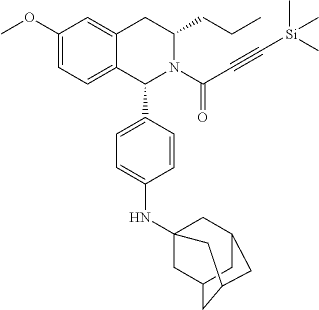 | 555.3 |
| A-6 | 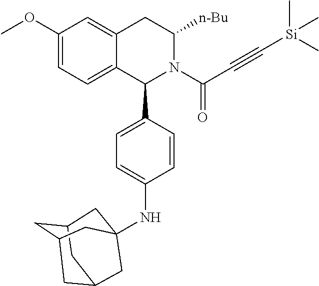 | 569.3 |
| A-7 | 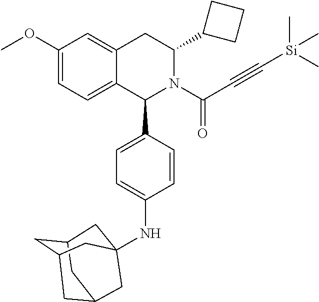 | 567.3 |
| A-8 | 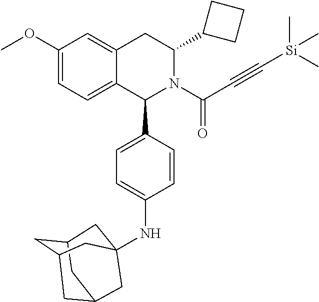 | 567.2 |
| A-9 | 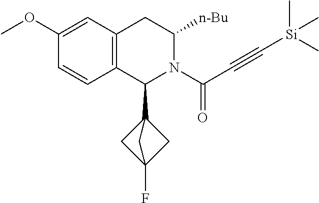 | 428.2 |
| A-10 | 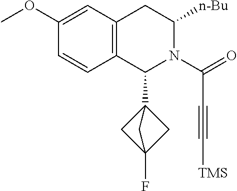 | 428.2 |
| A-11 | 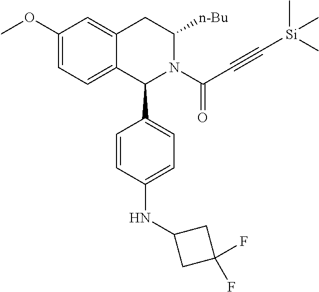 | 525.3 |
| A-12 | 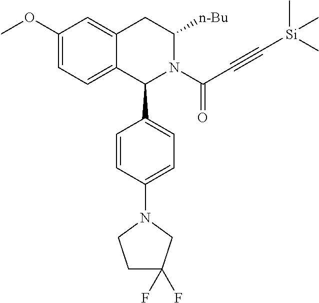 | 525.2 |
| A-15 | 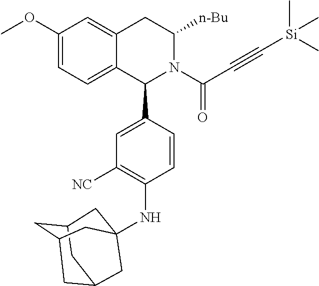 | 594.2 |
| A-16 | 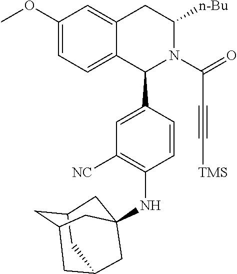 | 594.5 |
| A-28 | 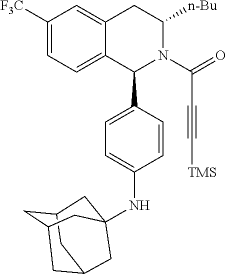 | 607.1 |
| A-29 | 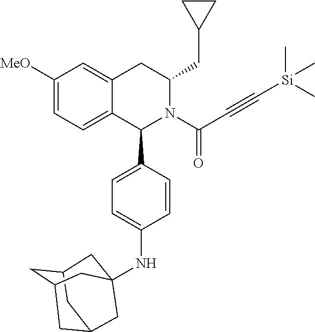 [SMILES: COc1ccc2c(c1)C[C@H](CC1CC1)N(C(=O)C#C[Si](C)(C)C)[C@H]2c1ccc(NC23CC4CC(CC(C4)C2)C3)cc1] | 495.3 |
| A-33 | 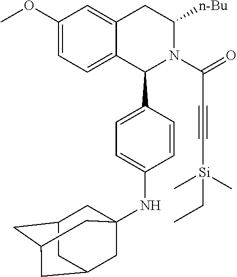 | 583.3 |
| A-41 | 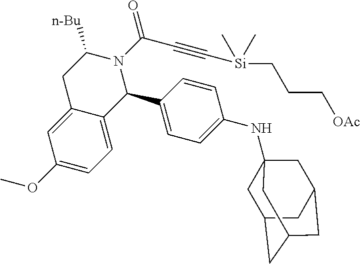 | 655.4 |
| A-42 | 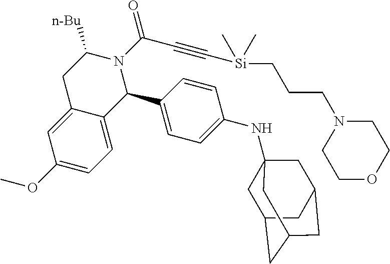 | 682.4 |
| A-43 | 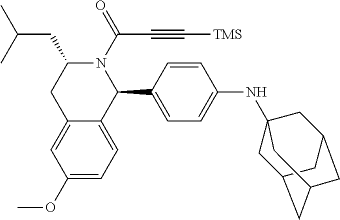 [SMILES: COc1ccc2c(c1)C[C@H](CC(C)C)N(C(=O)C#C[Si](C)(C)C)[C@H]2c1ccc(NC23CC4CC(CC(C4)C2)C3)cc1] | 569.1 |
| A-44 | 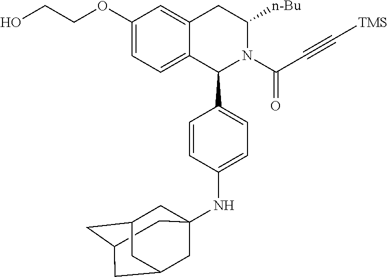 | 527.3 |
| A-45 | 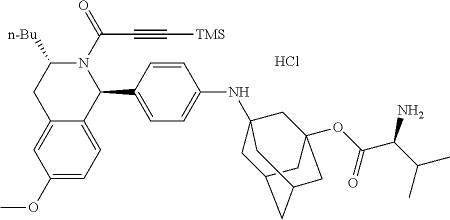 | 684.4 |
| A-46 | 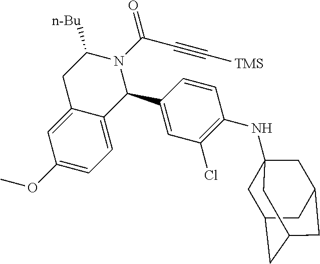 | 603.3 |
| A-47 | 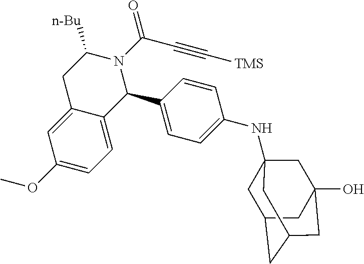 | 585.7 |
| A-51 | 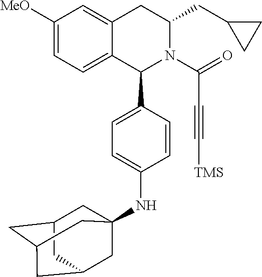 [SMILES: COc1ccc2c(c1)C[C@H](CC1CC1)N(C(=O)C#C[Si](C)(C)C)[C@H]2c1ccc(N[C@]23CC4CC(C[C@H](C4)C2)C3)cc1] | 567.3 |
| A-57 | 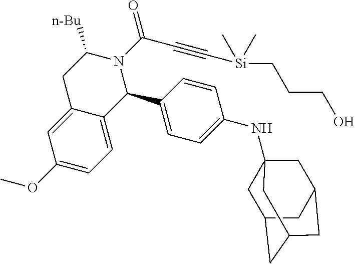 | 635.4 |

### Intermediate Procedure B-1: Synthetic scheme for tert-butyl (S)-(1-(3-methoxyphenyl)hexan-2-yl)carbamate

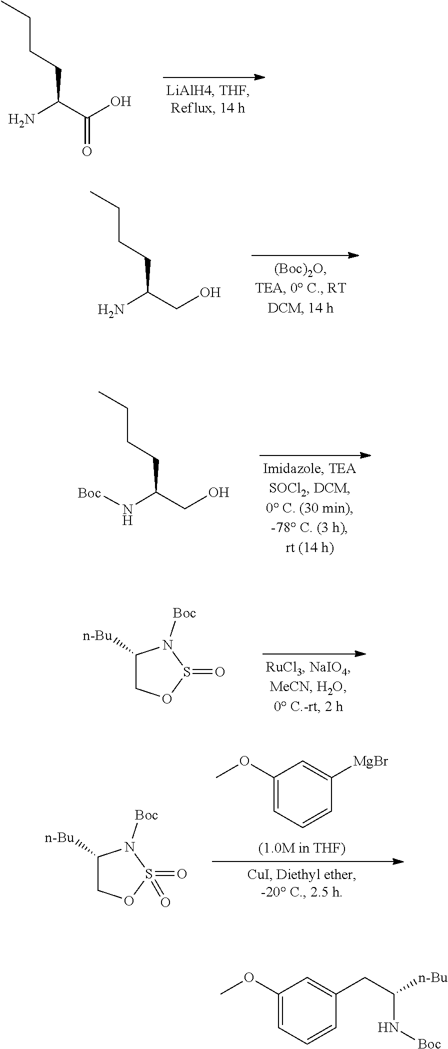

To a solution of (S)-2-aminohexanoic acid (30.0 g, 228.6 mmol, 1 eq) in THF (300 mL) at 0° C. was added lithium aluminumhydride (1 M in THF, 458 mL, 457.3 mmol, 2 eq) over a period of 1 h. Reaction mixture was warm to room temperature, then the mixture was stirred at 70° C. for 14 h under N2 atmosphere. Reaction mixture was cooled to room temperature, the reaction was diluted with diethyl ether (50 mL), after fisher—workup, reaction mixture was filtered through sintered funnel, using diethyl ether, filtrate was concentrated under reduced pressure to get the product, without further purification crude product was forward to next step. ¹H NMR (400 MHz, CDCl3) δ ppm 0.89 (s, 3H), 1.29-1.39 (m, 6H), 2.00 (s, 3H), 2.81 (s, 1H), 3.23-3.25 (m, 1H), 3.55-3.56 (m, 1H).

To a solution of (S)-2-aminohexan-1-ol (24.5 g, 209.06 mmol, 1 eq) in DCM (250 mL) was added TEA (58.76 mL, 418.12 mmol, 2 eq) at 0° C. drop wise, it was stirred for 5 mins, then di-tert-butyl dicarbonate (57.63 mL, 250.87 mmol, 1.2 eq). After stirring at room temperature for 14 h, diluted with water (30 mL), extracted with DCM (2×150 mL). Combined organic layer was washed with water, then with aq NaHCO₃ solution (˜30 mL) and finally with brine solution (75 mL), dried over Na₂SO₄, and concentrated in-vacuo. The residue was subjected to Combiflash silica gel chromatography equipped MeOH in DCM as an eluent to give the tert-butyl (S)-(1-hydroxyhexan-2-yl)carbamate. ¹H NMR (400 MHz, CDCl3) δ ppm 0.90 (s, 3H), 1.25-1.33 (m, 6H), 1.38-1.41 (m, 9H), 3.48-3.55 (m, 1H), 3.62-3.68 (m, 2H), 4.57 (bs, 1H).

1H-imidazole (25 g, 368.1 mmol, 4 equiv) and triethlamine (39 mL, 276.1 mmol, 3 eq) were dissolved in anhydrous dichloromethane (200 mL, commercial dry solvent) at rt and the mixture was cooled to 0° C. (external temp, maintained with ice). Then thionyl chloride (7.3 mL, 101.2 mmol, 1.1 eq) was added slowly dropwise through an additional funnel over a period of −30 minutes while maintaining the bath temperature at 0° C. The reaction mixture was then stirred for additional 20 mins at 0° C. Then the reaction mixture was cooled −78° C. Then a solution of tert-butyl (S)-(1-hydroxyhexan-2-yl)carbamate (20 g, 92.03 mmol, 1 eq) made in anhydrous dichloromethane (100 mL, commercial dry solvent) at rt was added through an additional funnel dropwise to the reaction mixture stirred at −78° C. over a period of 45 mins. The reaction mixture was stirred at −78° C. for additional 3 hours. Then the dry ice-acetone bath was removed and the reaction mixture was allowed to stir at room temperature for 16 h. After completion of the reaction (TLC, 10% EA in hexane) the mixture was diluted with DCM, washed with water (200 mL×3) and brine (200 mL). The organic phase was dried over anhydrous Na₂SO₄, filtered and concentrated under reduced pressure at rotavapor to get the crude. The crude was purified by silica gel column chromatography using ethyl acetate in hexane as eluent. Product was eluted at 10-25% of EA in hexane to give the tert-butyl (4S)-4-butyl-1,2,3-oxathiazolidine-3-carboxylate 2-oxide. Note: This reaction was performed 20 g×2 batches. ¹H NMR (400 MHz, CDCl3) δ ppm 0.91 (t, J=6.8 Hz, 3H), 1.27-1.38 (m, 4H), 1.52 (s, 9H), 1.67-1.73 (m, 1H), 1.99-2.10 (m, 1H), 3.97-4.02 (m, 1H), 4.70-4.78 (m, 2H).

Ruthenium(III)chloride (0.463 g, 2.23 mmol, 0.014 eq), was added to a stirred solution of tert-butyl (4S)-4-butyl-1,2,3-oxathiazolidine-3-carboxylate 2-oxide (42.0 g, 159.48 mmol, 1 eq), in acetonitrile (400 mL) and water (200 mL) at 0° C., followed by portion wise addition of sodium metaperiodate (37.43 g, 175.43 mmol, 1.1 eq). The biphasic mixture was stirred at rt for 2 hours. Reaction mixture was filtered through sintered, washed with ethyl acetate. Water (250 mL) was added and the mixture was extracted in to ethyl acetate (2×150 mL). The combined organics were washed with water (150 mL), brine (150 mL), dried over with Na₂SO₄, filtered and concentrated under reduced pressure to get the crude product, crude product was purified by column chromatography using 10% ethylacetate in Hexane as an eluent to give the tert-butyl (S)-4-butyl-1,2,3-oxathiazolidine-3-carboxylate 2,2-dioxide. ¹H NMR (400 MHz, CDCl₃) δ ppm 0.89-0.92 (m, 3H), 1.24-1.37 (m, 4H), 1.53 (s, 9H), 1.77-1.83 (m, 1H), 1.88-1.89 (m, 1H), 4.26-4.30 (m, 2H), 4.59-4.63 (m, 1H).

To a stirred solution of cuprous iodide (3.27 g, 17.2 mmol, 0.2 eq) in tetrahydrofuran (180 mL) was added 1 M solution of (3-methoxyphenyl)magnesium bromide (258 mL, 258 mmol, 3.0 eq) in tetrahydrofuran drop wise over a period of 15 min at −20° C., stirred for 30 min at same temperature. A solution of tert-butyl (S)-4-butyl-1,2,3-oxathiazolidine-3-carboxylate 2,2-dioxide (24.0 g, 85.9 mmol, 1.0 eq) in tetrahydrofuran (70 mL) was added drop wise at −20° C. and the reaction mixture was allowed to stir for 2.5 h at −20° C. The reaction was monitored by TLC. After completion, the reaction mixture was quenched with 10% aqueous citric acid solution at −20° C., allowed to warm to room temperature and stirred for 15 min. The mixture was filtered through celite pad and washed with ethyl acetate. The ethyl acetate layer was separated from the filtrate, washed with water and brine solution, dried over sodium sulphate, filtered and concentrated to obtained crude product, which was purified by column chromatography using gradient of 0-10% ethyl acetate in hexane over 230-400 mesh silica gel. The desired fractions were concentrated to afford tert-butyl (S)-(1-(3-methoxyphenyl)hexan-2-yl)carbamate. LC-MS(ES)m/z: 308.2 [M+H]+ but observed 252.1 without tert-butyl group. ¹H NMR (400 MHz, CDCl3) δ ppm: 0.86-0.88 (m, 3H), 1.27-1.46 (m, 15H), 2.73 (s, 2H), 3.79 (s, 4H), 4.29 (s, 1H), 6.72-6.77 (m, 3H), 7.17-7.26 (m, 1H).

### Intermediate Procedure B-2: Synthesis of 4-(((3s,5s,7s)-adamantan-1-yl)amino)benzoic Acid

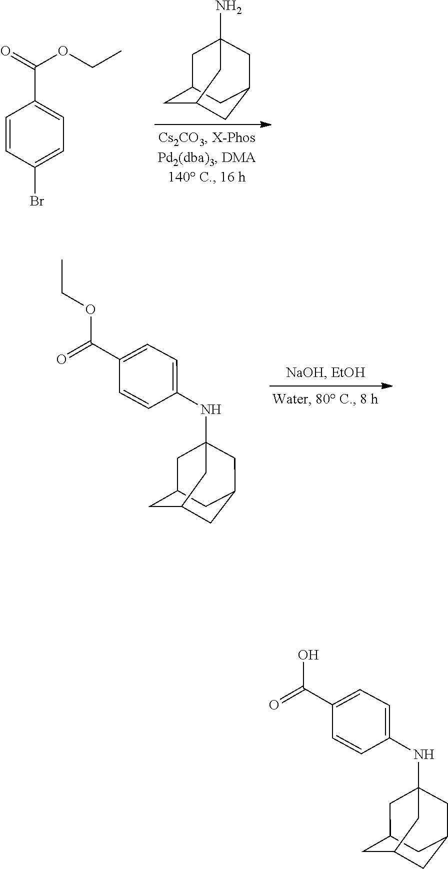 [SMILES: CCOC(=O)c1ccc(Br)cc1 ; NC12CC3CC(CC(C3)C1)C2 ; CCOC(=O)c1ccc(NC23CC4CC(CC(C4)C2)C3)cc1 ; O=C(O)c1ccc(NC23CC4CC(CC(C4)C2)C3)cc1]

To a stirred solution of ethyl 4-bromobenzoate (40.0 g, 175 mmol, 1.0 equiv) in Dimethylacetamide (250 mL) were added adamantan-1-amine (39.6 g, 262 mmol, 1.5 equiv), cesium carbonate (114 g, 349 mmol, 2.0 equiv) at room temperature under nitrogen atmosphere and then purged with nitrogen for 10 min and Tris(dibenzylideneacetone)dipalladium(0) (8.0 g, 8.73 mmol, 0.05 equiv) and 2-Dicyclohexylphosphino-2′,4′,6′-triisopropylbiphenyl (8.32 g, 17.5 mmol, 0.1 equiv) were added. The reaction mixture was heated at 140° C. for 16 h. After completion of the reaction, the reaction mixture was filtered through celite pad and washed with diethyl ether (500 mL). The filtrate was washed with ice cooled water (200 mL) and brine solution (100 mL). The organic layer was dried over anhydrous sodium sulphate, filtered and concentrated to obtained crude product (60.0 g). The crude was purified by column chromatography using gradient 0-10% ethyl acetate in hexane. The desired fractions were concentrated to afford ethyl 4-[(adamantan-1-yl)amino]benzoate.

The above mixture (20.5 g) was dissolved in diethyl ether (102 ml, 5 volume) and 2N HCl (205 ml, 10 volume) was added and mixture was concentrated to remove diethyl ether, filtered the solid, washed with diethyl ether. The resulting solid was basified with sodium carbonate solution, extracted the desired product with ethyl acetate. The ethyl acetate layer was washed with brine solution, dried over sodium sulfate, and concentrated under reduced pressure to afford ethyl 4-[(adamantan-1-yl)amino]benzoate. LC-MS(ES)m/z: 300.2 [M+H]+ complies. ¹H NMR (400 MHz, CDCl3) δ: 1.22-1.36 (m, 3H), 1.71 (s, 6H), 1.97 (s, 6H), 2.14 (s, 3H), 4.28-4.33 (m, 2H), 6.69 (d, J 8.4 Hz, 2H), 7.81 (d, J=8.8 Hz, 2H).

To a stirred solution of ethyl 4-[(adamantan-1-yl)amino]benzoate (13.5 g, 45.1 mmol, 1.0 equiv) in ethanol (200 mL), was added 1 M sodium hydroxide solution (90.2 mL, 2 equiv, 90.2 mmol) at room temperature and heated to 80° C., stirred for 8 h. After completion, the reaction mixture was concentrated under reduced pressure to remove ethanol from the reaction mass. The aqueous layer was acidified with 5% aqueous solution of citric acid up to pH=4, filtered the solid compound, washed with pentane, dried under vacuum to afford 4-[(adamantan-1-yl)amino]benzoic acid. ¹H NMR (400 MHz, DMSO) δ ppm 1.65 (s, 6H), 1.91 (s, 6H), 2.06 (s, 3H), 5.86 (s, 1H), 6.72 (d, J=8.8 Hz, 2H), 7.58 (d, J=8.8 Hz, 2H), 11.91 (s, 1H).

### Intermediate Procedure B-3: S)-1-(4-(((3R,5R,7R)-adamantan-1-yl)amino)phenyl)-2-benzyl-3-butyl-6-methoxy-3,4-dihydroisoquinolin-2-iumbromide

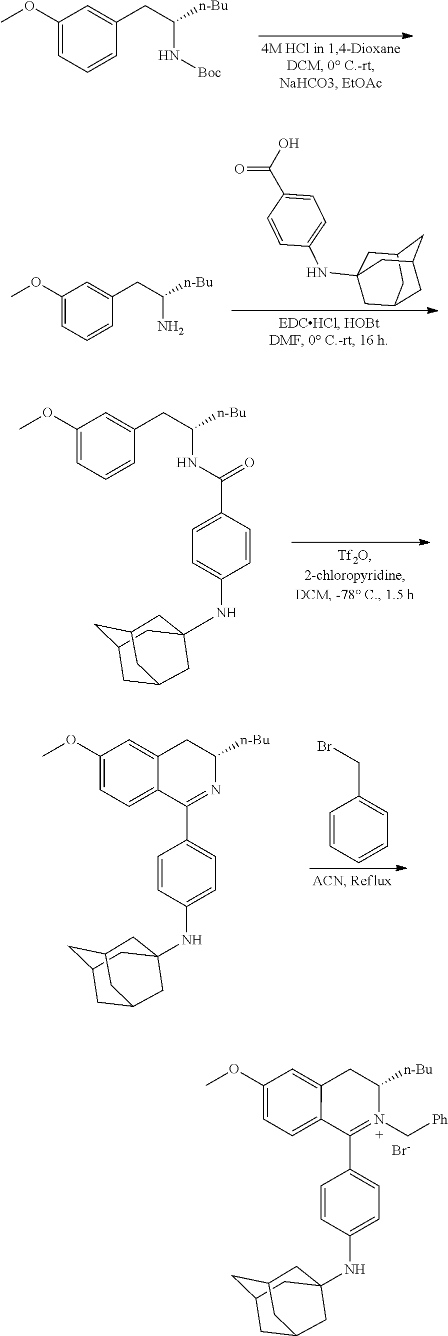

To a stirred solution of tert-butyl N-[(2S)-1-(3-methoxyphenyl)hexan-2-yl]carbamate (20.0 g, 65.1 mmol, 1 eq) in dichloromethane (200 mL), was added 4 M HCl in 1,4-dioxane (120 mL) at 0° C. and the reaction mixture was stirred at room temperature for 16 h. The progress of reaction was monitored by TLC. After completion, the reaction mixture was concentrated under reduced pressure and the residue part was basified with aqueous sodium bicarbonate solution (pH˜9) product was extracted with ethyl acetate (2×150 mL). Combined organic layer was washed with brine (30 mL), dried over anhydrous sodium sulphate, filtered and concentrated under the vacuum to afford (2S)-1-(3-methoxyphenyl)hexan-2-amine. LC-MS (m/z)=208.7 [M+H]⁺. 1H NMR (400 MHz, DMSO-d6) δ ppm 0.83-0.85 (m, 3H), 1.23-1.34 (m, 6H), 2.40-2.46 (m, 1H), 2.48-2.59 (m, 1H), 2.61-2.83 (m, 2H), 3.71 (s, 4H), 6.71-6.73 (m, 3H), 7.15-7.19 (m, 1H).

To a stirred solution of 4-[(adamantan-1-yl)amino]benzoic acid (8.51 g, 31.4 mmol, 1 eq) in DMF (60 mL) was added 1H-1,2,3-benzotriazol-1-ol (6.35 g, 47.0 mmol, 1.5 eq) and stirred for 5 minutes at 0° C., then EDC·HCl (9.02 g, 47 mmol, 1.5 eq) followed by (2S)-1-(3-methoxyphenyl)hexan-2-amine (6.5 g, 31.4 mmol, 1 eq) and ethylbis(propan-2-yl)amine (16.8 mL, 94.1 mmol, 3 eq) was added to reaction mixture, stirred at room temperature for 16 h. Progress of the reaction was monitored by TLC (30% EtOAc:n-Hexane). After completion of the reaction, the reaction mixture was diluted with ice-water and stirred for 5 minutes, precipitation was observed. The obtained solid was filtered through sintered funnel, washed with ethyl acetate and filtrate was further washed with water and brine, dried over anhydrous sodium sulphate. Organic layer was evaporated under the reduced pressure to obtain crude. The crude was purified by silica gel flash column chromatography using EtOAC:n-Hexane as an eluent 5-10%. Desired fractions were then evaporated under the vacuum to afford 4-[(adamantan-1-yl)amino]-N-[(2S)-1-(3-methoxyphenyl) hexan-2-yl]benzamide. LC-MS(ES)m/z: 461.7 [M+H]+ complies. 1H NMR (400 MHz, DMSO d6) δ ppm: 0.80-0.82 (m, 4H), 1.14-1.25 (m, 5H), 1.46 (d, J=5.6 Hz, 2H), 1.64 (s, 6H), 1.89 (s, 4H), 2.05 (s, 3H), 2.66-2.79 (m, 2H), 3.66 (s, 3H), 4.06-4.11 (m, 1H), 5.53 (s, 1H), 6.68-6.77 (m, 5H), 7.13 (t, J=7.6 Hz, 1H), 7.50 (d, J=8.4 Hz, 2H), 7.65 (d, J=8.4 Hz, 1H).

To stirred solution of 4-[(adamantan-1-yl)amino]-N-[(2S)-1-(3-methoxyphenyl) hexan-2-yl]benzamide (6 g, 13.0 mmol, 1 eq) and 2-chloropyridine (3.7 mL, 39.1 mmol, 3.0 eq) in dichloromethane (80 mL) was added trifluoromethanesulfonic anhydride (6.56 mL, 39.1 mmol, 3.0 eq) slowly drop wise at −78° C. for 5 min. Then the reaction mixture was cooled to 0° C. for 5 min, the resulting solution was allowed to warm to room temperature and allowed to stir for 2 h. Progress of the reaction was monitored by TLC (50% EtOAc in n-hexane). After the completion of the reaction, reaction mixture was quenched with 1N NaOH solution (pH maintained ˜14) at 0° C., stirred for 10 mins and extracted with DCM (2×100 mL). Combined organic layer was washed with water (100 mL) and brine (50 mL), dried over anhydrous sodium sulfate, filtered and concentrated under reduced pressure to get the crude. The crude was purified by silica gel flash column chromatography using EtOAc in n-hexane as an eluent in 35%-40%, desired fractions were evaporated under the vacuum to afford 4-[(3S)-3-butyl-6-methoxy-3,4-dihydroisoquinolin-1-yl]benzonitrile. LC-MS (ES) m/z: 443.6 [M+H]⁺. 1H NMR (400 MHz, DMSO-d6) δ ppm 0.89 (t, J=7.2 Hz, 3H), 1.16 (t, J=6.8 Hz, 2H), 1.22-1.34 (m, 3H), 1.65 (s, 9H), 1.91 (s, 3H), 2.06 (s, 3H), 2.31-2.37 (m, 2H), 2.59-2.72 (m, 1H), 3.21 (s, 1H), 3.78 (s, 2H), 4.01 (d, J=6.4 Hz, 1H), 5.74 (s, 1H), 6.75-6.88 (m, 4H), 7.24 (t, J=8.4 Hz, 3H).

To stirred solution of N-{4-[(3S)-3-butyl-6-methoxy-3,4-dihydroisoquinolin-1-yl]phenyl}adamantan-1-amine (7.80 g, 17.6 mmol, 1 eq) was added (bromomethyl)benzene (3.14 mL, 26.4 mmol, 1.5 eq) in acetonitrile (40.0 mL) was heated at 85° C. for 5 h. Progress of the reaction was monitored by 1H NMR & LC-MS. After completion of the reaction, reaction mixture was concentrated under reduced pressure to get the crude product. Obtained crude product was triturated with n-Pentane (2×25 mL) to get the desired product (3S)-1-{4-[(adamantan-1-yl)amino]phenyl}-2-benzyl-3-butyl-6-methoxy-3,4-dihydroisoquinolin-2-ium bromide. The resulting residue crude was used in the next reaction without any further purification. LC-MS (ES) (m/z)=533.4 [M+H]+ was observed along with 623.4 [M+H]+ debenzylated product.

### Intermediate Procedure B-4: Synthesis of (1S,3R,5S)—N-(4-((1R,3S)-3-(cyclopropylmethyl)-6-methoxy-1,2,3,4-tetrahydroisoquinolin-1-yl)phenyl)adamantan-1-amine and (1S,3R,5S)—N-(4-((1S,3S)-3-(cyclopropylmethyl)-6-methoxy-1,2,3,4-tetrahydroisoquinolin-1-yl)phenyl)adamantan-1-amine

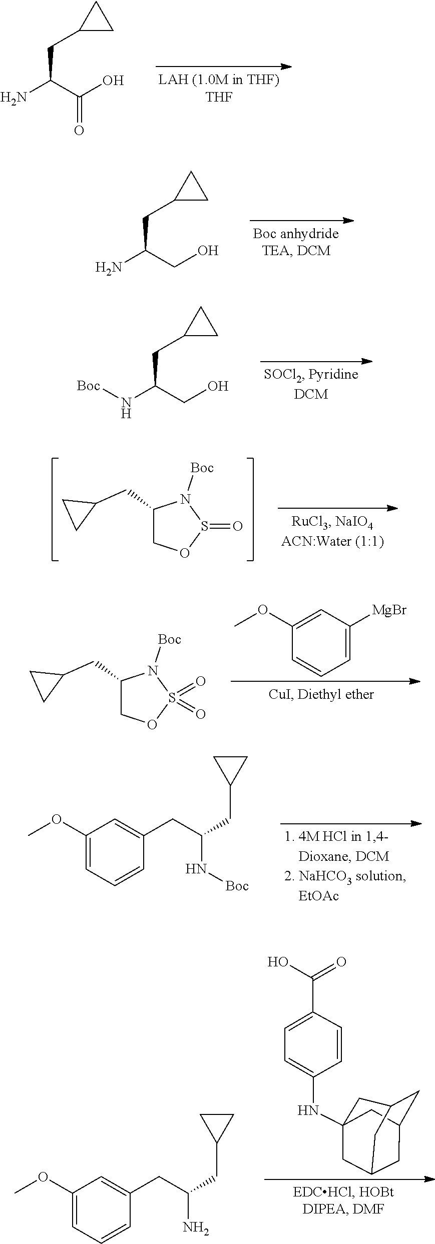

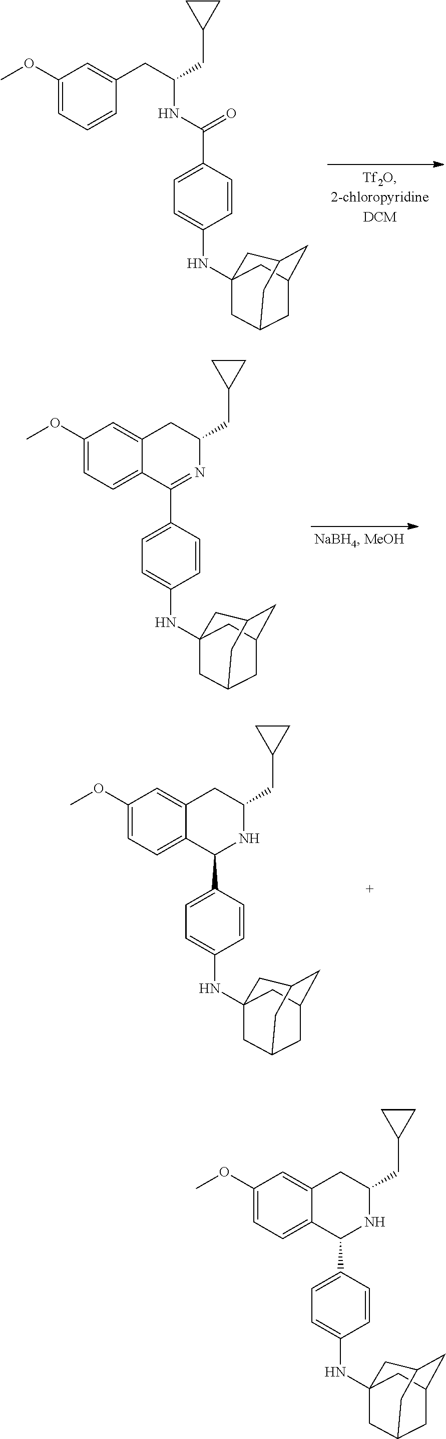

To a solution of (2S)-2-amino-3-cyclopropylpropanoic acid (5.0 g, 38.7 mmol, 1 eq) in THF (100.0 mL) at 0° C. was added 1 M LAH solution in THF (77.4 mL, 38.7 mmol, 2 eq). Reaction mixture was warmed to room temperature, then the mixture was stirred at 70° C. for 16 h under nitrogen atmosphere. TLC (5% MeOH in DCM) showed the reaction was incomplete. Reaction mixture was cooled to room temperature. The reaction was diluted with diethyl ether (30 mL), cooled the reaction mixture to 0° C., quenched with drop wise addition of 2.94 mL of water and 2.94 mL of 15% aqueous sodium hydroxide solution followed by addition of 8.8 mL of water (Fieser workup). The reaction mixture was stirred for 15 mins at room temperature. Then sodium sulphate was added to the reaction mixture, stirred for another 10 mins and the mixture was filtered through celite bed, washed with EtOAc (150 mL) and the filtrate was concentrated under reduced pressure to afford (2S)-2-amino-3-cyclopropylpropan-1-ol. LC-MS (ES) m/z=116.2 [M+H]+.

To a solution of (2S)-2-amino-3-cyclopropylpropan-1-ol (4.90 g, 42.5 mmol, 1.0 eq) in DCM (70 mL) under nitrogen atmosphere was added TEA (12 mL, 42.5 mmol, 2.0 eq) at 0° C. drop wise, stirred for 5 mins, then di-tert-butyl dicarbonate (11.7 mL, 42.5 mmol, 1.2 eq) was added. The reaction was stirred at room temperature for 16 h. Reaction was monitored by TLC (5% MeOH-DCM). After completion of the reaction, the reaction mixture was diluted with DCM (100 mL), washed with water (50 mL) and brine (25 mL), dried over Na₂SO₄, filtered and concentrated under reduced pressure to get the crude. The crude was purified by silica gel column chromatography using 2-3% MeOH in DCM as an eluent to afford tert-butyl N-[(2S)-1-cyclopropyl-3-hydroxypropan-2-yl]carbamate. 1H NMR (400 MHz, CDCl₃) δ ppm 0.08 (s, 2H), 0.48 (d, J=7.2 Hz, 2H), 0.67-0.69 (m, 1H), 1.44 (s, 9H), 1.62 (s, 2H), 2.47 (bs, 1H), 3.62-3.72 (m, 3H), 4.73 (bs, 1H).

To a solution of thionyl chloride (3.96 mL, 21.8 mmol, 2.5 eq) in DCM (50.0 mL) at −40° C. was added tert-butyl N-[(2S)-1-cyclopropyl-3-hydroxypropan-2-yl]carbamate (4.70 g, 21.8 mmol, 1.0 eq) in DCM (20 mL) and pyridine (9.14 mL, 21.8 mmol, 5.2 eq.). The mixture was stirred at −40° C. for 2 h under nitrogen atmosphere. TLC (20% EtOAc in hexane) showed the reaction was completed. The reaction was diluted with DCM:EtOAc (50 mL:50 mL) and the precipitate was filtered. The filtrate was washed with brine (50 mL). The organic layer was dried over anhydrous Na₂SO₄, filtered and concentrated under reduced pressure to get tert-butyl (4S)-4-(cyclopropylmethyl)-2-oxo-1,2λ⁴,3-oxathiazolidine-3-carboxylate. 1H NMR (400 MHz, CDCl3) δ ppm crude.

Tert-butyl (4S)-4-(cyclopropylmethyl)-2-oxo-1,2λ⁴,3-oxathiazolidine-3-carboxylate (4.80 g, 18.4 mmol, 1.0 eq) was dissolved in ACN (30 mL) and then ruthenium chloride (0.0533 g, 18.4 mmol, 0.014 eq) and sodium periodate (4.32 g, 18.4 mmol, 1.1 eq) was added at 0° C. and then water (30 mL) was added. The mixture was stirred at 0° C. for 15 mins and then at room temperature for 2 h. TLC (20% EtOAc in hexane) showed the reaction was completed. The reaction mixture was filtered through celite bed and the bed was washed with EtOAc (100 mL). The filtrate was washed with water (30 mL) and brine solution (20 mL). The organic layer was dried over anhydrous Na₂SO₄, filtered and concentrated under reduced pressure to get the crude. The crude was purified by flash chromatography using 10-15% EtOAc in hexane as an eluent to give tert-butyl (4S)-4-(cyclopropylmethyl)-2,2-dioxo-1,2λ⁶,3-oxathiazolidine-3-carboxylate. 1H NMR (400 MHz, CDCl3) δ ppm 0.15-0.16 (m, 2H), 0.48-0.59 (m, 2H), 0.64-0.66 (m, 1H), 1.48 (s, 9H), 1.67-1.73 (m, 1H), 1.79-1.87 (m, 1H), 4.37 (s, 1H), 4.46 (d, J=8.8 Hz, 1H), 4.65-4.69 (m, 1H).

To a solution of Copper(I) iodide (206 mg, 0.1 eq., 1.08 mmol) in diethyl ether (20 mL) was added bromo(3-methoxyphenyl)magnesium (21.6 mL, 2 eq., 21.6 mmol)_drop wise over a period of 10 min at −20° C. (salt & Ice mixture bath). The reaction mixture stirred for 30 min at −20° C. (salt & Ice mixture bath). After this time, a solution of tert-butyl N-[(2S)-1-cyclopropyl-3-(3-methoxyphenyl)propan-2-yl]carbamate (3.00 g, 9.82 mmol) in diethyl ether (10 mL) was added at −20° C. (salt & Ice mixture bath) drop wise over a period of 20 min to the reaction mass. The resulting mixture stirred for 3 h at −20° C. Reaction was monitored by TLC (10% EtOAc in n-hexane). After the completion of the reaction, the reaction mixture quenched with 10% aqueous citric acid solution (100 mL) at −20° C. (salt & Ice mixture bath). The mixture was allowed to warmed to RT and stirred for 10 min. The mixture was filtered through celite pad, washed with ethyl acetate thoroughly. The filtrate was washed with water (50 mL), brine (30 mL), dried over anhydrous sodium sulfate, filtered and concentrated to give the crude product. The crude was purified by silica gel column chromatography using 4-5% EtOAc in n-hexane as an eluent to afford tert-butyl N-[(2S)-1-cyclopropyl-3-(3-methoxyphenyl)propan-2-yl]carbamate. LC-MS (m/z)=250.2 ([M+H]+ after cleavage of boc group. 1H NMR (400 MHz, DMSO-d6) δ ppm −0.09 (s, 1H), 0.01 (s, 1H), 0.33-0.35 (m, 2H), 0.66 (s, 1H), 1.13-1.29 (s, 11H), 2.60-2.64 (m, 2H), 3.70 (s, 3H), 6.29-6.33 (m, 1H), 6.62 (d, J=8.8 Hz, 1H), 6.71 (s, 2H), 7.13 (t, J=7.2 Hz, 1H).

To a solution of tert-butyl N-[(2S)-1-cyclopropyl-3-(3-methoxyphenyl)propan-2-yl]carbamate (3.0 g, 9.82 mmol, 1.0 equiv.) in DCM (30.0 mL) under nitrogen atmosphere was added 4 M HCl in 1,4-Dioxane (8.0 mL) drop wise and the reaction mixture was stirred for 16 h at room temperature. Reaction was monitored by TLC (50% EtOAc in n-hexane). After completion of the reaction, the reaction mixture was concentrated under reduced pressure to get off white solid. The solid was then diluted with EtOAc (100 mL) and saturated sodium bicarbonate solution was added until pH ˜8. The mixture was stirred for 30 mins. The layers were separated and the aqueous layer was extracted with EtOAc (50 mL). Combined organic layers were dried over anhydrous sodium sulfate, filtered and concentrated under reduced pressure to afford (2S)-1-cyclopropyl-3-(3-methoxyphenyl)propan-2-amine. LC-MS (m/z)=206.2 ([M+H]⁺. 1H NMR (400 MHz, DMSO-d6) δ ppm 0.08-0.07 (m, 2H), 0.34-0.40 (m, 2H), 0.76 (s, 1H), 1.09-1.22 (m, 2H), 1.43-1.45 (m, 2H), 2.37-2.47 (m, 1H), 2.61-2.66 (m, 1H), 2.91-2.93 (m, 1H), 3.70 (s, 3H), 6.72 (s, 3H), 7.16 (t, J=7.8 Hz, 1H).

To a solution of 4-[(adamantan-1-yl)amino]benzoic acid (2.51 g, 9.25 mmol) and (2S)-1-cyclopropyl-3-(3-methoxyphenyl)propan-2-amine (1.90 g, 9.25 mmol) in (N,N-dimethylformamide (30.0 mL) was added N,N-dimethylpyridin-4-amine (2.83 g, 2.5 eq., 23.1 mmol), stirred for 5 min and then ({[3-(dimethylamino)propyl]imino}methylidene)(ethyl)amine hydrochloride (3.55 g, 2 eq., 18.5 mmol) was added at 0° C. This reaction mixture was stirred at room temperature for 16 h. Progress of the reaction was monitored by TLC (30% ethyl acetate in n-Hexane). After this time, the reaction mixture was diluted with EtOAc and saturated sodium bicarbonate solution. Organic layer was separated, washed with water, brine solution, dried over anhydrous sodium sulfate, filtered and concentrated under reduced pressure to get the crude product. Obtained crude product was purified by flash chromatography on silica gel. Desired product was eluted at 30% ethyl acetate in n-Hexane. Fractions containing product were combined and concentrated under reduced pressure to get 4-[(adamantan-1-yl)amino]-N-[(2S)-1-(3-methoxyphenyl)hexan-2-yl]benzamide. LC-MS (ES) (m/z)=458.6 [M+H]⁺. ¹H NMR (400 MHz, DMSO) δ ppm 0.039-0.051 (m, 2H), 0.34 (d, J=7.6 Hz, 2H), 0.71 (bs, 1H), 1.29-1.47 (m, 2H), 1.64 (s, 6H), 1.81 (s, 6H), 2.05 (s, 3H), 2.77-2.79 (m, 2H), 3.67 (s, 3H), 4.16-4.19 (m, 1H), 5.52 (s, 1H), 6.69 (d, J=8.4 Hz, 3H), 6.76-6.78 (m, 2H), 7.13 (t, J=8.0 Hz, 1H), 7.51 (d, J=8.8 Hz, 2H), 7.70 (d, J=8.4 Hz, 1H).

To stirred solution of 4-[(adamantan-1-yl)amino]-N-[(2S)-1-cyclopropyl-3-(3-methoxyphenyl)propan-2-yl]benzamide (1.25 g, 2.73 mmol) and 2-chloropyridine (1.55 mL, 6 eq., 16.4 mmol) in DCM (30 mL) was added trifluoromethanesulfonic anhydride (15 mL, 89.2 mmol, 3.0 equiv) via syringe slowly drop wise at −78° C. After 5 min, the reaction mixture was placed in an ice-water bath and warmed to 0° C. After 5 min, the resulting solution was allowed to stir at room temperature for 1.5 h. Reaction mixture was quenched with aqueous sodium hydroxide solution (50 mL, 1N) to neutralize the trifluoromethanesulfonate salts. Dichloromethane (50 mL) was added to dilute the mixture and the layers were separated. The aqueous layer was extracted with DCM (30 mL). The combined organic layer was washed with brine (25 mL), dried over anhydrous sodium sulfate and filtered. Organic layer was filtered and concentrated under reduced pressure to give the crude product. LCMS (ES)m/z: 440.6 [M+H]⁺. ¹H NMR (400 MHz, DMSO) δ ppm: 0.06-0.07 (m, 1H), 0.43 (d, J=7.2 Hz, 2H), 0.90 (bs, 1H), 1.32-1.49 (m, 2H), 1.40-1.49 (m, 1H) 1.64 (s, 6H), 1.80 (s, 6H), 2.06 (s, 3H), 2.55-2.58 (m, 1H), 2.86-2.89 (m, 1H), 3.45 (bs, 1H), 3.8 (s, 3H), 5.57 (ds, 1H), 6.77-6.85 (m, 3H), 6.93 (s, 1H), 7.28 (d, J=8.4, 3H).

To a solution of N-{4-[(3S)-3-(cyclopropylmethyl)-6-methoxy-3,4-dihydroisoquinolin-1-yl]phenyl}adamantan-1-amine (1.00 g, 2.27 mmol) in methanol (5.00 mL) was added sodium boranuide (258 mg, 3 eq., 6.81 mmol) at 0° C. portion wise. The suspension was stirred at RT for 2 h. After this time, the reaction mixture was concentrated and obtained crude was diluted with EtOAc and water. Organic layer was separated, washed with brine solution, dried over anhydrous sodium sulfate, filtered and concentrated under reduced pressure to get the crude product. The crude was purified by flash chromatography using 35-45% EtOAc in hexane as an eluent to give (1S,3R,5S)—N-(4-((1R,3S)-3-(cyclopropylmethyl)-6-methoxy-1,2,3,4-tetrahydroisoquinolin-1-yl)phenyl)adamantan-1-amine (0.4 g, 39.82%) and (1S,3R,5S)—N-(4-((1S,3S)-3-(cyclopropylmethyl)-6-methoxyl,2,3,4 tetrahydroisoquinolin-1-yl)phenyl)adamantan-1-amine. LC-MS (ES) (m/z)=442.6 [M+H]+ (for cis isomer) and LC-MS (ES) (m/z)=442.6 [M+H]⁺ (for trans isomer). ¹H NMR (400 MHz, DMSO) δ ppm; 0.05 (bs, 2H), 0.34 (m, 2H), 0.66 (bs, 1H), 0.81-0.92 (m, 1H), 1.20 (s, 3H), 1.32-1.43 (m, 2H), 1.59 (s, 6H), 1.80 (s, 6H), 2.00 (s, 3H), 2.85-2.89 (m, 1H), 4.97 (s, 1H) 5.52 (s, 1H), 2.97 (bs, 1H), 6.62-6.72 (m, 4H), 6.78-6.87 (m, 3H).

### Intermediate Procedure B-5: Synthetic Scheme for Compound 4-(((3R,5R,7R)-adamantan-1-yl)amino)-N—((S)-1-(3-hydroxyphenyl)hexan-2-yl)benzamide

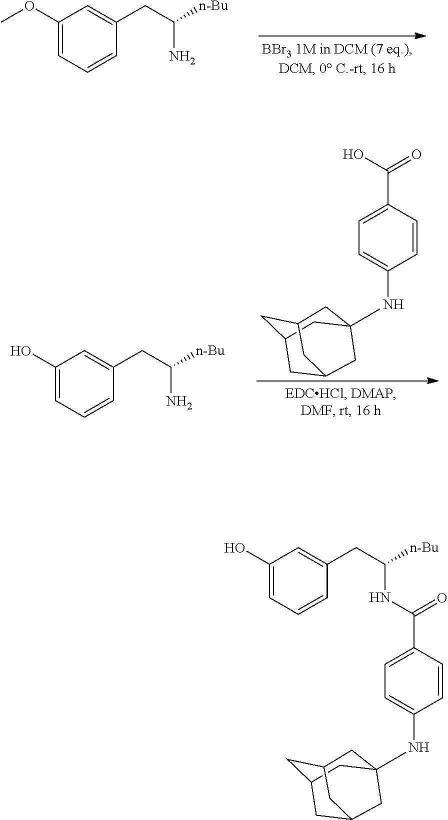

To stirred solution of (S)-1-(3-methoxyphenyl)hexan-2-amine (2.0 g, 9.65 mmol, 1 equiv.) in DCM (20 mL) was added Boron tribromide solution 1.0 M in methylene chloride (67.5 mL, 67.50 mmol, 7.0 equiv.) at 0° C. This reaction mass was stirred at RT for 16 h. Progress of the reaction was monitored by LCMS. After this time, reaction mixture was concentrated under reduced pressure and obtained crude was quenched with saturated aqueous NaHCO₃ solution (20 mL) to neutralize the trace of BBr₃. Dichloromethane (75 mL) was added to dilute the mixture and the layers were separated. The aqueous layer was extracted with DCM (30 mL). The combined organic layer was washed with brine (25 mL), was dried over anhydrous sodium sulfate, and was filtered. The volatiles were removed under reduced pressure to give the product. LC-MS (m/z)=194.2 [M+H]⁺. ¹H NMR (400 MHz, DMSO-d₆) δ ppm 0.82-0.88 (m, 3H), 1.06-1.34 (m, 6H), 2.35-2.38 (m, 1H), 2.48-2.59 (m, 1H), 2.81-2.82 (m, 1H), 6.56-6.58 (m, 3H), 7.04 (t, J=8.0 Hz, 1H), 9.20 (bs, 1H). NH₂ protons were not observed (might be exchanged with DMSO-d₆ solvent moisture).

To a solution of 4-(((3s,5s,7s)-adamantan-1-yl)amino)benzoic acid (2.06 g, 7.61 mmol, 1.05 equiv.) and (S)-3-(2-aminohexyl)phenol (1.4 g, 7.24 mmol, 1.0 equiv.) in DMF (15 mL) was added DMAP (2.21 g, 18.10 mmol, 2.5 equiv.), stirred for 5 min and then EDC·HCl (2.78 g, 14.50 mmol, 2.0 equiv.) was added at 0° C. This reaction mixture was stirred at room temperature for 16 h. Progress of the reaction was monitored by TLC (40% ethyl acetate in n-Hexane). After this time, the reaction mixture was diluted with EtOAc (75 mL) and saturated sodium bicarbonate solution (15 mL). Organic layer was separated, washed with water (5×15 mL), 1N HCl solution (2×15 mL), saturated sodium bicarbonate solution (15 mL), brine solution (15 mL), dried over anhydrous sodium sulfate, filtered and concentrated under reduced pressure to get the crude. The resulting residue was purified by flash column chromatography (eluent:EtOAc in n-Hexanes) on silica gel to give 4-(((3R,5R,7R)-adamantan-1-yl)amino)-N—((S)-1-(3-hydroxyphenyl)hexan-2-yl)benzamide. LC-MS (ES) (m/z)=447.3 [M+H]⁺. 1H NMR (400 MHz, DMSO-d₆) δ ppm 0.81-0.83 (m, 3H), 1.05-1.17 (m, 5H), 1.38-1.47 (m, 1H), 1.64 (m, 6H), 1.88-1.95 (m, 6H), 2.06 (m, 3H), 2.72-2.84 (m, 2H), 4.00-4.09 (m, 1H), 5.53 (s, 1H), 6.22 (s, 1H), 6.68 (d, J=8.4 Hz, 1H), 6.80 (d, J=8.4 Hz, 1H), 6.94-7.09 (m, 2H), 7.27 (d, J=7.8 Hz, 1H), 7.49 (d, J=8.4 Hz, 1H), 7.69-7.748 (m, 2H). Adamantine attached NH protons was not observed (might be exchanged with DMSO-d₆ solvent moisture)

### Intermediate Procedure B-6: Synthetic Scheme for Compound (3S,5S,7S)—N-(4-((1R,3S)-3-butyl-6-fluoro-1,2,3,4-tetrahydroisoquinolin-1-yl)phenyl)adamantan-1-amine

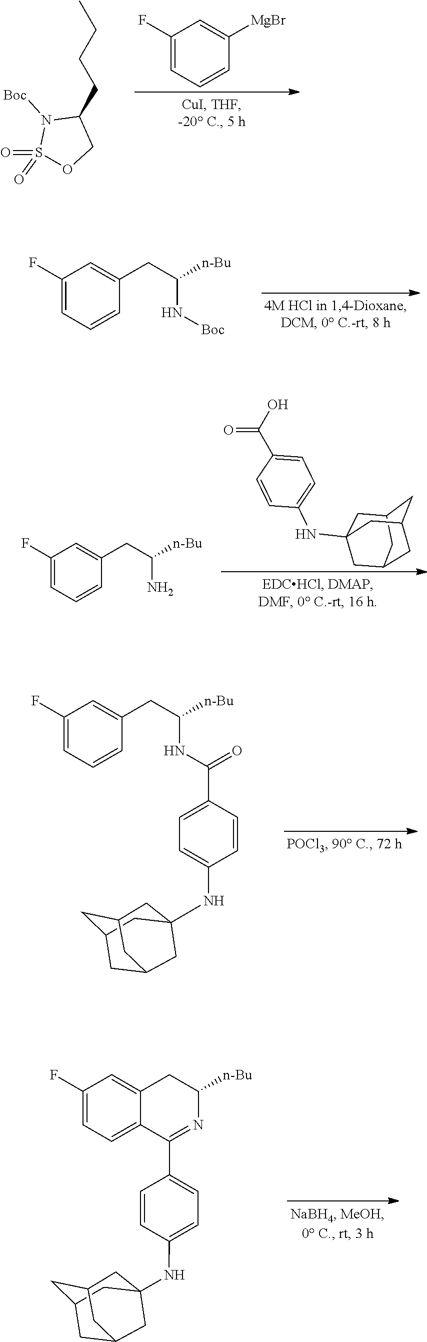

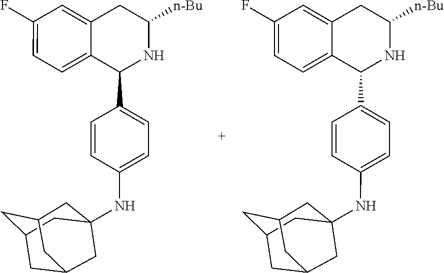

To a stirred solution of CuI (0.818 g, 4.30 mmol, 0.2 equiv.) in tetrahydrofuran (60 mL) was added 1 M solution of (3-fluorophenyl)magnesium bromide (64.4 mL, 64.4 mmol, 3.0 equiv.) in tetrahydrofuran drop wise over a period of 15 min at −20° C., stirred for 30 min. A solution of tert-butyl (S)-4-butyl-1,2,3-oxathiazolidine-3-carboxylate 2,2-dioxide (6.0 g, 21.5 mmol, 1.0 equiv.) in tetrahydrofuran (60 mL) was added drop wise at −20° C. and the reaction mixture was allowed to stir for 4 h at −20° C. The reaction was monitored by TLC. After completion, the reaction mixture was quenched with 10% aqueous citric acid solution at −20° C., allowed to warm to room temperature and stirred for 15 min. The mixture was filtered through celite pad and washed with ethyl acetate. The ethyl acetate layer was separated from the filtrate, washed with water and brine solution, dried over sodium sulphate, filtered and concentrated to obtained crude product, which was purified by flash column chromatography using gradient of 0-10% ethyl acetate in hexane over 230-400 mesh silica gel. The desired fractions were concentrated to afford tert-butyl (S)-(1-(3-fluorophenyl)hexan-2-yl)carbamate. LC-MS (m/z)=240.2 [M+H]⁺−56. 1H NMR (400 MHz, CDCl3) δ ppm 0.87-0.88 (m, 3H), 1.31-1.65 (m, 15H), 1.74-1.75 (m, 2H), 3.78 (bs, 1H), 4.28 (s, 1H), 6.56-6.63 (m, 1H), 6.87-6.95 (m, 2H), 7.13-7.26 (m, 1H).

To a stirred solution of tert-butyl (S)-(1-(3-fluorophenyl)hexan-2-yl)carbamate (600 mg, 2.03 mmol, 1.0 equiv.) in DCM (1.5 mL) was added 4 M HCl in 1,4-Dioxane (5.08 mL, 20.3 mmol, 10.0 equiv.) at 0° C. and the reaction mixture was stirred at room temperature for 8 h. The progress of reaction was monitored by TLC. After completion, the reaction mixture was concentrated under reduced pressure and the residue part was basified with aqueous sodium bicarbonate solution, product was extracted with ethyl acetate. The organic layer was washed with brine, dried over sodium sulphate, filtered and concentrated to afford (S)-1-(3-fluorophenyl)hexan-2-amine. LC-MS (ES) (m/z)=196.1 [M+H]+.

To a solution of 4-(((3s,5s,7s)-adamantan-1-yl)amino)benzoic acid (0.385 g, 1.42 mmol, 1.05 equiv.) and (S)-1-(3-fluorophenyl)hexan-2-amine (264 mg, 1.35 mmol, 1.0 equiv.) in DMF (5.0 mL) was added DMAP (413 mg, 3.38 mmol, 2.5 equiv.), stirred for 5 min and then EDC·HCl (518 mg, 2.70 mmol, 2.0 equiv.) was added at 0° C. This reaction mixture was stirred at room temperature for 16 h. Progress of the reaction was monitored by TLC (30% ethyl acetate in n-Hexane). After this time, the reaction mixture was diluted with EtOAc (25 mL) and saturated sodium bicarbonate solution (5 mL). Organic layer was separated, washed with water (5×5 mL), 1N HCl solution (2×5 mL), saturated sodium bicarbonate solution (5 mL), brine solution (4 mL), dried over anhydrous sodium sulfate, filtered and concentrated under reduced pressure to get the desired product (420 mg, 69%) as brown gum. The resulting residue was used in the next reaction without any further purification. LC-MS (ES) (m/z)=449.3 [M+H]⁺.

To stirred solution of 4-(((3R,5R,7R)-adamantan-1-yl)amino)-N—((S)-1-(3-fluorophenyl)hexan-2-yl)benzamide (330 mg, 0.736 mmol, 1 equiv.) in POCl₃ (2.75 mL, 29.40 mmol, 40 equiv.) was heated at 90° C. for 72 h. Progress of the reaction was monitored by TLC (40% EA in n-Hexane). After this time, reaction mixture was concentrated under reduced pressure and obtained crude was quenched with aqueous sodium hydroxide solution (10 mL, 1N aqueous NaOH solution) to neutralize the trace of POCl₃. Dichloromethane (35 mL) was added to dilute the mixture and the layers were separated. The aqueous layer was extracted with DCM (10 mL). The combined organic layer was washed with brine (5.0 mL), was dried over anhydrous sodium sulfate, and was filtered. The volatiles were removed under reduced pressure to give the crude product (250 mg, crude). The resulting residue was used in the next reaction without any further purification. LC-MS (ES) (m/z)=431.3 [M+H]⁺.

To a solution of (3R,5R,7R)—N-(4-((S)-3-butyl-6-fluoro-3,4-dihydroisoquinolin-1-yl)phenyl)adamantan-1-amine (250 mg, 0.58 mmol, 1.0 equiv.) in methanol (4.0 mL) was added sodium borohydride (62.4 mg, 1.74 mmol, 3.0 equiv.) at −78° C. portion wise. The suspension was stirred at room temperature for 3 h. After this time, the reaction mixture was concentrated and obtained crude was diluted with EtOAc (30 mL) and water (5 mL). Organic layer was separated, washed with brine solution (5 mL), dried over anhydrous sodium sulfate, filtered and concentrated under reduced pressure to get the crude product (260 mg).

Obtained crude product was purified by preparative TLC using 35% EtOAc in n-Hexane as mobile phase to get (3R,5R,7R)—N-(4-((1S,3S)-3-butyl-6-fluoro-1,2,3,4-tetrahydroisoquinolin-1-yl)phenyl)adamantan-1-amine (25 mg, 10%) as gum (1.3 trans isomer) and (3S,5S,7S)—N-(4-((1R,3S)-3-butyl-6-fluoro-1,2,3,4-tetrahydroisoquinolin-1-yl)phenyl)adamantan-1-amine (this was eluted along with same Rf impurities) (1.3 cis isomer).

150 mg of the crude (1,3-cis isomer which was eluted along with same Rf impurities) was repurified by preparative TLC using 25% EtOAc in n-Hexane as mobile phase to get N-{4-[(1R,3S)-3-butyl-6-fluoro-1,2,3,4-tetrahydroisoquinolin-1-yl]phenyl}adamantan-1-amine. Analytical data: for Trans Isomer: LC-MS (ES) (m/z)=433.3 [M+H]⁺. ¹H NMR (400 MHz, CDCl₃): δ ppm 0.85 (t, J=6.6 Hz, 3H), 1.23-1.29 (m, 4H), 1.43-1.47 (m, 2H), 1.67 (s, 6H), 1.86 (s, 6H), 2.09 (s, 3H), 2.59-2.65 (m, 1H), 2.75-2.87 (m, 1H), 3.00 (bs, 1H), 5.16 (s, 1H), 6.69 (d, J=8.4 Hz, 2H), 6.77-6.92 (m, 5H). Analytical data: FRIN01-JBL-91198-P-01 for Cis Isomer. LC-MS (ES) (m/z)=433.3 [M+H]⁺. ¹H NMR (400 MHz, CDCl₃): δ ppm 0.91 (t, J=6.8 Hz, 3H), 1.34-1.39 (m, 4H), 1.52-1.56 (m, 2H), 1.63 (s, 6H), 1.87 (s, 6H), 2.10 (s, 3H), 2.56-2.81 (m, 1H), 3.01 (t, J=6.0 Hz, 2H), 4.92 (s, 1H), 6.67-6.79 (m, 5H), 7.07 (d, J=8.0 Hz, 2H).

### Intermediate Procedure B-10: Synthesis of 4-((1R,3S)-3-(cyclopropylmethyl)-6-methoxy-1,2,3,4-tetrahydroisoquinolin-1-yl)benzonitrile and 4-((1S,3S)-3-(cyclopropylmethyl)-6-methoxy-1,2,3,4-tetrahydroisoquinolin-1-yl)benzonitrile

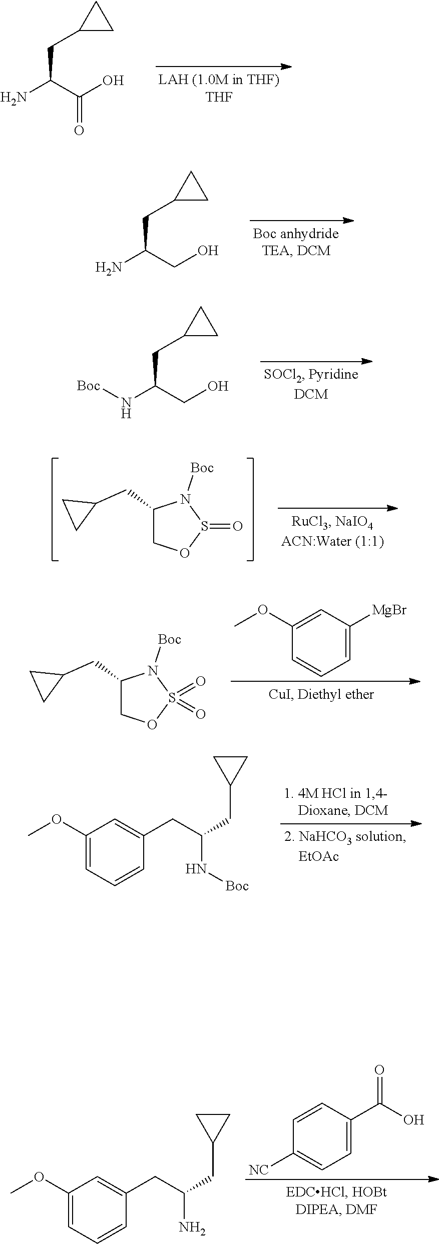

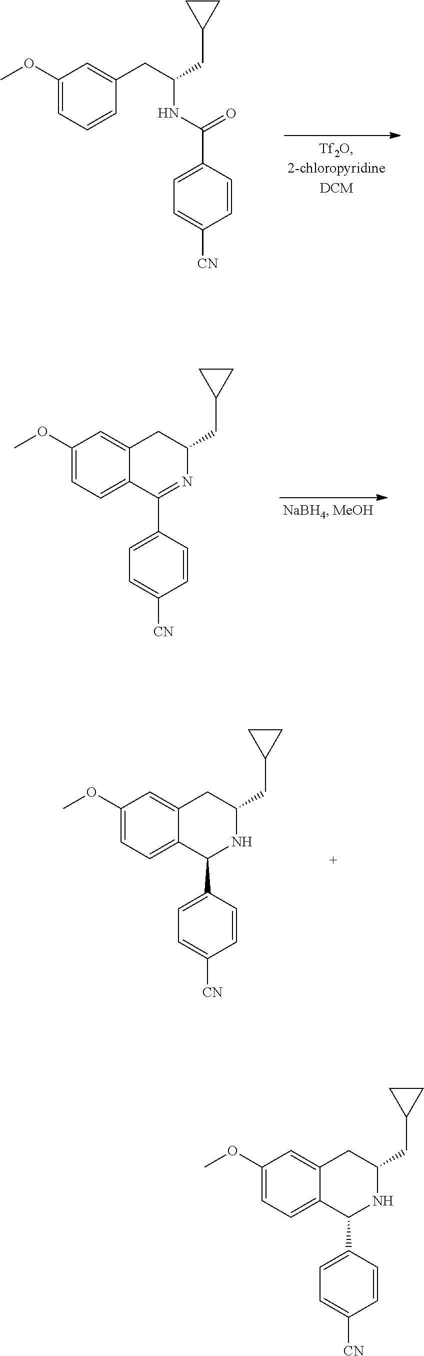 [SMILES: COc1cccc(C[C@H](CC2CC2)NC(=O)c2ccc(C#N)cc2)c1 ; COc1ccc2c(c1)C[C@H](CC1CC1)N=C2c1ccc(C#N)cc1 ; COc1ccc2c(c1)C[C@H](CC1CC1)N[C@H]2c1ccc(C#N)cc1 ; "+" ; COc1ccc2c(c1)C[C@H](CC1CC1)N[C@@H]2c1ccc(C#N)cc1]

To a solution of (2S)-2-amino-3-cyclopropylpropanoic acid (7.0 g, 54.2 mmol, 1 eq) in THF (100.0 mL) at 0° C. was added 1 M LAH solution in THF (108 mL, 108 mmol, 2 eq). Reaction mixture was warmed to room temperature, then the mixture was stirred at 70° C. for 16 h under nitrogen atmosphere. TLC (5% MeOH in DCM) showed the reaction was incomplete. Reaction mixture was cooled to room temperature. The reaction was diluted with diethyl ether (30 mL), cooled the reaction mixture to 0° C., quenched with drop wise addition of 4.11 mL of water and 4.11 mL of 15% aqueous sodium hydroxide solution followed by addition of 12 mL of water (Fieser workup). The reaction mixture was stirred for 15 mins at room temperature. Then sodium sulphate was added to the reaction mixture, stirred for another 10 mins and the mixture was filtered through celite bed, washed with EtOAc (150 mL) and the filtrate was concentrated under reduced pressure to afford (2S)-2-amino-3-cyclopropylpropan-1-ol. LC-MS (ES) m/z=116.2 [M+H]+.

To a solution of (2S)-2-amino-3-cyclopropylpropan-1-ol (6.64 g, 57.7 mmol, 1.0 eq) in DCM (70 mL) under nitrogen atmosphere was added TEA (16.2 mL, 115 mmol, 2.0 eq) at 0° C. drop wise, stirred for 5 mins, then di-tert-butyl dicarbonate (15.9 mL, 69.2 mmol, 1.2 eq) was added. The reaction was stirred at room temperature for 16 h. Reaction was monitored by TLC (5% MeOH-DCM). After completion of the reaction, the reaction mixture was diluted with DCM (100 mL), washed with water (50 mL) and brine (25 mL), dried over Na₂SO₄, filtered and concentrated under reduced pressure to get the crude. The crude was purified by silica gel column chromatography using 2-3% MeOH in DCM as an eluent to afford tert-butyl N-[(2S)-1-cyclopropyl-3-hydroxypropan-2-yl]carbamate. ¹H NMR (400 MHz, CDCl3) δ ppm 0.08 (s, 2H), 0.48 (d, J=7.2 Hz, 2H), 0.67-0.69 (m, 1H), 1.44 (s, 9H), 1.62 (s, 2H), 2.47 (bs, 1H), 3.62-3.72 (m, 3H), 4.73 (bs, 1H).

To a solution of thionyl chloride (5.73 mL, 79.0 mmol, 2.5 eq) in DCM (50.0 mL) at −40° C. was added tert-butyl N-[(2S)-1-cyclopropyl-3-hydroxypropan-2-yl]carbamate (6.8 g, 31.6 mmol, 1.0 eq) in DCM (20.0 mL) and pyridine (13.2 mL, 164 mmol, 5.2 eq.). The mixture was stirred at −40° C. for 2 h under nitrogen atmosphere. TLC (20% EtOAc in hexane) showed the reaction was completed. The reaction was diluted with DCM:EtOAc (50 mL:50 mL) and the precipitate was filtered. The filtrate was washed with brine (50 mL). The organic layer was dried over anhydrous Na₂SO₄, filtered and concentrated under reduced pressure to get tert-butyl (4S)-4-(cyclopropylmethyl)-2-oxo-1,2λ⁴,3-oxathiazolidine-3-carboxylate.

Tert-butyl (4S)-4-(cyclopropylmethyl)-2-oxo-1,2λ⁴,3-oxathiazolidine-3-carboxylate (8.2 g, 31.4 mmol, 1.0 eq) was dissolved in ACN (30 mL) and then ruthenium chloride (0.032 g, 0.157 mmol, 0.005 eq) and sodium periodate (7.38 g, 34.5 mmol, 1.1 eq) was added at 0° C. and then water (30 mL) was added. The mixture was stirred at 0° C. for 15 mins and then at room temperature for 2 h. TLC (20% EtOAc in hexane) showed the reaction was completed. The reaction mixture was filtered through celite bed and the bed was washed with EtOAc (100 mL). The filtrate was washed with water (30 mL) and brine solution (20 mL). The organic layer was dried over anhydrous Na₂SO₄, filtered and concentrated under reduced pressure to get the crude. The crude was purified by flash chromatography using 10-15% EtOAc in hexane as an eluent to give tert-butyl (4S)-4-(cyclopropylmethyl)-2,2-dioxo-1,2λ⁶,3-oxathiazolidine-3-carboxylate. 1H NMR (400 MHz, CDCl3) δ ppm 0.15-0.16 (m, 2H), 0.48-0.59 (m, 2H), 0.64-0.66 (m, 1H), 1.48 (s, 9H), 1.67-1.73 (m, 1H), 1.79-1.87 (m, 1H), 4.37 (s, 1H), 4.46 (d, J=8.8 Hz, 1H), 4.65-4.69 (m, 1H).

To a solution of copper iodide (0.231 g, 1.21 mmol, 0.1 eq) in diethyl ether (20 mL) was added (3-methoxyphenyl)magnesium bromide (1 M in THF) (24.2 mL, 24.2 mmol, 2.0 eq) drop wise over a period of 10 min at −20° C. (salt & Ice mixture bath). The reaction mixture stirred for 30 min at −20° C. (salt & Ice mixture bath). After this time, a solution of tert-butyl (4S)-4-(cyclopropylmethyl)-2,2-dioxo-1,2λ⁶,3-oxathiazolidine-3-carboxylate (3.36 g, 12.1 mmol, 1.0 eq) in diethyl ether (10 mL) was added at −20° C. (salt & Ice mixture bath) drop wise over a period of 20 min to the reaction mass. The resulting mixture stirred for 3 h at −20° C. Reaction was monitored by TLC (10% EtOAc in n-hexane). After completion of the reaction, the reaction mixture was quenched with 10% aqueous citric acid solution (100 mL) at −20° C. (salt & ice mixture bath). The mixture was allowed to warm to room temperature and stirred for 10 min. The mixture was filtered through celite pad, washed with ethyl acetate thoroughly. The filtrate was washed with water (50 mL), brine (30 mL), dried over anhydrous sodium sulfate, filtered and concentrated to give the crude product. The crude was purified by silica gel column chromatography using 4-5% EtOAc in n-hexane as an eluent to afford tert-butyl N-[(2S)-1-cyclopropyl-3-(3-methoxyphenyl)propan-2-yl]carbamate. LC-MS (ES) (m/z)=250.2 [M+H]+ after cleavage of boc group. 1H NMR (400 MHz, DMSO-d6) δ ppm −0.09 (s, 1H), 0.01 (s, 1H), 0.33-0.35 (m, 2H), 0.66 (s, 1H), 1.13-1.29 (s, 11H), 2.60-2.64 (m, 2H), 3.70 (s, 3H), 6.29-6.33 (m, 1H), 6.62 (d, J=8.8 Hz, 1H), 6.71 (s, 2H), 7.13 (t, J=7.2 Hz, 1H).

To a solution of tert-butyl N-[(2S)-1-cyclopropyl-3-(3-methoxyphenyl)propan-2-yl]carbamate (3.0 g, 9.82 mmol, 1.0 eq) in DCM (30.0 mL) under nitrogen atmosphere was added 4 M HCl in 1,4-Dioxane (8.0 mL) drop wise and the reaction mixture was stirred for 16 h at room temperature. Reaction was monitored by TLC (50% EtOAc in n-hexane). After completion of the reaction, the reaction mixture was concentrated under reduced pressure to get off white solid. The solid was then diluted with EtOAc (100 mL) and saturated sodium bicarbonate solution was added until pH ˜8. The mixture was stirred for 30 mins. The layers were separated and the aqueous layer was extracted with EtOAc (50 mL). Combined organic layers were dried over anhydrous sodium sulphate, filtered and concentrated under reduced pressure to afford (2S)-1-cyclopropyl-3-(3-methoxyphenyl)propan-2-amine. LC-MS (ES) (m/z)=206.2 ([M+H]⁺. 1H NMR (400 MHz, DMSO-d6) δ ppm −0.08-−0.07 (m, 2H), 0.34-0.40 (m, 2H), 0.76 (s, 1H), 1.09-1.22 (m, 2H), 1.43-1.45 (m, 2H), 2.37-2.47 (m, 1H), 2.61-2.66 (m, 1H), 2.91-2.93 (m, 1H), 3.70 (s, 3H), 6.72 (s, 3H), 7.16 (t, J=7.8 Hz, 1H).

To a stirred solution of 4-cyanobenzoic acid (1.81 g, 12.3 mmol, 1.3 eq) in DMF (20 mL) under nitrogen atmosphere was added DIPEA (5.23 mL, 28.3 mmol, 3 eq), stirred for 5 minutes, then EDC·HCl (2.72 g, 14.2 mmol, 1.5 eq) followed by (2S)-1-cyclopropyl-3-(3-methoxyphenyl)propan-2-amine (1.94 g, 9.45 mmol, 1 eq) was added at 0° C., stirred for another 5 minutes and then HOBt (1.92 g, 14.2 mmol, 1.5 eq) was added to the reaction mixture at 0° C. and then reaction mixture was stirred at room temperature for 16 h. Reaction was monitored by TLC (30% EtOAc in n-hexane). After this time, the reaction mixture was diluted with ice-water and stirred for 5 minutes, and then extracted with EtOAc (2×100 mL). Combined organic layer was washed with saturated sodium bicarbonate solution (30 mL) and water (50 mL). Organic layer was dried over anhydrous sodium sulphate, filtered and concentrated under reduced pressure to get the crude. The crude was purified by silica gel column chromatography using 15-20% EtOAc in n-hexane as an eluent to afford 4-cyano-N-[(2S)-1-cyclopropyl-3-(3-methoxyphenyl)propan-2-yl]benzamide. LC-MS (ES) m/z: 335.3 [M+H]+. 1H NMR (400 MHz, DMSO-d6) δ ppm 0.04-0.05 (m, 2H), 0.34-0.36 (m, 2H), 1.21 (bs, 1H), 1.38-1.48 (m, 2H), 2.74-2.84 (m, 2H), 3.65 (s, 3H), 4.22-4.24 (m, 1H), 6.69 (d, J=8.0 Hz, 1H), 6.68-6.77 (m, 2H), 7.13 (t, J=7.8 Hz, 1H), 7.90 (q, J=7.2 Hz, 4H), 8.45 (d, J=8.0 Hz, 1H).

To a stirred solution of 4-cyano-N-[(2S)-1-cyclopropyl-3-(3-methoxyphenyl)propan-2-yl]benzamide (2.7 g, 8.07 mmol, 1.0 eq) in DCM (25.0 mL) under nitrogen atmosphere was added 2-chloropyridine (2.29 mL, 24.2 mmol, 3 eq) at room temperature and stirred for 5 minutes, cooled the reaction mixture to −78° C. and then triflic anhydride (4.07 mL, 24.2 mmol, 3.0 eq) was added, stirred for 5 mins, warmed to 0° C. and stirred at 0° C. for 5 mins and then reaction mixture was stirred at room temperature for 1 h. The reaction was monitored by TLC (20% EtOAc in n-hexane) and LC-MS. After this time, the reaction mixture was quenched with 1N NaOH (pH˜14), stirred for 5 mins and was extracted with EtOAc (150 mL). Organic layer was dried over anhydrous sodium sulphate, filtered and concentrated under reduced pressure to get the crude. The crude was purified by silica gel column chromatography using 10-15% EtOAc in n-hexane as an eluent to afford 4-[(3S)-3-(cyclopropylmethyl)-6-methoxy-3,4-dihydroisoquinolin-1-yl]benzonitrile. LC-MS (ES) m/z: 317.2 [M+H]+.

To a stirred solution of 4-[(3S)-3-(cyclopropylmethyl)-6-methoxy-3,4-dihydroisoquinolin-1-yl]benzonitrile (2.59 g, 5.73 mmol, 1.0 eq) in MeOH (30.0 mL) under nitrogen atmosphere was added sodium borohydride (0.65 g, 17.2 mmol, 3 eq) at 0° C. and the reaction mixture was stirred at room temperature for 2 h. The reaction was monitored by TLC (40% EtOAc in n-hexane) and LC-MS. After this time, the reaction mixture was concentrated under reduced pressure and the crude was dissolved in EtOAc (150 mL) and washed with water (30 mL). Organic layer was dried over anhydrous sodium sulfate, filtered and concentrated under reduced pressure to get the crude. The crude was purified by silica gel column chromatography using 30-35% EtOAc in n-hexane as an eluent to afford 4-[(1S,3S)-3-(cyclopropylmethyl)-6-methoxy-1,2,3,4-tetrahydroisoquinolin-1-yl]benzonitrile. LC-MS (ES) m/z: 319.2 [M+H]⁺. ¹HNMR (400 MHz, CDCl₃) δ ppm −0.023-0.06 (m, 2H), 0.37-0.42 (m, 2H), 0.60 (bs, 1H), 1.13-1.38 (m, 3H), 2.58-2.64 (m, 1H), 2.85-2.94 (m, 2H), 3.80 (s, 3H), 5.21 (s, 1H), 6.68-6.70 (m, 2H), 6.76-6.78 (m, 1H), 7.25-7.29 (m, 2H), 7.57 (d, J=7.6 Hz, 2H) while purification Cis isomer also isolated separately.

### Procedure 17: Synthesis of Compound B-32

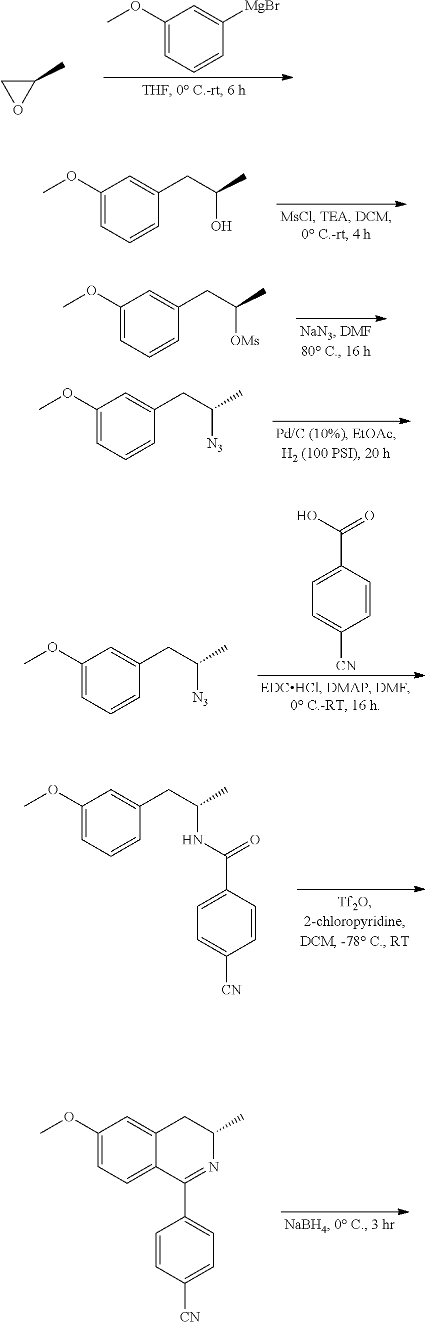

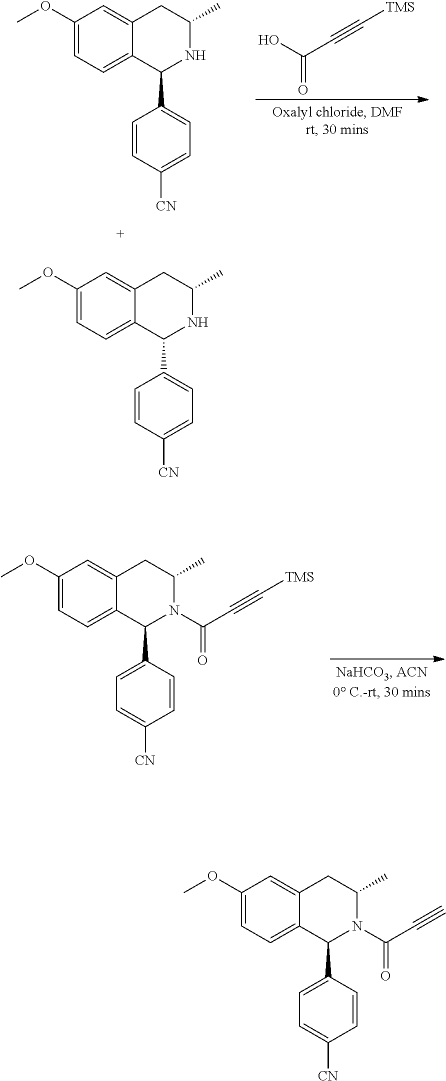

To a solution of (R)-2-methyloxirane (10 g, 172 mmol, 1.0 equiv) in THF (150 mL) was added (3-methoxyphenyl)magnesium bromide (207 mL, 207 mmol, 1.2 equiv) drop wise at 0° C. The reaction mixture was stirred at room temperature for 6 hours. After this time, the reaction mixture was quenched with aqueous ammonium chloride solution (100 mL) and product was extracted in to ethyl acetate (100 mL). The organic layer was washed with water (2×200 mL), brine (100 mL), dried over anhydrous NaSO4, filtered and concentrated under reduced pressure to get the crude product. The mixture was purified by silica gel flash column chromatography (n-hexane/EtOAc=8-10%) to give (R)-1-(3-methoxyphenyl)propan-2-ol. ¹H NMR (400 MHz, DMSO-d6) δ ppm 1.00 (d, J=6.4 Hz, 3H), 2.48-2.52 (m, 1H), 2.62-2.67 (m, 1H), 3.70 (s, 3H), 3.76-3.82 (m, 1H), 4.51 (d, J=4.8 Hz, 1H), 6.71-6.73 (m, 3H), 7.14 (t, J=7.8 Hz, 1H).

To a solution of (2R)-1-(3-methoxyphenyl)propan-2-ol (12.5 g, 75.2 mmol, 1.0 equiv) in DCM (150 mL) was added triethyl amine (31.7 ml, 226 mmol, 3.0 equiv) at 0° C., followed by mesyl chloride (9.23 mL, 113 mmol, 1.5 equiv). The mixture was stirred at 0° C. for 1.0 h under N₂ atmosphere. TLC (30% EtOAc in n-Hexane) showed the reaction was completed. Then the reaction was diluted with saturated aqueous solution of NaHCO₃ (100 mL) and was extracted with DCM (150 mL). The organic layer was dried over anhydrous Na₂SO₄, filtered and concentrated under reduced pressure to give (2R)-1-(3-methoxyphenyl)propan-2-yl methanesulfonate. ¹H NMR (400 MHz, CDCl₃) δ ppm 1.47 (d, J=6.0 Hz, 3H), 2.56 (s, 3H), 2.84-2.89 (m, 1H), 2.93-2.99 (m, 1H), 3.79 (s, 3H), 4.87-4.92 (m, 1H), 6.77-6.82 (m, 3H), 7.21-7.23 (m, 1H).

To a solution of (2R)-1-(3-methoxyphenyl)propan-2-yl methanesulfonate (15.0 g, 61.4 mmol, 1.0 equiv) in DMF (150 mL) was added sodium azide (4.79 g, 73.7 mmol, 1.2 equiv) at room temperature. The mixture was stirred at 80° C. for 16 h. TLC (5% EtOAc in n-Hexane) showed the reaction was completed. Then the reaction was diluted with water (150 mL) and EtOAc (200 mL). The organic layer was separated, washed with water (2×100 mL), brine (100 mL), dried over anhydrous NaSO₄, filtered and concentrated under reduced pressure to get the crude product. Obtained crude product was purified by silica gel flash column chromatography (n-hexane/EtOAc 3-5%) to give 1-[(2S)-2-azidopropyl]-3-methoxybenzene. ¹H NMR (400 MHz, CDCl₃) δ ppm 1.26 (d, J=6.0 Hz, 3H), 2.67-2.71 (m, 1H), 2.78-2.83 (m, 1H), 3.67-3.69 (m, 1H), 3.80 (s, 3H), 6.74-6.79 (m, 3H), 7.20-7.25 (m, 1H).

To a solution of 1-[(2S)-2-azidopropyl]-3-methoxybenzene (11.0 g, 57.5 mmol, 1.0 equiv) in ethyl acetate (120 mL) was added Pd/C (1.0 g of 10 percent Pd) at room temperature. This reaction mixture was hydrogenated at 100 PSI in par shaker at room temperature for 20 h. After this time, catalyst was removed by filtration through celite, filtrate was concentrated under reduced pressure to get (2S)-1-(3-methoxyphenyl)propan-2-amine. LCMS (ES) m/z=166.2 [M+H]⁺. ¹H NMR (400 MHz, CDCl₃) δ ppm 1.13 (d, J=6.0 Hz, 3H), 2.44-2.54 (m, 1H), 2.67-2.72 (m, 1H), 3.16-3.21 (m, 1H), 3.79 (s, 3H), 6.74-6.78 (m, 3H), 7.21 (t, J=7.8 Hz, 1H). NH₂ Protons were not observed in ¹H NMR.

To a solution of 4-cyanobenzoic acid (3.62 g, 24.6 mmol, 1.1 equiv.) and (2S)-1-(3-methoxyphenyl)propan-2-amine (3.7 g, 22.4 mmol, 1.0 equiv.) in DMF (50 mL) was added DMAP (6.84 g, 56 mmol, 2.5 equiv.), stirred for 5 min and then EDC·HCl (8.59 g, 44.8 mmol, 2.0 equiv.) was added at 0° C. This reaction mixture was stirred at room temperature for 16 h. Progress of the reaction was monitored by TLC (30% ethyl acetate in n-Hexane). After this time, the reaction mixture was diluted with EtOAc (100 mL) and saturated sodium bicarbonate solution (100 mL). Organic layer was separated, washed with water (2×100 mL), 1N HCl solution (2×50 mL), saturated sodium bicarbonate solution (50 mL), brine solution (50 mL), dried over anhydrous sodium sulfate, filtered and concentrated under reduced pressure to get the desired product. The resulting residue was used in the next reaction without any further purification. LC-MS (ES) (m/z)=295.2 [M+H]⁺. ¹H NMR (400 MHz, CDCl₃) δ ppm 1.25 (d, J=6 Hz, 3H), 2.82-2.93 (m, 2H), 3.78 (s, 3H), 4.43-4.49 (m, 1H), 5.91 (d, J=6.4 Hz, 1H), 6.75-6.80 (m, 3H), 7.22 (t, J=8.4 Hz, 1H), 7.7 (d, J=8.0 Hz, 2H), 7.76 (d, J=8.0 Hz, 2H).

To stirred solution of 4-cyano-N-[(2S)-1-(3-methoxyphenyl)propan-2-yl]benzamide (5.8 g, 19.7 mmol, 1 equiv.) and 2-chloropyridine (5.59 mL, 59.1 mmol, 3.0 equiv.) in dichloromethane (100 mL) was added trifluoromethanesulfonic anhydride (9.93 mL, 59.1 mmol, 3.0 equiv.) via syringe slowly drop wise at −78° C. After 5 min, the reaction mixture was placed in an ice-water bath and warmed to 0° C. After 5 min, the resulting solution was allowed to warm to rt and stirred for 2 h. Progress of the reaction was monitored by TLC (20% EA in n-Hexane). Reaction mixture was cooled and quenched with aqueous sodium hydroxide solution (100 mL, 1N) to neutralize the trifluoromethanesulfonate salts. Dichloromethane (200 mL) was added to dilute the mixture and the layers were separated. The aqueous layer was extracted with DCM (150 mL). The combined organic layer was washed with brine (100 mL), was dried over anhydrous sodium sulfate, and was filtered. The volatiles were removed under reduced pressure to give the crude product as pale brown gum. The crude was purified by silica gel column chromatography using 8-10% EtOAc in hexane as an eluent to afford 4-[(3S)-6-methoxy-3-methyl-3,4-dihydroisoquinolin-1-yl]benzonitrile. LC-MS (ES) (m/z)=277.2 [M+H]⁺. ¹H NMR (400 MHz, CDCl₃) δ ppm 1.46-1.47 (m, 3H), 2.45-2.52 (m, 1H), 2.71-2.76 (m, 1H), 3.45 (s, 3H), 3.58-3.59 (m, 1H), 6.80 (d, J=8.0 Hz, 1H), 6.89 (d, J=8.4 Hz, 1H), 7.37-7.42 (m, 1H), 7.54 (d, J=8.0 Hz, 2H), 7.62 (d, J=7.2 Hz, 2H).

To a solution of 4-[(3S)-6-methoxy-3-methyl-3,4-dihydroisoquinolin-1-yl]benzonitrile (5.20 g, 18.8 mmol, 1.0 equiv.) in methanol (100 mL) was added sodium borohydride (2.02 g, 56.5 mmol, 3 equiv.) portion wise at 0° C. The suspension was stirred at room temperature for 1 h. Progress of the reaction was monitored by TLC (50% EtOAc in n-Hexane). After this time, the reaction mixture was concentrated under reduced pressure and obtained crude was diluted with EtOAc (150 mL) and water (50 mL). Organic layer was separated, washed with brine solution (50 mL), dried over anhydrous sodium sulfate, filtered and concentrated under reduced pressure to get the crude product (6.0 g). Obtained crude product was purified by flash column chromatography on silica gel using ethyl acetate in n-Hexane as an eluent. Desired products were eluted with 30-70% ethyl acetate in n-Hexane. Fractions containing products were combined and concentrated under reduced pressure to get 4-[(1S,3S)-6-methoxy-3-methyl-1,2,3,4-tetrahydroisoquinolin-1-yl]benzonitrile (600 mg, 11.45%) as yellow gum (over the time this became solid) (1.3 trans isomer) and 4-[(1R,3S)-6-methoxy-3-methyl-1,2,3,4-tetrahydroisoquinolin-1-yl]benzonitrile (1.3 cis isomer). (for Cis-isomer, Non Polar spot) LC-MS (m/z)=279.3 [M+H]⁺. (for Trans-isomer, Polar spot) LC-MS (m/z)=279.3 [M+H]⁺. ¹H NMR (400 MHz, CDCl₃) δ ppm 1.12 (d, J=6 Hz, 3H), 1.44-1.45 (m, 1H), 2.53-2.59 (m, 1H), 2.75-2.88 (m, 1H), 3.02 (bs, 1H), 3.80 (s, 3H), 5.22 (s, 1H), 6.65-6.69 (m, 2H), 6.76-6.78 (m, 1H), 7.25-7.28 (m, 2H), 7.56-7.58 (m, 2H).

To a stirred solution of 4-[(1S,3S)-6-methoxy-3-methyl-1,2,3,4-tetrahydroisoquinolin-1-yl]benzonitrile (80.0 mg, 287 μmol, 1.0 equiv), in dichloromethane (5 mL) was added triethylamine (0.101 mL, 0.719 mmol, 2.5 equiv), 3-(trimethylsilyl)propiolic acid (0.049 g, 0.34 mmol, 1.2 equiv) and followed by the addition of 2-Chloro-1-methylpyridinium iodide (0.088 g, 0.34 mmol, 1.2 equiv) at room temperature. The mixture was stirred for 1 h. The progress of the reaction was monitored by TLC (50% ethyl acetate in n-hexane). After completion of reaction, the mixture was diluted with dichloromethane (10 mL), washed with water (10 mL), brine (10 mL), dried over anhydrous sodium sulfate, filtered and concentrated under reduced pressure to obtain 4-[(1S,3S)-6-methoxy-3-methyl-2-[3-(trimethylsilyl)prop-2-ynoyl]-1,2,3,4-tetrahydroisoquinolin-1-yl]benzonitrile. LC-MS (ES) m/z. 403.2 [M+H]⁺

To a stirred solution of 4-[(1S,3S)-6-methoxy-3-methyl-2-[3-(trimethylsilyl)prop-2-ynoyl]-1,2,3,4-tetrahydroisoquinolin-1-yl]benzonitrile (0.100 g, 0.248 mmol, 1.0 eq), in dichloromethane (5 mL) and methanol (1.0 mL) was added potassium carbonate (0.210 g, 1.49 mmol, 6.0 eq) at 0° C. and the reaction was stirred at room temperature for 1 h. The progress of the reaction was monitored by TLC (30% ethyl acetate in n-hexane). After completion of reaction, the mixture was diluted with dichloromethane (80 mL), washed with water (10 mL), brine (10 mL), dried over anhydrous sodium sulfate, filtered and concentrated under reduced pressure to get the crude. The crude was purified by silica gel column chromatography using 15-20% ethyl acetate in n-hexane as an eluent to get the product which was repurified by preparative TLC using 30% ethyl acetate in n-hexane as the mobile phase. The product spot was isolated and lyophilized using ACN:Water (1:2) mixture for 16 h to afford 4-[(1S,3S)-6-methoxy-3-methyl-2-(prop-2-ynoyl)-1,2,3,4-tetrahydroisoquinolin-1-yl]benzonitrile. LC-MS (ES) m/z: 331.3 [M+H]⁺. HPLC purity: 99.89% at 254 nm. Chiral HPLC ee: 100% at 254.0. 1H NMR (400 MHz, DMSO-d6) δ ppm 0.884 (d, J=6 Hz, 1H), 1.01 (d, J=6 Hz, 2H), 1.21 (m, 1H), 2.56 (s, 1H), 2.64 (m, 1H), 2.862 (t, J=7.6, 3H), 3.119 (t, J=7.6, 1 H), 3.707 (d, J=4.8 Hz, 3H), 4.69 (s, 1H), 4.902 (s, 1H), 6.1 (s, 1H), 6.39 (s, 1H), 6.76-6.83 (m, 2H), 7.4-7.5 (m, 3H), 7.68 (d, J=8.4 Hz, 2H), 7.75 (d, J=7.6 Hz, 1H).

### Procedure 18: Synthesis of Compound B-1

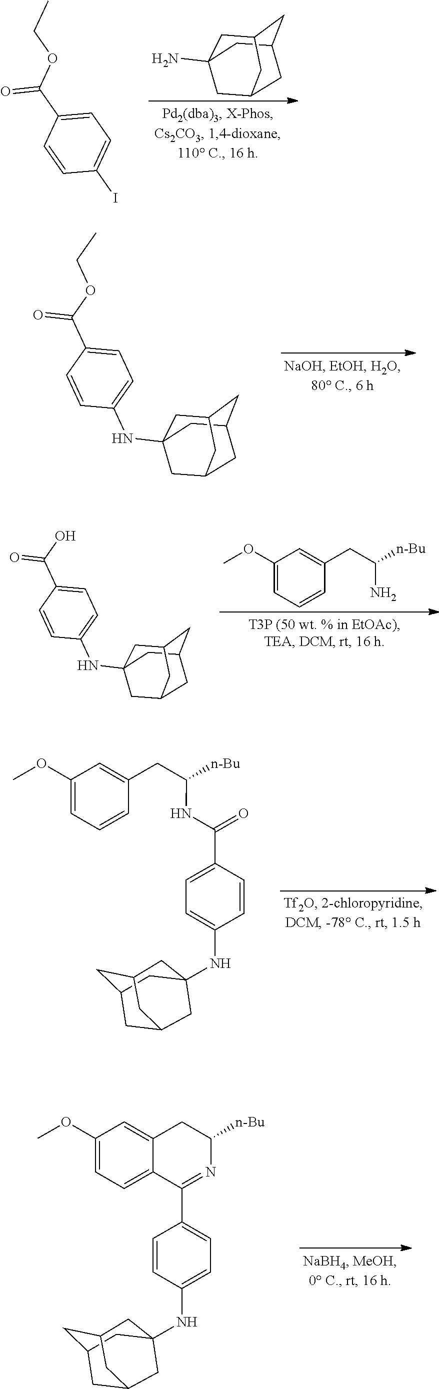

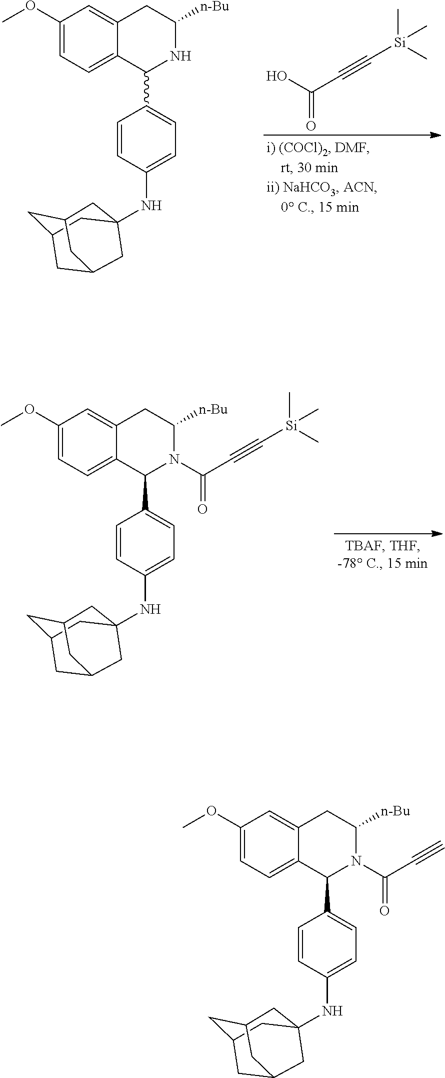

To a stirred solution of ethyl 4-iodobenzoate (6.0 g, 21.73 mmol, 1 eq) and (3s,5s,7s)-adamantan-1-amine (3.93 g, 26.08 mmol, 1.2 eq) in 1,4-dioxane was added XPhos (0.51 g, 1.08 mmol, 0.05 eq) and Cs₂CO₃ (14.26 g, 43.26 mmol, 2 eq) at room temperature. The reaction mixture was purged under argon for 15 mins, then added Pd₂(dba)₃ (0.516 g, 0.65 mmol, 0.03 eq) to the reaction and stirred at 110° C. for 16 h. The progress of the reaction was monitored by TLC (15% ethyl acetate in hexane). After completion of reaction, the reaction mixture was filtered through celite bed and the celite bed was washed with ethyl acetate (150 mL). The filtrate was concentrated under reduced pressure to obtain crude. Obtained crude product was purified by flash chromatography using ethyl acetate in hexane as an eluent to obtain ethyl 4-(((3s,5s,7s)-adamantan-1-yl)amino)benzoate. LC-MS (m/z)=300.0 [M+H]⁺. ¹H NMR (400 MHz, CDCl₃) δ ppm 1.34 (t, J=7.0 Hz, 3H), 1.67-1.74 (m, 6H), 1.97 (s, 6H), 2.14 (s, 3H), 4.29 (q, J=6.9 Hz, 2H), 6.67 (d, J=8.4 Hz, 2H), 7.80 (d, J=8.4 Hz, 2H).

To a solution of ethyl 4-(((3s,5s,7s)-adamantan-1-yl)amino)benzoate (1.6 g, 5.37 mmol, 1 eq) in EtOH (29 mL) and water (11 mL) was added sodium hydroxide (0.43 g, 10.70 mmol, 2 eq) and stirrer at 80° C. for 6 h. TLC (15% ethyl acetate in hexane) showed the reaction was completed. The reaction mixture was concentrated under reduced pressure and the obtained crude was acidified with 5% aqueous citric acid solution (_{P}H=4). Finally, the product was extracted with EtOAc (75 mL) from aqueous layer, dried over anhydrous Na₂SO₄, filtered and concentrated under reduced pressure to get the product 4-(((3s,5s,7s)-adamantan-1-yl)amino)benzoic acid. LC-MS (m/z)=272.0 [M+H]⁻. ¹H NMR (400 MHz, DMSO-d₆) δ ppm 1.61-1.68 (m, 6H), 1.91 (s, 6H), 2.06 (s, 3H), 5.87 (bs, 1H), 6.72 (d, J=8.8 Hz, 2H), 7.58 (d, J=8.8 Hz, 2H), 12.00 (bs, 1H).

To a solution of 4-(((3s,5s,7s)-adamantan-1-yl)amino)benzoic acid (1.05 g, 3.88 mmol, 1.2 eq) in DCM (20 mL) was added TEA (1.3 g, 12.92 mmol, 4 eq), stirred for 5 min and then T3P (50 wt. % in EtOAc) (1.53 g, 4.84 mmol, 1.5 eq) was added at 0° C. and stirred for another 5 mins. Then (S)-1-(3-methoxyphenyl)hexan-2-amine (0.67 g, 3.23 mmol, 1 eq) was added to the reaction mixture and then reaction mixture was stirred at room temperature for 16 h. The progress of the reaction was monitored by TLC (30% ethyl acetate in hexane). The reaction mixture was diluted with DCM (50 mL) and saturated sodium bicarbonate solution (20 mL) Organic layer was separated, washed with brine solution (20 mL), dried over anhydrous sodium sulfate, filtered and concentrated under reduced pressure to get the crude product. Obtained crude product was purified by flash chromatography using ethyl acetate in hexane as an eluent to get 4-(((3R,5R,7R)-adamantan-1-yl)amino)-N—((S)-1-(3-methoxyphenyl)hexan-2-yl)benzamide. LC-MS (m/z)=461.0 [M+H]⁺. ¹H NMR (400 MHz, DMSO-d₆) δ ppm 0.80 (bs, 3H), 1.22 (bs, 4H), 1.45 (bs, 2H), 1.64 (s, 6H), 1.89 (s, 6H), 2.05 (s, 3H), 2.65-2.76 (m, 2H), 3.16 (d, J=4.0 Hz, 1H), 3.66 (s, 3H), 4.07 (bs, 1H), 5.49 (s, 1H), 6.68-6.75 (m, 4H), 7.11-7.12 (m, 1H), 7.50 (d, J=7.2 Hz, 2H), 7.57-7.64 (m, 1H).

Trifluoromethanesulfonic anhydride (0.547 mL, 3.26 mmol, 3.0 eq) was added via syringe over a period of 1 min to a stirred mixture of 4-(((3R,5R,7R)-adamantan-1-yl)amino)-N—((S)-1-(3-methoxyphenyl)hexan-2-yl)benzamide (0.5 g, 1.08 mmol, 1 eq) and 2-chloropyridine (0.3 mL, 3.26 mmol, 3.0 eq) in dichloromethane (3.6 mL) at −78° C. After 5 min, the reaction mixture was placed in an ice-water bath and warmed to 0° C. After 5 min, the resulting solution was allowed to warm to 23° C. After 1 h, aqueous sodium hydroxide solution (5 mL, 1N) was introduced to neutralize the trifluoromethanesulfonate salts. Dichloromethane (50 mL) was added to dilute the mixture and the layers were separated. The organic layer was washed with brine (7 mL), was dried over anhydrous sodium sulfate, and was filtered. The volatiles were removed under reduced pressure to give the crude product. Obtained crude product was purified by flash chromatography using ethyl acetate in hexane as an eluent to get the desired product ((3R,5R,7R)—N-(4-((S)-3-butyl-6-methoxy-3,4-dihydroisoquinolin-1-yl)phenyl)adamantan-1-amine. LC-MS (m/z)=443.3 [M+H]⁺.

A solution of the (3R,5R,7R)—N-(4-((S)-3-butyl-6-methoxy-3,4-dihydroisoquinolin-1-yl)phenyl)adamantan-1-amine (0.5 g, 1.13 mmol, 1 eq) in methanol (9 mL) was added sodium borohydride (0.128 g, 33.93 mmol, 3 eq) at 0° C. The suspension was stirred at room temperature for 16 h. After this time, the reaction mixture was concentrated and obtained crude was diluted with EtOAc (30 mL) and water (10 mL). Organic layer was separated, washed with brine solution (10 mL), dried over anhydrous sodium sulfate, filtered and concentrated under reduced pressure to get the crude product. Obtained crude product was purified by flash chromatography using ethyl acetate in hexane as an eluent to get (3R,5R,7R)—N-(4-((1S,3S)-3-butyl-6-methoxy-1,2,3,4-tetrahydroisoquinolin-1-yl)phenyl)adamantan-1-amine. The isolated pure product was treated with metal scavenger Quadrasil AP (compound was dissolved in THF (5 mL) and quadrasil AP (50 mg) was added, the mixture was stirred for 0.5 h, filtered. This is repeated one more time and concentrated). LC-MS (m/z)=445.3 [M+H]⁺. ¹H NMR (400 MHz, CDCl₃) δ ppm 0.84-088 (m, 3H), 1.22-1.30 (m, 4H), 1.42-1.43 (m, 2H), 1.60-1.70 (m, 6H), 1.85 (s, 6H), 2.09 (bs, 3H), 2.53-2.60 (m, 1H), 2.82-2.86 (m, 1H), 2.87-2.97 (m, 1H), 3.78 (s, 3H), 5.13 (s, 1H), 6.66-6.70 (m, 4H), 6.84-6.90 (m, 3H).

To a solution of 3-(trimethylsilyl)propiolic acid (11.8 mg, 0.083 mmol, 1.0 eq) in DMF (0.24 mg, 0.003 mmol, 0.04 eq) was added oxalyl chloride (11.5 mg, 0.091 mmol, 1.1 eq) at room temperature and stirred for 30 minutes. After this time, reaction mixture was concentrated under reduced pressure to get 3-(trimethylsilyl)propioloyl chloride as colorless oil. This acid chloride was carried to next step without any further purification.

To a solution of (3R,5R,7R)—N-(4-((1S,3S)-3-butyl-6-methoxy-1,2,3,4-tetrahydroisoquinolin-1-yl)phenyl)adamantan-1-amine (37 mg, 0.083 mmol, 1.0 eq) in acetonitrile (1.0 mL) was added sodium bicarbonate (52.5 mg, 0.62 mmol, 7.5 eq) at 0° C. After stirring for 5 minutes, a solution of 3-(trimethylsilyl)propioloyl chloride in acetonitrile (1.0 mL) was added to the above reaction mass. The resulting mixture stirred at 0° C. for 15 min, progress of the reaction was monitored by TLC (70% ethyl acetate in n-Hexane). After this time, reaction mass was diluted with EtOAc (30 mL) and water (10 mL). Organic layer was separated, washed with brine solution (10 mL), dried over anhydrous sodium sulfate, filtered and concentrated under reduced pressure to get the crude product. This crude product was carried to next step without any further purification. LC-MS (m/z)=569.4 [M+H]⁺

To a solution of 1-((1S,3S)-1-(4-(((3R,5R,7R)-adamantan-1-yl)amino)phenyl)-3-butyl-6-methoxy-3,4-dihydroisoquinolin-2(1H)-yl)-3-(trimethylsilyl)prop-2-yn-1-one (48 mg, 0.084 mmol, 1.0 eq) in THF (1.5 mL) was added TBAF ((M solution in THF) (0.092 mL, 0.092 mmol, 1.1 eq) at −78° C. This reaction mixture was stirred at −78° C. for 15 minutes. Progress of the reaction was monitored by TLC (25% ethyl acetate in n-Hexane). After this time, the reaction mixture was quenched with saturated aqueous NaHCO₃ solution (5 mL) and product was extracted with ethyl acetate (25 mL). Organic layers were dried over anhydrous sodium sulfate, filtered and concentrated under reduced pressure. Obtained crude product was purified by preparative TLC using 25% ethyl acetate in n-Hexane as an eluent to get 1-((1S,3S)-1-(4-(((3R,5R,7R)-adamantan-1-yl)amino)phenyl)-3-butyl-6-methoxy-3,4-dihydroisoquinolin-2(1H)-yl)prop-2-yn-1-one. LC-MS (m/z)=497.3 ([M+H]⁺. ¹H NMR (400 MHz, DMSO-d6) δ ppm 0.79-0.80 (m, 3H), 1.18-1.24 (m, 6H), 1.51 (bs, 1H), 1.61 (s, 5H), 1.79 (s, 6H), 2.01 (s, 3H), 2.71-2.83 (m, 1H), 3.00-3.09 (m, 2H), 3.72-3.73 (m, 3H), 4.15 (bs, 0.3H), 4.40 (bs, 1H), 4.61 (bs, 0.7H), 5.95 (s, 0.5H), 6.16 (s, 0.5H), 6.59-6.66 (m, 2H), 6.76-6.85 (i, 4H), 7.29 (d, J=8.4 Hz, 0.5H), 7.40 (d, J=7.6 Hz, 0.5H).

### Compound B-42 and Compound B-22 and were Synthesized by Following the Synthesis Procedure as Described Above

| | | | LCMS m/z | |
|---|---|---|---|---|
| No. | Structure | Name | [M + H]⁺ | ¹H-NMR |
| B-42 | 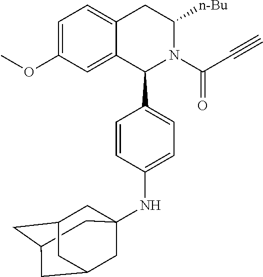 | 1-((1S,3S)-1-(4- (((3R,5R,7R)- adamantan-1- yl)amino)phenyl)-3- butyl-7-methoxy-3,4- dihydroisoquinolin- 2(1H)-yl)prop-2-yn-1- one | 496.7 | ¹H NMR (400 MHz, DMSO- d6) δ ppm 0.78 − 0.83 (m, 3 H), 1.31 − 1.47 (m, 5 H), 1.59 (s, 6 H), 1.78 (s, 6 H), 2.00 (s, 3 H), 2.65 − 2.80 (m, 1 H), 2.97 − 3.1 (m, 1 H), 3.72 − 3.77 (m, 3 H), 4.31 − 4.40 (m, 1 H), 4.57 − 4.60 (m, 1 H), 4.80 − 5.00 (m, 1 H), 5.93 (s, 0.57 H), 6.20 (s, 0.42 H), 6.58 − 6.65 (m, 2 H), 6.74 − 6.89 (m, 3 H), 7.04 − 7.18 (m, 2 H) |
| B-22 | 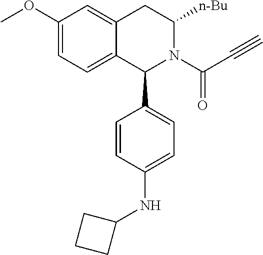 | 1-((1S,3S)-3-butyl-1-(4- (cyclobutylamino) phenyl)-6-methoxy-3,4- dihydroisoquinolin- 2(1H)-yl)prop-2-yn-1- one | 417.3 | ¹HNMR(400 MHz, DMSO- d6) δ ppm0.78 − 0.81 (m, 3 H), 1.17 − 1.20 (m, 5 H), 1.46 (s, 1 H), 1.67 -1.69 (m, 4 H), 3.01 − 3.04 (m, 0.5 H), 3.69 − 4.60 (m, 1 H), 5.68 − 5.81 (m, 3.70 (m, 4 H), 4.30 (s, 0.5 H), 4.40 − 4.41 (m, 0.5 H), 4.57 − 2.22 − 2.24 (m, 2 H), 2.65 (s, 1 H), 2.79 2.83 (m, 0.5 H), 1 H), 5.90 (s, 0.5 H), 6.13 (s, 0.5 H), 6.30 − 6.37(m, 2 H), 6.74 − 6.86 (m, 4 H), 7.30 - 7.32 (m, 0.5 H), 7.44-746 (m, 0.5 H). |

### Procedure 19: Synthesis of Compound B-20

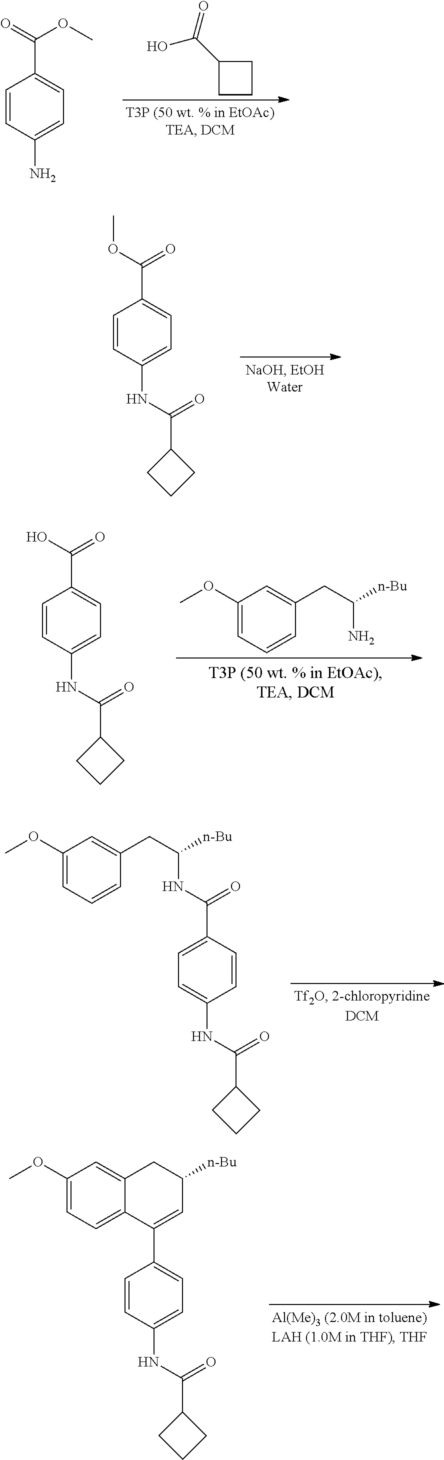

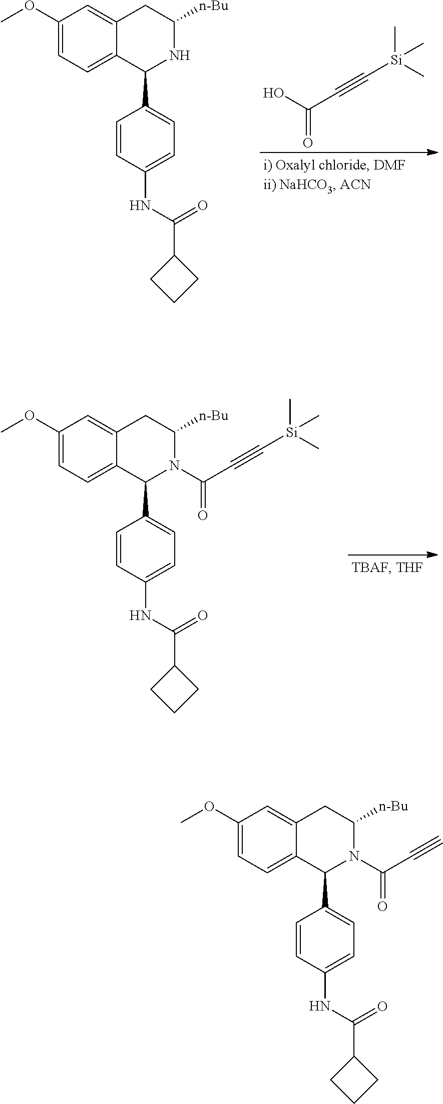

To a solution of cyclobutanecarboxylic acid (3.64 g, 36.4 mmol, 1.1 eq) in DCM (15 mL) was added TEA (18.4 mL, 132 mmol, 4 eq.), stirred for 5 min and then T3P (50 wt. % in EtOAc) (15.0 mL, 49.6 mmol, 1.5 eq) was added at 0° C. and stirred for another 30 min. Then a solution of Methyl 4-aminobenzoate (5.0 g, 33.1 mmol, 1 eq) in DCM (10 mL) was added to the reaction mixture at 0° C. and then reaction mixture was stirred at room temperature for 16 h. Progress of the reaction was monitored by TLC (30% ethyl acetate in n-Hexane). After this time, the reaction mixture was diluted with DCM (2×100 mL) and saturated sodium bicarbonate solution (15 mL). Organic layer was separated, washed with water (10 mL), brine solution (10 mL), dried over anhydrous sodium sulfate, filtered and concentrated under reduced pressure to get methyl 4-cyclobutaneamidobenzoate. LCMS (ES) m/z=234.1 [M+H]+. ¹H NMR (400 MHz, DMSO-d₆) δ ppm 1.77-1.97 (m, 2H), 2.07-2.17 (m, 2H), 2.22-2.47 (m, 2H), 3.10-3.22 (m, 1H), 3.78 (s, 3H), 7.10 (d, J=8.0 Hz, 2H), 7.86 (d, J=8.4 Hz, 2H), 10.05 (s, 1H).

To a solution of methyl 4-cyclobutaneamidobenzoate (3.50 g, 15 mmol, 1 eq) in EtOH (15.0 mL) and water (7.5 mL) was added sodium hydroxide (1.20 g, 30.0 mmol, 2 eq) at room temperature and the reaction mixture was stirred at 80° C. for 16 h. Progress of the reaction was monitored by TLC (30% ethyl acetate in Hexane). After completion of the reaction, the reaction mixture was concentrated under reduced pressure to remove ethanol from reaction mass and the remaining aqueous layer was extracted with EtOAc (20 mL). Finally, the aqueous layer was acidified with 5% citric acid (pH˜4) and then product was extracted with EtOAc (2×80 mL). Organic layer was dried over anhydrous sodium sulfate, filtered and concentrated under reduced pressure to get the product which was triturated with n-pentane (10 mL) for 5 minutes, decanted the pentane layer and dried under high vacuum to afford 4-cyclobutaneamidobenzoic acid. LCMS (ES) m/z=220.3 [M+H]+

To a solution of 4-cyclobutaneamidobenzoic acid (2.12 g, 9.65 mmol, 1 eq) in DCM (15 mL) under nitrogen atmosphere was added triethylamine (5.42 mL, 38.6 mmol, 4 eq) at 0° C., stirred for 10 mins and then propanephosphonic acid anhydride (50 wt. % in ethyl acetate) (4.6 g, 14.5 mmol, 1.5 eq) was added at 0° C. to the reaction mixture, stirred at 0° C. for 15 mins and then (2S)-1-(3-methoxyphenyl)hexan-2-amine (2 g, 9.65 mmol, 1 eq) dissolved in DCM (5 mL) was added to the reaction mixture at 0° C. and then the reaction mixture was stirred at room temperature for 16 h. TLC (50% EtOAc in hexane) showed the reaction was completed after 16 h. The reaction mixture was diluted with DCM (80 mL), washed with saturated sodium bicarbonate solution (20 mL) and water (10 mL). Organic layer was dried over anhydrous Na₂SO₄, filtered and concentrated under reduced pressure to get 4-cyclobutaneamido-N-[(2S)-1-(3-methoxyphenyl)hexan-2-yl]benzamide. LCMS (ES) m/z=409.3 [M+H]⁺.

To a solution of 4-cyclobutaneamido-N-[(2S)-1-(3-methoxyphenyl)hexan-2-yl]benzamide (0.1 g, 0.245 mmol, 1 eq) in DCM (5 mL) was added 2-chloropyridine (0.046 mL, 0.490 mmol, 2 eq) at room temperature and the reaction mixture was cooled to −78° C. and trifluoromethanesulfonyl trifluoromethanesulfonate (0.082 mL, 0.49 mmol, 2 eq) was added to the mixture at −78° C. and stirred. After 10 min, reaction mixture was placed in an ice-water bath and warmed to 0° C. for 10 min. Then resulting solution was allowed to warm to room temperature and stirred for 1 h. Progress of the reaction was monitored by TLC (70% EtOAc in n-hexane). After completion of the reaction, reaction mixture was quenched with 1 M NaOH (5 mL) at 0° C. and then diluted with DCM (5 mL) and extracted with DCM (25 mL) and washed with water (5 mL). Organic layer was dried over anhydrous sodium sulfate, filtered and concentrated under reduced pressure to afford (S)—N-(4-(3-butyl-6-methoxy-3,4-dihydroisoquinolin-1-yl)phenyl)cyclobutanecarboxamide. LCMS (ES) m/z=391.3 [M+H]+

To a solution of N-{4-[(3S)-3-butyl-6-methoxy-3,4-dihydroisoquinolin-1-yl]phenyl}cyclobutanecarboxamide (1.40 g, 3.58 mmol, 1 eq) in MeOH (20 mL) under nitrogen atmosphere was added sodium borohydride (0.396 g, 10.8 mmol, 3 eq) at 0° C. and then the reaction mixture was stirred at room temperature for 1 h. Reaction was monitored by TLC (70% EtOAc in hexane) and LC-MS. After completion of the reaction, reaction mixture was quenched with acetone and concentrated under reduced pressure then extracted with EtOAc (50 mL) and washed with water (10 mL). Organic layer was dried over anhydrous sodium sulfate, filtered and concentrated under reduced pressure to get crude product. Obtained crude product was purified by flash column chromatography with 45-55% ethyl acetate in hexane as an eluent. Fractions containing product was combined and concentrated under reduced pressure to get a crude of cis and trans mixture. Again the crude was purified by preparative TLC using 60% ethyl acetate in n-Hexane as eluent to get N-{4-[(1S,3S)-3-butyl-6-methoxy-1,2,3,4-tetrahydroisoquinolin-1-yl]phenyl}cyclobutanecarboxamide. LCMS (ES) m/z=393.3 [M+H]+

To a solution of 3-(trimethylsilyl)propiolic acid (0.1 g, 0.703 mmol, 1 eq) in DMF (0.002 mL, 0.028 mmol, 0.04 eq) was added oxalyl chloride (0.072 mL, 0.844 mmol, eq) at room temperature and stirred for 30 minutes. Then reaction mixture was concentrated under reduced pressure to get 3 (trimethylsilyl)propioloyl chloride.

To a solution of N-{4-[(1S,3S)-3-butyl-6-methoxy-1,2,3,4-tetrahydroisoquinolin-1-yl]phenyl}cyclobutanecarboxamide (0.2 g, 0.509 mmol, 1 eq) in acetonitrile (7.0 mL) was added sodium bicarbonate (0.325 g, 3.82 mmol, 7.5 eq) at 0° C. After stirring for 5 minutes, a solution of 3-(trimethylsilyl)propioloyl chloride (0.123 g, 0.764 mmol, 1.5 equiv.) in acetonitrile (3 mL) was added to the above reaction mass at 0° C. The resulting mixture was stirred at 0° C. for 45 min, progress of the reaction was monitored by TLC (25% ethyl acetate in n-Hexane). After this time, reaction mass was diluted with EtOAc (20 mL) and water (5 mL). Organic layer was separated, washed with brine solution (7.0 mL), dried over anhydrous sodium sulfate, filtered and concentrated under reduced pressure to get the crude product. LCMS (ES) m/z=517.3 [M+H]+.

To a solution of N-{4-[(1S,3S)-3-butyl-6-methoxy-2-[3-(trimethylsilyl)prop-2-ynoyl]-1,2,3,4-tetrahydroisoquinolin-1-yl]phenyl}cyclobutanecarboxamide (0.2 g, 0.387 mmol, 1 eq) in THF (5 mL) was added TBAF (1 M solution in THF) (0.968 mL, 0.968 mmol, 2.5 eq) at −78° C. This reaction mixture was stirred at −78° C. for 15 minutes. Progress of the reaction was monitored by TLC (25% ethyl acetate in n-Hexane). After this time, the reaction mixture was quenched with saturated aqueous NaHCO₃ solution (10 mL) at −78° C. and product was extracted with ethyl acetate (2×50 mL). Combined organic layers was dried over anhydrous sodium sulfate, filtered and concentrated under reduced pressure. Obtained crude product was purified by preparative TLC using 30% ethyl acetate in n-hexane as an eluent. Product fraction was collected and concentrated under reduced pressure to get N-{4-[(1S,3S)-3-butyl-6-methoxy-2-(prop-2-ynoyl)-1,2,3,4-tetrahydroisoquinolin-1-yl]phenyl}cyclobutanecarboxamide. LCMS (ES) m/z=445.2 [M+H]⁺. 1H NMR (400 M-Z, DMSO-d6) δ ppm: 0.65-0.68 (m, 3H), 0.75 (bs, 1H), 1.09-1.11 (m, 4H), 1.25 (s, 1H), 1.54 (s, 1H), 1.78-1.86 (m, 1H), 1.89-1.93 (m, 1H), 2.05-2.07 (bs, 2H), 2.20-2.25 (m, 2H), 2.63-2.66 (in, 0.5H), 2.99-3.01 (in, 0.5H), 3.13-3.18 (m, 1H), 3.74-3.76 (s, 3H), 4.04 (s, 0.5H), 4.53-4.59 (i, 1.5H), 6.50-6.55 (1, 1H), 6.77-6.79 (n, 0.5H), 6.84 (s, 1H), 6.90-6.94 (m, 2H), 7.03 (d, J=8.0 Hz, 1H), 7.26 (d, J=8.4 Hz, 0.5H), 7.47-7.54 (m, 2H), 9.67-9.72 (m, 1H).

### Compound B-19 was Synthesized by Following the Synthetic Route as Described for Compound B-20

| | | | LCMS m/z | |
|---|---|---|---|---|
| No. | Structure | Name | [M + H]⁺ | ¹H-NMR |
| B-19 | 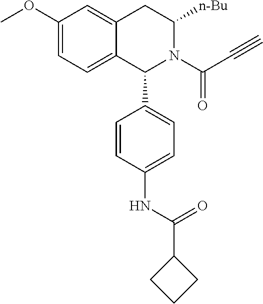 | N-(4-((1R,3S)-3- butyl-6-methoxy-2- propioloyl-1,2,3,4- tetrahydroisoquinolin- 1-yl)phenyl) cyclobutane carboxamide | 445.2 | ¹H NMR (400 MHZ, DMSO- d6) δ ppm : 0.65 − 0.68 (m, 3 H), 0.85 (bs, 1 H), 1.09 − 1.11 (m, 4 H), 1.54 (s, 1 H), 1.54 (s, 1 H), 1.78 − 1.86 (m, 1 H), 1.89 − 1.93 (m, 1 H), 2.15 − 2.17 (m, 3 H), 2.63 − 2.66 (m, 0.5 H), 2.99 − 3.03 (m, 0.5 H), 3.13 − 3.18 (m, 2 H), 3.74 − 3.76 (m, 3 H), 4.04 (s, 0.5 H), 4.53 − 4.59 (m, 1.5 H), 6.50 − 6.55 (m, 1 H), 6.77 − 6.79 (m, 0.5 H), 6.84 (s, 1 H), 6.90 − 6.94 (m, 2 H), 7.02 − 7.04 (m, 1 H), 7.25 − 7.27 (m, 0.5 H), 7.47 − 7.54 (m, 2 H), 9.67 − 9.72 (m, 1 H). |

### Procedure 20: Synthesis of Compound B-49

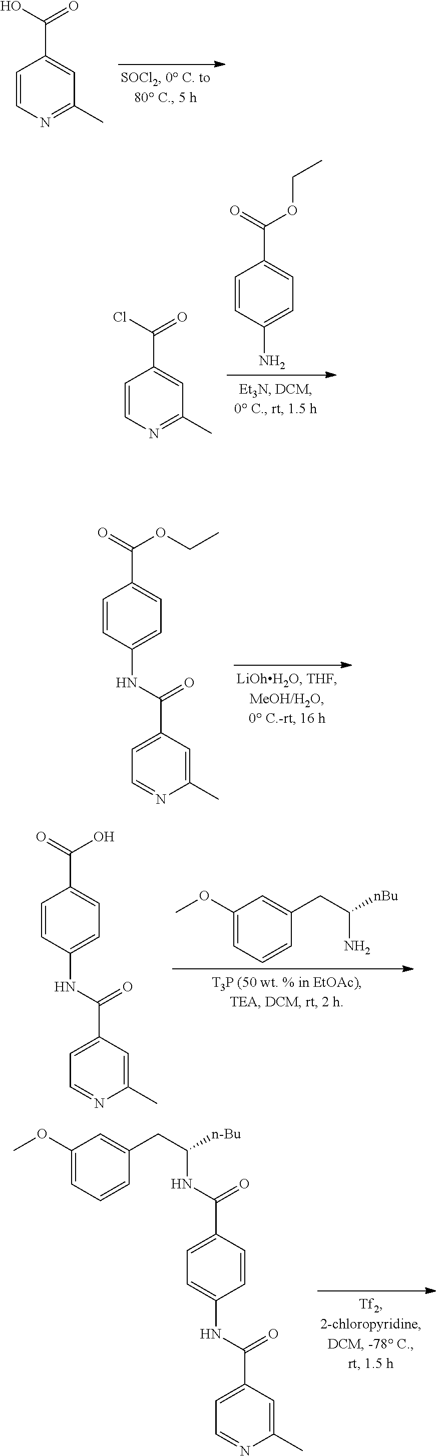

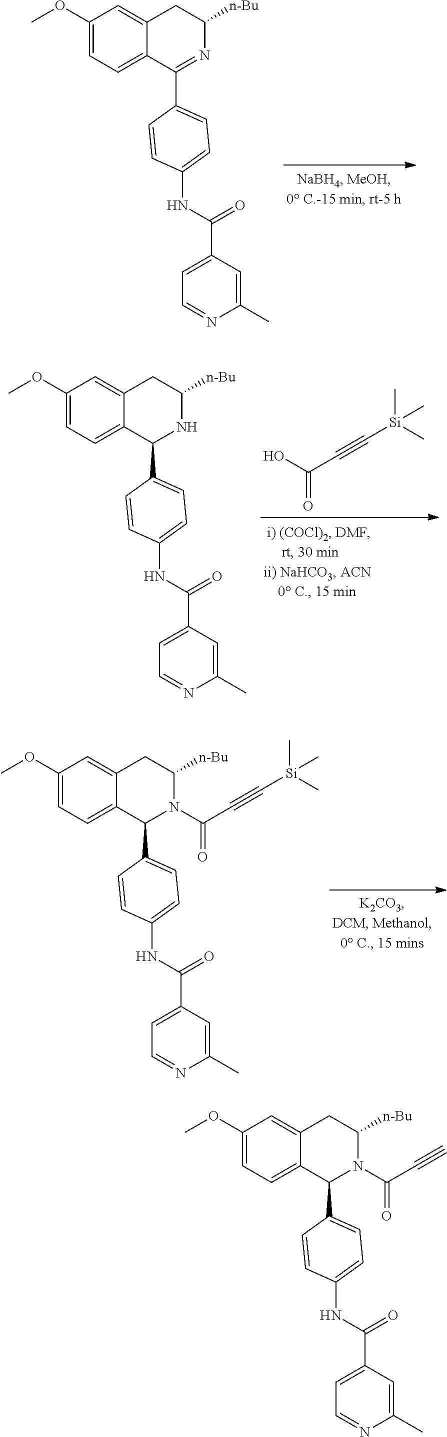

To a solution of 2-methylpyridine-4-carboxylic acid (3.00 g, 21.9 mmol, 1.0 eq) in N,N-dimethylformamide (0.067, 0.87 mmol, 0.04 eq) was added SOCl₂ (40.0 mL) at 0° C., This mixture was refluxed for 3 h at 80° C., and the excess of thionyl chloride was concentrated under reduced pressure to get 2-methylpyridine-4-carbonyl chloride. This crude product carried to next step without any further purification.

To a stirred solution of ethyl 4-aminobenzoate (3.19 g, 19.3 mmol, 1, 1.0 eq) in DCM (40.0 mL) was added triethylamine (15.0 mL, 116 mmol, 6 eq) 0° C. and the reaction was stirred for 15 mins. Then 2-methylpyridine-4-carbonyl chloride (20 ml DCM) (3.00 g, 19.3 mmol, 1 eq) was added slowly. After this time, the reaction was stirred at room temperature for 12 h. After completion of the reaction, the reaction slowly quenched with water, diluted with DCM (100 mL), stirred at room temperature for 5 mins. Then the layers were separated. Aqueous layer was extracted with DCM (2×100.0 mL). Combined organic layer was washed with water (50.0 mL), separated the layers. Then the organic layer was dried over anhydrous sodium sulfate, filtered and concentrated under reduced pressure to get the crude. The crude was purified by column 10% MeOH/DCM as an eluent to afford ethyl 4-(2-methylpyridine-4-amido)benzoate. LC-MS (ES) m/z: 285.2 [M+H]⁺. 1H NMR (400 MHz, DMSO-d6): δ (ppm) 10.72 (s, 1H), 8.61 (d, J=5.2 Hz, 1H), 7.97-7.89 (m, 4H), 7.76 (s, 1H), 7.62 (d, J=4.8 Hz, 1H), 4.30-4.25 (m, 2H), 2.48 (s, 3H), 1.31-1.28 (m, 3H).

To a stirred solution of ethyl 4-(2-methylpyridine-4-amido)benzoate (2.50 g, 8.79 mmol, 1.0 eq) in MeOH (20.0 mL), THF (20.0 mL) and Water (15.0 mL) was added LiOH·H2O (0.76 g, 17.6 mmol, 2.0 eq) and the reaction was stirred at rt for 16 h. Reaction was monitored by TLC (5% MeOH/DCM). After this time reaction mixture was concentrated under reduced pressure at rt and the aqueous layer was extracted with EtOAc (2×50 mL). The aqueous layer was then acidified with 5% citric acid (pH=5), Compound precipitate as brownish solid, filter the solid and dried under reduced pressure to get the 4-(2-methylpyridine-4-amido)benzoic acid. LC-MS (ES) (m/z)=257.2 [M+H]⁺. 1H NMR (400 MHz, DMSO-d6) δ ppm 12.71 (s, 1H), 10.69 (s, 1H), 8.63 (d, J=4.8 Hz, 1H), 7.95-7.87 (m, 4H), 7.71 (s, 1H), 7.64-7.57 (m, 1H).

To a solution of (2S)-1-(3-methoxyphenyl)hexan-2-amine (1.0 g, 4.82 mmol, 1.0 eq), 4-(2-methylpyridine-4-amido)benzoic acid (1.48 g, 5.79 mmol, 1.2 eq) in DCM (20.0 mL) was added Triethylamine (2.69 mL, 19.3 mmol, 4 eq), stirred for 5 min and then T3P (50 wt. % in EtOAc) (2.15 ml, 7.24 mmol, 1.5 eq) was added at 0° C. and stirred for another 30 min. Progress of the reaction was monitored by TLC (5% MeOH/DCM). After the completion of the reaction, the reaction mixture was quenched with water (20.0 mL) and extracted with DCM (2×25 mL). The combined organic extracts were washed with water (15 mL) and brine solution (15 mL) and dried over anhydrous Na₂SO₄, concentrated under reduced pressure to get the crude compound. The crude was purified by flash column chromatography using 3-5% methanol in DCM as an eluent to yield ethyl (S)—N-(4-((1-(3-methoxyphenyl)hexan-2-yl)carbamoyl)phenyl)-2-methylisonicotinamide. LC-MS (m/z)=446.3 [M+H]⁺. 1H NMR (400 MHz, DMSO-d6) δ ppm: 0.82 (d, J=6.8 Hz, 3H), 1.13-1.31 (m, 5H), 1.50 (d, J=6.0 Hz, 2H), 2.65 (s, 3H), 2.71-2.81 (m, 2H), 3.67 (s, 3H), 4.08-4.15 (m, 1H), 6.69 (d, J=8.8 Hz, 1H), 6.78 (d, J=6.4 Hz, 2H), 7.13 (t, J=8.0 Hz, 1H), 7.63 (d, J=4.8 Hz, 1H), 7.72 (s, 1H), 7.80 (s, 4H), 8.09 (d, J=8.8 Hz, 1H), 8.62 (d, J=5.2 Hz, 1H), 10.59 (s, 1H).

To stirred solution of (S)—N-(4-((1-(3-methoxyphenyl)hexan-2-yl)carbamoyl)phenyl)-2-methylisonicotinamide (1.2 g, 2.69 mmol, 1 eq) and 2-chloropyridine (1.02 mL, 10.8 mmol, 4.0 eq) in dichloromethane (25.0 mL) was added trifluoromethanesulfonic anhydride (1.81 mL, 10.8 mmol, 4.0 eq) slowly dropwise at −78° C. After 5 min, the reaction mixture was placed in an ice-water bath and warmed to 0° C. and the resulting solution was allowed to stir at 0° C. After 1.5 h, reaction was quenched with aqueous 1.0N sodium hydroxide solution (15 mL) to neutralize the trifluoromethanesulfonate salts. Dichloromethane (2×15 mL) was added to dilute the reaction mixture and the layers were separated. The combined organic layer was washed with brine (5 mL), dried over anhydrous sodium sulfate, and was filtered. The volatiles were removed under reduced pressure to give the crude product of (S)—N-(4-(3-butyl-6-methoxy-3,4-dihydroisoquinolin-1-yl)phenyl)-2-methylisonicotinamide. LC-MS(ES)m/z: 428.3 [M+H]+.

To a solution of N-{4-[(3S)-3-butyl-6-methoxy-3,4-dihydroisoquinolin-1-yl]phenyl}-2-methylpyridine-4-carboxamide (260 mg, 608 μmol), 1.0 eq) in methanol (10 mL) was added sodium boranuide (69.0 mg, 3 eq., 1.82 mmol, 3 eq) portion wise at 0° C. The suspension was stirred at room temperature for 1 h. Progress of the reaction was monitored by TLC (5% methanol in DCM). After this time, the reaction mixture was concentrated and obtained crude was diluted with EtOAc (20 mL) and water (10 mL). Organic layer was separated, washed with brine solution (10 mL), dried over anhydrous sodium sulfate, filtered and concentrated under reduced pressure to get the crude product. Obtained crude product was purified by Preparative TLC using 5% Methanol in DCM. Product fraction was collected and concentrated under reduced pressure to get tert-butyl N-{bicyclo[1.1.1]pentan-1-yl}-N-({4-[(1S,3S)-3-butyl-6-methoxy-1,2,3,4-tetrahydroisoquinolin-1-yl]phenyl}methyl)carbamate.

To a solution of 3-(trimethylsilyl)prop-2-ynoic acid (20 mg, 0.0141 mmol, 1.0 eq) in DMF (0.00043 mL, 0.00562 mmol, 0.04 eq) was added oxalyl chloride (0.013 mL, 0.155 mmol, 1.1 eq) at room temperature and stirred for 30 minutes. After this time, reaction mixture was concentrated under reduced pressure to get 3-(trimethylsilyl)prop-2-ynoyl chloride. This acid chloride was carried forward to the next step without any further purification.

To a solution of N-(4-((1S,3S)-3-butyl-6-methoxy-1,2,3,4-tetrahydroisoquinolin-1-yl)phenyl)-2-methylisonicotinamide (40 mg, 0.093 mmol, 1.0 eq) in acetonitrile (4.0 mL) was added sodium bicarbonate (59.4 mg, 0.698 mmol, 7.5 eq) at 0° C. After stirring for 5 minutes, a solution of 3-(trimethylsilyl)prop-2-ynoyl chloride (22.4 mg, 0.140 mmol, 1.5 eq) in acetonitrile (2.0 mL) was added to the above reaction mass at 0° C. The resulting mixture was stirred at 0° C. for 15 min. Progress of the reaction was monitored by TLC (50% ethyl acetate in n-Hexane). After this time, reaction mass was diluted with EtOAc (20 mL) and water (10 mL). Organic layer was separated, washed with brine solution (7.0 mL), dried over anhydrous sodium sulfate, filtered and concentrated under reduced pressure to get the crude of N-(4-((1S,3S)-3-butyl-6-methoxy-2-(3-(trimethylsilyl)propioloyl)-1,2,3,4-tetrahydroisoquinolin-1-yl)phenyl)-2-methylisonicotinamide. LC-MS (ES)m/z: 554.4 [M+H]+

To a solution of N-(4-((1S,3S)-3-butyl-6-methoxy-2-(3-(trimethylsilyl)propioloyl)-1,2,3,4-tetrahydroisoquinolin-1-yl)phenyl)-2-methylisonicotinamide (0.14 g, 0.253 mmol, 1.0 eq) in Dichloromethane (10.0 mL) and Methanol (2.0 mL) was added dipotassium carbonate (0.213 g, 1.52 mmol, 6.0 eq) at 0° C. This reaction mixture was stirred at 0° C. for 30 minutes. Progress of the reaction was monitored by LC-MS. After completion of starting material, the reaction mixture was diluted with Dichloromethane (2×10.0 mL) and separated with water (10.0 mL) and the combined organic layer was dried over anhydrous sodium sulfate, filtered and concentrated under reduced pressure to get the crude compound. Obtained crude product was purified by prep HPLC purification method by using following analytical condition to afford N-(4-((1S,3S)-3-butyl-6-methoxy-2-propioloyl-1,2,3,4-tetrahydroisoquinolin-1-yl)phenyl)-2-methylisonicotinamide. Analytical Conditions: Column: X-BridgeC-18 (250 mm×4.6 mm×5 mic); Mobile phase (A): 0.1% Ammonia in water; Mobile phase (B): Acetonitrile; Flow rate: 1.0 mL/min Gradient B: 0/10, 12/60, 22/95, 25/95, 27/10, 30/10. LC-MS (ES) (m/z)=482.5 [M+H]⁺. 1H NMR (400 MHz, DMSO-d6) δ ppm: 0.81 (t, J=6.8 Hz, 3H), 1.22 (bs, 5H), 1.50 (bs, 2H), 2.53 (s, 3H), 2.79 (s, 1H), 3.12 (s, 1H), 3.70 (d, J=5.6 Hz, 2H), 4.30 (s, 1H), 4.59 (s, 1H), 6.03 (s, 1H), 6.28 (s, 1H), 6.80 (t, J=8.0 Hz, 1H), 7.21 (t, J=8.4 Hz, 2H), 7.41 (d, J=7.6 Hz, 1H), 7.57 (d, J=8.4 Hz, 2H), 7.64 (d, J=8.8 Hz, 2H), 8.58 (d, J=4.8 Hz, 1H), 10.32 (s, 1H).

### Compound B-21 was Synthesized by Following the Procedure for Synthesis of Compound B-49 as Described Above

| | | | LCMS m/z | |
|---|---|---|---|---|
| No. | Structure | Name | [M + H]⁺ | ¹H-NMR |
| B-21 | 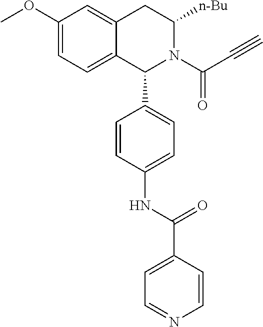 | N-(4-((3S)-3-butyl-6- methoxy-2-propioloyl- 1,2,3,4- tetrahydroisoquinolin-1- yl)phenyl)isonicotinamide | 468.5 | ¹H NMR (400 MHz, DMSO- d6) δ ppm: 0.80 − 0.81 (m, 3 H), 1.21 (bs, 5 H), 1.49 (s, 1 H), 2.79 − 2.89 (m, 1.5 H), 3.09 − 3.13 (m, 0.5 H), 3.70 − 3.71 (m, 3 H), 4.33 (s, 0.5 H), 4.54 (s, 1 H), 4.62 (s, 0.5 H), 4.71 (s, 1 H), 6.03 (s, 0.5 H), 6.29 (s, 0.5 H), 6.77 − 6.84 (m, 2 H), 7.22 − 7.24 (m, 2 H), 7.42 (d, J = 7.6 Hz, 1 H), 7.57 − 7.59 (m, 2 H), 7.64 − 7.66 (m, 1 H), 8.74 (s, 2 H), 10.34 − 10.45 (m, 1 H). |

### Procedure 21: Synthesis of Compound B-9

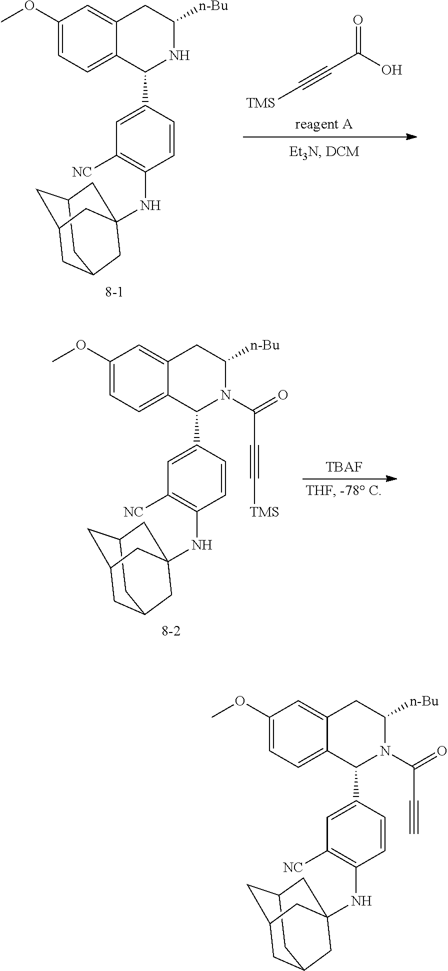

To a solution of 3-trimethylsilylprop-2-ynoic acid (32.10 mg, 225.70 μmol, 1 eq) in DCM (3 mL) was added 2-chloro-1-methyl-pyridin-1-ium; iodide (57.66 mg, 225.70 μmol, 1 eq). The reaction was stirred at 25° C. for 0.5 hr. Then the mixture was added dropwise the solution of Compound 8-1 and Et₃N (22.84 mg, 225.70 μmol, 31.41 uL, 1 eq) in DCM (10 mL) at 0° C. The reaction was stirred at 0° C. for 1 hr to give yellow solution. LCMS and TLC (PE/EtOAc=3/1) showed the reaction was completed. The reaction mixture was quenched H₂O (10 mL) and extracted with DCM (5 mL*3). The organic layers were washed with HCl (0.5N, 5 mL), then washed with Sat·NaHCO₃ (10 mL). The organic layers were dried over Na₂SO₄ and concentrated to give the crude product. The crude product was purified by column chromatography (SiO₂, Petroleum ether/Ethyl acetate=100/0 to 90/10) to give Compound 8-2. ¹H NMR (400 MHz, CHLOROFORM-d) δ ppm 7.38 (dd, J=9.07, 2.06 Hz, 1H) 6.90-7.16 (m, 3H) 6.71-6.86 (m, 2H) 6.51-6.66 (m, 1H) 4.58-4.69 (m, 1H) 4.32-4.48 (m, 1H) 3.78-3.88 (m, 3H) 2.93-3.19 (m, 1H) 2.38-2.72 (m, 1H) 2.15 (br s, 3H) 1.98 (br s, 6H) 1.67-1.75 (m, 6H) 1.26 (s, 2H) 1.01-1.21 (m, 4H) 0.71-0.82 (m, 3H) 0.18-0.30 (m, 9H).

To a solution of Compound 8-2 (42 mg, 70.72 μmol, 1 eq) in THF (5 mL) were added TBAF (1 M, 77.79 uL, 1.1 eq) at −78° C. The mixture was stirred at −78° C. for 20 mins to give a white solution. LCMS and TLC (PE:EtOAc=3:1) showed the mixture was completed. The mixture was added H₂O (5 mL), extracted with EtOAc (8 mL*3). The organic layers were dried over Na₂SO₄ and concentrated to give the crude product. The residue was purified by column chromatography (SiO₂, Petroleum ether/Ethyl acetate=100/0 to 80/20) to give Compound B-9. LC-MS (m/z): 447.2 [M+H]⁺. ¹H NMR (400 MHz, CHLOROFORM-d) δ ppm 7.41 (br d, J=8.00 Hz, 1H) 7.08-7.22 (m, 1H) 6.44-7.06 (m, 5H) 4.18-4.85 (m, 2H) 3.84 (br d, J=6.63 Hz, 3H) 2.92-3.31 (m, 2H) 2.34-2.79 (m, 1H) 2.15 (br s, 3H) 1.91-2.05 (m, 6H) 1.70 (brs, 6H) 0.89-1.46 (m, 6H) 0.59-0.84 (m, 3H).

### Procedure 22: Synthesis of Compound B-4

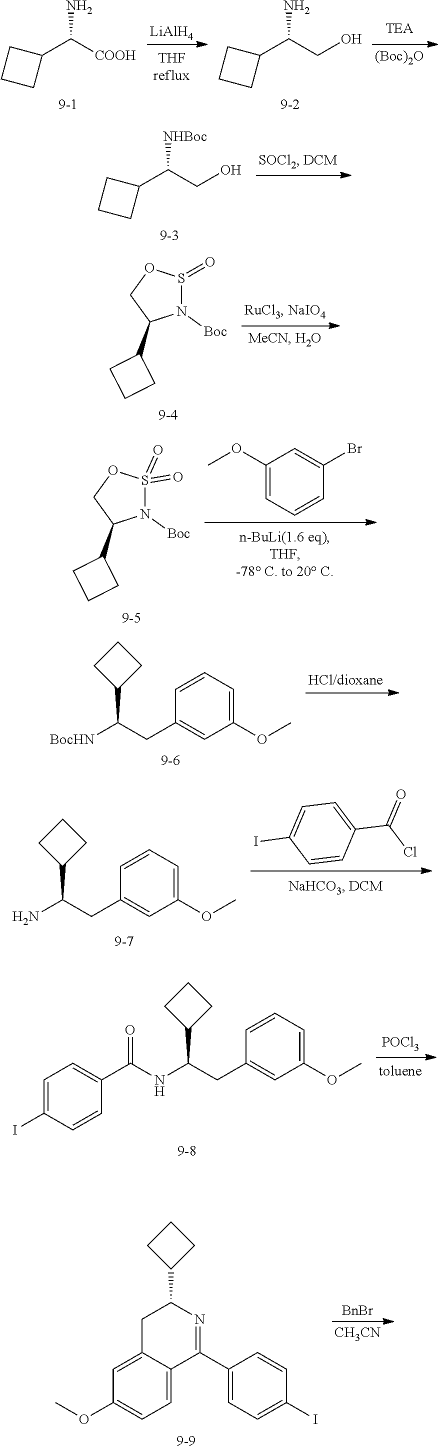

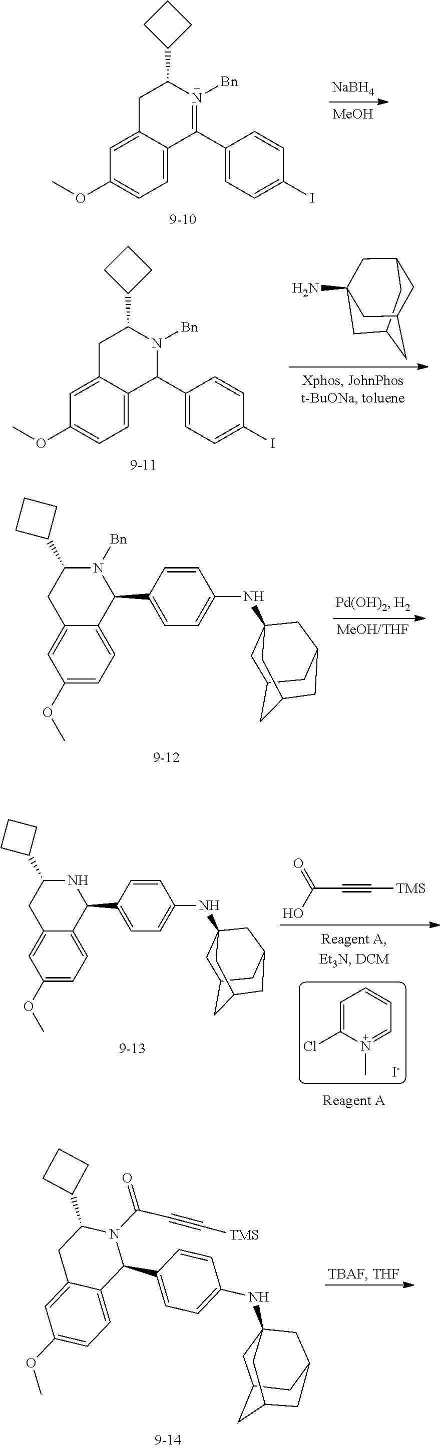

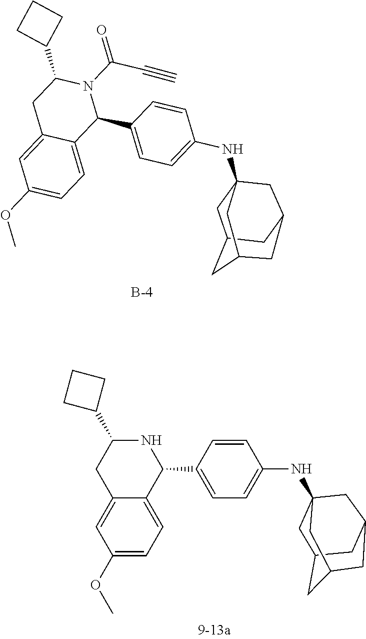

To a solution of LiAlH₄ (1.47 g, 38.71 mmol, 2.5 eq) in THF (20 mL) was added (2S)-2-amino-2-cyclobutyl-acetic acid (2 g, 15.49 mmol, 1 eq) at 0° C. The reaction was stirred at 50° C. for 4 hr to give a yellow suspension. NMR showed the reaction was completed. The reaction mixture was quenched with H₂O (1.47 mL), NaOH (15%, 1.47 mL) and (4.41 mL). The mixture was diluted with THF (30 mL). The mixture was filtered on celite and washed with THF (50 ml). The filtrate was concentrated to give 9-2. Used for next step without further purification. ¹H NMR (CDCl₃, 400 MHz): δ=3.60-3.50 (m, 1H), 3.25-3.15 (m, 1H), 2.80-2.74 (m, 1H), 2.25-1.90 (m, 4H), 1.80-1.66 (m, 3H).

To a solution of 9-2 (1.5 g, 13.02 mmol, 1 eq) in DCM (10 mL) was added Et₃N (1.98 g, 19.54 mmol, 2.72 mL, 1.5 eq) at 0° C. Then Boc₂O (2.84 g, 13.02 mmol, 2.99 mL, 1 eq) in DCM (10 mL) was added dropwise at 0° C. The reaction was allowed to stir at 20° C. for 12 hr to give a yellow solution. TLC (eluting with:PE/EtOAc=2/1) showed the reaction was completed. The reaction was quenched with H₂O (30 mL) and extracted with DCM (30 mL*3). The organic layers were dried over Na₂SO₄ and concentrated to give the crude product. The crude product was purified by flash column (eluting with: PE/EtOAc=0% to 30%) to give 9-3. ¹H NMR (CDCl₃, 400 MHz): δ=4.60 (brs, 1H), 3.82-3.41 (m, 3H), 2.42 (brs, 1H), 2.07-1.80 (m, 6H), 1.47 (s, 9H).

Imidazole (3.71 g, 54.44 mmol, 5.86 eq) was dissolved in DCM (60 mL) and cooled to 0° C. SOCl2 (1.92 g, 16.16 mmol, 1.17 mL, 1.74 eq) was dissolved in DCM (12 mL) was added dropwise and the resulting suspension was allowed to warm to 20° C. Stirring was continued for 1 h at 20° C. and then the mixture was cooled to −78° C. A solution of the title compound from 9-3 (2 g, 9.29 mmol, 1 eq) in DCM (60 mL) was added over a period of 1 h. The resulting mixture was allowed to warm to 20° C. and stirred for 12 hr to give a yellow suspension. TLC eluting with:PE/EtOAc=2/1) showed the reaction was completed. The reaction mixture was filtered and washed with DCM (40 mL). The organic layers were dried over Na₂SO₄ and concentrated to give 9-4. Used for next step without further purification. ¹H NMR (CDCl₃, 400 MHz): δ=4.88-4.02 (m, 3H), 2.80-2.48 (m, 1H), 2.00-1.60 (m, 6H), 1.45 (s, 9H).

To a solution of 9-4 (2.5 g, 9.57 mmol, 1 eq) in CH₃CN (20 mL)/H₂O (10 mL) were added RuCl₃·H₂O (10.78 mg, 47.83 μmol, 0.005 eq)/NaIO₄ (2.25 g, 10.52 mmol, 583.09 uL, 1.1 eq) at 0° C. The reaction was allowed to stir 20° C. for 12 hr to give a yellow solution. TLC (eluting with:PE/EtOAc=3/1) showed the reaction was completed. The reaction mixture was filtered and washed with EtOAc (30 mL*3). The filtrate was washed with H₂O (30 mL). The organic layer was dried over Na₂SO₄ and concentrated to give 9-5. Used for next step without further purification. ¹H NMR (CDCl₃, 400 MHz): δ=4.56-4.50 (m, 1H), 4.25-4.16 (m, 2H), 2.82-2.69 (m, 1H), 2.82-2.69 (m, 1H), 2.00-1.70 (m, 6H), 1.48 (s, 9H)

To a solution of 9-5 (5 g, 18.03 mmol, 1 eq) in THF (50 mL) was added dropwise n-BuLi (2.5 M, 11.54 mL, 1.6 eq) at −78° C. The reaction was stirred at −78° C. for 0.5 hr. Then 1-bromo-3-methoxy-benzene (5.06 g, 27.04 mmol, 3.42 mL, 1.5 eq) in THF (30 mL) was added dropwise at −78° C. The reaction was allowed to stir at 20° C. for 11.5 hr to give a yellow solution. LCMS and TLC (eluting with: PE/EtOAc=5/1) showed the reaction was completed. The reaction mixture was quenched with saturated citric acid (20 mL) and extracted with EtOAc (30 mL*3). The organic layers were dried over Na₂SO₄ and concentrated to give the crude product. The crude product was purified by flash column (eluting with PE/EtOAc=0% to 20%) to give 9-6.

9-6 (1.5 g, 4.91 mmol, 1 eq) was dissolved in HCl/dioxane (4 M, 20 mL, 16.29 eq). The reaction was stirred at 20° C. for 12 hr to give a yellow solution. NMR showed the reaction was completed. The reaction mixture was concentrated to give 9-7. Used for next step without further purification. ¹H NMR (MeOD, 400 MHz): δ ppm 7.31-7.30 (t, J=4.8 Hz, 1H), 6.90-6.82 (m, 3H), 3.82 (s, 3H), 3.44-3.30 (m, 1H), 2.95-2.86 (m, 2H), 2.79-2.74 (m, 1H), 2.01-1.81 (m, 6H).

To a solution of 9-7 (205 mg, 847.96 μmol, 1 eq, HCl) in DCM (10 mL)/H₂O (3 mL) were added NaHCO₃ (71.23 mg, 847.96 μmol, 32.98 uL, 1 eq) and 4-iodobenzoyl chloride (239.51 mg, 898.84 μmol, 1.06 eq). The reaction was stirred at 25° C. for 12 hr to give a yellow solution. LCMS and TLC (eluting with: PE/EtOAc=5/1) showed the reaction was completed. The reaction mixture was diluted with H₂O (20 mL) and extracted with DCM (20 mL*3). The organic layers were dried over Na₂SO₄ and concentrated to give the crude product. The crude product was purified by flash column (eluting with: PE/EtOAc=0% to 30%) to give 9-8. ¹H NMR (CDCl₃, 400 MHz): δ ppm 7.79-7.77 (d, J=6.8 Hz, 1H), 7.42-7.40 (d, J=8.4 Hz, 1H), 7.25-7.18 (m, 1H), 6.80-6.70 (m, 3H), 5.70-6.68 (d, J=9.2 Hz, 3H), 4.45-4.30 (m, 1H), 3.79 (s, 3H), 2.96-2.89 (m, 1H), 2.78-2.70 (m, 1H), 2.45-2.40 (m, 1H), 2.01-1.81 (m, 6H).

To a solution of 9-8 (1.4 g, 3.22 mmol, 1 eq) in toluene (10 mL) was added POCl₃ (4.93 g, 32.16 mmol, 2.99 mL, 10 eq). The reaction was stirred at 140° C. for 1 hr to give a yellow solution. TLC (eluting with: PE/EtOAc=5/1) showed the reaction was completed. The reaction mixture was poured into H₂O (50 ml) and extracted with EtOAc (50 mL*3). The organic layers were dried over Na₂SO₄ and concentrated to give 9-9. Used for next step without further purification.

To a solution of 9-10 (1.34 g, 3.21 mmol, 1 eq) in CH₃CN (20 mL) was added BnBr (1.65 g, 9.63 mmol, 1.14 mL, 3 eq). The reaction was stirred at 85° C. for 12 hr to give a yellow solution. LCMS showed the reaction was completed. The reaction mixture was concentrated to give 9-10. Used for next step without further purification.

To a solution of 9-9 (1.63 g, 3.21 mmol, 1 eq) in MeOH (20 mL) was added NaBH₄ (363.88 mg, 9.62 mmol, 3 eq) at −78° C. to give a solution. TLC (eluting with: PE/EtOAc=3/1) showed the reaction was completed. The reaction mixture was quenched with Sat. NH₄Cl (20 mL) and extracted with EtOAc (20 mL*3). The organic layers were dried over Na₂SO₄ and concentrated to give the crude product. The crude product was purified by flash column (eluting with: PE/EtOAc=0% to 20%) to give 9-11.

To a solution of 9-11 (500 mg, 981.51 μmol, 1 eq) in toluene (8 mL) were added adamantan-1-amine (445.35 mg, 2.94 mmol, 3 eq) and t-BuONa (282.98 mg, 2.94 mmol, 3 eq) under N₂. Then XPhos (93.58 mg, 196.30 μmol, 0.2 eq), JohnPhos (117.15 mg, 392.60 μmol, 0.4 eq) and Pd₂(dba)₃ (89.88 mg, 98.15 μmol, 0.1 eq) were added under N₂. The reaction was stirred at 125° C. for 12 hr to give a black suspension. LCMS and TLC (eluting with: PE/EtOAc=5/1) showed the reaction was completed. The reaction mixture was concentrated to give the crude product. The crude product was purified by flash column (eluting with: PE/EtOAc=0% to 30%) to give 9-12. ¹H NMR (CDCl₃, 400 MHz): δ ppm 7.32-7.25 (m, 4H), 7.24-7.18 (m, 1H), 6.86-6.60 (m, 7H), 4.58-4.47 (m, 1H), 3.76-3.50 (m, 5H), 3.34-3.20 (m, 1H), 2.90-2.70 (m, 1H), 2.64-2.40 (m, 3H), 2.01-1.85 (m, 5H), 1.78-1.60 (m, 7H), 1.54-1.50 (m, 8H)

To a solution of 9-12 (320 mg, 600.65 μmol, 1 eq) in EtOH (20 mL) were added Pd/C (100 mg, 50% purity) and conc. HCl (110.00 mg, 1.32 mmol, 0.1 mL, 2.19 eq) under N₂. The suspension was degassed under vacuum and purged with H₂ several times. The mixture was stirred under H₂ (1.21 mg, 600.65 μmol, 1 eq) (15 psi) at 25° C. for 2 hours to give a yellow solution to give a black suspension. LCMS and TLC (eluting with: PE/EtOAc=1/1) showed the reaction was completed. The reaction mixture was filtered and washed with MeOH (20 mL). The filtrate was concentrated to give the crude product. The crude product was purified by flash column (eluting with: PE/EtOAc=0% to 50%) to give 9-13 and 9-13a. ¹H NMR (CDCl₃, 400 MHz): δ ppm 6.86-6.74 (m, 3H), 6.66-6.56 (m, 5H), 5.02 (s, 1H), 3.76-3.72 (s, 3H), 2.83-2.70 (m, 2H), 2.45-2.23 (m, 2H), 2.05-1.94 (m, 3H), 1.92-1.85 (m, 3H), 1.80-1.70 (m, 15H)

To a solution of 3-trimethylsilylprop-2-ynoic acid (37.59 mg, 264.33 μmol, 0.9 eq) in DCM (5 mL) was added 2-chloro-1-methylpyridin-1-ium iodide (67.53 mg, 264.33 μmol, 0.9 eq). The reaction was stirred at 25° C. for 0.5 hr. Then the mixture was added dropwise the solution of 9-13 (130 mg, 293.70 μmol, 1 eq) and Et₃N (29.72 mg, 293.70 μmol, 40.88 uL, 1 eq) in DCM (5 mL) at 0° C. The reaction was stirred at 0° C. for 0.5 hr to give a yellow solution. TLC (eluting with: PE/EtOAc=2/1) showed the reaction was completed. The reaction mixture was quenched with H₂O (0.5N, 10 mL) and extracted with DCM (20 mL*3). The organic layers were washed with Sat·NaHCO₃ (15 mL). The organic layers were dried over Na₂SO₄ and concentrated to give the crude product. The crude product was purified by flash column (eluting with: PE/EtOAc=0% to 30%) to give 9-14. LC-MS (m/z): 567.3[M+H]⁺. ¹H NMR (400 MHz, CDCl₃) δ=7.19-7.02 (m, 1H), 6.88-6.84 (m, 2H), 6.71-6.48 (m, 4H), 6.15-5.92 (m, 1H), 4.65-4.48 (m, 1H), 3.70-3.67 (m, 3H), 3.14-3.05 (m, 2H), 2.82-2.52 (m, 1H), 2.15-1.80 (m, 5H), 1.71-1.65 (m, 8H), 1.63-1.58 (m, 8H), 0.18-0.08 (m, 9H).

To a solution of 9-14 (20 mg, 35.28 μmol, 1 eq) in THF (10 mL) was added TBAF (1 M, 35.28 uL, 1 eq). The reaction was stirred at −78° C. The reaction was stirred at −78° C. for 0.5 hr to give a yellow solution. TLC (eluting with: PE/EtOAc=2/1) showed the reaction was completed. The reaction mixture was quenched with H₂O (10 mL) and extracted with EtOAc (30 mL*3). The organic layers were dried over Na₂SO₄ and concentrated to give the crude product. The crude product was purified by flash column (eluting with: PE/EtOAc=0% to 30%) to give B-4. LC-MS (m/z): 495.2[M+H]⁺

¹H NMR (400 MHz, CDCl₃) δ=7.25-7.13 (m, 1H), 7.00-6.92 (m, 2H), 6.81-6.75 (m, 1H), 6.68-6.60 (m, 3H), 6.70-6.02 (m, 1H), 4.75-4.45 (m, 1H), 3.83-3.78 (m, 3H), 3.25-2.65 (m, 3H), 2.30-2.10 (m, 1H), 2.05-1.95 (m, 3H), 1.90-1.85 (m, 1H), 1.68-1.60 (m, 17H).

### Procedure 23: Synthesis of Compound B-8

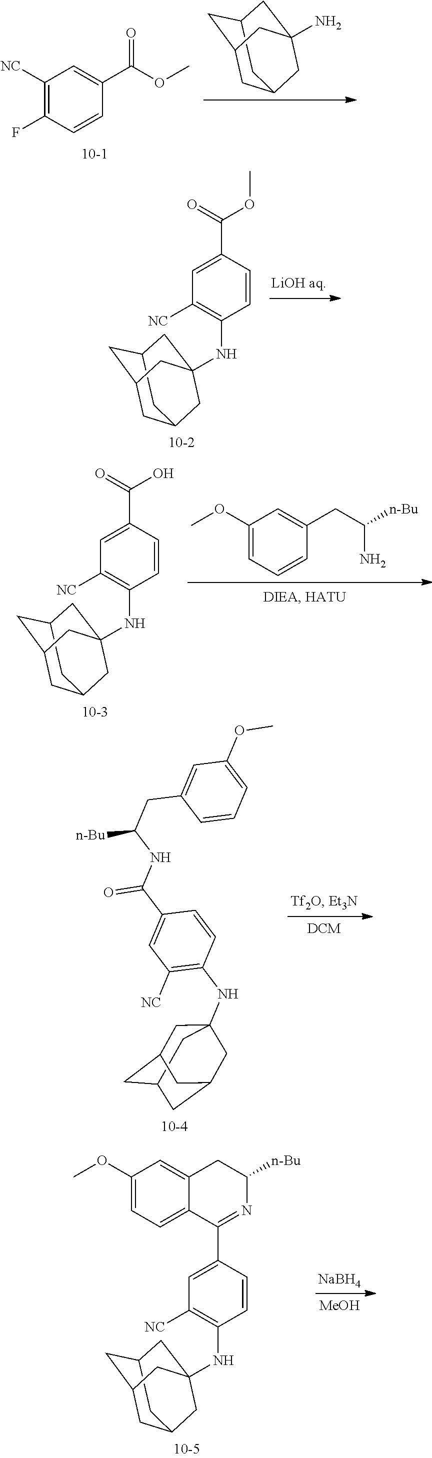

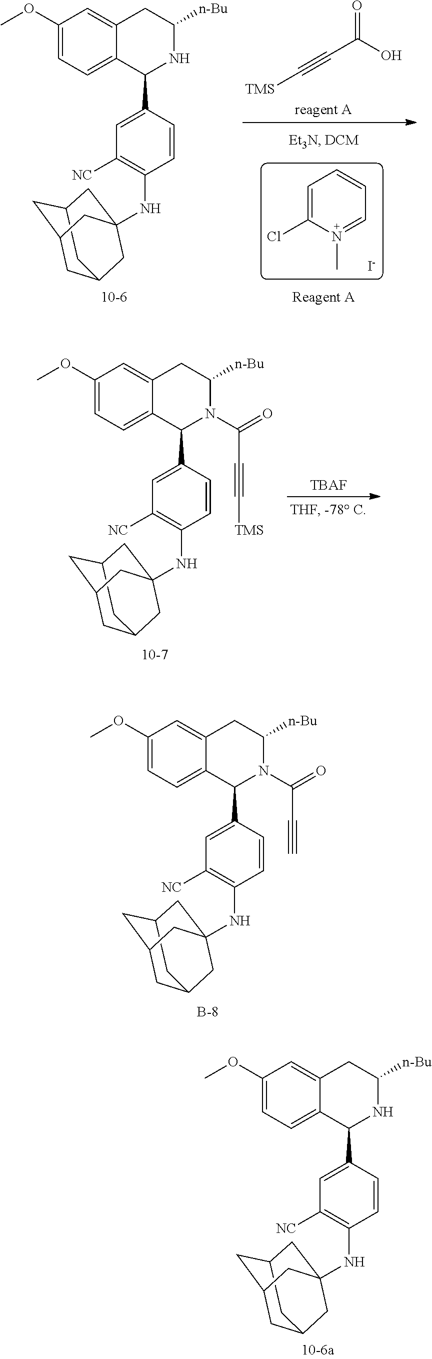

To a solution of 4-(1-adamantylamino)-3-cyano-benzoic acid (500 mg, 1.69 mmol, 1 eq) in DMF (10 mL) were added DIEA (436.10 mg, 3.37 mmol, 587.73 uL, 2 eq) and (2S)-1-(3-methoxyphenyl)hexan-2-amine (349.76 mg, 1.69 mmol, 1 eq) and HATU (673.57 mg, 1.77 mmol, 1.05 eq) at 0° C. The reaction was allowed to stir at 25° C. for 12 hr to give a yellow solution. LCMS and TLC (eluting with: PE/EtOAc=3/1) showed the reaction was completed. The reaction mixture was quenched with H₂O (30 mL) and extracted with MBTE (30 ML*3). The organic layers were dried over Na₂SO₄ concentrated to give the crude product. The crude product was purified by flash column (eluting with: PE/EtOAc=0% to 30%) to give 10-4. ¹H NMR (400 MHz, CDCl₃) δ=7.63-7.58 (m, 1H), 7.17-7.15 (t, J=8.0 Hz, 1H), 6.95-6.90 (m, 1H), 6.73-6.64 (m, 3H), 5.58-5.54 (m, 1H), 4.68 (s, 1H), 4.33-4.25 (m, 1H), 3.71 (s, 3H), 2.85-2.70 (m, 2H), 2.11 (s, 3H), 1.94 (s, 6H), 1.68-1.60 (m, 6H), 1.45-1.20 (m, 6H), 0.82-0.80 (t, J=6.8 Hz, 3H).

To solution of 10-4 (500 mg, 1.03 mmol, 1 eq) in DCM (10 mL) were added 2-chloropyridine (350.69 mg, 3.09 mmol, 292.24 uL, 3 eq) and Tf₂O (871.41 mg, 3.09 mmol, 509.60 uL, 3 eq) at −78° C. The reaction was allowed to stir at 25° C. for 1 h to give a yellow solution. LCMS showed the reaction was completed. The reaction mixture was quenched with Sat. NaHCO₃ (20 mL) and extracted with DCM (30 mL*3). The organic layers were dried over Na₂SO₄ and concentrated to give the crude product. The crude product was purified by flash column (eluting with: PE/EtOAc=0% to 30%) to give 10-5. ¹H NMR (400 MHz, CDCl₃) δ=7.60-7.52 (m, 1H), 7.19-7.14 (m, 1H), 7.00-6.92 (m, 1H), 6.73-6.64 (m, 3H), 4.52 (brs, 1H), 3.79 (s, 3H), 3.33 (brs, 1H), 2.72-2.60 (m, 1H), 2.49-2.40 (m, 1H), 2.10 (s, 3H), 1.96 (s, 6H), 1.68-1.60 (m, 6H), 1.45-1.20 (m, 6H), 0.91-0.86 (m, 3H).

To a solution of 10-5 (320 mg, 684.28 μmol, 1 eq) in MeOH (10 mL) was added NaBH₄ (129.44 mg, 3.42 mmol, 5 eq). The reaction was stirred at 25° C. for 0.5 hr to give a yellow solution. TLC (eluting with: PE/EtOAc=2/1) showed the reaction was completed. The reaction mixture was quenched with Sat·NH₄Cl (10 mL) and extracted with EtOAc (20 mL*3). The organic layers were dried over Na₂SO₄ and concentrated to give the crude product. The crude product was purified by flash column (eluting with: PE/EtOAc=1/1) to give 10-6 and 10-6a. ¹H NMR (400 MHz, CDCl₃) δ=7.19-7.10 (m, 1H), 7.00-6.87 (m, 1H), 6.84-6.80 (m, 1H), 6.74-6.68 (m, 1H), 6.64-6.60 (m, 1H), 5.00 (brs, 1H), 4.29 (s, 1H), 3.33 (brs, 1H), 3.76 (s, 3H), 2.84-2.70 (m, 2H), 2.52-2.45 (m, 1H), 2.08 (s, 3H), 1.90 (s, 6H), 1.68-1.60 (m, 6H), 1.45-1.20 (m, 6H), 0.82-0.80 (t, J=7.2 Hz, 3H).

To a solution of 3-trimethylsilylprop-2-ynoic acid (21.80 mg, 153.30 μmol, 0.9 eq) in DCM (5 mL) was added 2-chloro-1,6-dimethylpyridin-1-ium (21.86 mg, 153.30 μmol, 0.9 eq). The reaction was stirred at 25° C. for 0.5 hr. Then the mixture was added dropwise the solution of 10-6 (80 mg, 170.34 μmol, 1 eq) and Et₃N (17.24 mg, 170.34 μmol, 23.71 uL, 1 eq) in DCM (5 mL) at 0° C. The reaction stirred at 0° C. for 0.5 hr to give a yellow solution. TLC (eluting with: PE/EtOAc=2/1) showed the reaction was completed. The reaction mixture was diluted with HCl (1N, 15 mL) and extracted with DCM (20 mL*3). The organic layers were dried over Na₂SO₄ and concentrated to give the crude product. The crude product was purified by flash column (eluting with: PE/EtOAc=0% to 30%) to give 10-7. LC-MS (m/z): 594.2[M+H]⁺. ¹H NMR (400 MHz, DMSO-d6) δ=7.08-7.05 (m, 2H), 6.92-6.82 (m, 1H), 6.80-6.69 (m, 2H), 6.62-6.58 (m, 1H), 6.05-6.00 (m, 2H), 4.48-4.40 (m, 1H), 4.22-4.18 (m, 1H), 3.71 (s, 3H), 3.00-2.60 (m, 2H), 2.00 (s, 3H), 1.86-1.76 (m, 6H), 1.68-1.60 (m, 6H), 1.45-1.05 (m, 6H), 0.78-0.72 (m, 3H), 0.18-0.02 (m, 9H).

To a solution of 10-7 (30 mg, 50.52 μmol, 1 eq) in THF (5 mL) was added TBAF (1 M, 50.52 uL, 1 eq) −78° C. The reaction was stirred at −78° C. for 0.5 hr to give a yellow solution. TLC (eluting with: PE/EtOAc=3/1) showed the reaction was completed. The reaction mixture was quenched with H₂O (15 mL) and extracted with EtOAc (30 mL*3). The organic layers were dried over Na₂SO₄ and concentrated to give the crude product. The crude product was purified by flash column (eluting with: PE/EtOAc=0% to 30%) to give B-8. LC-MS (m/z): 522.3[M+H]⁺. ¹H NMR (400 MHz, DMSO-d6) δ=7.08-7.05 (m, 2H), 6.92-6.82 (m, 1H), 6.80-6.69 (m, 2H), 6.62-6.58 (m, 1H), 6.05-6.00 (m, 2H), 4.48-4.40 (m, 1H), 4.22-4.18 (m, 1H), 3.71 (s, 3H), 3.00-2.60 (m, 2H), 2.00 (s, 3H), 1.86-1.76 (m, 6H), 1.68-1.60 (m, 6H), 1.45-1.05 (m, 6H), 0.78-0.72 (m, 3H), 0.18-0.02 (m, 9H).

### Procedure 24: Synthesis of Compound B-14

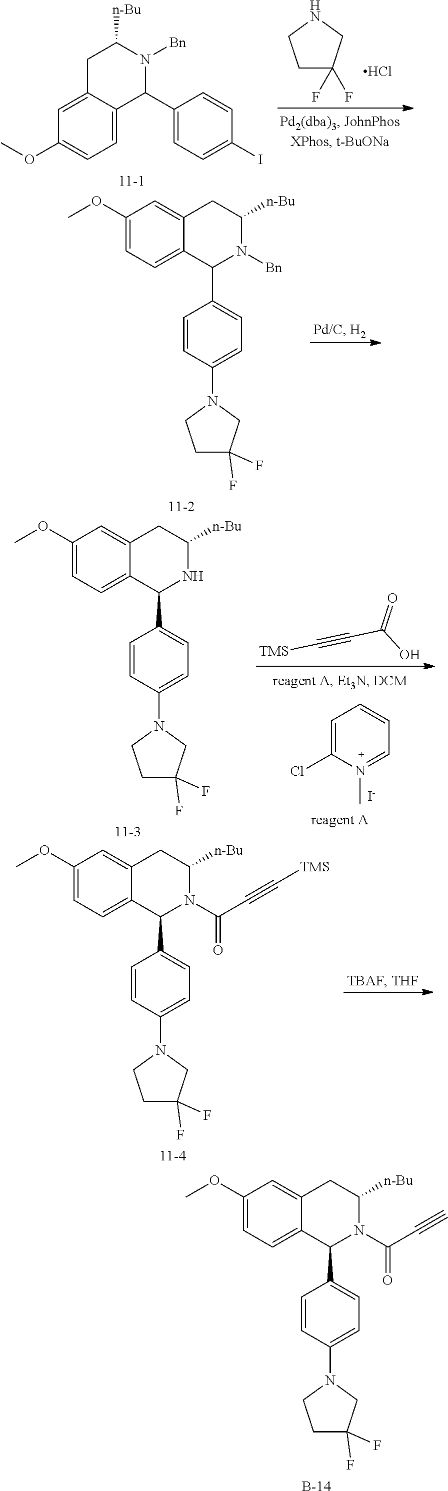

To a solution of 11-1 in toluene (5 mL) were added 3,3-difluoropyrrolidine; hydrochloride (μmol. 60 mg, 586.58 μmol, 3 eq, HCl), t-BuONa (93.95 mg, 977.64 μmol, 5 eq), XPhos (18.64 mg, 39.11 μmol, 0.2 eq), JohnPhos (11.67 mg, 39.11 μmol, 0.2 eq) and Pd₂(dba)₃ (17.90 mg, 19.55 μmol, 0.1 eq). The reaction was stirred at 125° C. under N₂ for 12 hr to give black suspension. TLC and LCMS show the reaction was completed. The reaction mixture was added DCM (50 ml) and concentrated to give crude product. The crude product was purified by flash column (SiO₂, eluting with:PE/EtOAc=0-10%) to give 11-2. ¹H NMR (400 MHz, CDCl₃) δ ppm 7.26-7.19 (m, 1H) 6.98-6.90 (m, 2H) 6.85-6.75 (m, 1H) 6.70-6.66 (m, 1H) 6.46-6.35 (m, 2H) 6.24-6.19 (m, 1H) 4.61-4.48 (m, 1H) 3.82 (br s, 1H) 3.81-3.80 (m, 3H) 3.78-3.69 (m, 1H) 3.16-2.85 (m, 1H) 2.74-2.67 (m, 1H) 2.42-2.31 (m, 2H) 1.84-1.70 (m, 4H) 1.32-1.16 (m, 6H) 0.88-0.80 (m, 3H) 0.29-0.07 (m, 9H)

To a solution of 11-2 (180 mg, 366.88 μmol, 1 eq) in EtOH (10 mL) was added Pd/C (100 mg, 50% purity, 1.00 eq) and Conc. HCl (110.00 mg, 1.32 mmol, 0.1 mL, 3.59 eq) under N₂. The suspension was degassed under vacuum and purged with H₂ several times. The mixture was stirred under H₂ (15 Psi) at 25° C. for 12 hours to give a black suspension. LCMS showed the reaction was completed. The reaction mixture was filtered on celite and washed with MeOH (30 mL). The filtrate was concentrated to give the crude product. The crude product was purified by flash column (eluting with: PE/EtOAc=0% to 40%) to give 11-3. ¹H NMR (400 MHz, CDCl₃) δ ppm 7.02-6.94 (m, 2H), 6.77-6.74 (m, 1H), 6.62-6.53 (m, 2H), 6.43-6.40 (m, 2H), 5.09 (s, 1H) 3.73 (s, 3H), 3.63-3.55 (m, 2H), 3.45-3.42 (m, 2H), 2.90-2.78 (m, 2H), 2.44-2.36 (m, 3H), 1.44-1.20 (m, 1H), 0.81-0.79 (t, J=6.8 Hz, 3H).

To a solution of 3-trimethylsilylprop-2-ynoic acid (31.96 mg, 224.72 μmol, 1 eq) in DCM (5 mL) were added 2-chloro-1-methylpyridin-1-ium iodide (57.41 mg, 224.72 μmol, 1 eq). The reaction was stirred at 20° C. for 0.5 hr. Then 11-3 and Et₃N (22.74 mg, 224.72 μmol, 31.28 uL, 1 eq) was added dropwise at 0° C. The reaction was stirred at 0° C. for 0.5 hr to give a solution. TLC (eluting with: PE/EtOAc=2/1) showed the reaction was completed. The reaction mixture was quenched with H₂O (20 mL) and extracted with DCM (30 mL*2). The organic layers were washed with HCl (1N, 10 mL). The organic layer was separated. The organic layer was washed Sat·NaHCO₃ (15 mL). The organic layer was separated. The organic layer was dried over Na₂SO₄ and concentrated to give the crude product. The crude product was purified by flash column (SiO₂, eluting with: PE/EtOAc=0% to 40%) to give 11-4. LC-MS (m/z): 525.2 [M+H]⁺. ¹H NMR (400 MHz, CDCl₃) δ ppm 7.26-7.17 (m, 1H) 7.11-6.94 (m, 2H) 6.86-6.75 (m, 1H) 6.73-6.62 (m, 1H) 6.50-6.37 (m, 2H) 6.33-6.15 (m, 1H) 4.68-4.40 (m, 1H) 3.86-3.75 (m, 3H) 3.68-3.54 (m, 2H) 3.52-3.38 (m, 2H) 3.20-2.80 (m, 1H) 2.78-2.63 (m, 1H) 2.55-2.35 (m, 2H) 1.31-1.13 (m, 6H) 0.89-0.80 (m, 3H) 0.06-0.33 (m, 9H).

To a solution of 11-4 in THF (5 mL) was stirred at −78° C. for 10 min, Then TBAF (1 M, 41.93 uL, 1.1 eq) was added. The reaction was stirred at −78° C. for 10 min to give yellow suspension. TLC (eluting with: PE/EtOAc=2/1) show the reaction was completed. The reaction mixture was quenched with H₂O (20 mL) and extracted with DCM (30 mL*2). The organic layer was dried over Na₂SO₄ and concentrated to give the crude product. The crude product was purified by flash column (SiO₂, eluting with: PE/EtOAc=0% to 30%) to give B-14. LC-MS (m/z): 453.2 [M+H]⁺. ¹H NMR (400 MHz, CDCl₃) δ ppm 7.35-7.30 (m, 1H) 7.23-7.18 (m, 1H) 7.11-6.99 (m, 2H) 6.86-6.66 (m, 2H) 6.50-6.35 (m, 2H) 6.33-6.14 (m, 1H) 4.71-4.43 (m, 1H) 3.86-3.76 (m, 3H) 3.67-3.53 (m, 2H) 3.49-3.40 (m, 2H) 3.19-3.09 (m, 1H) 2.97-2.60 (m, 2H) 2.53-2.32 (m, 2H) 1.38-1.11 (m, 6H) 0.88-0.82 (m, 3H).

### Procedure 25: Synthesis of Compound B-46 and Compound B-52

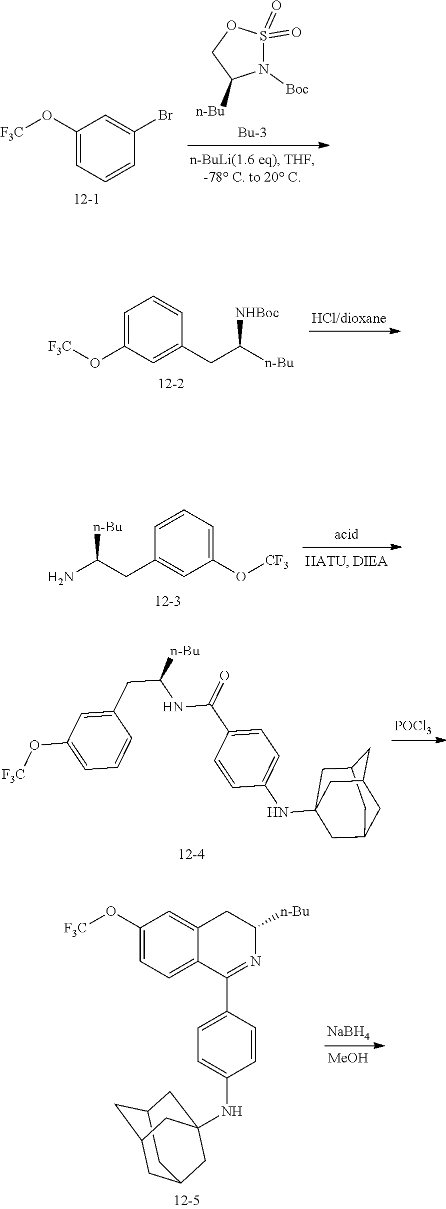

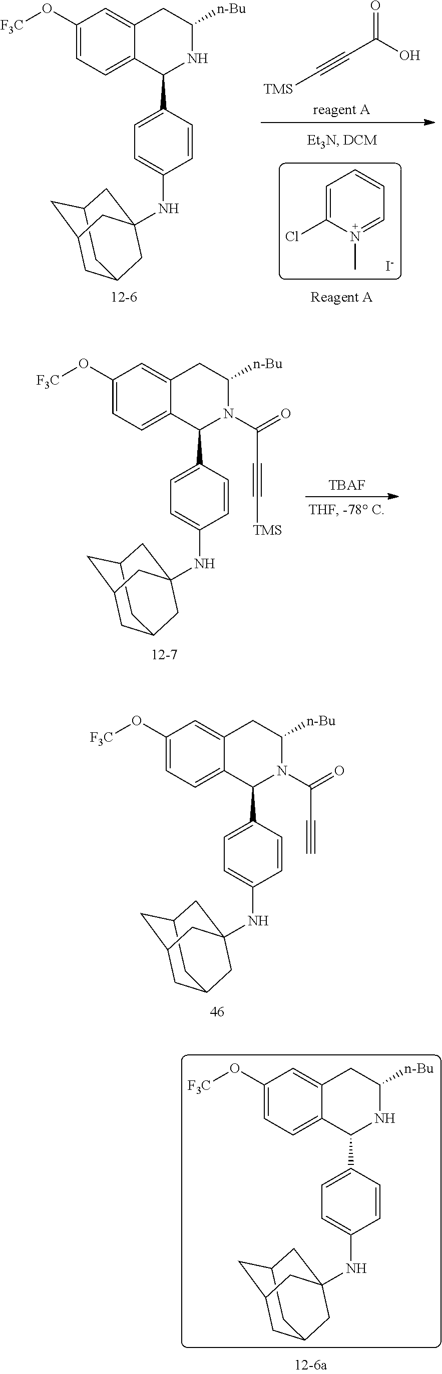

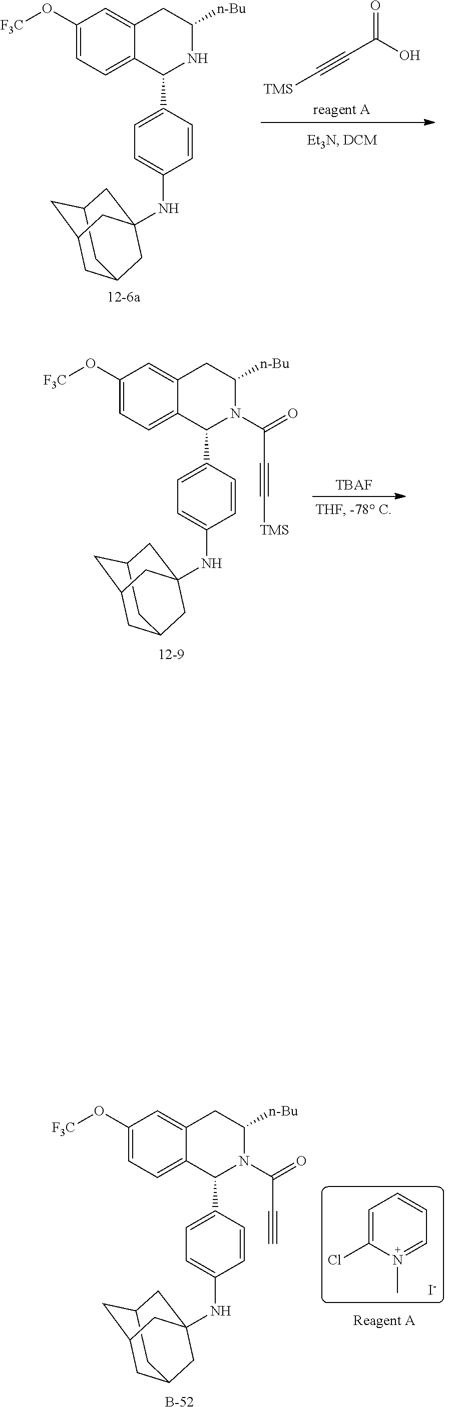

To a solution of 12-1 (6.5 g, 26.97 mmol, 4.01 mL, 1.5 eq) in THF (50 mL) was added dropwise n-BuLi (2.5 M, 11.51 mL, 1.6 eq) at −78° C. The reaction was stirred at −78° C. for 0.5 hr. Then Bu-3 (5.02 g, 17.98 mmol, 1 eq) in THF (30 mL) was added dropwise at −78° C. The reaction was allowed to stir at 20° C. for 12 hr to give a yellow solution. LCMS and TLC (eluting with: PE/EtOAc=6/1) showed the reaction was completed. The reaction mixture was quenched with Sat. citric acid (20 mL) and extracted with EtOAc (30 mL*3). The organic layers were dried over Na₂SO₄ and concentrated to give the crude product. The crude product was purified by flash column (eluting with PE/EtOAc=0% to 15%) to give 12-2. ¹H NMR (400 MHz, CDCl₃) δ ppm 7.26-7.20 (m, 1H), 7.18-7.08 (m, 1H), 7.02 (br dd, J=19.95, 7.57 Hz, 1H), 4.31-4.14 (m, 1H), 2.78-2.58 (m, 1H), 1.38-1.30 (m, 8H), 1.29 (br s, 2H), 1.27-1.23 (m, 1H), 1.21 (br dd, J=5.44, 2.06 Hz, 1H), 0.83-0.77 (m, 3H).

To a solution of 12-2 (1.6 g, 4.43 mmol, 1 eq) was dissolved in HCl/dioxane (4 M, 1.11 mL, 1 eq) at 0° C. The reaction was allowed to stir at 25° C. for 12 hr to give a colorless oil. LCMS showed the reaction was completed. The reaction mixture was concentrated directly to give 12-3. The product was used for next further without purification. ¹H NMR (400 MHz, MeOD) δ ppm 7.53-7.47 (m, 1H), 7.47 (br s, 1H), 7.36-7.29 (m, 1H), 7.25 (br s, 2H), 3.50 (quin, J=6.69 Hz, 1H), 3.01 (d, J=7.13 Hz, 2H), 1.70-1.58 (m, 2H), 1.42-1.32 (m, 4H), 0.96-0.91 (m, 1H), 0.98-0.90 (m, 3H).

To a solution of 4-(1-adamantylamino)benzoic acid (1.04 g, 3.83 mmol, 1 eq) in DMF (20 mL) was added DIEA (989.30 mg, 7.65 mmol, 1.33 mL, 2 eq), 12-3 (1 g, 3.83 mmol, 1 eq) and HATU (1.53 g, 4.02 mmol, 1.05 eq) at 0° C. The reaction was allowed to stir at 0 to 25° C. for 12 hr to black brown liquid. LCMS and TLC (eluting with: PE/EtOAc=3/1) showed the reaction was completed. The reaction mixture was quenched with H₂O (100 mL) and extracted with MBTE (30 ML*3). The organic layers were dried over Na₂SO₄ concentrated to give the crude product. The crude product was purified by flash column (eluting with: PE/EtOAc=0% to 20%) to give 12-4. ¹H NMR (400 MHz, CDCl₃) δ ppm 7.45-7.39 (m, 2H), 7.27-7.21 (m, 1H), 7.09 (d, J=7.63 Hz, 1H), 7.03-6.97 (m, 2H), 6.65-6.59 (m, 2H), 4.33-4.23 (m, 1H), 2.86-2.77 (m, 2H), 2.06 (br s, 3H), 1.91-1.84 (m, 7H), 1.63 (br s, 6H), 1.31-1.27 (m, 2H), 1.19 (t, J=7.13 Hz, 4H), 0.81-0.76 (m, 4H).

To solution of 12-4 (620 mg, 1.20 mmol, 1 eq) in DCM (10 mL) were added 2-chloropyridine (410.41 mg, 3.61 mmol, 342.00 uL, 3 eq) and Tf₂O (1.02 g, 3.61 mmol, 596.34 uL, 3 eq) at −78° C. The reaction was allowed to stir at 25° C. for 12 h to give a yellow solution. LCMS and TLC (eluting with: PE/EtOAc=3/1) showed the reaction was completed. The reaction mixture was quenched with Sat·NaHCO₃ (20 mL) and extracted with DCM (30 mL*3). The organic layers were dried over Na₂SO₄ and concentrated to give the crude product. The crude product was purified by flash column (eluting with: PE/EtOAc=0% to 30%) to give 12-5. ¹H NMR (400 MHz, CDCl₃) δ ppm 7.53-7.48 (m, 1H), 7.48-7.40 (m, 3H), 7.13-7.05 (m, 3H), 3.55-3.38 (m, 1H), 2.98-2.89 (m, 1H), 2.67-2.53 (m, 1H), 2.00-1.92 (m, 9H), 1.75-1.70 (m, 8H), 1.46-1.35 (m, 4H), 0.98-0.93 (m, 3H).

To a solution of 12-5 (170 mg, 342.32 μmol, 1 eq) in MeOH (5 mL) was added NaBH₄ (64.75 mg, 1.71 mmol, 5 eq). The reaction was stirred at 25° C. for 0.5 hr to give a yellow solution. TLC (eluting with: PE/EtOAc=2/1) showed the reaction was completed. The reaction was quenched with Sat·NH₄Cl (10 mL) and extracted with EtOAc (20 mL*3). The organic layers were dried over Na₂SO₄ and concentrated to give the crude product. The crude product was purified by flash column (eluting with: PE/EtOAc=0% to 50%) to give 12-6 and 12-6a. cis¹H NMR (400 MHz, CDCl₃) δ ppm 7.12-7.06 (m, 2H), 6.96-6.84 (m, 2H), 6.79-6.74 (m, 3H), 4.95 (s, 1H), 3.08-2.82 (m, 3H), 2.13 (s, 3H), 1.90 (s, 6H), 1.72-1.66 (m, 6H), 1.44-1.35 (m, 6H), 0.96-0.92 (m, 3H).

To a solution of 3-trimethylsilylprop-2-ynoic acid (7.96 mg, 55.95 μmol, 0.9 eq) in DCM (3 mL) was added 2-chloro-1-methyl-pyridin-1-ium; iodide (14.30 mg, 55.95 μmol, 0.9 eq). The reaction was stirred at 25° C. for 0.5 hr. Then the mixture was added dropwise the solution of 12-6 (31.00 mg, 62.17 μmol, 1 eq) and Et₃N (6.29 mg, 62.17 μmol, 8.65 uL, 1 eq) in DCM (3 mL) at 0° C. LCMS showed the reaction was completed. The reaction mixture was quenched with H₂O (0.5N, 10 mL) and extracted with DCM (20 mL*3). The organic layers were washed with Sat·NaHCO₃ (15 mL). The organic layers were dried over Na₂SO₄ and concentrated to give the crude product. The crude product was purified by flash column (eluting with: PE/EtOAc=0% to 30%) to give 12-7.

To a solution of 3-trimethylsilylprop-2-ynoic acid (26.96 mg, 189.52 μmol, 0.9 eq) in DCM (5 mL) was added 2-chloro-1-methylpyridin-1-ium; iodide (48.42 mg, 189.52 μmol, 0.9 eq). The reaction was stirred at 25° C. for 0.5 hr. Then the mixture was added dropwise the solution of 12-6a (105.00 mg, 210.58 μmol, 1 eq) and Et₃N (21.31 mg, 210.58 μmol, 29.31 uL, 1 eq) in DCM (5 mL) at 0° C. The reaction was stirred at 0° C. for 0.5 hr to give a yellow solution. LCMS showed the reaction was completed. The reaction mixture was quenched with H₂O (0.5N, 10 mL) and extracted with DCM (20 mL*3). The organic layers were washed with Sat·NaHCO₃ (15 mL). The organic layers were dried over Na₂SO₄ and concentrated to give the crude product. The crude product was purified by flash column (eluting with: PE/EtOAc=0% to 30%) to give 12-9.

To a solution of 12-7 (16 mg, 25.69 μmol, 1 eq) in THF (6 mL) was added TBAF (1 M, 25.69 uL, 1 eq). The reaction was stirred at −78° C. The reaction was stirred at −78° C. for 0.5 hr to give a yellow solution. TLC (eluting with: PE/EtOAc=3/1) showed the reaction was completed. The reaction mixture was quenched with H₂O (10 mL) and extracted with EtOAc (30 mL*3). The organic layers were dried over Na₂SO₄ and concentrated to give the crude product. The crude product was purified by flash column (eluting with: PE/EtOAc=0% to 50%) to give 12-8 (B-46). LC-MS (m/z): 551.5[M+H]⁺. ¹H NMR (400 MHz, CDCl₃) δ ppm 7.44-7.30 (m, 1H), 7.20-7.06 (m, 1H), 7.04-6.98 (m, 1H), 6.95-6.83 (m, 2H), 6.72-6.51 (m, 2H), 6.39-6.07 (m, 1H), 4.74-4.19 (m, 1H), 3.23-2.62 (m, 3H), 2.12-2.02 (m, 3H), 1.87-1.80 (m, 6H), 1.75-1.65 (m, 6H), 1.35-1.07 (m, 6H), 0.86-0.78 (m, 3H).

To a solution of 12-9 (32.00 mg, 51.38 μmol, 1 eq) in THF (12 mL) was added TBAF (1 M, 51.38 uL, 1 eq). The reaction was stirred at −78° C. The reaction was stirred at −7° C. for 0.5 hr to give a yellow solution. TLC (eluting with: PE/EtOAc=3/1) showed the reaction was completed. The reaction mixture was quenched with H₂O (10 mL) and extracted with EtOAc (30 mL*3). The organic layers were dried over Na₂SO₄ and concentrated to give the crude product. The crude product was purified by flash column (eluting with: PE/EtOAc=0% to 30%) to give B-52. LC-MS (m/z): 551.5[M+H]⁺. ¹H NMR (400 MHz, CDCl₃) δ ppm 7.18-6.90 (m, 3H), 6.81 (d, J=8.25 Hz, 1H), 6.77-6.56 (m, 3H), 4.66-4.14 (m, 1H), 3.16-2.85 (m, 2H), 2.71-2.36 (m, 1H), 2.03 (br d, J=1.13 Hz, 3H), 1.79 (dd, J=6.63, 2.38 Hz, 7H), 1.63-1.53 (m, 6H), 1.29-0.79 (m, 6H), 0.77-0.60 (m, 3H).

### Procedure 26: Compound B-50

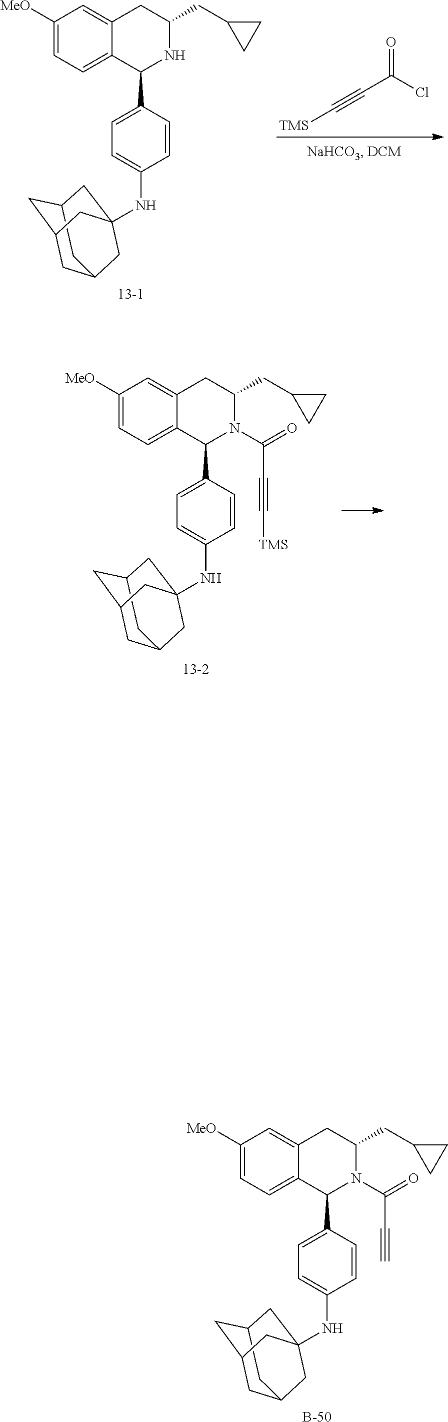

To a solution of 13-1 (50 mg, 112.96 μmol, 1 eq) in DCM (1 mL) were added NaHCO₃ (75.92 mg, 903.68 μmol, 35.15 uL, 8 eq) and 3-trimethylsilylprop-2-ynoyl chloride (0.2 M, 1.13 mL, 2 eq). The resulting mixture was stirred at 20° C. for 12 hr to give yellow suspension. LCMS and TLC (PE/EtOAc=3:1) showed the reaction were completed. The solution was washed H₂O (10 mL) and extracted with DCM (10 mL*3). The organic layer was dried over Na₂SO₄, filtrated and concentrated. The crude product was purified by flash column (SiO₂, eluting with: PE/EtOAc=0% to 30%) to give 13-2 and 50. LC-MS (m/z): 367.3 [M+H]⁺. ¹H NMR (400 MHz, CDCl₃) δ ppm 7.30 (s, 1H) 7.21 (d, J=8.53 Hz, 1H) 6.94 (dd, J=8.53, 3.26 Hz, 2H) 6.79 (td, J=8.03, 2.51 Hz, 1H) 6.73-6.61 (m, 3H) 6.20 (d, J=9.03 Hz, 1H) 4.78-4.64 (m, 1H) 3.81 (s, 3H) 3.23-2.92 (m, 2H) 2.07 (br s, 3H) 1.81 (br s, 6H) 1.57-0.77 (m, 9H) 0.66-0.54 (m, 1H) 0.53-0.34 (m, 2H) 0.28-0.08 (m, 9H) 0.08-−0.05 (m, 2H). LC-MS (m/z): 495.3 [M+H]⁺. ¹H NMR (400 MHz, CDCl₃) δ ppm 7.29 (d, J=8.28 Hz, 1H) 7.21 (d, J=8.53 Hz, 1H) 6.91 (dd, J=15.18, 8.41 Hz, 2H) 6.80 (ddd, J=15.18, 8.28, 2.64 Hz, 1H) 6.74-6.70 (m, 1H) 6.64 (dd, J=11.04, 8.53 Hz, 2H) 6.26-6.15 (m, 1H) 4.82-4.56 (m, 1H) 3.81 (d, J=5.77 Hz, 3H) 3.22-3.10 (m, 1H) 3.03-2.81 (m, 2H) 2.13-2.00 (m, 3H) 1.83 (dd, J=11.42, 2.38 Hz, 6H) 1.59-1.41 (m, 6H) 1.35-0.79 (m, 3H) 0.72-0.55 (m, 1H) 0.53-0.32 (m, 2H) 0.16-−0.05 (m, 2H).

### Procedure 27: Compound B-51

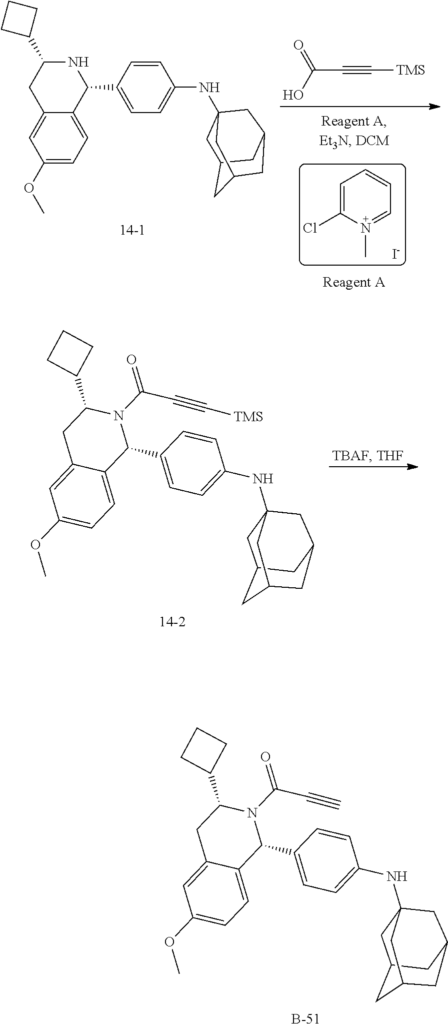

To a solution of 3-trimethylsilylprop-2-ynoic acid (22.49 mg, 158.14 μmol, 1 eq) in DCM (3 mL) was added 2-chloro-1-methyl-pyridin-1-ium; iodide (40.40 mg, 158.14 μmol, 1 eq). The mixture was stirred at 25° C. for 1 hr to give a yellow suspension. The result mixture was dropwise to a solution of 14-1 (70 mg, 158.14 μmol, 1 eq) and TEA (16.00 mg, 158.14 μmol, 22.01 uL, 1 eq) in DCM (5 mL) at 0° C. It was stirred at 0° C. for 2 hr to give a yellow solution. LCMS and TLC (PE:EtOAc=1:1, 3/1) showed the mixture was completed. The mixture was quenched with H₂O (8 mL), extracted with DCM (8 mL*3). The organic layers were washed with HCl (1N, 10 mL). The organic layer was separated. The organic layers were washed with Sat·NaHCO₃ (10 mL). The organic layer was separated and dried over sodium sulfate, filtered and concentrated under vacuum. The residue was purified by column chromatography (SiO₂, Petroleum ether/Ethyl acetate=100/0 to 80/20) to give 14-2. LC-MS (m/z): 567.2 [M+H]⁺

¹H NMR (400 MHz, CDCl₃) δ ppm 7.13-6.97 (m, 3H) 6.81-6.60 (m, 5H) 4.85-4.72 (m, 2H) 3.85-3.77 (m, 3H) 3.16-2.17 (m, 6H) 2.10 (br s, 3H) 1.88-1.80 (m, 6H) 1.73-1.62 (m, 8H) 0.31-0.21 (m, 9H)

To a solution of 14-2 (32 mg, 56.45 μmol, 1 eq) in THF (5 mL) were added TBAF (1 M, 62.10 uL, 1.1 eq) at −78° C. The mixture was stirred at −78° C. for 15 mins to give a yellow solution. TLC (PE:EtOAc=2:1) showed the mixture was completed. The mixture was added H₂O (5 mL), extracted with EtOAc (8 mL*3). The organic layers were dried over sodium sulfate, filtered and concentrated under vacuum. The residue was purified by column chromatography (SiO₂, Petroleum ether/Ethyl acetate=100/0 to 72/28) to give B-51. LC-MS (m/z): 495.3 [M+H]⁺. ¹H NMR (400 MHz, CDCl₃) δ ppm 7.18-6.95 (m, 3H) 6.84-6.58 (m, 5H) 4.73 (br s, 1H) 3.82 (br s, 3H) 3.31-3.17 (m, 1H) 3.13-2.83 (m, 1H) 2.75-2.47 (m, 1H) 2.40 (br s, 1H) 2.10 (br s, 3H) 1.97-1.80 (m, 8H) 1.68-1.53 (m, 8H) 1.32-1.03 (m, 1H) 0.90 (br s, 1H).

### Procedure 28: Compound A-41

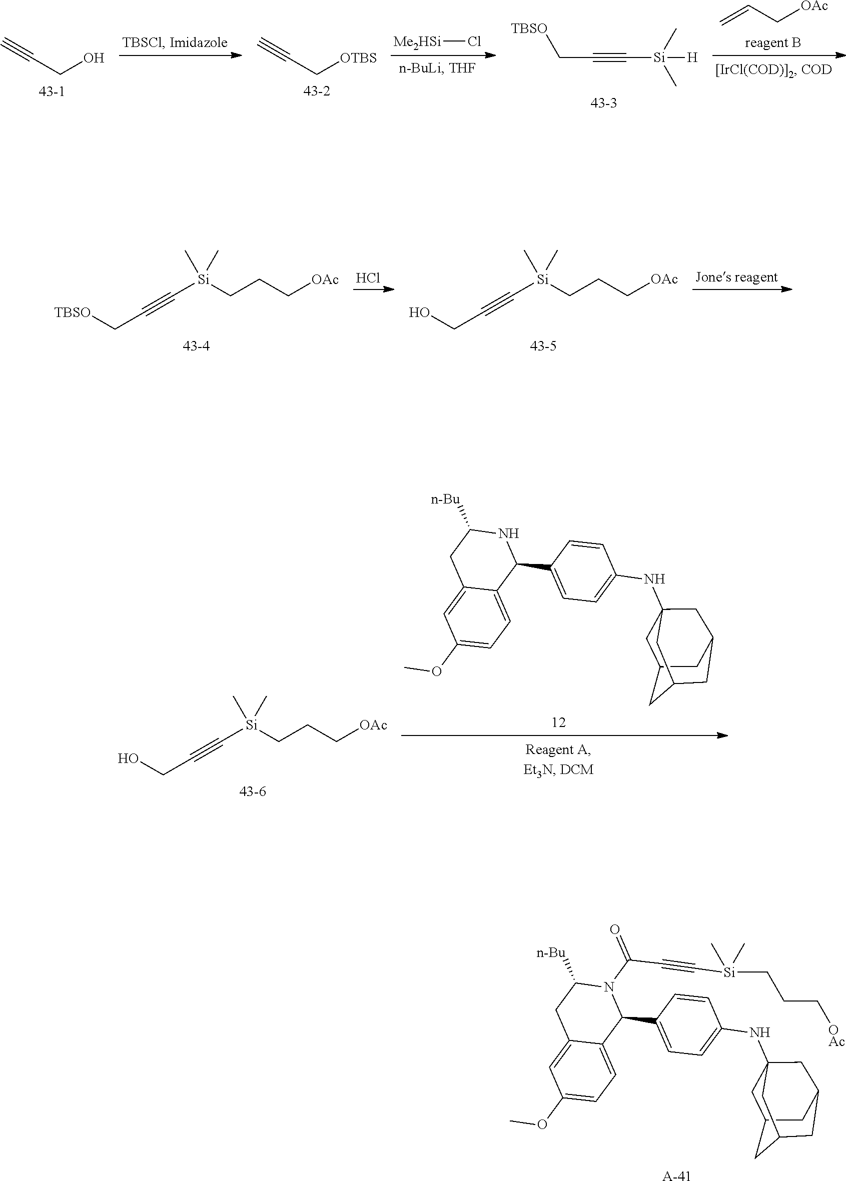

### General Procedure for the Preparation of Compound 43-2

To a solution of prop-2-yn-1-ol (15 g, 267.56 mmol, 15.81 mL, 1 eq) in DCM (150 mL) was added TBSCl (44.36 g, 294.31 mmol, 36.06 mL, 1.1 eq) and imidazole (27.32 g, 401.33 mmol, 1.5 eq) at 0° C. The mixture was stirred at 20° C. for 16 h. TLC (PE/EtOAc=10:1) showed the reaction was completed. The reaction mixture was washed with water, dried over Na₂SO₄, and was concentrated in vacuo. The crude product was distilled in vacuum (45° C., 0.2 atm/oil pump) to give compound 43-2 (35 g, 205.49 mmol, 76.80% yield).

### General Procedure for the Preparation of Compound 43-3

n-BuLi (2.5 M, 16.91 mL, 1.1 eq) was slowly added to compound 43-2 (6.55 g, 38.43 mmol, 7.79 mL, 1 eq) in THF (50 mL) under N₂. The mixture was stirred for 0.5 h at −78° C., then chloro(dimethyl)silane (4 g, 42.28 mmol, 1.1 eq) was added into the reaction. Then the reaction was stirred at 20° C. for 16 hours. TLC (PE/EtOAc=100:1) showed the reaction was completed. The resulting solution was poured into water and washed with EtOAc. The organic layer was dried over anhydrous sodium sulfate and concentrated under reduced pressure to afford the crude product. The crude product was purified by flash column on silica gel eluting with EtOAc/PE (0:1) to afford compound 43-3 (5.9 g, 25.82 mmol, 67.19% yield).

### General Procedure for the Preparation of Compound 43-4

To a dry 10 mL round bottomed flask, under N₂. chloroiridium (1Z,5Z)-cycloocta-1,5-diene (1.39 g, 2.07 mmol, 0.08 eq), (1Z,5Z)-cycloocta-1,5-diene (13.97 g, 129.12 mmol, 15.85 mL, 5 eq), compound 43-3 (5.9 g, 25.82 mmol, 1 eq) and allyl acetate (2.53 g, 25.31 mmol, 0.98 eq) were sequentially added. The reaction was stirred at 20° C. for 24 hours. TLC (PE/EtOAc=20:1) showed the reaction was completed. The reaction mixture were filtered and concentrated under reduced pressure to afford the crude product. The crude product was purified by silica column eluting with EtOAc/petroleum ether (0% to 2.5%) to afford compound 43-4 (3.5 g, 10.65 mmol, 41.25% yield).

### General Procedure for the Preparation of Compound 43-5

To a dry 10 mL round bottomed flask, under N₂. Compound 43-4 (3.13 g, 9.54 mmol, 1 eq) in THF (15 mL) was added HCl (939.80 mg, 9.54 mmol, 921.37 μL, 1 eq). The reaction was stirred at 0° C. for 16 hours. TLC (PE/EtOAc=3:1) showed the reaction was completed. The mixture was quenched by NaHCO₃ (2 M, aq., 10 mL), the organic layer was washed by NaCl (saturated, aq., 10 mL). The organic layer was dried over anhydrous sodium sulfate and concentrated under reduced pressure to afford the crude product. The crude product was purified by silica column eluting with EtOAc/petroleum ether (2% to 20%) to afford compound 43-5 (1.17 g, 5.46 mmol, 57.24% yield).

### General Procedure for the Preparation of Compound 43-6

Preparation of Jones reagent: To a solution of CrO₃ (169.35 mg, 1.69 mmol, 62.72 μL, 1.1 eq) in H₂O (610.22 mg, 33.87 mmol, 610.22 μL, 22 eq) was added H₂SO₄ (184.91 mg, 1.85 mmol, 100.49 μL, 1.2 eq) at 0° C. The mixture was stirred at 25° C. for 1 h. To a solution of compound 43-5 (330 mg, 1.54 mmol, 1 eq) in acetone (5 mL) was added Jones reagent at 0° C. The mixture was stirred at 25° C. for 3 h to give a black solution. TLC (PE/EtOAc=2:1) showed the reaction was completed. The mixture was diluted with IPA (3 mL) and then it was diluted with EA (15 ml*3) and H₂O (15 mL). The organic layers were dried over sodium sulfate, filtered and concentrated under vacuum. compound 43-6 was used next step without further purification.

### General Procedure for the Preparation of A-41

To a solution of 3-[3-acetoxypropyl(dimethyl)silyl]prop-2-ynoic acid (308.08 mg, 1.35 mmol, 1.2 eq) in DCM (3 mL) was added 2-chloro-1-methyl-pyridin-1-ium iodide (344.74 mg, 1.35 mmol, 1.2 eq), the mixture was stirred at 25° C. for 0.5 h. Then a solution of compound 12 (500 mg, 1.12 mmol, 1 eq) and TEA (136.54 mg, 1.35 mmol, 187.82 μL, 1.2 eq) in DCM (3 mL) was added dropwise at 0° C. The mixture was stirred at 0° C. for 1 h to give a yellow solution. LCMS and TLC (PE/EtOAc=1:1) showed the reaction was completed. The mixture was quenched by NH₄Cl (10 mL), the organic layer was washed by 1 M HCl (3 mL) and 1 M NHCO₃ (10 mL). The organic layer was dried over anhydrous sodium sulfate and concentrated under reduced pressure to afford the crude product. The crude product was purified by silica column eluting with EtOAc/petroleum ether (15% to 30% to 35%) to afford the crude product (0.601 g, 917.63 μmol, 81.60% yield). The purified crude product was purified again by pre-TLC (PE:EA=1:1) to afford 330 mg of product. The crude product was purified by reversed-phase HPLC (0.1% TFA condition) to give Compound A-41. LC-MS (m/z): 655.4 [M+H]⁺. ¹H NMR (400 MHz, CDCl₃) δ ppm 10.89 (br s, 1H), 7.40-7.52 (m, 2H), 7.28-7.36 (m, 1H), 7.20-7.26 (m, 2H), 6.77-6.84 (m, 1H), 6.68 (d, J=2.25 Hz, 1H), 6.20-6.35 (m, 1H), 4.57-4.70 (m, 1H), 3.97-4.12 (m, 2H), 3.81 (s, 3H), 3.00 (br dd, J=15.07, 4.31 Hz, 1H), 2.75 (br d, J=15.13 Hz, 1H), 2.06-2.09 (m, 3H), 1.79 (br s, 9H), 1.66 (br s, 6H), 1.38-1.45 (m, 5H), 1.22-1.30 (m, 4H), 0.85-0.90 (m, 3H), 0.71-0.78 (m, 2H), 0.24-0.35 (m, 5H).

### Procedure 29: Compound A-42

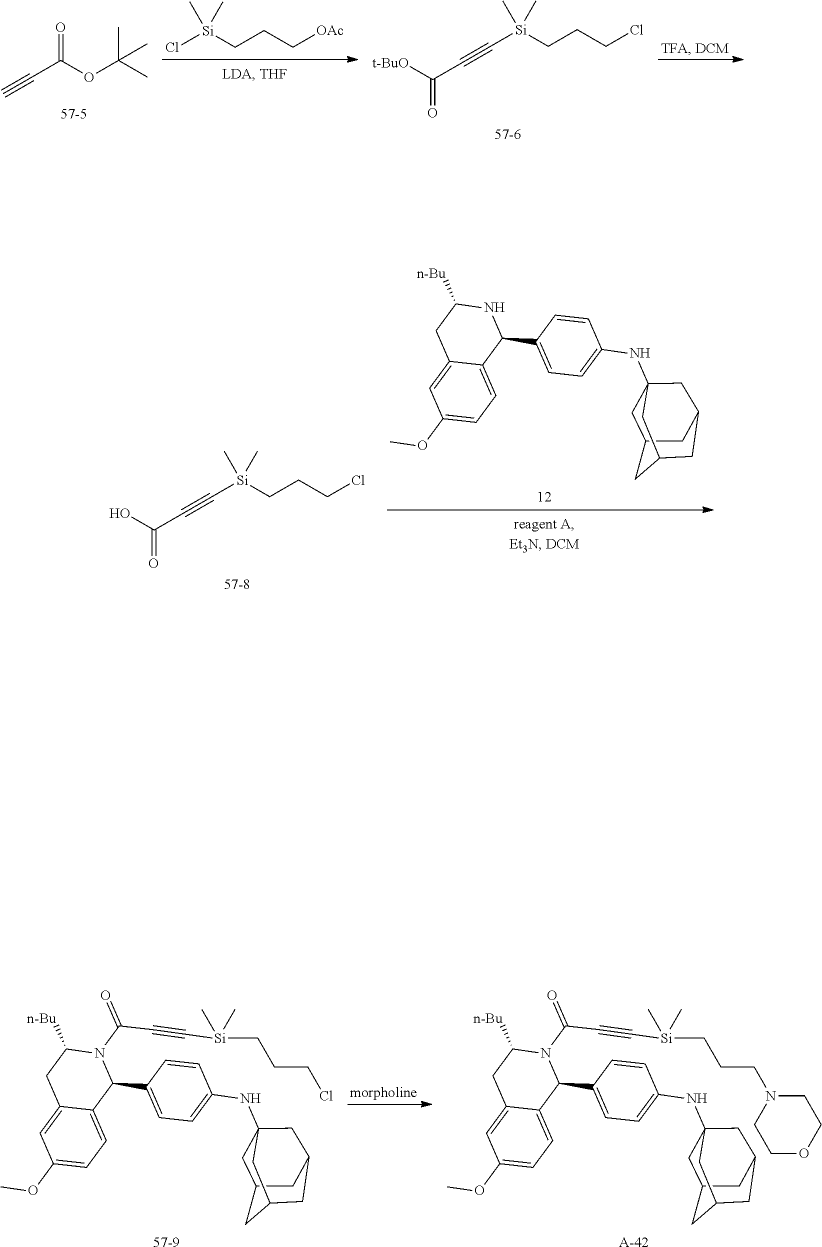

### General Procedure for the Preparation of Compound 57-6

Compound 57-5 (2.01 g, 15.94 mmol, 2.19 mL, 1 eq) was dissolved in anhydrous THF (20 mL) and the solution was cooled to −78° C. Under N2, with stirring. n-BuLi (2.5 M, 7.01 mL, 1.1 eq) was added to the above solution and the mixture was stirred for 30 min. chloro-(3-chloropropyl)-dimethyl-silane (3 g, 17.53 mmol, 1.1 eq) was then added and the resulting solution was stirred at −78° C. for 1 h and allowed to warm to 20° C. over a period of 2 h. TLC (PE/EtOAc=20:1) showed the reaction was completed. The reaction was cooled to 0° C., quenched with water (10 mL) was added to the crude reaction mixture. The reaction mixture was poured into a separatory funnel, the organic layer was separated and the aqueous layer was extracted with EA (4×20 mL). The combined organic layers were washed with brine (20 mL), dried over anhydrous Na₂SO₄, filtered, and concentrated in vacuo. The crude product was purified by silica column eluting with EtOAc/petroleum ether (1%) to afford 57-6 (2.8 g, 10.73 mmol, 67.36% yield).

### General Procedure for the Preparation of Compound 57-8

The mixture of 57-6 (0.4 g, 1.53 mmol, 1 eq) in TFA/DCM=95:5 (1.53 mmol, 3 mL, 1 eq) was stirred at 20° C. for 2 h. TLC (PE/EtOAc=1:1) showed the reaction was completed. The reaction mixture was concentrated under reduced pressure. The crude product was purified by silica column eluting with EtOAc/petroleum ether (15% to 40%) to afford 57-8 (0.202 g, 986.69 μmol, 64.34% yield).

### General Procedure for the Preparation of Compound 57-9

A solution of compound 12 (500 mg, 1.12 mmol, 1 eq) and 57-8 (253.23 mg, 1.24 mmol, 1.1 eq) in DCM (5 mL) was degassed with N2 for 15 min. And then EEDQ (333.69 mg, 1.35 mmol, 1.2 eq) was added. The reaction was stirred at 20° C. for 16 h. LCMS showed the reaction was completed. The reaction mixture was concentrated under reduced pressure. The crude product was purified by silica column eluting with EtOAc/petroleum ether (10% to 20%) to afford 57-9 (0.43 g, 681.07 μmol, 60.57% yield).

### General Procedure for the Preparation of Compound A-42

To a solution of 57-9 (780 mg, 1.24 mmol, 1 eq) in toluene (6 mL) was added morpholine (322.89 mg, 3.71 mmol, 326.15 μL, 3 eq) and NaI (555.55 mg, 3.71 mmol, 3 eq) under N2. The mixture was stirred at 110° C. for 72 hr to give a yellow solution. LCMS showed the reaction was completed. The reaction mixture was concentrated under reduced pressure. The residue was purified by prep-HPLC (HCl condition; column: Welch Xtimate C18 100*40 mm*3 μm; mobile phase: [water (10 mM HCl)-ACN]; B %: 40%-70%, 8.5 min) to give Compound A-42 (210 mg, 307.91 μmol, 24.92% yield). LC-MS (m/z): 682.4[M+H]⁺. ¹H NMR (CDCl₃, 400 MHz): δ=11.61 (br d, J=11.26 Hz, 1H), 11.15 (br d, J=10.13 Hz, 2H), 7.70 (br d, J=8.00 Hz, 2H), 7.41 (br d, J=8.25 Hz, 1H), 7.33 (br d, J=8.00 Hz, 2H), 6.85 (br d, J=8.25 Hz, 1H), 6.71 (d, J=1.50 Hz, 1H), 6.23 (s, 1H), 4.83-4.75 (m, 1H), 4.29-4.17 (m, 3H), 3.97 (br t, J=12.88 Hz, 2H), 3.81 (s, 3H), 3.68 (br d, J=12.01 Hz, 1H), 3.13-3.01 (m, 3H), 2.85-2.72 (m, 3H), 2.12 (br s, 3H), 2.07 (br s, 4H), 1.78 (s, 6H), 1.62 (br s, 6H), 1.51 (br s, 2H), 1.34 (br s, 1H), 1.25 (dt, J=13.66, 6.74 Hz, 3H), 1.04-0.93 (m, 1H), 0.84 (br t, J=6.57 Hz, 3H), 0.31 (br t, J=8.32 Hz, 3H), 0.15 ppm (d, J=18.64 Hz, 6H).

### Procedure 30: Compounds A-43 and B-71

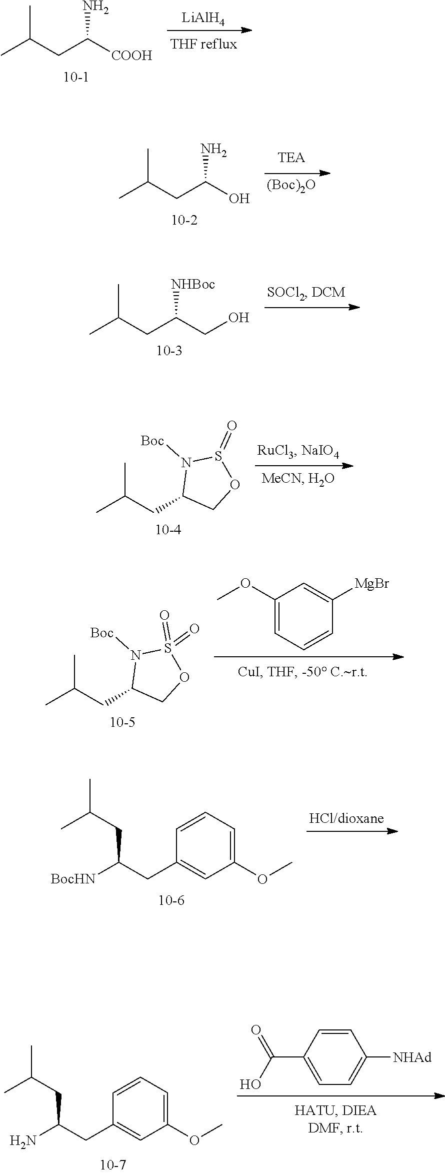

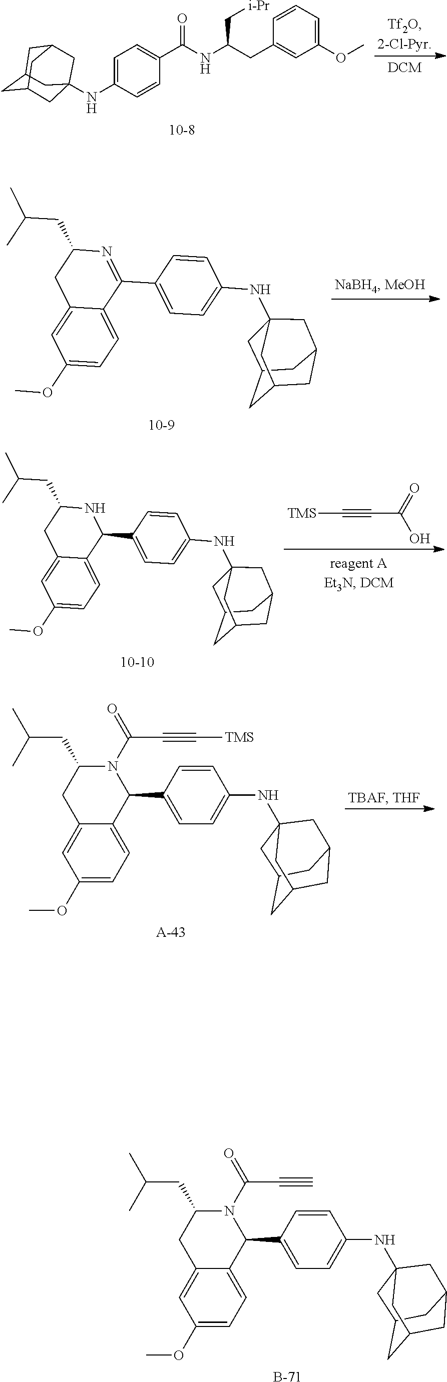

### General Procedure for the Preparation of 10-2

To a solution of LiAlH₄ (4.34 g, 114.35 mmol, 1.5 eq) in THF (400 mL) was added (2S)-2-amino-4-methyl-pentanoic acid (10 g, 76.24 mmol, 1 eq) at 0° C. The mixture was warmed to 25° C. and stirred for 1 h, then the mixture was warmed up to 70° C. and stirred for 12 h to give a white suspension. The reaction was monitored by NMR. Those four paralleled batches were quenched by the condition: H₂O (5 mL), 15% NaOH (5 mL) and H2O (15 mL), diluted with THF (100 mL) and dried Na₂SO₄, filtered and concentrated to give the crude product. The product was used for next step without further purification. To give 10-2 (8 g, crude). ¹H NMR (400 MHz, CDCl₃) δ=3.55 (dd, J=10.4 Hz 1H), 3.22 (dd, J=10.4 Hz 1H), 2.88 (m, 1H), 1.69 (dt, J=13.6 Hz 1H), 1.18 (m, 2H), 0.90 (dd, J=12.0 Hz 8H).

### General Procedure for the Preparation of 10-3

To a solution of 10-2 (8 g, 68.27 mmol, 1 eq) in DCM (150 mL) was added TEA (10.36 g, 102.40 mmol, 14.25 mL, 1.5 eq) and (Boc)₂O (13.41 g, 61.44 mmol, 14.11 mL, 0.9 eq) at 0° C. The mixture was warmed to 20° C. and stirred for 12 h to give a clear solution. TLC (PE/EtOAc=2:1) showed the reaction was completed. The reaction was quenched with H2O (50 mL). The separated aqueous layer was extracted with DCM (100 mL×2). The combined organic layers were washed with brine (100 mL) and dried over sodium sulfate, filtered and concentrated to give the crude product. The crude product was purified by flash column (SiO2, PE/EtOAc=0% to 20%) to give 10-3 (7.3 g, 33.59 mmol, 49.21% yield). ¹H NMR (400 MHz, CDCl₃) δ=4.61 (m, 1H), 3.58 (m, 3H), 2.69 (m, 1H), 1.65 (m, 1H), 1.43 (s, 9H), 1.29 (m, 3H), 0.91 (d, J=6.80 Hz 6H).

### General Procedure for the Preparation of 10-4

A solution of imidazole (13.40 g, 196.86 mmol, 5.86 eq) in DCM (150 mL) was cooled to 0° C. SOCl2 (6.95 g, 58.45 mmol, 4.24 mL, 1.74 eq) in DCM (36 mL) was added to the solution and the result suspension was allowed to warmed to 15° C. and stirred for 1 h at this temperature. Then the mixture was cooled to −78° C., a solution of 10-3 (7.3 g, 33.59 mmol, 1 eq) in DCM (150 mL) was added to the mixture drop wise. The result mixture was allowed to 20° C. and stirred for 12 h to give a yellow solution. TLC (PE:EtOAc=2:1) showed the reaction was completed. The reaction mixture was poured into water (200 mL) and extracted with DCM (150 mL×4). The combined organic layers were dried over Na₂SO₄, filtered and concentrated under reduced pressure. The product was used for next step without further purification to give 10-4 (9.7 g, crude). ¹H NMR (400 MHz, CDCl₃) δ=4.95 (m, 1H), 4.75 (m, 1H), 4.41 (d, J=8.80 Hz 1H), 4.05 (m, 2H), 2.03 (m, 1H), 1.53 (m, 10H), 1.43 (m, 2H), 0.95 (m, 6H).

### General Procedure for the Preparation of Compound 10-5

RuCl3.3H2O (48.15 mg, 184.16 μmol, 0.005 eq) was added to a stirred mixture of 10-4 (9.7 g, 36.83 mmol, 1 eq) in MeCN (100 mL) and H2O (66 mL) at 0° C., followed by portion wise addition of NaIO4 (8.67 g, 40.52 mmol, 2.25 mL, 1.1 eq). The mixture was allowed to warm to 20° C. and stirred for 12 h to give a black suspension. TLC (PE:EtOAc=4:1) showed the reaction was completed. The reaction was filtered and washed with EtOAc (100 mL). The filtrate was extracted with EtOAc (200 mL*2). The combined organic layers were washed with brine (100 mL) and dried over sodium sulfate, filtered and concentrated to give the crude product. The crude product was purified by flash column (eluting with: PE/EtOAc=0% to 6%) to give 10-5 (5.6 g, 20.05 mmol, 54.43% yield). ¹H NMR (400 MHz, CDCl₃) δ=4.63 (m, 1H), 4.31 (m, 2H), 1.75 (m, 2H), 1.60 (m, 2H), 1.55 (s, 9H), 0.95 (dd, J=12.4 Hz 6H).

### General Procedure for the Preparation of Compound 10-6

To a solution of CuI (37.63 mg, 197.60 μmol, 0.012 eq) was placed in a was added bromo-(3-methoxyphenyl)magnesium (1 M, 19.76 mL, 1.2 eq) at 0° C. under N2. The reaction was stirred 0° C. under N2 for 2 h. Then 10-5 (4.6 g, 16.47 mmol, 1 eq) in THF (50 mL) was added dropwise at −20° C. The reaction was allowed to stir at 20° C. for 12 h to give a yellow solution. TLC (eluting with: PE/EtOAc=6/1) showed the reaction was completed. The reaction was quenched with saturated citric acid (50 mL) and extracted with EtOAc (60 mL*3). The organic layers were dried over Na2SO4 and concentrated to give the crude product. The crude product was purified by flash column (eluting with: PE/EtOAc=0% to 4%) to give 10-6 (3.3 g, 8.37 mmol, 50.85% yield). ¹H NMR (400 MHz, CDCl₃) δ=7.19 (m, 1H), 6.75 (m, 3H), 4.25 (d, J=8.00 Hz 1H), 3.90 (m, 1H), 3.80 (s, 3H), 2.73 (d, J=5.60 Hz 2H), 1.68 (dt, J=12.8 Hz 1H), 1.41 (s, 9H), 1.23 (m, 2H), 0.88 (m, 6H).

### General Procedure for the Preparation of Compound 10-7

The 10-6 (3.3 g, 10.73 mmol, 1 eq) was dissolved in HCl/dioxane (4 M, 50 mL, 18.63 eq), the mixture was stirred at 25° C. for 12 h to give a yellow suspension. LCMS showed the desired MS. The mixture was concentrated under reduced pressure to afford the crude product. The product was used for next step without further purification to give 10-7 (2.8 g, crude, HCl). LC-MS (m/z): 207.9[M+H]⁺. ¹H NMR (400 MHz, MeOD) δ=7.28 (t, J=8.00 Hz 1H), 6.84 (m, 3H), 3.80 (s, 1H), 3.49 (t, J=6.80 Hz 1H), 2.89 (dd, J=6.80 Hz 2H), 1.74 (m, 1H), 1.47 (m, 2H), 0.95 (d, J=6.4 Hz 3H), 0.90 (d, J=6.8 Hz 3H).

### General Procedure for the Preparation of Compound 10-8

To a solution of 4-(1-adamantylamino)benzoic acid (1.22 g, 4.51 mmol, 1.1 eq) in DCM (20 mL) was added HATU (1.87 g, 4.92 mmol, 1.2 eq) and DIEA (1.59 g, 12.31 mmol, 2.14 mL, 3 eq). The mixture was stirred at 20° C. for 1 h. Then 10-7 (1 g, 4.10 mmol, 1 eq, HCl) was added. The reaction was stirred at 20° C. for 15 h to give a yellow solution. LCMS showed the desired MS. The mixture was concentrated under reduced pressure to afford the crude product. The crude product was purified by flash column (eluting with: PE/EtOAc=0% to 28%) to give 10-8 (1.28 g, 2.78 mmol, 67.74% yield). LC-MS (m/z): 461.3[M+H]⁺. ¹H NMR (400 MHz, CDCl₃) 7.51 (d, J=8.80 Hz 2H), 7.2 (m, 1H), 6.76 (m, 5H), 5.66 (d, J=6.00 Hz 1H), 4.46 (d, J=6.40 Hz 1H), 3.75 (s, 3H), 2.86 (d, J=5.60 Hz 2H), 2.13 (m, 3H), 1.95 (d, J=2.40 Hz 6H), 1.69 (s, 8H), 1.35 (t, J=7.20 Hz 2H), 0.90 (d, J=6.4 Hz 6H).

### General Procedure for the Preparation of Compound 10-9

To a solution of 10-8 (1.28 g, 2.78 mmol, 1 eq) in DCM (20 mL) was added Tf₂O (2.35 g, 8.34 mmol, 1.38 mL, 3 eq) and 2-chloropyridine (946.56 mg, 8.34 mmol, 788.80 μL, 3 eq) at −78° C. under nitrogen atmosphere. The mixture was stirred at −78° C. for 15 min. Then the mixture was stirred at −0° C. for 15 min. The mixture was stirred at 20° C. for 1 h to give a yellow solution. LCMS showed the desired MS. The reaction mixture was poured into water (20 mL) and extracted with DCM (30 mL×2). The combined organic layers were washed with saturated Na₂CO₃ aq. (30 mL×2), dried over Na₂SO₄, filtered and concentrated under reduced pressure. The crude product was purified by flash column (eluting with: PE/EtOAc=0% to 80%) to give 10-9 (1.16 g, 2.62 mmol, 94.31% yield). ¹H NMR (400 MHz, CDCl₃) 7.55 (m, 3H), 6.87 (m, 4H), 4.25 (s, 1H), 3.95 (s, 3H), 3.30 (dd, J=16.0 Hz 1H), 2.87 (dd, J=16.0 Hz 1H), 2.16 (m, 3H), 2.01 (d, J=2.00 Hz 6H), 1.71 (s, 9H), 1.31 (m, 2H), 0.96 (dd, J=14.8 Hz 6H).

### General Procedure for the Preparation of Compound 10-10

To a solution of 10-9 (660 mg, 1.49 mmol, 1 eq) in MeOH (7 mL) was added NaBH₄ (141.02 mg, 3.73 mmol, 2.5 eq). The reaction was stirred at 50° C. for 10 min to give a yellow solution. The reaction was added NaBH₄ (141.03 mg, 3.73 mmol, 2.5 eq). The reaction was stirred at 50° C. for 20 min to give a yellow solution. LCMS showed the reaction was completed. The crude products were combined for work up. The reaction mixture was poured into saturated NaHCO₃ aq. (15 mL) and extracted with EtOAc (15 mL×3). The combined organic layers were dried over Na₂SO₄, filtered and concentrated under reduced pressure. The crude products on were combined for further purification. The crude product was purified by flash column (SiO₂, eluting with: PE/EtOAc=0% to 60%) to give 10-10 (100 mg).

### General Procedure for the Preparation of Compound A-43

To a solution of 3-trimethylsilylprop-2-ynoic acid (12.79 mg, 89.96 μmol, 1 eq) in DCM (2 mL) was added 2-chloro-1,6-dimethylpyridin-1-ium (12.83 mg, 89.96 μmol, 1 eq). The reaction was stirred at 10° C. for 0.5 hr. Then the mixture was added dropwise the solution of 10-10 (40 mg, 89.96 μmol, 1 eq) and TEA (9.10 mg, 89.96 μmol, 12.52 μL, 1 eq) in DCM (2 mL) at 0° C. The reaction stirred at 0° C. for 0.5 hr to give a yellow solution. LCMS showed the reaction was not completed. The reaction was added TEA (6 μL). The reaction stirred at 10° C. for 3 hr to give a yellow solution. TLC (PE:EtOAc=2:1) showed the reaction was not completed, a little of R1 remained. The reaction was basified with saturated NH₄Cl aq. (5 mL), extracted with DCM (5 mL×3). The combined organic layers were dried over Na₂SO₄, filtered and concentrated under reduced pressure. The crude product was purified by flash column (SiO₂, eluting with: PE/EtOAc=0% to 25%) to give Compound A-43 (12.78 mg, 22.47 μmol, 24.97% yield). LC-MS (m/z): 569.1 [M+H]⁺. ¹H NMR (400 MHz, CHLOROFORM-d) δ ppm 7.53-7.28 (m, 3H) 7.26-6.95 (m, 2H) 6.87-6.62 (m, 2H) 6.46-6.02 (m, 1H) 4.80 (br s, 1H) 3.87-3.74 (m, 3H) 3.11-2.67 (m, 2H) 2.10-1.96 (m, 2H) 1.87 (br s, 2H) 1.79 (br s, 4H) 1.61 (br s, 5H) 1.46-1.11 (m, 6H) 1.03-0.94 (m, 3H) 0.84-0.74 (m, 3H) 0.31-0.06 (m, 9H).

### General Procedure for the Preparation of Compound B-71

To a solution of A-43 (70 mg, 123.05 μmol, 1 eq) in THF (2 mL) was added TBAF (1 M, 123.05 μL, 1 eq) −78° C. The reaction was stirred at −78° C. for 0.5 hr to give a colorless solution. LCMS showed the reaction was completed. The reaction mixture was poured into H2O (5 mL) and extracted with EtOAc (5 mL×3). The combined organic layers were dried over Na₂SO₄, filtered and concentrated under reduced pressure. The crude product was purified by flash column (SiO₂, eluting with: PE/EtOAc=0% to 25%) to give Compound B-71 (36.16 mg, 71.57 μmol, 58.16% yield). LC-MS (m/z): 497.1 [M+H]⁺. ¹H NMR (400 MHz, CHLOROFORM-d) δ ppm 7.29 (s, 1H) 7.23 (d, J=8.38 Hz, 1H) 7.01-6.89 (m, 2H) 6.81 (ddd, J=13.13, 8.38, 2.50 Hz, 1H) 6.69 (br dd, J=5.63, 2.38 Hz, 3H) 6.32-6.21 (m, 1H) 4.81-4.48 (m, 1H) 3.81 (d, J=5.88 Hz, 3H) 3.14-2.67 (m, 3H) 2.10-2.02 (m, 3H) 1.99 (br d, J=4.00 Hz, 1H) 1.82 (dd, J=9.76, 2.25 Hz, 6H) 1.70-1.61 (m, 6H) 1.50-1.39 (m, 1H) 1.29-1.11 (m, 1H) 1.00-0.94 (m, 3H) 0.83-0.74 (m, 3H).

### Procedure 31: Compounds A-44 and B-60

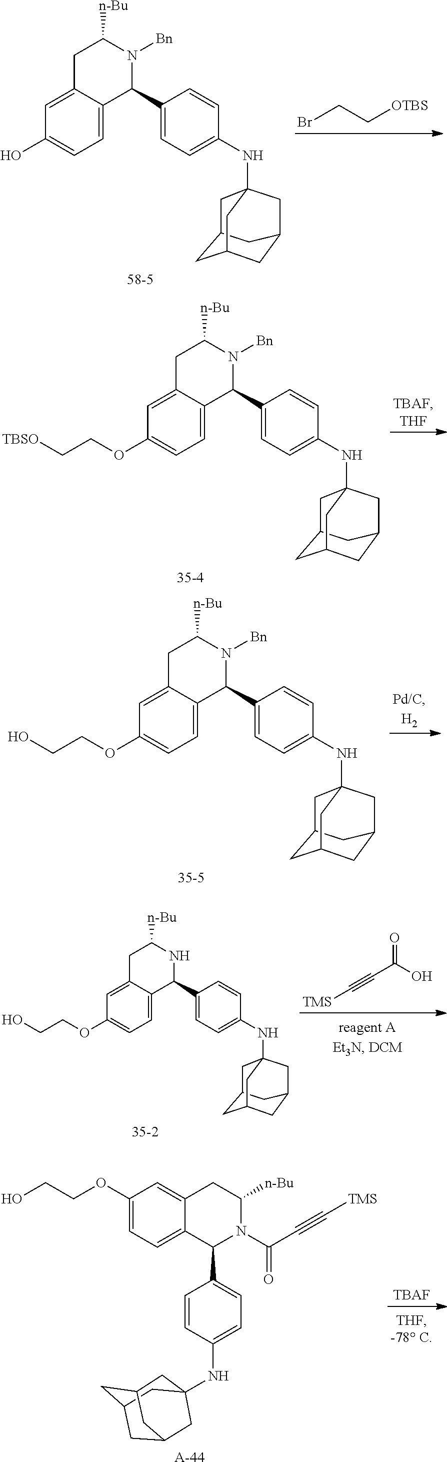

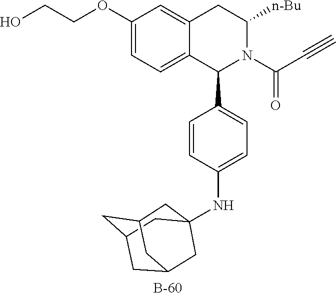

### General Procedure for the Preparation of Compound 35-4

To a solution of 58-5 (0.45 g, 864.15 μmol, 1 eq) and 2-bromoethoxy-tert-butyl-dimethyl-silane (310.09 mg, 1.30 mmol, 1.5 eq) in DMF (10 mL) was added K₂CO₃ (597.15 mg, 4.32 mmol, 5 eq) and KI (14.34 mg, 86.41 μmol, 0.1 eq), then the mixture was stirred at 80° C. for 16 h. LCMS and TLC (PE:EtOAc=3:1) showed the mixture was completed. The reaction mixture were filtered and concentrated under reduced pressure to afford the crude product. The crude product was purified by silica column eluting with EtOAc/petroleum ether (1% to 6%) to afford 35-4 (0.41 g, 603.78 μmol, 69.87% yield).

### General Procedure for the Preparation of Compound 35-5

35-4 (0.41 g, 603.78 μmol, 1 eq) was dissolved in a mixture of THF (10 mL) under N₂. To the mixture was added TBAF (1 M, 784.91 μL, 1.3 eq), the mixture was stirred at 20° C. for 1 h. TLC (PE:EtOAc=3:1) showed the mixture was completed. The mixture was quenched by NH₄Cl (10 mL), the organic layer was washed by NaCl (saturated, aq., 10 mL). The organic layer was dried over anhydrous sodium sulfate and concentrated under reduced pressure to afford the crude product. The crude product was purified by silica column eluting with EtOAc/petroleum ether (2% to 20%) to afford 35-5 (0.26 g, 460.34 μmol, 76.24% yield).

### General Procedure for the Preparation of 35-2

35-5 (170 mg, 300.99 μmol, 1 eq) was dissolved in MeOH (5 mL), Pd/C (17 mg, 30.10 μmol, 0.1 eq) was added into the reaction mixture. The system was flushed with H₂Pd/C (17 mg, 30.10 μmol, 0.1 eq) and then evacuated and backfilled with H₂ (17 mg, 30.10 μmol, 0.1 eq) (3 times). The mixture was stirred at 20° C. for 16 h. LCMS and TLC (PE:EtOAc=3:1) showed the mixture was completed. The mixture was filtered and concentrated under reduced pressure to afford the crude product. The crude product was used to the next step without purification.

### General Procedure for the Preparation of A-44

To a solution of 3-trimethylsilylprop-2-ynoic acid (37.00 mg, 260.18 μmol, 0.95 eq) in DCM (3 mL) was added 2-chloro-1-methyl-pyridin-1-ium; iodide (76.97 mg, 301.26 μmol, 1.1 eq), the mixture was stirred at 25° C. for 0.5 h. Then a solution of 35-2 (130 mg, 273.87 μmol, 1 eq) and TEA (33.26 mg, 328.65 μmol, 45.74 μL, 1.2 eq) in DCM (4 mL) was added dropwise at 0° C. The mixture was stirred at 0° C. for 1 h to give a yellow solution. LCMS and TLC (PE:EtOAc=1:1) showed the mixture was completed. The crude product was purified by flash column on silica gel eluting with EtOAc/PE (1:10 to 1:3) and dried by lyophilized to afford A-44 (110 mg, 183.67 μmol, 67.07% yield). LC-MS (m/z): 599.3 [M+H]⁺. ¹H NMR (400 MHz, CDCl₃) δ ppm 7.28-7.45 (m, 1H), 6.74-6.92 (m, 5H), 6.56-6.69 (m, 2H), 5.88-6.12 (m, 1H), 4.68-4.90 (m, 2H), 4.44-4.62 (m, 1H), 3.87-3.98 (m, 2H), 3.68 (q, J=5.13 Hz, 2H), 2.73-3.06 (m, 3H), 2.01 (br s, 4H), 1.78 (br s, 7H), 1.60 (br s, 8H), 1.01-1.51 (m, 9H), 0.74-0.88 (m, 4H), 0.25 (s, 6H), 0.06 (s, 3H).

### General Procedure for the Preparation of B-60

To a solution of A-44 (80 mg, 133.58 μmol, 1 eq) in THF (5 mL) was added TBAF (1 M, 160.30 μL, 1.2 eq), and then the mixture was stirred at −78° C. for 2 h. LCMS and TLC (PE:EtOAc=1:1) showed the mixture was completed. The mixture was quenched by NH₄Cl (10 mL), the organic layer was washed by NaCl (saturated, aq., 10 mL). The organic layer was dried over anhydrous sodium sulfate and concentrated under reduced pressure to afford the crude product. The crude product was purified by flash column on silica gel eluting with EtOAc/PE (1:10 to 1:3), then prep-HPLC (basic condition) and lyophilized to afford B-60 (18 mg, 34.17 μmol, 25.58% yield). LC-MS (m/z): 527.3 [M+H]⁺. ¹H NMR (400 MHz, CDCl₃) δ ppm 7.29-7.48 (m, 1H), 6.49-6.92 (m, 6H), 5.89-6.18 (m, 1H), 4.69-4.99 (m, 2H), 4.27-4.66 (m, 1H), 3.87-4.01 (m, 2H), 3.61-3.74 (m, 2H), 3.42-3.50 (m, 2H), 2.78-3.07 (m, 1H), 2.62-2.75 (m, 1H), 2.01 (br s, 3H), 1.79 (br d, J=4.38 Hz, 6H), 1.42-1.66 (m, 6H), 1.02-1.30 (m, 5H), 0.72-0.89 (m, 3H).

### Procedure 32: Compound A-45

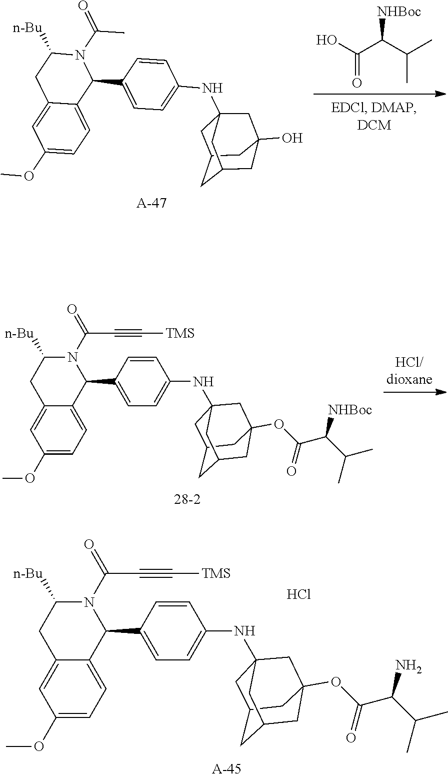

### General Procedure for the Preparation of Compound 28-2

To a solution of A-47 (100 mg, 170.98 μmol, 1 eq) in DCM (4 mL) were added (2S)-2-(tert-butoxycarbonylamino)-3-methyl-butanoic acid (55.72 mg, 256.47 μmol, 1.5 eq), EDCI (196.66 mg, 1.03 mmol, 6 eq) and DMAP (62.67 mg, 512.94 μmol, 3 eq). The reaction was stirred at 40° C. for 14 hr to give a yellow solution. LCMS showed the reaction was not completed. The reaction was stirred at 40° C. for 14 hr to give a yellow solution. LCMS showed the reaction was not completed, most of R1 remained. The reaction was basified with H₂O (5 mL), extracted with DCM (5 mL×3). The combined organic layers were dried over Na₂SO₄, filtered and concentrated under reduced pressure. The residue was purified by prep-TLC (PE/EtOAc=2:1) to give 28-2 (10 mg, crude).

### General Procedure for the Preparation of Compound A-45

To a solution of 28-2 (10 mg, 12.75 μmol, 1 eq) in HCl/dioxane (0.5 mL). The reaction was stirred at 10° C. for 1 hr to give light yellow solution. LCMS showed the reaction was completed. The reaction mixture was concentrated directly. The residue was purified by prep-HPLC [water (0.05% HCl)-ACN]; B %: 25%-55%, 8.5 min. The afforded flows were combined, concentrated to remove most of CH₃CN and lyophilized to give A-45 (0.82 mg, 1.13 μmol, 8.86% yield, HCl). LC-MS (m/z): 684.4 [M+H]⁺. ¹H NMR (400 MHz, METHANOL-d₄) δ ppm 7.59-7.20 (m, 5H) 6.96-6.74 (m, 2H) 6.50-6.03 (m, 1H) 4.16-3.81 (m, 1H) 3.80-3.77 (m, 3H) 3.27-3.11 (m, 1H) 3.01-2.85 (m, 1H) 2.45 (br s, 2H) 2.36-2.05 (m, 8H) 1.88 (br s, 4H) 1.69-1.54 (m, 4H) 1.32 (br d, J=6.53 Hz, 6H) 1.06 (dd, J=6.78, 1.51 Hz, 6H) 0.90 (br t, J=6.02 Hz, 3H) 0.33-0.02 (m, 9H).

### Procedure 33: Compounds A-46 and B-66

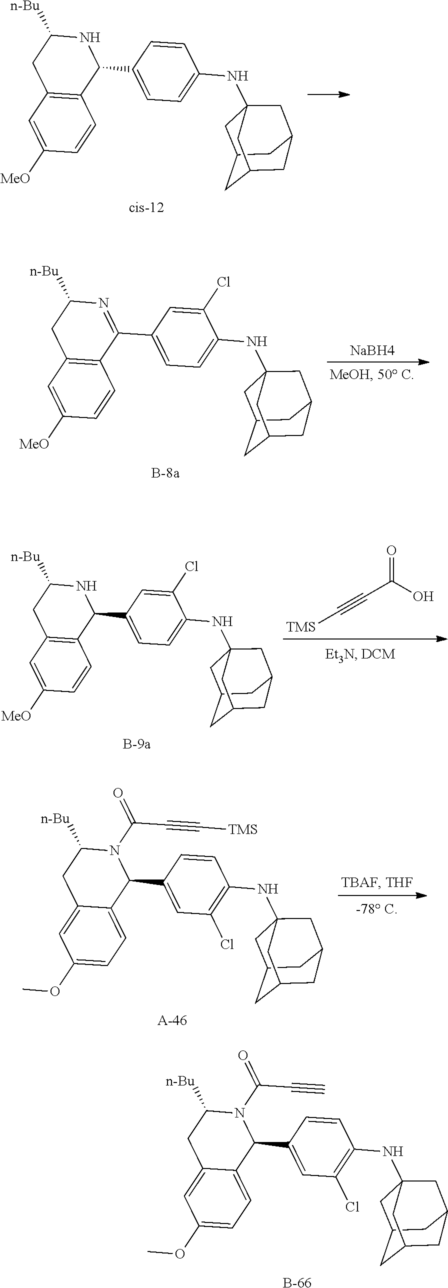

### General Procedure for the Preparation of B-8a

TCCA (36.59 mg, 157.43 μmol, 0.35 eq) was added into a solution of cis-12 (200.00 mg, 449.79 μmol, 1 eq) in DCM (5 mL) at 20° C. for 12 hours. LCMS and TLC (PE:EtOAc=3:2) showed the mixture was completed. The reaction mixture was quenched with H₂O (30 mL) and extracted with EtOAc (20 mL*3). The organic layers were dried over Na₂SO₄, filtered and concentrated to give the crude product. The residue was purified by column chromatography (SiO₂, Petroleum ether/Ethyl acetate=20/1 to 4/1) to give B-8a (65 mg, 113.36 μmol, 20.65% yield).

### General Procedure for the Preparation of B-9a

To a solution of B-8a (700 mg, 1.58 mmol, 1 eq) in MeOH (7 mL) was added NaBH₄ (179.49 mg, 4.74 mmol, 3 eq) at 50° C. The mixture was stirred at 50° C. for 1 h to give a yellow solution. LCMS and TLC (PE:EtOAc=2:1) showed the mixture was completed. The mixture was pour into H₂O (5 mL) and extracted with DCM (5 mL*4). The residue was purified by column chromatography (SiO₂, Petroleum ether/Ethyl acetate=20/1 to 4/1) to give B-9a (493 mg, 1.03 mmol, 65.07% yield).

### General Procedure for the Preparation of A-46

To a solution of 3-trimethylsilylprop-2-ynoic acid (33.84 mg, 237.95 μmol, 1.2 eq) in DCM (3 mL) was added 2-chloro-1-methyl-pyridin-1-ium; iodide (60.79 mg, 237.95 μmol, 1.2 eq), the mixture was stirred at 25° C. for 0.5 h. Then a solution of B-9a (95 mg, 198.29 μmol, 1 eq) and TEA (24.08 mg, 237.95 μmol, 33.12 μL, 1.2 eq) in DCM (5 mL) was added dropwise at 0° C. The mixture was stirred at 0° C. for 1 h to give a yellow solution. LCMS and TLC (PE:EtOAc=7:1) showed the mixture was completed. The mixture was quenched by NH₄Cl (saturated, aq., 10 mL), the organic layer was washed by HCl (1 M, 1 mL), and NaHCO₃ (saturated, aq., 5 mL). The organic layer was dried over anhydrous sodium sulfate and concentrated under reduced pressure to afford the crude product. The crude product was purified by flash column on silica gel eluting with 15% EtOAc in petroleum ether to afford product which was dried by lyophilized to afford A-46 (80 mg, 132.60 μmol, 66.87% yield). LC-MS (m/z): 603.3 [M+H]⁺. ¹H NMR (400 MHz, CDCl₃) δ ppm 7.28 (br s, 1H), 7.21 (d, J=8.28 Hz, 1H), 7.00-7.08 (m, 1H), 6.84-6.94 (m, 2H), 6.81 (td, J=7.84, 2.38 Hz, 1H), 6.68-6.72 (m, 1H), 6.14-6.21 (m, 1H), 4.52-4.63 (m, 1H), 4.05 (br s, 1H), 3.82 (s, 3H), 3.09 (br dd, J=15.18, 3.89 Hz, 1H), 2.91 (br dd, J=14.81, 5.02 Hz, 1H), 2.73 (br d, J=15.56 Hz, 1H), 2.10 (br s, 3H), 1.90 (br s, 6H), 1.64-1.77 (m, 7H), 1.50-1.59 (m, 9H), 1.11-1.37 (m, 5H), 0.80-0.91 (m, 3H), 0.24-0.31 (m, 6H).

### General Procedure for the Preparation of B-66

To a solution of A-46 (60 mg, 99.45 μmol, 1 eq) in THF (5 mL) was added TBAF (1 M, 119.34 μL, 1.2 eq) and then the mixture was stirred at −78° C. for 1 h. LCMS and TLC (PE:EtOAc=6:1) showed the mixture was completed. The mixture was quenched by NH₄Cl (10 mL), the organic layer was washed by NaCl (saturated, aq., 10 mL). The organic layer was dried over anhydrous sodium sulfate and concentrated under reduced pressure to afford the crude product. The crude product was purified by flash column on silica gel eluting with 15% EtOAc in petroleum ether to afford product which was dried by lyophilized to afford B-66 (30 mg, 56.48 μmol, 56.80% yield). LC-MS (m/z): 531.1 [M+H]⁺. ¹H NMR (400 MHz, CDCl₃) δ ppm 7.30 (s, 1H), 7.20 (d, J=8.28 Hz, 1H), 6.99-7.03 (m, 1H), 6.78-6.94 (m, 3H), 6.71 (s, 1H), 6.13-6.24 (m, 1H), 4.45-4.65 (m, 1H), 4.02-4.15 (m, 1H), 3.82 (d, J=5.27 Hz, 3H), 2.93-3.18 (m, 1H), 2.66-2.86 (m, 2H), 2.05-2.15 (m, 3H), 1.91 (dd, J=10.16, 2.38 Hz, 6H), 1.62-1.75 (m, 7H), 1.47-1.61 (m, 10H), 1.13-1.34 (m, 6H), 0.78-0.94 (m, 4H)

### Procedure 34: Compounds A-47 and B-13

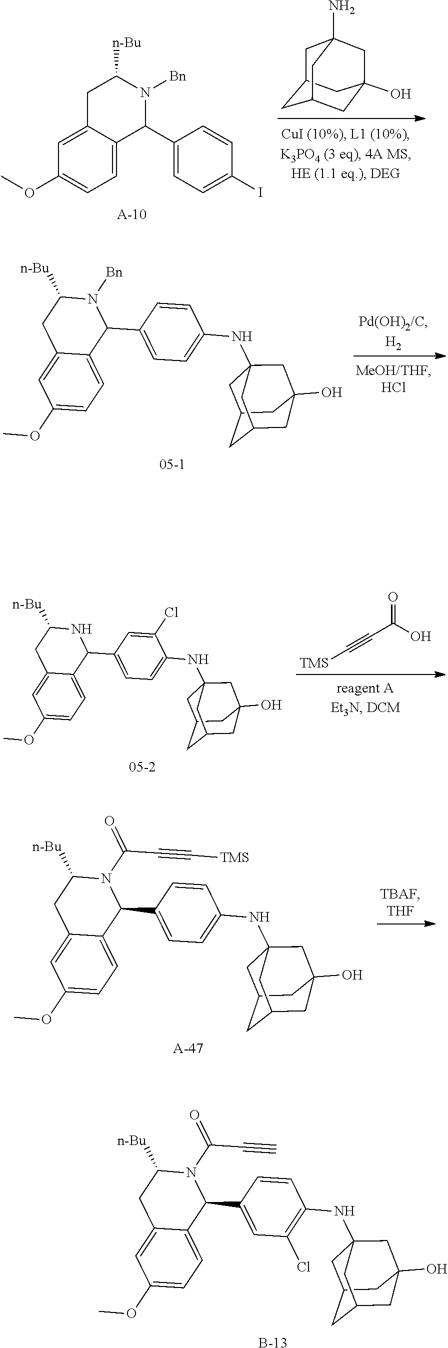

### General Procedure for the Preparation of Compound 05-1

A solution of CuI (37.24 mg, 195.53 μmol, 0.1 eq), N′,N′-diphenyl-1H-pyrrole-2-carbohydrazide (L1, 54.22 mg, 195.53 μmol, 0.1 eq), K₃PO₄ (1.25 g, 5.87 mmol, 3 eq), 4A MS (1 g, 1.96 mmol, 1 eq), diethyl 2,6-dimethyl-1,4-dihydropyridine-3,5-dicarboxylate (49.53 mg, 195.53 μmol, 0.1 eq), A-10 (1 g, 1.96 mmol, 1 eq) and (5R,7S)-3-aminoadamantan-1-ol (1.64 g, 9.78 mmol, 5 eq) and DEG (10 mL) and a magnetic stir bar. The vessel was sealed with a septum and bubbled with N₂ for 1 hours. The reaction mixture was stirred at 80° C. for 12 h to give brown suspension. TLC (PE/EtOAc=0:1) showed the reaction were completed. The reaction was quenched with NH₄OH (15 mL) and stirred at 20° C. for 0.5 hours. Then the reaction mixture was filtered and extracted by EtOAc (15 mL×3). The combined organic layers were dried over anhydrous sodium sulfate, filtered and concentrated under reduced pressure to afford the crude product. The crude product was purified by flash column (SiO₂, Petroleum ether/Ethyl acetate=100/0 to 75/25) to give 05-1 (1 g, 1.82 mmol, 92.86% yield).

### General Procedure for the Preparation of Compound 05-2

To a solution of 05-1 (500 mg, 907.82 μmol, 1 eq) in THF (2 mL)/MeOH (10 mL) was added Pd(OH)₂ (79.17 mg, 56.38 μmol, 0.0621 eq) and HCl (12 M, 276.88 μL, 3.66 eq) under N₂. The suspension was degassed under vacuum and purged with H₂ several times. The mixture was stirred under H₂ (1.83 mg, 907.82 μmol, 1 eq) at 20° C. for 16 hours to give a black suspension. LCMS showed the reaction was completed. The reaction mixture was filtered on Celite and washed with MeOH (10 mL). The filtrate was concentrated to give the crude product. The residue was diluted with DCM (10 mL) and H₂O (10 mL), extracted with DCM (15 mL×2). The organic layers were dried over sodium sulfate, filtered and concentrated under vacuum. The crude product was purified by flash column (SiO₂, petroleum ether/ethyl acetate=100/0 to 0/100) to give 05-2 (120 mg, 260.50 μmol, 28.70% yield).

### General Procedure for the Preparation of Compound A-47

To a solution of 3-trimethylsilylprop-2-ynoic acid (21.61 mg, 151.96 μmol, 1 eq) in DCM (0.5 mL) were added 2-chloro-1-methyl-pyridin-1-ium; iodide (38.82 mg, 151.96 μmol, 1 eq) The mixture was stirred at 20° C. for 0.5 hr to give a yellow suspension. Then a solution of 05-2 (70 mg, 151.96 μmol, 1 eq) and Et₃N (15.38 mg, 151.96 μmol, 21.15 μL, 1 eq) in DCM (0.5 mL) was dropwise. It was stirred at 0° C. for 1 hr to give a yellow solution. LCMS showed the reaction was not completed. It was stirred at 20° C. for 12 hr to give a yellow solution. TLC (PE/EtOAc=0:1) showed the reaction was not completed, a little of R1 remained. The reaction was basified with saturated NH₄Cl aq. (5 mL), extracted with DCM (5 mL×3). The combined organic layers were dried over Na₂SO₄, filtered and concentrated under reduced pressure. The crude product was purified by flash column (SiO₂, Petroleum ether/Ethyl acetate=100/0 to 20/80) to give A-47 (23.4 mg, 39.41 μmol, 25.93% yield). LC-MS (m/z): 585.7 [M+H]⁺. ¹H NMR (400 MHz, CHLOROFORM-d) δ ppm 7.33-7.28 (m, 1H) 7.24 (br d, J=8.63 Hz, 1H) 6.99 (br s, 1H) 6.83-6.68 (m, 3H) 6.27-6.21 (m, 1H) 6.27-6.21 (m, 1H) 4.59 (br s, 1H) 3.84-3.79 (m, 3H) 3.10-2.85 (m, 1H) 2.73 (br d, J=13.76 Hz, 1H) 2.23 (br d, J=11.38 Hz, 2H) 1.77 (br s, 2H) 1.72 (br s, 5H) 1.66-1.59 (m, 6H) 1.49 (br d, J=13.01 Hz, 3H) 1.26 (br s, 3H) 0.88-0.83 (m, 2H) 0.30-0.07 (m, 9H).

### General Procedure for the Preparation of Compound B-13

To a solution of A-47 (40 mg, 68.39 μmol, 1 eq) in THF (1 mL) was added TBAF (1.0 M, 75.23 μL, 1.1 eq) at −78° C. The reaction was allowed to stir at −78° C. for 0.5 hr to give a yellow solution. LCMS and TLC (PE:EtOAc=0:1) showed the reaction were completed. The reaction mixture was poured into H₂O (5 mL) and extracted with EtOAc (5 mL×3). The combined organic layers were dried over Na₂SO₄, filtered and concentrated under reduced pressure. The crude product was purified by flash column (SiO2, PE to 80% EtOAc in PE) to give B-13 (30.35 mg, 58.31 μmol, 85.26% yield). LC-MS (m/z): 513.5 [M+H]⁺. ¹H NMR (400 MHz, chloroform-d) δ ppm 7.30 (d, J=8.28 Hz, 1H) 7.23 (d, J=8.28 Hz, 1H) 6.93 (dd, J=12.80, 8.53 Hz, 2H) 6.81 (ddd, J=13.93, 8.28, 2.64 Hz, 1H) 6.72-6.62 (m, 3H) 6.24 (d, J=19.32 Hz, 1H) 4.66-4.44 (m, 1H) 3.82 (d, J=5.02 Hz, 3H) 3.21-2.60 (m, 3H) 2.28 (br s, 2H) 1.80-1.71 (m, 7H) 1.66 (br d, J=5.77 Hz, 4H) 1.55-1.50 (m, 2H) 1.33-1.14 (m, 6H) 0.89-0.81 (m, 3H).

### Procedure 35: Compounds A-48 and B-84

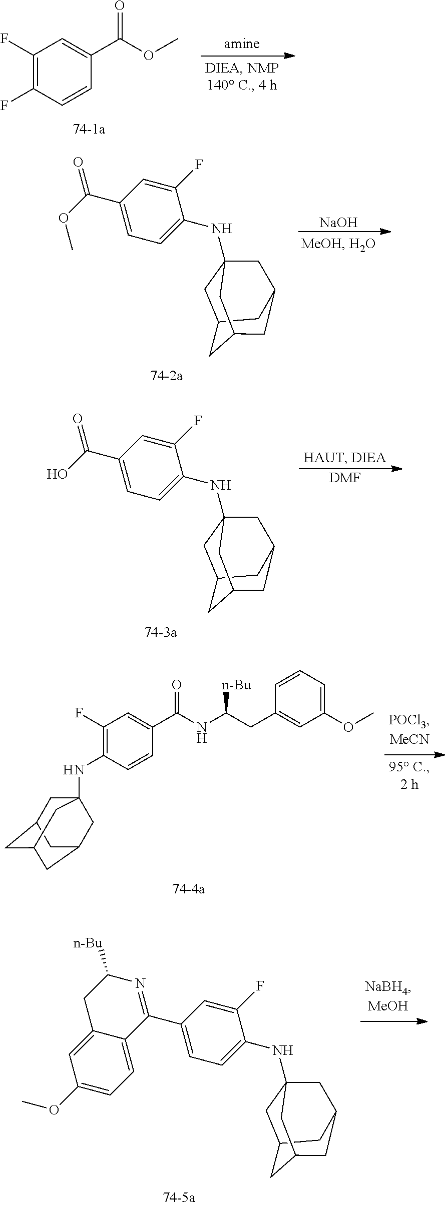

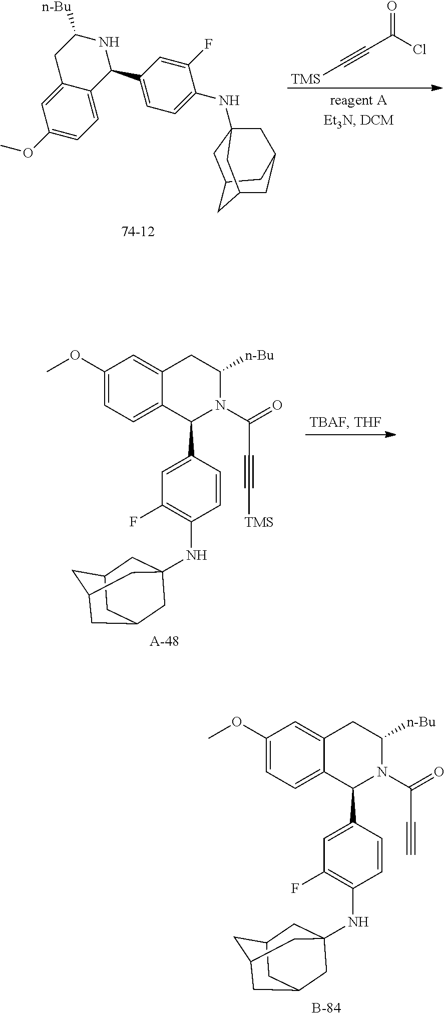

### General Procedure for the Preparation of Compound 74-2a

To a solution of 74-1a (5 g, 29.05 mmol, 1 eq) in NMP (40 mL) were added adamantan-1-amine (4.83 g, 31.95 mmol, 1.1 eq) and DIEA (11.26 g, 87.14 mmol, 15.18 mL, 3 eq). The reaction was stirred at 140° C. for 12 hr to give a yellow solution. LCMS showed desired MS. The reaction was diluted with EtOAc (50 mL), washed with water (50 mL*4) and brine (50 ml*2). The organic layer was dried over Na₂SO₄, concentrated in vacuum. The crude product was purified by flash column (eluting with PE/EtOAc=0% to 10%) to give 74-2a (2 g, crude).

### General Procedure for the Preparation of Compound 74-3a

To a solution of 74-2a (2 g, 6.59 mmol, 1 eq) in MeOH (70 mL) was added NaOH (6 M, 32.96 mL, 30 eq). The reaction was stirred at 70° C. for 12 hr to give a yellow suspension. LCMS showed the reaction was completed. The reaction mixture was acidified with 4N HCl(aq). to pH=5, aqueous phase extracted with EtOAc (30 ml*3). The organic layers were dried over Na₂SO₄ and concentrated to give 74-3a (1.6 g, 5.53 mmol, 83.88% yield).

### General Procedure for the Preparation of Compound 74-4a

To a solution of 74-3a (1.6 g, 5.53 mmol, 1 eq) in DMF (40 mL) was added DIEA (2.14 g, 16.59 mmol, 2.89 mL, 3 eq), HATU (2.52 g, 6.64 mmol, 1.2 eq) and DIEA (2.14 g, 16.59 mmol, 2.89 mL, 3 eq) at 0° C. The reaction was allowed to stir at 0 to 25° C. for 12 hr to black brown liquid. LCMS showed the reaction was completed. The reaction mixture was quenched with H₂O (100 mL) and extracted with MTBE (30 mL*3). The organic layers were dried over Na₂SO₄ concentrated to give the crude product. The crude product was purified by flash column (eluting with PE/EtOAc=0% to 30%) to give 74-4a (1.3 g, 2.72 mmol, 49.12% yield).

### General Procedure for the Preparation of Compound 74-5a

To a solution of 74-4a (1.8 g, 3.76 mmol, 1 eq) in MeCN (5 mL) was added POCl₃ (5.77 g, 37.61 mmol, 3.49 mL, 10 eq). The reaction was stirred at 95° C. for 2 hr to give yellow mixture. LCMS showed the reaction was completed. The reaction mixture was poured into H₂O (50 ml). The mixture was basified with 1N NaOH aq. to pH=8, extracted with EtOAc (60 mL×3). The organic layers were dried over Na₂SO₄ and concentrated to give the crude product. The crude product was purified by flash column (eluting with PE/EtOAc=0% to 10%) to give 74-5a (900 mg, crude).

### General Procedure for the Preparation of Compound 74-12

To a solution of 74-5a (900 mg, 1.95 mmol, 1 eq) in MeOH (20 mL) was added NaBH₄ (369.60 mg, 9.77 mmol, 5 eq). The reaction was stirred at 25° C. for 0.5 hr to give a yellow solution. LCMS and TLC (PE:EtOAc=3:1) showed the reaction was completed. The reaction mixture was quenched with saturated NH₄Cl (50 mL) and extracted with EtOAc (30 mL*3). The organic layers were dried over Na₂SO₄ and concentrated to give the crude product. The crude product was purified by flash column (eluting with PE/EtOAc=0% to 50%) to give 74-12 (30 mg, 64.85 μmol, 3.32% yield) and 74a-12 (470 mg, 1.02 mmol, 51.99% yield).

### General Procedure for the Preparation of Compound A-48

To a solution of 3-trimethylsilylprop-2-ynoyl chloride (15.63 mg, 97.27 μmol, 1 eq) in DCM (5 mL) was added 2-chloro-1-methyl-pyridin-1-ium; iodide (24.85 mg, 97.27 μmol, 1 eq). The reaction was stirred at 25° C. for 0.5 hr. Then the mixture was added dropwise the solution of 74-12 (45 mg, 97.27 μmol, 1 eq) and Et₃N (9.84 mg, 97.27 μmol, 13.54 μL, 1 eq) in DCM (5 mL) at 0° C. for 1 hr to give a yellow solution. LCMS showed the reaction was completed. The reaction mixture was quenched with H₂O (30 mL) and extracted with DCM (20 mL*3). The organic layers were washed with saturated NaHCO₃ (20 mL). The organic layers were dried over Na₂SO₄ and concentrated to give the crude product. The crude product was purified by prep-TLC (PE/EtOAc=3:1) to give A-48 (11 mg, 18.74 μmol, 19.27% yield).

### General Procedure for the Preparation of Compound B-84

To a solution of A-48 (45.00 mg, 76.68 μmol, 1 eq) in THF (8 mL) was added TBAF (1 M, 76.68 μL, 1 eq). The reaction was stirred at −78° C. for 0.5 hr to give a yellow solution. LCMS showed the desired MS (as a major peak). The reaction mixture was quenched with H₂O (50 mL) and extracted with EtOAc (30 mL*3). The organic layers were dried over Na₂SO₄ and concentrated to give the crude product. The crude product was purified by prep-TLC (PE/EtOAc=3:1) to give B-84 (39.32 mg, 76.40 μmol, 99.63% yield). LC-MS (m/z): 515.3 [M+H]⁺. ¹H NMR (400 MHz, CDCl₃) δ ppm 7.23-7.11 (m, 1H), 6.96-6.81 (m, 1H), 6.80-6.70 (m, 2H), 6.70-6.59 (m, 2H), 6.20-6.06 (m, 1H), 4.63-4.34 (m, 1H), 3.79-3.69 (m, 3H), 3.16-2.56 (m, 3H), 2.05-1.91 (m, 3H), 1.85-1.68 (m, 6H), 1.61-1.51 (m, 6H), 1.33-0.99 (m, 6H), 0.82-0.71 (m, 3H).

### Procedure 36: Compounds A-16 and B-81

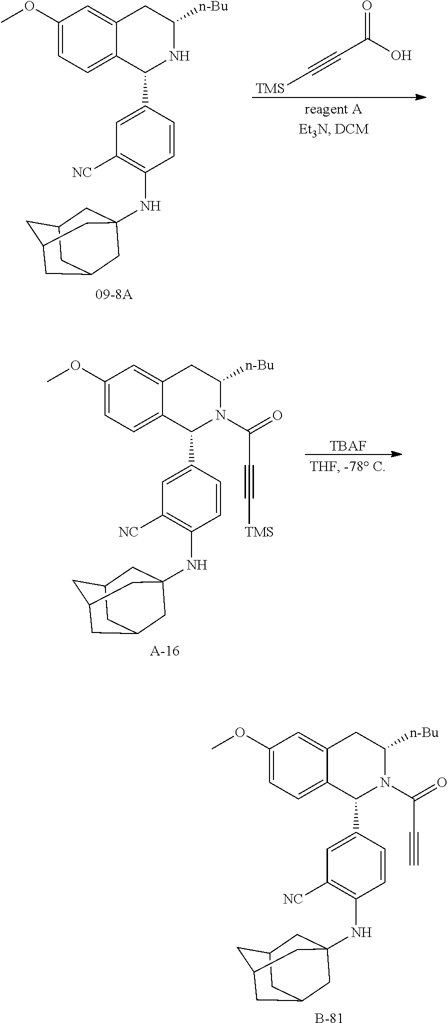

### General Procedure for the Preparation of Compound A-16

To a solution of 3-trimethylsilylprop-2-ynoic acid (32.10 mg, 225.70 μmol, 1 eq) in DCM (3 mL) was added 2-chloro-1-methyl-pyridin-1-ium; iodide (57.66 mg, 225.70 μmol, 1 eq). The reaction was stirred at 25° C. for 0.5 hr. Then the mixture was added dropwise the solution of 09-8A and Et₃N (22.84 mg, 225.70 μmol, 31.41 μL, 1 eq) in DCM (10 mL) at 0° C. The reaction was stirred at 0° C. for 1 hr to give yellow solution. LCMS and TLC (PE/EtOAc=3/1) showed the reaction was completed. The reaction mixture was quenched H₂O (10 mL) and extracted with DCM (5 mL*3). The organic layers were washed with HCl (0.5N, 5 mL), then washed with saturated NaHCO₃ (10 mL). The organic layers were dried over Na₂SO₄ and concentrated to give the crude product. The crude product was purified by column chromatography (SiO₂, Petroleum ether/Ethyl acetate=100/0 to 90/10) to give A-16 (45 mg, 75.77 μmol, 33.57% yield). LC-MS (m/z): 594.5 [M+H]⁺. ¹H NMR (400 MHz, CHLOROFORM-d) δ ppm 7.38 (dd, J=9.07, 2.06 Hz, 1H) 6.90-7.16 (m, 3H) 6.71-6.86 (m, 2H) 6.51-6.66 (m, 1H) 4.58-4.69 (m, 1H) 4.32-4.48 (m, 1H) 3.78-3.88 (m, 3H) 2.93-3.19 (m, 1H) 2.38-2.72 (m, 1H) 2.15 (br s, 3H) 1.98 (br s, 6H) 1.67-1.75 (m, 6H) 1.26 (s, 2H) 1.01-1.21 (m, 4H) 0.71-0.82 (m, 3H) 0.18-0.30 (m, 9H)

### General Procedure for the Preparation of Compound B-81

To a solution of A-16 (42 mg, 70.72 μmol, 1 eq) in THF (5 mL) were added TBAF (1 M, 77.79 μL, 1.1 eq) at −78° C. The mixture was stirred at −78° C. for 20 mins to give a white solution. LCMS and TLC (PE:EtOAc=3:1) showed the mixture was completed. The mixture was added H₂O (5 mL), extracted with EtOAc (8 mL*3). The organic layers were dried over Na₂SO₄ and concentrated to give the crude product. The residue was purified by column chromatography (SiO₂, Petroleum ether/Ethyl acetate=100/0 to 80/20) to give B-81 (19.38 mg, 22.60 μmol, 31.96% yield). LC-MS (m/z): 522.5 [M+H]⁺. ¹H NMR (400 MHz, CHLOROFORM-d) δ ppm 7.41 (br d, J=8.00 Hz, 1H) 7.08-7.22 (m, 1H) 6.44-7.06 (m, 5H) 4.18-4.85 (m, 2H) 3.84 (br d, J=6.63 Hz, 3H) 2.92-3.31 (m, 2H) 2.34-2.79 (m, 1H) 2.15 (br s, 3H) 1.91-2.05 (m, 6H) 1.70 (brs, 6H) 0.89-1.46 (m, 6H) 0.59-0.84 (m, 3H).

### Procedure 37: Compounds A-8 and B-75

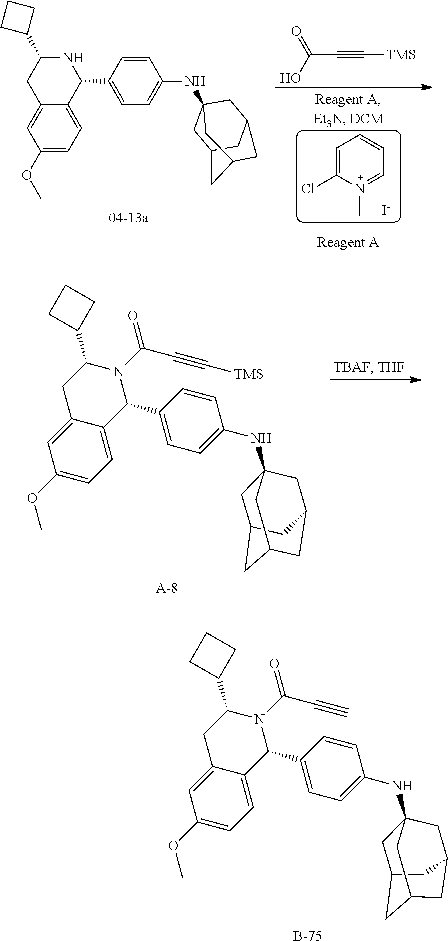

### General Procedure for the Preparation of Compound A-8

To a solution of 3-trimethylsilylprop-2-ynoic acid (22.49 mg, 158.14 μmol, 1 eq) in DCM (3 mL) was added 2-chloro-1-methyl-pyridin-1-ium; iodide (40.40 mg, 158.14 μmol, 1 eq). The mixture was stirred at 25° C. for 1 hr to give a yellow suspension. The result mixture was dropwise to a solution of 04-13a (70 mg, 158.14 μmol, 1 eq) and TEA (16.00 mg, 158.14 μmol, 22.01 μL, 1 eq) in DCM (5 mL) at 0° C. It was stirred at 0° C. for 2 hr to give a yellow solution. LCMS and TLC (PE:EtOAc=1:1, 3/1) showed the mixture was completed. The mixture was quenched with H₂O (8 mL), extracted with DCM (8 mL*3). The organic layers were washed with HCl (1N, 10 mL). The organic layer was separated. The organic layers were washed with saturated NaHCO₃ (10 mL). The organic layer was separated and dried over sodium sulfate, filtered and concentrated under vacuum. The residue was purified by column chromatography (SiO₂, Petroleum ether/Ethyl acetate=100/0 to 80/20) to give A-8 (35 mg, 60.82 μmol, 38.46% yield). LC-MS (m/z): 567.2 [M+H]⁺. ¹H NMR (400 MHz, CDCl₃) δ ppm 7.13-6.97 (m, 3H) 6.81-6.60 (m, 5H) 4.85-4.72 (m, 2H) 3.85-3.77 (m, 3H) 3.16-2.17 (m, 6H) 2.10 (br s, 3H) 1.88-1.80 (m, 6H) 1.73-1.62 (m, 8H) 0.31-0.21 (m, 9H).

### General Procedure for the Preparation of Compound B-75

To a solution of A-8 (32 mg, 56.45 μmol, 1 eq) in THF (5 mL) were added TBAF (1 M, 62.10 μL, 1.1 eq) at −78° C. The mixture was stirred at −78° C. for 15 mins to give a yellow solution. TLC (PE:EtOAc=2:1) showed the mixture was completed. The mixture was added H₂O (5 mL), extracted with EtOAc (8 mL*3). The organic layers were dried over sodium sulfate, filtered and concentrated under vacuum. The residue was purified by column chromatography (SiO₂, Petroleum ether/Ethyl acetate=100/0 to 72/28) to give B-75 (16.1 mg, 32.30 μmol, 57.22% yield). LC-MS (m/z): 495.3 [M+H]⁺. ¹H NMR (400 MHz, CDCl₃) δ ppm 7.18-6.95 (m, 3H) 6.84-6.58 (m, 5H) 4.73 (br s, 1H) 3.82 (br s, 3H) 3.31-3.17 (m, 1H) 3.13-2.83 (m, 1H) 2.75-2.47 (m, 1H) 2.40 (br s, 1H) 2.10 (br s, 3H) 1.97-1.80 (m, 8H) 1.68-1.53 (m, 8H) 1.32-1.03 (m, 1H) 0.90 (br s, 1H).

### Procedure 38: Compounds A-29 and B-76

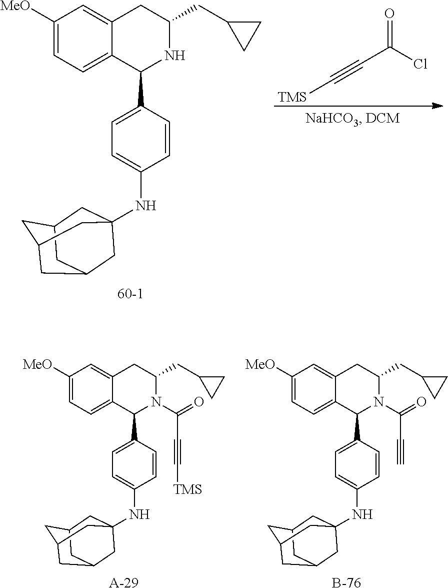

### General Procedure for the Preparation of Compound A-29 and B-76

To a solution of 60-1 (50 mg, 112.96 μmol, 1 eq) in DCM (1 mL) were added NaHCO₃ (75.92 mg, 903.68 μmol, 35.15 μL, 8 eq) and 3-trimethylsilylprop-2-ynoyl chloride (0.2 M, 1.13 mL, 2 eq). The resulting mixture was stirred at 20° C. for 12 hr to give yellow suspension. LCMS and TLC (PE/EtOAc=3:1) showed the reaction were completed. The solution was washed H₂O (10 mL) and extracted with DCM (10 mL*3). The organic layer was dried over Na₂SO₄, filtrated and concentrated. The crude product was purified by flash column (SiO₂, eluting with: PE/EtOAc=0% to 30%) to give A-29 (7.12 mg, 11.68 μmol, 10.34% yield) and B-76 (4.8 mg, 9.51 μmol, 8.42% yield).

LC-MS (m/z): 567.3 [M+H]⁺. ¹H NMR (400 MHz, CDCl₃) δ ppm 7.30 (s, 1H) 7.21 (d, J=8.53 Hz, 1H) 6.94 (dd, J=8.53, 3.26 Hz, 2H) 6.79 (td, J=8.03, 2.51 Hz, 1H) 6.73-6.61 (m, 3H) 6.20 (d, J=9.03 Hz, 1H) 4.78-4.64 (m, 1H) 3.81 (s, 3H) 3.23-2.92 (m, 2H) 2.07 (br s, 3H) 1.81 (br s, 6H) 1.57-0.77 (m, 9H) 0.66-0.54 (m, 1H) 0.53-0.34 (m, 2H) 0.28-0.08 (m, 9H) 0.08-0.05 (m, 2H).

LC-MS (m/z): 495.3 [M+H]⁺. ¹H NMR (400 MHz, CDCl₃) δ ppm 7.29 (d, J=8.28 Hz, 1H) 7.21 (d, J=8.53 Hz, 1H) 6.91 (dd, J=15.18, 8.41 Hz, 2H) 6.80 (ddd, J=15.18, 8.28, 2.64 Hz, 1H) 6.74-6.70 (m, 1H) 6.64 (dd, J=11.04, 8.53 Hz, 2H) 6.26-6.15 (m, 1H) 4.82-4.56 (m, 1H) 3.81 (d, J=5.77 Hz, 3H) 3.22-3.10 (m, 1H) 3.03-2.81 (m, 2H) 2.13-2.00 (m, 3H) 1.83 (dd, J=11.42, 2.38 Hz, 6H) 1.59-1.41 (m, 6H) 1.35-0.79 (m, 3H) 0.72-0.55 (m, 1H) 0.53-0.32 (m, 2H) 0.16-0.05 (m, 2H).

### Procedure 39: Compounds A-28 and B-77

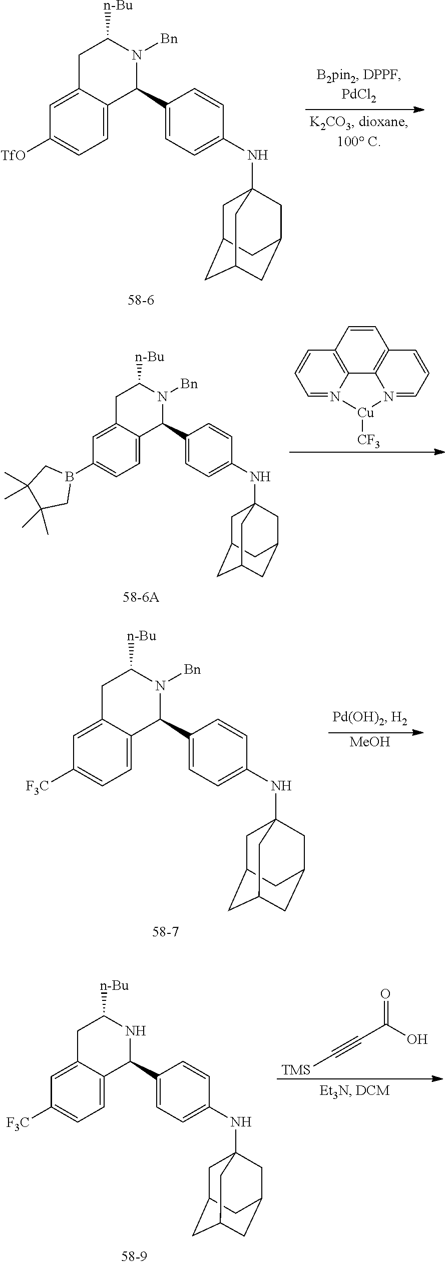

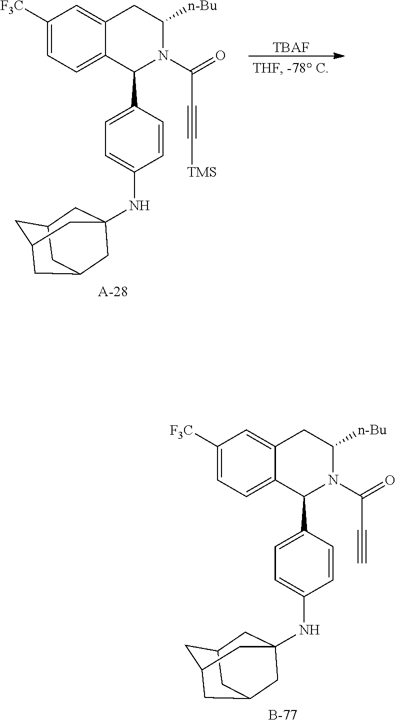

### General Procedure for the Preparation of Compound 58-6A

58-6 (0.56 g, 857.83 μmol, 1 eq), Bis(pinacolato)diboron (326.75 mg, 1.29 mmol, 1.5 eq), K₂CO₃ (252.57 mg, 2.57 mmol, 3 eq), Pd(dppf)Cl₂ (75.32 mg, 102.94 μmol, 0.12 eq) and cyclopentyl(diphenyl)phosphane; iron (71.33 mg, 128.68 μmol, 0.15 eq) were added to a 25 mL Schenlk tube at N₂ atmosphere. Then dioxane (8 mL) was injected, and the tube was sealed with parafilm. The reaction mixture was stirred for 16 hours at 100° C. LCMS and TLC (PE/EtOAc=5:1) showed 90% 58-6 was consumed. The desired MS of 58-6A (as a major peak) was found. The reaction mixture were quenched by H₂O (30 mL) and extracted by EA (50 mL*3). The combined organic layers were dried over anhydrous sodium sulfate, filtered and concentrated under reduced pressure to afford the crude product. The crude product was purified by silica column eluting with petroleum ether:EtOAc (50:1 to 30:1 to 20:1) to afford 58-6A (0.48 g, 761.05 μmol, 88.72% yield).

### General Procedure for the Preparation of Compound 58-7

A 20 mL vial was charged with 1,10-phenanthroline; trifluoromethylcopper (243.98 mg, 780.08 μmol, 1.2 eq) and KF (83.09 mg, 1.43 mmol, 33.50 μL, 2.2 eq). Then a solution of 58-6A (410 mg, 650.06 μmol, 1 eq) in DMF (15 mL) was added. The mixture was bubbled with air using air balloon for 10 mins. Then balloon was removed and the vial was stirred at 50° C. for 16 hours (open to air). LCMS and TLC (PE:EtOAc=3:1) showed the mixture was completed. The mixture was concentrated under reduced pressure to afford the crude product. The crude product was purified by silica column eluting with petroleum ether:EtOAc (50:1 to 35:1) to afford 58-7 (125 mg, 174.60 μmol, 26.86% yield).

### General Procedure for the Preparation of Compound 58-9

58-7 (110 mg, 192.06 μmol, 1 eq) and was dissolved in MeOH (4 mL) and THF (4 mL), Pd/C (10 mg, 192.06 μmol, 1 eq) was added into the reaction mixture. The system was flushed with H₂ and then evacuated and backfilled with H₂ (387.15 ug, 192.06 μmol, 1 eq) (3 times). The mixture was stirred at 20° C. for 16 h. LCMS and TLC (PE:EtOAc=1:1) showed the mixture was completed. The reaction mixture were filtered and concentrated under reduced pressure to afford the crude product. The crude product was purified by flash column on silica gel eluting with EtOAc/PE (20:1 to 1:1) to afford 58-9 (50 mg, 88.06 μmol, 45.85% yield).

### General Procedure for the Preparation of Compound A-28

To a solution of 3-trimethylsilylprop-2-ynoic acid (29.40 mg, 206.68 μmol, 1.05 eq) in DCM (3 mL) was added 2-chloro-1-methyl-pyridin-1-ium iodide (60.35 mg, 236.21 μmol, 1.2 eq), the mixture was stirred at 25° C. for 0.5 h. Then a solution of 58-9 (95 mg, 196.84 μmol, 1 eq) and TEA (23.90 mg, 236.21 μmol, 32.88 μL, 1.2 eq) in DCM (3 mL) was added dropwise at 0° C. The mixture was stirred at 0° C. for 1 h to give a yellow solution. LCMS and TLC (PE:EtOAc=2:1) showed the mixture was completed. The mixture was quenched by NH₄Cl (10 mL), the organic layer was washed by HCl (0.5 mL, pH=3˜4), and NaHCO₃ (saturated, aq., 10 mL). The organic layer was dried over anhydrous sodium sulfate and concentrated under reduced pressure to afford the crude product. The crude product was purified by flash column on silica gel eluting with EtOAc in PE (5% to 15%) and dried by lyophilized to afford A-28 (76 mg, 122.74 μmol, 62.35% yield). LC-MS (m/z): 607.1 [M+H]⁺. ¹H NMR (400 MHz, CDCl₃) δ ppm 7.48-7.58 (m, 1H), 7.36-7.46 (m, 2H), 6.87-6.99 (m, 2H), 6.65 (dd, J=19.89, 8.51 Hz, 2H), 6.32 (d, J=15.63 Hz, 1H), 4.56-4.71 (m, 1H), 3.21 (br dd, J=15.13, 4.63 Hz, 1H), 3.00 (br dd, J=15.38, 5.00 Hz, 1H), 2.81-2.91 (m, 1H), 2.03-2.15 (m, 3H), 1.83 (br s, 8H), 1.06-1.35 (m, 6H), 0.75-0.95 (m, 4H), 0.27 (s, 5H), 0-0.15 (m, 10H).

### General Procedure for the Preparation of Compound B-77

To a solution of A-28 (60 mg, 98.87 μmol, 1 eq) in THF (5 mL) was added TBAF (1 M, 118.65 μL, 1.2 eq), and then the mixture was stirred at −78° C. for 1 h. LCMS and TLC (PE:EtOAc=3:1) showed the mixture was completed. The mixture was quenched by NH₄Cl (10 mL), the organic layer was washed by NaCl (saturated, aq., 10 mL). The organic layer was dried over anhydrous sodium sulfate and concentrated under reduced pressure to afford the crude product. The crude product was purified by flash column on silica gel eluting with 15% EtOAc in petroleum ether to afford product which was dried by lyophilized to afford B-77 (20 mg, 37.41 μmol, 37.83% yield). LC-MS (m/z): 535.3 [M+H]⁺. ¹H NMR (400 MHz, CDCl₃) δ ppm 7.49-7.61 (m, 2H), 7.39-7.44 (m, 1H), 6.90 (dd, J=16.56, 8.53 Hz, 2H), 6.64 (dd, J=13.05, 8.53 Hz, 2H), 6.25-6.39 (m, 1H), 4.48-4.73 (m, 1H), 4.10 (d, J=6.78 Hz, 1H), 3.23 (br dd, J=15.31, 4.77 Hz, 1H), 3.16 (s, 1H), 2.96 (s, 1H), 2.79-2.92 (m, 1H), 2.04-2.13 (m, 3H), 1.84 (dd, J=11.17, 2.38 Hz, 6H), 1.50-1.72 (m, 12H), 1.18-1.33 (m, 5H), 1.00 (d, J=6.78 Hz, 1H), 0.80-0.88 (m, 4H).

### Procedure 40: Compounds A-27 and B-78

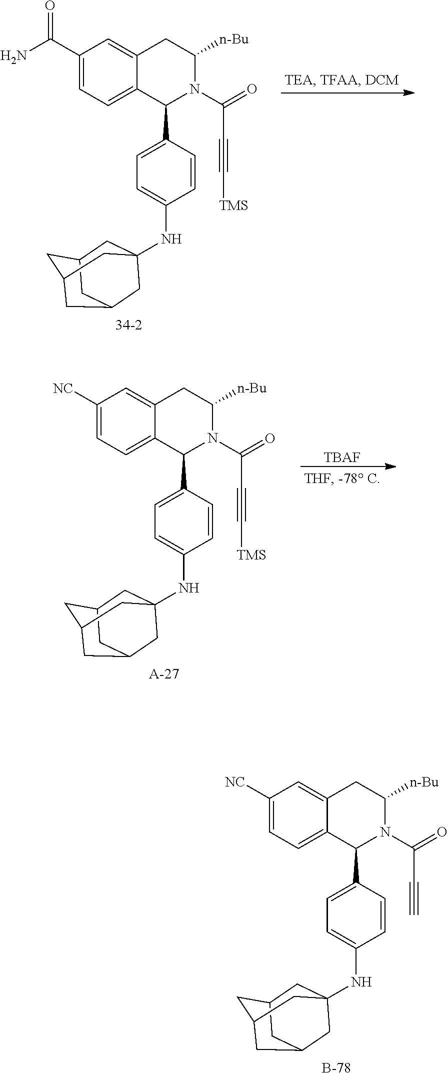

### General Procedure for the Preparation of Compound A-27

To a solution of 34-2 (15 mg, 25.78 μmol, 1 eq) in DCM (5 mL) cooled at 0° C. were added TEA (15.65 mg, 154.68 μmol, 21.53 μL, 6 eq) and TFAA (21.66 mg, 103.12 μmol, 14.34 μL, 4 eq). The mixture was stirred at 0° C. for 2 hr to give a clear solution. LCMS and TLC (PE:EtOAc=1:3) showed the mixture was completed. The mixture was quenched with H₂O (8 mL), extracted with DCM (5 mL*3). The organic layers were dried over sodium sulfate, filtered and concentrated under vacuum. Batches were combined (13 mg 34-2) for purification. The residue was purified by prep-TLC (SiO₂, Petroleum ether/Ethyl acetate=2/1) to give A-27 (15 mg).

### General Procedure for the Preparation of Compound B-78

To a solution of A-27 (15 mg, 26.60 μmol, 1 eq) in THF (3 mL) were added TBAF (1 M, 29.26 μL, 1.1 eq) at −78° C. The mixture was stirred at −78° C. for 20 mins to give a clear solution. TLC (PE:EtOAc=2:1) showed the mixture was completed. The mixture was added H₂O (8 mL), extracted with EtOAc (8 mL*3). The organic layers were dried over sodium sulfate, filtered and concentrated under vacuum. The residue was purified by prep-TLC (SiO₂, Petroleum ether/Ethyl acetate=2:1) to give B-78 (7.96 mg, 16.19 μmol, 60.86% yield). LC-MS (m/z): 492.2 [M+H]⁺. ¹H NMR (400 MHz, CDCl₃) δ ppm 7.72-7.55 (m, 1H) 7.54-7.48 (m, 1H) 7.46 (s, 1H) 7.43-7.27 (m, 1H) 7.21-7.09 (m, 1H) 7.08-6.98 (m, 1H) 6.96-6.75 (m, 1H) 6.50-6.17 (m, 1H) 4.79-4.50 (m, 1H) 3.30-2.74 (m, 3H) 2.15-1.95 (m, 3H) 1.90-1.69 (br s, 6H) 1.67-1.57 (m, 6H) 1.33-1.08 (m, 6H) 0.89-0.83 (m, 3H)

### Procedure 41: Compounds A-26, A-55, B-79, and B-80

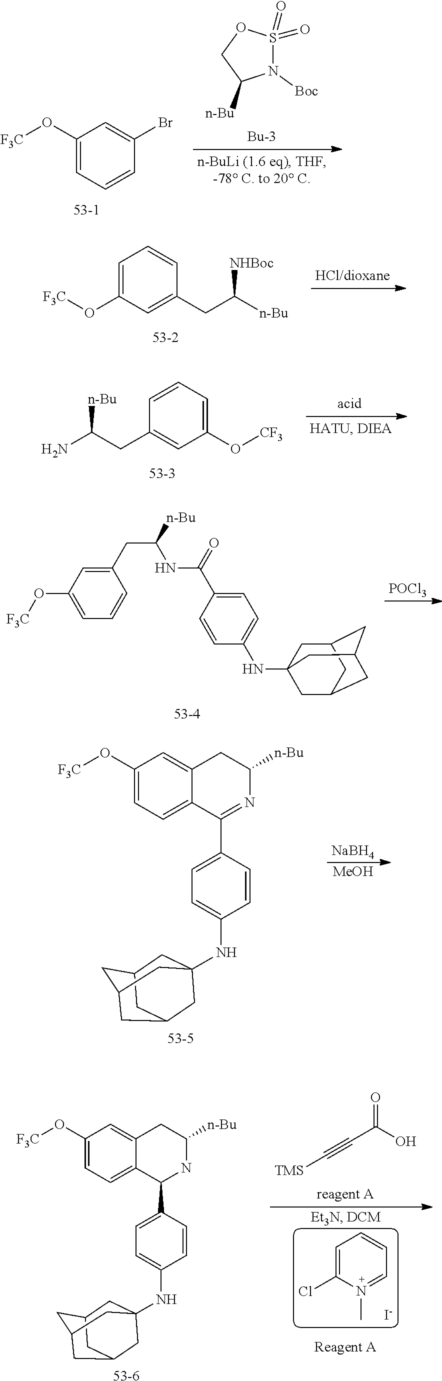

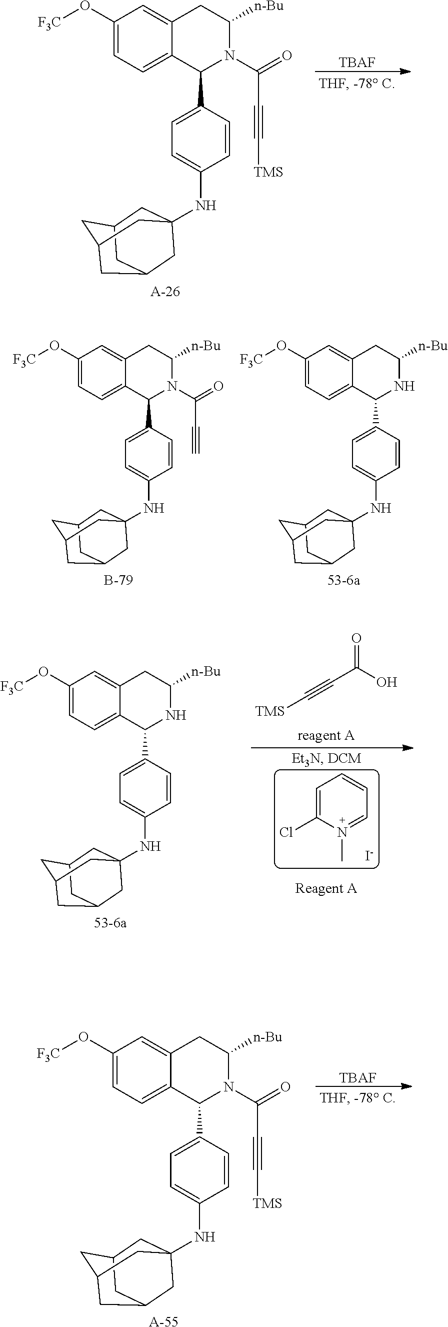

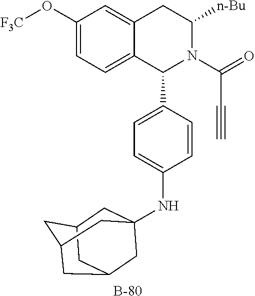

### General Procedure for the Preparation of Compound 53-2

To a solution of 53-1 (6.5 g, 26.97 mmol, 4.01 mL, 1.5 eq) in THF (50 mL) was added dropwise n-BuLi (2.5 M, 11.51 mL, 1.6 eq) at −78° C. The reaction was stirred at −78° C. for 0.5 hr. Then Bu-3 (5.02 g, 17.98 mmol, 1 eq) in THF (30 mL) was added dropwise at −78° C. The reaction was allowed to stir at 20° C. for 12 hr to give a yellow solution. LCMS and TLC (eluting with: PE/EtOAc=6/1) showed the reaction was completed. The reaction mixture was quenched with saturated citric acid (20 mL) and extracted with EtOAc (30 mL*3). The organic layers were dried over Na₂SO₄ and concentrated to give the crude product. The crude product was purified by flash column (eluting with PE/EtOAc=0% to 15%) to give 53-2 (1.6 g, 4.43 mmol, 24.62% yield). ¹H NMR (400 MHz, CDCl₃) δ ppm 7.26-7.20 (m, 1H), 7.18-7.08 (m, 1H), 7.02 (br dd, J=19.95, 7.57 Hz, 1H), 4.31-4.14 (m, 1H), 2.78-2.58 (m, 1H), 1.38-1.30 (m, 8H), 1.29 (br s, 2H), 1.27-1.23 (m, 1H), 1.21 (br dd, J=5.44, 2.06 Hz, 1H), 0.83-0.77 (m, 3H).

### General Procedure for the Preparation of Compound 53-3

To a solution of 53-2 (1.6 g, 4.43 mmol, 1 eq) was dissolved in HCl/dioxane (4 M, 1.11 mL, 1 eq) at 0° C. The reaction was allowed to stir at 25° C. for 12 hr to give a colorless oil. LCMS showed the reaction was completed. The reaction mixture was concentrated directly to give 53-3 (1.0 g, 3.83 mmol, 86.45% yield). The product was used for next further without purification. ¹H NMR (400 MHz, MeOD) δ ppm 7.53-7.47 (m, 1H), 7.47 (br s, 1H), 7.36-7.29 (m, 1H), 7.25 (br s, 2H), 3.50 (quin, J=6.69 Hz, 1H), 3.01 (d, J=7.13 Hz, 2H), 1.70-1.58 (m, 2H), 1.42-1.32 (m, 4H), 0.96-0.91 (m, 1H), 0.98-0.90 (m, 3H).

### General Procedure for the Preparation of Compound 53-4

To a solution of 4-(1-adamantylamino)benzoic acid (1.04 g, 3.83 mmol, 1 eq) in DMF (20 mL) was added DIEA (989.30 mg, 7.65 mmol, 1.33 mL, 2 eq), 53-3 (1 g, 3.83 mmol, 1 eq) and HATU (1.53 g, 4.02 mmol, 1.05 eq) at 0° C. The reaction was allowed to stir at 0 to 25° C. for 12 hr to black brown liquid. LCMS and TLC (eluting with: PE/EtOAc=3/1) showed the reaction was completed. The reaction mixture was quenched with H₂O (100 mL) and extracted with MBTE (30 ML*3). The organic layers were dried over Na₂SO₄ concentrated to give the crude product. The crude product was purified by flash column (eluting with: PE/EtOAc=0% to 20%) to give 53-4 (620 mg, 1.20 mmol, 31.48% yield). ¹H NMR (400 MHz, CDCl₃) δ ppm 7.45-7.39 (m, 2H), 7.27-7.21 (m, 1H), 7.09 (d, J=7.63 Hz, 1H), 7.03-6.97 (m, 2H), 6.65-6.59 (m, 2H), 4.33-4.23 (m, 1H), 2.86-2.77 (m, 2H), 2.06 (br s, 3H), 1.91-1.84 (m, 7H), 1.63 (br s, 6H), 1.31-1.27 (m, 2H), 1.19 (t, J=7.13 Hz, 4H), 0.81-0.76 (m, 4H).

### General Procedure for the Preparation of Compound 53-5

To solution of 53-4 (620 mg, 1.20 mmol, 1 eq) in DCM (10 mL) were added 2-chloropyridine (410.41 mg, 3.61 mmol, 342.00 μL, 3 eq) and Tf₂O (1.02 g, 3.61 mmol, 596.34 μL, 3 eq) at −78° C. The reaction was allowed to stir at 25° C. for 12 h to give a yellow solution. LCMS and TLC (eluting with: PE/EtOAc=3/1) showed the reaction was completed. The reaction mixture was quenched with saturated NaHCO₃ (20 mL) and extracted with DCM (30 mL*3). The organic layers were dried over Na₂SO₄ and concentrated to give the crude product. The crude product was purified by flash column (eluting with: PE/EtOAc=0% to 30%) to give 53-5 (420 mg, 845.74 μmol, 70.20% yield). ¹H NMR (400 MHz, CDCl₃) δ ppm 7.53-7.48 (m, 1H), 7.48-7.40 (m, 3H), 7.13-7.05 (m, 3H), 3.55-3.38 (m, 1H), 2.98-2.89 (m, 1H), 2.67-2.53 (m, 1H), 2.00-1.92 (m, 9H), 1.75-1.70 (m, 8H), 1.46-1.35 (m, 4H), 0.98-0.93 (m, 3H).

### General Procedure for the Preparation of Compound 53-6 & 53-6a

To a solution of 53-5 (170 mg, 342.32 μmol, 1 eq) in MeOH (5 mL) was added NaBH₄ (64.75 mg, 1.71 mmol, 5 eq). The reaction was stirred at 25° C. for 0.5 hr to give a yellow solution. TLC (eluting with: PE/EtOAc=2/1) showed the reaction was completed. The reaction was quenched with saturated NH₄Cl (10 mL) and extracted with EtOAc (20 mL*3). The organic layers were dried over Na₂SO₄ and concentrated to give the crude product. The crude product was purified by flash column (eluting with: PE/EtOAc=0% to 50%) to give 53-6 (30 mg, 60.17 μmol, 17.58% yield) as a yellow gum and 53-6a (60 mg, 120.33 μmol, 35.15% yield). ^{cis1}H NMR (400 MHz, CDCl₃) δ ppm 7.12-7.06 (m, 2H), 6.96-6.84 (m, 2H), 6.79-6.74 (m, 3 H), 4.95 (s, 1H), 3.08-2.82 (m, 3H), 2.13 (s, 3H), 1.90 (s, 6H), 1.72-1.66 (m, 6H), 1.44-1.35 (m, 6H), 0.96-0.92 (m, 3H).

### General Procedure for the Preparation of Compound A-26

To a solution of 3-trimethylsilylprop-2-ynoic acid (7.96 mg, 55.95 μmol, 0.9 eq) in DCM (3 mL) was added 2-chloro-1-methyl-pyridin-1-ium; iodide (14.30 mg, 55.95 μmol, 0.9 eq). The reaction was stirred at 25° C. for 0.5 hr. Then the mixture was added dropwise the solution of 53-6 (31.00 mg, 62.17 μmol, 1 eq) and Et₃N (6.29 mg, 62.17 μmol, 8.65 μL, 1 eq) in DCM (3 mL) at 0° C. LCMS showed the reaction was completed. The reaction mixture was quenched with H₂O (0.5N, 10 mL) and extracted with DCM (20 mL*3). The organic layers were washed with saturated NaHCO₃ (15 mL). The organic layers were dried over Na₂SO₄ and concentrated to give the crude product. The crude product was purified by flash column (eluting with: PE/EtOAc=0% to 30%) to give A-26 (16 mg, 25.69 μmol, 41.32% yield).

### General Procedure for the Preparation of Compound A-55

To a solution of 3-trimethylsilylprop-2-ynoic acid (26.96 mg, 189.52 μmol, 0.9 eq) in DCM (5 mL) was added 2-chloro-1-methylpyridin-1-ium; iodide (48.42 mg, 189.52 μmol, 0.9 eq). The reaction was stirred at 25° C. for 0.5 hr. Then the mixture was added dropwise the solution of 53-6a (105.00 mg, 210.58 μmol, 1 eq) and Et₃N (21.31 mg, 210.58 μmol, 29.31 μL, 1 eq) in DCM (5 mL) at 0° C. The reaction was stirred at 0° C. for 0.5 hr to give a yellow solution. LCMS showed the reaction was completed. The reaction mixture was quenched with H₂O (0.5N, 10 mL) and extracted with DCM (20 mL*3). The organic layers were washed with saturated NaHCO₃ (15 mL). The organic layers were dried over Na₂SO₄ and concentrated to give the crude product. The crude product was purified by flash column (eluting with: PE/EtOAc=0% to 30%) to give A-55 (32 mg, 51.38 μmol, 24.40% yield).

### General Procedure for the Preparation of Compound B-79

To a solution of A-26 (16 mg, 25.69 μmol, 1 eq) in THF (6 mL) was added TBAF (1 M, 25.69 μL, 1 eq). The reaction was stirred at −78° C. The reaction was stirred at −78° C. for 0.5 hr to give a yellow solution. TLC (eluting with: PE/EtOAc=3/1) showed the reaction was completed. The reaction mixture was quenched with H₂O (10 mL) and extracted with EtOAc (30 mL*3). The organic layers were dried over Na₂SO₄ and concentrated to give the crude product. The crude product was purified by flash column (eluting with: PE/EtOAc=0% to 50%) to give B-79 (5 mg, 9.08 μmol, 35.35% yield). LC-MS (m/z): 551.5[M+H]⁺. ¹H NMR (400 MHz, CDCl₃) δ ppm 7.44-7.30 (m, 1H), 7.20-7.06 (m, 1H), 7.04-6.98 (m, 1H), 6.95-6.83 (m, 2H), 6.72-6.51 (m, 2H), 6.39-6.07 (m, 1H), 4.74-4.19 (m, 1H), 3.23-2.62 (m, 3H), 2.12-2.02 (m, 3H), 1.87-1.80 (m, 6H), 1.75-1.65 (m, 6H), 1.35-1.07 (m, 6H), 0.86-0.78 (m, 3H).

### General Procedure for the Preparation of Compound B-80

To a solution of A-55 (32.00 mg, 51.38 μmol, 1 eq) in THF (12 mL) was added TBAF (1 M, 51.38 μL, 1 eq). The reaction was stirred at −78° C. The reaction was stirred at −7° C. for 0.5 hr to give a yellow solution. TLC (eluting with: PE/EtOAc=3/1) showed the reaction was completed. The reaction mixture was quenched with H₂O (10 mL) and extracted with EtOAc (30 mL*3). The organic layers were dried over Na₂SO₄ and concentrated to give the crude product. The crude product was purified by flash column (eluting with: PE/EtOAc=0% to 30%) to give B-80 (16 mg, 28.18 μmol, 54.86% yield). LC-MS (m/z): 551.5[M+H]⁺. ¹H NMR (400 MHz, CDCl₃) δ ppm 7.18-6.90 (m, 3H), 6.81 (d, J=8.25 Hz, 1H), 6.77-6.56 (m, 3H), 4.66-4.14 (m, 1H), 3.16-2.85 (m, 2H), 2.71-2.36 (m, 1H), 2.03 (br d, J=1.13 Hz, 3H), 1.79 (dd, J=6.63, 2.38 Hz, 7H), 1.63-1.53 (m, 6H), 1.29-0.79 (m, 6H), 0.77-0.60 (m, 3H).

### Procedure 42: Compound A-36

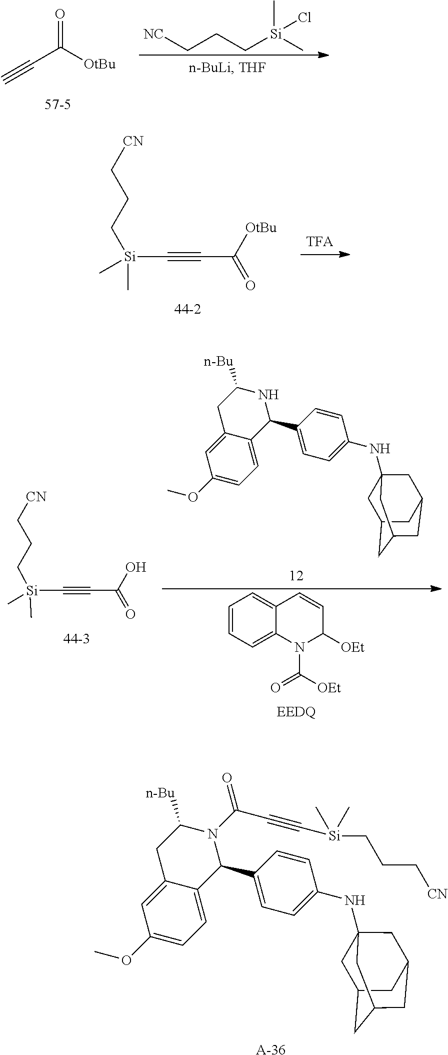

### General Procedure for the Preparation of Compound 44-2

Tert-butyl prop-2-ynoate (2.01 g, 15.94 mmol, 2.19 mL, 1 eq) was dissolved in anhydrous THF (20 mL) and the solution was cooled to −78° C. Under N2, with stirring. n-BuLi (2.5 M, 7.01 mL, 1.1 eq) was added to the above solution and the mixture was stirred for 30 min. 4-[chloro(dimethyl)silyl]butanenitrile (2.83 g, 17.53 mmol, 1.1 eq) was then added and the resulting solution was stirred at −78° C. for 1 h and allowed to warm to 20° C. over a period of 2 h. TLC (PE/EtOAc=10:1) showed the reaction was completed. The reaction was quenched with water (10 mL) was added to the crude reaction mixture. The reaction mixture was poured into a separatory funnel, the organic layer was separated and the aqueous layer was extracted with EA (4×20 mL). The combined organic layers were washed with brine (20 mL), dried over anhydrous Na₂SO₄, filtered, and concentrated in vacuo. The crude product was purified by silica column eluting with EtOAc/petroleum ether (2% to 8%) to afford 44-2 (1.6 g, 6.36 mmol, 39.94% yield).

### General Procedure for the Preparation of Compound 44-3

The mixture of 44-2 in TFA/DCM=95:5 (4.77 mmol, 12 mL, 1 eq) was stirred at 20° C. for 12 hr to give a yellow solution. TLC (PE/EtOAc=1:1) showed the reaction was completed. The reaction mixture was blow dry the solvent to concentrated with N₂ to give the crude product. The residue was purified by column chromatography (SiO₂, Petroleum ether/Ethyl acetate=100/0 to 83/17) to give 44-3 (660 mg, 3.38 mmol, 70.80% yield).

### General Procedure for the Preparation of Compound A-36

To a solution of 12 (320 mg, 719.67 μmol, 1 eq) and 44-3 (154.60 mg, 791.63 μmol, 1.1 eq) in DCM (4 mL) was degassed with N₂ for 15 min. And then EEDQ (266.95 mg, 1.08 mmol, 1.5 eq) was added. The reaction was stirred at 20° C. for 12 h to give a yellow solution. LCMS showed the reaction was completed. The mixture was concentrated to give the crude product. The residue was purified by prep-HPLC (HCl condition; column: Welch Xtimate C18 100*40 mm*3 μm; mobile phase: [water (10 mM HCl)-ACN]; B %: 40%-70%, 8.5 min) to give A-36 (60.02 mg, 92.98 μmol, 12.92% yield). LC-MS (m/z): 622.3[M+H]⁺. ¹H NMR (CDCl₃, 400 MHz): δ=10.88 (br s, 1H), 7.51-7.38 (m, 2H), 7.35 (d, J=8.3 Hz, 1H), 7.25-7.18 (m, 2H), 6.84-6.76 (m, 1H), 6.67 (s, 1H), 6.31-6.21 (m, 1H), 4.61 (br s, 1H), 3.80 (s, 3H), 3.05-2.66 (m, 2H), 2.50-2.31 (m, 2H), 1.79 (br s, 5H), 1.59 (br s, 6H), 1.48-1.37 (m, 6H), 1.33-1.14 (m, 6H), 0.96-0.78 (m, 5H), 0.33-0.02 ppm (m, 6H).

### Procedure 43: Compound A-57

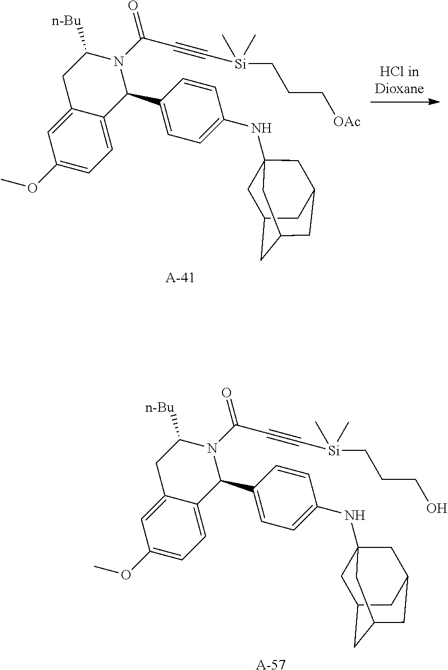

### General Procedure for the Preparation of A-57

A-41 (0.4 g, 610.73 μmol, 1 eq) was added HCl/Dioxane (4 M, 15 mL, 98.24 eq) under N2. The reaction was stirred at 20° C. for 36 hours. LCMS showed the reaction was completed. The reaction mixture was concentrated under reduced pressure. The crude product was purified by reversed-phase HPLC (HCl condition) to give A-57. LC-MS (m/z): 635.4 [M+H]⁺. ¹H NMR (400 MHz, CDCl₃) δ ppm 10.74-11.03 (m, 2H), 7.40-7.51 (m, 2H), 7.36 (d, J=8.28 Hz, 1H), 7.29 (br d, J=8.53 Hz, 1H), 7.24 (br s, 1H), 6.76-6.85 (m, 1H), 6.65-6.71 (m, 1H), 6.21-6.33 (m, 1H), 4.60-4.68 (m, 1H), 3.81 (d, J=2.01 Hz, 3H), 3.67 (t, J=6.53 Hz, 1H), 3.43 (t, J=6.90 Hz, 1H), 2.81-3.04 (m, 1H), 2.70-2.80 (m, 1H), 1.74-1.90 (m, 19H), 1.65-1.73 (m, 3H), 1.39-1.52 (m, 7H), 1.22-1.33 (m, 5H), 0.73-0.89 (m, 5H), 0.39-0.47 (m, 1H), 0.28 (s, 4H), 0.06 (d, J=1.76 Hz, 2H).

### Procedure 44: Compound B-53

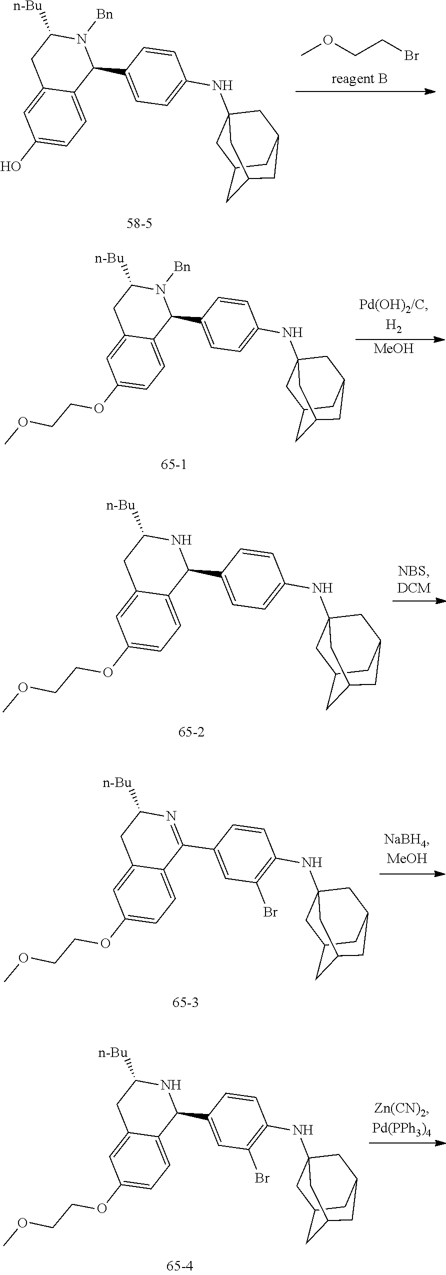

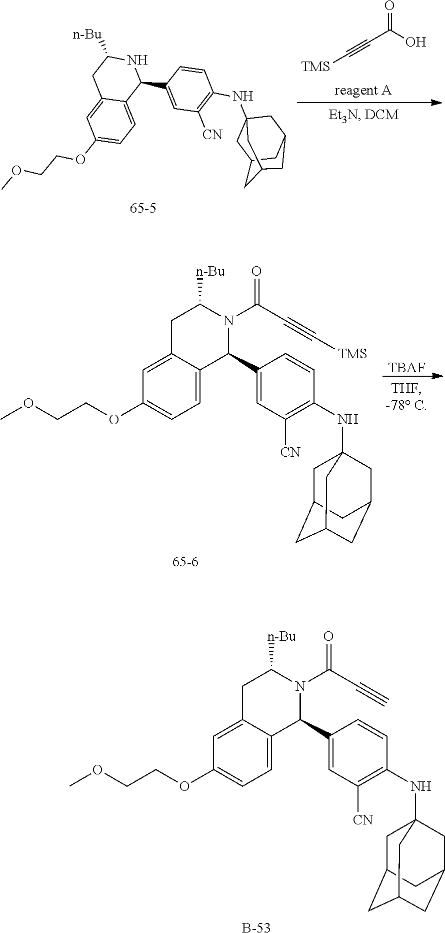

### General Procedure for the Preparation of Compound 65-1

To a solution of 58-5 (2 g, 3.84 mmol, 1 eq) in DMF (20 mL) was added 1-bromo-2-methoxy-ethane (1.28 g, 9.22 mmol, 865.65 μL, 2.4 eq), K₂CO₃ (2.12 g, 15.36 mmol, 4 eq) and KI (127.51 mg, 768.13 μmol, 0.2 eq). The reaction was stirred at 50° C. for 16 hr to give yellow solution. TLC (PE:EtOAc=3:1) showed the reaction was completed. The reaction mixture was poured into H₂O (20 mL) and extracted with EtOAc (20 mL×3). The combined organic layers were dried over Na₂SO₄, filtered and concentrated under reduced pressure. The crude product was purified by flash column (eluting with: PE/EtOAc=0% to 7%) to give 65-1 (1.6 g, 2.76 mmol, 71.97% yield).

### General Procedure for the Preparation of Compound 65-2

To a solution of 65-1 (1.6 g, 2.76 mmol, 1 eq) in MeOH (20 mL) was added Pd(OH)₂ (388.19 mg, 276.42 μmol, 0.1 eq) under N₂. The suspension was degassed under vacuum and purged with H₂ several times. The reaction was stirred at 25° C. for 12 hr to give a black suspension. TLC (PE/EtOAc=3:1) showed the reaction was completed. The crude products were combined for work up. The reaction mixture was filtered on celite and washed with MeOH (30 mL). The filtrate was concentrated to give the crude product. The crude products were combined for further purification. The crude product was purified by flash column (eluting with: PE/EtOAc=0% to 35%) to give 65-2 (1.3 g).

### General Procedure for the Preparation of Compound 65-3

To a solution of 65-2 (1.30 g, 2.66 mmol, 1 eq) in DCM (14 mL) was added NBS (1.42 g, 7.98 mmol, 3 eq) at 25° C. The reaction was stirred at 25° C. for 14 hr to give a yellow solution. LCMS and TLC (PE/EtOAc=0:1) showed the reaction were completed. The reaction mixture was concentrated directly. The residue was purified by column chromatography (SiO₂, Petroleum ether/Ethyl acetate/TEA=100/0/o 35/65/0.1%) to give 65-3 (1.1 g, 1.71 mmol, 64.34% yield).

### General Procedure for the Preparation of Compound 65-4

To a solution of 65-3 (1.1 g, 1.94 mmol, 1 eq) in MeOH (12 mL) was added NaBH₄ (367.88 mg, 9.72 mmol, 5 eq). The reaction was stirred at 25° C. for 0.5 hr to give yellow solution. TLC (PE/EtOAC=1:1) showed the reaction was completed. The reaction mixture was poured into saturated NaHCO₃ aq. (15 mL) and extracted with EtOAc (15 mL×3). The combined organic layers were washed with saturated NaCl aq. and dried over Na₂SO₄, filtered and concentrated under reduced pressure. The crude product was purified by flash column (eluting with: PE/EtOAc=0% to 30%) to give 65-4 (180 mg, 317.13 μmol, 16.31% yield).

### General Procedure for the Preparation of Compound 65-5

To a solution of 65-4 (180 mg, 317.13 μmol, 1 eq), Zn(CN)₂ (122.89 mg, 1.05 mmol, 66.43 μL, 3.3 eq) and Pd(PPh₃)₄ (120.93 mg, 104.65 μmol, 0.33 eq) were taken up into a microwave tube in DMF (4 mL) under N₂. The sealed tube was heated at 120° C. for 1 hr under microwave to give a yellow suspension. LCMS showed the reaction was completed. The reaction mixture was poured into H₂O (10 mL) and extracted with EtOAc (10 mL×3). The combined organic layers were dried over Na₂SO₄, filtered and concentrated under reduced pressure. The crude product was purified by flash column (eluting with: PE/EtOAc=0% to 30%) to give 65-5 (70 mg, 97.43 μmol, 30.72% yield).

### General Procedure for the Preparation of Compound 65-6

To a solution of 3-trimethylsilylprop-2-ynoic acid (23.26 mg, 163.52 μmol, 1.2 eq) in DCM (1 mL) were added 2-chloro-1-methyl-pyridin-1-ium; iodide (34.81 mg, 136.26 μmol, 1 eq). The mixture was stirred at 20° C. for 1 hr to give a yellow suspension. Then a solution of 65-5 (70 mg, 136.26 μmol, 1 eq) and TEA (13.79 mg, 136.26 μmol, 18.97 μL, 1 eq) in DCM (1 mL) was dropwise. It was stirred at 0° C. for 1 hr to give a yellow solution. LCMS showed the reaction was completed. The reaction was basified with saturated NH₄Cl aq. (5 mL), extracted with DCM (5 mL×3). The combined organic layers were dried over Na₂SO₄, filtered and concentrated under reduced pressure. The residue was purified by prep-TLC (PE:EtOAc=3:1) to give 65-6 (20 mg, 31.35 μmol, 23.01% yield).

### General Procedure for the Preparation of Compound B-53

To a solution of 65-6 (20 mg, 31.35 μmol, 1 eq) in THF (1 mL) was added TBAF (1 M, 34.49 μL, 1.1 eq). The reaction was allowed to stir at −78° C. for 0.5 hr to give a yellow solution. TLC (PE:EtOAc=3:1) showed the reaction was completed. The reaction was basified with H₂O (5 mL), extracted with DCM (5 mL×3). The combined organic layers were dried over Na₂SO₄, filtered and concentrated under reduced pressure. The residue was purified by prep-TLC (PE:EtOAc=3:1) to give B-53 (6.59 mg, 11.65 μmol, 37.15% yield). LC-MS (m/z): 566.3 [M+H]⁺. ¹H NMR (400 MHz, CHLOROFORM-d) δ ppm 7.25-7.05 (m, 3H) 6.98-6.74 (m, 3H) 6.19-6.06 (m, 1H) 4.65-4.45 (m, 1H) 4.18-4.07 (m, 2H) 3.82-3.70 (m, 2H) 3.50-3.43 (m, 3H) 3.18-2.97 (m, 2H) 2.80-2.70 (m, 1H) 2.12 (br s, 3H) 1.97-1.92 (m, 6H) 1.68 (br s, 6H) 0.89-0.83 (m, 9H).

### Procedure 45: Compound B-54

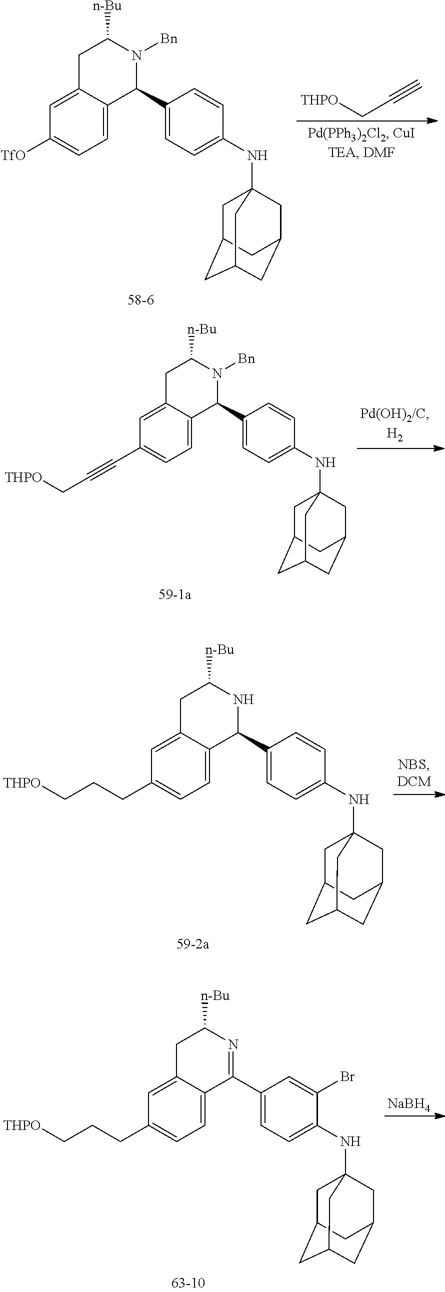

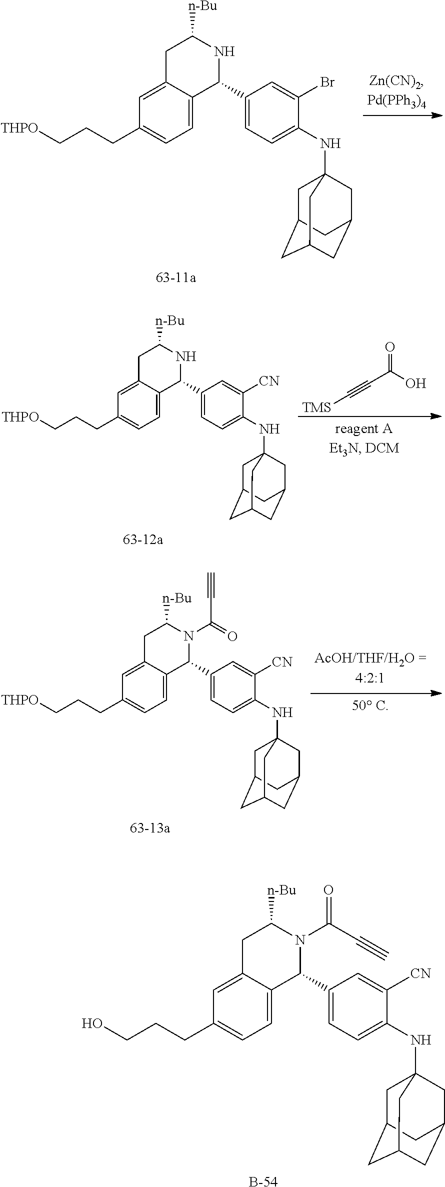

### General Procedure for the Preparation of Compound 59-1a

To a solution of 58-5 (2 g, 3.06 mmol, 1 eq) in DMF (20 mL) was added 2-prop-2-ynoxytetrahydropyran (858.93 mg, 6.13 mmol, 861.52 μL, 2 eq), TEA (930.04 mg, 9.19 mmol, 1.28 mL, 3 eq), CuI (58.35 mg, 306.37 μmol, 0.1 eq) and palladium triphenylphosphane (354.03 mg, 306.37 μmol, 0.1 eq) under N₂. The mixture was stirred at 100° C. for 12 hr to give a black suspension. LCMS and TLC (PE:EtOAc=5:1) showed the reaction were completed. The reaction mixture was diluted with H₂O (20 mL) and extracted with EtOAc (20 mL×3). The combined organic layers were washed with brine (10 mL), dried over Na₂SO₄, filtered and concentrated under reduced pressure. The crude product was purified by flash column (eluting with: PE/EtOAc=0% to 5%) to give 59-1a (1.4 g, 2.18 mmol, 71.08% yield).

### General Procedure for the Preparation of Compound 59-2a

To a solution of 59-1a (1 g, 1.56 mmol, 1 eq) in MeOH (30 mL) and THF (30 mL), H₂ (3.17 mg, 1.56 mmol, 1 eq) was added into the reaction mixture. The system was flushed with H2 and then evacuated and backfilled with Pd(OH)₂ (218.44 mg, 155.54 μmol, 0.1 eq). The mixture was stirred at 25° C. for 6 h to give a black suspension. LCMS showed the reaction was completed. The reaction mixture was filtered on celite and washed with MeOH (50 mL). The filtrate was concentrated to give the crude product. The crude product was purified by flash column (eluting with: PE/EtOAc=0% to 35%) to give 59-2a (400 mg, 718.37 μmol, 46.18% yield).

### General Procedure for the Preparation of Compound 63-10

To a solution of 59-2a (578 mg, 1.04 mmol, 1 eq) in DCM (10 mL) was added NBS (406.46 mg, 2.28 mmol, 2.2 eq) at 25° C. The reaction was stirred at 25° C. for 12 hr to give a yellow solution. LCMS showed the reaction was completed, but by-products was found. The reaction was added Na2CO3 (110.02 mg, 1.04 mmol, 1 eq). The reaction was stirred at 25° C. for 12 hr to give a yellow solution. LCMS showed the reaction was completed, but by-products was found. The reaction mixture were concentrated under reduced pressure to afford the crude product. The crude product was purified by flash column (PE:EtOAc:TEA (100/0/0 to 70/30/0.01) to give 63-10 (160 mg, 252.49 μmol, 24.32% yield).

### General Procedure for the Preparation of Compound 63-11a

To a solution of 63-10 (160 mg, 252.49 μmol, 1 eq) in MeOH (5 mL) was added NaBH₄ (28.66 mg, 757.46 μmol, 3 eq) at 0° C. The reaction was stirred at 0° C. for 1 h. LCMS showed the reaction was completed. The reaction mixture was poured into H2O (10 mL) and extracted with EtOAc (10 mL×3). The combined organic layers were dried over Na₂SO₄, filtered and concentrated under reduced pressure. The crude product was purified by flash column (eluting with: PE/EtOAc=0% to 30%) to give 63-11a (100 mg, 157.30 μmol, 62.30% yield).

### General Procedure for the Preparation of Compound 63-12a

To a solution of 63-11a (100 mg, 157.30 μmol, 1 eq), Zn(CN)₂ (55.41 mg, 471.91 μmol, 29.95 μL, 3 eq) and Pd(PPh₃)₄ (54.53 mg, 47.19 μmol, 0.3 eq) were taken up into a microwave tube in DMF (2 mL) under N₂ The sealed tube was heated at 120° C. for 1 hr under microwave to give a yellow suspension. LCMS showed the reaction was not completed, a little of R1 remained. The reaction mixture was poured into H₂O (5 mL) and extracted with EtOAc (5 mL×3). The combined organic layers were dried over Na₂SO₄, filtered and concentrated under reduced pressure. The crude product was purified by flash column (eluting with: PE/EtOAc=0% to 15%) to give 63-12a (65 mg, 87.70 μmol, 55.75% yield).

### General Procedure for the Preparation of Compound 63-13a

3-trimethylsilylprop-2-ynoic acid (19.07 mg, 134.06 μmol, 1.2 eq) in DCM (1 mL) were added 2-chloro-1-methyl-pyridin-1-ium; iodide (28.54 mg, 111.72 μmol, 1 eq). The mixture was stirred at 25° C. for 0.5 hr to give a yellow suspension. Then a solution of 63-12 (65 mg, 111.72 μmol, 1 eq) and TEA (11.30 mg, 111.72 μmol, 15.55 μL, 1 eq) in DCM (1 mL) was dropwise. It was stirred at 0° C. for 1 hr to give a yellow solution. LCMS showed the reaction was not completed, a little of R1 remained. The reaction was basified with saturated NH₄Cl aq. (5 mL), extracted with DCM (5 mL×3). The combined organic layers were dried over Na₂SO₄, filtered and concentrated under reduced pressure. The residue was purified by prep-TLC (PE:EtOAc=3:1) to give 63-13 (20 mg, 31.55 μmol, 28.24% yield).

### General Procedure for the Preparation of Compound B-54

To a solution of 63-13a (20 mg, 31.55 μmol, 1 eq) in AcOH (1 mL), THF (0.5 mL) and H₂O (0.25 mL). The reaction was stirred at 50° C. for 3 hr to give a light yellow solution. LCMS showed the reaction was not completed, a little of R1 remained. The reaction was stirred at 50° C. for 4 hr to give a light yellow solution. TLC (PE/EtOAc=3:1) showed the reaction was completed. The reaction mixture was poured into saturated NaHCO₃ aq. (10 mL) and extracted with EtOAc (10 mL×4). The combined organic layers were dried over Na₂SO₄, filtered and concentrated under reduced pressure/in vacuo. The residue was purified by prep-TLC (PE:EtOAc=1.5:1) to give B-54 (9.01 mg, 16.32 μmol, 51.74% yield). LC-MS (m/z): 550.2 [M+H]⁺. ¹H NMR (400 MHz, chloroform-d) δ ppm 7.41 (dd, J=9.03, 2.26 Hz, 1H) 7.25 (br d, J=2.01 Hz, 1H) 7.19-6.91 (m, 5H) 6.70-6.52 (m, 1H) 4.77-4.61 (m, 1H) 4.51-4.24 (m, 1H) 3.72 (td, J=6.34, 2.89 Hz, 2H) 3.23-2.92 (m, 2H) 2.80-2.66 (m, 3H) 2.46-2.22 (m, 1H) 2.15 (br s, 3H) 1.98 (br s, 6H) 1.76-1.69 (m, 6H) 1.21-0.96 (m, 6H) 0.81-0.67 (m, 3H).

### Procedure 46: Compound B-55

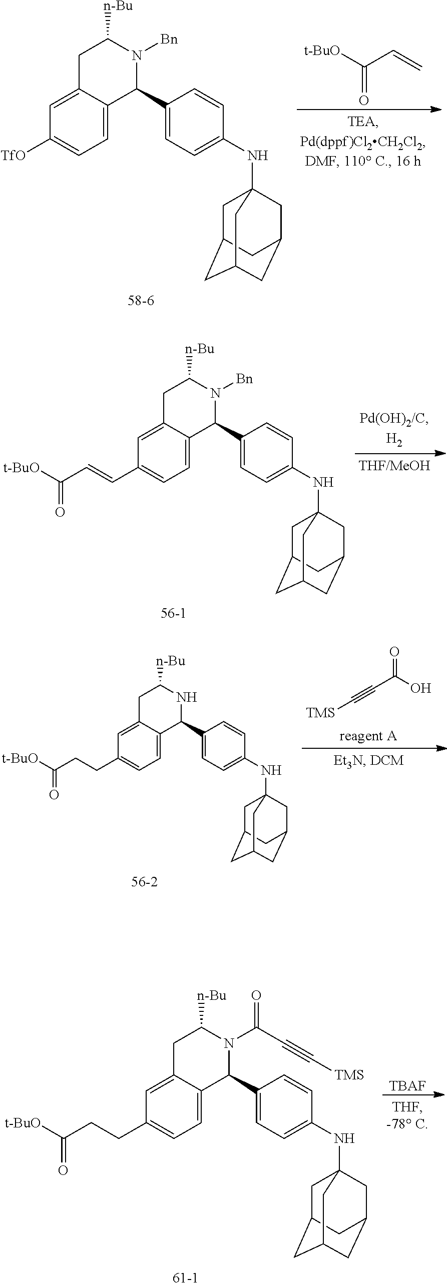

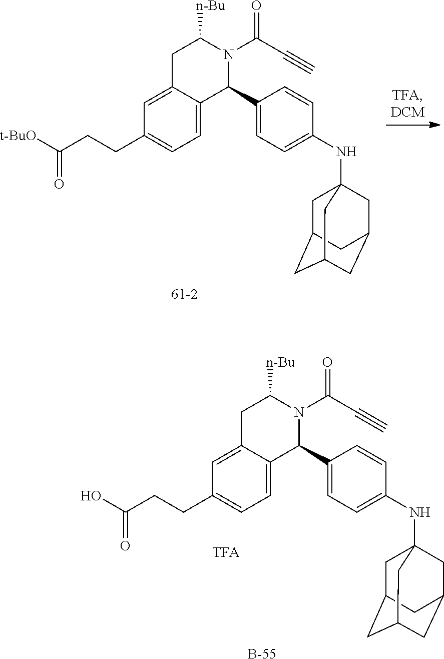

### General Procedure for the Preparation of Compound 56-1

To a solution of 58-6 (50 mg, 76.59 μmol, 1 eq) in DMF (3 mL) was added tert-butyl prop-2-enoate (98.17 mg, 765.92 μmol, 111.17 μL, 10 eq), TEA (15.50 mg, 153.18 μmol, 21.32 μL, 2 eq), Pd(dppf)Cl₂·CH₂Cl₂ (12.51 mg, 15.32 μmol, 0.2 eq) at 20° C. under N. The mixture was stirred at 110° C. for 12 hr to give a black suspension. LCMS and TLC (PE:EtOAc=5:1) showed a little 58-6 remained and desired mass was found. The mixture was added H₂s (8 mL) and filtered through celite. The filtrate was extracted with EtOAc (8 mL*3). The organic layers were dried over sodium sulfate, filtered and concentrated under vacuum. The residue was purified by prep-TLC (SiO₂, Petroleum ether/Ethyl acetate=5:1) to give 56-1 (10 mg, 15.00 μmol, 19.58% yield).

### General Procedure for the Preparation of Compound 56-2

To a solution of 56-1 (30 mg, 47.55 μmol, 1 eq) in MeOH (3 mL)/THF (1 mL) was added Pd(OH)₂/C (20 mg, 14.24 μmol, 2.99 e-1 eq) under N₂. The suspension was degassed under vacuum and purged with H₂ (164.76 ug, 81.74 μmol) several times. The mixture was stirred under H₂ (15 psi) at 20° C. for 12 hours to give a black suspension. LCMS and TLC (PE:EtOAc=5:1) showed the mixture was completed. The mixture was filtered through Celite. The filtrated was concentrated under vacuum. The crude products were combined for work up (46 mg 56-1). The residue was purified by column chromatography (SiO₂, Petroleum ether/Ethyl acetate=100/0 to 55/45) to give 56-2 (14 mg, 25.79 μmol).

### General Procedure for the Preparation of Compound 61-1

To a solution of 3-trimethylsilylprop-2-ynoic acid (22.27 mg, 156.60 μmol, 1 eq) in DCM (2 mL) was added 2-chloro-1-methyl-pyridin-1-ium; iodide (40.01 mg, 156.60 μmol, 1 eq). The mixture was stirred at 20° C. for 1 hr to give a yellow suspension. The result mixture was dropwise to a solution of 56-2 (85 mg, 156.60 μmol, 1 eq) and TEA (15.85 mg, 156.60 μmol, 21.80 μL, 1 eq) in DCM (2 mL) at 0° C. It was stirred at 0° C. for 1 hr to give a yellow solution. LCMS and TLC (PE:EtOAc=3:1) showed a little 56-2 remained and desired mass was found. The mixture was quenched with aq·NH₄Cl (10 mL), extracted with DCM (10 mL*3). The organic layer was separated and dried over sodium sulfate, filtered and concentrated under vacuum. The residue was purified by column chromatography (SiO₂, Petroleum ether/Ethyl acetate=100/0 to 83/17) to give 61-1 (97 mg, 144.83 μmol, 92.49% yield).

### General Procedure for the Preparation of Compound 61-2

To a solution of 61-1 (97 mg, 145.43 μmol, 1 eq) in THF (3 mL) were added TBAF (1 M, 159.97 μL, 1.1 eq) at −78° C. The mixture was stirred at −78° C. for 10 mins to give a clear solution. TLC (PE:EtOAc=2:1) showed the mixture was completed. The mixture was added H₂O (10 mL), extracted with EtOAc (10 mL*3). The organic layers were dried over sodium sulfate, filtered and concentrated under vacuum. The residue was purified by column chromatography (SiO₂, Petroleum ether/Ethyl acetate=100/0 to 78/22) to give 61-2 (52 mg, 87.42 μmol, 60.11% yield).

### General Procedure for the Preparation of Compound B-55

To a solution of 61-2 (42 mg, 70.61 μmol, 1 eq) in DCM (2 mL) was added TFA (3.08 g, 27.01 mmol, 2.00 mL, 382.56 eq). The mixture was stirred at 20° C. for 1.5 hr to give a yellow solution. LCMS showed the mixture was completed. The mixture was concentrated under vacuum. The crude products were combined for purification. The residue was lyophilized to give B-55 (53.64 mg, TFA). LC-MS (m/z): 539.4 [M+H]⁺. ¹H NMR (400 MHz, MeOD) δ ppm 7.58-7.47 (m, 2H) 7.42-7.05 (m, 5H) 6.57-6.11 (m, 1H) 4.82-4.46 (m, 1H) 4.13 (s, 1H) 3.30-3.20 (m, 1H) 3.04-2.93 (m, 1H) 2.89 (br t, J=7.6 Hz, 2H) 2.66-2.50 (m, 2H) 2.19 (br s, 3H) 1.86 (br s, 6H) 1.78-1.58 (m, 6H) 1.39-1.14 (m, 6H) 0.87 (brt, J=7.2 Hz, 3H).

### Procedure 47: Compound B-56

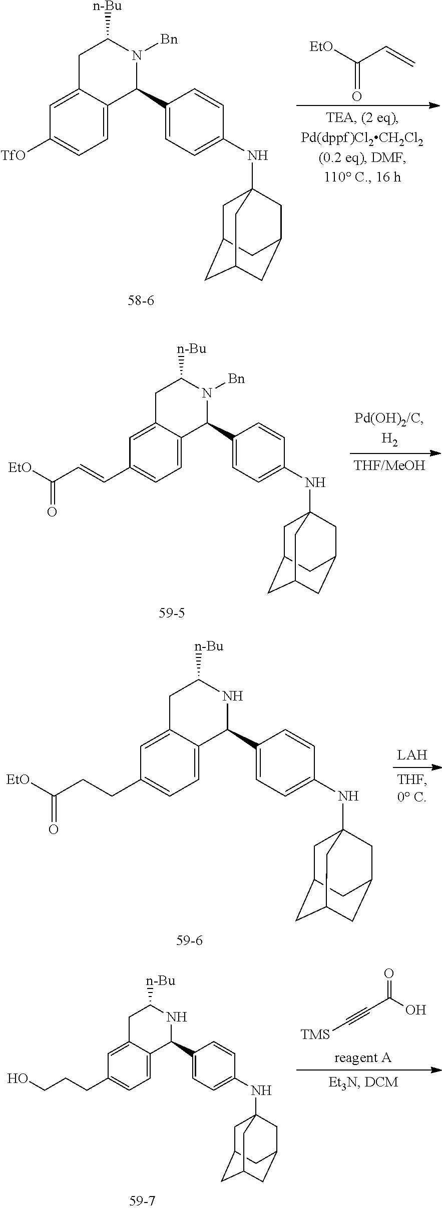

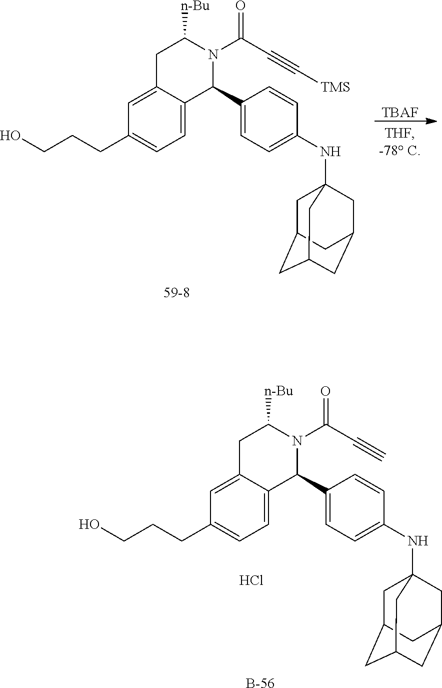

### General Procedure for the Preparation of Compound 59-5

To a solution of 58-6 (500 mg, 765.92 μmol, 1 eq) in DMF (8 mL) was added ethyl prop-2-enoate (766.81 mg, 7.66 mmol, 832.58 μL, 10 eq), TEA (155.01 mg, 1.53 mmol, 213.22 μL, 2 eq), Pd(dppf)Cl₂·CH₂Cl₂ (125.10 mg, 153.18 μmol, 0.2 eq) under N₂. The mixture was stirred at 110° C. for 12 hr to give a black suspension. TLC (PE:EtOAc=6:1) showed a little 58-6 remained and desired spot was found. The crude products were combined for work up. The mixture was concentrated under vacuum. The residue was purified by column chromatography (SiO₂, Petroleum ether/Ethyl acetate=100/0 to 94/6) to give 59-5 (216 mg, 342.77 μmol, 44.75% yield).

### General Procedure for the Preparation of Compound 59-6

To a solution of 59-5 (186 mg, 308.54 μmol, 1 eq) in MeOH (8 mL)/THF (2 mL) was added Pd(OH)₂/C (100.00 mg, 71.21 μmol, 2.31 e-1 eq) under N₂. The suspension was degassed under vacuum and purged with H₂ (164.76 ug, 81.74 μmol) several times. The mixture was stirred under H₂ (15 psi) at 20° C. for 12 hours to give a black suspension. LCMS and TLC (PE:EtOAc=4:1) showed the mixture was completed. The mixture was filtered through Celite. The filtrated was concentrated under vacuum. The crude products were combined for purification. The residue was purified by column chromatography (SiO₂, Petroleum ether/Ethyl acetate=100/0 to 64/36) to give 59-6 (98 mg, 190.39 μmol, 61.71% yield).

### General Procedure for the Preparation of Compound 59-7

To a solution of 59-6 (76 mg, 147.65 μmol, 1 eq) in THF (5 mL) was added LiAlH₄ (16.81 mg, 442.94 μmol, 3 eq) at 0° C. under N₂. The mixture was stirred at 0° C. for 2 hr to give a yellow suspension. LCMS showed the mixture was completed. The reaction was quenched with HCl (aq, 1N, 5 mL), extracted with EtOAc (5 mL*3). The aqueous layer was adjusted to pH=7 with saturated NaHCO₃. It was extracted with DCM (8 mL). The organic layers were dried over sodium sulfate, filtered and concentrated under vacuum. 59-7 (30 mg, 60.21 μmol, 40.78% yield). It was used next step without further purification. LC-MS (m/z): 473.3 [M+H]⁺.

### General Procedure for the Preparation of Compound 59-8

To a solution of 3-trimethylsilylprop-2-ynoic acid (9.03 mg, 63.46 μmol, 1 eq) in DCM (2 mL) was added 2-chloro-1-methyl-pyridin-1-ium; iodide (16.21 mg, 63.46 μmol, 1 eq). The mixture was stirred at 20° C. for 1 hr to give a yellow suspension. The result mixture was dropwise to a solution of 59-7 (30 mg, 63.46 μmol, 1 eq) and TEA (6.42 mg, 63.46 μmol, 8.83 μL, 1 eq) in DCM (2 mL) at 0° C. It was stirred at 0° C. for 1 hr to give a yellow solution. LCMS and TLC (PE:EtOAc=1:1) showed the mixture was completed. The mixture was quenched with aq·NH₄Cl (10 mL), extracted with DCM (10 mL*3). The organic layer was separated and dried over sodium sulfate, filtered and concentrated under vacuum. 59-8 (56 mg, crude) was used next step without further purification. LC-MS (m/z): 597.2 [M+H]⁺

### General Procedure for the Preparation of Compound B-56

To a solution of 59-8 (56 mg, 70.01 μmol, 1 eq) in THF (3 mL) were added TBAF (1 M, 77.01 μL, 1.1 eq) at −78° C. The mixture was stirred at −78° C. for 10 mins to give a clear solution. LCMS and TLC (PE:EtOAc=2:3) showed the mixture was completed. The mixture was quenched with H₂O (8 mL), extracted with EtOAc (8 mL*3). The organic layers were dried over sodium sulfate, filtered and concentrated under vacuum. The residue was purified by prep-TLC (SiO₂, Petroleum ether/Ethyl acetate=1:1) to give impure product (15 mg). The residue was purified by prep-HPLC (HCl condition; column: 3_Phenomenex Luna C18 75*30 mm*3 μm; mobile phase: [water (0.05% HCl)-ACN]; B %: 15%-45%, 8.5 min) to give B-56. LC-MS (m/z): 525.1 [M+H]⁺. ¹H NMR (400 MHz, DMSO-d₆) δ ppm 10.61 (br s, 1H) 7.63-7.32 (m, 3H) 7.27-7.11 (m, 2H) 7.10-6.98 (m, 2H) 6.44-6.00 (m, 1H) 4.81-4.30 (m, 2H) 3.18-3.05 (m, 1H) 2.93-2.78 (m, 2H) 2.60-2.55 (m, 2H) 2.36-2.30 (m, 1H) 2.10 (br s, 3H) 1.78 (br s, 6H) 1.71-1.47 (m, 10H) 1.30-1.08 (m, 6H) 0.85-0.76 (m, 3H).

### Procedure 48: Compound B-57

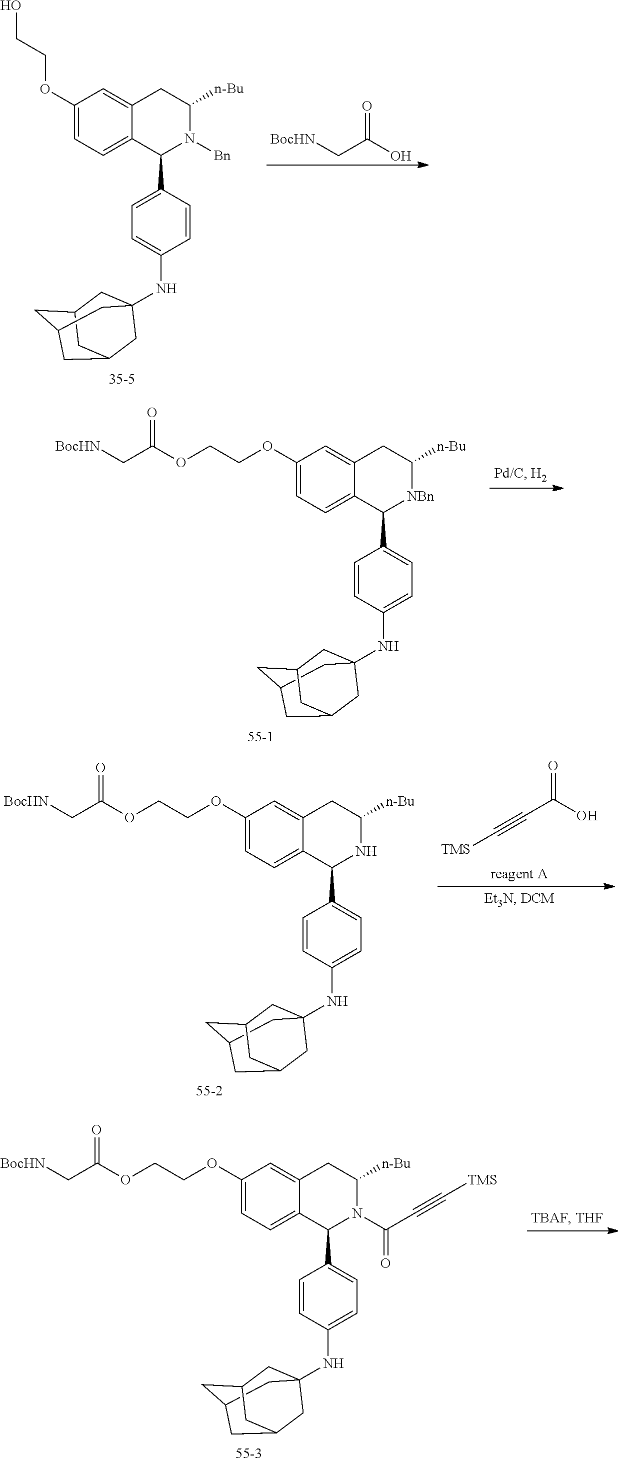

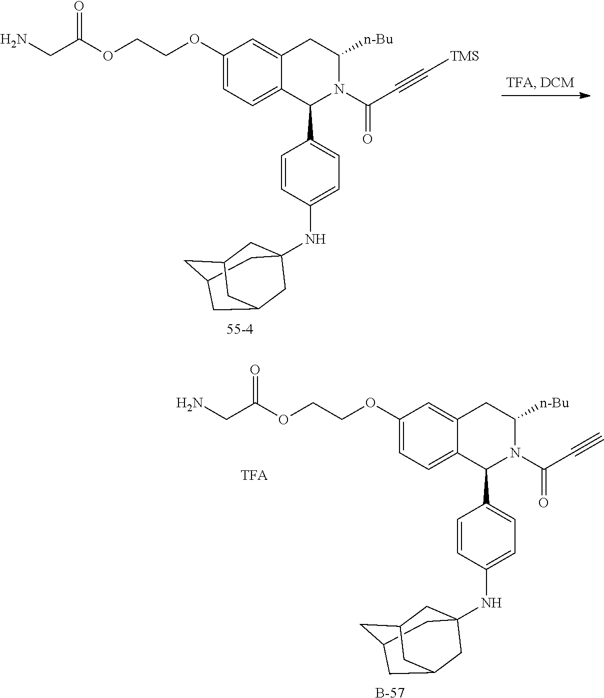

General Procedure for the Preparation of 55-1 To a solution of 2-(tert-butoxycarbonylamino)acetic acid (85 mg, 485.21 μmol, 1.5 eq) and 35-5 (182.70 mg, 323.47 μmol, 1 eq) in DCM (5 mL) was added TEA (49.10 mg, 485.21 μmol, 67.53 μL, 1.5 eq), HOBt (65.56 mg, 485.21 μmol, 1.5 eq) and EDCI (93.02 mg, 485.21 μmol, 1.5 eq), then the mixture was stirred at 40° C. for 16 h. LCMS and TLC (PE:EtOAc=3:1) showed the mixture was completed. The mixture was quenched by H₂O (10 mL), the organic layer was washed by NH₄Cl (10 mL). The organic layer was dried over anhydrous sodium sulfate and concentrated under reduced pressure to afford the crude product. The crude product was purified by silica column eluting with EtOAc/petroleum ether (5% to 15%) to afford 55-1 (0.23 g, 305.83 μmol, 94.55% yield).

### General Procedure for the Preparation of 55-2

55-1 (220 mg, 304.72 μmol, 1 eq) was dissolved in THF (2 mL) and MeOH (5 mL), Pd/C (304.72 ug, 304.72 μmol, 1 eq) was added into the reaction mixture. The system was flushed with H₂ and then evacuated and backfilled with H₂ (620.47 ug, 304.72 μmol, 1 eq) (3 times). The mixture was stirred at 20° C. for 16 h. LCMS and TLC (EtOAc:MeOH=10:1) showed the mixture was completed. The mixture was filtered and concentrated under reduced pressure to afford the crude product. The crude product was purified by silica column eluting with EtOAc/petroleum ether (50% to 80%) to afford 55-2 (90 mg, 142.44 μmol, 46.74% yield).

### General Procedure for the Preparation of 55-3

To a solution of 3-trimethylsilylprop-2-ynoic acid (5.97 g, 41.95 mmol, 1.08 eq) in DCM (100 mL) was added 2-chloro-1-methyl-pyridin-1-ium; iodide (10.92 g, 42.72 mmol, 1.1 eq), the mixture was stirred at 25° C. for 0.5 h. Then a solution of 55-2 (17.27 g, 38.84 mmol, 1 eq) and TEA (4.72 g, 46.61 mmol, 6.49 mL, 1.2 eq) in DCM (50 mL) was added dropwise at 0° C. The mixture was stirred at 0° C. for 1 h to give a yellow solution. LCMS and TLC (PE:EtOAc=3:1) showed the mixture was completed. The mixture was quenched by NH₄Cl (40 mL), the organic layer was washed by 1 M HCl (10 mL) and 1 M NHCO₃ (30 mL). The organic layer was dried over anhydrous sodium sulfate and concentrated under reduced pressure to afford the crude product. The crude product was purified by silica column eluting with EtOAc/petroleum ether (5% to 20%) to afford 55-3 (60 mg, 67.46 μmol, 47.36% yield).

### General Procedure for the Preparation of 55-4

55-3 (45 mg, 59.52 μmol, 1 eq) was dissolved in a mixture of THF (3 mL) under N₂. To the mixture was added TBAF (1 M, 89.28 μL, 1.5 eq) the mixture was stirred at −78° C. for 1 h. LCMS and TLC (PE:EtOAc=2:1) showed the mixture was completed. The mixture was quenched by NH₄Cl (10 mL), the organic layer was washed by NaCl (saturated, aq., 10 mL). The organic layer was dried over anhydrous sodium sulfate and concentrated under reduced pressure to afford the crude product. The crude product was purified by silica column eluting with EtOAc/petroleum ether (5% to 30%) and purified again by pre-TLC to afford 55-4 (28 mg, 39.71 μmol, 66.73% yield).

### General Procedure for Compound B-57

To a solution of 55-4 (28 mg, 40.94 μmol, 1 eq) in DCM (3 mL) was added TFA (46.68 mg, 409.43 μmol, 30.31 μL, 10 eq), then the mixture was stirred at 20° C. for 4 h. LCMS and TLC (PE:EtOAc=1:1) showed the mixture was completed. The mixture was concentrated by lyophilization to afford B-57 (22 mg, 29.32 μmol, 71.62% yield, TFA). LC-MS (m/z): 584.3 [M+H]⁺. ¹H NMR (400 MHz, CDCl₃) δ ppm 10.47 (br s, 1H), 8.23 (br s, 2H), 7.48-7.68 (m, 1H), 7.42 (br s, 1H), 7.21 (br s, 1H), 6.79-6.91 (m, 1H), 6.10-6.41 (m, 1H), 4.54-4.81 (m, 1H), 4.48 (br s, 1H), 4.19 (br s, 1H), 3.86 (br s, 1H), 2.65-2.96 (m, 1H), 2.10 (br s, 3H), 1.76 (br s, 4H), 1.46-1.66 (m, 5H), 1.11-1.32 (m, 6H), 0.83 (br t, J=6.65 Hz, 3H).

### Procedure 49: Compound B-58

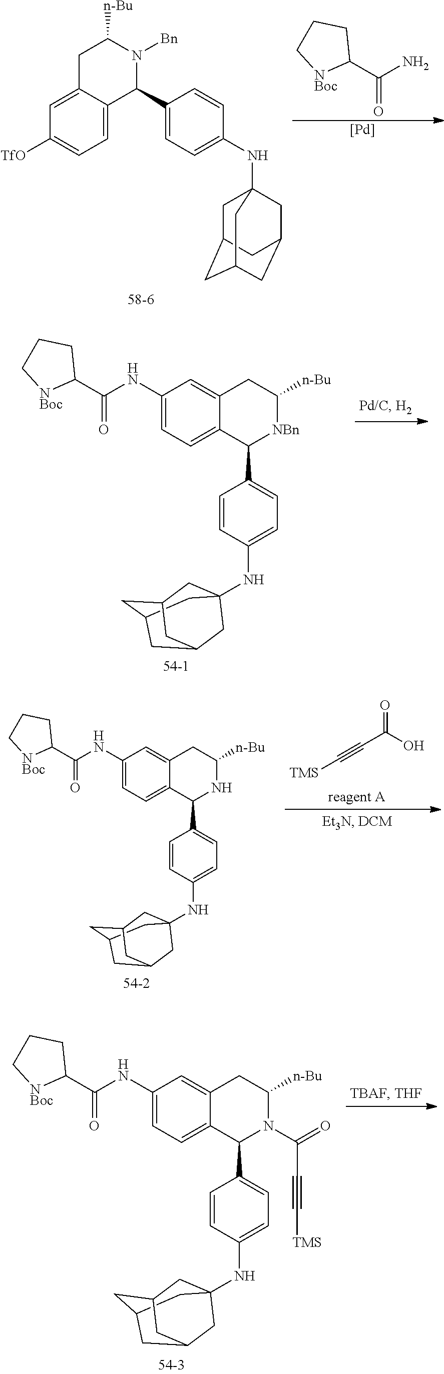

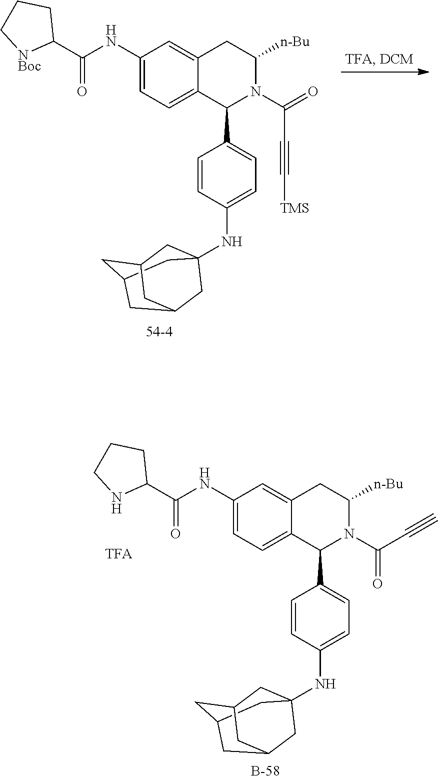

### General Procedure for the Preparation of 54-1

An oven-dried tube was charged with 58-6 (350 mg, 536.15 μmol, 1 eq), tert-butyl 2-carbamoylpyrrolidine-1-carboxylate (137.85 mg, 643.38 μmol, 1.2 eq), Cs₂CO₃ (349.37 mg, 1.07 mmol, 2 eq), Pd₂(dba)₃ (49.10 mg, 53.61 μmol, 0.1 eq), (5-diphenylphosphanyl-9,9-dimethyl-xanthen-4-yl)-diphenyl-phosphane (31.02 mg, 53.61 μmol, 0.1 eq) and dioxane (2 mL). The tube was evacuated and filled with nitrogen. Then the tube was sealed, the mixture was stirred at 100° C. for 16 h. LCMS and TLC (PE/EtOAc=3:1) showed the reaction was completed. The reaction mixture were filtered and concentrated under reduced pressure to afford the crude product. The crude product was purified by silica column eluting with EtOAc/petroleum ether (2% to 20%) to afford 54-1 (147 mg, 205.02 μmol, 38.24% yield).

### General Procedure for the Preparation of 54-2

54-1 (147 mg, 205.02 μmol, 1 eq) was dissolved in MeOH (2 mL) and THF (2 mL). Pd(OH)₂ (10 M, 2.05 μL, 0.1 eq) was added into the reaction mixture. The system was flushed with H₂ and then evacuated and backfilled with H₂ (3 times). The mixture was stirred at 20° C. for 16 h. LCMS and TLC (PE/EtOAc=3:1) showed the reaction was completed. The crude product was used to the next step without purification.

### General Procedure for the Preparation of 54-3

To a solution of 3-trimethylsilylprop-2-ynoic acid (28.59 mg, 201.00 μmol, 1.05 eq) in DCM (5 mL) was added 2-chloro-1-methyl-pyridin-1-ium; iodide (53.80 mg, 210.57 μmol, 1.1 eq), the mixture was stirred at 25° C. for 0.5 h. Then a solution of 54-2 (120 mg, 191.43 μmol, 1 eq) and TEA (23.24 mg, 229.71 μmol, 31.97 μL, 1.2 eq) in DCM (3 mL) was added dropwise at 0° C. The mixture was stirred at 0° C. for 1 h to give a yellow solution. LCMS and TLC (PE/EtOAc=3:1) showed the reaction was completed. The mixture was quenched by NH₄Cl (10 mL), the organic layer was washed by 1 M HCl (3 mL) and 1 M NHCO₃ (10 mL). The organic layer was dried over anhydrous sodium sulfate and concentrated under reduced pressure to afford the crude product. The crude product was purified by silica column eluting with EtOAc/petroleum ether (5% to 30% to 80%) to 54-3 (70 mg, 93.20 μmol, 48.69% yield).

### General Procedure for the Preparation of 54-4

54-3 (65 mg, 86.54 μmol, 1 eq) was dissolved in a mixture of THF (5 mL) under N₂. To the mixture was added TBAF (1 M, 86.54 μL, 1 eq) the mixture was stirred at −78° C. for 1 h. LCMS and TLC (PE/EtOAc=3:1) showed the reaction was completed. The mixture was quenched by NH₄Cl (saturated, aq., 1 mL), the organic layer was washed by NaCl (saturated, aq., 10 mL). The organic layer was dried over anhydrous sodium sulfate and concentrated under reduced pressure to afford the crude product. The crude product was purified by silica column eluting with EtOAc/petroleum ether (5% to 50%) to afford the crude product. The purified crude product was purified again by pre-TLC (PE:EA=1:1) to afford 30 mg of the crude product. The purified crude product was purified again by pre-TLC (PE:EA=1:1) to afford 54-4 (28 mg, 41.24 μmol, 47.66% yield).

### General Procedure for the Preparation of Compound B-58

To a solution of 54-4 (28 mg, 41.24 μmol, 1 eq) in DCM (3 mL) was added TFA (47.03 mg, 412.43 μmol, 30.54 μL, 10 eq) and after 1 hours was added TFA (1.54 g, 13.51 mmol, 1 mL, 327.47 eq), then the mixture was stirred at 20° C. for 2 h. LCMS showed the reaction was completed. The mixture was concentrated by lyophilization to afford B-58 (20 mg, 28.87 μmol, 69.99% yield, TFA). LC-MS (m/z): 579.2 [M+H]⁺. ¹H NMR (400 MHz, CDCl₃) δ ppm 10.57-10.62 (m, 1H), 9.40 (br s, 1H), 8.65 (br s, 1H), 7.52-7.62 (m, 2H), 7.38-7.52 (m, 3H), 7.22 (br s, 2H), 6.13-6.43 (m, 1H), 4.79 (br s, 1H), 4.70 (s, 1H), 4.32 (br s, 1H), 3.12-3.32 (m, 2H), 2.82-2.95 (m, 1H), 2.32-2.41 (m, 1H), 2.11 (br s, 3H), 1.94 (br s, 3H), 1.77 (br s, 6H), 1.62 (br d, J=12.05 Hz, 3H), 1.47-1.58 (m, 5H), 1.18-1.31 (m, 6H), 0.79-0.89 (m, 4H).

### Procedure 50: Compound B-59

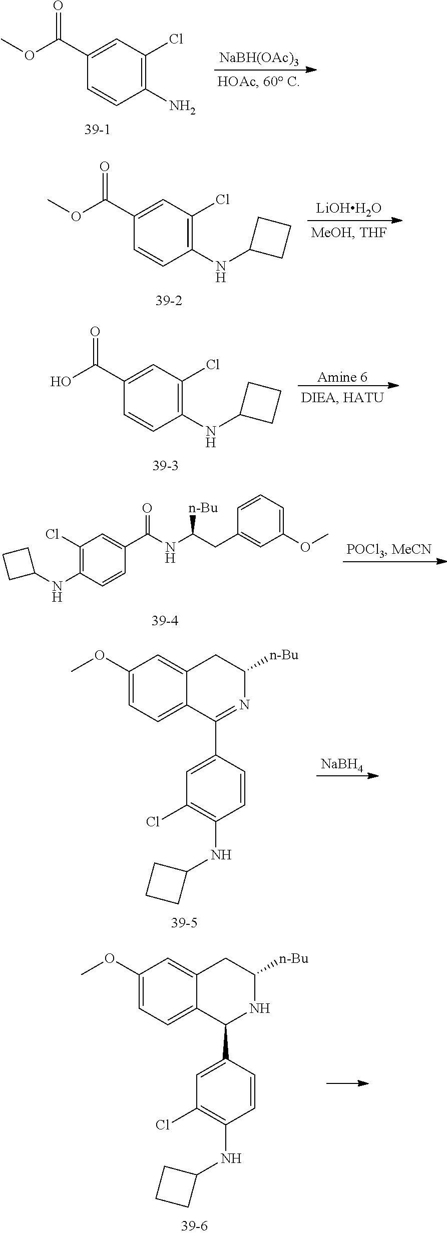

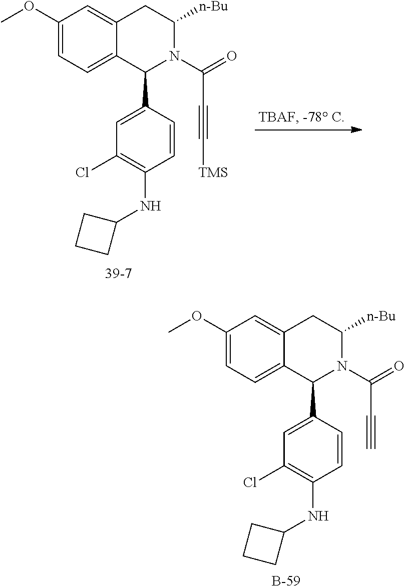

### General Procedure for the Preparation of Compound 39-2

To a solution of 39-1 (7 g, 37.71 mmol, 1 eq) in DCE (90 mL) was added cyclobutanone (3.17 g, 45.26 mmol, 3.38 mL, 1.2 eq), HOAc (2.26 g, 37.71 mmol, 2.16 mL, 1 eq). The reaction was stirred at 25° C. for 0.5 hours, then it was added NaBH(OAc)3 (7.99 g, 37.71 mmol, 1 eq) at 25° C. The reaction mixture was stirred at 25° C. for 12 hr to give solution. LCMS showed the reaction was completed. The reaction was basified with saturated NaHCO₃ aq. (50 mL), extracted with DCM (30 mL×3). The combined organic layers were dried over Na₂SO₄, filtered and concentrated under reduced pressure. The crude product was purified by flash column (SiO2, PE to EA 5% in PE). 39-3 (3.81 g, 15.90 mmol, 42.15% yield).

### General Procedure for the Preparation of Compound 39-3

To a solution of 39-3 (2.81 g, 11.72 mmol, 1 eq) in MeOH (5 mL) and H2O (2.5 mL) was added LiOH·H2O (4.92 g, 117.25 mmol, 10 eq) at 25° C. The mixture was stirred at 45° C. for 16 h to give a yellow solution. LCMS showed the reaction was completed. The reaction was diluted with H₂O (30 mL) and extracted with MBTE (30 mL). The water layer was acidified to pH=5˜6 and extracted with EtOAc (30 mL*3). The organic layers were dried over Na₂SO₄ and concentrated in vacuum and used for next step without purification. 39-4 (2.46 g, 9.80 mmol, 83.57% yield).

### General Procedure for the Preparation of Compound 39-4

To a solution of 39-3 in DCM (5 mL) was added HATU (935.87 mg, 2.46 mmol, 1.2 eq) and DIEA (795.25 mg, 6.15 mmol, 1.07 mL, 3 eq). The mixture was stirred at 15° C. for 1 h. Then (2S)-1-(3-methoxyphenyl) hexan-2-amine (500 mg, 2.05 mmol, 1 eq, HCl) in DCM (5 mL) was added. The reaction was stirred at 15° C. for 12 hr to give a yellow solution. LCMS showed the reaction was completed. The reaction mixture was quenched with H₂O (30 mL) and extracted with DCM (30 mL*3). The organic layers were dried over Na₂SO₄ and concentrated to give the crude product. The residue was purified by column chromatography (SiO₂, petroleum ether/ethyl acetate=100/0 to 69/31) to give 39-4 (1.02 g, crude).

### General Procedure for the Preparation of Compound 39-5

To a solution of 39-4 (700 mg, 1.69 mmol, 1 eq) in DCM (15 mL) was added 2-chloropyridine (766.18 mg, 6.75 mmol, 638.48 μL, 4 eq) and Tf₂O (1.90 g, 6.75 mmol, 1.11 mL, 4 eq) at −78° C. under N₂. After 15 min, the reaction mixture was placed in an ice-water bath and warmed to 0° C. After 15 min, the resulting solution was allowed to warm to 20° C. The reaction was allowed to stir at 20° C. under N₂ for 12 hr to give a yellow solution. LCMS showed the reaction was completed. The mixture was quenched with saturated NaHCO₃ (15 mL), extracted with DCM (15 mL*3). The organic layers were dried over sodium sulfate, filtered and concentrated under vacuum. The residue was purified by column chromatography (SiO₂, petroleum ether/ethyl acetate=100/0 to 60/40) to give 39-5 (760 mg).

### General Procedure for the Preparation of Compound 39-6

To a solution of 39-5 (760 mg, 1.91 mmol, 1 eq) in MeOH (15 mL) was added NaBH₄ (362.14 mg, 9.57 mmol, 5 eq) at 50° C. The reaction was stirred 1 hr to give a yellow solution. LCMS showed the reaction was completed. The mixture was added NaHCO₃ (20 mL) and filtered. Then extracted with EtOAc (20 mL*3). The organic layers were dried over Na₂SO₄, filtered and concentrated under vacuum. The crude product was purified by column chromatography (SiO₂, petroleum ether/ethyl acetate=100/0 to 90/10) to give 39-6 (70 mg, 175.45 μmol, 9.16% yield).

### General Procedure for the Preparation of Compound 39-7

To a solution of 3-trimethylsilylprop-2-ynoic acid (24.95 mg, 175.45 μmol, 1 eq) in DCM (2 mL) was added 2-chloro-1-methyl-pyridin-1-ium; iodide (44.83 mg, 175.45 μmol, 1 eq). The reaction was stirred at 20° C. for 0.5 hr. Then the mixture was added dropwise in solution of 39-6 (70 mg, 175.45 μmol, 1 eq) and TEA (35.51 mg, 350.91 μmol, 48.84 μL, 2 eq) in DCM (2 mL) at 0° C. The reaction was stirred at 0° C. for 1 hr to give a yellow solution. LCMS showed the reaction was completed. The reaction mixture was washed with saturated NH₄Cl (10 mL) and extracted with DCM (10 mL*3). The organic layers were dried over Na₂SO₄ and concentrated to give the crude product. The residue was purified by column chromatography (SiO2, Petroleum ether/Ethyl acetate=100/0 to 75/25) to give 39-7 (50 mg, 95.57 μmol, 54.47% yield).

### General Procedure for the Preparation of B-59

To a solution of 39-7 (50.00 mg, 95.57 μmol, 1 eq) in THF (3 mL) was added TBAF (1 M, 105.13 μL, 1.1 eq) at −78° C. The reaction was stirred at −78° C. for 15 min to give a yellow solution. TLC (PE/EtOAc=3:1) showed the reaction was completed. The reaction mixture was quenched H₂O (10 mL) and extracted with EtOAc (10 mL*3). The organic layers were dried over Na₂SO₄ and concentrated to give the crude product. The residue was purified by column chromatography (SiO₂, Petroleum ether/Ethyl acetate=100/0 to 74/26) to give B-59 (22.84 mg, 50.64 μmol, 52.99% yield). LC-MS (m/z): 451.2[M+H]⁺. ¹H NMR (CDCl₃, 400 MHz): δ=7.29 (s, 1H), 7.18 (d, J=8.3 Hz, 1H), 7.06-6.98 (m, 1H), 6.98-6.87 (m, 1H), 6.80 (ddd, J=17.0, 8.2, 2.4 Hz, 1H), 6.69 (d, J=2.0 Hz, 1H), 6.60-6.45 (m, 1H), 6.25-6.09 (m, 1H), 4.67-4.44 (m, 1H), 3.91-3.78 (m, 4H), 3.17-3.06 (m, 1H), 2.99-2.79 (m, 1H), 2.78-2.65 (m, 1H), 2.45-2.30 (m, 2H), 1.97-1.77 (m, 4H), 1.34-1.05 (m, 6H), 0.84 ppm (q, J=6.8 Hz, 3H).

### Procedure 51: Compound B-61

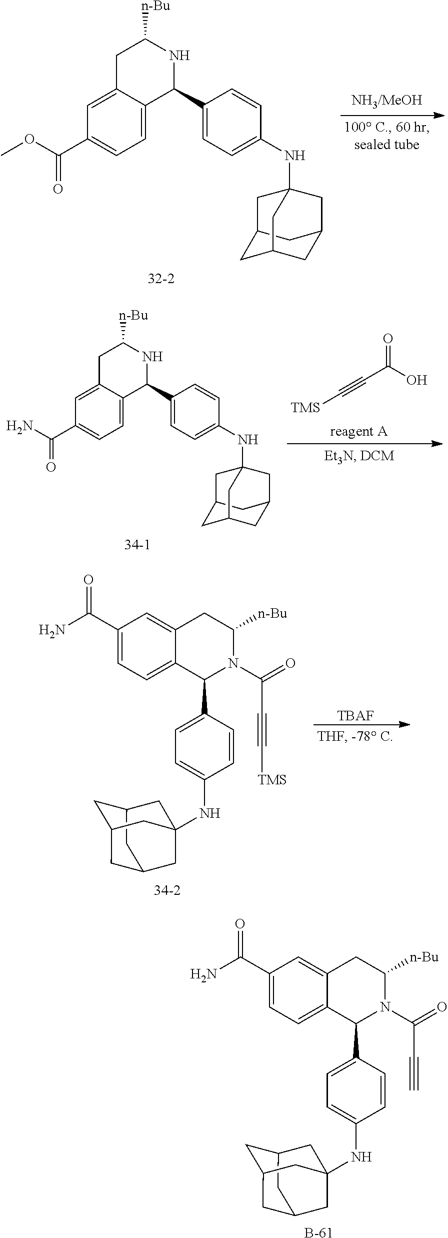

### General Procedure for the Preparation of Compound 34-1

A solution of 32-2 (320 mg, 677.02 μmol, 1 eq) in NH₃/MEOH (7 M, 40 mL, 413.58 eq) was stirred at 100° C. for 36 hr in a 100 mL of sealed tube to give a yellow solution. LCMS and TLC (PE:EtOAc=0:1) showed most 32-2 remained and desired mass was found. The mixture was concentrated under vacuum. The residue was purified by column chromatography (SiO₂, Petroleum ether/Ethyl acetate=100/0 to 71/29 and Ethyl acetate/MeOH (NH₃·H₂O)=100/0 to 90/10) to give 32-2 (114 mg, 241.19 μmol, 35.63% yield) and 34-1 (130 mg, 284.06 μmol, 41.96% yield).

### General Procedure for the Preparation of Compound 34-2

To a solution of 3-trimethylsilylprop-2-ynoic acid (19.89 mg, 139.85 μmol, 0.8 eq) in DCM (2 mL) was added 2-chloro-1-methyl-pyridin-1-ium; iodide (44.66 mg, 174.81 μmol, 1 eq). The mixture was stirred at 20° C. for 1 hr to give a yellow suspension. The result mixture was dropwise to a solution of 34-1 (80 mg, 174.81 μmol, 1 eq) and TEA (17.69 mg, 174.81 μmol, 24.33 μL, 1 eq) in DCM (2 mL) at 0° C. It was stirred at 0° C. for 1 hr to give a yellow solution. LCMS and TLC (EtOAc:MeOH=3:1) showed 34-1 and desired product was found. The mixture was quenched with aq·NH₄Cl (8 mL), extracted with DCM (10 mL*3). The organic layer was separated and dried over sodium sulfate, filtered and concentrated under vacuum. The residue was purified by prep-TLC (SiO₂, petroleum ether/ethyl acetate=1:1.5) to give 34-2 (40 mg, 63.50 μmol, 36.33% yield).

### General Procedure for the Preparation of Compound B-61

To a solution of 34-2 (40 mg, 68.74 μmol, 1 eq) in THF (3 mL) were added TBAF (1 M, 75.62 μL, 1.1 eq) at −78° C. The mixture was stirred at −78° C. for 20 mins to give a clear solution. TLC (PE:EtOAc=1:2) showed the mixture was completed. The mixture was added H₂O (8 mL), extracted with EtOAc (8 mL*3). The organic layers were dried over sodium sulfate, filtered and concentrated under vacuum. The residue was purified by prep-TLC (SiO₂, Petroleum ether/Ethyl acetate=1:1) to give B-61 (28.21 mg, 54.14 μmol, 78.75% yield). LC-MS (m/z): 510.1 [M+H]⁺. ¹H NMR (400 MHz, CDCl₃) δ ppm 7.84-7.61 (m, 2H) 7.59-7.37 (m, 1H) 7.25-6.80 (m, 4H) 6.50-6.00 (m, 2H) 5.71 (br s, 1H) 4.80-4.45 (m, 1H) 3.28-2.72 (m, 3H) 2.14-1.95 (m, 4H) 1.93-1.78 (m, 5H) 1.61 (br s, 6H) 1.34-1.06 (m, 6H) 0.91-0.80 (m, 3H).

### Procedure 52: Compound B-62

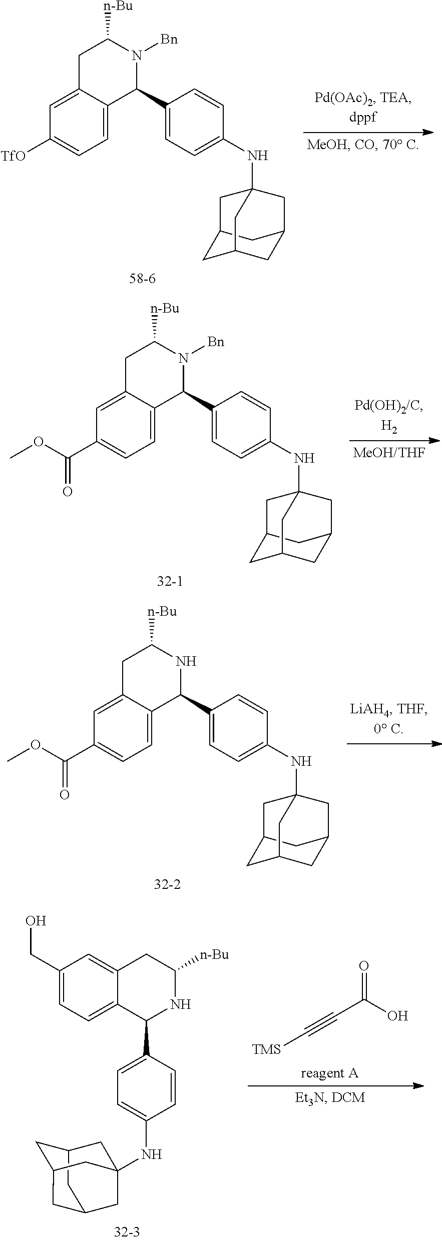

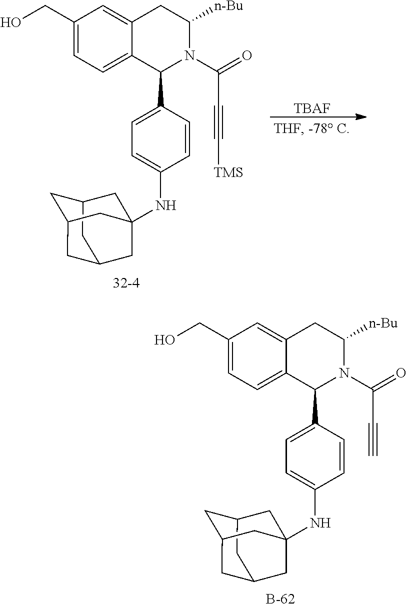

### General Procedure for the Preparation of Compound 32-1

To a suspension of 58-6 (4.1 g, 6.28 mmol, 1 eq) in MeOH (70 mL) was added Pd(OAc)₂ (705.02 mg, 3.14 mmol, 0.5 eq), TEA (3.81 g, 37.68 mmol, 5.25 mL, 6 eq) and DPPF (1.74 g, 3.14 mmol, 0.5 eq). The suspension was degassed under vacuum and purged with CO (6.28 mmol, 1 eq) several times. The mixture was stirred under CO (50 psi) at 80° C. for 12 hours to give a yellow suspension. TLC (PE:EtOAc=4:1) showed the mixture was completed. The mixture was filtered through celite, and washed with MeOH (100 mL). The filtrate was concentrated under vacuum. The residue was diluted with EtOAc (40 mL). The mixture was added H₂O (40 mL), extracted with EtOAc (40 mL*3). The organic layers were dried over sodium sulfate, filtered and concentrated under vacuum. The residue was purified by column chromatography (SiO₂, Petroleum ether/Ethyl acetate=100/0 to 92/8) to give 32-1 (2.63 g, 4.67 mmol, 74.41% yield).

### General Procedure for the Preparation of Compound 32-2

To a solution of 32-1 (584 mg, 1.04 mmol, 1 eq) in MeOH (20 mL)/THF (5 mL) was added Pd(OH)₂/C (130.00 mg, 92.57 μmol, 8.92 e-2 eq) under N₂. The suspension was degassed under vacuum and purged with H₂ (164.76 ug, 81.74 μmol) several times. The mixture was stirred under H₂ (15 psi) at 10° C. for 12 hours to give a black suspension. LCMS and TLC (PE:EtOAc=3:1) showed the mixture was completed. The mixture was filtered through celite. The filtrated was added TEA (2 mL) and concentrated under vacuum. The residue was purified by column chromatography (SiO₂, Petroleum ether/Ethyl acetate=100/0 to 75/25) to give 32-2 (410 mg, 867.43 μmol, 83.59% yield).

### General Procedure for the Preparation of Compound 32-3

To a solution of 32-2 (255 mg, 539.50 μmol, 1 eq) in THF (5 mL) was dropwise LiAlH₄ (81.91 mg, 2.16 mmol, 4 eq) at 0° C. under N₂. The mixture was stirred at 0° C. for 2 hr to give a yellow suspension. LCMS showed the mixture was completed. The reaction was cooled to 0° C. and quenched with H₂O (80 μL), 15% NaOH (80 μL) and H₂O (240 μL), dried over sodium sulfate, filtered. The filtrate was concentrated. Used next step without further purification. 32-3 (200 mg, 449.79 μmol, 83.37% yield).

### General Procedure for the Preparation of Compound 32-4

To a solution of 3-trimethylsilylprop-2-ynoic acid (63.97 mg, 449.79 μmol, 1 eq) in DCM (2 mL) was added 2-chloro-1-methyl-pyridin-1-ium; iodide (114.91 mg, 449.79 μmol, 1 eq). The mixture was stirred at 10° C. for 0.5 hr to give a yellow suspension. The result mixture was dropwise to a solution of 32-3 (200 mg, 449.79 μmol, 1 eq) and TEA (45.51 mg, 449.79 μmol, 62.61 μL, 1 eq) in DCM (4 mL) at 0° C. It was stirred at 0° C. for 1 hr to give a yellow solution. LCMS and TLC (PE:EtOAc=1:1) showed the mixture was completed. The mixture was quenched with aq·NH₄Cl (10 mL), extracted with DCM (10 mL*3). The organic layer was separated and dried over sodium sulfate, filtered and concentrated under vacuum. The residue was purified by column chromatography (SiO₂, Petroleum ether/Ethyl acetate=100/0 to 65/35) to give 32-4 (142 mg, 227.94 μmol, 50.68% yield).

### General Procedure for the Preparation of Compound B-62

To a solution of 32-4 (15 mg, 26.37 μmol, 1 eq) in THF (3 mL) were added TBAF (1 M, 29.01 μL, 1.1 eq) at −78° C. The mixture was stirred at −78° C. for 20 mins to give a clear solution. LCMS and TLC (PE:EtOAc=1:2) showed the mixture was completed. The mixture was added H₂O (8 mL), extracted with EtOAc (8 mL*3). The organic layers were dried over sodium sulfate, filtered and concentrated under vacuum. The residue was purified by prep-TLC (SiO₂, Petroleum ether/Ethyl acetate=1:1) to give B-62 (3.52 mg, 7.09 μmol, 26.88% yield). LC-MS (m/z): 497.2 [M+H]⁺. ¹H NMR (400 MHz, CDCl₃) δ ppm 7.41-7.28 (m, 1H) 7.26-7.21 (m, 1H) 7.16 (s, 1H) 6.99-6.81 (m, 2H) 6.71-6.52 (m, 2H) 6.36-6.15 (m, 1H) 4.77-4.38 (m, 3H) 3.25-2.86 (m, 2H) 2.86-2.70 (m, 1H) 2.13-1.99 (m, 3H) 1.88-1.75 (m, 6H) 1.68-1.60 (m, 6H) 1.35-1.11 (m, 6H) 0.94-0.80 (m, 3H).

### Procedure 53: Compound B-63

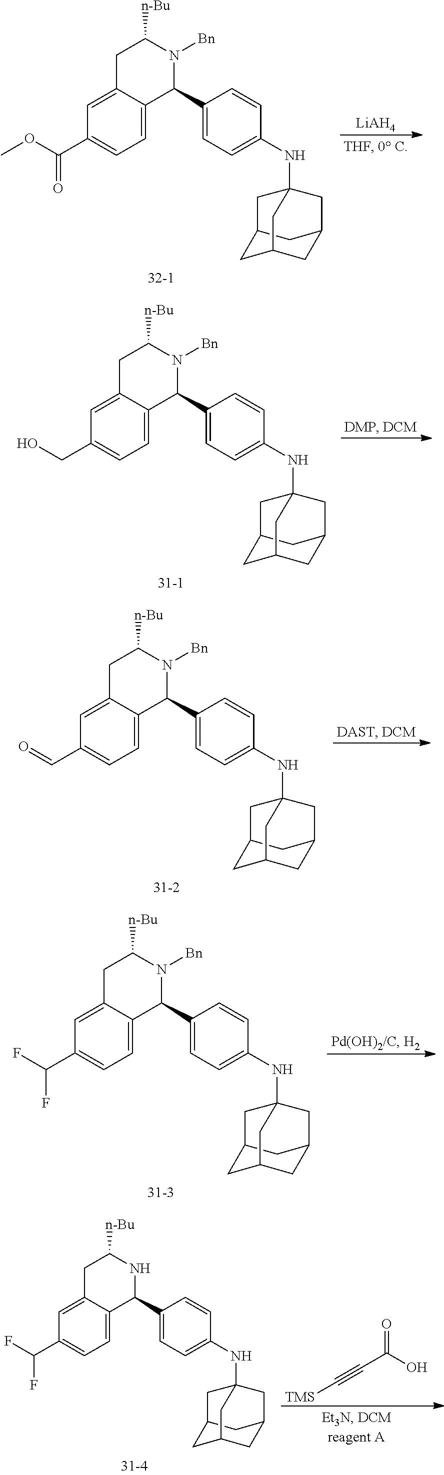

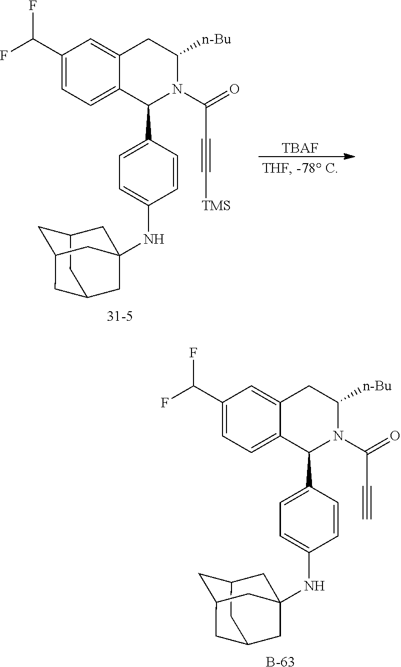

### General Procedure for the Preparation of Compound 31-1

To a solution of 32-1 (200 mg, 355.38 μmol, 1 eq) in THF (5 mL) was dropwise LiAlH₄ (40.46 mg, 1.07 mmol, 3 eq) at 0° C. under N₂. The mixture was stirred at 0° C. for 2 hr to give a white suspension. LCMS showed the mixture was completed. The reaction was cooled to 0° C. and quenched with H₂O (40 μL), 15% NaOH (40 μL) and H₂O (120 μL), dried over sodium sulfate, filtered. The filtrate was concentrated. Used next step without further purification. 31-1 (182 mg, 340.33 μmol, 95.77% yield).

### General Procedure for the Preparation of Compound 31-2

To a solution of 31-1 (182 mg, 340.33 μmol, 1 eq) in DCM (5 mL) was added Dess-Martin (173.22 mg, 408.40 μmol, 126.44 μL, 1.2 eq). The mixture was stirred at 20° C. for 12 hr to give a red solution. LCMS and TLC (PE:EtOAc=1:1) showed a little 31-1 remained and desired mass was found. The mixture was quenched with saturated Na₂SO₃ (20 mL), extracted with DCM (25 mL*3). The organic layers were dried over sodium sulfate, filtered and concentrated under vacuum. The residue was purified by column chromatography (SiO₂, Petroleum ether/Ethyl acetate=100/0 to 90/10) to give 31-2 (120 mg, 225.24 μmol, 66.18% yield).

### General Procedure for the Preparation of Compound 31-3

To a solution of 31-2 (100 mg, 187.70 μmol, 1 eq) in DCM (5 mL) was added DAST (302.56 mg, 1.88 mmol, 248.00 μL, 10 eq). The mixture was stirred at 20° C. for 12 hr to give a red solution. LCMS showed most 31-2 remained. Then it was added DAST (302.56 mg, 1.88 mmol, 248.00 μL, 10 eq). The mixture was stirred at 40° C. for 2 hr. LCMS and TLC (PE:EtOAc=3:1) showed a little 31-2 remained and desired mass was found. The crude products were combined for work up. The mixture was quenched with saturated NaHCO₃ (10 mL), extracted with DCM (10 mL*3). The organic layers were dried over sodium sulfate, filtered and concentrated under vacuum. The residue was purified by column chromatography (SiO₂, Petroleum ether/Ethyl acetate=100/0 to 90/10) to give 31-3 (120 mg).

### General Procedure for the Preparation of Compound 31-4

To a solution of 31-3 (50 mg, 90.13 μmol, 1 eq) in THF (10 mL) was added Pd(OH)₂ (30 mg, 21.36 μmol, 2.37 e-1 eq) under N₂. The suspension was degassed under vacuum and purged with H₂ (72.67 μg, 36.05 μmol) several times. The mixture was stirred under H₂ (15 psi) at 20° C. for 12 hours to give a black suspension. LCMS showed 31-3 remained and desired mass was found. The mixture was filtered through Celite. The residue was purified by column chromatography (SiO₂, Petroleum ether/Ethyl acetate=100/0 to 55/45) to give 31-3 (20 mg, 36.05 μmol, 40.00% yield) and 31-4 (14 mg, 30.13 μmol, 33.43% yield).

### General Procedure for the Preparation of Compound 31-5

To a solution of 3-trimethylsilylprop-2-ynoic acid (4.29 mg, 30.13 μmol, 1 eq) in DCM (1 mL) was added 2-chloro-1-methyl-pyridin-1-ium; iodide (7.70 mg, 30.13 μmol, 1 eq). The mixture was stirred at 20° C. for 1 hr to give a yellow suspension. The result mixture was dropwise to a solution of 31-4 (14 mg, 30.13 μmol, 1 eq) and TEA (3.05 mg, 30.13 μmol, 4.19 μL, 1 eq) in DCM (2 mL) at 0° C. It was stirred at 0° C. for 1 hr to give a yellow solution. LCMS and TLC (PE:EtOAc=0:1) showed the mixture was completed. The mixture was quenched with aq·NH₄Cl (8 mL), extracted with DCM (8 mL*3). The organic layer was separated and dried over sodium sulfate, filtered and concentrated under vacuum. Used next step without further purification. 31-5 (25 mg, crude).

### General Procedure for the Preparation of Compound B-63

To a solution of 31-5 (25 mg, 42.46 μmol, 1 eq) in THF (3 mL) were added TBAF (1 M, 46.70 μL, 1.1 eq) at −78° C. The mixture was stirred at −78° C. for 10 mins to give a clear solution. LCMS and TLC (PE:EtOAc=3:2) showed the mixture was completed. The mixture was added H₂O (6 mL), extracted with EtOAc (6 mL*3). The organic layers were dried over sodium sulfate, filtered and concentrated under vacuum. The residue was purified by prep-TLC (SiO₂, Petroleum ether/Ethyl acetate=3:2) to give B-63 (7.07 mg, 12.39 μmol, 29.19% yield). LC-MS (m/z): 517.1 [M+H]⁺. ¹H NMR (400 MHz, CDCl₃) δ ppm 7.54-7.34 (m, 2H) 7.31 (s, 2H) 7.21-6.71 (m, 3H) 6.69-6.45 (m, 1H) 6.41-6.25 (m, 1H) 4.81-4.33 (m, 1H) 3.26-2.71 (m, 3H) 2.11-1.89 (m, 3H) 1.83 (br s, 6H) 1.70-1.57 (m, 6H) 1.36-1.08 (m, 6H) 0.90-0.79 (m, 3H).

### Procedure 54: Compound B-64

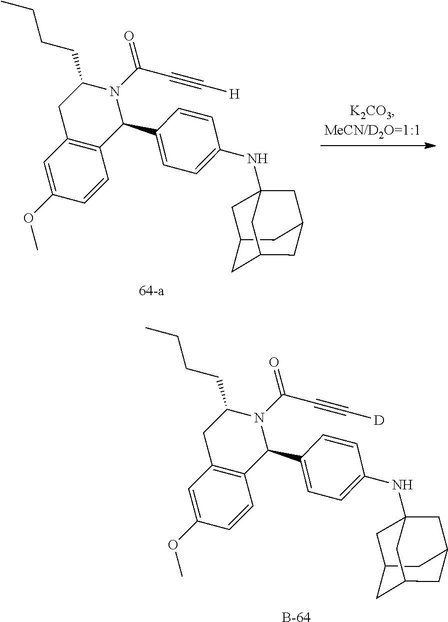

### General Procedure for the Preparation of Compound B-64

To a solution of 64-a (30 mg, 60.40 μmol, 1 eq) in MeCN (2 mL) was added K₂CO₃ (20.87 mg, 151.00 μmol, 2.5 eq). The reaction was stirred at 10° C. for 0.5 hr to give yellow solution. Then, the reaction was added D₂O (2 mL). The reaction was stirred at 10° C. for 0.5 hr to give a colorless solution. LCMS showed the reaction was completed. The reaction was extracted with DCM (5 mL×3). The organic layers were dried over Na₂SO₄ and concentrated to give the product. The product was concentrated to remove most of CH3CN, D₂O and lyophilized to give B-64 (16.75 mg, 32.75 μmol, 54.22% yield). LC-MS (m/z): 498.1 [M+H]⁺. ¹H NMR (400 MHz, acetonitrile-d₃) δ ppm 7.46-7.21 (m, 1H) 6.99-6.60 (m, 6H) 6.29-5.92 (m, 1H) 4.77-4.43 (m, 1H) 3.76 (d, J=5.63 Hz, 3H) 3.58-2.68 (m, 3H) 2.04 (br s, 3H) 1.83 (dd, J=9.01, 2.75 Hz, 6H) 1.72-1.59 (m, 6H) 1.46-1.04 (m, 6H) 0.92-0.79 (m, 3H).

### Procedure 55: Compound B-65

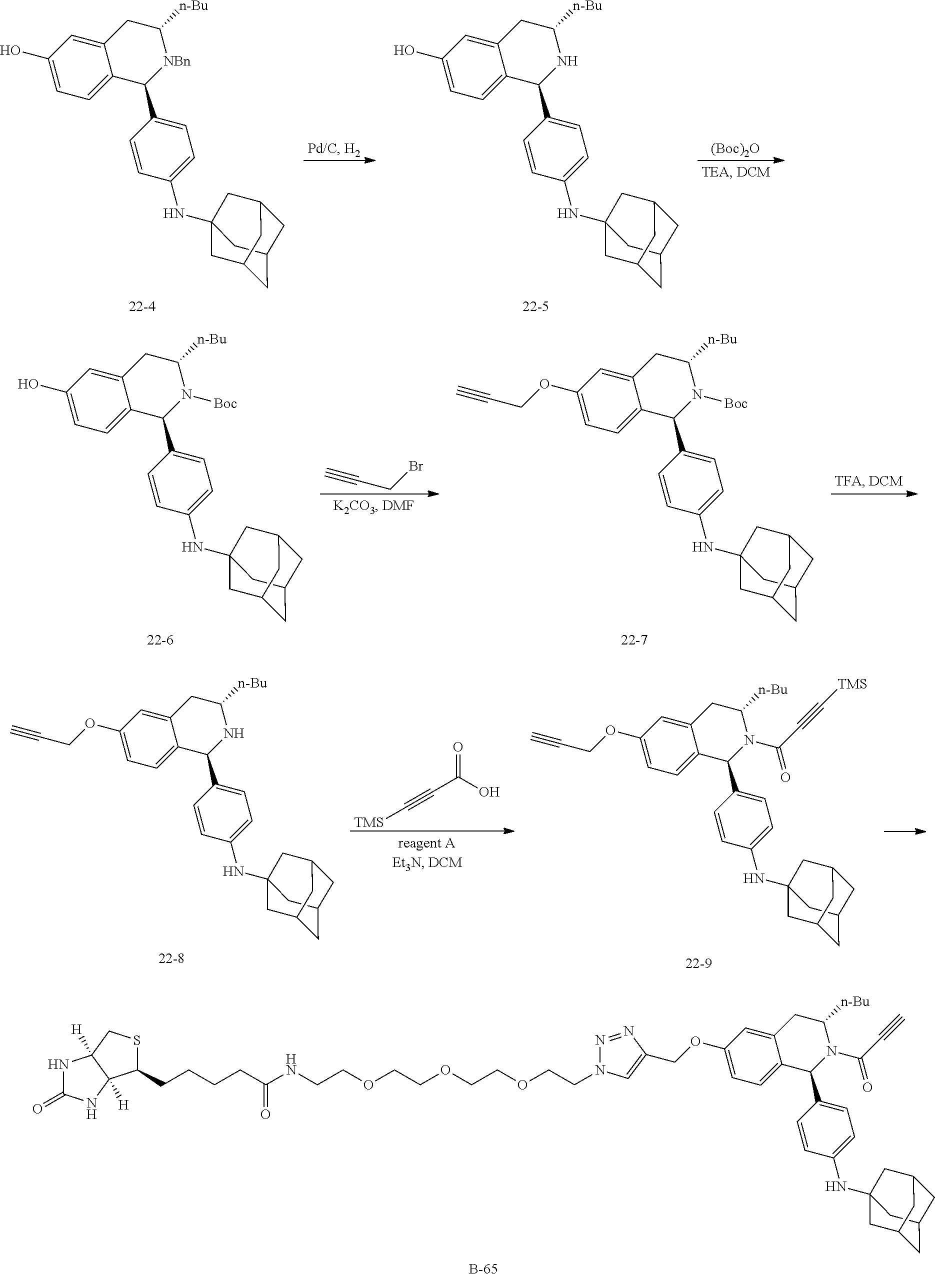

### General Procedure for the Preparation of Compound 22-5

To a solution of 22-4 in MeOH (15 mL) was added H₂ (1.75 mg, 864.15 μmol, 1 eq) under N₂. The suspension was degassed under vacuum and purged with H₂ several times. The mixture was stirred under Pd(OH)₂ (97.91 mg, 139.43 μmol, 1.61 e-1 eq) at 20° C. for 12 hr to give a black suspension. LCMS showed the reaction was completed. The reaction mixture was filtered on celite and washed with MeOH (50 mL). The filtrate was concentrated to give the crude product. The residue was purified by column chromatography (SiO₂, Petroleum ether/Ethyl acetate=1/0 to 64/36) to give 22-5 (210 mg, 487.67 μmol, 56.43% yield).

### General Procedure for the Preparation of Compound 22-6

To a solution of 22-5 (210 mg, 487.67 μmol, 1 eq) in DCM (5 mL) was added TEA (98.69 mg, 975.33 μmol, 135.75 μL, 2 eq) and tert-butoxycarbonyl tert-butyl carbonate (106.43 mg, 487.67 μmol, 112.03 μL, 1.00 eq) at 0° C. The mixture was warmed to 20° C. and stirred for 12 hr to give a yellow solution. LCMS showed the reaction was completed. The reaction was quenched with H₂O (10 mL). The separated aqueous layer was extracted with DCM (10 mL*2). The combined organic layers were washed with brine (10 mL) and dried over sodium sulfate, filtered and concentrated to give the crude product. The residue was purified by column chromatography (SiO₂, Petroleum ether/Ethyl acetate=100/0 to 74/26) to give 22-6 (190 mg, 357.99 μmol, 73.41% yield).

### General Procedure for the Preparation of Compound 22-7

To a solution of 22-6 (130 mg, 244.94 μmol, 1 eq) in DMF (4 mL) was added K₂CO₃ (50.78 mg, 367.41 μmol, 1.5 eq) and 3-bromoprop-1-yne (40.07 mg, 269.44 μmol, 29.03 μL, 1.1 eq). The mixture was stirred at 15° C. for 12 hr to give a yellow solution. LCMS showed the mixture was completed. The reaction mixture was quenched H₂O (10 mL) and extracted with EtOAc (10 mL*3). The organic layers were dried over Na₂SO₄ and concentrated to give 22-7 (200 mg, crude).

### General Procedure for the Preparation of Compound 22-8

To a solution of 22-7 (200 mg, 351.63 μmol, 1 eq) in DCM (5 mL) was added TFA (400.92 mg, 3.52 mmol, 260.34 μL, 10 eq). The reaction was stirred at 15° C. for 12 hr to give a yellow solution. LCMS showed the mixture was completed. The reaction mixture was washed with saturated NaHCO₃ (10 mL) and extracted with DCM (10 mL*3). The organic layers were dried over Na₂SO₄ and concentrated to give 22-8 (200 mg, crude) which was used for next step without purification.

### General Procedure for the Preparation of 22-9

To a solution of 3-trimethylsilylprop-2-ynoic acid (50.01 mg, 351.63 μmol, 1 eq) in DCM (3 mL) was added 2-chloro-1-methyl-pyridin-1-ium; iodide (89.84 mg, 351.63 μmol, 1 eq). The reaction was stirred at 20° C. for 0.5 hr. Then the mixture was added dropwise in solution of 22-8 (164.8 mg, 351.63 μmol, 1 eq) and TEA (71.16 mg, 703.26 μmol, 97.89 μL, 2 eq) in DCM (3 mL) at 0° C. The reaction was stirred at 0° C. for 1 hr to give a yellow solution. LCMS showed the reaction was completed. The reaction mixture was washed with saturated NH₄Cl (10 mL) and extracted with DCM (10 mL*3). The organic layers were dried over Na₂SO₄ and concentrated to give the crude product. The residue was purified by column chromatography (SiO2, Petroleum ether/Ethyl acetate=100/0 to 83/17) to give 22-9 (90 mg, 151.80 μmol, 43.17% yield).

### General Procedure for the Preparation of B-65

To a solution of 5-[(3aS,4S,6aR)-2-oxo-1,3,3a,4,6,6a-hexahydrothieno[3,4-d]imidazol-4-yl]-N-[2-[2-[2-(2-azidoethoxy)ethoxy]ethoxy]ethyl]pentanamide (67.48 mg, 151.80 μmol, 1 eq) in THF (3 mL)/H₂O (0.5 mL) was added 22-9 (90 mg, 151.80 μmol, 1 eq), CuSO4 (2.42 mg, 15.18 μmol, 2.33 μL, 0.1 eq) and sodium ascorbate (15.04 mg, 75.90 μmol, 0.5 eq) under N₂. The mixture was stirred at 40° C. for 12 hr to give a yellow solution. LCMS showed the reaction was completed. The mixture was concentrated to give the crude product. The residue was purified by prep-HPLC (HCl condition; column: 3_Phenomenex Luna C18 75*30 mm*3 μm; mobile phase: [water (10 mM HCl)-ACN]; B %: 17%-47%, 9.5 min) to give B-65 (15.54 mg, 15.33 μmol, 10.10% yield). LC-MS (m/z): 965.5[M+H]⁺. ¹H NMR (CDCl₃, 400 MHz): δ=11.04 (br s, 1H), 8.46 (br s, 1H), 7.62 (br d, J=16.1 Hz, 1H), 7.53-7.30 (m, 4H), 7.20 (br s, 1H), 6.92 (br d, J=7.6 Hz, 1H), 6.82 (br s, 1H), 6.46 (br s, 1H), 4.71 (br s, 2H), 4.61-4.26 (m, 3H), 3.98-3.79 (m, 2H), 3.66-3.48 (m, 8H), 3.44-3.32 (m, 2H), 3.26-3.04 (m, 2H), 2.92-2.70 (m, 2H), 2.66 (br d, J=1.2 Hz, 12H), 2.29-1.85 (m, 12H), 1.69-1.52 (m, 8H), 1.46-1.03 (m, 6H), 0.93-0.69 ppm (m, 3H).

### Procedure 56: Compounds B-67 and B-68

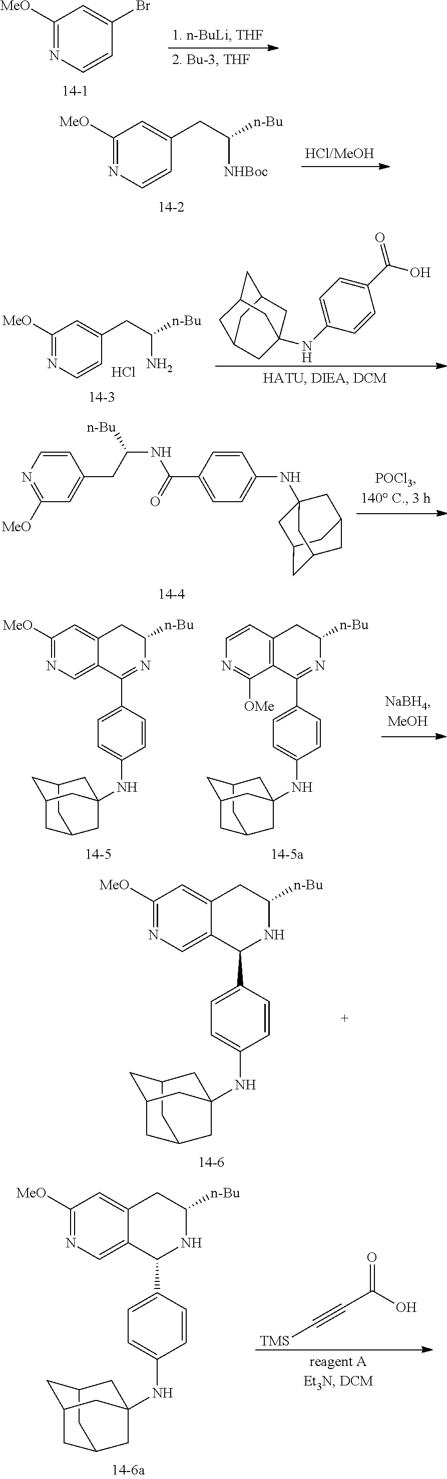

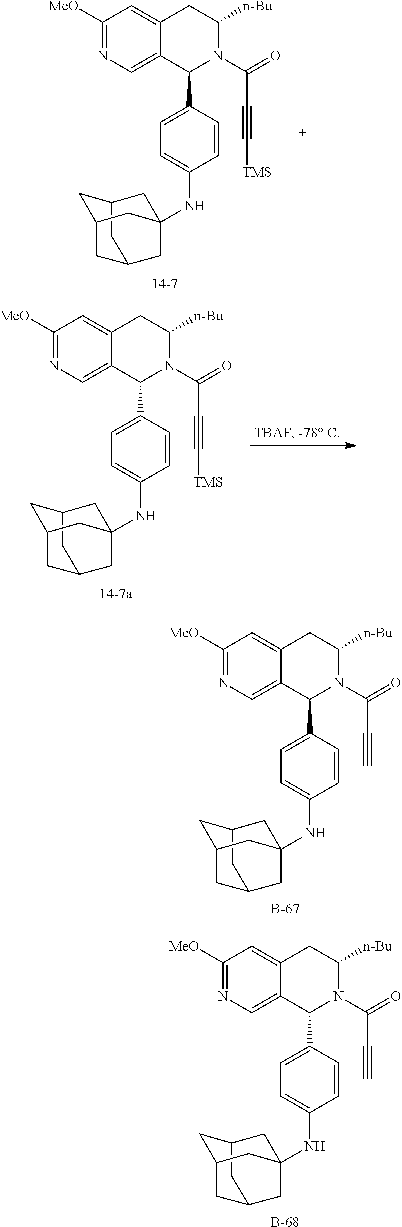

### General Procedure for the Preparation of Compound 14-2

To a solution of 14-1 (5.05 g, 26.85 mmol, 12.49 mL, 1.5 eq) in THF (50 mL) was added n-BuLi (2.5 M, 11.46 mL, 1.6 eq) at −78° C. under N₂. The mixture was stirred for 1 h at this temperature to give a brown solution. Then Bu-3 (5 g, 17.90 mmol, 1 eq) in THF (10 mL) was added to the mixture at −78° C. The result mixture was allowed and stirred at 20° C. for 12 h to give a red solution. TLC (PE/EtOAc=4/1) showed the reaction was completed. The reaction was quenched with saturated citric acid (100 mL) and stirred at 20° C. for 0.5 hr. It was extracted with EtOAc (80 mL*2). The organic layers were adjusted to pH=8 with saturated NaHCO₃ (200 mL), extracted with EtOAc (100 mL*2). The organic layers were dried over sodium sulfate, filtered and concentrated under vacuum. The residue was purified by column chromatography (SiO₂, Petroleum ether/Ethyl acetate=100/0 to 92/8) to give 14-2 (2.01 g, 6.52 mmol, 36.41% yield).

### General Procedure for the Preparation of Compound 14-3

A solution of 14-2 (2.05 g, 6.65 mmol, 1 eq) in HCl/MeOH (4 M, 20 mL, 12.04 eq). The mixture was stirred at 20° C. for 2 hr to give a yellow solution. LCMS showed the mixture was completed. The mixture was concentrated under vacuum. The oil was triturated with MTBE (15 mL*3). The filter cake was diluted with MeOH (10 mL) and concentrated under vacuum. The product was used next step without further purification. 14-3 (1.96 g, 4.18 mmol, 62.89% yield).

### General Procedure for the Preparation of Compound 14-4

To a solution of 4-(1-adamantylamino)benzoic acid (500 mg, 1.84 mmol, 1 eq) in DCM (10 mL) was added DIEA (1.19 g, 9.21 mmol, 1.60 mL, 5 eq) and HATU (735.65 mg, 1.93 mmol, 1.05 eq) at 10° C. for 1 hr to give a green solution. LCMS showed 4-(1-adamantylamino)benzoic acid was consumed and desired mass=390 was found. Then it was added 14-3 (888.31 mg, 2.21 mmol, 1.2 eq). The mixture was stirred at 50° C. for 12 hr to give a green solution. LCMS and TLC (PE:EtOAc=2:1) showed the mixture was completed. The reaction mixture was quenched with H₂O (20 mL) and extracted with DCM (15 mL*3). The organic layers were dried over Na₂SO₄, filtered and concentrated to give the crude product. The residue was purified by column chromatography (SiO₂, Petroleum ether/Ethyl acetate=100/0 to 77/23 to 65/35) to give 14-4 (472 mg, 1.02 mmol, 55.49% yield).

### General Procedure for the Preparation of Compound 14-5

A solution of 14-4 (600 mg, 1.30 mmol, 1 eq) in POCl₃ (19.80 g, 129.13 mmol, 12 mL, 99.35 eq) pre-heated to 140° C. The mixture was stirred at 140° C. for 3 hr to give a red solution. LCMS and TLC (PE:EtOAc=0:1) showed the mixture was completed. The mixture was concentrated under reduced pressure, the residue was dissolved in EtOAc (30 mL), the mixture was adjusted pH=8˜9 by Na₂CO₃ (saturated, aq). The resulting mixture was separated. The aqueous phase was extracted by EtOAc (30 mL*2). The combined organic layers were combined and dried over anhydrous sodium sulfate and concentrated. The residue was purified by column chromatography (SiO₂, Petroleum ether/Ethyl acetate=100/0 to 75/25) to give impure product (150 mg). The residue was purified by prep-TLC (SiO₂, Petroleum ether/Ethyl acetate=2:1) to give 14-5 (60 mg, 135.25 μmol, 10.41% yield).

### General Procedure for the Preparation of Compound 14-6 & 14-6a

To a solution of 14-5 (65.8 mg, 148.32 μmol, 1 eq) in MeOH (5 mL) pre-heated to 50° C. were added NaBH₄ (28.06 mg, 741.62 μmol, 5 eq). The mixture was stirred at 50° C. for 10 mins. Then it was added NaBH₄ (28.06 mg, 741.62 μmol, 5 eq). The mixture was stirred at 50° C. for 1 hr to give a clear solution. LCMS and TLC (PE:EtOAc=1:3) showed the mixture was completed. The mixture was quenched with NaHCO₃ (5 mL), extracted with EtOAc (5 mL*3). The organic layers were dried over sodium sulfate, filtered and concentrated under vacuum. The residue was purified by column chromatography by prep-TLC (SiO₂, Petroleum ether/Ethyl acetate=1:1) to give 14-6 (4 mg, 8.98 μmol, 6.05% yield) and 14-6a (8 mg, 17.95 μmol, 12.10% yield).

### General Procedure for the Preparation of Compound 14-7

To a solution of 3-trimethylsilylprop-2-ynoic acid (7.98 mg, 56.10 μmol, 1 eq) in DCM (2 mL) was added 2-chloro-1-methyl-pyridin-1-ium; iodide (14.33 mg, 56.10 μmol, 1 eq). The mixture was stirred at 20° C. for 1 hr to give a yellow suspension. The result mixture was dropwise to a solution of 14-6 (25 mg, 56.10 μmol, 1 eq) and TEA (5.68 mg, 56.10 μmol, 7.81 μL, 1 eq) in DCM (2 mL) at 0° C. It was stirred at 0° C. for 0.5 hr to give a yellow solution. LCMS showed the mixture was completed. The mixture was quenched with aq·NH₄Cl (10 mL), extracted with DCM (10 mL*3). The organic layer was separated and dried over sodium sulfate, filtered and concentrated under vacuum. Used next step without further purification. 14-7 (45 mg, crude).

### General Procedure for the Preparation of Compound B-67

To a solution of 14-7 (45.00 mg, 78.97 μmol, 1 eq) in THF (5 mL) were added TBAF (1 M, 86.86 μL, 1.1 eq) at −78° C. The mixture was stirred at −78° C. for 20 mins to give a clear solution. LCMS and TLC (PE:EtOAc=3:2) showed the mixture was completed. The mixture was added H₂O (8 mL), extracted with EtOAc (8 mL*3). The organic layers were dried over sodium sulfate, filtered and concentrated under vacuum. The residue was purified by column chromatography (SiO₂, Petroleum ether/Ethyl acetate=100/0 to 74/26) to give impure product. Then it was purified by prep-TLC (SiO₂, Petroleum ether/Ethyl acetate=2:1) to give B-67 (10.1 mg, 19.88 μmol, 25.18% yield). LC-MS (m/z): 498.3 [M+H]⁺. ¹H NMR (400 MHz, CDCl₃) δ ppm 8.25-8.02 (m, 1H) 6.97-6.80 (m, 2H) 6.71-6.60 (m, 2H) 6.56 (s, 1H) 6.30 (d, J=6.8 Hz, 1H) 4.71-4.42 (m, 1H) 3.95 (d, J=7.6 Hz, 3H) 3.20-2.66 (m, 3H) 2.15-2.00 (m, 3H) 1.91-1.80 (m, 6H) 1.70-1.62 (m, 6H) 1.35-1.06 (m, 6H) 0.90-0.82 (m, 3H).

### General Procedure for the Preparation of Compound 14-7a

To a solution of 3-trimethylsilylprop-2-ynoic acid (22.34 mg, 157.08 μmol, 1 eq) in DCM (2 mL) was added 2-chloro-1-methyl-pyridin-1-ium; iodide (40.13 mg, 157.08 μmol, 1 eq). The mixture was stirred at 20° C. for 1 hr to give a yellow suspension. The result mixture was dropwise to a solution of 14-6a (70 mg, 157.08 μmol, 1 eq) and TEA (15.89 mg, 157.08 μmol, 21.86 μL, 1 eq) in DCM (3 mL) at 0° C. It was stirred at 0° C. for 0.5 hr to give a yellow solution. LCMS and TLC (PE:EtOAc=2:1) showed the mixture was completed. The mixture was quenched with aq·NH₄Cl (10 mL), extracted with DCM (10 mL*3). The organic layer was separated and dried over sodium sulfate, filtered and concentrated under vacuum. The residue was purified by column chromatography (SiO₂, Petroleum ether/Ethyl acetate=100/0 to 80/20) to give 14-7a (60 mg, 105.29 μmol, 67.03% yield).

### General Procedure for the Preparation of Compound B-68

To a solution of 14-7 (60 mg, 105.29 μmol, 1 eq) in THF (5 mL) were added TBAF (1 M, 115.82 μL μL, 1.1 eq) at −78° C. The mixture was stirred at −78° C. for 20 mins to give a clear solution. LCMS and TLC (PE:EtOAc=3:2) showed the mixture was completed. The mixture was added H₂O (8 mL), extracted with EtOAc (8 mL*3). The organic layers were dried over sodium sulfate, filtered and concentrated under vacuum. The residue was purified by column chromatography (SiO₂, petroleum ether/ethyl acetate=100/0 to 74/26) to give B-68 (33.58 mg, 65.72 μmol, 62.42% yield). LC-MS (m/z): 498.2 [M+H]⁺. ¹H NMR (400 MHz, CDCl₃) δ ppm 8.13-7.85 (m, 1H) 7.10-6.99 (m, 1H) 6.93 (d, J=8.4 Hz, 1H) 6.85-6.56 (m, 4H) 4.70-4.12 (m, 1H) 3.96 (d, J=9.2 Hz, 3H) 3.17 (d, J=2.8 Hz, 1H) 3.13-2.85 (m, 1H) 2.77-2.37 (m, 1H) 2.11 (br s, 3H) 1.92-1.83 (m, 6H) 1.72-1.63 (m, 6H) 1.33-0.92 (m, 6H) 0.83-0.69 (m, 3H).

### Procedure 57: Compound B-69

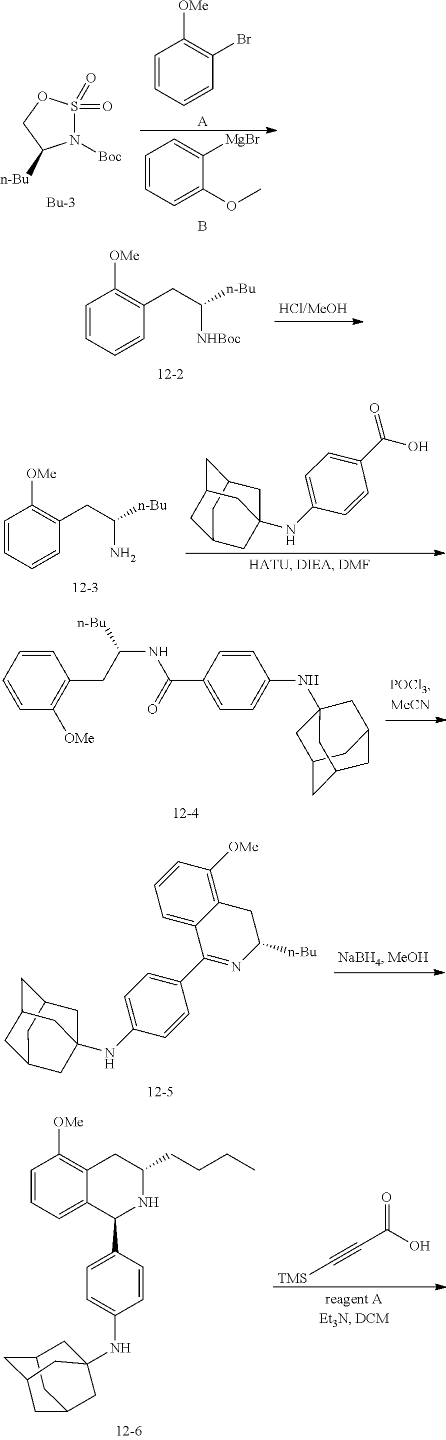

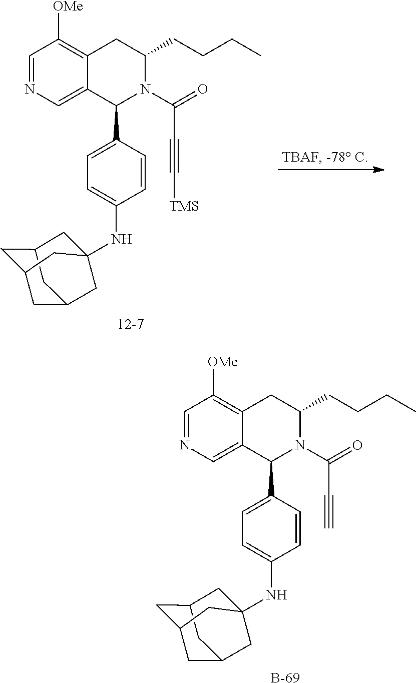

### General Procedure for the Preparation of Compound 12-2

To a solution of CuI (40.91 mg, 214.78 μmol, 0.012 eq) was placed in a was added bromo-(2-methoxyphenyl)magnesium (1.0 M, 21.48 mL, 1.2 eq) at 0° C. under N₂. The reaction was stirred 0° C. under N₂ for 2 hr. Then Bu-3 (5 g, 17.90 mmol, 1 eq) in THF (50 mL) was added dropwise at −20° C. The reaction was allowed to stir at 20° C. for 12 hr to give a yellow solution. TLC (PE/EtOAc=5:1) showed the reaction was completed. The reaction was quenched with saturated citric acid (50 mL) and extracted with EtOAc (50 mL*3). The organic layers were dried over Na₂SO₄ and concentrated to give the crude product. The residue was purified by column chromatography (SiO₂, Petroleum ether/Ethyl acetate=1/0 to 97/3) to give 12-2 (1.87 g, 6.08 mmol, 33.97% yield).

### General Procedure for the Preparation of Compound 12-3

To solution of 12-2 (1.87 g, 6.08 mmol, 1 eq) was added HCl/dioxane (4 M, 63.39 mL, 41.68 eq), The mixture was stirred at 15° C. for 12 hr to give a yellow suspension. TLC (PE/EtOAc=5:1) showed the reaction was completed. The mixture was concentrated to give 12-3 (1.66 g, crude, HCl) which was used for next step without further purification.

### General Procedure for the Preparation of Compound 12-4

To a solution of 4-(1-adamantylamino)benzoic acid (1.81 g, 6.68 mmol, 1.1 eq) in DCM (15 mL) was added HATU (2.77 g, 7.29 mmol, 1.2 eq) and DIEA (2.35 g, 18.21 mmol, 3.17 mL, 3 eq). The mixture was stirred at 20° C. for 1 hr. Then 12-3 (1.48 g, 6.07 mmol, 1 eq, HCl) in DCM (15 mL) was added. The reaction was stirred at 20° C. for 12 hr to give a yellow solution. LCMS showed the reaction was completed. The reaction mixture was quenched with H₂O (30 mL) and extracted with DCM (30 mL*3). The organic layers were dried over Na₂SO₄ and concentrated to give the crude product. The residue was purified by column chromatography (SiO₂, Petroleum ether/Ethyl acetate=100/0 to 69/31) to give 12-4 (1.98 g, 4.30 mmol, 70.80% yield).

### General Procedure for the Preparation of Compound 12-5

To a solution of 12-4 (1.98 g, 4.30 mmol, 1 eq) in DCM (20 mL) was added 2-chloropyridine (1.46 g, 12.89 mmol, 1.22 mL, 3 eq) and Tf₂O (3.64 g, 12.89 mmol, 2.13 mL, 3 eq) at −78° C. under N₂. After 15 min, the reaction mixture was placed in an ice-water bath and warmed to 0° C. After 15 min, the resulting solution was allowed to warm to 20° C. The reaction was allowed to stir at 20° C. under N₂ for 1 hr, but LCMS showed the reaction was not completed. Then the reaction was stir at 20° C. under N₂ for 12 hr to give a yellow solution. LCMS showed the reaction was completed. The mixture was quenched with saturated NaHCO₃ (15 mL), extracted with DCM (15 mL*3). The organic layers were dried over sodium sulfate, filtered and concentrated under vacuum. The residue was purified by column chromatography (SiO₂, Petroleum ether/Ethyl acetate=100/0 to 60/40) to give 12-5 (1.65 g, 3.73 mmol, 86.72% yield).

### General Procedure for the Preparation of 12-6

To a solution of 12-5 (800 mg, 1.81 mmol, 1 eq) in MeOH (15 mL) was added NaBH₄ (341.86 mg, 9.04 mmol, 5 eq) at 50° C. The reaction was stirred 1 hr to give a yellow solution. LCMS showed the reaction was completed. The mixture was added NaHCO₃ (20 mL) and filtered. Then extracted with EtOAc (20 mL*3). The organic layers were dried over Na₂SO₄, filtered and concentrated under vacuum. The crude product was purified by column chromatography (SiO₂, Petroleum ether/Ethyl acetate=100/0 to 81/19) and prep-TLC (SiO₂, Petroleum ether/Ethyl acetate=1/1) to give 12-6 (80 mg, 179.92 μmol, 4.98% yield).

### General Procedure for the Preparation of 12-7

To a solution of 3-trimethylsilylprop-2-ynoic acid (25.59 mg, 179.92 μmol, 1 eq) in DCM (2 mL) was added 2-chloro-1-methyl-pyridin-1-ium; iodide (45.97 mg, 179.92 μmol, 1 eq). The reaction was stirred at 20° C. for 0.5 hr. Then the mixture was added dropwise in solution of 12-6 (80 mg, 179.92 μmol, 1 eq) and TEA (36.41 mg, 359.83 μmol, 50.08 μL, 2 eq) in DCM (2 mL) at 0° C. The reaction was stirred at 0° C. for 1 hr to give a yellow solution. LCMS showed the reaction was completed. The reaction mixture was washed with saturated NH₄Cl (10 mL) and extracted with DCM (10 mL*3). The organic layers were dried over Na₂SO₄ and concentrated to give 12-7 (100 mg, 175.79 μmol, 97.71% yield) which was used for next step without further purification.

### General Procedure for the Preparation of B-69

To a solution of 12-7 (100 mg, 175.79 μmol, 1 eq) in THF (2 mL) was added TBAF (1 M, 193.37 μL, 1.1 eq) at −78° C. The reaction was stirred at −78° C. for 15 min to give a yellow solution. LCMS showed the reaction was completed. The reaction mixture was quenched H₂O (10 mL) and extracted with EtOAc (10 mL*3). The organic layers were dried over Na₂SO₄ and concentrated to give the crude product. The residue was purified by column chromatography (SiO2, Petroleum ether/Ethyl acetate=100/0 to 75/25) to give B-69 (33.6 mg, 65.92 μmol, 37.50% yield). LC-MS (m/z): 497.3[M+H]⁺. ¹H NMR (CDCl₃, 400 MHz): δ=7.25-7.15 (m, 1H), 7.02-6.87 (m, 3H), 6.80 (br dd, J=15.7, 8.2 Hz, 1H), 6.65 (br s, 2H), 6.34-6.14 (m, 1H), 4.69-4.36 (m, 1H), 3.81 (br s, 3H), 3.38-3.16 (m, 1H), 3.15-2.89 (m, 1H), 2.80-2.37 (m, 2H), 2.07 (br s, 3H), 1.82 (br d, J=11.5 Hz, 6H), 1.63 (br s, 6H), 1.34-0.92 (m, 6H), 0.82 ppm (br d, J=7.5 Hz, 3H).

### Procedure 58: Compound B-70

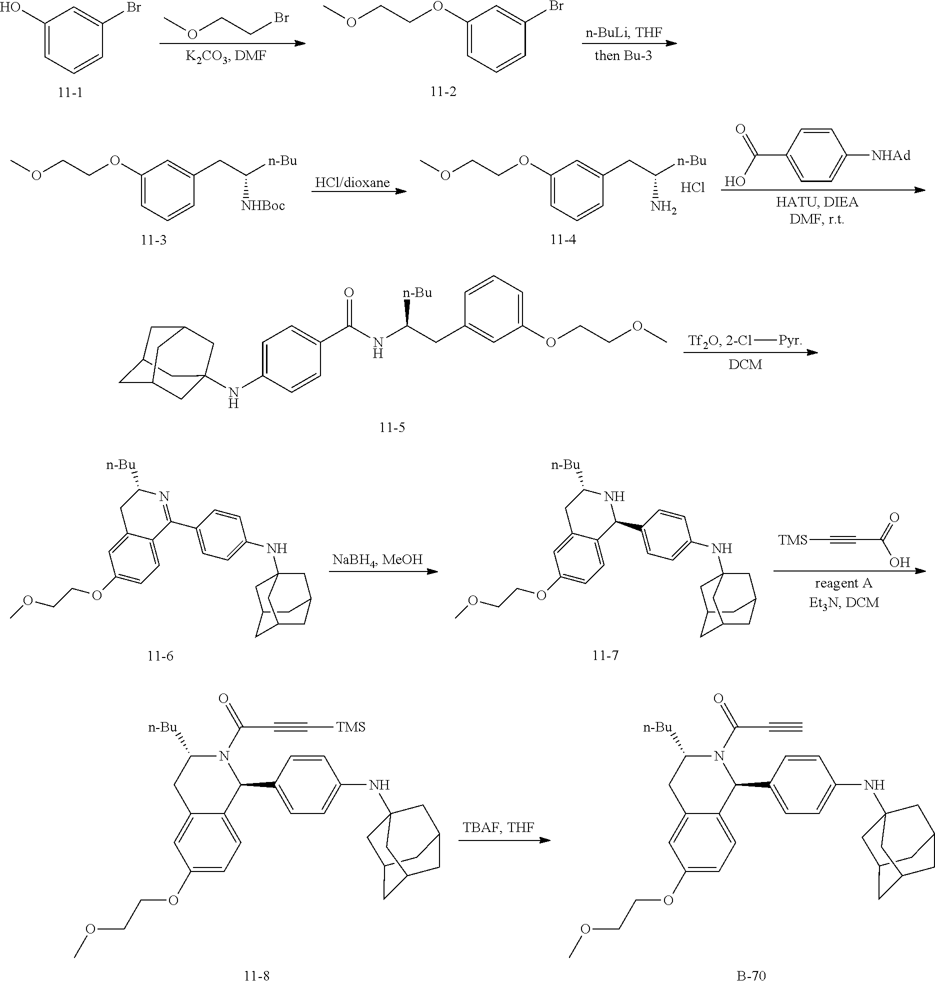

### General Procedure for the Preparation of Compound 11-2

To a solution of 11-1 (10 g, 57.80 mmol, 1 eq) in DMF (120 mL) was added K₂CO₃ (23.97 g, 173.40 mmol, 3 eq) and 1-bromo-2-methoxy-ethane (8.84 g, 63.58 mmol, 5.97 mL, 1.1 eq), the resulting mixture was stirred at 20° C. for 12 h. TLC (PE/EtOAc=8/1) showed the reaction was completed. The reaction mixture was quenched with H₂O (250 mL) and extracted with EtOAc (300 mL*3). The combined organic layers were washed with saturated NaCl (200 mL), dried over Na₂SO₄, filtered and concentrated to give the crude product. The crude product was purified by flash column (SiO₂, PE/EtOAc=1/0-97/3) to afford 11-2 (8.2 g, 35.48 mmol, 61.39% yield). ¹H NMR (400 MHz, chloroform-d) δ=7.14-7.06 (m, 3H), 6.84-6.84 (m, 1H), 4.09 (t, 2H), 3.73 (t, 2H), 3.44 (s, 3H).

### General Procedure for the Preparation of Compound 11-3

To a solution of 11-2 (5 g, 21.64 mmol, 1.5 eq) in THF (50 mL) was added n-BuLi (2.5 M, 9.23 mL, 1.6 eq) at −78° C. under N₂. The reaction was stirred −78° C. under N₂ for 0.5 h. Then Bu-3 (4.03 g, 14.42 mmol, 1 eq) in THF (40 mL) was added dropwise at −78° C. under N₂. The reaction was stirred at 15° C. for 11.5 h to give a yellow solution. TLC (PE/EtOAc=8/1) showed the reaction was completed. The reaction was quenched with saturated citric acid (8 mL) and the mixture was stirred for another 30 min. The mixture was extracted with EtOAc (150 mL*3). The combined organic layers were dried over Na₂SO₄ and concentrated to give the crude product. The crude product was purified by flash column (SiO₂, PE/EtOAc=1/0 to 96/4) to afford 11-3 (1.61 g, 4.47 mmol, 31.01% yield). ¹H NMR (400 MHz, chloroform-d) δ=7.19-7.16 (t, 1H), 6.78-6.75 (m, 3H), 4.55-4.51 (m, 1H), 4.21-4.17 (m, 1H), 3.75-3.73 (t, 3H), 3.45 (s, 3H), 2.72-2.70 (t, 3H), 1.47-1.36 (m, 16H), 0.94-0.90 (t, 3H).

### General Procedure for the Preparation of Compound 11-4

11-3 (0.36 g, 1.02 mmol, 1 eq) was dissolved in HCl/dioxane (4 M, 4.01 mL, 15.67 eq), the mixture was stirred at 20° C. for 12 h to give a yellow solution. LCMS and TLC (PE/EtOAc=8:1) showed the reaction was completed. The mixture was concentrated to afford the crude product 11-4 (0.23 g, crude, HCl). Used directly in next step without further purification. LC-MS (m/z): 252.0 [M+H]⁺. ¹H NMR (400 MHz, chloroform-d) δ=7.27 (t, 1H), 6.89-6.84 (m, 3H), 4.13-4.11 (t, 2H), 3.69-3.59 (m, 3H), 3.42 (s, 3H), 1.62-1.60 (m, 2H), 1.40-1.34 (m, 4H), 0.94-0.90 (t, 3H).

### General Procedure for the Preparation of Compound 11-5

To a solution of 11-4 (1.46 g, 5.07 mmol, 1 eq, HCl) in DMF (14 mL) were 4-(1-adamantylamino)benzoic acid (1.38 g, 5.07 mmol, 1 eq), DIEA (1.31 g, 10.14 mmol, 1.77 mL, 2 eq) and HATU (3.86 g, 10.14 mmol, 2 eq). The mixture was stirred at 20° C. for 12 h to give a black solution. TLC (PE/EtOAc=2/1) showed the reaction was completed. The mixture was quenched with NaHCO₃ (30 mL) and extracted with EtOAc (60 mL*3). The combined organic layers were dried over Na₂SO₄, filtrated and concentrated to give the crude product. The crude product was purified by silica column (SiO₂, PE/EtOAc=1/0 to 70/30) to afford 11-5 (1.25 g). LC-MS (m/z): 505.0 [M+H]⁺. ¹H NMR (400 MHz, CHLOROFORM-d) δ=7.50-7.48 (d, 2H), 7.20-7.16 (t, 1H), 6.80-6.77 (m, 3H), 6.69-6.67 (d, 2H), 4.34-4.33 (m, 1H), 4.07-4.05 (m, 2H), 3.72-3.69 (t, 2H), 3.42 (s, 3H), 2.12 (s, 3H), 1.96-1.93 (s, 6H), 1.72-1.65 (m, 8H), 1.36-1.30 (m, 6H), 0.86-0.83 (t, 3H).

### General Procedure for the Preparation of Compound 11-6

To a solution of 11-5 (0.2 g, 396.27 μmol, 1 eq) in DCM (10 mL) were added 2-CHLOROPYRIDINE (134.98 mg, 1.19 mmol, 112.49 μL, 3 eq) and Tf₂O (335.41 mg, 1.19 mmol, 196.15 μL, 3 eq) at −78° C. under N₂. After 15 min, the reaction mixture was placed in an ice-water bath and warmed to 0° C. After 15 min, the resulting solution was allowed to warm to 20° C. The reaction was stirred at 20° C. under N₂ for 1 h to give a deep purple solution. LCMS and TLC (PE/EtOAc=0/1) showed the mixture was completed. The mixture was quenched with saturated NaHCO₃ (20 mL) and extracted with DCM (25 mL*3). The organic layers were Na₂SO₄, filtered and concentrated to give the crude product. The crude product was purified by flash column (SiO₂, PE/EtOAc=100/0 to 15/85) to give 11-6 (0.08 g, 164.38 μmol, 41.48% yield). LC-MS (m/z): 487.0 [M+H]⁺. ¹H NMR (400 MHz, CHLOROFORM-d) δ=7.51-7.47 (t, 3H), 6.89-6.87 (d, 2H), 6.80-6.78 (d, 2H), 4.24-4.21 (t, 2H), 3.97 (s, 2H), 3.46 (s, 3H), 3.14-3.10 (m, 1H), 2.95 (s, 1H), 2.88 (s, 1H), 2.15 (s, 1H), 1.99 (s, 6H), 1.88-1.80 (m, 1H), 1.71-1.68 (m, 7H), 1.44-1.32 (m, 4H), 0.91-0.87 (t, 3H).

### General Procedure for the Preparation of Compound 11-7

To a solution of 11-6 (330 mg, 678.05 μmol, 1 eq) in MeOH (4 mL) was added NaBH₄ (128.26 mg, 3.39 mmol, 5 eq). The reaction was stirred at 20° C. for 1 hr to give yellow solution. LCMS showed the reaction was completed. The reaction mixture was poured into saturated NaHCO₃ aq. (10 mL) and extracted with EtOAc (10 mL×3). The combined organic layers were washed with saturated NaCl aq. and dried over Na₂SO₄, filtered and concentrated under reduced pressure. The crude products were combined for further purification. The combined crude product was purified by flash column (SiO₂, eluting with: PE/EtOAc=0% to 50%) to give 11-7 (200 mg, 409.25 μmol, 60.36% yield).

### General Procedure for the Preparation of Compound 11-8

To a solution of 3-trimethylsilylprop-2-ynoic acid (10.48 mg, 73.66 μmol, 1.2 eq) in DCM (0.5 mL) were added 2-chloro-1-methyl-pyridin-1-ium; iodide (15.68 mg, 61.39 μmol, 1 eq). The mixture was stirred at 20° C. for 0.5 hr to give a yellow suspension. Then a solution of 11-7 (30 mg, 61.39 μmol, 1 eq) and TEA (6.21 mg, 61.39 μmol, 8.54 μL, 1 eq) in DCM (0.5 mL) was dropwise. It was stirred at 0° C. for 2 hr to give a yellow solution. TLC (PE/EtOAc=0/1) showed the reaction was completed. The reaction was basified with saturated NH₄Cl aq. (5 mL), extracted with DCM (5 mL×3). The combined organic layers were dried over Na₂SO₄, filtered and concentrated under reduced pressure. The crude products were combined for further purification. The residue was purified by prep-TLC (PE/EtOAc=1/2) to give 11-8 (40 mg).

### General Procedure for the Preparation of Compound B-70

To a solution of 11-8 (40 mg, 65.26 μmol, 1 eq) in THF (1 mL) was added TBAF (1.0 M, 71.79 μL, 1.1 eq) at −78° C. The reaction was allowed to stir at −78° C. for 0.5 hr to give a yellow solution. TLC (PE/EtOAc=2/1) showed the reaction was completed. The reaction mixture was poured into H2O (5 mL) and extracted with EtOAc (5 mL×3). The combined organic layers were dried over Na₂SO₄, filtered and concentrated under reduced pressure. The crude product was purified by flash column (SiO₂, eluting with: PE/EtOAc=0% to 40%) to give B-70 (19.11 mg, 32.55 μmol, 49.87% yield). LC-MS (m/z): 541.2 [M+H]⁺. ¹H NMR (400 MHz, CHLOROFORM-d) δ ppm 7.22 (d, J=8.53 Hz, 1H) 7.16-6.78 (m, 4H) 6.74 (s, 1H) 6.66 (br s, 1H) 6.24 (d, J=14.56 Hz, 1H) 4.68-4.42 (m, 1H) 4.12 (q, J=4.94 Hz, 2H) 3.76 (dt, J=5.83, 2.98 Hz, 2H) 3.46 (d, J=2.01 Hz, 3H) 3.23-2.63 (m, 3H) 2.09-1.96 (m, 3H) 1.87-1.77 (m, 6H) 1.61 (br s, 6H) 1.35-1.11 (m, 6H) 0.87-0.82 (m, 3H).

### Procedure 59: Compound B-72

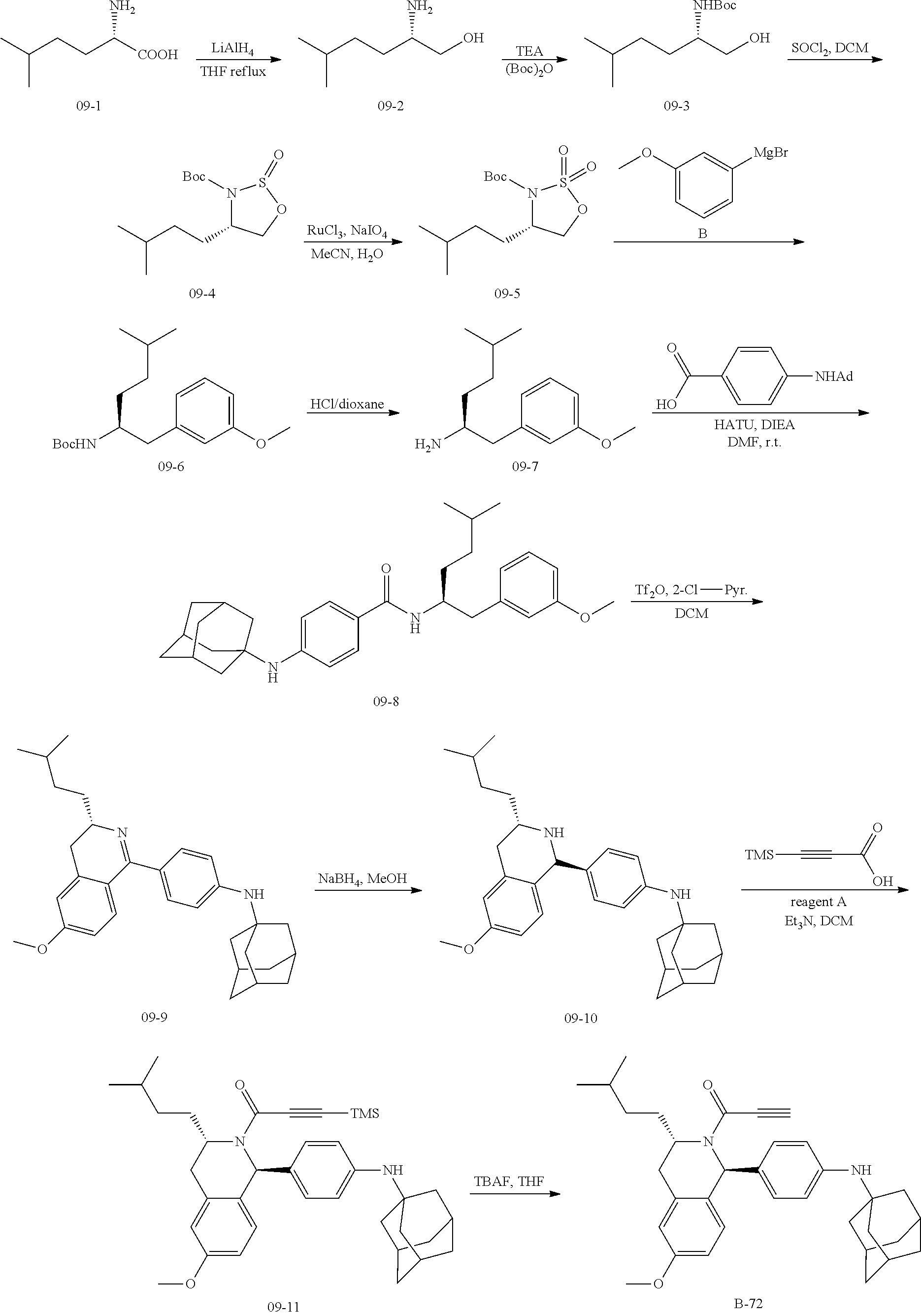

### General Procedure for the Preparation of 09-2

To a solution of LAH (5.49 g, 144.63 m mol, 1.2 eq.) in THF (400 mL) was added 09-1 (17.5 g, 120.52 m mol, 1 eq.) at 0° C. The mixture was warmed to 25° C. and stirred at 70° C. for 12 h to give a white suspension. The reaction was quenched with H₂O (20 mL), 15% NaOH (20 mL) and H₂O (60 mL), diluted with THF (60 mL) and dried over sodium sulfate, filtered and concentrated to give (2S)-2-amino-5-methyl-hexan-1-ol (12.31 g, 93.82 m mol, 77.84% yield). Used for next further without further purification. Compound 09-2 (12.31 g, 93.82 m mol, 77.84% yield). ¹H NMR (400 MHz, CDCl₃) δ=3.57 (m, 1H), 3.25 (m, 1H), 2.76 (m, 1H), 1.97 (s, 2H), 1.53 (m, 2H), 1.22 (m, 2H), 0.88 (t, J=6.8 Hz, 6H).

### General Procedure for the Preparation of 09-3

To a solution of 09-2 (12.31 g, 93.82 m mol, 1 eq.) in DCM (130 mL) was added TEA (14.24 g, 140.72 m mol, 19.59 mL, 1.5 eq.) and tert-butoxycarbonyl tert-butyl carbonate (18.43 g, 84.43 m mol, 19.40 mL, 0.9 eq.) at 0° C. The mixture was warmed to 20° C. and stirred for 12 h to give a yellow clear solution. TLC (PE:EA=3:1) showed the reaction was completed. The reaction was quenched with H₂O (40 mL). The separated aqueous layer was extracted with DCM (40 mL×4). The combined organic layers were washed with brine (40*2 mL) and dried over sodium sulfate, filtered and concentrated to give the crude product. The crude product was purified by silica column (SiO₂, PE to PE:EA=10:1). Compound 09-3 (5.55 g, 23.99 m mol, 25.57% yield). ¹H NMR (400 MHz, CDCl₃) δ=4.59 (s, 1H), 3.6 (m, 3H), 2.42 (s, 1H), 1.64 (s, 1H), 1.46 (m, 10H), 1.24 (m, 2H), 0.88 (t, J=6.4 Hz, 6H).

### General Procedure for the Preparation of 09-4

A solution of imidazole (7.85 g, 115.26 m mol, 5.86 eq.) in DCM (70 mL) was cooled to 0° C. SOCl₂ (4.07 g, 34.22 m mol, 2.48 mL, 1.74 eq.) in DCM (7 mL) was added to the solution at 0° C. The result suspension was stirred at 20° C. for 1 h. Then the mixture was cooled to −78° C., a solution of tert-butyl N-[(1S)-1-(hydroxyethyl)-4-methyl-pentyl]carbamate (4.55 g, 19.67 m mol, 1 eq.) in DCM (49 mL) was added to the mixture dropwise. The result mixture was allowed to 20° C. and stirred for 12 h to give a yellow solution. TLC (PE/EA=3:1) showed the reaction was completed. The mixture was filtered and the filtrate was washed with H₂O (40 mL), extracted with DCM (40 mL*3). The organic layers were dried over sodium sulfate and filtered Compound 09-4 (6.95 g, 25.06 m mol, 63.69% yield). ¹H NMR (400 MHz, CDCl₃) δ=4.96 (m, 1H), 4.75 (m, 1H), 4.41 (s, 1H), 1.64 (t, J=9.2 Hz, 1H), 4.03 (m, 2H), 1.68 (m, 1H), 1.52 (s, 9H), 1.44 (s, 3H), 1.23 (m, 2H), 0.89 (t, J=6.8 Hz, 6H).

### General Procedure for the Preparation of 09-5

RuCl₃0.3H2O (32.76 mg, 125.28 u mol, 0.005 eq.) was added to a stirred mixture of tert-butyl (4S)-4-isopentyl-2-oxo-oxathiazolidine-3-carboxylate (6.95 g, 25.06 m mol, 1 eq.) in MeCN (70 mL) and H2O (45 mL) at 0° C., followed by portion wise addition of NaIO₄ (5.90 g, 27.56 m mol, 1.53 mL, 1.1 eq.) The mixture was allowed to warm to 20° C. and stirred for 12 h to give a black suspension. TLC (PE/EA=5:1) showed the reaction was completed. The reaction was filtered through celite and washed with EA (30 mL). The filtrate was extracted with EA. (30 mL*3). The combined organic layers were washed with brine (30 mL*2) and dried over sodium sulfate, filtered and concentrated to give the crude product. The crude product was purified by silica column (SiO₂, PE. to PE:EA=30:1). Compound 09-5 (2.10 g, 7.16 mmol, 28.57% yield). ¹H NMR (400 MHz, CDCl₃) δ=4.63 (t, J=8.8 Hz, 1H), 4.29 (m, 2H), 1.55 (m, 11H), 1.22 (m, 3H), 0.91 (t, J=6.8 Hz, 6H).

### General Procedure for the Preparation of Compound 09-6

To a solution of CuI (8.57 mg, 44.99 μmol, 0.012 eq) was placed in a was added bromo-(3-methoxyphenyl)magnesium (1 M, 4.50 mL, 1.2 eq) at 0° C. under N2. The reaction was stirred 0° C. under N2 for 2 hr. Then 10-5 (1.1 g, 3.75 mmol, 1 eq) in THF (10 mL) was added dropwise at −50° C. The reaction was allowed to stir at 20° C. for 12 h to give a yellow solution. TLC (eluting with: PE/EtOAc=5/1) showed the reaction was completed. The reaction was quenched with saturated citric acid (10 mL) and extracted with EtOAc (20 mL*3). The organic layers were dried over Na2SO₄ and concentrated to give the crude product. The crude product was purified by flash column (eluting with: PE/EtOAc=0% to 3%) to give 09-6 (590 mg, 1.84 mmol, 48.95% yield).

### General Procedure for the Preparation of Compound 09-7

The 09-6 (590 mg, 1.84 mmol, 1 eq) was dissolved in HCl/EtOAc (4 M, 10 mL, 21.79 eq), the mixture was stirred at 20° C. for 12 h to give a yellow suspension. LCMS showed the desired MS. The mixture was concentrated under reduced pressure to afford the crude product. The product was used for next step without further purification to give 09-7 (480 mg, crude, HCl).

### General Procedure for the Preparation of Compound 09-8

To a solution of 4-(1-adamantylamino)benzoic acid (808.14 mg, 2.98 mmol, 1.1 eq) in DCM (10 mL) was added HATU (1.24 g, 3.25 mmol, 1.2 eq) and DIEA (1.05 g, 8.12 mmol, 1.41 mL, 3 eq). The mixture was stirred at 20° C. for 1 h. Then 09-7 was added. The reaction was stirred at 20° C. for 15 h to give a yellow solution. LCMS showed the desired MS. The mixture was concentrated under reduced pressure to afford the crude product. The crude product was purified by flash column (eluting with: PE/EtOAc=0% to 20%) to give 09-8 (1 g, 2.17 mmol, 80.18% yield).

### General Procedure for the Preparation of Compound 09-9

To a solution of 09-8 (900 mg, 1.90 mmol, 1 eq) in DCM (20 mL) was added 2-chloropyridine (645.85 mg, 5.69 mmol, 538.21 μL, 3 eq) and Tf2O (1.60 g, 5.69 mmol, 938.50 μL, 3 eq) at −78° C. under nitrogen atmosphere. The mixture was stirred at −78° C. for 15 min. Then the mixture was stirred at −0° C. for 15 min. The mixture was stirred at 20° C. for 1 h to give a yellow solution. LCMS showed the desired MS. The reaction mixture was poured into water (10 mL) and extracted with DCM (20 mL×2). The combined organic layers were washed with saturated Na₂CO₃ aq. (10 mL×2), dried over Na₂SO₄, filtered and concentrated under reduced pressure. The crude product was purified by flash column (eluting with: PE/EtOAc=0% to 40%) to give 09-9 (770 mg, 1.69 mmol, 88.93% yield).

### General Procedure for the Preparation of Compound 09-10

To a solution of 09-9 (320 mg, 700.74 μmol, 1 eq) in MeOH (5 mL) was added NaBH₄ (132.55 mg, 3.50 mmol, 5 eq) at 50° C. The mixture was stirred at 50° C. for 1 h to give a yellow solution. LCMS indicated the product MS value was observed. The reaction was quenched by NH₄Cl (saturated, aq., 30 mL). The resulting mixture was extracted by EtOAc (30 mL*2). The combined organic layers were washed by brine (30 mL*2), the organic layer was dried over anhydrous sodium sulfate, filtered and concentrated under reduced pressure to afford the crude product. The crude product was purified by prep-TLC (petroleum ether:EtOAc=0:1) to afford 09-10 (27.8 mg, 60.61 μmol, 8.65% yield) and N-[4-[(1R,3S)-3-isopentyl-6-methoxy-1,2,3,4-tetrahydroisoquinolin-1-yl]phenyl]adamantan-1-amine (130 mg, 283.42 μmol, 40.45% yield).

### General Procedure for the Preparation of Compound 09-11

To a solution of 3-trimethylsilylprop-2-ynoic acid (8.62 mg, 60.61 μmol, 1 eq) and 2-chloro-1-methyl-pyridin-1-ium; iodide (15.48 mg, 60.61 μmol, 1 eq) in DCM (3 mL). The mixture was stirred at 20° C. for 30 min. Then the mixture was dropwise to a solution of 09-10 (27.8 mg, 60.61 μmol, 1 eq) and TEA (6.13 mg, 60.61 μmol, 8.44 μL, 1 eq) in DCM (1 mL) at 0° C. The mixture was stirred at 0° C. for 1 h to give a yellow solution. LCMS indicated the product MS value was observed. The mixture was quenched by NH₄Cl (20 mL), the organic layer was washed by HCl (20 mL, pH=3˜4), and NaHCO₃ (saturated, aq., 20 mL). The organic layer was dried over anhydrous sodium sulfate and concentrated under reduced pressure to afford the crude product. The crude product was purified by prep-TLC (petroleum ether:EtOAc=3:1) to afford 09-11 (14.6 mg, 25.05 μmol, 41.33% yield).

### General Procedure for the Preparation of Compound B-72

To a solution of 09-11 (21.5 mg, 36.89 μmol, 1 eq) in THF (3 mL) was added TBAF (1 M, 44.26 μL, 1.2 eq) at −78° C. The mixture was stirred at −78° C. for 30 min to give a yellow solution. LCMS indicated the product MS value was observed. The mixture was diluted with H₂O (10 mL). The resulting mixture was extracted by EtOAc (10 mL*2). The combined organic layers were dried over anhydrous sodium sulfate and concentrated under reduced pressure to afford the crude product. The crude product was purified by flash column on silica gel eluting with 20% EtOAc in petroleum ether to afford product which was dried by lyophilization to afford B-72 (12.77 mg, 25.00 μmol, 67.79% yield). LC-MS (m/z): 511.1 [M+H]⁺. ¹H NMR (400 MHz, CHLOROFORM-d) δ ppm 0.76-0.85 (m, 6H) 1.12-1.25 (m, 4H) 1.42-1.48 (m, 1H) 1.59 (br dd, J=7.15, 3.89 Hz, 6H) 1.71-1.85 (m, 6H) 1.97-2.07 (m, 3H) 2.61-3.22 (m, 3H) 3.76-3.87 (m, 3H) 4.37-4.63 (m, 1H) 6.21-6.37 (m, 1H) 6.66-7.25 (m, 6H) 7.27-7.36 (m, 1H).

### Procedure 60: Compound B-73

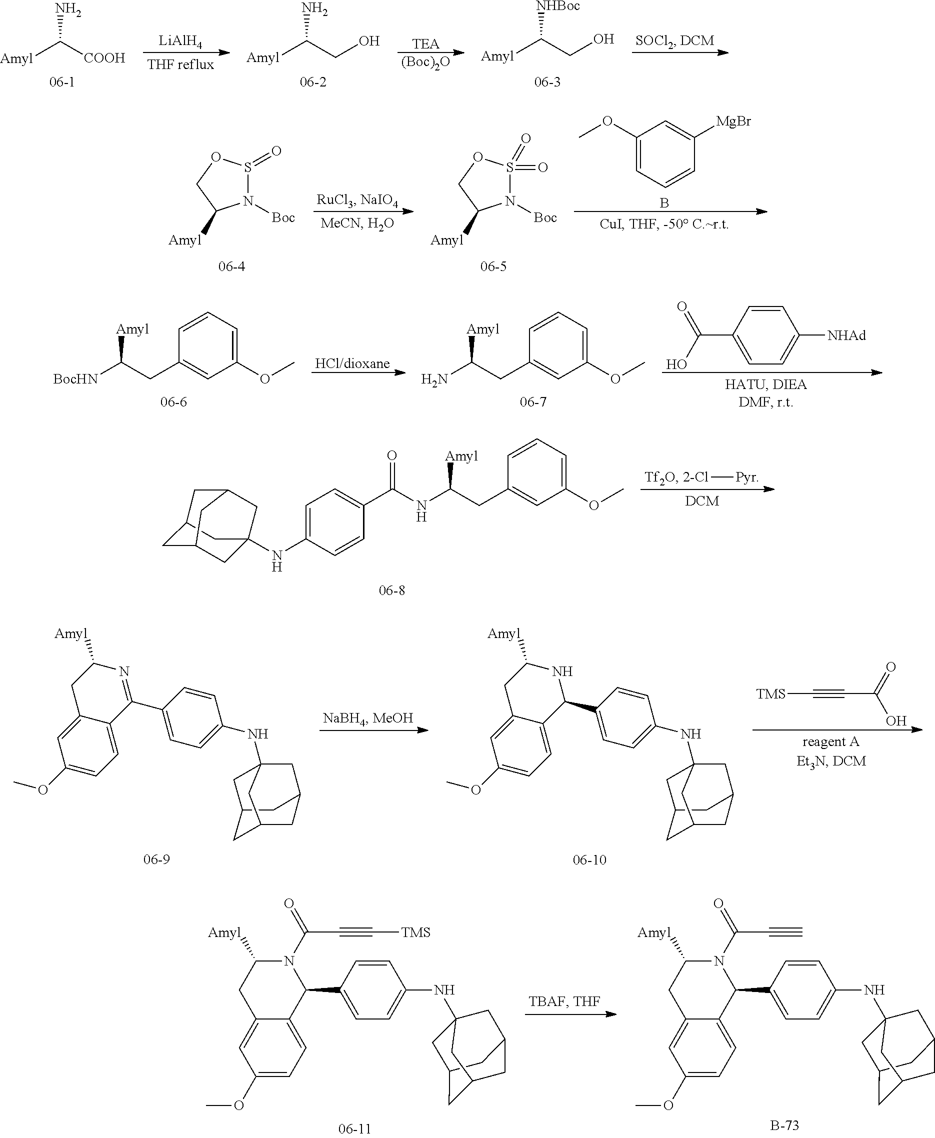

### General Procedure for the Preparation of Compound 06-2

To a solution of LAH (2.73 g, 71.90 mmol, 1.2 eq) in THF (270 mL) was added 06-1 (8.7 g, 59.92 mmol, 1 eq) at 0° C. The mixture was warmed to 25° C. and then warmed to 50° C., added THF (80 mL). The mixture was stirred at 70° C. for 12 hr to give a white suspension. The reaction was monitor by HNMR. The reaction was quenched with H₂O (2.8 mL), 15% NaOH (2.8 mL) and H₂O (8.4 mL), diluted with THF (200 mL) and dried over sodium sulfate, filtered and concentrated to give the crude product. The product 06-2 (7.1 g, 54.11 mmol, 90.31% yield) was used for next step without further purification.

### General Procedure for the Preparation of Compound 06-3

To a solution of 06-2 (7.1 g, 54.11 mmol, 1 eq) in DCM (100 mL) was added TEA (8.21 g, 81.16 mmol, 11.30 mL, 1.5 eq) and tert-butoxycarbonyl tert-butyl carbonate (10.63 g, 48.70 mmol, 11.19 mL, 0.9 eq) at 0° C. The mixture was warmed to 20° C. and stirred for 12 hr to give a clear solution. TLC (PE/EtOAc=2:1) showed the reaction was completed. The reaction was quenched with H₂O (100 mL). The separated aqueous layer was extracted with DCM (100 mL*2). The combined organic layers were washed with brine (100 mL) and dried over sodium sulfate, filtered and concentrated to give the crude product. The residue was purified by column chromatography (SiO₂, petroleum ether/ethyl acetate=1/0 to 85/15) to give 06-3 (5.92 g, 25.59 mmol, 47.29% yield).

### General Procedure for the Preparation of Compound 06-4

A solution of imidazole (10.21 g, 149.96 mmol, 5.86 eq) in DCM (120 mL) was cooled to 0° C. SOCl₂ (5.30 g, 44.53 mmol, 3.23 mL, 1.74 eq) in DCM (16 mL) was added to the solution and the result suspension was allowed to warmed to 20° C. and stirred for 1 hr at this temperature. Then the mixture was cooled to −78° C., a solution of 06-3 (5.92 g, 25.59 mmol, 1 eq) in DCM (120 mL) was added to the mixture drop wise. The result mixture was allowed to 20° C. and stirred for 12 hr to give a yellow solution. TLC (PE/EtOAc=2:1) showed the reaction was completed. The mixture was filtered and the filtrate was washed with H₂O (20 mL). The organic layer was dried over sodium sulfate and filtered to give the crude product. The product 06-4 (7.5 g, crude) was used for next step without further purification.

### General Procedure for the Preparation of Compound 06-5

RuCl₃·3H₂O (33.47 mg, 127.98 μmol, 0.005 eq) was added to a stirred mixture of 06-4 (7.1 g, 25.60 mmol, 1 eq) in MeCN (70 mL) and H₂O (50 mL) at 0° C., followed by portion wise addition of NaIO₄ (6.02 g, 28.16 mmol, 1.56 mL, 1.1 eq). The mixture was allowed to warm to 20° C. and stirred for 12 hr to give a black suspension. TLC (PE/EtOAc=4:1) showed the reaction was completed. The reaction was filtered through celite and washed with EtOAc (30 mL). The filtrate was extracted with EtOAc (30 mL*2). The combined organic layers were washed with brine (30 mL) and dried over sodium sulfate, filtered and concentrated to give the crude product. The residue was purified by column chromatography (SiO₂, Petroleum ether/Ethyl acetate=1/0 to 93/7) to give 06-5 (6.1 g, 20.79 mmol, 81.23% yield).

### General Procedure for the Preparation of 06-6

To a solution of CuI (7.79 mg, 40.90 μmol, 0.012 eq) was placed in a was added bromo-(3-methoxyphenyl)magnesium (1 M, 4.09 mL, 1.2 eq) at 0° C. under N₂. The reaction was stirred 0° C. under N₂ for 2 hr. Then 06-5 (1 g, 3.41 mmol, 1 eq) in THF (10 mL) was added dropwise at −20° C. The reaction was allowed to stir at 20° C. for 12 hr to give a yellow solution. TLC (PE/EtOAc=5:1) showed the reaction was completed. The reaction was quenched with saturated citric acid (20 mL) and extracted with EtOAc (20 mL*3). The organic layers were dried over Na₂SO₄ and concentrated to give the crude product. The residue was purified by column chromatography (SiO₂, Petroleum ether/Ethyl acetate=1/0 to 93/7) to give 06-6 (760 mg, 2.36 mmol, 69.36% yield).

### General Procedure for the Preparation of 06-7

To solution of 06-6 (760 mg, 2.36 mmol, 1 eq) was added HCl/dioxane (4 M, 10 mL, 16.92 eq), The mixture was stirred at 15° C. for 12 hr to give a yellow suspension. TLC (PE/EtOAc=5:1) showed the reaction was completed. The mixture was concentrated to give the product. The product 06-7 (630 mg, crude, HCl) which was used for next step without further purification.

### General Procedure for the Preparation of 06-8

To a solution of 4-(1-adamantylamino)benzoic acid (705.70 mg, 2.60 mmol, 1.1 eq) in DCM (7 mL) was added HATU (1.08 g, 2.84 mmol, 1.2 eq) and DIEA (916.69 mg, 7.09 mmol, 1.24 mL, 3 eq). The mixture was stirred at 20° C. for 1 hr. Then 06-7 (609.5 mg, 2.36 mmol, 1 eq, HCl) in DCM (7 mL) was added. The reaction was stirred at 20° C. for 11 hr to give a yellow solution. LCMS showed the reaction was completed. The mixture was concentrated under reduced pressure to afford the crude product. The residue was purified by column chromatography (SiO₂, Petroleum ether/Ethyl acetate=100/0 to 62/38) to give 06-8 (880 mg, 1.85 mmol, 78.41% yield).

### General Procedure for the Preparation of 06-9

To a solution of 06-8 (880 mg, 1.85 mmol, 1 eq) in DCM (12 mL) was added 2-chloropyridine (631.50 mg, 5.56 mmol, 526.25 μL, 3 eq) and Tf₂O (1.57 g, 5.56 mmol, 917.64 μL, 3 eq) at −78° C. under N₂. After 15 min, the reaction mixture was placed in an ice-water bath and warmed to 0° C. After 15 min, the resulting solution was allowed to warm to 20° C. The reaction was allowed to stir at 20° C. under N₂ for 1 hr to give a yellow solution. LCMS showed the reaction was completed. The mixture was quenched with saturated NaHCO₃ (15 mL), extracted with DCM (15 mL*3). The organic layers were dried over sodium sulfate, filtered and concentrated under vacuum. The residue was purified by column chromatography (SiO2, Petroleum ether/Ethyl acetate=100/0 to 60/40) to give 06-9 (810 mg, 1.77 mmol, 95.68% yield).

### General Procedure for the Preparation of 06-10

To a solution of 06-9 (100 mg, 218.98 μmol, 1 eq) in MeOH (3 mL) was added NaBH₄ (41.42 mg, 1.09 mmol, 5 eq) at 50° C. The reaction was stirred 1 hr to give a yellow solution. TLC (EtOAc) showed the reaction was completed. The mixture was added NaHCO₃ (30 mL) and filtered. Then extracted with EtOAc (30 mL*3). The organic layers were dried over Na₂SO₄, filtered and concentrated under vacuum. The crude product was purified by prep-TLC (SiO2, Petroleum ether/Ethyl acetate=1/1) to give 06-10 (20 mg, 43.60 μmol, 19.91% yield).

### General Procedure for the Preparation of 06-11

To a solution of 3-trimethylsilylprop-2-ynoic acid (6.20 mg, 43.60 μmol, 1 eq) in DCM (1 mL) was added 2-chloro-1-methyl-pyridin-1-ium; iodide (11.14 mg, 43.60 μmol, 1 eq) The reaction was stirred at 20° C. for 0.5 hr. Then the mixture was added dropwise in solution of 06-10 (20 mg, 43.60 μmol, 1 eq) and TEA (8.82 mg, 87.21 μmol, 12.14 μL, 2 eq) in DCM (1 mL) at 0° C. The reaction was stirred at 0° C. for 1 hr to give a yellow solution. LCMS showed the reaction was completed. The reaction mixture was washed with saturated NH₄Cl (10 mL) and extracted with DCM (10 mL*3). The organic layers were dried over Na₂SO₄ and concentrated to give the crude product. The residue was purified by prep-TLC (SiO₂, Petroleum ether/Ethyl acetate=2/1) to give 06-11 (13 mg, 22.30 μmol, 51.15% yield).

### General Procedure for the Preparation of B-73

To a solution of 06-11 (35 mg, 60.05 μmol, 1 eq) in THF (2 mL) was added TBAF (1 M, 66.05 μL, 1.1 eq) at −78° C. The reaction was stirred at −78° C. for 15 min to give a yellow solution. LCMS showed the reaction was completed. The reaction mixture was quenched H₂O (10 mL) and extracted with EtOAc (10 mL*3). The organic layers were dried over Na₂SO₄ and concentrated to give the crude product. The residue was purified by column chromatography (SiO₂, Petroleum ether/Ethyl acetate=100/0 to 74/26) to give B-73 (9.73 mg, 19.05 μmol, 31.73% yield). LC-MS (m/z): 511.5[M+H]⁺. ¹H NMR (CDCl₃, 400 MHz): δ=11.24-10.71 (m, 1H), 7.51-7.34 (m, 1H), 7.23-6.91 (m, 3H), 6.89-6.67 (m, 2H), 6.37-6.11 (m, 1H), 4.79-4.41 (m, 1H), 3.88-3.74 (m, 3H), 3.26-2.64 (m, 1H), 3.26-2.64 (m, 2H), 2.13-1.95 (m, 3H), 1.94-1.77 (m, 6H), 1.60 (br s, 2H), 1.51-1.38 (m, 4H), 1.26 (br s, 6H), 0.90-0.82 ppm (m, 3H).

### Procedure 61: Compound B-74

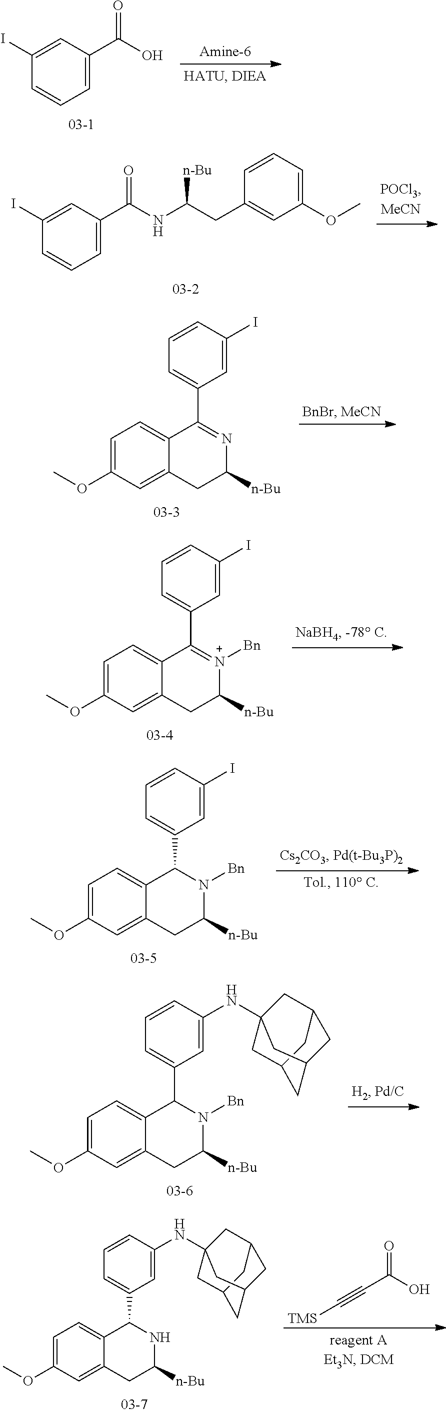

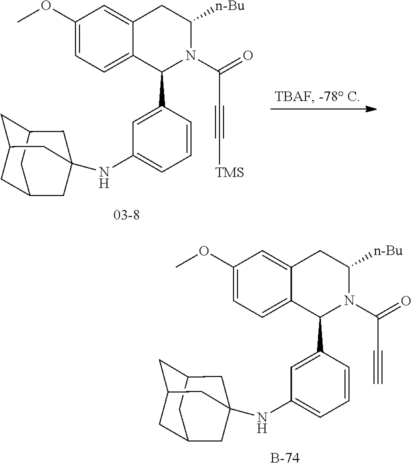

### General Procedure for the Preparation of Compound 03-2

To a solution of 03-1 (500 mg, 2.02 mmol, 1 eq) in DMF (6 mL) was added HATU (919.85 mg, 2.42 mmol, 1.2 eq), DIEA (651.37 mg, 5.04 mmol, 877.85 μL, 2.5 eq) and (2S)-1-(3-methoxyphenyl) hexan-2-amine (501.52 mg, 2.42 mmol, 1.2 eq). The reaction was stirred at 20° C. for 3 hr to give yellow suspension. LCMS showed the desired MS (as a major peak). This reaction mixture was added to MTBE (60 mL). The combined organic phase was washed with water (60 mL*3). The organic layers were dried over Na₂SO₄ and concentrated to give the crude product. The crude product was purified by silica column (PE to PE:EtOAc=5:1) to give 03-2 (680 mg, 1.55 mmol, 77.13% yield).

### General Procedure for the Preparation of Compound 03-3

To a solution of 03-2 (379.14 mg, 866.98 μmol, 1 eq) in MeCN (10 mL) was added POCl₃ (797.62 mg, 5.20 mmol, 483.41 μL, 6 eq). The reaction was stirred at 90° C. for 2 hr to give yellow suspension. LCMS showed the desired MS (as a major peak). The reaction mixture was poured into H₂O (40 mL). The combined organic layer was with NaHCO₃ (20 mL) to adjust pH=8 and extracted with EtOAc (40 mL*3). The organic layers were dried over Na₂SO₄ and concentrated to give the crude product. The residue was purified by column chromatography (SiO₂, Petroleum ether/Ethyl acetate=0% to 20%) to give 03-3 (302 mg, 720.25 μmol, 83.08% yield).

### General Procedure for the Preparation of Compound 03-4

To a solution of 03-3 (447 mg, 1.07 mmol, 1 eq) in MeCN (8 mL) was added BnBr (911.65 mg, 5.33 mmol, 633.09 μL, 5 eq). The reaction was stirred at 90° C. for 12 hr to give yellow suspension. LCMS showed the desired MS (as a major peak). The reaction mixture was concentrated to give the crude product. The crude product was triturated with PE (20 mL) to give 03-4 (650 mg, 1.04 mmol, 97.09% yield).

### General Procedure for the Preparation of Compound 03-5

To a solution of 03-4 (650 mg, 1.10 mmol, 1 eq) in MeOH (1 mL)/THF (6 mL) was added NaBH₄ (83.31 mg, 2.20 mmol, 2 eq). The reaction was stirred at −78° C. for 2 hr to give yellow suspension. LCMS showed the desired MS (as a major peak). The reaction mixture was quenched with saturated NH₄Cl (20 mL) and extracted with EtOAc (20 mL*3). The organic layers were dried over Na₂SO₄ and concentrated to give the crude product. The crude product was purified by silica column (PE to PE:EtOAc=5:1) to give 03-5 (390 mg, 762.56 μmol, 60.53% yield).

### General Procedure for the Preparation of 03-6

To a solution of 03-5 (100.00 mg, 195.53 μmol, 1 eq) in toluene (2 mL) was added adamantan-1-amine (88.72 mg, 586.58 μmol, 3 eq), Cs₂CO₃ (191.12 mg, 586.58 μmol, 3 eq) and Pd(t-Bu₃P)₂ (9.99 mg, 19.55 μmol, 0.1 eq) under N₂. The reaction was stirred at 110° C. for 12 hr to give a brown suspension. TLC (PE/EtOAc=5:1) showed the mixture was completed. The reaction mixture was quenched with H₂O (5 mL) and extracted with EtOAc (10 mL*2). The organic layers were dried over sodium sulfate, filtered and concentrated under vacuum. The residue was purified by prep-TLC (SiO₂, Petroleum ether/Ethyl acetate=5/1) to give 03-6 (40 mg, 74.80 μmol, 38.25% yield).

### General Procedure for the Preparation of 03-7

To a solution of 03-6 (150 mg, 280.49 μmol, 1 eq) in MeOH (40 mL) was added Pd(OH)₂ (31.78 mg, 45.26 μmol, 1.61 e-1 eq) under N₂. The suspension was degassed under vacuum and purged with H₂ several times. The mixture was stirred under H₂ (566.60 ug, 280.49 μmol, 1 eq) 15 PSI at 20° C. for 16 hr to give a black suspension. LCMS showed the reaction was completed. The reaction mixture was filtered on celite and washed with MeOH (50 mL). The filtrate was concentrated, then washed with saturated Na₂CO₃ (30 mL) and extracted with EtOAc (40 mL*2). The organic layers were dried over Na₂SO₄ and concentrated to give the crude product. The residue was purified by prep-TLC (SiO₂, Ethyl acetate), column chromatography (SiO₂, petroleum ether/ethyl acetate=100/0 to 62/38) and prep-TLC (SiO₂, petroleum ether/ethyl acetate=1/1) to give 03-7 (63 mg, 141.68 μmol, 50.51% yield).

### General Procedure for the Preparation of 03-8

To a solution of 3-trimethylsilylprop-2-ynoic acid (9.60 mg, 67.47 μmol, 1 eq) in DCM (1 mL) was added 2-chloro-1-methyl-pyridin-1-ium; iodide (17.24 mg, 67.47 μmol, 1 eq). The reaction was stirred at 20° C. for 0.5 hr. Then the mixture was added dropwise in solution of 03-7 (30 mg, 67.47 μmol, 1 eq) and TEA (13.65 mg, 134.94 μmol, 18.78 μL, 2 eq) in DCM (1 mL) at 0° C. The reaction was stirred at 0° C. for 1 hr to give a yellow solution. LCMS showed the reaction was completed. The reaction mixture was washed with saturated NH₄Cl (10 mL) and extracted with DCM (10 mL*3). The organic layers were dried over Na₂SO₄ and concentrated to give the crude product. The residue was purified by prep-TLC (SiO₂, petroleum ether/ethyl acetate=2/1) to give 03-8 (25 mg, 43.95 μmol, 65.14% yield).

### General Procedure for the Preparation of B-74

To a solution of 03-8 (20 mg, 35.16 μmol, 1 eq) in THF (2 mL) was added TBAF (1 M, 38.67 μL, 1.1 eq) at −78° C. The reaction was stirred at −78° C. for 15 min to give a yellow solution. LCMS showed the reaction was completed. The reaction mixture was quenched H₂O (10 mL) and extracted with EtOAc (10 mL*3). The organic layers were dried over Na₂SO₄ and concentrated to give the crude product. The residue was purified by column chromatography (SiO₂, Petroleum ether/Ethyl acetate=100/0 to 74/26) to give B-74 (14.66 mg, 29.52 μmol, 83.95% yield). LC-MS (m/z): 497.5[M+H]⁺. ¹H NMR (CDCl₃, 400 MHz): δ=7.39-7.28 (m, 1H), 7.24-7.12 (m, 1H), 7.04-6.61 (m, 4H), 6.34-6.07 (m, 1H), 4.99-4.50 (m, 1H), 3.87-3.73 (m, 3H), 3.57-2.66 (m, 3H), 2.09-1.73 (m, 9H), 1.56 (br s, 6H), 1.34-1.18 (m, 6H), 0.84 ppm (br d, J=6.8 Hz, 3H).

### Procedure 62: Compound B-82

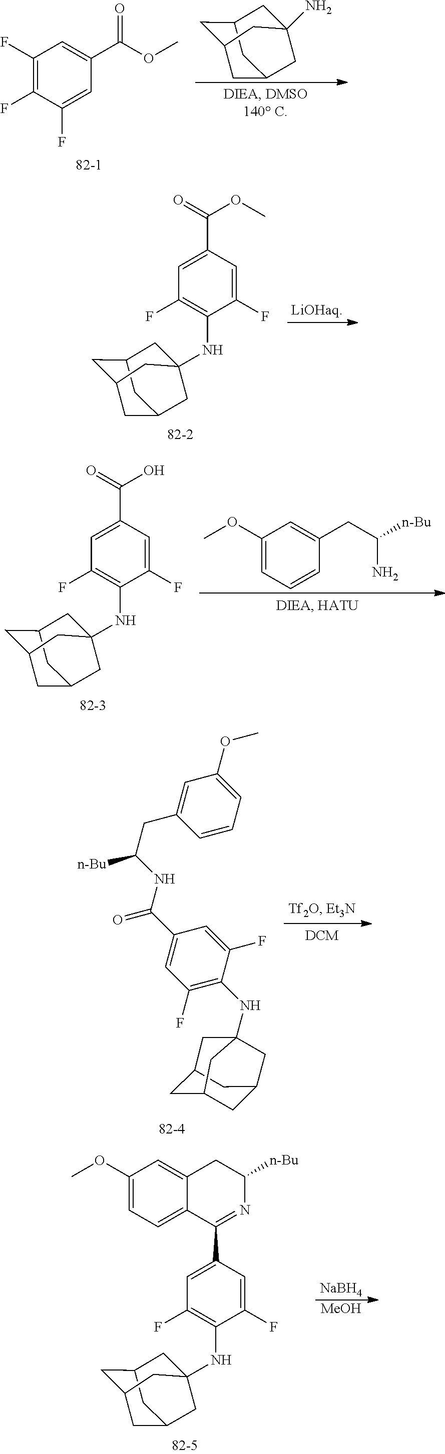

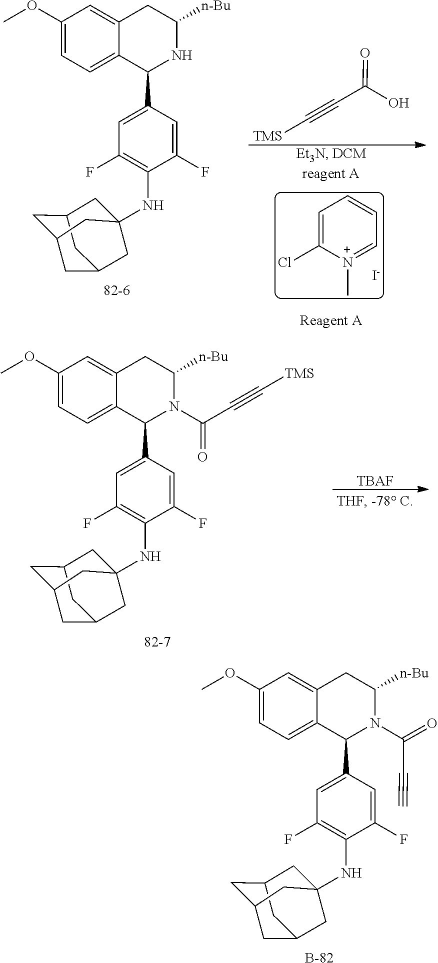

### General Procedure for the Preparation of Compound 82-2

To a solution of 82-1 (5 g, 26.30 mmol, 1 eq) in DMSO (50 mL) were added adamantan-1-amine (7.96 g, 52.60 mmol, 2 eq) and DIEA (10.20 g, 78.90 mmol, 13.74 mL, 3 eq). The reaction was stirred at 130° C. for 12 hr to give black liquid. LCMS showed the reaction was completed. The reaction mixture was diluted with water (300 mL) and extracted with EtOAc (50 mL*3). The organic layers were dried over Na₂SO₄ and concentrated to give the crude product. The crude product was purified by flash column (SiO₂, eluting with: PE/EtOAc=0% to 20%) to give 82-2 (4.75 g, 14.78 mmol, 56.20% yield).

### General Procedure for the Preparation of Compound 82-3

To a solution of 82-2 (4.75 g, 14.78 mmol, 1 eq) in EtOH (50 mL) was added LiOH·H₂O (6 M, 49.27 mL, 20 eq). The reaction was stirred at 90° C. for 12 hr to give yellow suspension. LCMS and TLC (eluting with PE/EtOAc=1/1) showed the reaction was completed. The reaction mixture was acidified with 4N HCl(aq). to pH=5, aqueous phase extracted with EtOAc (100 ml*3). The organic layers were dried over Na₂SO₄ and concentrated to give the residue. The residue was purified by flash column (SiO₂, eluting with: PE/EtOAc=0% to 50%) to give 82-3 (3.3 g, 10.74 mmol, 72.64% yield).

### General Procedure for the Preparation of Compound 82-4

To a solution of 82-3 (2 g, 6.51 mmol, 1 eq) in DMF (40 mL) was added DIEA (1.68 g, 13.02 mmol, 2.27 mL, 2 eq), (2S)-1-(3-methoxyphenyl)hexan-2-amine (1.35 g, 6.51 mmol, 1 eq) and HATU (2.60 g, 6.83 mmol, 1.05 eq) at 0° C. The reaction was allowed to stir at 0 to 25° C. for 12 hr to black brown liquid. LCMS showed the reaction was completed. The reaction mixture was quenched with H₂O (200 mL) and extracted with MTBE (60 mL*3). The organic layers were dried over Na₂SO₄ concentrated to give the crude product. The crude product was purified by flash column (eluting with PE/EtOAc=0% to 30%) to give 82-4 (3.2 g, 6.44 mmol, 99.01% yield).

### General Procedure for the Preparation of Compound 82-5

To a solution of 82-4 (3.2 g, 6.44 mmol, 1 eq) in toluene was added POCl₃ (9.88 g, 64.43 mmol, 5.99 mL, 10 eq). The reaction was stirred at 140° C. for 1 hr to give yellow mixture. LCMS showed the reaction was completed. The reaction mixture was poured into H₂O (120 ml). The mixture was basified with 1N NaOH aq. to pH=8, extracted with EtOAc (60 mL×3). The organic layers were dried over Na₂SO₄ and concentrated to give the crude product. The crude product was purified by flash column (eluting with PE/EtOAc=0% to 30%) to give 82-5 (1.8 g, 3.76 mmol, 58.37% yield).

### General Procedure for the Preparation of Compound 82-6

To a solution of 82-5 (1.7 g, 3.55 mmol, 1 eq) in MeOH (8 mL) was added NaBH₄ (671.89 mg, 17.76 mmol, 5 eq). The reaction was stirred at 25° C. for 0.5 hr to give a yellow solution. TLC (PE:EtOAc=3:1) showed the reaction was completed. The reaction mixture was quenched with saturated NH₄Cl (50 mL) and extracted with EtOAc (30 mL*3). The organic layers were dried over Na₂SO₄ and concentrated to give the crude product. The crude product was purified by flash column (eluting with PE/EtOAc=0% to 40%) to give 82a-6 (1.17 g, 2.43 mmol, 68.53% yield) and 82-6 (150 mg, 312.09 μmol, 8.79% yield).

### General Procedure for the Preparation of Compound 82-7

To a solution of 3-trimethylsilylprop-2-ynoic acid (44.39 mg, 312.09 μmol, 1 eq) in DCM (20 mL) was added 2-chloro-1-methyl-pyridin-1-ium; iodide (79.73 mg, 312.09 μmol, 1 eq). The reaction was stirred at 25° C. for 0.5 hr. Then the mixture was added dropwise the solution of 82-6 (150 mg, 312.09 μmol, 1 eq) and Et₃N (31.58 mg, 312.09 μmol, 43.44 μL, 1 eq) in DCM (20 mL) at 0° C. for 1 hr to give a yellow solution. LCMS showed the desired MS (as a major peak). The reaction mixture was quenched with H₂O (20 mL) and extracted with DCM (20 mL*3). The organic layers were washed with saturated NaHCO₃ (15 mL). The organic layers were dried over Na₂SO₄ and concentrated to give 82-7 (200 mg, crude).

### General Procedure for the Preparation of Compound 82a-7

To a solution of 3-trimethylsilylprop-2-ynoic acid (13.85 mg, 97.37 μmol, 0.9 eq) in DCM (5 mL) was added 2-chloro-1-methyl-pyridin-1-ium; iodide (24.88 mg, 97.37 μmol, 0.9 eq). The reaction was stirred at 0° C. for 0.5 hr. Then the mixture was added dropwise the solution of 82a-6 (52 mg, 108.19 μmol, 1 eq) and Et₃N (10.95 mg, 108.19 μmol, 15.06 μL, 1 eq) in DCM (5 mL) at 0 to 25° C. for 1 hr. LCMS showed the desired MS (as a major peak). The reaction mixture was quenched with H₂O (20 mL) and extracted with DCM (20 mL*3). The organic layers were washed with saturated NaHCO₃ (15 mL). The organic layers were dried over Na₂SO₄ and concentrated to give 82a-7 (70 mg, crude).

### General Procedure for the Preparation of Compound B-82

To a solution of 82-7 (188 mg, 310.82 μmol, 1 eq) in THF (20 mL) was added TBAF (81.27 mg, 310.82 μmol, 1 eq). The reaction was stirred at −78° C. for 0.5 hr to give a yellow solution. LCMS showed the reaction was completed. The reaction mixture was quenched with H₂O (50 mL) and extracted with EtOAc (30 mL*3). The organic layers were dried over Na₂SO₄ and concentrated to give the crude product. The crude product was purified by flash column (eluting with PE/EtOAc=0% to 50%) to give B-82 (57 mg, 107.01 μmol, 34.43% yield). LC-MS (m/z): 533.4[M+H]⁺. ¹H NMR (400 MHz, CDCl₃) δ ppm 7.26-7.21 (m, 1H), 6.82-6.72 (m, 1H), 6.71-6.51 (m, 3H), 6.23-6.00 (m, 1H), 4.66-4.37 (m, 1H), 3.86-3.69 (m, 3H), 3.17-2.58 (m, 3H), 2.11-1.90 (m, 3H), 1.68-1.61 (m, 6H), 1.57-1.50 (m, 6H), 1.33-1.06 (m, 6H), 0.84-0.67 (m, 3H).

### Procedure 63: Compound B-83

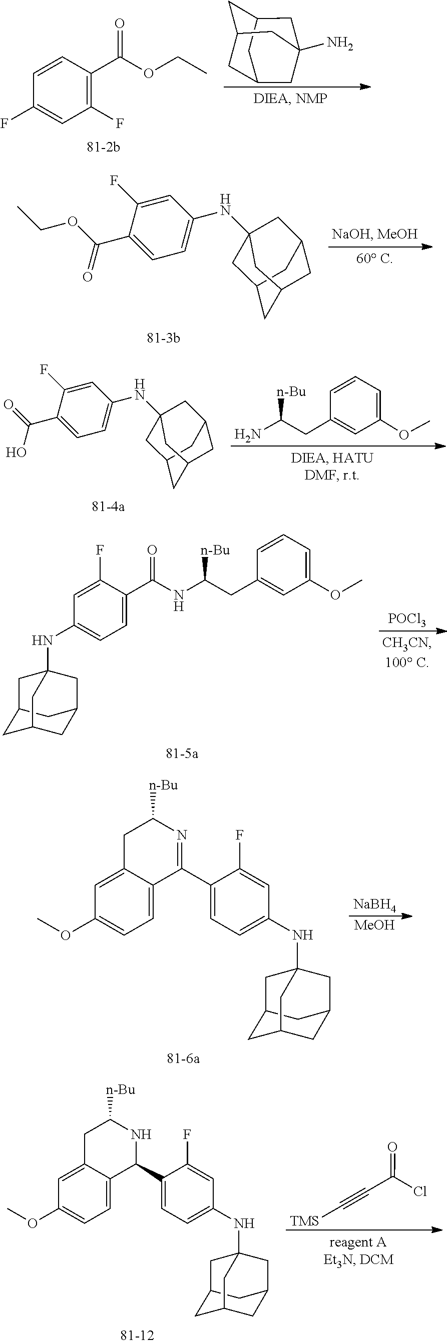

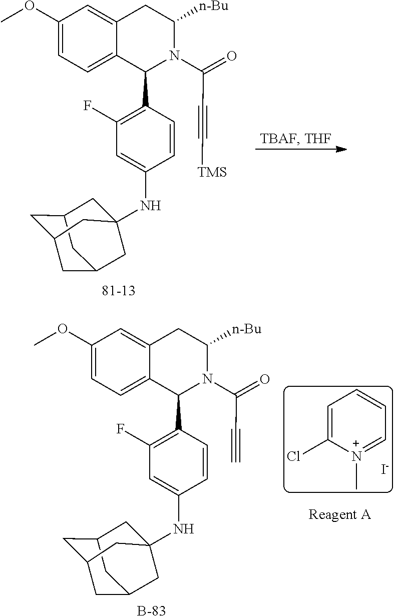

### General Procedure for the Preparation of Compound 81-3b

To a solution of adamantan-1-amine (2.11 g, 13.96 mmol, 1.3 eq.) in NMP (30 mL) were added DIEA (2.08 g, 16.11 mmol, 1.5 eq) and 81-2b (2 g, 10.74 mmol, 1 eq.). The reaction was stirred at 140° C. for 12 hr to give black solution. LCMS and TLC (PE/EtOAc=4/1) showed the reaction was completed. The reaction mixture was washed with H₂O (20 mL) and extracted with EtOAc (20 mL*3). Then the organic layers extracted with H₂O (100 mL*3). The organic layers were dried over Na₂SO₄ and concentrated to give the crude product. The residue was purified by column chromatography (SiO₂, PE/EtOAc=0% to 4%) to give 81-3b (2.6 g, 76.27% yield).

### General Procedure for the Preparation of Compound 81-4a

To a solution of 81-3b (2.6 g, 8.19 mmol, 1 eq.) in THF (30 mL)/EtOH (2 mL)/H₂O (2 mL) was added a solution of NaOH (393.17 mg, 9.83 mmol, 1.2 eq.). The reaction was stirred at 60° C. for 12 hr to give white solution. LCMS and TLC (PE/EtOAc=5/1) showed the reaction was completed. The reaction was diluted with H₂O (10 mL) and extracted with MBTE (10 mL*3). The water layer was acidified to pH=5˜6 and extracted with EtOAc (10 mL*3). The organic layers were dried over Na₂SO₄ and concentrated in vacuum to give 81-4a (1.3 g, crude).

### General Procedure for the Preparation of Compound 81-5a

To a solution of Amine 6 (569.61 mg, 2.34 mmol, 1.28 eq., HCl) in THF (10 mL) were added DIEA (591.85 mg, 4.58 mmol, 2.5 eq.) and 81-4a (530 mg, 1.83 mmol, 1 eq.) and HATU (731.30 mg, 1.92 mmol, 1.05 eq.) at 0° C. The reaction was allowed to stir at 25° C. for 12 hr to give a yellow solution. LCMS and TLC (PE/EtOAc=4/1) showed the reaction was completed. The reaction mixture was quenched with H₂O (10 mL) and extracted with MBTE (10 mL*3). Then the organic layers dried over Na₂SO₄ concentrated to dryness. The residue was purified by column chromatography (SiO₂, PE/EtOAc=0% to 16%) to give 81-5a (500 mg, 57.03% yield).

### General Procedure for the Preparation of Compound 81-6a

To a solution of 81-5a (30 mg, 62.68 μmol, 1 eq.) in CH₃CN (3 mL) was added POCl₃ (96.10 mg, 626.78 μmol, 58.25 μL, 10 eq.). The reaction was stirred at 100° C. for 2 hr to give a yellow solution. LCMS and TLC (PE/EtOAc=5/1) showed the reaction was completed. The reaction mixture was concentrated in vacuo, then washed with H₂O (5 mL) and extracted with EtOAc (5 mL*3). The organic layers were dried over Na₂SO₄ and concentrated to give the crude product. The residue was purified by prep-TLC (PE/EtOAc=10/1) to give 81-6a (15 mg, 51.96% yield).

### General Procedure for the Preparation of Compound 81-12

To a solution of 81-6a (430 mg, 933.51 μmol, 1 eq) in MeOH (15 mL) was added NaBH₄ (247.22 mg, 6.53 mmol, 7 eq). The reaction was stirred at 25° C. for 0.5 hr to give a yellow solution. LCMS and TLC (PE/EtOAc=5:1) showed the reaction was completed. The reaction mixture was quenched with saturated NH₄Cl (60 mL) and extracted with EtOAc (60 mL*3). The organic layers were dried over Na₂SO₄ and concentrated to give a yellow gum. The crude product was purified by prep-TLC (PE/EtOAc=5:1) to give 81-12 (35 mg, 69.60 μmol, 7.46% yield) and 81a-12 (200 mg, 415.01 μmol, 44.46% yield).

### General Procedure for the Preparation of Compound 81a-13

To a solution of 81a-12 (95 mg, 205.34 μmol, 1 eq) in DCM (3 mL) was added 2-chloro-1-methyl-pyridin-1-ium; iodide (52.46 mg, 205.34 μmol, 1 eq). The reaction was stirred at 20° C. for 0.5 hr. Then the mixture was added dropwise the solution of 3-trimethylsilylprop-2-ynoic acid (29.21 mg, 205.34 μmol, 1 eq) and Et₃N (20.78 mg, 205.34 μmol, 28.58 μL, 1 eq) in DCM (3 mL) at 0° C. The reaction was stirred at 0° C. for 1 hr to give a yellow solution. TLC (eluting with: PE/EtOAc=5/1) and LCMS showed the reaction was completed. The reaction mixture was quenched with H₂O (20 mL) and extracted with DCM (30 mL*2). The organic layers were washed with HCl (1N) to pH=7. The organic layer was separated. The organic layer was washed saturated NaHCO₃ (15 mL). The organic layer was separated. The organic layer was dried over Na₂SO₄ and concentrated to give 81a-13 (180 mg, crude).

### General Procedure for the Preparation of Compound 81-13

To a solution of 3-trimethylsilylprop-2-ynoyl chloride (12.16 mg, 75.65 μmol, 1 eq) in DCM (5 mL) was added 2-chloro-1-methyl-pyridin-1-ium; iodide (19.33 mg, 75.65 μmol, 1 eq). The reaction was stirred at 25° C. for 0.5 hr. Then the mixture was added dropwise the solution of 81-12 (35 mg, 75.65 μmol, 1 eq) and Et₃N (7.66 mg, 75.65 μmol, 10.53 μL, 1 eq) in DCM (5 mL) at 0° C. for 1 hr to give a yellow solution. LCMS showed the reaction was completed. The reaction mixture was quenched with H₂O (30 mL) and extracted with DCM (20 mL*3). The organic layers were dried over Na₂SO₄ and concentrated to give 81-13 (45 mg, crude).

### General Procedure for the Preparation of Compound 81a

To a solution of 81a-13 (100.00 mg, 170.40 μmol, 1 eq) in THF (10 mL) was added TBAF (1 M, 170.40 μL, 1 eq). The reaction was stirred at −78° C. for 0.5 hr to give a yellow solution. LCMS and TLC (PE/EtOAc=1:1) showed the reaction was completed. The reaction mixture was quenched with H₂O (20 mL) and extracted with EtOAc (20 mL*3). The organic layers were dried over Na₂SO₄ and concentrated to give the crude product. The crude product was purified by prep-TLC (PE/EtOAc=1:1) to give 81a (30.37 mg, 57.77 μmol, 33.90% yield). LC-MS (m/z): 515.5 [M+H]⁺. ¹H NMR (400 MHz, CDCl₃) δ ppm 7.01-6.93 (m, 1H), 6.76-6.66 (m, 2H), 6.65-6.45 (m, 2H), 6.32-5.87 (m, 2H), 5.09-5.00 (m, 1H), 4.53-4.40 (m, 1H), 3.80-3.69 (m, 3H), 3.16-2.58 (m, 3H), 2.14-2.04 (m, 3H), 1.94 (s, 6H), 1.70-1.60 (m, 6H), 1.48-1.47 (m, 2H), 1.12-0.99 (m, 4H), 0.77-0.65 (m, 3H).

### General Procedure for the Preparation of Compound B-83

To a solution of 81-13 (45 mg, 76.68 μmol, 1 eq) in THF (5 mL) was added TBAF (20.05 mg, 76.68 μmol, 1 eq). The reaction was stirred at −78° C. for 0.5 hr to give a yellow solution. LCMS and TLC (PE/EtOAc=1:1) showed the reaction was completed. The reaction mixture was quenched with H₂O (20 mL) and extracted with EtOAc (20 mL*3). The organic layers were dried over Na₂SO₄ and concentrated to give the crude product. The crude product was purified by prep-TLC (PE/EtOAc=1:1) to give B-83 (21.86 mg, 41.62 μmol, 54.28% yield). LC-MS (m/z): 515.7 [M+H]⁺. ¹H NMR (400 MHz, CDCl₃) δ ppm 7.00-6.79 (m, 11H), 6.74-6.38 (m, 4H), 6.31-6.00 (m, 2H), 5.00 (br s, 2H), 3.79-3.67 (m, 3H), 3.21-2.55 (m, 3H), 2.15-2.04 (m, 3H), 2.03-1.87 (m, 6H), 1.70-1.59 (m, 6H), 1.47 (s, 2H), 1.29-0.97 (m, 4H), 0.80-0.69 (in, 3H).

Compounds disclosed herein, can be or were, synthesized according to the procedures described above using the appropriate reagents and starting materials. Select data are shown in Table B-10.

TABLE B-10

| | | MS |
|---|---|---|
| No. | Structure | [M + H]⁺ |
| B-2 | 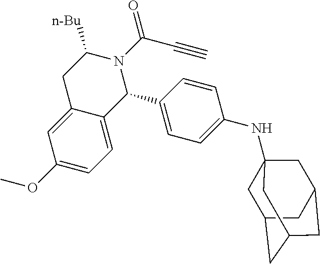 | 497.3 |
| B-5 | 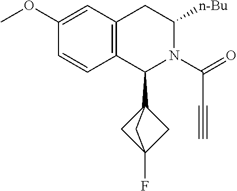 | 356.2 |
| B-6 | 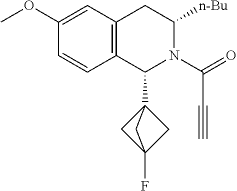 | 356.1 |
| B-12 | 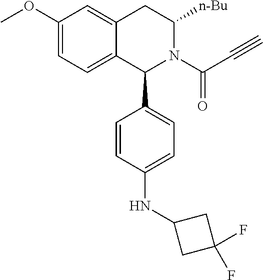 | 497.3 |
| B-13 | 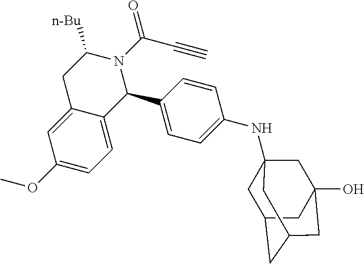 | 513.5 |
| B-14 | 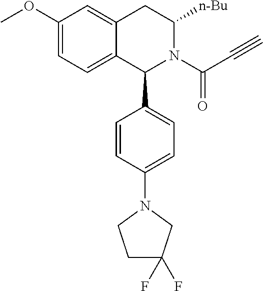 | 453.2 |
| B-19 | 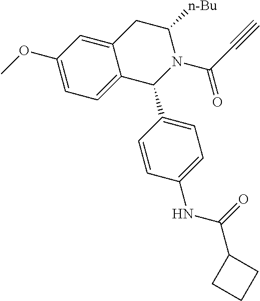 | 445.2 |
| B-20 | 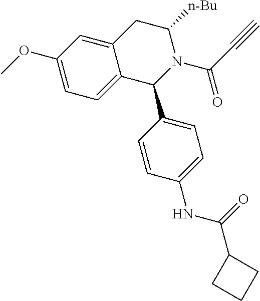 | 445.2 |
| B-21 | 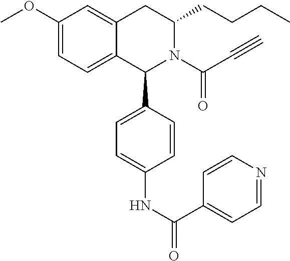 [SMILES: C#CC(=O)N1[C@@H](CCCC)Cc2cc(OC)ccc2[C@@H]1c1ccc(NC(=O)c2ccncc2)cc1] | 468.5 |
| B-22 | 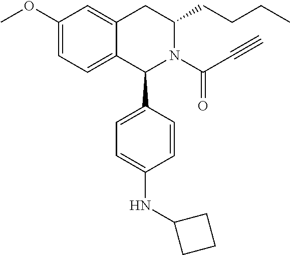 [SMILES: C#CC(=O)N1[C@@H](CCCC)Cc2cc(OC)ccc2[C@@H]1c1ccc(NC2CCC2)cc1] | 417.3 |
| B-24 | 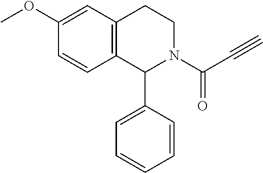 [SMILES: C#CC(=O)N1CCc2cc(OC)ccc2C1c1ccccc1] | 292.1 |
| B-26 | 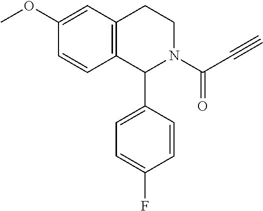 [SMILES: C#CC(=O)N1CCc2cc(OC)ccc2C1c1ccc(F)cc1] | 310.3 |
| B-28 | 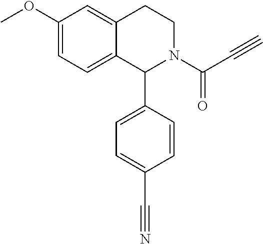 [SMILES: C#CC(=O)N1CCc2cc(OC)ccc2C1c1ccc(C#N)cc1] | 317.0 |
| B-29 | 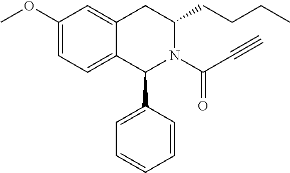 [SMILES: C#CC(=O)N1[C@@H](CCCC)Cc2cc(OC)ccc2[C@@H]1c1ccccc1] | 348.3 |
| B-31 | 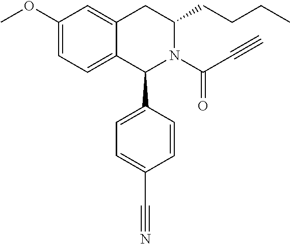 [SMILES: C#CC(=O)N1[C@@H](CCCC)Cc2cc(OC)ccc2[C@@H]1c1ccc(C#N)cc1] | 373.5 |
| B-32 | 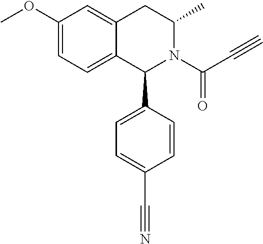 [SMILES: C#CC(=O)N1[C@@H](c2ccc(C#N)cc2)c2ccc(OC)cc2C[C@@H]1C] | 331.3 |
| B-35 | 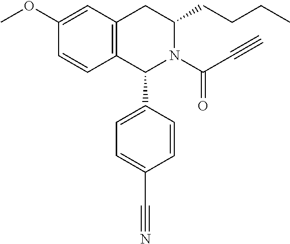 [SMILES: C#CC(=O)N1[C@@H](CCCC)Cc2cc(OC)ccc2[C@H]1c1ccc(C#N)cc1] | 365.5 |
| B-37 | 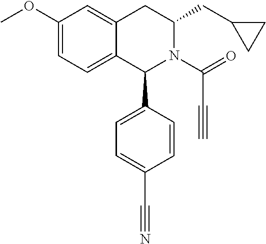 [SMILES: C#CC(=O)N1[C@@H](CC2CC2)Cc2cc(OC)ccc2[C@@H]1c1ccc(C#N)cc1] | 371.2 |
| B-39 | 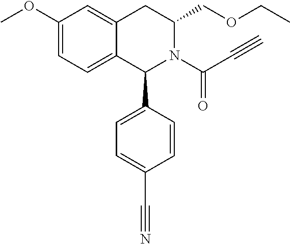 [SMILES: C#CC(=O)N1[C@@H](COCC)Cc2cc(OC)ccc2[C@@H]1c1ccc(C#N)cc1] | 375 |
| B-40 | 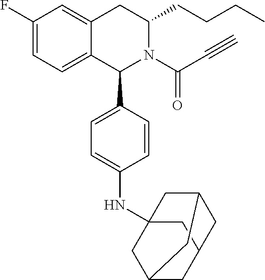 [SMILES: C#CC(=O)N1[C@@H](CCCC)Cc2cc(F)ccc2[C@@H]1c1ccc(NC23CC4CC(CC(C4)C2)C3)cc1] | 485.5 |
| B-41 | 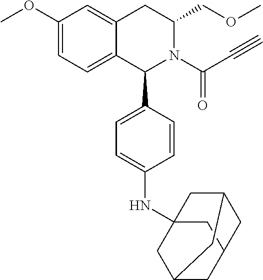 | 484.6 |
| B-42 | 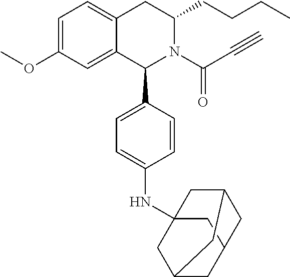 [SMILES: C#CC(=O)N1[C@@H](CCCC)Cc2ccc(OC)cc2[C@@H]1c1ccc(NC23CC4CC(CC(C4)C2)C3)cc1] | 496.7 |
| B-43 | 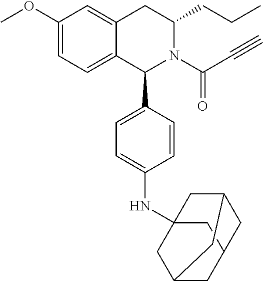 [SMILES: C#CC(=O)N1[C@@H](CCC)Cc2cc(OC)ccc2[C@@H]1c1ccc(NC23CC4CC(CC(C4)C2)C3)cc1] | 483.0 |
| B-44 | 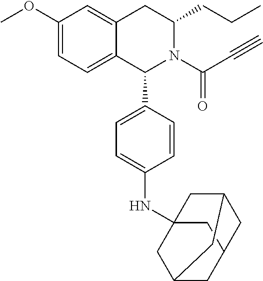 [SMILES: C#CC(=O)N1[C@@H](CCC)Cc2cc(OC)ccc2[C@H]1c1ccc(NC23CC4CC(CC(C4)C2)C3)cc1] | 483.6 |
| B-45 | 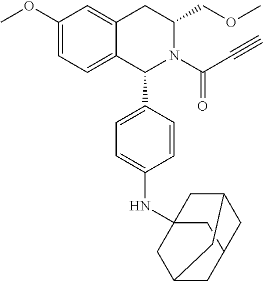 | 484.6 |
| B-46 | 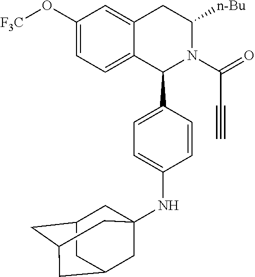 | 551.5 |
| B-50 | 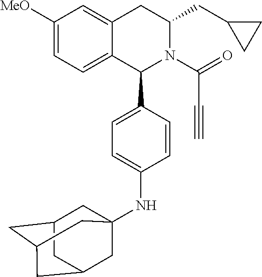 [SMILES: C#CC(=O)N1[C@@H](CC2CC2)Cc2cc(OC)ccc2[C@@H]1c1ccc(NC23CC4CC(CC(C4)C2)C3)cc1] | 495.3 |
| B-51 | 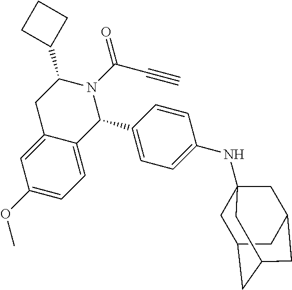 [SMILES: C#CC(=O)N1[C@H](c2ccc(NC34CC5CC(CC(C5)C3)C4)cc2)c2ccc(OC)cc2C[C@@H]1C1CCC1] | 495.3 |
| B-52 | 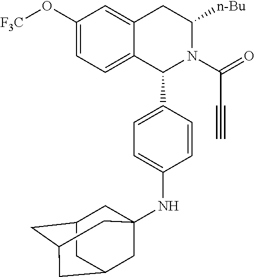 | 551.5 |
| B-53 | 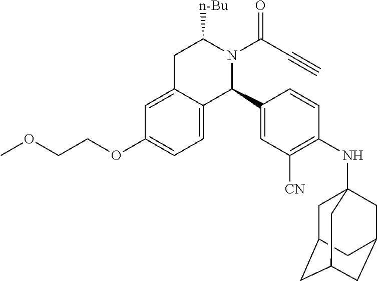 | 566.3 |
| B-54 | 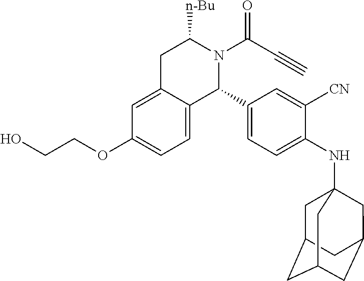 | 550.2 |
| B-55 | 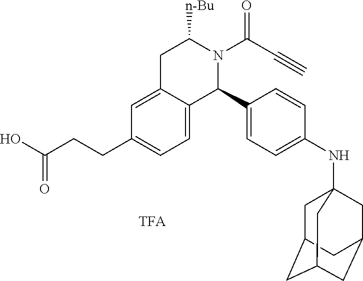 | 539.4 |
| B-56 | 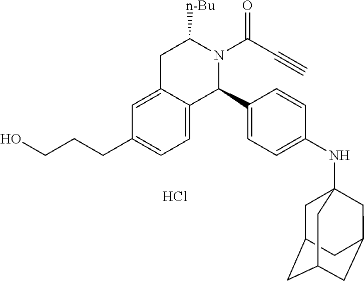 | 525.1 |
| B-57 | 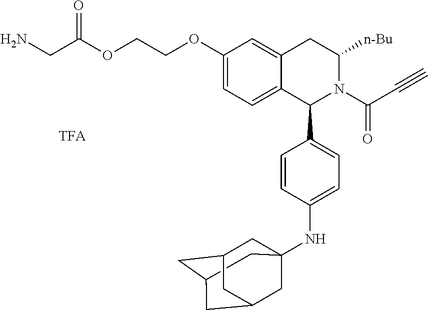 | 584.3 |
| B-58 | 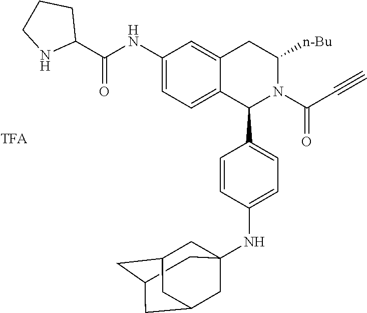 | 579.2 |
| B-59 | 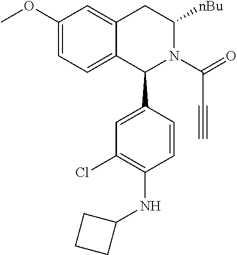 | 451.2 |
| B-60 | 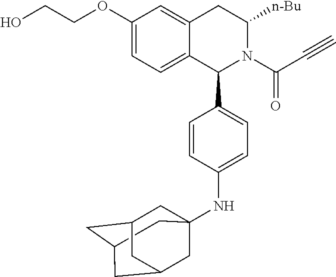 | 599.3 |
| B-61 | 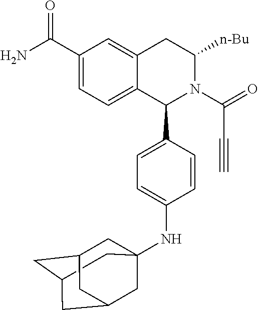 | 510.1 |
| B-62 | 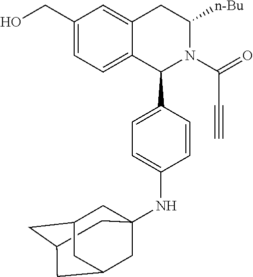 | 497.2 |
| B-63 | 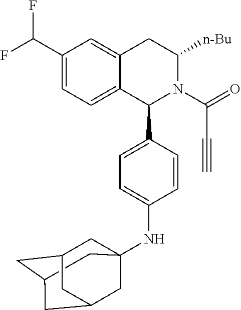 | 517.1 |
| B-64 | 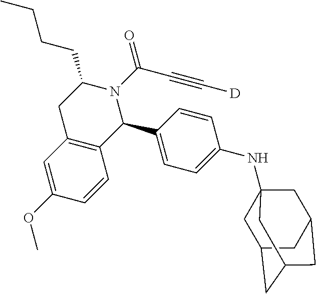 [SMILES: [2H]C#CC(=O)N1[C@@H](CCCC)Cc2cc(OC)ccc2[C@@H]1c1ccc(NC23CC4CC(CC(C4)C2)C3)cc1] | 498.1 |
| B-65 | 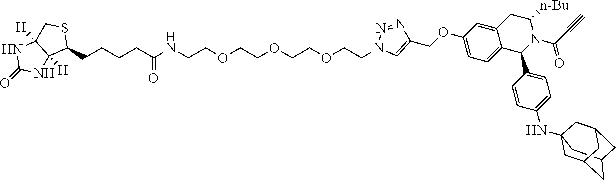 | 965.5 |
| B-66 | 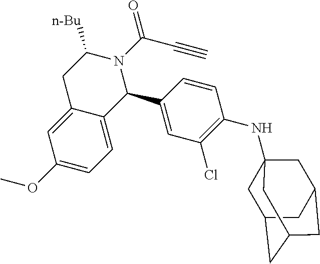 | 531.1 |
| B-67 | 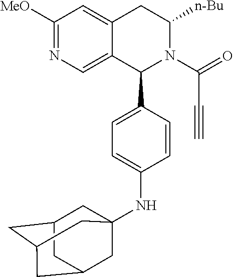 | 498.2 |
| B-68 | 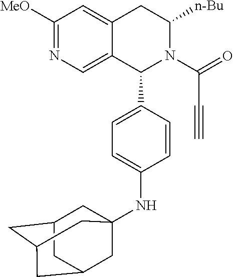 | 498.2 |
| B-69 | 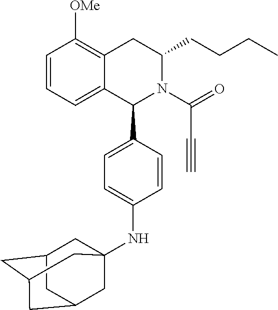 [SMILES: C#CC(=O)N1[C@@H](CCCC)Cc2c(OC)cccc2[C@@H]1c1ccc(NC23CC4CC(CC(C4)C2)C3)cc1] | 497.3 |
| B-70 | 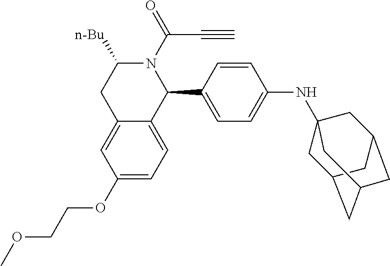 | 541.2 |
| B-71 | 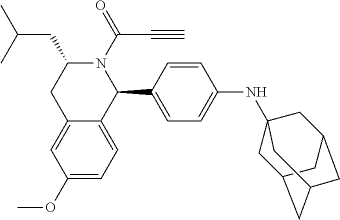 [SMILES: C#CC(=O)N1[C@@H](CC(C)C)Cc2cc(OC)ccc2[C@@H]1c1ccc(NC23CC4CC(CC(C4)C2)C3)cc1] | 497.1 |
| B-72 | 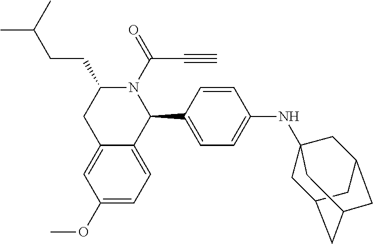 [SMILES: C#CC(=O)N1[C@@H](CCC(C)C)Cc2cc(OC)ccc2[C@@H]1c1ccc(NC23CC4CC(CC(C4)C2)C3)cc1] | 511.1 |
| B-73 | 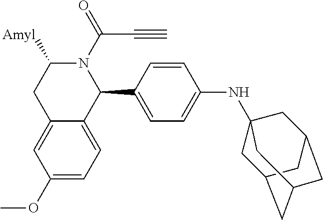 | 511.5 |
| B-74 | 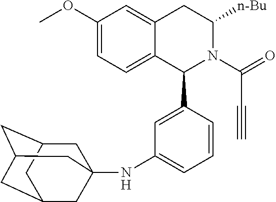 | 497.5 |
| B-75 | 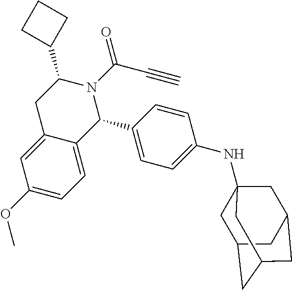 [SMILES: C#CC(=O)N1[C@H](c2ccc(NC34CC5CC(CC(C5)C3)C4)cc2)c2ccc(OC)cc2C[C@@H]1C1CCC1] | 495.3 |
| B-76 | 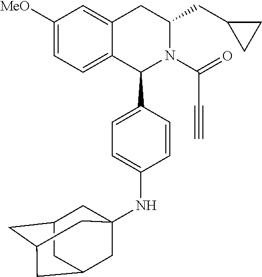 [SMILES: C#CC(=O)N1[C@@H](CC2CC2)Cc2cc(OC)ccc2[C@@H]1c1ccc(NC23CC4CC(CC(C4)C2)C3)cc1] | 495.3 |
| B-77 | 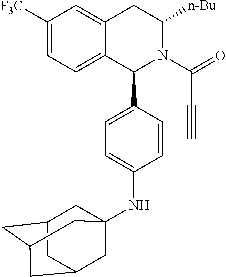 | 535.3 |
| B-78 | 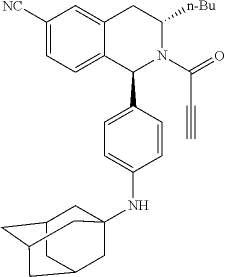 | 492.2 |
| B-79 | 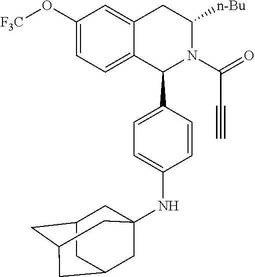 | 551.5 |
| B-80 | 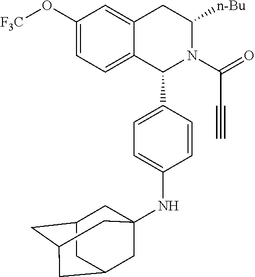 | 551.5 |
| B-81 | 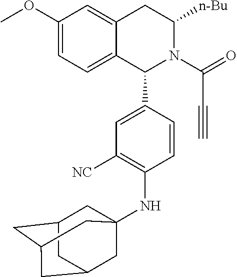 | 522.5 |
| B-82 | 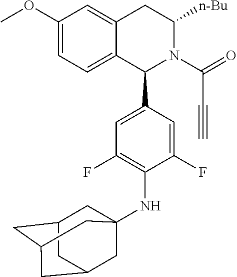 | 533.5 |
| B-83 | 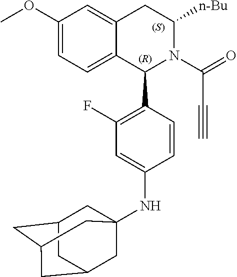 | 515.7 |
| B-84 | 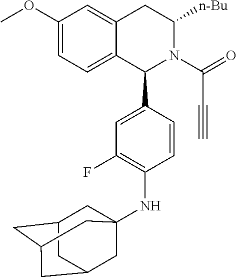 | 515.3 |

### BIOLOGICAL EXAMPLES

### Example 1: Cell Proliferation (Alamar Blue) Assay

Cell viability assay was performed to assess the potency of the compounds in human cancer cell lines 786-0 (renal cell carcinoma) and SJSA-1 (osteosarcoma). Additional cell lines, such as pancreatic cancer cell lines (Panc 02.13, BxPC-3, Panc 12, Panc 02.03, Panc 6.03, PSN-1, HPAC, and Capan-1), prostate cancer cell lines (PC-3, DU145, 22Rv0, NCI-H660, 1PH1 LNCaP, BM-1604, and MDA PCa 2b), etc., can be tested in a similar method.

Cells (SJSA-1, 786-0 and A431) were seeded (5000 cells/100 μL/well) in 96-well tissue culture plate and incubated at 37° C./500 CO₂ for 16-24 hours. The cells were then treated with compounds (25 μL of 5×). The compound concentrations were 10-0.0005 μM prepared in 3-fold serial dilutions with final DMSO concentration of 1%. The plates were then incubated for 24 h at 37° C./50% CO₂ in a moist environment. Then Alamar Blue™ reagent (final concentration 1×-12.5 μL) was added to each well and incubated for 1.5 hours at 37° C./500 CO₂. The plates were read on fluorescence reader at 540 nm excitation and 590 nm emission wavelengths. The IC₅₀ values were subsequently determined using a sigmoidal dose-response curve (variable slope) in GraphPad Prism® 5 software. Tables A-3 and B-11 show cell proliferation data for exemplary compounds as described herein.

TABLE B-11
Cell proliferation data for selected compounds of the disclosure.

| | | | | | IC₅₀ (μM) | | |
|---|---|---|---|---|---|---|---|
| Compound | IC₅₀ (μM) | | | Compound | | 786-O + | |
| No. | 786-O | SJSA-1 | A431 | No. | 786-O | Ferrastatin-1 | A431 |
| B-2 | 0.192 | | >1 | B-13 | 0.055 | 2.01 | 1.49 |
| B-4 | 0.016 | | | B-55 | 0.046 | 10 | 10 |
| B-5 | 0.145 | | >1 | B-56 | 0.012 | 1.27 | 0.117 |
| B-6 | 0.067 | | >1 | B-57 | 0.005 | 2.81 | 2.88 |
| B-8 | 0.021 | | | B-58 | 0.011 | 2.36 | 0.856 |
| B-12 | 0.065 | | >1 | B-59 | 0.052 | 5.84 | 2.37 |
| B-14 | 0.060 | | >1 | B-60 | 0.005 | 2.18 | 2.12 |
| B-19 | 0.569 | 0.520 | 6.30 | B-61 | 0.012 | 1.28 | 3.54 |
| B-20 | 0.020 | 0.021 | 6.14 | B-62 | 0.005 | 7.15 | 2.43 |
| B-22 | 0.041 | 0.050 | 9.96 | B-63 | 0.009 | 0.939 | 0.727 |
| B-24 | 0.198 | | 3.5 | B-65 | 10 | 10 | 10 |
| B-26 | 0.395 | | >10 | B-66 | 0.055 | 2.62 | 3.15 |
| B-28 | 0.18 | | 7.5 | B-67 | 0.005 | 1.19 | 1.13 |
| B-29 | 0.037 | | 8.73 | B-68 | 0.042 | 2.61 | 2.35 |
| B-31 | 0.016 | | 3.67 | B-69 | 0.015 | 2.47 | 1.79 |
| B-32 | 0.42 | | 3.50 | B-70 | 0.012 | 1.59 | 2.08 |
| B-35 | 0.1 | | 8.36 | B-71 | 0.066 | 1.71 | 1.18 |
| B-37 | 0.049 | | >10 | B-72 | 0.036 | 1.60 | 1.70 |
| B-39 | 0.195 | | >10 | B-73 | 0.044 | 1.04 | 1.46 |
| B-40 | 0.006 | | 4.52 | B-74 | 0.025 | 1.91 | 3.06 |
| B-41 | 0.0035 | | 3.88 | B-77 | 0.012 | 1.21 | 0.557 |
| B-42 | 0.0145 | | 6.58 | B-78 | 0.004 | 0.662 | 0.569 |
| B-43 | 0.006 | | 4.10 | B-79 | 0.017 | 1 | 7 |
| B-44 | 0.054 | | >1 | B-81 | 0.01 | 10 | 4.9 |
| B-45 | 0.047 | | | B-82 | 0.024 | 1.71 | 1.84 |
| B-49 | 0.037 | 0.2 | | B-83 | 2.79 | 6.59 | 7.87 |
| B-50 | 0.01 | | | B-84 | 0.037 | 0.056 | 0.881 |
| B-51 | 0.072 | | 4.90 | | | | |

### Example 2: GPX4 Inhibition Assay

Studies have shown that lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, the ability of a molecule to induce ferroptotic cancer cell death, and that such ability is admonished by the addition of ferrostatin, is clear indication that the molecule is an GPX4 inhibitor. Cell proliferation data for exemplary compounds with and without ferrostatin can be seen in Table A-3.

TABLE A-3
Cell proliferation of selected compounds.

| | IC₅₀ (μM) | | |
|---|---|---|---|
| Compound | | 786-O + | |
| No. | 786-O | Ferrastatin-1 | A431 |
| A-1 | 0.043 | 7 | >10 |
| A-2 | 0.009 | 8.63 | >10 |
| A-3 | 0.029 | >1 | |
| A-4 | 0.067 | >1 | >1 |
| A-5 | 0.039 | >1 | >1 |
| A-6 | 0.021 | >1 | >1 |
| A-7 | 0.021 | >1 | |
| A-8 | 0.064 | 5.05 | 4.79 |
| A-9 | 0.180 | >1 | >1 |
| A-10 | 0.075 | >1 | >1 |
| A-11 | 0.109 | >1 | >1 |
| A-12 | 0.051 | >1 | >1 |
| A-15 | 0.021 | >1 | |
| A-16 | 0.016 | >10 | 10 |
| A-25 | 0.171 | >1 | |
| A-26 | 0.043 | 4.19 | 3.05 |
| A-28 | 0.015 | 3.58 | 2.94 |
| A-29 | 0.014 | >1 | |
| A-33 | 0.023 | >1 | |
| A-41 | 0.0125 | 1.86 | 0.563 |
| A-43 | 0.0725 | 2.13 | 1.83 |
| A-44 | 0.0053 | 2.82 | 2.24 |
| A-45 | 0.0177 | 2.04 | 1.7 |
| A-46 | 0.310 | 10 | 10 |
| A-47 | 0.049 | 2.33 | 1.63 |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the embodiments.

What is claimed is:

1. A compound of formula A-I: 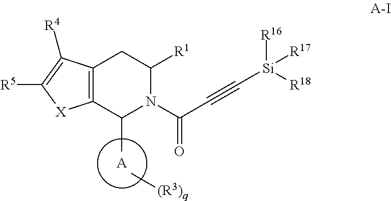
or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently further substituted with one to three R¹¹;
each R⁷ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently further substituted with one to three R¹¹;
each R⁸ is independently C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently further substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl this is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰;
provided that the compound is not:

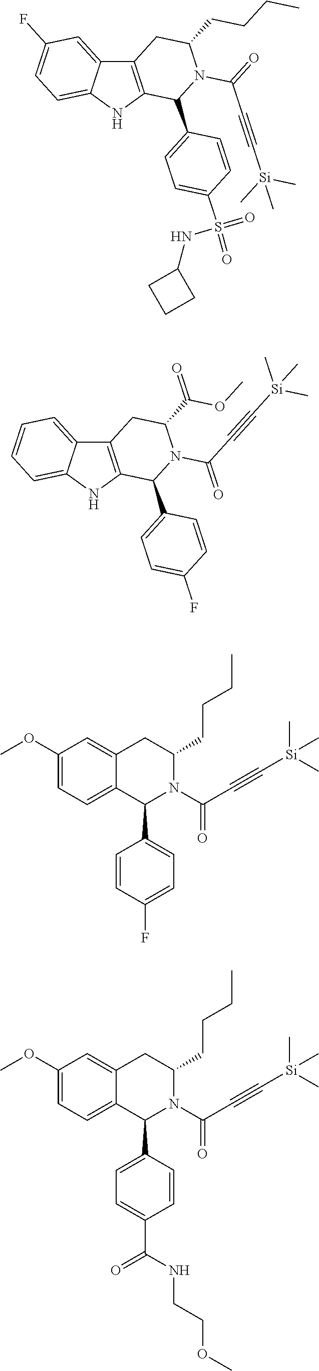

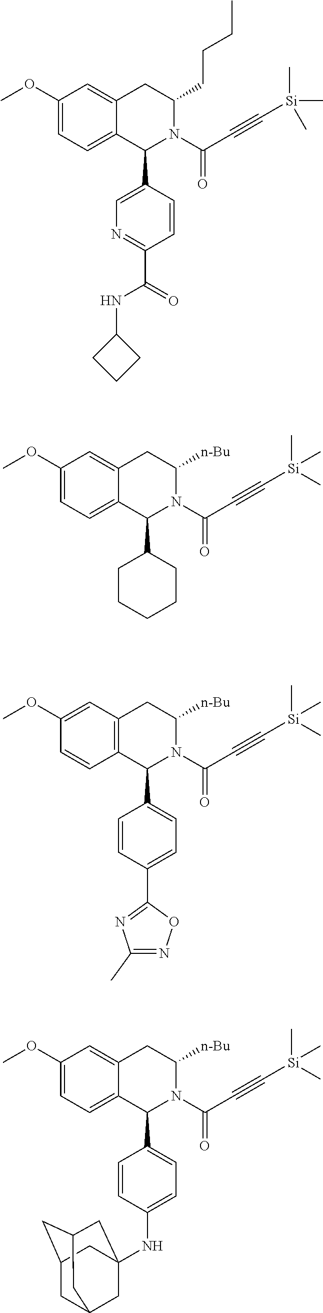

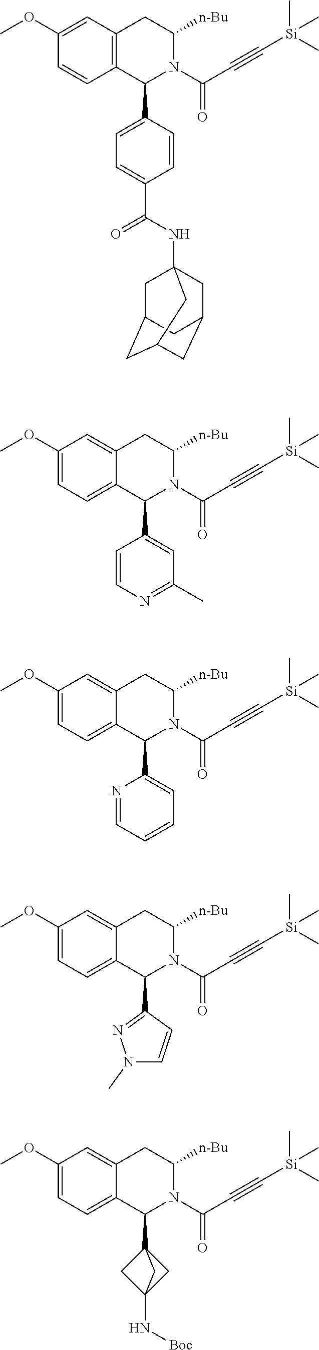

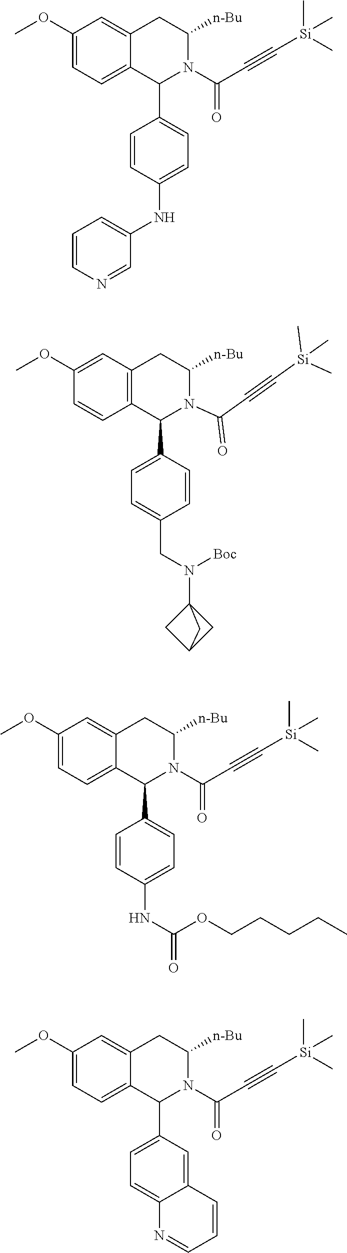

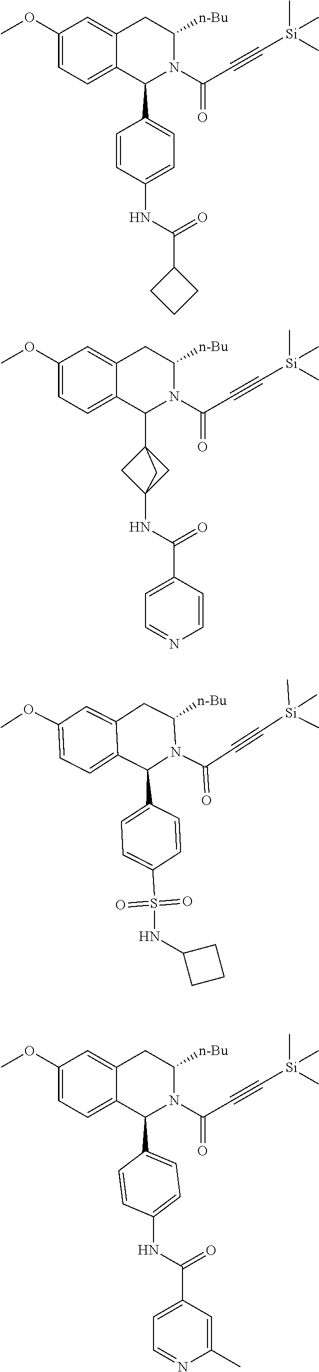

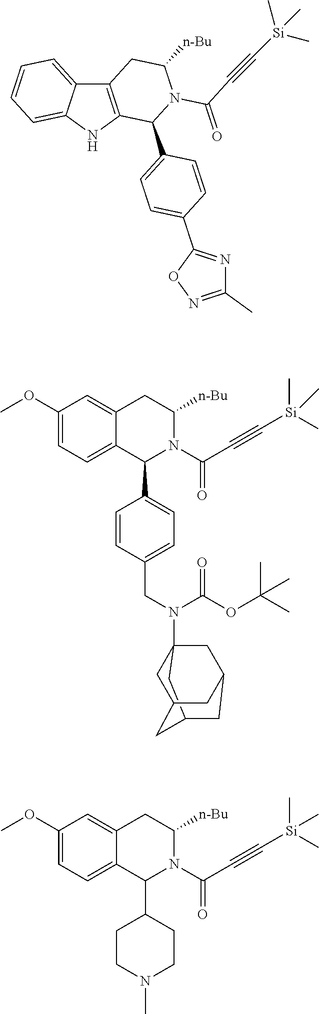

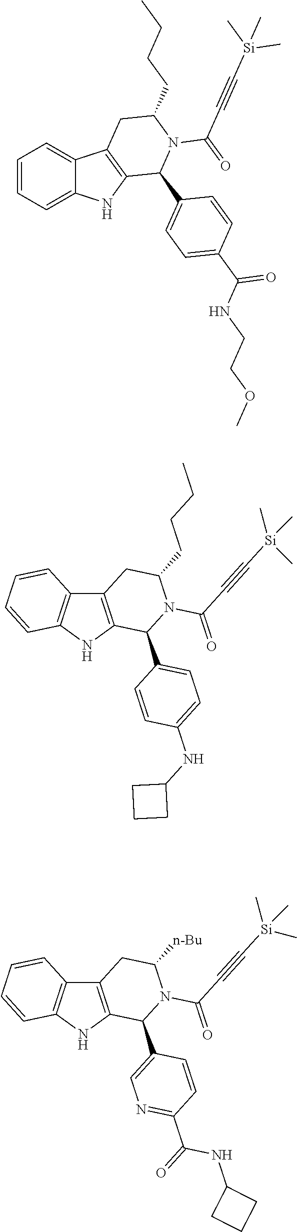

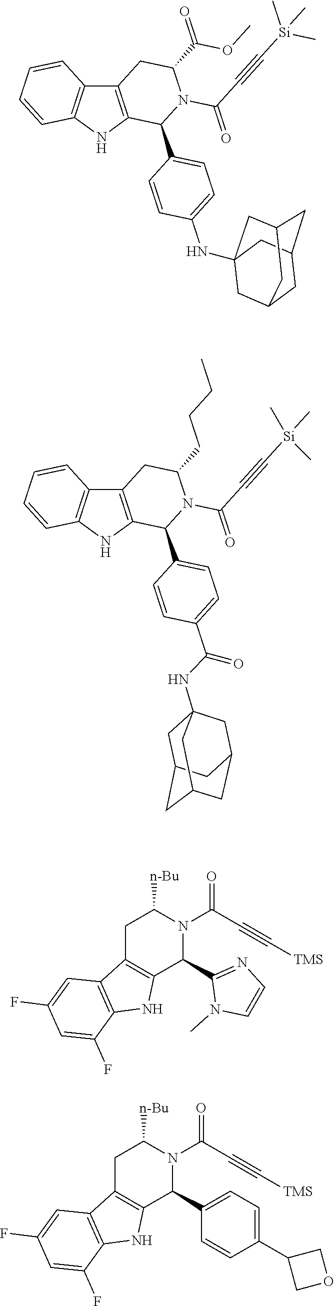

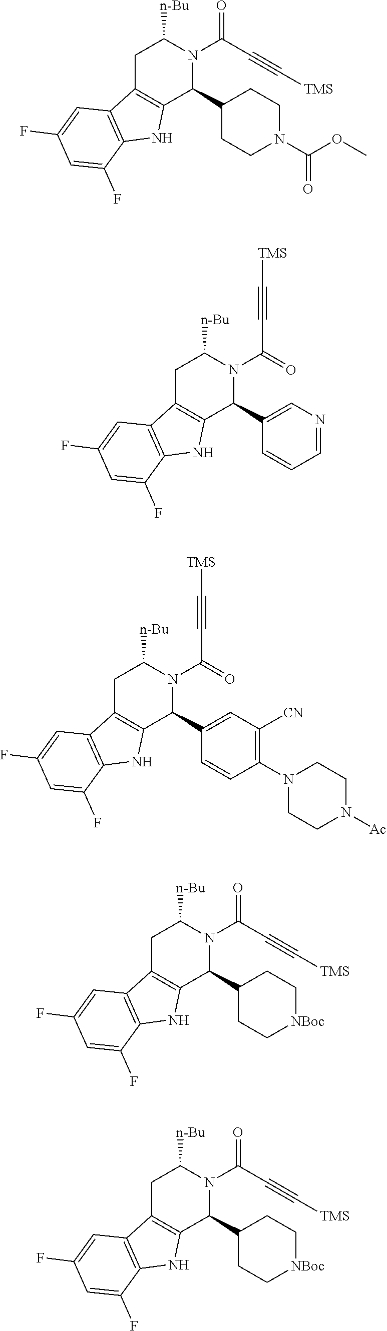

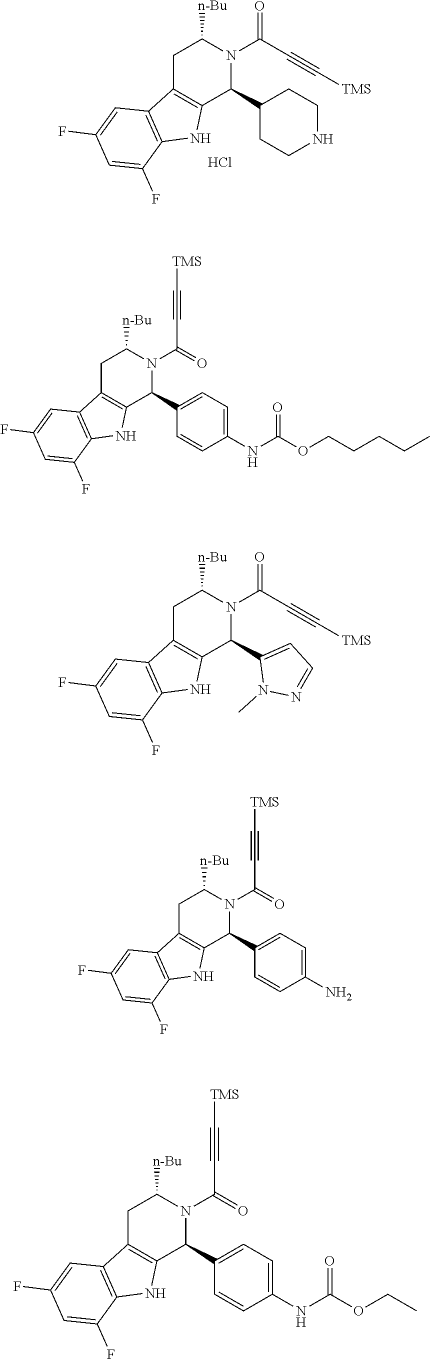

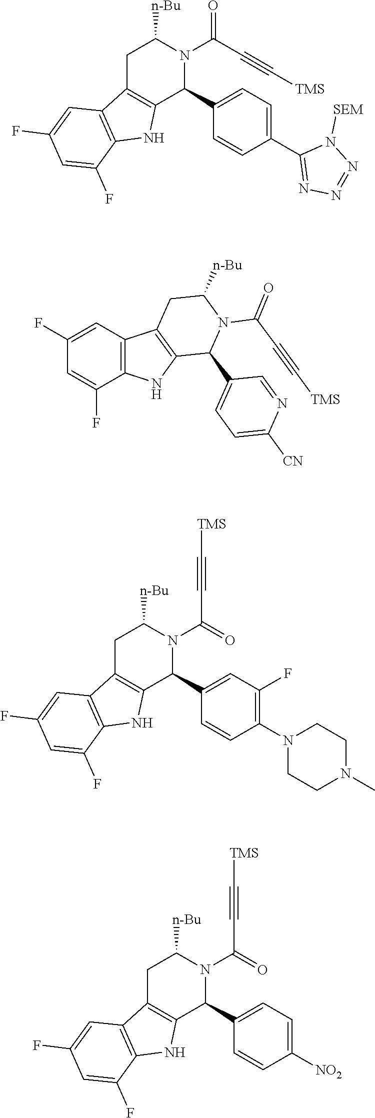

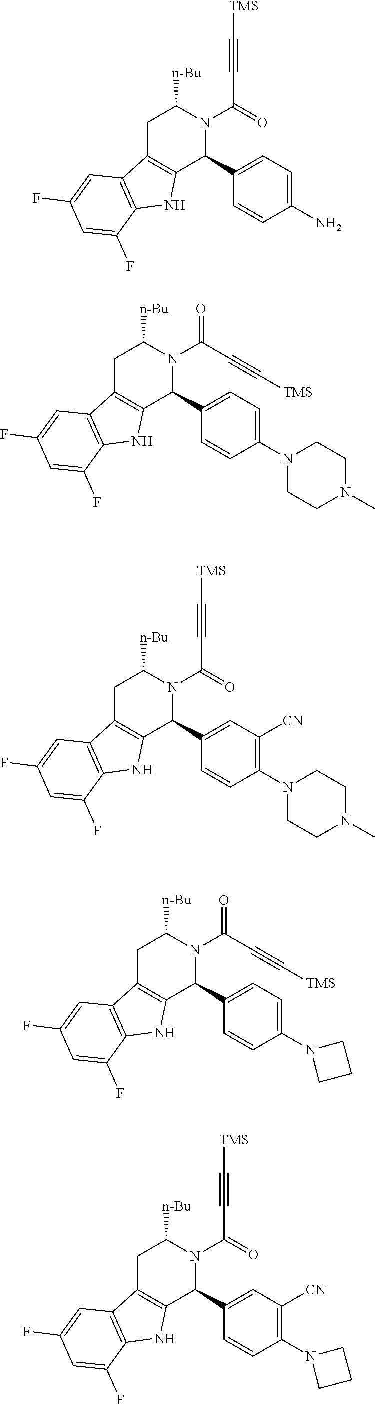

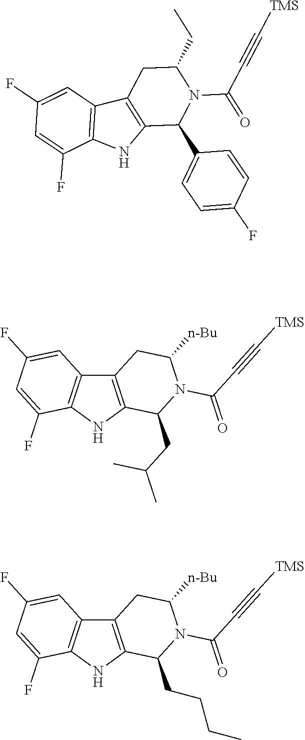

2. The compound of claim 1, or a tautomer, stereoisomer, mixture of stereoisomers, isotopically enriched analog, or pharmaceutically acceptable salt thereof, represented by formula A-IA:

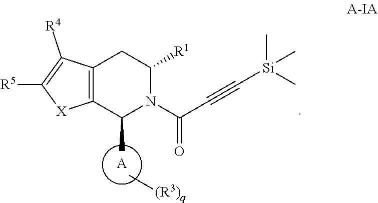

3. The compound of claim 1, or a tautomer, stereoisomer, mixture of stereoisomers, isotopically enriched analog, or pharmaceutically acceptable salt thereof, represented by formula A-IB:

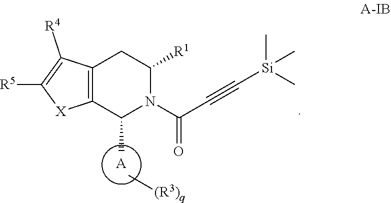

4. The compound of any one of claims 1-3, wherein X is —NR²²—, —O—, or —S—.

5. The compound of claim 4, wherein X is —NH—.

6. The compound of any one of claims 1-3, wherein X is —N═CR⁹—, —CR⁹═CR⁹—, or —CR⁹═N—.

7. The compound of claim 6, wherein X is —CR⁹═CR⁹—.

8. The compound of claim 7, wherein X is —CH═CH—.

9. A compound of formula A-I: 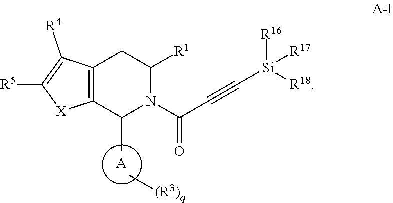
or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
each R⁶ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently further substituted with one to three R¹¹;
each R⁷ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R⁷ or ring formed thereby is independently further substituted with one to three R¹¹;
each R⁸ is independently C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently further substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰;
provided the compound is not:

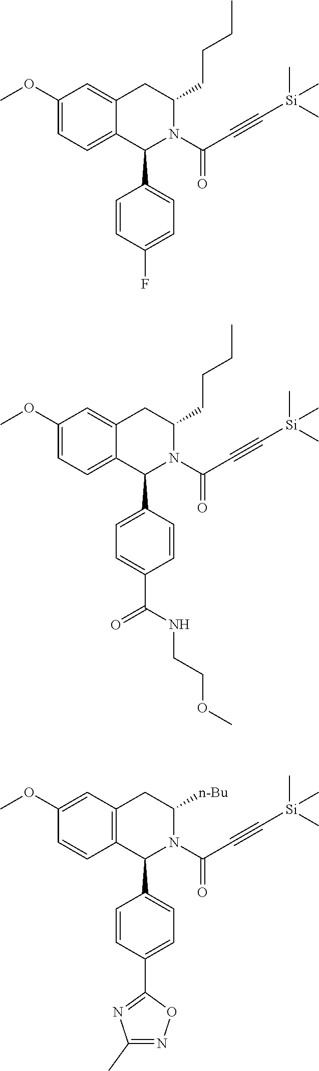

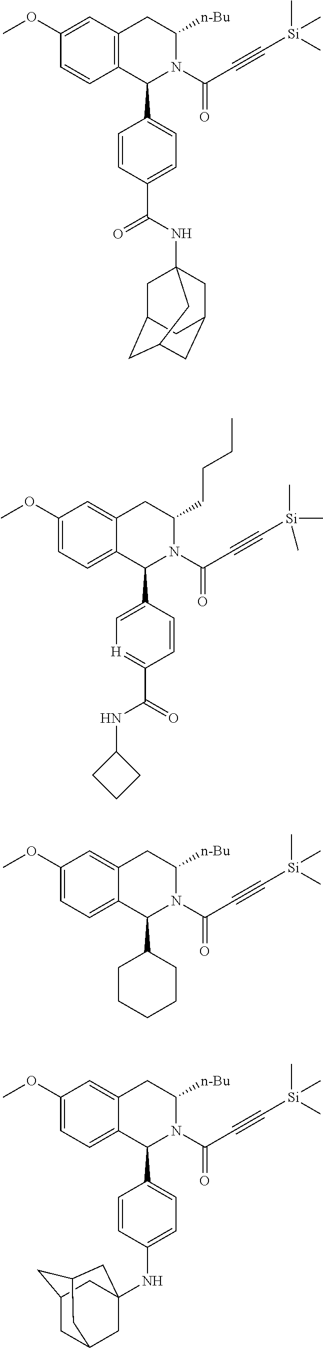

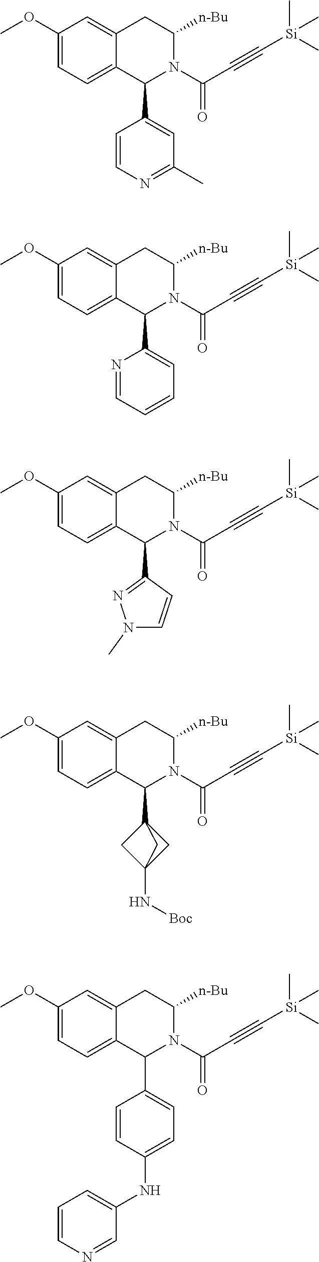

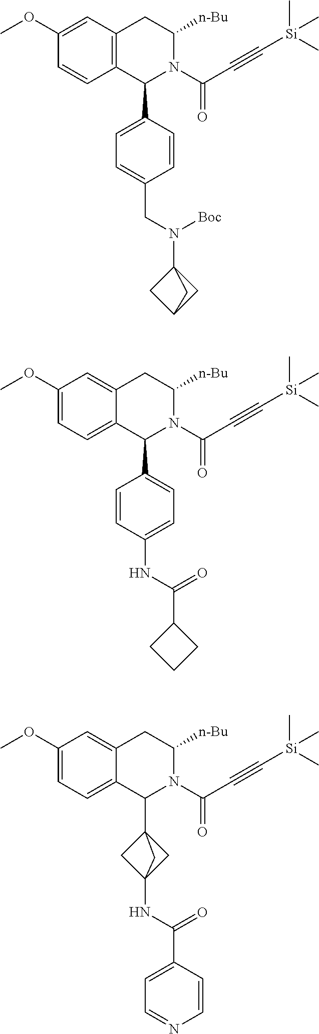

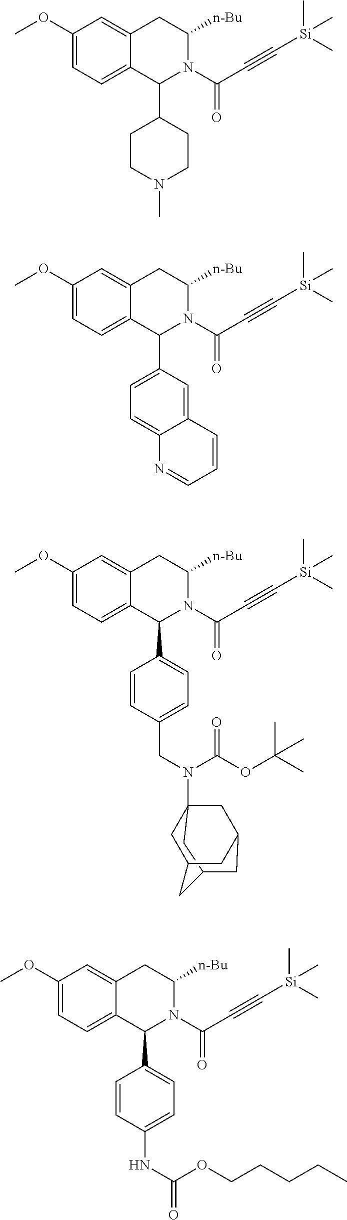

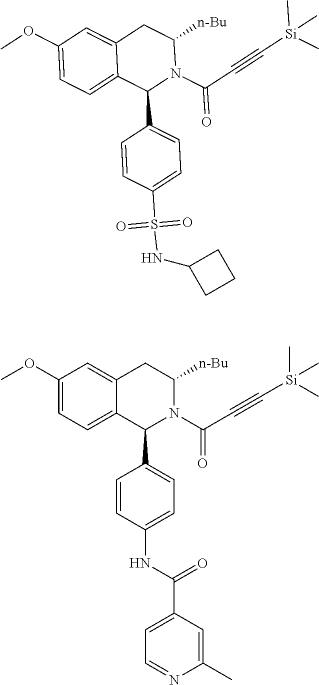

10. The compound of claim 9, or a tautomer, stereoisomer, mixture of stereoisomers, isotopically enriched analog, or pharmaceutically acceptable salt thereof, represented by formula A-IL

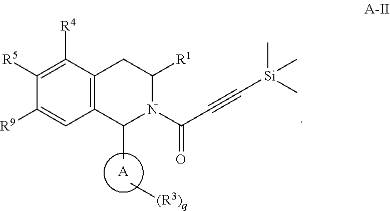

11. The compound of any one of claims 1-10, wherein R⁴ and R⁵ are each independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆ alkyl, C₂-C₆ alkenyl, C₂-C₆alkynyl, or C₃-C₁₀ cycloalkyl, wherein each C₁-C₆ alkyl, C₂-C₆ alkenyl, C₂-C₆alkynyl, or C₃-C₁₀ cycloalkyl of R⁴ is independently unsubstituted or substituted with one to three R¹⁰.

12. The compound of claim 11, wherein R⁴ and R⁵ are each independently hydrogen, halo, —CN, —OH, —OR⁸, C₁-C₆ alkyl, C₂-C₆alkynyl, or C₃-C₁₀ cycloalkyl, wherein each C₁-C₆ alkyl, C₂-C₆alkynyl, or C₃-C₁₀ cycloalkyl of R⁴ is independently unsubstituted or substituted with one to three R¹⁰.

13. The compound of claim 1, or a tautomer, stereoisomer, mixture of stereoisomers, isotopically enriched analog, or pharmaceutically acceptable salt thereof, represented by formula A-III. 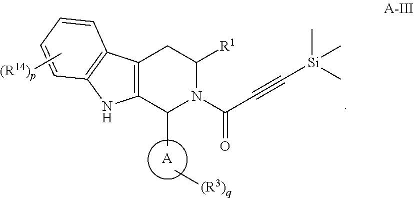
wherein p is 0, 1, 2, or 3.

14. The compound of claim 13, wherein p is 0.

15. The compound of claim 13, wherein p is 1, 2, or 3.

16. The compound of claim 15, wherein each R¹⁴ is independently halo, —CN, —OH, —OR⁸, C₁-C₆ alkyl, C₂-C₆alkynyl, or C₃-C₁₀ cycloalkyl.

17. The compound of any one of claims 1-16, wherein R¹ is C₁-C₆ alkyl.

18. The compound of claim 17, wherein R¹ is n-butyl or i-butyl.

19. The compound of any one of claims 1-16, wherein R¹ is C₃-C₁₀ cycloalkyl or —C₁-C₆ alkyl-C₃-C₁₀ cycloalkyl.

20. The compound of any one of claims 1-19, wherein ring A is C₄-C₁₀ cycloalkyl.

21. The compound of any one of claims 1-19, wherein ring A is heterocyclyl.

22. The compound of any one of claims 1-19, wherein ring A is aryl.

23. The compound of claim 22, wherein ring A is phenyl.

24. The compound of any one of claims 1-19, wherein ring A is heteroaryl.

25. The compound of any one of claims 1-24, wherein q is 1, 2, or 3.

26. The compound of any one of claims 1-25, wherein at least one R³ is —NHR⁸, —OR⁸, —S(O)₂R⁸, —S(O)R⁸, —NR¹²C(O)R⁸, —NR¹²C(O)OR⁸, —OC(O)R⁸, or —OC(O)CHR⁸N(R¹²)₂.

27. The compound of claim 26, wherein at least one R³ is —NHR⁸ or —OR⁸.

28. The compound of claim 26 or 27, wherein R⁸ is C₃-C₁₀ cycloalkyl.

29. The compound of claim 28, wherein R⁸ is adamantyl.

30. The compound of any one of claims 1-29, wherein q is 2, and one R³ is halo or —CN, and the other R³ is —N(R⁸)₂.

31. The compound of any one of claims 1-24, wherein q is 0.

32. A compound of Table A-1A or Table A-2A, or a tautomer, stereoisomer, mixture of stereoisomers, isotopically enriched analog, or pharmaceutically acceptable salt thereof.

33. A compound, or a tautomer, stereoisomer, mixture of stereoisomers, isotopically enriched analog, or pharmaceutically acceptable salt thereof, represented by Formula B-I, as in Table B-1: 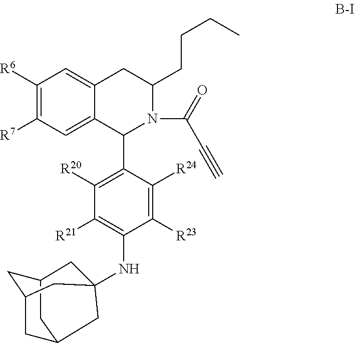
TABLE B-1

| Compound No. | R⁶ | R⁷ | R²⁰ | R²¹ | R²³ | R²⁴ |
|---|---|---|---|---|---|---|
| B-I-1 | —OCH₃ | —H | —CN | —H | —H | —H |
| B-I-2 | —OCF₃ | —H | —CN | —H | —H | —H |
| B-I-3 | —CF₃ | —H | —CN | —H | —H | —H |
| B-I-4 | —CN | —H | —CN | —H | —H | —H |
| B-I-5 | —F | —H | —CN | —H | —H | —H |
| B-I-6 | —CF₂H | —H | —CN | —H | —H | —H |
| B-I-7 | —SO₂CH₃ | —H | —CN | —H | —H | —H |
| B-1-8 | —F | —F | —CN | —H | —H | —H |
| B-I-9 | —CN | —F | —CN | —H | —H | —H |
| B-I-10 | —F | —CN | —CN | —H | —H | —H |
| B-I-11 | —CF₃ | —F | —CN | —H | —H | —H |
| B-I-12 | —F | —CF₃ | —CN | —H | —H | —H |
| B-I-13 | —OCF₃ | —F | —CN | —H | —H | —H |
| B-I-14 | —F | —OCF₃ | —CN | —H | —H | —H |
| B-I-15 | —OCH₃ | —H | —CN | —F | —H | —H |
| B-I-16 | —OCF₃ | —H | —CN | —F | —H | —H |
| B-I-17 | —CF₃ | —H | —CN | —F | —H | —H |
| B-I-18 | —CN | —H | —CN | —F | —H | —H |
| B-I-19 | —F | —H | —CN | —F | —H | —H |
| B-I-20 | —CF₂H | —H | —CN | —F | —H | —H |
| B-I-21 | —SO₂CH₃ | —H | —CN | —F | —H | —H |
| B-I-22 | —F | —F | —CN | —F | —H | —H |
| B-I-23 | —CN | —F | —CN | —F | —H | —H |
| B-I-24 | —F | —CN | —CN | —F | —H | —H |
| B-I-25 | —CF₃ | —F | —CN | —F | —H | —H |
| B-I-26 | —F | —CF₃ | —CN | —F | —H | —H |
| B-I-27 | —OCF₃ | —F | —CN | —F | —H | —H |
| B-I-28 | —F | —OCF₃ | —CN | —F | —H | —H |
| B-I-29 | —OCH₃ | —H | —CN | —H | —F | —H |
| B-I-30 | —OCF₃ | —H | —CN | —H | —F | —H |
| B-I-31 | —CF₃ | —H | —CN | —H | —F | —H |
| B-I-32 | —CN | —H | —CN | —H | —F | —H |
| B-I-33 | —F | —H | —CN | —H | —F | —H |
| B-I-34 | —CF₂H | —H | —CN | —H | —F | —H |
| B-I-35 | —SO₂CH₃ | —H | —CN | —H | —F | —H |
| B-I-36 | —F | —F | —CN | —H | —F | —H |
| B-I-37 | —CN | —F | —CN | —H | —F | —H |
| B-I-38 | —F | —CN | —CN | —H | —F | —H |
| B-I-39 | —CF₃ | —F | —CN | —H | —F | —H |
| B-I-40 | —F | —CF₃ | —CN | —H | —F | —H |
| B-I-41 | —OCF₃ | —F | —CN | —H | —F | —H |
| B-I-42 | —F | —OCF₃ | —CN | —H | —F | —H |
| B-I-43 | —OCH₃ | —H | —CN | —H | —H | —F |
| B-I-44 | —OCF₃ | —H | —CN | —H | —H | —F |
| B-I-45 | —CF₃ | —H | —CN | —H | —H | —F |
| B-I-46 | —CN | —H | —CN | —H | —H | —F |
| B-I-47 | —F | —H | —CN | —H | —H | —F |
| B-I-48 | —CF₂H | —H | —CN | —H | —H | —F |
| B-I-49 | —SO₂CH₃ | —H | —CN | —H | —H | —F |
| B-I-50 | —F | —F | —CN | —H | —H | —F |
| B-I-51 | —CN | —F | —CN | —H | —H | —F |
| B-I-52 | —F | —CN | —CN | —H | —H | —F |
| B-I-53 | —CF₃ | —F | —CN | —H | —H | —F |
| B-I-54 | —F | —CF₃ | —CN | —H | —H | —F |
| B-I-55 | —OCF₃ | —F | —CN | —H | —H | —F |
| B-I-56 | —F | —OCF₃ | —CN | —H | —H | —F |
| B-I-57 | —OCH₃ | —H | —H | —CN | —H | —H |
| B-I-58 | —OCF₃ | —H | —H | —CN | —H | —H |
| B-I-59 | —CF₃ | —H | —H | —CN | —H | —H |
| B-I-60 | —CN | —H | —H | —CN | —H | —H |
| B-I-61 | —F | —H | —H | —CN | —H | —H |
| B-I-62 | —CF₂H | —H | —H | —CN | —H | —H |
| B-I-63 | —SO₂CH₃ | —H | —H | —CN | —H | —H |
| B-I-64 | —F | —F | —H | —CN | —H | —H |
| B-I-65 | —CN | —F | —H | —CN | —H | —H |
| B-I-66 | —F | —CN | —H | —CN | —H | —H |
| B-I-67 | —CF₃ | —F | —H | —CN | —H | —H |
| B-I-68 | —F | —CF₃ | —H | —CN | —H | —H |
| B-I-69 | —OCF₃ | —F | —H | —CN | —H | —H |
| B-I-70 | —F | —OCF3 | —H | —CN | —H | —H |
| B-I-71 | —OCH₃ | —H | —F | —CN | —H | —H |
| B-I-72 | —OCF₃ | —H | —F | —CN | —H | —H |
| B-I-73 | —CF₃ | —H | —F | —CN | —H | —H |
| B-I-74 | -CN | —H | —F | —CN | —H | —H |
| B-I-75 | —F | —H | —F | —CN | —H | —H |
| B-I-76 | —CF₂H | —H | —F | —CN | —H | —H |
| B-I-77 | —SO₂CH₃ | —H | —F | —CN | —H | —H |
| B-I-78 | —F | —F | —F | —CN | —H | —H |
| B-I-79 | —CN | —F | —F | —CN | —H | —H |
| B-I-80 | —F | —CN | —F | —CN | —H | —H |
| B-I-81 | —CF₃ | —F | —F | —CN | —H | —H |
| B-I-82 | —F | —CF₃ | —F | —CN | —H | —H |
| B-I-83 | —OCF₃ | —F | —F | —CN | —H | —H |
| B-I-84 | —F | —OCF₃ | —F | —CN | —H | —H |
| B-I-85 | —OCH₃ | —H | —H | —CN | —F | —H |
| B-I-86 | —OCF₃ | —H | —H | —CN | —F | —H |
| B-I-87 | —CF₃ | —H | —H | —CN | —F | —H |
| B-I-88 | —CN | —H | —H | —CN | —F | —H |
| B-I-89 | —F | —H | —H | —CN | —F | —H |
| B-I-90 | —CF₂H | —H | —H | —CN | —F | —H |
| B-I-91 | —SO₂CH₃ | —H | —H | —CN | —F | —H |
| B-I-92 | —F | —F | —H | —CN | —F | —H |
| B-I-93 | —CN | —F | —H | —CN | —F | —H |
| B-I-94 | —F | —CN | —H | —CN | —F | —H |
| B-I-95 | —CF₃ | —F | —H | —CN | —F | —H |
| B-I-96 | —F | —CF₃ | —H | —CN | —F | —H |
| B-I-97 | —OCF₃ | —F | —H | —CN | —F | —H |
| B-I-98 | —F | —OCF₃ | —H | —CN | —F | —H |
| B-I-99 | —OCH₃ | —H | —H | —CN | —H | —F |
| B-I-100 | —OCF₃ | —H | —H | —CN | —H | —F |
| B-I-101 | —CF₃ | —H | —H | —CN | —H | —F |
| B-I-102 | —CN | —H | —H | —CN | —H | —F |
| B-I-103 | —F | —H | —H | —CN | —H | —F |
| B-I-104 | —CF₂H | —H | —H | —CN | —H | —F |
| B-I-105 | —SO₂CH₃ | —H | —H | —CN | —H | —F |
| B-I-106 | —F | —F | —H | —CN | —H | —F |
| B-I-107 | —CN | —F | —H | —CN | —H | —F |
| B-I-108 | —F | —CN | —H | —CN | —H | —F |
| B-I-109 | —CF₃ | —F | —H | —CN | —H | —F |
| B-I-110 | —F | —CF₃ | —H | —CN | —H | —F |
| B-I-111 | —OCF3 | —F | —H | —CN | —H | —F |
| B-I-112 | —F | —OCF₃ | —H | —CN | —H | —F |
| B-I-113 | —OCH₃ | —H | —F | —H | —H | —H |
| B-I-114 | —OCF₃ | —H | —F | —H | —H | —H |
| B-I-115 | —CF₃ | —H | —F | —H | —H | —H |
| B-I-116 | —CN | —H | —F | —H | —H | —H |
| B-I-117 | —F | —H | —F | —H | —H | —H |
| B-I-118 | —CF₂H | —H | —F | —H | —H | —H |
| B-I-119 | —SO₂CH₃ | —H | —F | —H | —H | —H |
| B-I-120 | —F | —F | —F | —H | —H | —H |
| B-I-121 | —CN | —F | —F | —H | —H | —H |
| B-I-122 | —F | —CN | —F | —H | —H | —H |
| B-I-123 | —CF₃ | —F | —F | —H | —H | —H |
| B-I-124 | —F | —CF₃ | —F | —H | —H | —H |
| B-I-125 | —OCF₃ | —F | —F | —H | —H | —H |
| B-I-126 | —F | —OCF₃ | —F | —H | —H | —H |
| B-I-127 | —OCH₃ | —H | —H | —F | —H | —H |
| B-I-128 | —OCF₃ | —H | —H | —F | —H | —H |
| B-I-129 | —CF₃ | —H | —H | —F | —H | —H |
| B-I-130 | —CN | —H | —H | —F | —H | —H |
| B-I-131 | —F | —H | —H | —F | —H | —H |
| B-I-132 | —CF₂H | —H | —H | —F | —H | —H |
| B-I-133 | —SO₂CH₃ | —H | —H | —F | —H | —H |
| B-I-134 | —F | —F | —H | —F | —H | —H |
| B-I-135 | —CN | —F | —H | —F | —H | —H |
| B-I-136 | —F | —CN | —H | —F | —H | —H |
| B-I-137 | —CF₃ | —F | —H | —F | —H | —H |
| B-I-138 | —F | —CF₃ | —H | —F | —H | —H |
| B-I-139 | —OCF₃ | —F | —H | —F | —H | —H |
| B-I-140 | —F | —OCF₃ | —H | —F | —H | —H |
| B-I-141 | —OCH₃ | —H | —F | —F | —H | —H |
| B-I-142 | —OCF₃ | —H | —F | —F | —H | —H |
| B-I-143 | —CF₃ | —H | —F | —F | —H | —H |
| B-I-144 | —CN | —H | —F | —F | —H | —H |
| B-I-145 | —F | —H | —F | —F | —H | —H |
| B-I-146 | —CF₂H | —H | —F | —F | —H | —H |
| B-I-147 | —SO₂CH₃ | —H | —F | —F | —H | —H |
| B-I-148 | —F | —F | —F | —F | —H | —H |
| B-I-149 | —CN | —F | —F | —F | —H | —H |
| B-I-150 | —F | —CN | —F | —F | —H | —H |
| B-I-151 | —CF₃ | —F | —F | —F | —H | —H |
| B-I-152 | —F | —CF₃ | —F | —F | —H | —H |
| B-I-153 | —OCF₃ | —F | —F | —F | —H | —H |
| B-I-154 | —F | —OCF₃ | —F | —F | —H | —H |
| B-I-155 | —OCH₃ | —H | —F | —H | —F | —H |
| B-I-156 | —OCF₃ | —H | —F | —H | —F | —H |
| B-I-157 | —CF₃ | —H | —F | —H | —F | —H |
| B-I-158 | —CN | —H | —F | —H | —F | —H |
| B-I-159 | —F | —H | —F | —H | —F | —H |
| B-I-160 | —CF₂H | —H | —F | —H | —F | —H |
| B-I-161 | —SO₂CH₃ | —H | —F | —H | —F | —H |
| B-I-162 | —F | —F | —F | —H | —F | —H |
| B-I-163 | —CN | —F | —F | —H | —F | —H |
| B-I-164 | —F | —CN | —F | —H | —F | —H |
| B-I-165 | —CF₃ | —F | —F | —H | —F | —H |
| B-I-166 | —F | —CF₃ | —F | —H | —F | —H |
| B-I-167 | —OCF3 | —F | —F | —H | —F | —H |
| B-I-168 | —F | —OCF₃ | —F | —H | —F | —H |
| B-I-169 | —OCH₃ | —H | —F | —H | —H | —F |
| B-I-170 | —OCF₃ | —H | —F | —H | —H | —F |
| B-I-171 | —CF₃ | —H | —F | —H | —H | —F |
| B-I-172 | —CN | —H | —F | —H | —H | —F |
| B-I-173 | —F | —H | —F | —H | —H | —F |
| B-I-174 | —CF₂H | —H | —F | —H | —H | —F |
| B-I-175 | —SO₂CH₃ | —H | —F | —H | —H | —F |
| B-I-176 | —F | —F | —F | —H | —H | —F |
| B-I-177 | —CN | —F | —F | —H | —H | —F |
| B-I-178 | —F | —CN | —F | —H | —H | —F |
| B-I-179 | —CF₃ | —F | —F | —H | —H | —F |
| B-I-180 | —F | —CF₃ | —F | —H | —H | —F |
| B-I-181 | —OCF₃ | —F | —F | —H | —H | —F |
| B-I-182 | —F | —OCF₃ | —F | —H | —H | —F |
| B-I-183 | —OCH₃ | —H | —H | —F | —F | —H |
| B-I-184 | —OCF₃ | —H | —H | —F | —F | —H |
| B-I-185 | —CF₃ | —H | —H | —F | —F | —H |
| B-I-186 | —CN | —H | —H | —F | —F | —H |
| B-I-187 | —F | —H | —H | —F | —F | —H |
| B-I-188 | —CF₂H | —H | —H | —F | —F | —H |
| B-I-189 | —SO₂CH₃ | —H | —H | —F | —F | —H |
| B-I-190 | —F | —F | —H | —F | —F | —H |
| B-I-191 | —CN | —F | —H | —F | —F | —H |
| B-I-192 | —F | —CN | —H | —F | —F | —H |
| B-I-193 | —CF₃ | —F | —H | —F | —F | —H |
| B-I-194 | —F | —CF₃ | —H | —F | —F | —H |
| B-I-195 | —OCF₃ | —F | —H | —F | —F | —H |
| B-I-196 | —F | —OCF₃ | —H | —F | —F | —H |
| B-I-197 | —OCH₃ | —H | —CF₃ | —H | —H | —H |
| B-I-198 | —OCF₃ | —H | —CF₃ | —H | —H | —H |
| B-I-199 | —CF₃ | —H | —CF₃ | —H | —H | —H |
| B-I-200 | —CN | —H | —CF₃ | —H | —H | —H |
| B-I-201 | —F | —H | —CF₃ | —H | —H | —H |
| B-I-202 | —CF₂H | —H | —CF₃ | —H | —H | —H |
| B-I-203 | —SO₂CH₃ | —H | —CF₃ | —H | —H | —H |
| B-I-204 | —F | —F | —CF₃ | —H | —H | —H |
| B-I-205 | —CN | —F | —CF₃ | —H | —H | —H |
| B-I-206 | —F | —CN | —CF₃ | —H | —H | —H |
| B-I-207 | —CF₃ | —F | —CF₃ | —H | —H | —H |
| B-I-208 | —F | —CF₃ | —CF₃ | —H | —H | —H |
| B-I-209 | —OCF₃ | —F | —CF₃ | —H | —H | —H |
| B-I-210 | —F | —OCF₃ | —CF₃ | —H | —H | —H |
| B-I-211 | —OCH₃ | —H | —CF₃ | —F | —H | —H |
| B-I-212 | —OCF3 | —H | —CF₃ | —F | —H | —H |
| B-I-213 | —CF₃ | —H | —CF₃ | —F | —H | —H |
| B-I-214 | —CN | —H | —CF₃ | —F | —H | —H |
| B-I-215 | —F | —H | —CF₃ | —F | —H | —H |
| B-I-216 | —CF₂H | —H | —CF₃ | —F | —H | —H |
| B-I-217 | —SO₂CH₃ | —H | —CF₃ | —F | —H | —H |
| B-I-218 | —F | —F | —CF₃ | —F | —H | —H |
| B-I-219 | —CN | —F | —CF₃ | —F | —H | —H |
| B-I-220 | —F | —CN | —CF₃ | —F | —H | —H |
| B-I-221 | —CF₃ | —F | —CF₃ | —F | —H | —H |
| B-I-222 | —F | —CF₃ | —CF₃ | —F | —H | —H |
| B-I-223 | —OCF₃ | —F | —CF₃ | —F | —H | —H |
| B-I-224 | —F | —OCF₃ | —CF₃ | —F | —H | —H |
| B-I-225 | —OCH₃ | —H | —CF₃ | —H | —F | —H |
| B-I-226 | —OCF₃ | —H | —CF₃ | —H | —F | —H |
| B-I-227 | —CF₃ | —H | —CF₃ | —H | —F | —H |
| B-I-228 | —CN | —H | —CF₃ | —H | —F | —H |
| B-I-229 | —F | —H | —CF₃ | —H | —F | —H |
| B-I-230 | —CF₂H | —H | —CF₃ | —H | —F | —H |
| B-I-231 | —SO₂CH₃ | —H | —CF₃ | —H | —F | —H |
| B-I-232 | —F | —F | —CF₃ | —H | —F | —H |
| B-I-233 | —CN | —F | —CF₃ | —H | —F | —H |
| B-I-234 | —F | —CN | —CF₃ | —H | —F | —H |
| B-I-235 | —CF₃ | —F | —CF₃ | —H | —F | —H |
| B-I-236 | —F | —CF₃ | —CF₃ | —H | —F | —H |
| B-I-237 | —OCF₃ | —F | —CF₃ | —H | —F | —H |
| B-I-238 | —F | —OCF₃ | —CF₃ | —H | —F | —H |
| B-I-239 | —OCH₃ | —H | —CF₃ | —H | —H | —F |
| B-I-240 | —OCF₃ | —H | —CF₃ | —H | —H | —F |
| B-I-241 | —CF₃ | —H | —CF₃ | —H | —H | —F |
| B-I-242 | —CN | —H | —CF₃ | —H | —H | —F |
| B-I-243 | —F | —H | —CF₃ | —H | —H | —F |
| B-I-244 | —CF₂H | —H | —CF₃ | —H | —H | —F |
| B-I-245 | —SO₂CH₃ | —H | —CF₃ | —H | —H | —F |
| B-I-246 | —F | —F | —CF₃ | —H | —H | —F |
| B-I-247 | —CN | —F | —CF₃ | —H | —H | —F |
| B-I-248 | —F | —CN | —CF₃ | —H | —H | —F |
| B-I-249 | —CF₃ | —F | —CF₃ | —H | —H | —F |
| B-I-250 | —F | —CF3 | —CF₃ | —H | —H | —F |
| B-I-251 | —OCF₃ | —F | —CF₃ | —H | —H | —F |
| B-I-252 | —F | —OCF₃ | —CF₃ | —H | —H | —F |
| B-I-253 | —OCH₃ | —H | —F | —CF₃ | —H | —H |
| B-I-254 | —OCF₃ | —H | —F | —CF₃ | —H | —H |
| B-I-255 | —CF₃ | —H | —F | —CF₃ | —H | —H |
| B-I-256 | —CN | —H | —F | —CF₃ | —H | —H |
| B-I-257 | —F | —H | —F | —CF₃ | —H | —H |
| B-I-258 | —CF₂H | —H | —F | —CF₃ | —H | —H |
| B-I-259 | —SO₂CH₃ | —H | —F | —CF₃ | —H | —H |
| B-I-260 | —F | —F | —F | —CF₃ | —H | —H |
| B-I-261 | —CN | —F | —F | —CF₃ | —H | —H |
| B-I-262 | —F | —CN | —F | —CF₃ | —H | —H |
| B-I-263 | —CF₃ | —F | —F | —CF₃ | —H | —H |
| B-I-264 | —F | —CF₃ | —F | —CF₃ | —H | —H |
| B-I-265 | —OCF₃ | —F | —F | —CF₃ | —H | —H |
| B-I-266 | —F | —OCF₃ | —F | —CF₃ | —H | —H |
| B-I-267 | —OCH₃ | —H | —H | —CF₃ | —F | —H |
| B-I-268 | —OCF₃ | —H | —H | —CF₃ | —F | —H |
| B-I-269 | —CF₃ | —H | —H | —CF₃ | —F | —H |
| B-I-270 | —CN | —H | —H | —CF₃ | —F | —H |
| B-I-271 | —F | —H | —H | —CF₃ | —F | —H |
| B-I-272 | —CF₂H | —H | —H | —CF₃ | —F | —H |
| B-I-273 | —SO₂CH₃ | —H | —H | —CF₃ | —F | —H |
| B-I-274 | —F | —F | —H | —CF₃ | —F | —H |
| B-I-275 | —CN | —F | —H | —CF₃ | —F | —H |
| B-I-276 | —F | —CN | —H | —CF₃ | —F | —H |
| B-I-277 | —CF₃ | —F | —H | —CF₃ | —F | —H |
| B-I-278 | —F | —CF₃ | —H | —CF₃ | —F | —H |
| B-I-279 | —OCF₃ | —F | —H | —CF₃ | —F | —H |
| B-I-280 | —F | —OCF₃ | —H | —CF₃ | —F | —H |
| B-I-281 | —OCH₃ | —H | —H | —CF₃ | —H | —F |
| B-I-282 | —OCF₃ | —H | —H | —CF₃ | —H | —F |
| B-I-283 | —CF₃ | —H | —H | —CF₃ | —H | —F |
| B-I-284 | —CN | —H | —H | —CF₃ | —H | —F |
| B-I-285 | —F | —H | —H | —CF₃ | —H | —F |
| B-I-286 | —CF₂H | —H | —H | —CF₃ | —H | —F |
| B-I-287 | —SO₂CH₃ | —H | —H | —CF₃ | —H | —F |
| B-I-288 | —F | —F | —H | —CF₃ | —H | —F |
| B-I-289 | —CN | —F | —H | —CF₃ | —H | —F |
| B-I-290 | —F | —CN | —H | —CF₃ | —H | —F |
| B-I-291 | —CF₃ | —F | —H | —CF₃ | —H | —F |
| B-I-292 | —F | —CF₃ | —H | —CF₃ | —H | —F |
| B-I-293 | —OCF₃ | —F | —H | —CF₃ | —H | —F |
| B-I-294 | —F | —OCF₃ | —H | —CF₃ | —H | —F |

34. A compound, or a tautomer, stereoisomer, mixture of stereoisomers, isotopically enriched analog, or pharmaceutically acceptable salt thereof, represented by Formula B-IA, as in Table B-2: 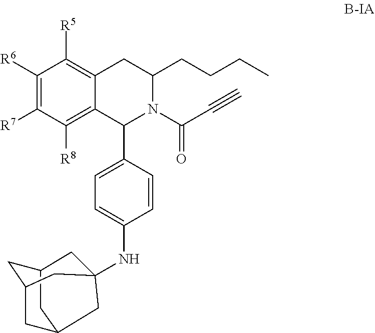
TABLE B-2

| Compound No. | R⁵ | R⁶ | R⁷ | R⁸ |
|---|---|---|---|---|
| B-IA-1 | —OCH₃ | —H | —H | —H |
| B-IA-2 | —H | —OCH₃ | —H | —H |
| B-IA-3 | —H | —H | —OCH₃ | —H |
| B-IA-4 | —H | —H | —H | —OCH₃ |
| B-IA-5 | —OCF₃ | —H | —H | —H |
| B-IA-6 | —H | —OCF₃ | —H | —H |
| B-IA-7 | —H | —H | —OCF₃ | —H |
| B-IA-8 | —H | —H | —H | —OCF₃ |
| B-IA-9 | —CF₃ | —H | —H | —H |
| B-IA-10 | —H | —CF₃ | —H | —H |
| B-IA-11 | —H | —H | —CF₃ | —H |
| B-IA-12 | —H | —H | —H | —CF₃ |
| B-IA-13 | —CN | —H | —H | —H |
| B-IA-14 | —H | —CN | —H | —H |
| B-IA-15 | —H | —H | —CN | —H |
| B-IA-16 | —H | —H | —H | —CN |
| B-IA-17 | —F | —H | —H | —H |
| B-IA-18 | —H | —F | —H | —H |
| B-IA-19 | —H | —H | —F | —H |
| B-IA-20 | —H | —H | —H | —F |
| B-IA-21 | —CF₂H | —H | —H | —H |
| B-IA-22 | —H | —CF₂H | —H | —H |
| B-IA-23 | —H | —H | —CF₂H | —H |
| B-IA-24 | —H | —H | —H | —CF₂H |
| B-IA-25 | —SO₂CH₃ | —H | —H | —H |
| B-IA-26 | —H | —SO₂CH₃ | —H | —H |
| B-IA-27 | —H | —H | —SO₂CH₃ | —H |
| B-IA-28 | —H | —H | —H | —SO₂CH₃ |
| B-IA-29 | —SO₂NHCH₃ | —H | —H | —H |
| B-IA-30 | —H | —SO₂NHCH₃ | —H | —H |
| B-IA-31 | —H | —H | —SO₂NHCH₃ | —H |
| B-IA-32 | —H | —H | —H | —SO₂NHCH₃ |

35. A compound, or a tautomer, stereoisomer, mixture of stereoisomers, isotopically enriched analog, or pharmaceutically acceptable salt thereof, represented by Formula B-IB, as in Table B-3: 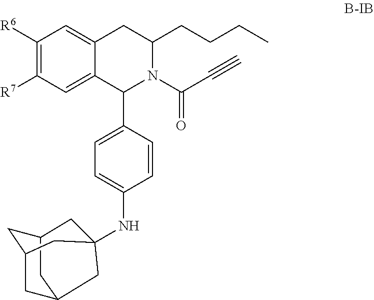
TABLE B-3

| Compound No. | R⁶ | R ⁷ |
|---|---|---|
| B-IB-1 | —OCH₃ | —F |
| B-IB-2 | —F | —OCH₃ |
| B-IB-3 | —OCF₃ | —F |
| B-IB-4 | —F | —OCF₃ |
| B-IB-5 | —F | —F |
| B-IB-6 | —OCH₃ | —CN |
| B-IB-7 | —CN | —OCH₃ |
| B-IB-8 | —SO₂CH₃ | —F |
| B-IB-9 | —F | —SO₂CH₃ |
| B-IB-10 | —CN | —F |
| B-IB-11 | —F | —CN |

36. A compound, or a tautomer, stereoisomer, mixture of stereoisomers, isotopically enriched analog, or pharmaceutically acceptable salt thereof, represented by Formula B-II, as in Table B-4: 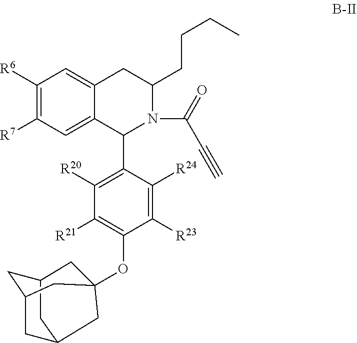
TABLE B-4

| Compound | | | | | | |
|---|---|---|---|---|---|---|
| No. | R⁶ | R⁷ | R²⁰ | R²¹ | R²³ | R²⁴ |
| B-II-1 | —OCH₃ | —H | —CN | —H | —H | —H |
| B-II-2 | —OCF₃ | —H | —CN | —H | —H | —H |
| B-II-3 | —CF₃ | —H | —CN | —H | —H | —H |
| B-II-4 | —CN | —H | —CN | —H | —H | —H |
| B-II-5 | —F | —H | —CN | —H | —H | —H |
| B-II-6 | —CF₂H | —H | —CN | —H | —H | —H |
| B-II-7 | —SO₂CH₃ | —H | —CN | —H | —H | —H |
| B-II-8 | —F | —F | —CN | —H | —H | —H |
| B-II-9 | —CN | —F | —CN | —H | —H | —H |
| B-II-10 | —F | —CN | —CN | —H | —H | —H |
| B-II-11 | —CF₃ | —F | —CN | —H | —H | —H |
| B-II-12 | —F | —CF₃ | —CN | —H | —H | —H |
| B-II-13 | —OCF₃ | —F | —CN | —H | —H | —H |
| B-II-14 | —F | —OCF₃ | —CN | —H | —H | —H |
| B-II-15 | —OCH₃ | —H | —CN | —F | —H | —H |
| B-II-16 | —OCF₃ | —H | —CN | —F | —H | —H |
| B-II-17 | —CF₃ | —H | —CN | —F | —H | —H |
| B-II-18 | —CN | —H | —CN | —F | —H | —H |
| B-II-19 | —F | —H | —CN | —F | —H | —H |
| B-II-20 | —CF₂H | —H | —CN | —F | —H | —H |
| B-II-21 | —SO₂CH₃ | —H | —CN | —F | —H | —H |
| B-II-22 | —F | —F | —CN | —F | —H | —H |
| B-II-23 | —CN | —F | —CN | —F | —H | —H |
| B-II-24 | —F | —CN | —CN | —F | —H | —H |
| B-II-25 | —CF₃ | —F | —CN | —F | —H | —H |
| B-II-26 | —F | —CF | —CN | —F | —H | —H |
| B-II-27 | —OCF₃ | —F | —CN | —F | —H | —H |
| B-II-28 | —F | —OCF₃ | —CN | —F | —H | —H |
| B-II-29 | —OCH₃ | —H | —CN | —H | —F | —H |
| B-II-30 | —OCF₃ | —H | —CN | —H | —F | —H |
| B-II-31 | —CF₃ | —H | —CN | —H | —F | —H |
| B-II-32 | —CN | —H | —CN | —H | —F | —H |
| B-II-33 | —F | —H | —CN | —H | —F | —H |
| B-II-34 | —CF₂H | —H | —CN | —H | —F | —H |
| B-II-35 | —SO₂CH₃ | —H | —CN | —H | —F | —H |
| B-II-36 | —F | —F | —CN | —H | —F | —H |
| B-II-37 | —CN | —F | —CN | —H | —F | —H |
| B-II-38 | —F | —CN | —CN | —H | —F | —H |
| B-II-39 | —CF₃ | —F | —CN | —H | —F | —H |
| B-II-40 | —F | —CF₃ | —CN | —H | —F | —H |
| B-II-41 | —OCF₃ | —F | —CN | —H | —F | —H |
| B-II-42 | —F | —OCF₃ | —CN | —H | —F | —H |
| B-II-43 | —OCH₃ | —H | —CN | —H | —H | —F |
| B-II-44 | —OCF₃ | —H | —CN | —H | —H | —F |
| B-II-45 | —CF₃ | —H | —CN | —H | —H | —F |
| B-II-46 | —CN | —H | —CN | —H | —H | —F |
| B-II-47 | —F | —H | —CN | —H | —H | —F |
| B-II-48 | —CF₂H | —H | —CN | —H | —H | —F |
| B-II-49 | —SO₂CH₃ | —H | —CN | —H | —H | —F |
| B-II-50 | —F | —F | —CN | —H | —H | —F |
| B-II-51 | —CN | —F | —CN | —H | —H | —F |
| B-II-52 | —F | —CN | —CN | —H | —H | —F |
| B-II-53 | —CF₃ | —F | —CN | —H | —H | —F |
| B-II-54 | —F | —CF₃ | —CN | —H | —H | —F |
| B-II-55 | —OCF₃ | —F | —CN | —H | —H | —F |
| B-II-56 | —F | —OCF₃ | —CN | —H | —H | —F |
| B-II-57 | —OCH₃ | —H | —H | —CN | —H | —H |
| B-II-58 | —OCF₃ | —H | —H | —CN | —H | —H |
| B-II-59 | —CF₃ | —H | —H | —CN | —H | —H |
| B-II-60 | —CN | —H | —H | —CN | —H | —H |
| B-II-61 | —F | —H | —H | —CN | —H | —H |
| B-II-62 | —CF₂H | —H | —H | —CN | —H | —H |
| B-II-63 | —SO₂CH₃ | —H | —H | —CN | —H | —H |
| B-II-64 | —F | —F | —H | —CN | —H | —H |
| B-II-65 | —CN | —F | —H | —CN | —H | —H |
| B-II-66 | —F | —CN | —H | —CN | —H | —H |
| B-II-67 | —CF₃ | —F | —H | —CN | —H | —H |
| B-II-68 | —F | —CF₃ | —H | —CN | —H | —H |
| B-II-69 | —OCF₃ | —F | —H | —CN | —H | —H |
| B-II-70 | —F | —OCF₃ | —H | —CN | —H | —H |
| B-II-71 | —OCH₃ | —H | —F | —CN | —H | —H |
| B-II-72 | —OCF₃ | —H | —F | —CN | —H | —H |
| B-II-73 | —CF₃ | —H | —F | —CN | —H | —H |
| B-II-74 | —CN | —H | —F | —CN | —H | —H |
| B-II-75 | —F | —H | —F | —CN | —H | —H |
| B-II-76 | —CF₂H | —H | —F | —CN | —H | —H |
| B-II-77 | —SO₂CH₃ | —H | —F | —CN | —H | —H |
| B-II-78 | —F | —F | —F | —CN | —H | —H |
| B-II-79 | —CN | —F | —F | —CN | —H | —H |
| B-II-80 | —F | —CN | —F | —CN | —H | —H |
| B-II-81 | —CF₃ | —F | —F | —CN | —H | —H |
| B-II-82 | —F | —CF₃ | —F | —CN | —H | —H |
| B-II-83 | —OCF₃ | —F | —F | —CN | —H | —H |
| B-II-84 | —F | —OCF₃ | —F | —CN | —H | —H |
| B-II-85 | —OCH₃ | —H | —H | —CN | —F | —H |
| B-II-86 | —OCF₃ | —H | —H | —CN | —F | —H |
| B-II-87 | —CF₃ | —H | —H | —CN | —F | —H |
| B-II-88 | —CN | —H | —H | —CN | —F | —H |
| B-II-89 | —F | —H | —H | —CN | —F | —H |
| B-II-90 | —CF₂H | —H | —H | —CN | —F | —H |
| B-II-91 | —SO₂CH₃ | —H | —H | —CN | —F | —H |
| B-II-92 | —F | —F | —H | —CN | —F | —H |
| B-II-93 | —CN | —F | —H | —CN | —F | —H |
| B-II-94 | —F | —CN | —H | —CN | —F | —H |
| B-II-95 | —CF₃ | —F | —H | —CN | —F | —H |
| B-II-96 | —F | —CF₃ | —H | —CN | —F | —H |
| B-II-97 | —OCF3 | —F | —H | —CN | —F | —H |
| B-II-98 | —F | —OCF₃ | —H | —CN | —F | —H |
| B-II-99 | —OCH₃ | —H | —H | —CN | —H | —F |
| B-II-100 | —OCF₃ | —H | —H | —CN | —H | —F |
| B-II-101 | —CF₃ | —H | —H | —CN | —H | —F |
| B-II-102 | —CN | —H | —H | —CN | —H | —F |
| B-II-103 | —F | —H | —H | —CN | —H | —F |
| B-II-104 | —CF₂H | —H | —H | —CN | —H | —F |
| B-II-105 | —SO₂CH₃ | —H | —H | —CN | —H | —F |
| B-II-106 | —F | —F | —H | —CN | —H | —F |
| B-II-107 | —CN | —F | —H | —CN | —H | —F |
| B-II-108 | —F | —CN | —H | —CN | —H | —F |
| B-II-109 | —CF₃ | —F | —H | —CN | —H | —F |
| B-II-110 | —F | —CF₃ | —H | —CN | —H | —F |
| B-II-111 | —OCF₃ | —F | —H | —CN | —H | —F |
| B-II-112 | —F | —OCF₃ | —H | —CN | —H | —F |
| B-II-113 | —OCH₃ | —H | —F | —H | —H | —H |
| B-II-114 | —OCF₃ | —H | —F | —H | —H | —H |
| B-II-115 | —CF₃ | —H | —F | —H | —H | —H |
| B-II-116 | —CN | —H | —F | —H | —H | —H |
| B-II-117 | —F | —H | —F | —H | —H | —H |
| B-II-118 | —CF₂H | —H | —F | —H | —H | —H |
| B-II-119 | —SO₂CH₃ | —H | —F | —H | —H | —H |
| B-II-120 | —F | —F | —F | —H | —H | —H |
| B-II-121 | —CN | —F | —F | —H | —H | —H |
| B-II-122 | —F | —CN | —F | —H | —H | —H |
| B-II-123 | —CF₃ | —F | —F | —H | —H | —H |
| B-II-124 | —F | —CF₃ | —F | —H | —H | —H |
| B-II-125 | —OCF₃ | —F | —F | —H | —H | —H |
| B-II-126 | —F | —OCF₃ | —F | —H | —H | —H |
| B-II-127 | —OCH₃ | —H | —H | —F | —H | —H |
| B-II-128 | —OCF₃ | —H | —H | —F | —H | —H |
| B-II-129 | —CF₃ | —H | —H | —F | —H | —H |
| B-II-130 | —CN | —H | —H | —F | —H | —H |
| B-II-131 | —F | —H | —H | —F | —H | —H |
| B-II-132 | —CF₂H | —H | —H | —F | —H | —H |
| B-II-133 | —SO₂CH₃ | —H | —H | —F | —H | —H |
| B-II-134 | —F | —F | —H | —F | —H | —H |
| B-II-135 | —CN | —F | —H | —F | —H | —H |
| B-II-136 | —F | —CN | —H | —F | —H | —H |
| B-II-137 | —CF₃ | —F | —H | —F | —H | —H |
| B-II-138 | —F | —CF₃ | —H | —F | —H | —H |
| B-II-139 | —OCF₃ | —F | —H | —F | —H | —H |
| B-II-140 | —F | —OCF₃ | —H | —F | —H | —H |
| B-II-141 | —OCH₃ | —H | —F | —F | —H | —H |
| B-II-142 | —OCF₃ | —H | —F | —F | —H | —H |
| B-II-143 | —CF₃ | —H | —F | —F | —H | —H |
| B-II-144 | —CN | —H | —F | —F | —H | —H |
| B-II-145 | —F | —H | —F | —F | —H | —H |
| B-II-146 | —CF₂H | —H | —F | —F | —H | —H |
| B-II-147 | —SO₂CH₃ | —H | —F | —F | —H | —H |
| B-II-148 | —F | —F | —F | —F | —H | —H |
| B-II-149 | —CN | —F | —F | —F | —H | —H |
| B-II-150 | —F | —CN | —F | —F | —H | —H |
| B-II-151 | —CF₃ | —F | —F | —F | —H | —H |
| B-II-152 | —F | —CF₃ | —F | —F | —H | —H |
| B-II-153 | —OCF₃ | —F | —F | —F | —H | —H |
| B-II-154 | —F | —OCF₃ | —F | —F | —H | —H |
| B-II-155 | —OCH₃ | —H | —F | —H | —F | —H |
| B-II-156 | —OCF₃ | —H | —F | —H | —F | —H |
| B-II-157 | —CF₃ | —H | —F | —H | —F | —H |
| B-II-158 | —CN | —H | —F | —H | —F | —H |
| B-II-159 | —F | —H | —F | —H | —F | —H |
| B-II-160 | —CF₂H | —H | —F | —H | —F | —H |
| B-II-161 | —SO₂CH₃ | —H | —F | —H | —F | —H |
| B-II-162 | —F | —F | —F | —H | —F | —H |
| B-II-163 | —CN | —F | —F | —H | —F | —H |
| B-II-164 | —F | —CN | —F | —H | —F | —H |
| B-II-165 | —CF₃ | —F | —F | —H | —F | —H |
| B-II-166 | —F | —CF₃ | —F | —H | —F | —H |
| B-II-167 | —OCF₃ | —F | —F | —H | —F | —H |
| B-II-168 | —F | —OCF₃ | —F | —H | —F | —H |
| B-II-169 | —OCH₃ | —H | —F | —H | —H | —F |
| B-II-170 | —OCF₃ | —H | —F | —H | —H | —F |
| B-II-171 | —CF₃ | —H | —F | —H | —H | —F |
| B-II-172 | —CN | —H | —F | —H | —H | —F |
| B-II-173 | —F | —H | —F | —H | —H | —F |
| B-II-174 | —CF₂H | —H | —F | —H | —H | —F |
| B-II-175 | —SO₂CH₃ | —H | —F | —H | —H | —F |
| B-II-176 | —F | —F | —F | —H | —H | —F |
| B-II-177 | —CN | —F | —F | —H | —H | —F |
| B-II-178 | —F | —CN | —F | —H | —H | —F |
| B-II-179 | —CF₃ | —F | —F | —H | —H | —F |
| B-II-180 | —F | —CF | —F | —H | —H | —F |
| B-II-181 | —OCF₃ | —F | —F | —H | —H | —F |
| B-II-182 | —F | —OCF₃ | —F | —H | —H | —F |
| B-II-183 | —OCH₃ | —H | —H | —F | —F | —H |
| B-II-184 | —OCF₃ | —H | —H | —F | —F | —H |
| B-II-185 | —CF₃ | —H | —H | —F | —F | —H |
| B-II-186 | —CN | —H | —H | —F | —F | —H |
| B-II-187 | —F | —H | —H | —F | —F | —H |
| B-II-188 | —CF₂H | —H | —H | —F | —F | —H |
| B-II-189 | —SO₂CH₃ | —H | —H | —F | —F | —H |
| B-II-190 | —F | —F | —H | —F | —F | —H |
| B-II-191 | —CN | —F | —H | —F | —F | —H |
| B-II-192 | —F | —CN | —H | —F | —F | —H |
| B-II-193 | —CF₃ | —F | —H | —F | —F | —H |
| B-II-194 | —F | —CF₃ | —H | —F | —F | —H |
| B-II-195 | —OCF₃ | —F | —H | —F | —F | —H |
| B-II-196 | —F | —OCF₃ | —H | —F | —F | —H |
| B-II-197 | —OCH₃ | —H | —CF₃ | —H | —H | —H |
| B-II-198 | —OCF₃ | —H | —CF₃ | —H | —H | —H |
| B-II-199 | —CF₃ | —H | —CF₃ | —H | —H | —H |
| B-II-200 | —CN | —H | —CF₃ | —H | —H | —H |
| B-II-201 | —F | —H | —CF₃ | —H | —H | —H |
| B-II-202 | —CF₂H | —H | —CF₃ | —H | —H | —H |
| B-II-203 | —SO₂CH₃ | —H | —CF₃ | —H | —H | —H |
| B-II-204 | —F | —F | —CF₃ | —H | —H | —H |
| B-II-205 | —CN | —F | —CF₃ | —H | —H | —H |
| B-II-206 | —F | —CN | —CF₃ | —H | —H | —H |
| B-II-207 | —CF₃ | —F | —CF₃ | —H | —H | —H |
| B-II-208 | —F | —CF₃ | —CF₃ | —H | —H | —H |
| B-II-209 | —OCF₃ | —F | —CF₃ | —H | —H | —H |
| B-II-210 | —F | —OCF₃ | —CF₃ | —H | —H | —H |
| B-II-211 | —OCH₃ | —H | —CF₃ | —F | —H | —H |
| B-II-212 | —OCF₃ | —H | —CF₃ | —F | —H | —H |
| B-II-213 | —CF₃ | —H | —CF₃ | —F | —H | —H |
| B-II-214 | —CN | —H | —CF₃ | —F | —H | —H |
| B-II-215 | —F | —H | —CF₃ | —F | —H | —H |
| B-II-216 | —CF₂H | —H | —CF₃ | —F | —H | —H |
| B-II-217 | —SO₂CH₃ | —H | —CF₃ | —F | —H | —H |
| B-II-218 | —F | —F | —CF₃ | —F | —H | —H |
| B-II-219 | —CN | —F | —CF₃ | —F | —H | —H |
| B-II-220 | —F | —CN | —CF₃ | —F | —H | —H |
| B-II-221 | —CF₃ | —F | —CF₃ | —F | —H | —H |
| B-II-222 | —F | —CF₃ | —CF₃ | —F | —H | —H |
| B-II-223 | —OCF₃ | —F | —CF₃ | —F | —H | —H |
| B-II-224 | —F | —OCF₃ | —CF₃ | —F | —H | —H |
| B-II-225 | —OCH₃ | —H | —CF₃ | —H | —F | —H |
| B-II-226 | —OCF₃ | —H | —CF₃ | —H | —F | —H |
| B-II-227 | —CF₃ | —H | —CF₃ | —H | —F | —H |
| B-II-228 | —CN | —H | —CF₃ | —H | —F | —H |
| B-II-229 | —F | —H | —CF₃ | —H | —F | —H |
| B-II-230 | —CF₂H | —H | —CF₃ | —H | —F | —H |
| B-II-231 | —SO₂CH₃ | —H | —CF₃ | —H | —F | —H |
| B-II-232 | —F | —F | —CF₃ | —H | —F | —H |
| B-II-233 | —CN | —F | —CF₃ | —H | —F | —H |
| B-II-234 | —F | —CN | —CF₃ | —H | —F | —H |
| B-II-235 | —CF₃ | —F | —CF₃ | —H | —F | —H |
| B-II-236 | —F | —CF₃ | —CF₃ | —H | —F | —H |
| B-II-237 | —OCF₃ | —F | —CF₃ | —H | —F | —H |
| B-II-238 | —F | —OCF₃ | —CF₃ | —H | —F | —H |
| B-II-239 | —OCH₃ | —H | —CF₃ | —H | —H | —F |
| B-II-240 | —OCF₃ | —H | —CF₃ | —H | —H | —F |
| B-II-241 | —CF₃ | —H | —CF₃ | —H | —H | —F |
| B-II-242 | —CN | —H | —CF₃ | —H | —H | —F |
| B-II-243 | —F | —H | —CF₃ | —H | —H | —F |
| B-II-244 | —CF₂H | —H | —CF₃ | —H | —H | —F |
| B-II-245 | —SO₂CH₃ | —H | —CF₃ | —H | —H | —F |
| B-II-246 | —F | —F | —CF₃ | —H | —H | —F |
| B-II-247 | —CN | —F | —CF₃ | —H | —H | —F |
| B-II-248 | —F | —CN | —CF₃ | —H | —H | —F |
| B-II-249 | —CF₃ | —F | —CF₃ | —H | —H | —F |
| B-II-250 | —F | —CF₃ | —CF₃ | —H | —H | —F |
| B-II-251 | —OCF₃ | —F | —CF₃ | —H | —H | —F |
| B-II-252 | —F | —OCF₃ | —CF₃ | —H | —H | —F |
| B-II-253 | —OCH₃ | —H | —F | —CF₃ | —H | —H |
| B-II-254 | —OCF₃ | —H | —F | —CF₃ | —H | —H |
| B-II-255 | —CF₃ | —H | —F | —CF₃ | —H | —H |
| B-II-256 | —CN | —H | —F | —CF₃ | —H | —H |
| B-II-257 | —F | —H | —F | —CF₃ | —H | —H |
| B-II-258 | —CF₂H | —H | —F | —CF₃ | —H | —H |
| B-II-259 | —SO₂CH₃ | —H | —F | —CF₃ | —H | —H |
| B-II-260 | —F | —F | —F | —CF₃ | —H | —H |
| B-II-261 | —CN | —F | —F | —CF₃ | —H | —H |
| B-II-262 | —F | —CN | —F | —CF₃ | —H | —H |
| B-II-263 | —CF₃ | —F | —F | —CF₃ | —H | —H |
| B-II-264 | —F | —CF₃ | —F | —CF₃ | —H | —H |
| B-II-265 | —OCF₃ | —F | —F | —CF₃ | —H | —H |
| B-II-266 | —F | —OCF₃ | —F | —CF₃ | —H | —H |
| B-II-267 | —OCH₃ | —H | —H | —CF₃ | —F | —H |
| B-II-268 | —OCF₃ | —H | —H | —CF₃ | —F | —H |
| B-II-269 | —CF₃ | —H | —H | —CF₃ | —F | —H |
| B-II-270 | —CN | —H | —H | —CF₃ | —F | —H |
| B-II-271 | —F | —H | —H | —CF₃ | —F | —H |
| B-II-272 | —CF₂H | —H | —H | —CF₃ | —F | —H |
| B-II-273 | —SO₂CH₃ | —H | —H | —CF₃ | —F | —H |
| B-II-274 | —F | —F | —H | —CF₃ | —F | —H |
| B-II-275 | —CN | —F | —H | —CF₃ | —F | —H |
| B-II-276 | —F | —CN | —H | —CF₃ | —F | —H |
| B-II-277 | —CF₃ | —F | —H | —CF₃ | —F | —H |
| B-II-278 | —F | —CF₃ | —H | —CF₃ | —F | —H |
| B-II-279 | —OCF₃ | —F | —H | —CF₃ | —F | —H |
| B-II-280 | —F | —OCF₃ | —H | —CF₃ | —F | —H |
| B-II-281 | —OCH₃ | —H | —H | —CF₃ | —H | —F |
| B-II-282 | —OCF₃ | —H | —H | —CF₃ | —H | —F |
| B-II-283 | —CF₃ | —H | —H | —CF₃ | —H | —F |
| B-II-284 | —CN | —H | —H | —CF₃ | —H | —F |
| B-II-285 | —F | —H | —H | —CF₃ | —H | —F |
| B-II-286 | —CF₂H | —H | —H | —CF₃ | —H | —F |
| B-II-287 | —SO₂CH₃ | —H | —H | —CF₃ | —H | —F |
| B-II-288 | —F | —F | —H | —CF₃ | —H | —F |
| B-II-289 | —CN | —F | —H | —CF₃ | —H | —F |
| B-II-290 | —F | —CN | —H | —CF₃ | —H | —F |
| B-II-291 | —CF₃ | —F | —H | —CF₃ | —H | —F |
| B-II-292 | —F | —CF₃ | —H | —CF₃ | —H | —F |
| B-II-293 | —OCF₃ | —F | —H | —CF₃ | —H | —F |
| B-II-294 | —F | —OCF₃ | —H | —CF₃ | —H | —F |

37. A compound, or a tautomer, stereoisomer, mixture of stereoisomers, isotopically enriched analog, or pharmaceutically acceptable salt thereof, represented by Formula B-HA, as in Table B-5: 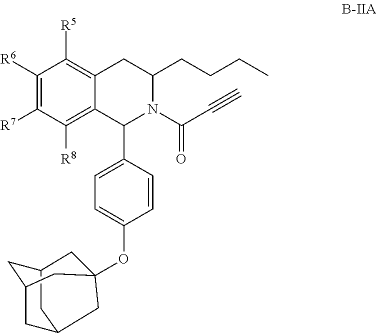
TABLE B-5

| Compound No. | R⁵ | R⁶ | R⁷ | R⁸ |
|---|---|---|---|---|
| B-IIA-1 | —OCH₃ | —H | —H | —H |
| B-IIA-2 | —H | —OCH₃ | —H | —H |
| B-IIA-3 | —H | —H | —OCH₃ | —H |
| B-IIA-4 | —H | —H | —H | —OCH₃ |
| B-IIA-5 | —OCF₃ | —H | —H | —H |
| B-IIA-6 | —H | —OCF₃ | —H | —H |
| B-IIA-7 | —H | —H | —OCF₃ | —H |
| B-IIA-8 | —H | —H | —H | —OCF₃ |
| B-IIA-9 | —CF₃ | —H | —H | —H |
| B-IIA-10 | —H | —CF₃ | —H | —H |
| B-IIA-11 | —H | —H | —CF₃ | —H |
| B-IIA-12 | —H | —H | —H | —CF₃ |
| B-IIA-13 | —CN | —H | —H | —H |
| B-IIA-14 | —H | —CN | —H | —H |
| B-IIA-15 | —H | —H | —CN | —H |
| B-IIA-16 | —H | —H | —H | —CN |
| B-IIA-17 | —F | —H | —H | —H |
| B-IIA-18 | —H | —F | —H | —H |
| B-IIA-19 | —H | —H | —F | —H |
| B-IIA-20 | —H | —H | —H | —F |
| B-IIA-21 | —CF₂H | —H | —H | —H |
| B-IIA-22 | —H | —CF₂H | —H | —H |
| B-IIA-23 | —H | —H | —CF₂H | —H |
| B-IIA-24 | —H | —H | —H | —CF₂H |
| B-IIA-25 | —SO₂CH₃ | —H | —H | —H |
| B-IIA-26 | —H | —SO₂CH₃ | —H | —H |
| B-IIA-27 | —H | —H | —SO₂CH₃ | —H |
| B-IIA-28 | —H | —H | —H | —SO₂CH₃ |
| B-IIA-29 | —SO₂NHCH₃ | —H | —H | —H |
| B-IIA-30 | —H | —SO₂NHCH₃ | —H | —H |
| B-IIA-31 | —H | —H | —SO₂NHCH₃ | —H |
| B-IIA-32 | —H | —H | —H | —SO₂NHCH₃ |

38. A compound, or a tautomer, stereoisomer, mixture of stereoisomers, isotopically enriched analog, or pharmaceutically acceptable salt thereof, represented by Formula B-IIB, as in Table B-6: 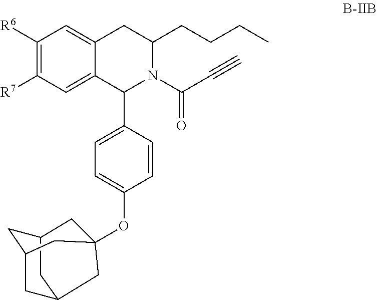
TABLE B-6

| Compound No. | R⁶ | R⁷ |
|---|---|---|
| B-IIB-1 | —OCH₃ | —F |
| B-IIB-2 | —F | —OCH₃ |
| B-IIB-3 | —OCF₃ | —F |
| B-IIB-4 | —F | —OCF₃ |
| B-IIB-5 | —F | —F |
| B-IIB-6 | —OCH₃ | —CN |
| B-IIB-7 | —CN | —OCH₃ |
| B-IIB-8 | —SO₂CH₃ | —F |
| B-IIB-9 | —F | —SO₂CH₃ |
| B-IIB-10 | —CN | —F |
| B-IIB-11 | —F | —CN |

39. A compound, or a tautomer, stereoisomer, mixture of stereoisomers, isotopically enriched analog, or pharmaceutically acceptable salt thereof, as in Table B-7.

40. A compound, or a tautomer, stereoisomer, mixture of stereoisomers, isotopically enriched analog, or pharmaceutically acceptable salt thereof, selected from:

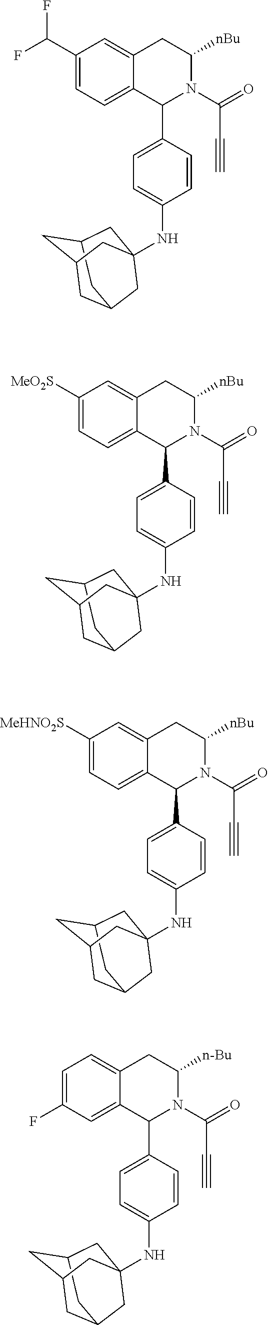

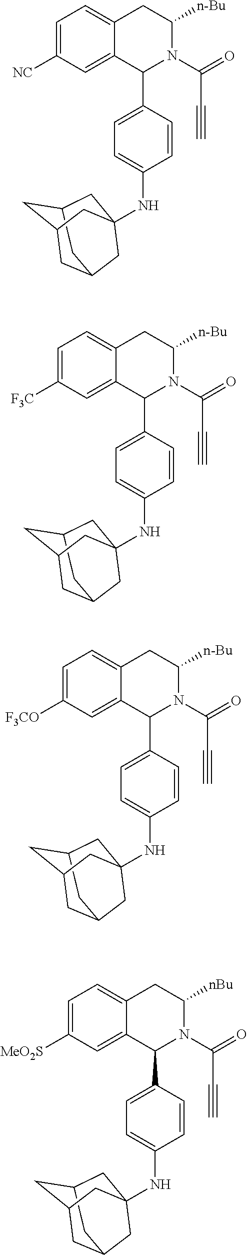

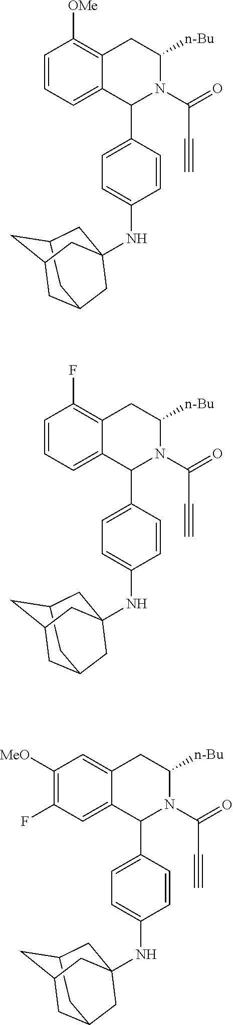

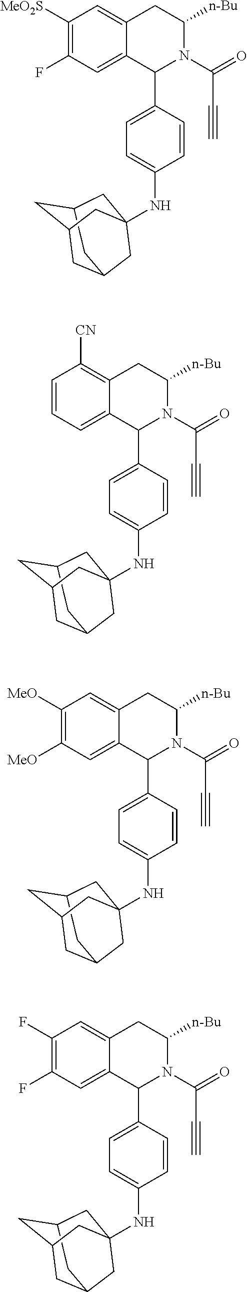

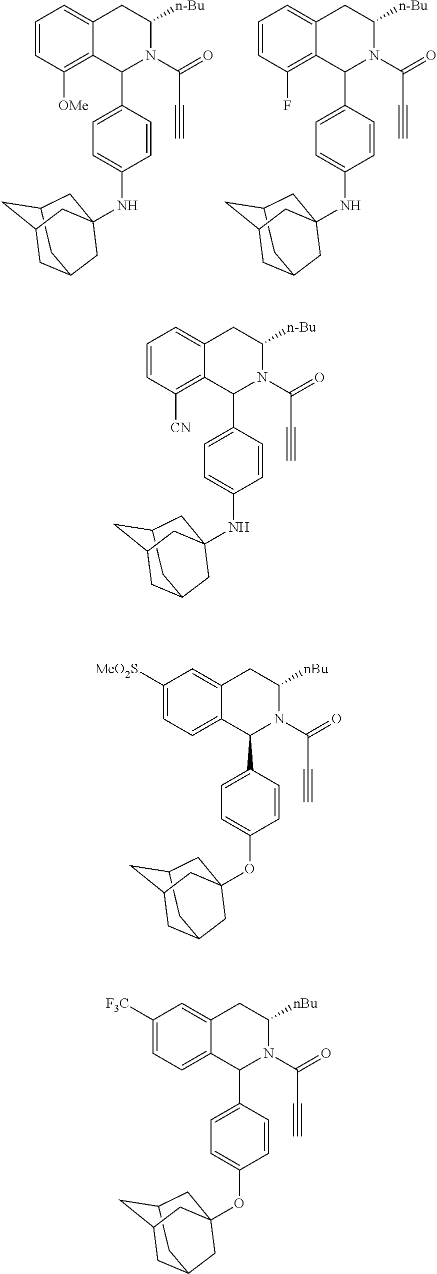

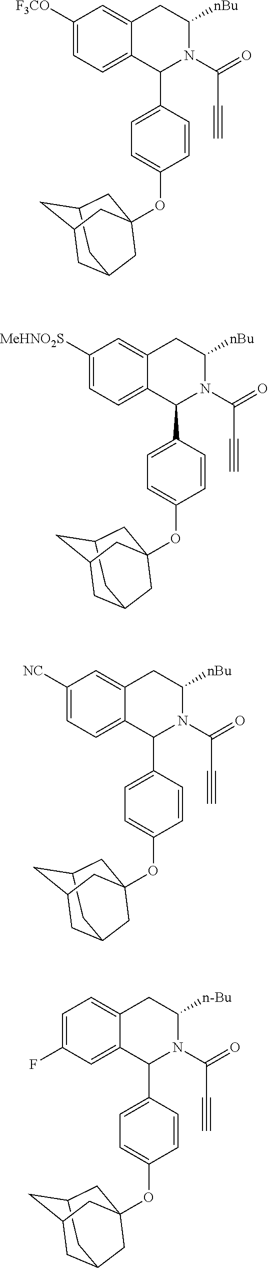

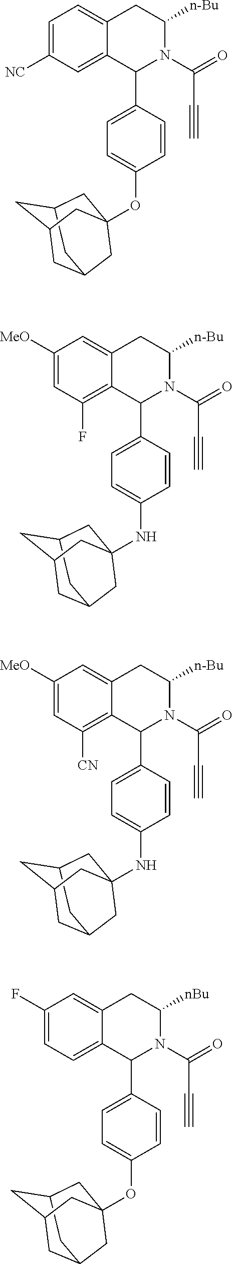

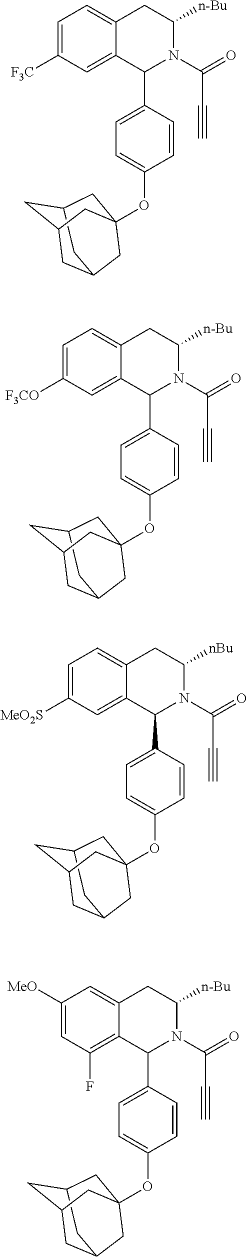

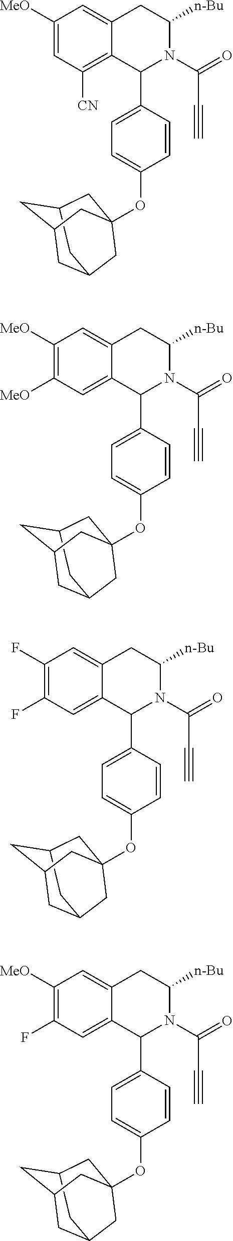

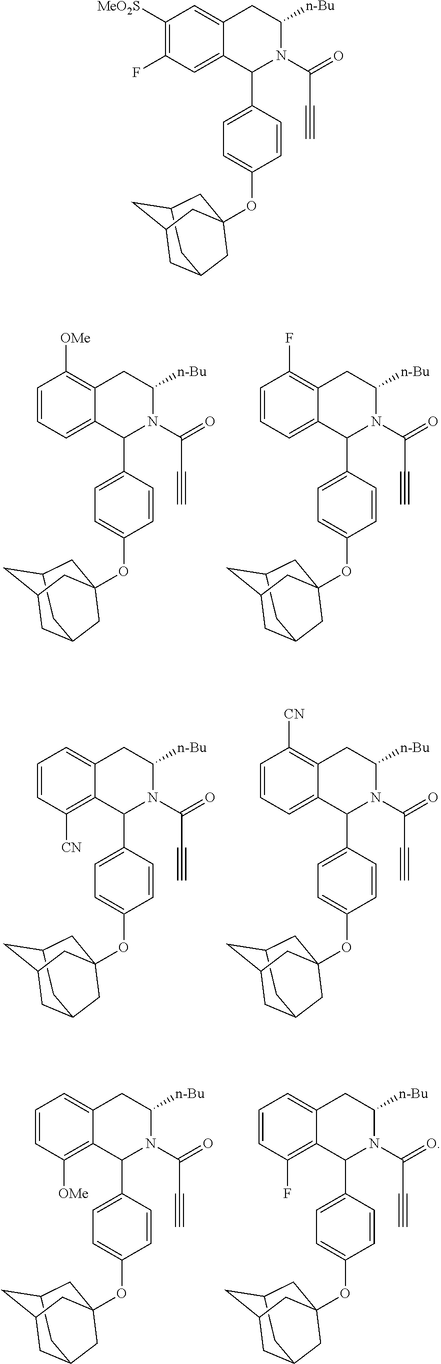

41. A compound, or a tautomer, stereoisomer, mixture of stereoisomers, isotopically enriched analog, or pharmaceutically acceptable salt thereof, selected from:

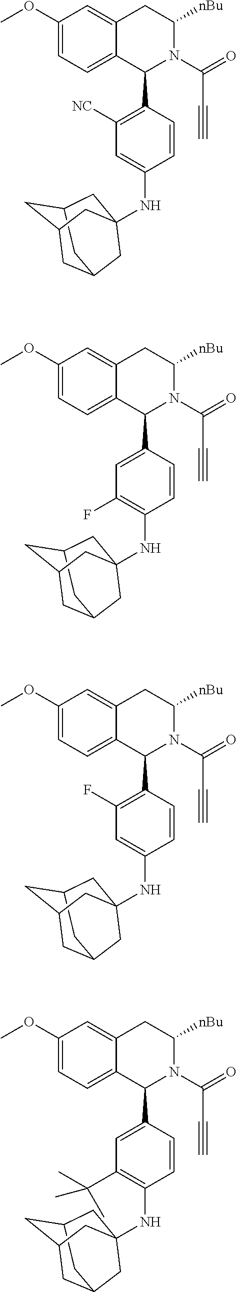

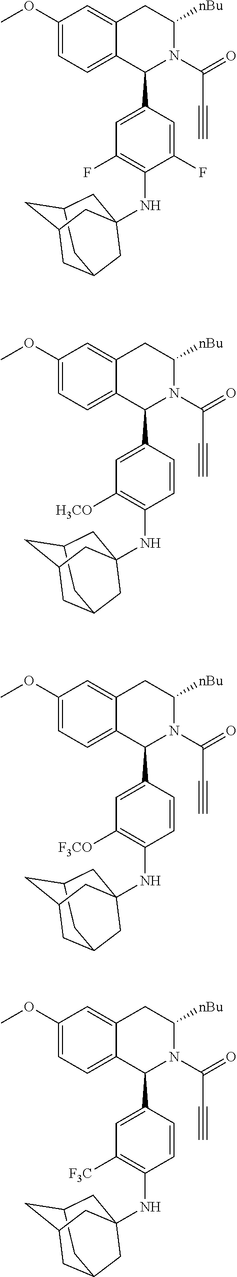

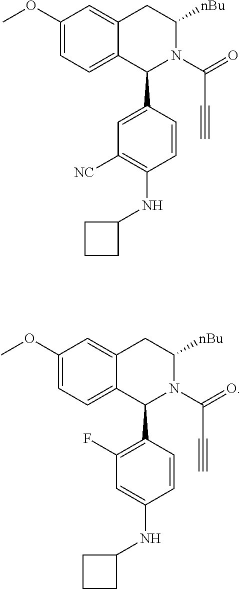

42. A compound, or a tautomer, stereoisomer, mixture of stereoisomers, isotopically enriched analog, or pharmaceutically acceptable salt thereof, as in Table B-10.

43. A compound of Formula B-X, or a tautomer, stereoisomer, mixture of stereoisomers, isotopically enriched analog, or pharmaceutically acceptable salt thereof: 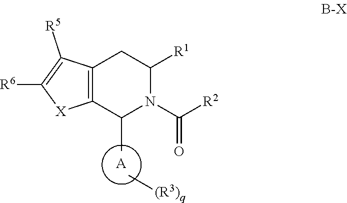
wherein:
ring A is C₄-C₁₀ cycloalkyl, heterocyclyl, aryl, or heteroaryl;
X is —CR¹⁴═CR¹⁴— or —CR¹⁴═N—;
q is 0, 1, 2, or 3;
R¹ is C₁-C₆ alkyl, C₂-C₆ alkenyl, C₂-C₆alkynyl, C₁-C₆ haloalkyl, C₃-C₁₀ cycloalkyl, —CN, —OR²⁷, —C(O)OR²⁶, —C(O)N(R²⁷)₂—OC(O)R²⁶, —S(O)₂R²⁸, —S(O)₂N(R²⁷)₂, —S(O)N(R²⁷)₂, —S(O)R²⁸, —N(R²⁷)₂, —NO₂, —C₁-C₆ alkyl-OR²⁷, or —Si(R¹⁵)₃;
R² is 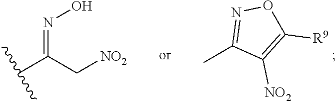
each R³ is independently halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹²)₃, —SF₅, —C(O)OR²⁶, —C(O)N(R²⁷)₂, —NR¹²C(O)R²⁸, —NR¹²C(O)OR²⁸, —OC(O)N(R²⁷)₂, —OC(O)R²⁸, —C(O)R²⁶, —OC(O)CHR²⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁵ is hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R is independently unsubstituted or substituted with one to three R¹⁰; and
R⁶ is hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁶ is independently unsubstituted or substituted with one to three R¹⁰.
R⁹ is C₁-C₄ alkyl, C₁-C₄ haloalkyl, or C₂-C₄ alkenyl, wherein each C₁-C₄ alkyl, C₁-C₄ haloalkyl, or C₂-C₄ alkenyl of R⁹ is independently unsubstituted or substituted with one to three R¹¹;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl;
each R¹² is independently hydrogen, C₁-C₆ alkyl, C₁-C₆ alkylheterocyclyl, or C₃-C₁₀ cycloalkyl;
each R¹³ is independently C₁-C₆ alkyl or C₃-C₁₀ cycloalkyl;
each R¹⁴ is independently hydrogen, halo, —CN, —OH, —OR²⁸, —NH₂, —NHR²⁸, —N(R²⁸)₂, —S(O)₂R²⁸, —S(O)R²⁸, —S(O)₂N(R²⁷)₂, —S(O)N(R²⁷)₂, —NO₂, —Si(R¹⁵)₃, —C(O)OR²⁶, —C(O)N(R²⁷)₂, —NR¹²C(O)R²⁸, —OC(O)R²⁸, —C(O)R²⁶, —NR¹²C(O)OR²⁸, —OC(O)N(R²⁷)₂, —OC(O)CHR²⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkylaryl, —C₂-C₆ alkenylaryl, —C₁-C₆ alkylheteroaryl, or —C₂-C₆ alkenylheteroaryl;
each R²⁶ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl; wherein each R²⁶ is independently further substituted with one to three R¹¹;
each R²⁷ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R²⁷, together with the nitrogen atom to which they are attached, form a 4 to 7 membered heterocyclyl, wherein each R²⁷ or ring formed thereby is independently further substituted with one to three R¹¹;
each R²⁸ is independently —(CH₂)ᵤP(O)R^{a}R^{b}, —CH₂)ᵤCH₂OP(O)(R^{a})(R), C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R²⁸ is independently further substituted with one to three R¹¹;
u is 0, 1, 2, 3, or 4;
each R^{a} is independently selected from the group consisting of hydrogen, —OR¹², —N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently further substituted with one to three R¹¹; and
each R^{b} is independently selected from the group consisting of hydrogen, —OR¹², —N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R^{a} is independently further substituted with one to three R¹¹; or
R^{a} and R^{b} combine together to form a ring consisting of 3-8 ring atoms that are C, N, O, or S;
wherein the ring is unsubstituted or is substituted with one to three R¹¹.

44. The compound of claim 43, wherein R¹ is C₁-C₆ alkyl, C₂-C₆ alkenyl, C₂-C₆alkynyl, C₁-C₆ haloalkyl, C₃-C₁₀ cycloalkyl, —CN, —C(O)OR²⁶, —C(O)N(R²⁷)₂, —N(R²⁷)₂, —OR²⁷, or —C₁-C₆ alkyl-OR²⁷.

45. The compound of claim 44, or a tautomer, stereoisomer, mixture of stereoisomers, isotopically enriched analog, or pharmaceutically acceptable salt thereof, wherein R¹ is C₁-C₆ alkyl, C₃-C₁₀ cycloalkyl, or —C₁-C₆ alkyl-OR²⁷.

46. The compound of claim 45, or a tautomer, stereoisomer, mixture of stereoisomers, isotopically enriched analog, or pharmaceutically acceptable salt thereof, wherein R¹ is C₁-C₄ alkyl.

47. The compound of any one of claims 43-46, or a tautomer, stereoisomer, mixture of stereoisomers, isotopically enriched analog, or pharmaceutically acceptable salt thereof, wherein R⁹ is C₁-C₄ alkyl.

48. The compound of any one of claims 43-47, or a tautomer, stereoisomer, mixture of stereoisomers, isotopically enriched analog, or pharmaceutically acceptable salt thereof, wherein R² is:

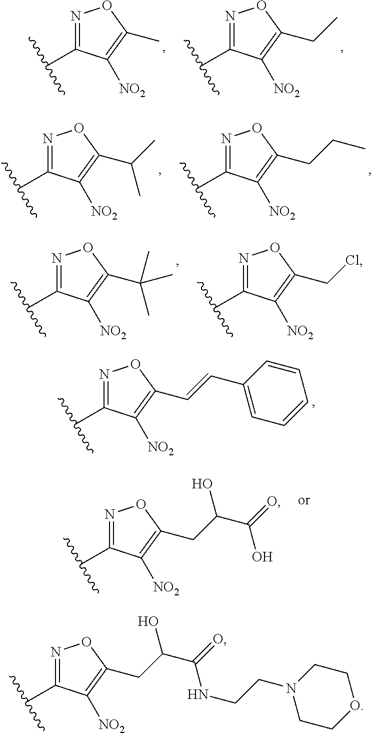

49. The compound of any one of claims 43-48, or a tautomer, stereoisomer, mixture of stereoisomers, isotopically enriched analog, or pharmaceutically acceptable salt thereof, wherein R³ is C₃-C₁₀ cycloalkyl.

50. The compound of any one of claims 43-49, or a tautomer, stereoisomer, mixture of stereoisomers, isotopically enriched analog, or pharmaceutically acceptable salt thereof, wherein R⁶ is halo, —CN, —OR²⁸, —S(O)₂R²⁸, —S(O)₂N(R²⁷)₂, or C₁-C₆ alkyl, wherein each C₁-C₆ alkyl of R⁶ is independently unsubstituted or substituted with one to three R¹⁰.

51. The compound of any one of claims 43-50, or a tautomer, stereoisomer, mixture of stereoisomers, isotopically enriched analog, or pharmaceutically acceptable salt thereof, wherein R⁶ is —CN, —F, CF₂H, —OCH₃, —OCF₃, —CF₃, —S(O)₂CH₃, or —S(O)₂ NHCH₃.

52. The compound of any one of claims 43-51, or a tautomer, stereoisomer, mixture of stereoisomers, isotopically enriched analog, or pharmaceutically acceptable salt thereof, wherein R⁵ is hydrogen.

53. The compound of any one of claims 43-52, or a tautomer, stereoisomer, mixture of stereoisomers, isotopically enriched analog, or pharmaceutically acceptable salt thereof, wherein one R¹⁴ is hydrogen and the other R¹⁴ is hydrogen, halo, —CN, —OR²⁸, —S(O)₂R²⁸, —S(O)₂N(R²⁷)₂, or C₁-C₆ alkyl, wherein each C₁-C₆ alkyl of R¹⁴ is independently unsubstituted or substituted with one to three R¹⁰.

54. A pharmaceutical composition comprising a compound, or a tautomer, stereoisomer, mixture of stereoisomers, isotopically enriched analog, or pharmaceutically acceptable salt thereof, of any one of claims 1-53, and a pharmaceutically acceptable carrier.

55. A pharmaceutical composition comprising a compound, or a tautomer, stereoisomer, mixture of stereoisomers, isotopically enriched analog, or pharmaceutically acceptable salt thereof, of Table A-1A, Table A-2A, Table A-1B, Table A-1C, Table B-1, Table B-2, Table B-3, Table B-4, Table B-5, Table B-6, Table B-7, Table B-8, Table B-9, Table B-10, Table B-XIA, or Table B-XIB, and a pharmaceutically acceptable carrier.

56. A method of inhibiting GPX4 in a cell, comprising contacting a cell with an effective amount of a compound of any one of claims 1-53, the pharmaceutical composition of claim 54 or 55, or a compound of formula A-I: 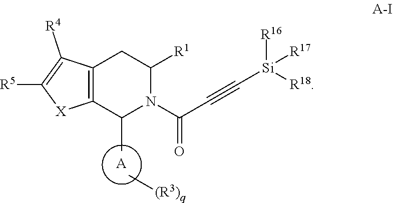
or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R⁵, together with the atoms to which they are attached, can form a 6-membered aryl or 6-membered heteroaryl, wherein each aryl or heteroaryl is unsubstituted or substituted with one to three R¹⁴;
each R⁶ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently further substituted with one to three R¹¹;
each R⁷ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷, together with the nitrogen atom to which they are attached, form a 4 to 7 membered heterocyclyl, wherein each R or ring formed thereby is independently further substituted with one to three R¹¹;
each R⁸ is independently C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently further substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

57. The method of claim 56, wherein the cell is a cancer cell.

58. A method of treating cancer in a subject, comprising administering to a subject having cancer a therapeutically effective amount of a compound of any one of claims 1-53, the pharmaceutical composition of claim 54 or 55, or a compound of formula A-I: 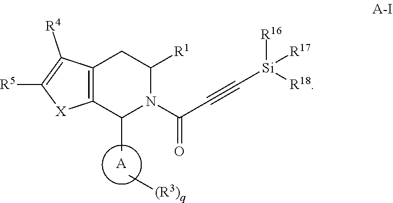
or a tautomer, stereoisomer, mixture of stereoisomers, isotopically enriched analog, or pharmaceutically acceptable salt thereof, wherein:
X is —NR²²—, —O—, —S—, —N═CR⁹—, —CR⁹═CR⁹—, or —CR⁹═N—;
ring A is C₄-C₁₀ cycloalkyl, heterocyclyl, aryl, or heteroaryl;
q is 0, 1, 2, or 3;
each R¹ is independently C₁-C₆ alkyl, C₂-C₆ alkenyl, C₂-C₆alkynyl, C₁-C₆ haloalkyl, C₃-C₁₀ cycloalkyl, —CN, —OH, —C(O)OR⁶, —C(O)N(R⁷)₂—OC(O)R⁶, —S(O)₂R, —S(O)₂N(R⁷)₂, —S(O)N(R⁷)₂, —S(O)R⁸, —NH₂, —NHR⁸, —N(R⁸)₂, —NO₂, —OR⁸, —C₁-C₆ alkyl-OH, —C₁-C₆ alkyl-OR⁸, —C₁-C₆ alkyl-C₃-C₁₀ cycloalkyl, or —Si(R¹⁵)₃;
R²² is hydrogen or C₁-C₆ alkyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R³ is independently unsubstituted or substituted with one to three R¹⁰;
R⁴ and R⁵ are each independently hydrogen, halo, —CN, —OH, —OR⁸, —NH₂, —NHR⁸, —N(R⁸)₂, —S(O)₂R, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁴ and R⁵ is independently unsubstituted or substituted with one to three R¹⁰; or
when X is —NR²²—, —O—, or —S—, then R⁴ and R⁵, together with the atoms to which they are attached, can form a 6-membered aryl or 6-membered heteroaryl, wherein each aryl or heteroaryl is unsubstituted or is substituted with one to three R¹⁴;
each R⁶ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each R⁶ is independently further substituted with one to three R¹¹;
each R⁷ is independently hydrogen, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₆ cycloalkyl, —C₂-C₆ alkenylC₃-C₆ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, —C₂-C₆ alkenylheteroaryl, or two R⁷ together with the nitrogen atom to which they are attached, form a 4 to 7 membered heterocyclyl, wherein each R or ring formed thereby is independently further substituted with one to three R¹¹;
each R⁸ is independently C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, —C₁-C₆ alkylheteroaryl, or —C₂-C₆ alkenylheteroaryl, wherein each R⁸ is independently further substituted with one to three R¹¹;
each R⁹ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, —C₁-C₆ alkylC₃-C₁₀ cycloalkyl, —C₂-C₆ alkenylC₃-C₁₀ cycloalkyl, —C₁-C₆ alkylheterocyclyl, —C₂-C₆ alkenylheterocyclyl, —C₁-C₆ alkylaryl, —C₂-C₆ alkenylaryl, C₁-C₆ alkylheteroaryl, or —C₂-C₆ alkenylheteroaryl of R⁹ is independently unsubstituted or substituted with one to three R¹⁰;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl, wherein each C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆ alkyl, C₁-C₆ haloalkyl, C₂-C₆ alkenyl, C₂-C₆alkynyl, C₃-C₁₀ cycloalkyl, heterocyclyl, aryl, or heteroaryl;
each R¹² is independently hydrogen, C₁-C₆ alkyl, or C₃-C₁₀ cycloalkyl;
each R¹³ is independently C₁-C₆ alkyl or C₃-C₁₀ cycloalkyl;
each R¹⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆ alkyl, C₂-C₆ alkenyl, C₂-C₆alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or C₂-C₆ alkenylheteroaryl, wherein each C₁-C₆ alkyl, C₂-C₆ alkenyl, C₂-C₆alkynyl, C₃-C₁₀ cycloalkyl, heterocyclyl, aryl, heteroaryl, C₁-C₆ alkylC₃-C₁₀ cycloalkyl, C₂-C₆ alkenylC₃-C₁₀ cycloalkyl, C₁-C₆ alkylheterocyclyl, C₂-C₆ alkenylheterocyclyl, C₁-C₆ alkylaryl, C₂-C₆ alkenylaryl, C₁-C₆ alkylheteroaryl, or —C₂-C₆ alkenylheteroaryl of R¹⁴ is independently unsubstituted or substituted with one to three R¹⁰;
each R¹⁵ is independently C₁-C₆ alkyl, C₂-C₆ alkenyl, aryl, heteroaryl, —C₁-C₆ alkyl-aryl, —C₂-C₆ alkenyl-aryl, —C₁-C₆ alkyl-heteroaryl, or —C₂-C₆ alkenyl-heteroaryl;
R¹⁶ is C₁-C₆ alkyl that is unsubstituted or is substituted with one to three R¹⁰;
R¹⁷ is hydrogen or C₁-C₆ alkyl that is unsubstituted or is substituted with one to three R¹⁰; and
R¹⁸ is hydrogen, C₁-C₆ alkyl, or —OC₁-C₆ alkyl, wherein each C₁-C₆ alkyl or —OC₁-C₆ alkyl of R¹⁸ is unsubstituted or is substituted with one to three R¹⁰.

59. The method of claim 58, wherein the cancer is adrenocortical cancer, anal cancer, biliary cancer, bladder cancer, bone cancer, brain cancer, breast cancer, cervical cancer, colon cancer, endometrial cancer, esophageal cancer, head and neck cancer, hematologic cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, or a soft tissue carcinoma.

60. The method of claim 59, wherein the cancer is osteosarcoma, glioma, astrocytoma, neuroblastoma, cancer of the small intestine, bronchial cancer, small cell lung cancer, non-small cell lung cancer, basal cell carcinoma, or melanoma.

61. The method of claim 59, wherein the cancer is a hematologic cancer.

62. The method of claim 61, wherein the hematologic cancer is acute lymphoblastic leukemia (ALL), acute myeloid leukemia (AML), lymphoma, chronic lymphocytic leukemia (CLL), chronic myelogenous leukemia (CML), Hairy Cell chronic myelogenous leukemia (CML), or multiple myeloma.

63. The method of claim 62, wherein the lymphoma is Hodgkin's lymphoma, non-Hodgkin's lymphoma, Burkitt's lymphoma, or diffuse large B cell lymphoma.

64. The method of any one of claims 58-63, further comprising administering a therapeutically effective amount of a second therapeutic agent.

65. The method of claim 64, wherein the second therapeutic agent is a platinating agent, alkylating agent, anti-cancer antibiotic, antimetabolite, topoisomerase I inhibitor, topoisomerase II inhibitor, or antimicrotubule agent.
